(19) United States
(12) Patent Application Publication
(10) Pub. No.: US 2016/0272617 A1
(43) Pub. Date: Sep. 22, 2016

(54) SUBSTITUTED PHENYLALANINE DERIVATIVES

(71) Applicant: BAYER PHARMA AKTIENGESELLSCHAFT, Berlin (DE)

(72) Inventors: Ulrike RÖHN, Berlin (DE); Manuel ELLERMANN, Berlin (DE); Julia STRASSBURGER, Wuppertal (DE); Astrid WENDT, Syke (DE); Susanne RÖHRIG, Hilden (DE); Robert Alan WEBSTER, Wuppertal (DE); Martina Victoria SCHMIDT, Köln (DE); Adrian TERSTEEGEN, Wuppertal (DE); Kristin BEYER, Kensington, CA (US); Martina SCHÄFER, Berlin (DE); Anja BUCHMÜLLER, Essen (DE); Christoph GERDES, Köln (DE); Michael SPERZEL, Kierspe (DE); Steffen SANDMANN, Essen-Heisingen (DE); Stefan HEITMEIER, Wülfrath (DE); Alexander HILLISCH, Solingen (DE); Jens ACKERSTAFF, Düsseldorf (DE); Carsten TERJUNG, Bochum (DE)

(73) Assignee: Bayer Pharma Aktiengesellschaft, Berlin (DE)

(21) Appl. No.: 15/025,030

(22) PCT Filed: Sep. 24, 2014
(86) PCT No.: PCT/EP2014/070301

§ 371 Date: Mar. 25, 2016

(30) Foreign Application Priority Data
Sep. 26, 2013 (EP) 13186055.3

(51) Int. Cl. C07D 401/12; C07D 257/04; C07D 231/56; C07D 403/12; C07D 451/04; C07D 487/04; C07D 413/12; C07D 263/58; C07D 235/26; C07D 249/04; C07D 235/08; C07D 209/42; C07D 249/08; C07D 401/14

(52) U.S. Cl. CPC C07D 401/12; C07D 249/08; C07D 257/04; C07D 231/56; C07D 403/12; C07D 451/04; C07D 401/14; C07D 413/12; C07D 263/58; C07D 235/26; C07D 249/04; C07D 235/08; C07D 209/42; C07D 487/04

(57) ABSTRACT

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood.

In the more recent past, the traditional theory of two separate regions of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate is low and as a result of the occurrence of TFPI as inhibitor of the TF-FVIIa-FX complex is limited in time.

A central component of the transition from initiation to amplification and propagation of coagulation is factor XIa. In positive feedback loops, thrombin activates, in addition to factor V and factor VIII, also factor XI to factor XIa, whereby factor IX is converted into factor IXa, thus, via the factor IXa/factor VIIIa complex generated in this manner, rapidly producing relatively large amounts of factor Xa. This triggers the production of large amounts of thrombin, leading to strong thrombus growth and stabilizing the thrombus.

The formation of a thrombus or blood clot is counter-regulated by fibrinolysis. Activation of plasminogen by tissue plasminogen activator (tPA) results in formation of the active serine protease, plasmin, which cleaves polymerized fibrin and thus forms the thrombus. This process is referred to as fibrinolysis—with plasmin as key enzyme.

Uncontrolled activation of the coagulation system or defects in the inhibition of the activation processes may cause formation of local thromboses or embolisms in vessels (arteries, veins, lymph vessels) or heart chambers. This may lead to serious thrombotic or thromboembolic disorders. In addition, systemic hypercoagulability may lead to consumption coagulopathy in the context of a disseminated intravasal coagulation.

In the course of many cardiovascular and metabolic disorders, there is an increased tendency for coagulation and platelet activation owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis. This unwanted and excessive haemostasis may, by formation of fibrin- and platelet-rich thrombi, lead to thromboembolic disorders and thrombotic complications with life-threatening conditions.

Thromboembolic disorders are the most frequent cause of morbidity and mortality in most industrialized countries [Heart Disease: A Textbook of Cardiovascular Medicine, Eugene Braunwald, 5. edition, 1997, W.B. Saunders Company, Philadelphia].

The anticoagulants known from the prior art, for example substances for inhibiting or preventing blood coagulation, have various, frequently grave disadvantages. Accordingly, in practice, an efficient treatment method or the prophylaxis of thrombotic/thromboembolic disorders is frequently found to be very difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopenia, alopecia medicomentosa or osteoporosis [Pschyrembel, Klinisches Wörterbuch [clinical dictionary], 257th edition, 1994, Walter de Gruyter Verlag, page 610, keyword “Heparin”; Römpp Lexikon Chemie, version 1.5, 1998, Georg Thieme Verlag Stuttgart, keyword “Heparin” ]. Low-molecular-weight heparins do have a lower probability of leading to the development of heparin-induced thrombocytopenia; however, they can likewise only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other cumarin derivatives which non-selectively inhibit the synthesis of various products of certain vitamin K-dependent coagulation factors in the liver. Owing to the mechanism of action, the onset of action is very slow (latency to the onset of action 36 to 48 hours). The compounds can be administered orally; however, owing to the high risk of bleeding and the narrow therapeutic index complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use, but they have also shown disadvantages, for example highly variable bioavailability, liver damage and bleeding complications.

For antithrombotic medicaments, the therapeutic width is of central importance: The distance between the therapeutically active dose for coagulation inhibition and the dose where bleeding may occur should be as big as possible so that maximum therapeutic activity is achieved at a minimum risk profile.

In various in vivo models with, for example, antibodies as factor XIa inhibitors, but also in factor XIa knock-out models, the antithrombotic effect with small/no prolongation of bleeding time or extension of blood volume was confirmed. In clinical studies, elevated factor XIa concentrations were associated with an increased event rate. However, factor XI deficiency (haemophilia C), in contrast to factor VIIIa or factor IXa (haemophilia A and B, respectively), did not lead to spontaneous bleeding and was only noticed during surgical interventions and traumata. Instead, protection against certain thromboembolic events was found.

In the event of hyperfibrinolytic states, there is inadequate wound closure, which causes severe, sometimes life-threatening, bleeding. This bleeding can be stopped by the inhibition of fibrinolysis with antifibrinolytics, by which plasmin activity is reduced. Corresponding effects with the plasminogen inhibitor tranexamic acid have been shown in various clinical studies.

It is therefore an object of the present invention to provide novel compounds for treatment and/or prophylaxis of cardiovascular disorders and/or severe perioperative blood loss in man and animals, said compounds having a wide therapeutic range.

WO89/11852 describes, inter alia, substituted phenylalanine derivatives for treatment of pancreatitis, and WO 2007/070816 describes substituted thiophene derivatives as factor XIa inhibitors.

The invention provides compounds of the formula

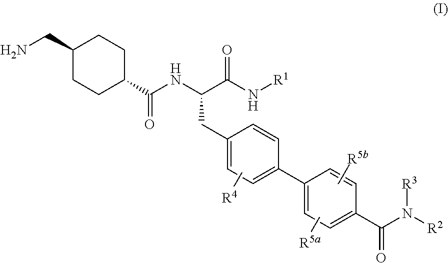

in which

R¹ is a group of the formula

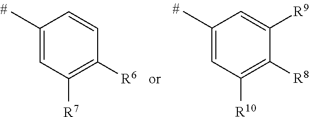

where # is the attachment site to the nitrogen atom,
R⁶ is 5-membered heteroaryl,
   where heteroaryl may be substituted by a substituent selected from the group consisting of oxo, chlorine, cyano, hydroxyl and C₁-C₃-alkyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl, methoxy, C₁-C₄-alkoxycarbonyl, aminocarbonyl and C₁-C₃-alkylaminocarbonyl, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
   where the heterocycle may be substituted by 1 to 2 substituents selected independently from the group consisting of oxo, chlorine, cyano, hydroxyl, hydroxycarbonyl, C₁-C₃-alkyl, pyrazolyl and pyridyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent from the group consisting of hydroxyl, amino, hydroxycarbonyl, methoxy, C₁-C₄-alkoxycarbonyl, aminocarbonyl and C₁-C₃-alkylaminocarbonyl, and in which alkyl is additionally substituted by 1 to 6 fluorine substituents,
R¹⁰ is hydrogen, fluorine, chlorine or hydroxycarbonyl,

R² is hydrogen, C₁-C₆-alkyl, C₃-C₆-cycloalkyl, or 4- to 8-membered heterocyclyl bonded via a carbon atom,
where alkyl may be substituted by 1 to 2 substituents selected independently from the group consisting of fluorine, hydroxyl, amino, hydroxycarbonyl, C₁-C₃-alkylamino, difluoromethyl, trifluoromethyl, —(OCH₂CH₂)ₙ—OCH₃, —(OCH₂CH₂)ₘ—OH, trimethylaminium, pyrrolidinyl, C₃-C₆-cycloalkyl, 4- to 8-membered heterocyclyl bonded via a carbon atom, and 4- to 6-membered heterocyclylcarbonyl,
in which n is a number from 1 to 6,
in which m is a number from 1 to 6,
   in which heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
and
   in which heterocyclylcarbonyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
and
where cycloalkyl may be substituted by 1 to 3 substituents selected independently from the group consisting of oxo, fluorine, hydroxyl, amino, C₁-C₄-alkyl and C₁-C₃-alkylamino,
   in which alkyl and alkylamino may be substituted by 1 to 5 fluorine substituents or one phenyl substituent,
and
where heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
   where alkyl and alkylamino may be substituted by 1 to 5 substituents selected independently from the group consisting of hydroxyl and fluorine,
and where heterocyclyl may additionally be substituted by 1 to 4 substituents independently selected from the group consisting of fluorine and methyl,
R³ is hydrogen or C₁-C₃-alkyl,
or
R² and R³ together with the nitrogen atom to which they are bonded form a 4- to 8-membered heterocycle,
   where the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,

R⁴ is hydrogen, fluorine, chlorine, methyl or methoxy,

R^{5a} is hydrogen, fluorine, chlorine, C₁-C₄-alkyl, methoxy, ethoxy or trifluoromethyl,

R^{5b} is hydrogen, fluorine, methyl or methoxy,

and the salts thereof, the solvates thereof and the solvates of the salts thereof.

Inventive compounds are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The inventive compounds may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner, chromatography processes are preferably used for this, especially HPLC chromatography on an achiral or chiral phase.

If the inventive compounds can occur in tautomeric forms, the present invention encompasses all the tautomeric forms.

The present invention also encompasses all suitable isotopic variants of the inventive compounds. An isotopic variant of an inventive compound is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulphur, fluorine, chlorine, bromine and iodine, such as ²H (deuterium), ³H (tritium), ³C, ¹⁴C, ¹⁵N, ¹⁷O, ¹⁸O, ³², ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the inventive compounds can be prepared by the processes known to those skilled in the art, for example by the methods described below and the procedures described in the working examples, by using corresponding isotopic modifications of the respective reagents and/or starting compounds.

In the context of the present invention, preferred salts are physiologically acceptable salts of the inventive compounds. The invention also encompasses salts which themselves are unsuitable for pharmaceutical applications but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

In the context of the invention, solvates refer to those forms of the inventive compounds which, in the solid or liquid state, form a complex by coordination with solvent molecules. Hydrates are a specific form of the solvates in which the coordination is with water.

In addition, the present invention also encompasses prodrugs of the inventive compounds. The term “prodrugs” includes compounds which may themselves be biologically active or inactive but are converted t inventive compounds while resident in the body (for example metabolically or hydrolytically).

The two ways (A) and (B) of representing a 1,4-disubstituted cyclohexyl derivative shown below are equivalent to one another and identical, and in both cases describe a trans-1,4-disubstituted cyclohexyl derivative.

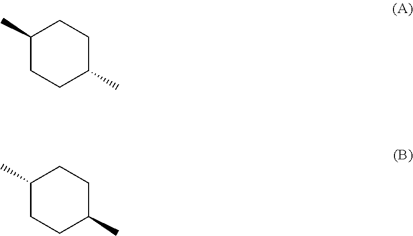

This applies especially to the structural element of tranexamamide, for example N-[(trans-4-{[(tert-butoxycarbonyl)amino]methyl}cyclohexyl)carbonyl and trans-4-(aminomethyl)-cyclohexyl]carbonyl}. This also applies to the structural element of trans-4-hydroxycyclohexylamine, for example in (trans-4-hydroxycyclohexyl)carbamoyl. In the present invention, representation (A) is used with preference for tranexamamide.

The three ways (C), (D) and (E) of representing tautomers of a triazole derivative shown below are equivalent to one another and identical and in all cases describe a 1,4-disubstituted triazole derivative.

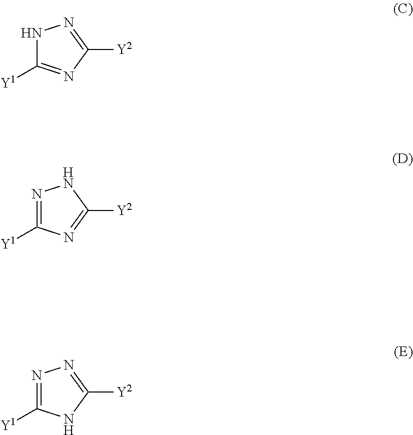

This applies especially to the following structural elements: 1H-1,2,4-triazol-3-yl, 1H-1,2,4-triazol-5-yl, 4H-1,2,4-triazol-3-yl and 4H-1,2,4-triazol-5-yl. Y¹ and Y² here are different substituents.

The two ways (F) and (G) of representing tautomers of a tetrazole derivative shown below are equivalent to one another and identical and in all cases describe a tetrazole derivative.

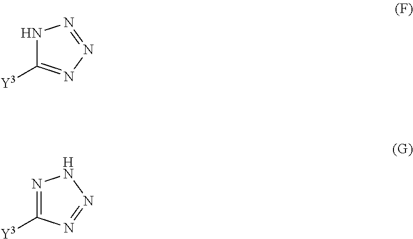

This applies especially to the following structural elements: 1H-tetrazol-5-yl and 2H-tetrazol-5-yl. Y³ here is the remainder of the compound.

The inventive compounds of the formula

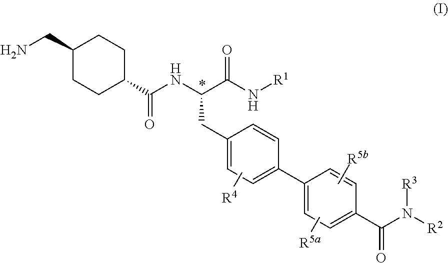

and all L-phenylalanine intermediates are described as the (S) configuration at the stereocentre marked with an * in the above formula, since L-phenylalanine derivatives are introduced into the synthesis as central units. In the preparation of the inventive compounds, the coupling of the L-phenylalanine intermediates with the amine H₂N—R¹ can result in partial epimerization at the stereocentre marked by an *. Thus, a mixture of the inventive compounds of (S) enantiomer and (R) enantiomer can arise. The main component is the (S) enantiomer depicted in each case. The mixtures of (S) enantiomer and (R) enantiomer can be separated into their enantiomers by methods known to those skilled in the art, for example by chromatography on a chiral phase.

The enantiomers can be separated either directly after the coupling of the L-phenylalanine intermediates with the amine H₂N—R¹ or at a later synthesis intermediate, or else the inventive compounds can be separated themselves. Preference is given to the separation of the enantiomers directly after the coupling of the L-phenylalanine intermediates with the amine H₂N—R¹.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, the substituents, unless specified otherwise, each have the following meaning:

Alkyl is a linear or branched alkyl radical having 1 to 6 carbon atoms, preferably 1 to 4 carbon atoms, more preferably 1 to 3 carbon atoms, by way of example and with preference methyl, ethyl, n-propyl, isopropyl, 2-methylprop-1-yl, n-butyl, tert-butyl, n-pentyl and n-hexyl.

Alkoxy is a linear or branched alkoxy radical having 1 to 6 carbon atoms, preferably 1 to 4 carbon atoms, more preferably 1 to 3 carbon atoms, by way of example and with preference methoxy, ethoxy, n-propoxy, isopropoxy, 2-methylprop-1-oxy, n-butoxy, tert-butoxy, n-pentoxy and n-hexoxy.

Alkylamino is an amino group having one or two independently selected, identical or different, linear or branched alkyl radicals each having 1 to 3 carbon atoms, for example and with preference methylamino, ethylamino, n-propylamino, isopropylamino, N,N-dimethylamino, N,N-diethylamino, N-ethyl-N-methylamino, N-methyl-N-n-propylamino, N-isopropyl-N-n-propylamino and N,N-diisopropylamino. C₁-C₃-Alkylamino is, for example, a monoalkylamino radical having 1 to 3 carbon atoms or a dialkylamino radical having 1 to 3 carbon atoms in each alkyl radical.

Alkoxycarbonyl is a linear or branched alkoxy radical bonded by a carbonyl group, having 1 to 4 carbon atoms, preferably 1 to 3 carbon atoms, for example and with preference methoxycarbonyl, ethoxycarbonyl, n-propoxycarbonyl, isopropoxycarbonyl, n-butoxycarbonyl and tert-butoxycarbonyl.

Alkylaminocarbonyl is an amino group having one or two independently selected, identical or different, straight-chain or branched alkyl substituents each having 1 to 3 carbon atoms, bonded via a carbonyl group, for example and with preference methylaminocarbonyl, ethylaminocarbonyl, n-propylaminocarbonyl, isopropylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N,N-diisopropylaminocarbonyl. C₁-C₃-Alkylaminocarbonyl is, for example, a monoalkylaminocarbonyl radical having 1 to 3 carbon atoms or a dialkylaminocarbonyl radical having 1 to 3 carbon atoms in each alkyl substituent.

Cycloalkyl is a monocyclic cycloalkyl group having 3 to 6 carbon atoms, preferred examples of cycloalkyl being cyclopropyl, cyclobutyl, cyclopentyl and cyclohexyl.

4- to 8-membered heterocyclyl bonded via a carbon atom in the definition of the R² radical is a saturated or partly unsaturated, monocyclic or bicyclic radical bonded via a carbon atom, having 4 to 8 ring atoms, preferably 5 or 6 ring atoms, and up to 3 heteroatoms and/or hetero groups, preferably 1 or 2 heteroatoms and/or hetero groups, from the group of S, O, N, SO and SO₂, where one nitrogen atom may also form an N-oxide, for example and with preference azetidinyl, pyrrolidinyl, piperidinyl, tetrahydropyranyl, 3-azabicyclo[3.1.0]hex-6-yl, 8-azabicyclo[3.2.1]oct-3-yl and azepanyl, more preferably pyrrolidinyl, piperidinyl, 3-azabicyclo[3.1.0]hex-6-yl, 8-azabicyclo[3.2.1]oct-3-yl and octahydrocyclopenta[b]pyrrol-4-yl.

4- to 6-membered heterocyclylcarbonyl in the definition of the R² radical is a saturated or partly unsaturated, monocyclic radical bonded via a carbonyl group, having 4 to 6 ring atoms, preferably 5 or 6 ring atoms, and up to 3 heteroatoms and/or hetero groups, preferably 1 or 2 heteroatoms and/or hetero groups, from the group of S, O, N, SO and SO₂, where one nitrogen atom may also form an N-oxide, for example and with preference azetidinyl, pyrrolidinyl, piperidinyl and piperazinyl, more preferably pyrrolidinyl and piperidinyl.

4- to 8-membered heterocycle in the definition of the R² and R³ radicals is a saturated or partly unsaturated, monocyclic or bicyclic radical having 4 to 8 ring atoms, preferably 4 to 7 ring atoms, more preferably 5 or 6 ring atoms, and up to 3 heteroatoms and/or hetero groups, preferably 1 or 2 heteroatoms and/or hetero groups, from the group of S, O, N, SO and SO₂, where one nitrogen atom may also form an N-oxide, for example and with preference azetidinyl, pyrrolidinyl, morpholinyl, thiomorpholinyl, piperidinyl, piperazinyl, 3-azabicyclo[3.1.0]hex-6-yl, 8-azabicyclo[3.2.1]oct-3-yl, azepanyl and hexahydropyrrolo[3,4-b]pyrrol-1(2H)-yl, more preferably pyrrolidinyl and piperazinyl.

5-membered heteroaryl in the definition of the R⁶ radical is an aromatic monocyclic radical having 5 ring atoms and up to 4 heteroatoms and/or hetero groups from the group of S, ON, SO and SO₂, where one nitrogen atom may also form an N-oxide, for example and with preference thienyl, furyl, pyrrolyl, thiazolyl, oxazolyl, isoxazolyl, oxadiazolyl, pyrazolyl, imidazolyl, triazolyl and tetrazolyl, more preferably imidazolyl, triazolyl and tetrazolyl.

5-membered heterocycle in the definition of the R⁸ and R⁹ radicals is a saturated, partly unsaturated or aromatic monocyclic radical having 5 ring atoms and up to 2 heteroatoms and/or hetero groups from the group of S, O, N, SO and SO₂, where one nitrogen atom may also form an N-oxide. This 5-membered heterocycle together with the phenyl ring to which it is bonded is, for example and with preference, 2,3-dihydro-1-benzothiophen-5-yl, 1,3-dihydro-2-benzothiophen-5-yl, 2,3-dihydro-1-benzofuran-5-yl, 1,3-dihydro-2-benzofuran-5-yl, indolin-5-yl, isoindolin-5-yl, 2,3-dihydro-1H-indazol-5-yl, 2,3-dihydro-1H-benzimidazol-5-yl, 1,3-dihydro-2,1-benzoxazol-5-yl, 2,3-dihydro-1,3-benzoxazol-5-yl, 1,3-dihydro-2,1-benzothiazol-5-yl, 2,3-dihydro-1,3-benzothiazol-5-yl, 1H-benzimidazol-5-yl, 1H-indazol-5-yl, 1,2-benzoxazol-5-yl, indol-5-yl, isoindol-5-yl, benzofuran-5-yl, benzothiophen-5-yl, 1H-benzotriazol-5-yl, 2,3-dihydro-1-benzothiophen-6-yl, 1,3-dihydro-2-benzothiophen-6-yl, 2,3-dihydro-1-benzofuran-6-yl, 1,3-dihydro-2-benzofuran-6-yl, indolin-6-yl, isoindolin-6-yl, 2,3-dihydro-1H-indazol-6-yl, 2,3-dihydro-1H-benzimidazol-6-yl, 1,3-dihydro-2,1-benzoxazol-6-yl, 2,3-dihydro-1,3-benzoxazol-6-yl, 1,3-dihydro-2,1-benzothiazol-6-yl, 2,3-dihydro-1,3-benzothiazol-6-yl, 1H-benzimidazol-6-yl, 1H-indazol-6-yl, 1,2-benzoxazol-6-yl, indol-6-yl, isoindol-6-yl, benzofuran-6-yl, benzothiophen-6-yl and 1H-benzotriazol-6-yl, more preferably 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl.

In the formulae of the group which may represent R¹, the end point of the line marked by # in each case does not represent a carbon atom or a CH₂ group, but is part of the bond to the atom to which R¹ is bonded.

Preference is given to compounds of the formula (I) in which

R¹ is a group of the formula

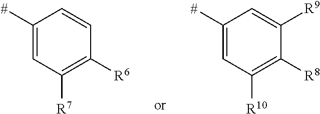

where # is the attachment site to the nitrogen atom,
R⁶ is 5-membered heteroaryl,
   where heteroaryl may be substituted by a substituent selected from the group consisting of chlorine and C₁-C₃-alkyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl, methoxy, C₁-C₄-alkoxycarbonyl, aminocarbonyl and C₁-C₃-alkylaminocarbonyl, and in which alkyl is additionally substituted by 1 to 6 fluorine substituents,
R⁷ is hydrogen or fluorine,
R⁸ and R⁹ together with the carbon atoms to which they are bonded form a 5-membered heterocycle,
   where the heterocycle may be substituted by 1 to 2 substituents selected independently from the group consisting of oxo, chlorine, hydroxyl, hydroxycarbonyl, C₁-C₃-alkyl, pyrazolyl and pyridyl,
      in which alkyl may be substituted by 1 to 3 substituents selected independently from the group consisting of hydroxycarbonyl and methoxy,
      or
      in which alkyl may be substituted by 1 to 7 fluorine substituents,
      or
      in which alkyl is substituted by a substituent from the group consisting of hydroxyl, amino, hydroxycarbonyl, methoxy, C₁-C₄-alkoxycarbonyl, aminocarbonyl and C₁-C₃-alkylaminocarbonyl, and in which alkyl is additionally substituted by 1 to 6 fluorine substituents,
R¹⁰ is hydrogen, fluorine, chlorine or hydroxycarbonyl,
R² is hydrogen, C₁-C₆-alkyl, C₃-C₆-cycloalkyl, or 4- to 8-membered heterocyclyl bonded via a carbon atom,
   where alkyl may be substituted by 1 to 2 substituents selected independently from the group consisting of fluorine, hydroxyl, amino, C₁-C₃-alkylamino, difluoromethyl, trifluoromethyl, —(OCH₂CH₂)ₙ—OCH₃, trimethylaminium and pyrrolidinyl,
   in which n is a number from 1 to 6,
   and
   where cycloalkyl may be substituted by 1 to 3 substituents selected independently from the group consisting of oxo, fluorine, hydroxyl, amino, C₁-C₄-alkyl and C₁-C₃-alkylamino,
      in which alkyl and alkylamino may be substituted by 1 to 5 fluorine substituents,
   and
   where heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
      where alkyl and alkylamino may be substituted by 1 to 5 substituents selected independently from the group consisting of hydroxyl and fluorine,
   and where heterocyclyl may additionally be substituted by 1 to 4 substituents independently selected from the group consisting of fluorine and methyl,
R³ is hydrogen or C₁-C₃-alkyl,
or
R² and R³ together with the nitrogen atom to which they are bonded form a 4- to 8-membered heterocycle,
   where the heterocycle may be substituted by 1 to 2 substituents selected independently from the group consisting of oxo, fluorine, hydroxyl, C₁-C₄-alkyl and C₁-C₃-alkylamino,
R⁴ is hydrogen, fluorine, chlorine, methyl or methoxy,
R^{5a} is hydrogen, fluorine, chlorine, C₁-C₄-alkyl, methoxy, ethoxy or trifluoromethyl,
R^{5b} is hydrogen, fluorine, methyl or methoxy,
and the salts thereof, the solvates thereof and the solvates of the salts thereof.

Preference is also given to compounds of the formula (I) in which

R¹ is a group of the formula

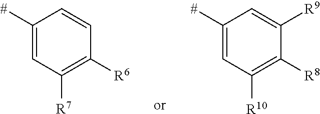

where # is the attachment site to the nitrogen atom,
R⁶ is 5-membered heteroaryl,
   where heteroaryl may be substituted by a substituent selected from the group consisting of C₁-C₃-alkyl,
      in which alkyl may be substituted by a hydroxycarbonyl substituent,
      or
      in which alkyl is substituted by a hydroxycarbonyl substituent and in which alkyl is additionally substituted by 1 to 6 fluorine substituents,
R⁷ is hydrogen,
R⁸ and R⁹ together with the carbon atoms to which they are bonded form a 5-membered heterocycle,
   where the heterocycle may be substituted by 1 to 2 substituents selected independently from the group consisting of oxo, hydroxyl, methyl, ethyl and n-propyl,
      in which methyl, ethyl and n-propyl may be substituted by a hydroxycarbonyl substituent,
      or
      in which ethyl and n-propyl may be substituted by 4 to 7 fluorine substituents,
      or
      in which ethyl and n-propyl are substituted by a hydroxycarbonyl substituent and in which ethyl and n-propyl are additionally substituted by 4 to 6 fluorine substituents,
R¹⁰ is hydrogen, fluorine or chlorine,
R² is hydrogen, C₁-C₆-alkyl, C₃-C₆-cycloalkyl, or 4- to 8-membered heterocyclyl bonded via a carbon atom,
   where alkyl may be substituted by 1 to 2 substituents selected independently from the group consisting of amino, C₁-C₃-alkylamino and trifluoromethyl,
   and
   where cycloalkyl may be substituted by 1 to 2 substituents selected independently from the group consisting of hydroxyl, amino, methyl and C₁-C₃-alkylamino,
   and
   where heterocyclyl may be substituted by 1 to 2 substituents selected independently from the group consisting of oxo, fluorine, hydroxycarbonyl, C₁-C₄-alkyl, C₁-C₃-alkylamino, 2,2,2-trifluoroeth-1-yl and C₁-C₄-alkoxycarbonyl,
      in which alkyl may be substituted by a hydroxyl substituent,
   and where heterocyclyl may additionally be substituted by 1 to 2 substituents independently selected from the group consisting of fluorine and methyl,
R³ is hydrogen,
or
R² and R³ together with the nitrogen atom to which they are bonded form a 4- to 6-membered heterocycle,
R⁴ is hydrogen or fluorine,
R^{5a} is hydrogen, fluorine, chlorine, methyl, methoxy or trifluoromethyl,
R^{5b} is hydrogen, fluorine, methyl or methoxy, and the salts thereof, the solvates thereof and the solvates of the salts thereof.

Preference is also given to compounds of the formula (I) in which

in which

R¹ is a group of the formula

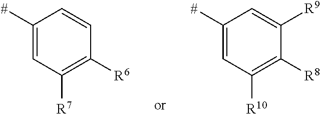

where # is the attachment site to the nitrogen atom,
R⁶ is 5-membered heteroaryl,
   where heteroaryl may be substituted by a substituent selected from the group consisting of oxo, chlorine, cyano, hydroxyl and C₁-C₃-alkyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
   where the heterocycle may be substituted by 1 to 2 substituents selected independently from the group consisting of oxo, chlorine, cyano, hydroxyl, C₁-C₃-alkyl, pyrazolyl and pyridyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R¹⁰ is hydrogen, fluorine or chlorine,
R² is hydrogen, C₁-C₆-alkyl, C₃-C₆-cycloalkyl, or 4- to 8-membered heterocyclyl bonded via a carbon atom,
   where alkyl may be substituted by 1 to 2 substituents selected from the group consisting of fluorine, hydroxyl, amino, hydroxycarbonyl, C₁-C₃-alkylamino, difluoromethyl, trifluoromethyl, —(OCH₂CH₂)ₙ—OCH₃, —(OCH₂CH₂)ₘ—OH, trimethylaminium and pyrrolidinyl,
   in which n is a number from 1 to 6,
   in which m is a number from 1 to 6,
   and
   where cycloalkyl may be substituted by 1 to 2 substituents selected from the group consisting of oxo, fluorine, hydroxyl, amino, C₁-C₄-alkyl and C₁-C₃-alkylamino,
      in which alkyl and alkylamino may be substituted by 1 to 5 fluorine substituents, and
   where heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
      where alkyl and alkylamino may be substituted by 1 to 5 substituents selected independently from the group consisting of hydroxyl and fluorine,
   and where heterocyclyl may additionally be substituted by 1 to 4 substituents independently selected from the group consisting of fluorine and methyl,
R³ is hydrogen or C₁-C₃-alkyl,
or
R² and R³ together with the nitrogen atom to which they are bonded form a 4- to 7-membered heterocycle,
   where the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R⁴ is hydrogen, fluorine, chlorine, methyl or methoxy,
R^{5a} is hydrogen, fluorine, chlorine, C₁-C₄-alkyl, methoxy or trifluoromethyl,
R^{5b} is hydrogen,
and the salts thereof, the solvates thereof and the solvates of the salts thereof.

Preference is also given to compounds of the formula (I) in which

R¹ is a group of the formula

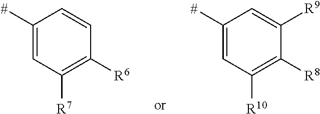

where # is the attachment site to the nitrogen atom,
R⁶ is 5-membered heteroaryl,
   where heteroaryl may be substituted by a substituent selected from the group consisting of chlorine and C₁-C₃-alkyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁷ is hydrogen or fluorine,
R⁸ and R⁹ together with the carbon atoms to which they are bonded form a 5-membered heterocycle,
   where the heterocycle may be substituted by 1 to 2 substituents selected independently from the group consisting of oxo, chlorine, hydroxyl, C₁-C₃-alkyl, pyrazolyl and pyridyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R¹⁰ is hydrogen or fluorine,
R² is hydrogen, C₁-C₆-alkyl, C₃-C₆-cycloalkyl, or 4- to 8-membered heterocyclyl bonded via a carbon atom,
   where alkyl may be substituted by 1 to 2 substituents selected independently from the group consisting of fluorine, hydroxyl, amino, C₁-C₃-alkylamino, difluoromethyl, trifluoromethyl, —(OCH₂CH₂)ₙ—OCH₃, trimethylaminium and pyrrolidinyl,
   in which n is a number from 1 to 6,
   and
   where cycloalkyl may be substituted by 1 to 2 substituents selected independently from the group consisting of oxo, fluorine, hydroxyl, amino, C₁-C₄-alkyl and C₁-C₃-alkylamino,
      in which alkyl and alkylamino may be substituted by 1 to 5 fluorine substituents, and
   where heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
      where alkyl and alkylamino may be substituted by 1 to 5 substituents selected independently from the group consisting of hydroxyl and fluorine,
   and where heterocyclyl may additionally be substituted by 1 to 4 substituents independently selected from the group consisting of fluorine and methyl,
R³ is hydrogen or C₁-C₃-alkyl,
or
R² and R³ together with the nitrogen atom to which they are bonded form a 4- to 7-membered heterocycle,
   where the heterocycle may be substituted by 1 to 2 substituents selected independently from the group consisting of oxo, fluorine, hydroxyl, C₁-C₄-alkyl and C₁-C₃-alkylamino,
R⁴ is hydrogen, fluorine, chlorine, methyl or methoxy,
R^{5a} is hydrogen, fluorine, chlorine, methyl, ethyl, methoxy or trifluoromethyl,
R^{5b} is hydrogen,
and the salts thereof, the solvates thereof and the solvates of the salts thereof.

Preference is also given to compounds of the formula (I) in which

R¹ is a group of the formula

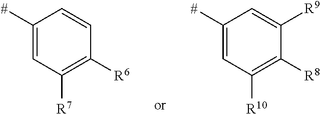

where # is the attachment site to the nitrogen atom,
R⁶ is 5-membered heteroaryl,
   where heteroaryl may be substituted by a substituent selected from the group consisting of C₁-C₃-alkyl,
      in which alkyl may be substituted by a hydroxycarbonyl substituent,
      or
      in which alkyl is substituted by a hydroxycarbonyl substituent and in which alkyl is additionally substituted by 1 to 6 fluorine substituents,
R⁷ is hydrogen,
R⁸ and R⁹ together with the carbon atoms to which they are bonded form a 5-membered heterocycle,
   where the heterocycle may be substituted by 1 to 2 substituents selected independently from the group consisting of oxo, hydroxyl, methyl, ethyl and n-propyl,
      in which methyl, ethyl and n-propyl may be substituted by a hydroxycarbonyl substituent,
      or
      in which ethyl and n-propyl may be substituted by 4 to 7 fluorine substituents,
      or
      in which ethyl and n-propyl are substituted by a hydroxycarbonyl substituent and in which ethyl and n-propyl are additionally substituted by 4 to 6 fluorine substituents,
R¹⁰ is hydrogen or fluorine,
R² is hydrogen, C₁-C₆-alkyl, C₃-C₆-cycloalkyl, or 4- to 8-membered heterocyclyl bonded via a carbon atom,
   where alkyl may be substituted by 1 to 2 substituents selected independently from the group consisting of amino and C₁-C₃-alkylamino,
   and
   where cycloalkyl may be substituted by 1 to 2 substituents selected independently from the group consisting of amino, methyl and C₁-C₃-alkylamino,
   and
   where heterocyclyl may be substituted by 1 to 2 substituents selected independently from the group consisting of oxo, fluorine, hydroxycarbonyl, C₁-C₄-alkyl, C₁-C₃-alkylamino, 2,2,2-trifluoroeth-1-yl and C₁-C₄-alkoxycarbonyl,
      in which alkyl may be substituted by a hydroxyl substituent,
   and where heterocyclyl may additionally be substituted by 1 to 2 substituents independently selected from the group consisting of fluorine and methyl,
R³ is hydrogen,
or
R² and R³ together with the nitrogen atom to which they are bonded form a 5- or 6-membered heterocycle,
R⁴ is hydrogen or fluorine,
R^{5a} is hydrogen, chlorine, methyl, methoxy or trifluoromethyl,
R^{5b} is hydrogen,
and the salts thereof, the solvates thereof and the solvates of the salts thereof.

Preference is also given to compounds of the formula (I) in which

R¹ is a group of the formula

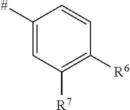

where # is the attachment site to the nitrogen atom,
R⁶ is triazolyl or tetrazolyl,
   where triazolyl may be substituted by a substituent selected from the group consisting of methyl, ethyl and n-propyl,
      in which methyl, ethyl and n-propyl may be substituted by a hydroxycarbonyl substituent,
      or
      in which ethyl and n-propyl are substituted by a hydroxycarbonyl substituent and in which ethyl and n-propyl are additionally substituted by 4 to 6 fluorine substituents,
R⁷ is hydrogen,
or
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl, 1H-indazol-6-yl, 7-fluoro-2,3-dihydro-1H-benzimidazol-5-yl, 7-fluoro-2,3-dihydro-1,3-benzoxazol-5-yl or 4-fluoro-2,3-dihydro-1H-indazol-6-yl,
   where 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl, 1H-indazol-6-yl, 7-fluoro-2,3-dihydro-1H-benzimidazol-5-yl, 7-fluoro-2,3-dihydro-1,3-benzoxazol-5-yl and 4-fluoro-2,3-dihydro-1H-indazol-6-yl may be substituted by 1 to 2 substituents selected independently from the group consisting of oxo, methyl, ethyl and n-propyl,
      in which ethyl and n-propyl may be substituted by 4 to 7 fluorine substituents,
      or
      in which ethyl and n-propyl are substituted by a hydroxycarbonyl substituent and in which ethyl and n-propyl are additionally substituted by 4 to 6 fluorine substituents,
R² is hydrogen, C₁-C₄-alkyl, cyclopropyl, cyclohexyl, or heterocyclyl bonded via a carbon atom, selected from the group of pyrrolidinyl, piperidinyl, 3-azabicyclo[3.1.0]hex-6-yl and 8-azabicyclo[3.2.1]oct-3-yl,
   where alkyl may be substituted by 1 to 2 substituents selected independently from the group consisting of amino and C₁-C₃-alkylamino, and
   where cyclohexyl may be substituted by a substituent selected from the group consisting of amino and C₁-C₃-alkylamino,
   and
   where pyrrolidinyl, piperidinyl, 3-azabicyclo[3.1.0]hex-6-yl and 8-azabicyclo[3.2.1]oct-3-yl may be substituted by 1 to 2 substituents independently selected from the group consisting of oxo, fluorine, C₁-C₄-alkyl and 2,2,2-trifluoroeth-1-yl,
R³ is hydrogen,
or
R² and R³ together with the nitrogen atom to which they are bonded form a pyrrolidinyl or piperazinyl,
R⁴ is hydrogen or fluorine,
R^{5a} is hydrogen, chlorine, methyl, methoxy or trifluoromethyl,
R^{5b} is hydrogen,
and the salts thereof, the solvates thereof and the solvates of the salts thereof.

Preference is also given to compounds of the formula (I) in which

R¹ is a group of the formula

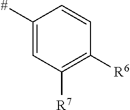

where # is the attachment site to the nitrogen atom,
R⁶ is triazolyl or tetrazolyl,
   where triazolyl may be substituted by a substituent selected from the group consisting of methyl, ethyl and n-propyl,
      in which methyl, ethyl and n-propyl may be substituted by a hydroxycarbonyl substituent,
      or
      in which ethyl and n-propyl are substituted by a hydroxycarbonyl substituent and in which ethyl and n-propyl are additionally substituted by 4 to 6 fluorine substituents,
R⁷ is hydrogen,
or
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl or 1H-indazol-6-yl,
   where the 5-membered heterocycle in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by 1 to 2 substituents selected independently from the group consisting of oxo, methyl, ethyl and n-propyl,
      in which ethyl and n-propyl may be substituted by 4 to 7 fluorine substituents,
      or
      in which ethyl and n-propyl are substituted by a hydroxycarbonyl substituent and in which ethyl and n-propyl are additionally substituted by 4 to 6 fluorine substituents,
   and
   where the benzyl ring in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by a substituent selected from the group consisting of fluorine, chlorine and hydroxycarbonyl,
R² is hydrogen, C₁-C₄-alkyl, cyclopropyl, cyclohexyl, or heterocyclyl bonded via a carbon atom, selected from the group of pyrrolidinyl, piperidinyl, 3-azabicyclo[3.1.0]hex-6-yl and 8-azabicyclo[3.2.1]oct-3-yl,
   where alkyl may be substituted by 1 to 2 substituents selected independently from the group consisting of amino, C₁-C₃-alkylamino and trifluoromethyl,
   and
   where cyclohexyl may be substituted by a substituent selected from the group consisting of amino and C₁-C₃-alkylamino,
   and
   where pyrrolidinyl, piperidinyl, 3-azabicyclo[3.1.0]hex-6-yl and 8-azabicyclo[3.2.1]oct-3-yl may be substituted by 1 to 2 substituents independently selected from the group consisting of oxo, fluorine, C₁-C₄-alkyl and 2,2,2-trifluoroeth-1-yl,
R³ is hydrogen,
or
R² and R³ together with the nitrogen atom to which they are bonded form a pyrrolidinyl or piperazinyl,
R⁴ is hydrogen or fluorine,
R^{5a} is hydrogen, chlorine, methyl, methoxy or trifluoromethyl,
R^{5b} is hydrogen,
and the salts thereof, the solvates thereof and the solvates of the salts thereof.

Preference is also given to compounds of the formula (I) in which

R¹ is a group of the formula

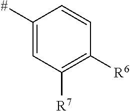

where # is the attachment site to the nitrogen atom,
R⁶ is triazolyl,
   where triazolyl is substituted by a substituent selected from the group consisting of ethyl and n-propyl,
      in which ethyl and n-propyl are substituted by a hydroxycarbonyl substituent and in which ethyl and n-propyl are additionally substituted by 4 to 6 fluorine substituents,
R⁷ is hydrogen,
R² is ethyl, isopropyl, cyclopropyl, cyclobutyl, cyclohexyl, or heterocyclyl bonded via a carbon atom, selected from the group of pyrrolidinyl and piperidinyl,
   where ethyl is substituted by a trifluoromethyl substituent,
   and
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo, fluorine, C₁-C₄-alkyl and 2,2,2-trifluoroeth-1-yl,
R³ is hydrogen,
or
R² and R³ together with the nitrogen atom to which they are bonded form a pyrrolidinyl or piperazinyl,
R⁴ is hydrogen or fluorine,
R^{5a} is hydrogen, fluorine, chlorine, methyl, methoxy or trifluoromethyl,
R^{5b} is hydrogen,
and the salts thereof, the solvates thereof and the solvates of the salts thereof.

Preference is also given to compounds of the formula (I) in which

R¹ is a group of the formula

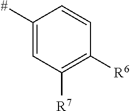

where # is the attachment site to the nitrogen atom,
R⁶ is triazolyl,
   where triazolyl is substituted by a substituent selected from the group consisting of ethyl and n-propyl,
      in which ethyl and n-propyl are substituted by a hydroxycarbonyl substituent and in which ethyl and n-propyl are additionally substituted by 4 to 6 fluorine substituents,
R⁷ is hydrogen,
R² is ethyl, isopropyl, cyclopropyl, cyclobutyl, cyclohexyl, or heterocyclyl bonded via a carbon atom, selected from the group of pyrrolidinyl and piperidinyl,
   where ethyl is substituted by a trifluoromethyl substituent,
   and
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo and methyl,
R³ is hydrogen,
R⁴ is hydrogen or fluorine,
R^{5a} is fluorine or methyl,
R^{5b} is hydrogen,
and the salts thereof, the solvates thereof and the solvates of the salts thereof.

Preference is also given to compounds of the formula (I) in which

R¹ is a group of the formula

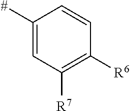

where # is the attachment site to the nitrogen atom,
R⁶ is triazolyl,
   where triazolyl is substituted by a substituent selected from the group consisting of ethyl and n-propyl,
      in which ethyl and n-propyl are substituted by a hydroxycarbonyl substituent and in which ethyl and n-propyl are additionally substituted by 4 to 6 fluorine substituents,
R⁷ is hydrogen,
R² is cyclohexyl, or heterocyclyl bonded via a carbon atom, selected from the group of pyrrolidinyl and piperidinyl,
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo and methyl,
R³ is hydrogen,
R⁴ is hydrogen or fluorine,
R^{5a} is fluorine or methyl,
R^{5b} is hydrogen,
and the salts thereof, the solvates thereof and the solvates of the salts thereof.

Preference is also given to compounds of the formula (I) in which

R¹ is a group of the formula

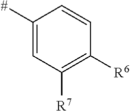

where # is the attachment site to the nitrogen atom,
R⁶ is triazolyl,
   where triazolyl is substituted by a substituent selected from the group consisting of ethyl and n-propyl,
      in which ethyl and n-propyl are substituted by a hydroxycarbonyl substituent and in which ethyl and n-propyl are additionally substituted by 4 to 6 fluorine substituents,
R⁷ is hydrogen,
R² is cyclohexyl,
   where cyclohexyl is substituted by a substituent selected from the group consisting of hydroxyl, amino and C₁-C₃-alkylamino,
R³ is hydrogen,
R⁴ is hydrogen or fluorine,
R^{5a} is fluorine or methyl,
R^{5b} is hydrogen,
and the salts thereof, the solvates thereof and the solvates of the salts thereof.

Preference is also given to compounds of the formula (I) in which

R¹ is a group of the formula

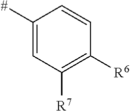

where # is the attachment site to the nitrogen atom,
R⁶ is triazolyl,
   where triazolyl is substituted by a substituent selected from the group consisting of ethyl and n-propyl,
      in which ethyl and n-propyl are substituted by a hydroxycarbonyl substituent and in which ethyl and n-propyl are additionally substituted by 4 to 6 fluorine substituents,
R⁷ is hydrogen,
R² is heterocyclyl bonded via a carbon atom, selected from the group of pyrrolidinyl and piperidinyl,
   where pyrrolidinyl and piperidinyl may be substituted by 1 to 2 substituents independently selected from the group consisting of oxo and methyl,
R³ is hydrogen,
R⁴ is hydrogen or fluorine,
R^{5a} is fluorine or methyl,
R^{5b} is hydrogen,
and the salts thereof, the solvates thereof and the solvates of the salts thereof.

Preference is also given to compounds of the formula (I) in which
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl or 1H-indazol-6-yl,
   where the 5-membered heterocycle in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by 1 to 2 substituents selected independently from the group consisting of oxo, methyl, ethyl and n-propyl,
      in which ethyl and n-propyl may be substituted by 4 to 7 fluorine substituents,
      or
      in which ethyl and n-propyl are substituted by a hydroxycarbonyl substituent and in which ethyl and n-propyl are additionally substituted by 4 to 6 fluorine substituents,
   and
   where the benzyl ring in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by a substituent selected from the group consisting of fluorine and chlorine,
R² is ethyl, isopropyl, cyclopropyl, cyclobutyl, cyclohexyl, or heterocyclyl bonded via a carbon atom, selected from the group of pyrrolidinyl and piperidinyl,
   where ethyl is substituted by a trifluoromethyl substituent,
   and
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo and methyl,
R³ is hydrogen,
R⁴ is hydrogen or fluorine,
R^{5a} is fluorine, chlorine or methyl,
R^{5b} is hydrogen,
and the salts thereof, the solvates thereof and the solvates of the salts thereof.

Preference is also given to compounds of the formula (I) in which
R¹ is 2,3-dihydro-1H-benzimidazol-5-yl, 2,3-dihydro-1,3-benzoxazol-5-yl, 2,3-dihydro-1H-indazol-6-yl, 1H-benzimidazol-6-yl or 1H-indazol-6-yl,
   where the 5-membered heterocycle in 2,3-dihydro-1H-benzimidazol-5-yl, 2,3-dihydro-1,3-benzoxazol-5-yl, 2,3-dihydro-1H-indazol-6-yl, 1H-benzimidazol-6-yl or 1H-indazol-6-yl may be substituted by 1 to 2 substituents selected independently from the group consisting of oxo and methyl,
   and
   where the benzyl ring in 2,3-dihydro-1H-benzimidazol-5-yl, 2,3-dihydro-1,3-benzoxazol-5-yl, 2,3-dihydro-1H-indazol-6-yl, 1H-benzimidazol-6-yl and 1H-indazol-6-yl may be substituted by a chlorine substituent,
R² is ethyl, isopropyl, cyclopropyl or cyclobutyl,
   where ethyl is substituted by a trifluoromethyl substituent,
R³ is hydrogen,
R⁴ is hydrogen,
R^{5a} is chlorine or methyl,
R^{5b} is hydrogen,
and the salts thereof, the solvates thereof and the solvates of the salts thereof.

Preference is also given to compounds of the formula (I) in which

R¹ is a group of the formula

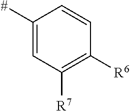

where # is the attachment site to the nitrogen atom,
R⁶ is triazolyl or tetrazolyl,
   where triazolyl may be substituted by a substituent selected from the group consisting of methyl, ethyl and n-propyl,
   in which ethyl and n-propyl are substituted by a hydroxycarbonyl substituent and in which ethyl and n-propyl are additionally substituted by 4 to 6 fluorine substituents,
and
R⁷ is hydrogen.

Preference is also given to compounds of the formula (I) in which

R¹ is a group of the formula

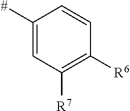

where # is the attachment site to the nitrogen atom,
R⁶ is triazolyl,
   where triazolyl may be substituted by a substituent selected from the group consisting of methyl, ethyl and n-propyl,
      in which ethyl and n-propyl are substituted by a hydroxycarbonyl substituent and in which ethyl and n-propyl are additionally substituted by 4 to 6 fluorine substituents,
and
R⁷ is hydrogen.

Preference is also given to compounds of the formula (I) in which

R¹ is a group of the formula

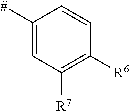

where # is the attachment site to the nitrogen atom,
R⁶ is tetrazolyl, and
R⁷ is hydrogen.

Preference is also given to compounds of the formula (I) in which

R¹ is a group of the formula

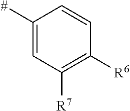

where # is the attachment site to the nitrogen atom,
R⁶ is triazolyl,
   where triazolyl may be substituted by a substituent selected from the group consisting of methyl, ethyl and n-propyl,
      in which ethyl and n-propyl are substituted by a substituent selected from the group consisting of hydroxycarbonyl, C₁-C₄-alkoxycarbonyl, aminocarbonyl and C₁-C₃-alkylaminocarbonyl, and in which ethyl and n-propyl are additionally substituted by 4 to 6 fluorine substituents,
and
R⁷ is hydrogen.

Preference is also given to compounds of the formula (I) in which
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl or 1H-indazol-6-yl,
   where the 5-membered heterocycle in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by 1 to 2 substituents selected independently from the group consisting of oxo, methyl, ethyl and n-propyl,
      in which ethyl and n-propyl may be substituted by 4 to 7 fluorine substituents,
      or
      in which ethyl and n-propyl are substituted by a hydroxycarbonyl substituent and in which ethyl and n-propyl are additionally substituted by 4 to 6 fluorine substituents,
   and
   where the benzyl ring in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by a substituent selected from the group consisting of fluorine, chlorine and hydroxycarbonyl.

Preference is also given to compounds of the formula (I) in which
R¹ is 2,3-dihydro-1H-benzimidazol-5-yl, 2,3-dihydro-1,3-benzoxazol-5-yl, 2,3-dihydro-1H-indazol-6-yl, 1H-benzimidazol-6-yl or 1H-indazol-6-yl,
   where the 5-membered heterocycle in 2,3-dihydro-1H-benzimidazol-5-yl, 2,3-dihydro-1,3-benzoxazol-5-yl, 2,3-dihydro-1H-indazol-6-yl, 1H-benzimidazol-6-yl and 1H-indazol-6-yl may be substituted by 1 to 2 substituents selected independently from the group consisting of oxo and methyl,
   and
   where the benzyl ring in 2,3-dihydro-1H-benzimidazol-5-yl, 2,3-dihydro-1,3-benzoxazol-5-yl, 2,3-dihydro-1H-indazol-6-yl, 1H-benzimidazol-6-yl and 1H-indazol-6-yl may be substituted by a chlorine substituent.

Preference is also given to compounds of the formula (I) in which
R¹ is 2,3-dihydro-1H-benzimidazol-5-yl, 2,3-dihydro-1H-indazol-6-yl, 1H-benzimidazol-6-yl or 7-fluoro-2,3-dihydro-1,3-benzoxazol-5-yl,
   where 2,3-dihydro-1H-benzimidazol-5-yl, 2,3-dihydro-1H-indazol-6-yl, 1H-benzimidazol-6-yl and 7-fluoro-2,3-dihydro-1,3-benzoxazol-5-yl may be substituted by 1 to 2 substituents selected independently from the group consisting of oxo and methyl.

Preference is also given to compounds of the formula (I) in which
R² is hydrogen, C₁-C₄-alkyl, cyclopropyl, cyclohexyl, or heterocyclyl bonded via a carbon atom, selected from the group of pyrrolidinyl, piperidinyl, 3-azabicyclo[3.1.0]hex-6-yl and 8-azabicyclo[3.2.1]oct-3-yl,
   where alkyl may be substituted by 1 to 2 substituents selected independently from the group consisting of amino and C₁-C₃-alkylamino,
   and
   where cyclohexyl may be substituted by a substituent selected from the group consisting of amino and C₁-C₃-alkylamino,
   and
   where pyrrolidinyl, piperidinyl, 3-azabicyclo[3.1.0]hex-6-yl and 8-azabicyclo[3.2.1]oct-3-yl may be substituted by 1 to 2 substituents independently selected from the group consisting of oxo, fluorine, C₁-C₄-alkyl and 2,2,2-trifluoroeth-1-yl.

Preference is also given to compounds of the formula (I) in which
R² is ethyl, isopropyl, cyclopropyl, cyclobutyl, cyclohexyl, or heterocyclyl bonded via a carbon atom, selected from the group of pyrrolidinyl and piperidinyl,
   where ethyl is substituted by a trifluoromethyl substituent,
   and
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo and methyl.

Preference is also given to compounds of the formula (I) in which
R² is cyclohexyl, or heterocyclyl bonded via a carbon atom, selected from the group of pyrrolidinyl and piperidinyl,
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo and methyl.

Preference is also given to compounds of the formula (I) in which
R² is cyclohexyl,
   where cyclohexyl is substituted by a substituent selected from the group consisting of hydroxyl, amino and C₁-C₃-alkylamino.

Preference is also given to compounds of the formula (I) in which
R² is heterocyclyl bonded via a carbon atom, selected from the group of pyrrolidinyl and piperidinyl,
   where pyrrolidinyl and piperidinyl may be substituted by 1 to 2 substituents independently selected from the group consisting of oxo and methyl.

Preference is also given to compounds of the formula (I) in which R³ is hydrogen.

Preference is also given to compounds of the formula (I) in which R² and R³ together with the nitrogen atom to which they are bonded form a pyrrolidinyl or piperazinyl.

Preference is also given to compounds of the formula (I) in which R⁴ is hydrogen or fluorine.

Preference is also given to compounds of the formula (I) in which R⁴ is hydrogen.

Preference is also given to compounds of the formula (I) in which R^{5a} is hydrogen, chlorine, methyl and methoxy and RSb is hydrogen.

Preference is also given to compounds of the formula (I) in which R^{5a} is fluorine or methyl and R^{5b} is hydrogen.

Preference is also given to compounds of the formula (I) in which R^{5a} is methyl and R^{5b} is hydrogen.

The individual radical definitions specified in the particular combinations or preferred combinations of radicals are, independently of the particular combinations of the radicals specified, also replaced as desired by radical definitions of other combinations.

Very particular preference is given to combinations of two or more of the abovementioned preferred ranges.

Preference is also given to
3-[5-(4-{[(2S)-2-({[trans-4-(aminomethyl)cyclohexyl]carbonyl}amino)-3-{2′-methyl-4′-[(1-methylpiperidin-4-yl)carbamoyl]biphenyl-4-yl}propanoyl]amino}phenyl)-1H-1,2,4-triazol-3-yl]-2,2,3,3-tetrafluoropropanoic acid hydrochloride
or
3-[5-(4-{[(2S)-2-({[trans-4-(aminomethyl)cyclohexyl]carbonyl}-amino)-3-{4′-[(trans-4-hydroxycyclohexyl)carbamoyl]-2′-methylbiphenyl-4-yl}propanoyl]amino}-phenyl)-4H-1,2,4-triazol-3-yl]-2,2,3,3-tetrafluoropropanoic acid hydrochloride (enantiomer 1)
or
3-[5-(4-{[(2S)-2-({[trans-4-(aminomethyl)cyclohexyl]carbonyl}-amino)-3-{4′-[(trans-4-hydroxycyclohexyl)carbamoyl]-2′-methylbiphenyl-4-yl}propanoyl]-amino}phenyl)-4H-1,2,4-triazol-3-yl]-2,2,3,3-tetrafluoropropanoic acid (enantiomer 1)
or
3-[5-(4-{[(2S)-2-({[trans-4-(aminomethyl)cyclohexyl]carbonyl}amino)-3-(2′-methyl-4′-{[(3R)-2-oxopiperidin-3-yl]carbamoyl}biphenyl-4-yl)propanoyl]amino}phenyl)-4H-1,2,4-triazol-3-yl]-2,2,3,3-tetrafluoropropanoic acid hydrochloride,
or one of the salts, the solvates or the solvates of the salts of these compounds.

Particular preference is also given to 3-[5-(4-({[(2S)-2-({[trans-4-(aminomethyl)cyclohexyl]carbonyl}-amino)-3-{4′-[(trans-4-hydroxycyclohexyl)carbamoyl]-2′-methylbiphenyl-4-yl}propanoyl]amino}-phenyl)-4H-1,2,4-triazol-3-yl]-2,2,3,3-tetrafluoropropanoic acid hydrochloride (enantiomer 1) having the following formula

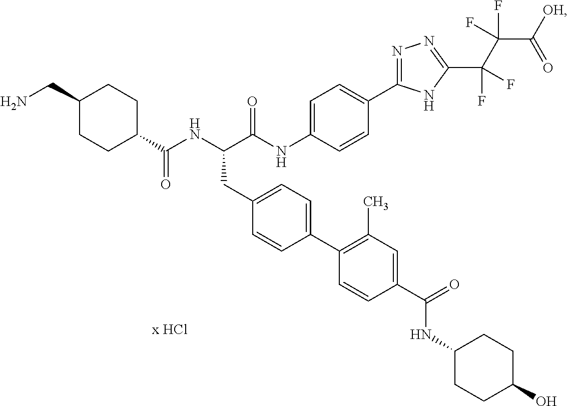 [SMILES: Cc1cc(C(=O)N[C@H]2CC[C@H](O)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

Particular preference is also given to 3-[5-(4-({[(2S)-2-({[trans-4-(aminomethyl)cyclohexyl]carbonyl}-amino)-3-{4′-[(trans-4-hydroxycyclohexyl)carbamoyl]-2′-methylbiphenyl-4-yl}propanoyl]-amino}phenyl)-4H-1,2,4-triazol-3-yl]-2,2,3,3-tetrafluoropropanoic acid (enantiomer 1) having the following formula

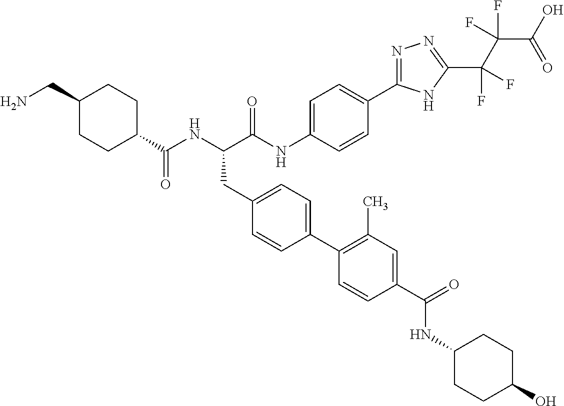 [SMILES: Cc1cc(C(=O)N[C@H]2CC[C@H](O)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

or one of the salts thereof, solvates thereof or solvates of the salts thereof.

Preference is also given to
4′-{(2S)-2-({[trans-4-(aminomethyl)cyclohexyl]carbonyl}amino)-3-oxo-3-[(2-oxo-2,3-dihydro-1H-benzimidazol-5-yl)amino]propyl}-2-methyl-N-[(2S)-1,1,1-trifluoropropan-2-yl]biphenyl-4-carboxamide
or
4′-{(2S)-2-({[trans-4-(aminomethyl)cyclohexyl]carbonyl}amino)-3-[(1-methyl-2-oxo-2,3-dihydro-1H-benzimidazol-5-yl)amino]-3-oxopropyl}-N-isopropyl-2-methylbiphenyl-4-arboxamide hydrochloride
or
4′-{(2S)-2-({[trans-4-(aminomethyl)cyclohexyl]carbonyl}amino)-3-oxo-3-[(3-oxo-2,3-dihydro-1H-indazol-6-yl)amino]propyl}-N-cyclobutyl-2-methylbiphenyl-4-carboxamide hydrochloride or
4′-[(2S)-2-({([trans-4-(aminomethyl)cyclohexyl]carbonyl}amino)-3-(1H-indazol-6-ylamino)-3-oxopropyl]-N-cyclopropyl-2-methylbiphenyl-4-carboxamide hydrochloride
or
4′-{(2S)-2-({[trans-4-(aminomethyl)cyclohexyl]carbonyl}amino)-3-[(7-chloro-2-oxo-2,3-dihydro-1,3-benzoxazol-5-yl)amino]-3-oxopropyl}N-cyclobutyl-2-methylbiphenyl-4-carboxamide hydrochloride
or one of the salts, the solvates or the solvates of the salts of these compounds.

The invention further provides 2,2,3,3-tetrafluoro-3-[5-(4-nitrophenyl)-1H-1,2,4-triazol-3-yl]propanoic acid having the following formula

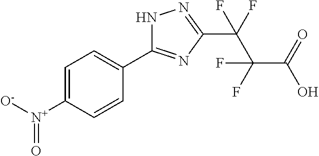 [SMILES: O=C(O)C(F)(F)C(F)(F)c1n[nH]c(-c2ccc([N+](=O)[O-])cc2)n1]

or

3-[5-(4-aminophenyl)-1H-1,2,4-triazol-3-yl]-2,2,3,3-tetrafluoropropanoic acid hydrochloride having the following formula

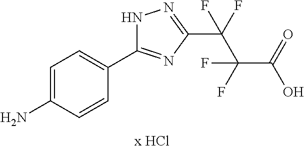 [SMILES: Nc1ccc(-c2nc(C(F)(F)C(F)(F)C(=O)O)n[nH]2)cc1]

or

methyl 2,2,3,3-tetrafluoro-3-[5-(4-nitrophenyl)-1H-1,2,4-triazol-3-yl]propanoate having the following formula

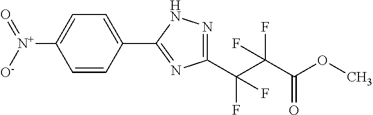 [SMILES: COC(=O)C(F)(F)C(F)(F)c1n[nH]c(-c2ccc([N+](=O)[O-])cc2)n1]

or

methyl 3-[5-(4-aminophenyl)-1H-1,2,4-triazol-3-yl]-2,2,3,3-tetrafluoropropanoate having the following formula

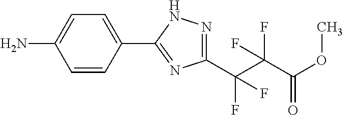 [SMILES: COC(=O)C(F)(F)C(F)(F)c1n[nH]c(-c2ccc(N)cc2)n1]

or

3-[5-(4-aminophenyl)-1H-1,2,4-triazol-3-yl]-2,2,3,3-tetrafluoro-N,N-dimethylpropanamide having the following formula

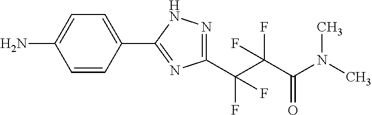 [SMILES: CN(C)C(=O)C(F)(F)C(F)(F)c1n[nH]c(-c2ccc(N)cc2)n1]

or

3-[5-(4-aminophenyl)-1H-1,2,4-triazol-3-yl]-2,2,3,3-tetrafluoropropanamide having the following formula

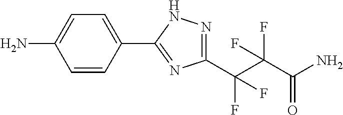 [SMILES: NC(=O)C(F)(F)C(F)(F)c1n[nH]c(-c2ccc(N)cc2)n1]

or

3-[5-(4-aminophenyl)-1H-1,2,4-triazol-3-yl]-2,2,3,3-tetrafluoro-N-methylpropanamide having the following formula

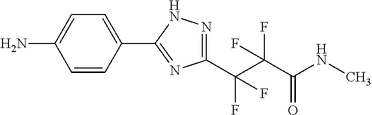 [SMILES: CNC(=O)C(F)(F)C(F)(F)c1n[nH]c(-c2ccc(N)cc2)n1]

or

3-[5-(4-aminophenyl)-1H-1,2,4-triazol-3-yl]-2,2,3,3-tetrafluoropropanoic acid having the following formula

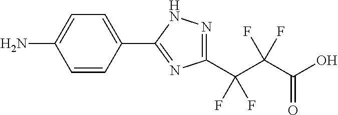 [SMILES: Nc1ccc(-c2nc(C(F)(F)C(F)(F)C(=O)O)n[nH]2)cc1]

or one of the salts, the solvates or the solvates of the salts of these compounds.

The invention further provides 2,2,3,3-tetrafluoro-3-[5-(4-nitrophenyl)-1H-1,2,4-triazol-3-yl]propanoic acid having the following formula

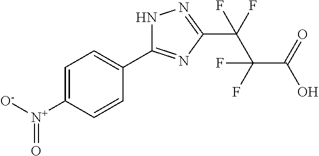 [SMILES: O=C(O)C(F)(F)C(F)(F)c1n[nH]c(-c2ccc([N+](=O)[O-])cc2)n1]

or

3-[5-(4-aminophenyl)-1H-1,2,4-triazol-3-yl]-2,2,3,3-tetrafluoroprpanoic acid hydrochloride having the following formula

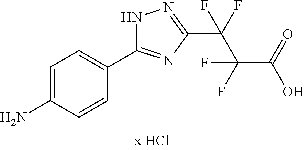 [SMILES: Nc1ccc(-c2nc(C(F)(F)C(F)(F)C(=O)O)n[nH]2)cc1]

or

2,2,3,3,4,4-hexafluoro-4-[5-(4-nitrophenyl)-4H-1,2,4-triazol-3-yl]butanoic acid having the following formula

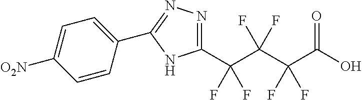 [SMILES: O=C(O)C(F)(F)C(F)(F)C(F)(F)c1nnc(-c2ccc([N+](=O)[O-])cc2)[nH]1]

or

4-[5-(4-aminophenyl)-4H-1,2,4-triazol-3-yl]-2,2,3,3,4,4-hexafluorobutanoic acid hydrochloride having the following formula

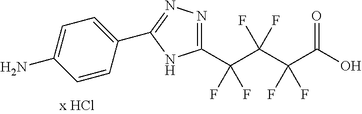 [SMILES: Nc1ccc(-c2nnc(C(F)(F)C(F)(F)C(F)(F)C(=O)O)[nH]2)cc1]

or one of the salts, the solvates or the solvates of the salts of these compounds.

The invention further provides a process for preparing the compounds of the formula (I), or the salts thereof, solvates thereof and the solvates of the salts thereof, wherein the compounds of the formula

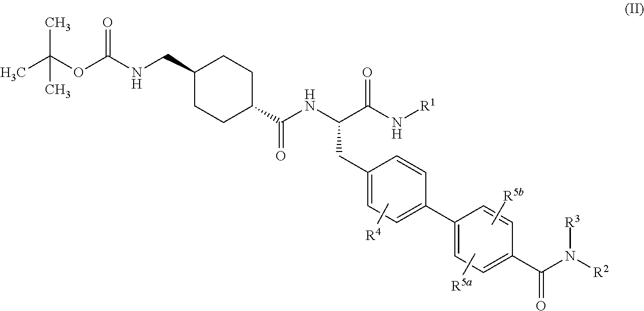

in which

R¹, R², R³, R⁴, R^{5a} and R^{5b} are each as defined above, are reacted with an acid.

The reaction is generally effected in inert solvents, preferably within a temperature range from room temperature to 60° C. at standard pressure.

Inert solvents are, for example, halogenated hydrocarbons such as dichloromethane, trichloromethane, carbon tetrachloride or 1,2-dichloroethane, or ethers such as tetrahydrofuran or dioxane, preference being given to dioxane.

Acids are, for example, trifluoroacetic acid or hydrogen chloride in dioxane, preference being given to hydrogen chloride in dioxane.

The compounds of the formula (II) are known or can be prepared by reacting

[A] Compounds of the Formula

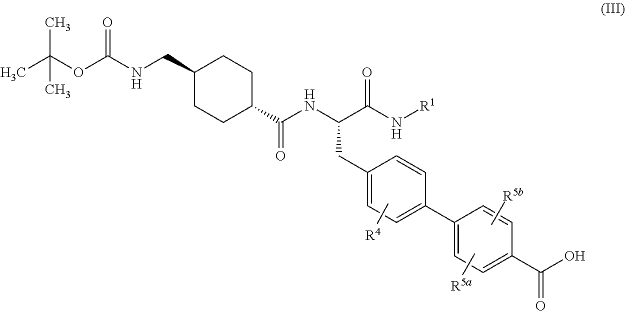

in which

R¹, R⁴, R^{5a} and R^{5b} are each as defined above

with compounds of the formula

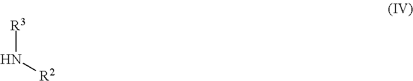

in which

R² and R³ have the meaning given above,

in the presence of a dehydrating reagent,

or

[B] Compounds of the Formula

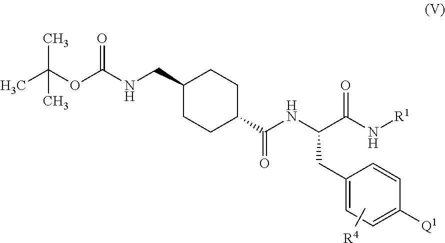

in which

R¹ and R⁴ have the meaning given above and

Q¹ is —B(OH)₂, a boronic ester, preferably pinacol boronate, or —BF₃⁻K⁺, with compounds of the formula

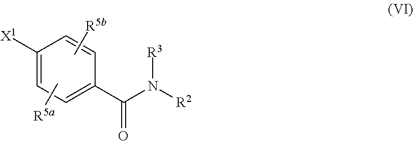

in which

R², R³, R^{5a} and R^{5b} are each as defined above and

X¹ is bromine or iodine,

under Suzuki coupling conditions,

or

[C] Compounds of the Formula

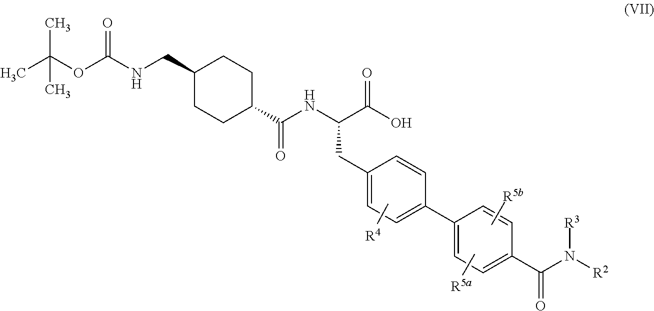

in which

R², R³, R⁴, R^{5a} and R^{5b} are each as defined above,

is reacted with compounds of the formula

H₂N—R¹ (VIII)

in which

R¹ has the meaning given above

in the presence of a dehydrating reagent.

The reaction in process [A] is generally effected in inert solvents, optionally in the presence of a base, preferably within a temperature range from 0° C. to the reflux of the solvents at standard pressure.

Suitable dehydrating reagents here are, for example, carbodiimides, for example N,N′-diethyl-, N,N′-dipropyl-, N,N′-diisopropyl- and N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1, 1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′N′-tetramethyluronium hexafluorophosphate (HATU), or 1-hydroxybenzotriazole (HOBt), or benzotriazol-1-yloxytris(dimethylamino)phosphonium hexafluorophosphate (BOP), or ethyl cyano(hydroxyimino)acetate (Oxyma), or (1-cyano-2-ethoxy-2-oxoethylideneaminooxy) dimethylaminomorpholinocarbenium hexafluorophosphate (COMU), or N-[(dimethylamino)(3H-[1,2,3]triazolo[4,5-b]pyridin-3-yloxy)methylidene]-N-methylmethanaminium hexafluorophosphate, or 2,4,6-tripropyl-1,3,5,2,4,6-trioxatriphosphinane 2,4,6-trioxide (T3P), or mixtures of these, preference being given to N-[(dimethylamino)(3H-[1,2,3]triazolo[4,5-b]pyridin-3-yloxy)methylidene]-N-methylmethanaminium hexafluorophosphate or 2,4,6-tripropyl-1,3,5,2,4,6-trioxatriphosphinane 2,4,6-trioxide (T3P).

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine, preference being given to diisopropylethylamine.

Inert solvents are, for example, halogenated hydrocarbons such as dichloromethane or trichloromethane, hydrocarbons such as benzene, or other solvents such as nitromethane, tetrahydrofuran, dioxane, dimethylformamide, dimethyl sulphoxide, acetonitrile or pyridine, or mixtures of the solvents, preference being given to tetrahydrofuran or dimethylformamide or a mixture of dimethylformamide and pyridine.

The compounds of the formula (IV) are known, can be synthesized from the corresponding starting compounds by known processes or can be prepared analogously to the processes described in the Examples section.

The reaction in process [B] is generally effected in inert solvents, in the presence of a catalyst, optionally in the presence of an additional reagent, optionally in a microwave, preferably within a temperature range from room temperature to 150° C. at standard pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphineferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)], preference being given to tetrakistriphenylphosphinepalladium(0), [1,1-bis-(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)].

Additional reagents are, for example, potassium acetate, caesium carbonate, potassium carbonate or sodium carbonate, potassium tert-butoxide, caesium fluoride or potassium phosphate, which may be present in aqueous solution; preferred additional reagents are those such as potassium acetate or a mixture of potassium acetate and sodium carbonate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, or mixtures of the solvents with alcohols such as methanol or ethanol and/or water, preference being given to toluene, dimethylformamide or dimethyl sulphoxide.

The compounds of the formula (VI) are known, can be synthesized from the corresponding starting compounds by known processes or can be prepared analogously to the processes described in the Examples section.

The reaction in process [C] is effected as described for process [A].

The compounds of the formula (VIII) are known, can be synthesized from the corresponding starting compounds by known processes or can be prepared analogously to the processes described in the Examples section.

The compounds of the formula (III) are known or can be prepared by reacting

[D] compounds of the formula

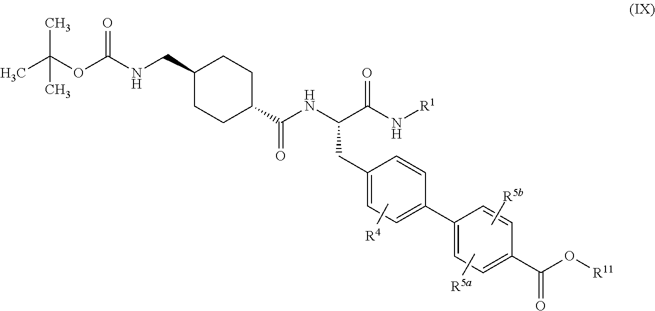

in which

R¹, R⁴, R^{5a} and R^{5b} are each as defined above and

R¹¹ is methyl or ethyl,

with a base,

or

[E] reacting compounds of the formula

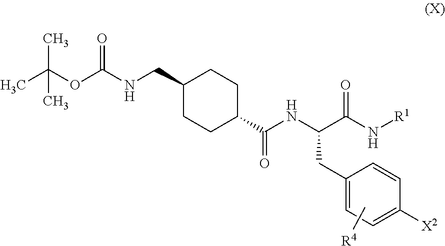

in which

R¹ and R⁴ are each as defined above, and

X² is bromine or iodine,

with compounds of the formula

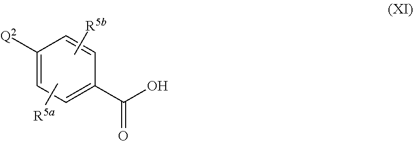

in which

R^{5a} and R^{5b} are each as defined above, and

Q² is —B(OH)₂, a boronic ester, preferably pinacol boronate, or —BF₃⁻K⁺,

under Suzuki coupling conditions.

The reaction in process [D] is generally effected in inert solvents, preferably within a temperature range from room temperature up to the reflux of the solvents at standard pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water, preference being given to a mixture of tetrahydrofuran and water.

Bases are, for example, alkali metal hydroxides such as sodium hydroxide, lithium hydroxide or potassium hydroxide, or alkali metal carbonates such as caesium carbonate, sodium carbonate or potassium carbonate, or alkoxides such as potassium tert-butoxide or sodium tert-butoxide, preference being given to sodium hydroxide and lithium hydroxide.

The reaction in process [E] is effected as described for process [B].

The compounds of the formula (XI) are known, can be synthesized from the corresponding starting compounds by known processes or can be prepared analogously to the processes described in the Examples section.

The compounds of the formula (IX) are known or can be prepared by reacting

[F] compounds of the formula (X) with compounds of the formula

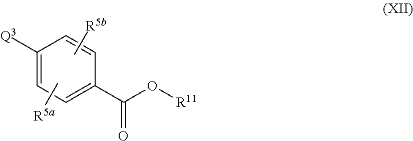

in which

R^{5a} and R^{5b} are each as defined above,

R¹¹ is methyl or ethyl, and

Q³ is —B(OH)₂, a boronic ester, preferably pinacol boronate, or —BF₃⁻K⁺,

under Suzuki coupling conditions,

or

[G] reacting compounds of the formula

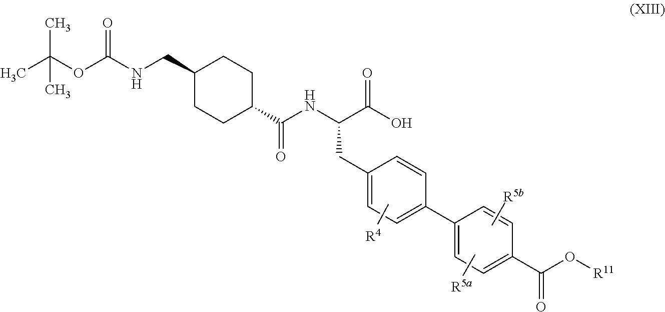

in which

R⁴, R^{5a} and R^{5b} are each as defined above, and

R¹¹ is methyl or ethyl,

with compounds of the formula (VIII) in the presence of a dehydrating reagent.

The reaction in process [F] is effected as described for process [B].

The compounds of the formula (XII) are known, can be synthesized from the corresponding starting compounds by known processes or can be prepared analogously to the processes described in the Examples section.

The reaction in process [G] is effected as described for process [A].

The compounds of the formula (X) are known or can be prepared by reacting compounds of the formula

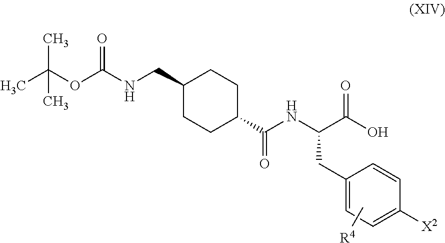

in which

R⁴ is as defined above, and

X² is bromine or iodine,

with compounds of the formula (VIII) in the presence of a dehydrating reagent.

The reaction is effected as described for process [A].

The compounds of the formula (XIV) are known, can be synthesized from the corresponding starting compounds by known processes or can be prepared analogously to the processes described in the Examples section.

The compounds of the formula (XIII) are known or can be prepared by reacting compounds of the formula (XIV) with compounds of the formula (XII) under Suzuki coupling conditions.

The reaction is effected as described for process [B].

The compounds of the formula (V) are known or can be prepared by reacting compounds of the formula (X) with 4,4,4′,4′,5,5,5′,5′-octamethyl-2,2′-bi-1,3,2-dioxaborolane.

The reaction is generally effected in inert solvents, in the presence of a catalyst, optionally in the presence of an additional reagent, optionally in a microwave, preferably within a temperature range from room temperature to 150° C. at standard pressure to 3 bar. Hydrolysis in an acidic medium affords the corresponding boronic acids. Workup with potassium dihydrogenfluoride solution (KHF₂ solution) affords the corresponding trifluoroborates.

Catalysts are, for example, palladium catalysts customary for the borylation of aryl halides, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphineferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)], preference being given to tetrakistriphenylphosphinepalladium(0) and [1,1-bis-(diphenylphosphino)ferrocene]palladium(II) chloride.

Additional reagents are, for example, potassium acetate, caesium carbonate, potassium carbonate or sodium carbonate, potassium tert-butoxide or sodium tert-butoxide, caesium fluoride, potassium phosphate or potassium phenoxide, preference being given to potassium acetate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, preference being given to dioxane, dimethylformamide or dimethyl sulphoxide.

Literature: K. L. Billingslay, T. E. Barde, S. L Buchwald, Angew. Chem. 2007, 119, 5455 or T. Graening, Nachrichten aus der Chemie, January 2009, 57, 34.

The compounds of the formula (VII) are known or can be prepared by reacting compounds of the formula (XIV) with compounds of the formula

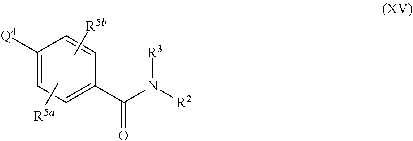

in which

R², R³, R^{5a} and R^{5b} are each as defined above and

Q⁴ is —B(OH)₂, a boronic ester, preferably pinacol boronate, or —BF₃⁻K⁺,

under Suzuki coupling conditions.

The reaction is effected as described for process [B].

The compounds of the formula (XV) are known, can be synthesized from the corresponding starting compounds by known processes or can be prepared analogously to the processes described in the Examples section.

The preparation of the starting compounds and of the compounds of the formula (I) can be illustrated by the synthesis scheme below.

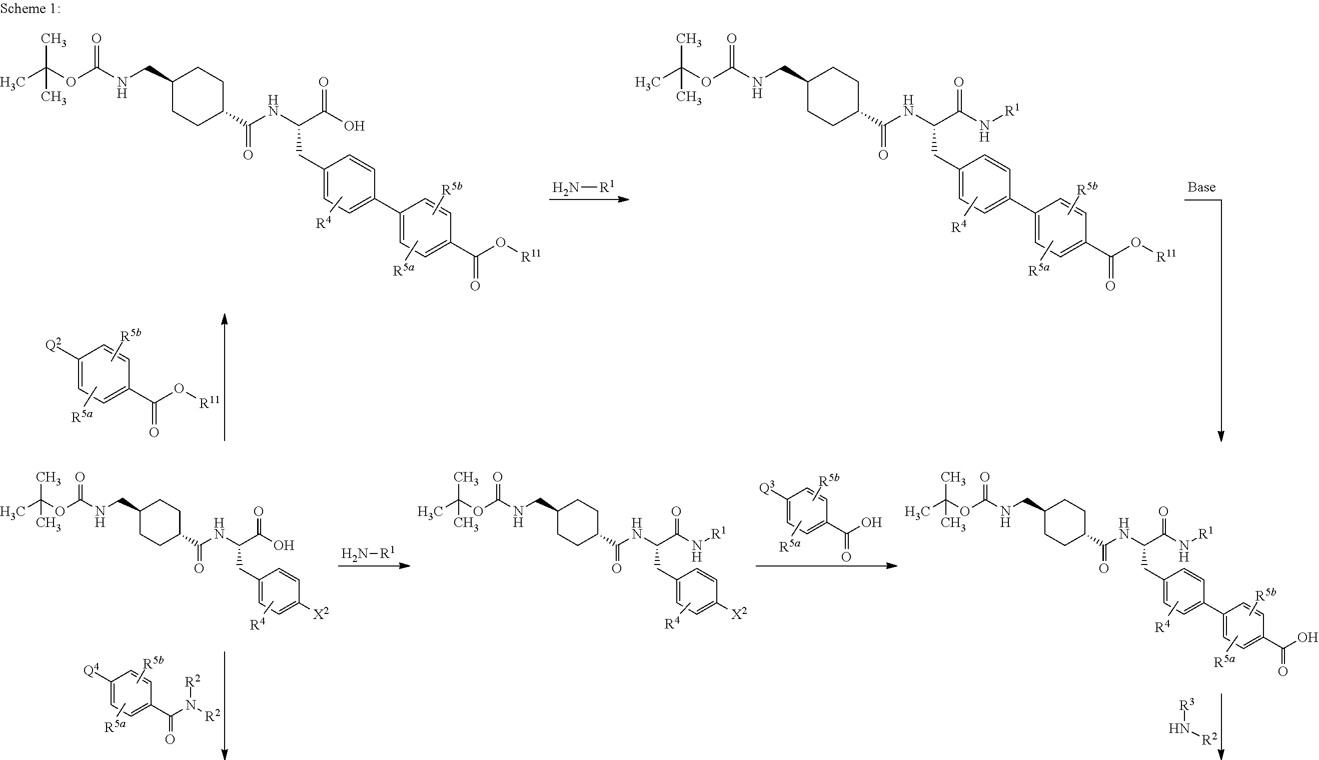

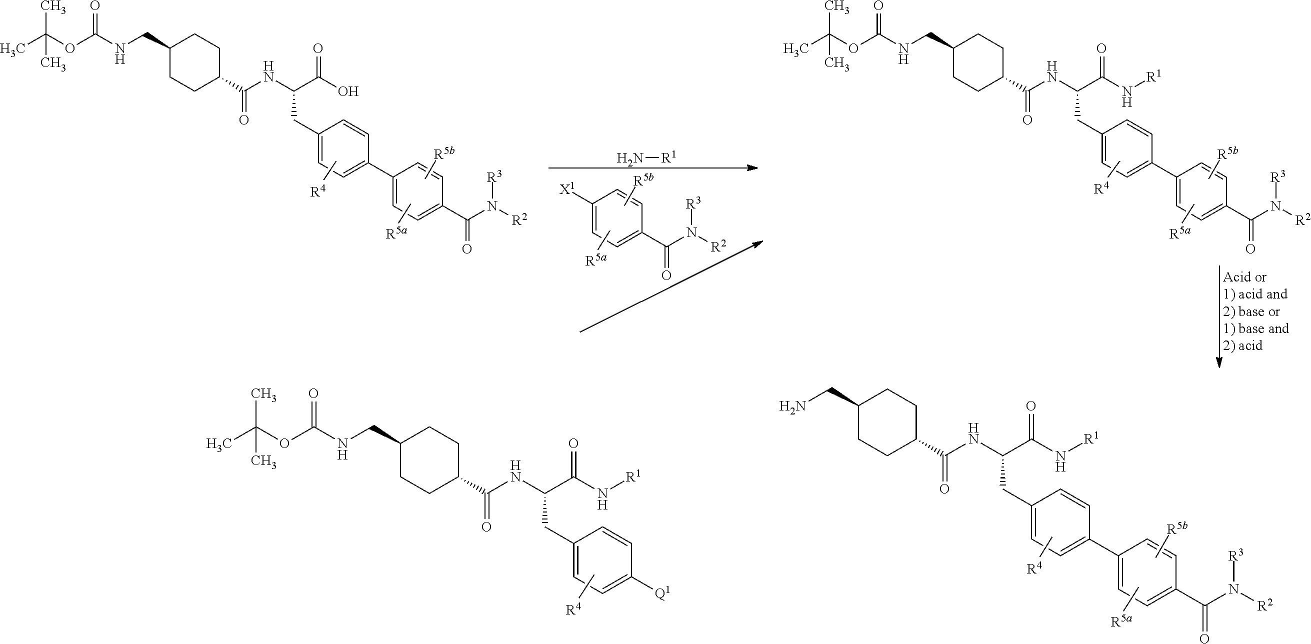

The inventive compounds have an unforeseeable useful spectrum of pharmacological activity and good pharmacokinetic properties. They are compounds that influence the proteolytic activity of the serine proteases FXIa and kallikrein, and possibly plasmin. The inventive compounds inhibit the enzymatic cleavage of substrates that assume a major role in the activation of the blood coagulation cascade and platelet aggregation. If the inventive compounds inhibit plasmin activity, the result is inhibition offibrinolysis.

They are therefore suitable for use as medicaments for treatment and/or prophylaxis of diseases in man and animals.

The present invention further provides for the use of the inventive compounds for treatment and/or prophylaxis of disorders, especially cardiovascular disorders, preferably thrombotic or thromboembolic disorders and/or thrombotic or thromboembolic complications.

“Thromboembolic disorders” in the sense of the present invention include in particular disorders such as acute coronary syndrome (ACS), ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, venous thromboses, especially in deep leg veins and renal veins, transitory ischaemic attacks and also thrombotic and thromboembolic stroke.

The inventive compounds substances are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alia, but also owing to surgical interventions, neoplastic disorders, burns or other injuries and may lead to severe organ damage through microthrombosis.

Thromboembolic complications are also encountered in microangiopathic haemolytic anaemias, extracorporeal circulatory systems, such as haemodialysis, and also prosthetic heart valves.

In addition, the inventive compounds are also used for influencing wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, inflammatory pulmonary disorders, glomerulonephritis and inflammatory intestinal disorders, for example Crohn's disease or ulcerative colitis or acute renal failure, and additionally likewise for the prophylaxis and/or treatment of dementia disorders, for example Alzheimer's disease. In addition, the inventive compounds can be used for inhibiting tumour growth and the formation of metastases, for microangiopathies, age-related macular degeneration, diabetic retinopathy, diabetic nephropathy and other microvascular disorders, and also for the prevention and treatment of thromboembolic complications, for example venous thromboembolisms, for tumour patients, especially those undergoing major surgery or chemo- or radiotherapy.

In addition, the inventive compounds are also suitable for the prophylaxis and/or treatment of pulmonary hypertension.

The term “pulmonary hypertension” includes certain forms of pulmonary hypertension, as determined, for example, by the World Health Organization (WHO). Examples include pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hyptertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for treatment and/or prophylaxis of pulmonary hypertension associated with sarcoidosis, histiocytosis X and lymphangiomatosis.

In addition, the inventive substances may also be useful for treatment of pulmonary and hepatic fibroses.

In addition, the inventive compounds may also be suitable for treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications.

Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. This can be prevented by the inventive compounds. A secondary effect is the consumption of coagulation factors (for example factor X, prothrombin and fibrinogen) and platelets, which reduces the coagulability of the blood and may result in heavy bleeding.

In addition, the inventive compounds are also useful for the prophylaxis and/or treatment of hyperfibrinolysis. The prophylaxis and/or treatment may reduce or eliminate severe perioperative blood loss. Severe bleeding occurs in major operations, for example coronary artery bypass surgery, transplants or hysterectomy, and in the event of trauma, in the event of haemorrhagic shock or in the event of postpartum haemorrhage. In the aforementioned indications, there may be perioperative use of extracorporeal circulation systems or filter systems, for example heart and lung machines, haemofiltration, haemodialysis, extracorporeal membrane oxygenation or a ventricular support system, for example artificial heart. This additionally requires anticoagulation, for which the inventive compounds can also be used.

The inventive compounds are also suitable for anticoagulation during kidney replacement procedures, for example in the case of continuous veno-venous haemofiltration or intermittent haemodialysis.

The inventive compounds can additionally also be used for preventing coagulation ex vivo, for example for preserving blood and plasma products, for cleaning/pretreating catheters and other medical auxiliaries and instruments, for coating synthetic surfaces of medical auxiliaries and instruments used in vivo or ex vivo or for biological samples which could contain factor XIa.

The present invention further provides for the use of the inventive compounds for treatment and/or prophylaxis of disorders, especially the disorders mentioned above.

The present invention further provides for the use of the inventive compounds for production of a medicament for treatment and/or prophylaxis of disorders, especially of the aforementioned disorders.

The present invention further provides a method for treatment and/or prophylaxis of disorders, especially the disorders mentioned above, using a therapeutically effective amount of an inventive compound.

The present invention further provides the inventive compounds for use in a method for treatment and/or prophylaxis of disorders, especially of the aforementioned disorders, using a therapeutically effective amount of an inventive compound.

The present invention further provides medicaments comprising an inventive compound and one or more further active ingredients.

The present invention further provides a method for preventing the coagulation of blood in vitro, especially in banked blood or biological samples which could contain factor XIa, which is characterized in that an anticoagulatory amount of the inventive compound is added.

The present invention further provides medicaments comprising an inventive compound and one or more further active ingredients, especially for treatment and/or prophylaxis of the disorders mentioned above. Preferred examples of active ingredients suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors), for example tissue plasminogen activator (t-PA), streptokinase, reteplase and urokinase;
anticoagulatory substances (anticoagulants), for example heparin (UFH), low-molecular-weight heparins (LMW), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI), for example Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux,
platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), for example abciximab, eptifibatide, tirofiban, lamifiban, lefradafiban and fradafiban;
and also antiarrhythmics;
various antibiotics or antifungal medicaments, either as calculated therapy (prior to the presence of the microbial diagnosis) or as specific therapy;
vasopressors, for example norepinephrine, dopamine and vasopressin;
inotropic therapy, for example dobutamine;
corticosteroids, for example hydrocortisone and fludrocortisone;
recombinant human activated protein C, for example Xigris;
blood products, for example erythrocyte concentrates, thrombocyte concentrates, erythropietin and fresh frozen plasma.

“Combinations” for the purpose of the invention mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

The inventive compounds may act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The inventive compounds can be administered in suitable administration forms for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of an absorption step (for example by an intravenous, intraarterial, intracardiac, intraspinal or intralumbar route) or with inclusion of an absorption (for example by an intramuscular, subcutaneous, intracutaneous, percutaneous or intraperitoneal route). Suitable administration forms for parenteral administration include injection and infusion formulations in the form of solutions, suspensions, emulsions, lyophilizates or sterile powders.

Parenteral administration is preferred.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, non-toxic, pharmaceutically suitable auxiliaries. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for the liquid/liquid solutions are in each case based on volume. “w/v” means “weight/volume”. For example, “10% w/v” means: 100 ml of solution or suspension comprise 10 g of substance.

### A) EXAMPLES

### Abbreviations

bs/br. s. broad singlet (in NMR)
bd broad doublet (in NMR)
cat. catalytic
CI chemical ionization (in MS)
dd doublet of doublets (in NMR)
DMF dimethylformamide
DMSO dimethyl sulphoxide
dt doublet of triplets (in NMR)
of th. of theory (in yield)
EI electron impact ionization (in MS)
eq. equivalent(s)
ESI electrospray ionization (in MS)
h hour(s)
HATU O-(7-azabenzotriazol-1-yl)-N,N,N′N′-tetramethyluronium hexafluorophosphate
HPLC high-pressure high-performance liquid chromatography
LC-MS liquid chromatography-coupled mass spectroscopy
m multiplet (in NMR)
M molar
min minute(s)
MS mass spectrometry
N normal
NMR nuclear magnetic resonance spectrometry
q quartet (in NMR)
quant. quantitative
quint quintet (in NMR)
RT room temperature
Rₜ retention time (in HPLC)
s singlet (in NMR)
TFA trifluoroacetic acid
THF tetrahydrofuran
UV ultraviolet spectrometry

HPLC and LC/MS Methods:

Method 1 (LC-MS):

Instrument: Waters ACQUITY SQD UPLC System; column: Waters Acquity UPLC HSS T3 1.8μ 50 mm×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 210-400 nm.

Method 2 (LC-MS):

Instrument: Micromass Quattro Premier with Waters UPLC Acquity; column: Thermo Hypersil GOLD 1.9μ 50 mm×1 mm; eluent A: 1 l water+0.5 ml 50% formic acid, eluent B: 1 l acetonitrile+0.5 ml 50% formic acid; gradient: 0.0 min 97% A→0.5 min 97% A→3.2 min 5% A→4.0 min 5% A; oven: 50° C.; flow rate: 0.3 ml/min; UV detection: 210 nm.

Method 3 (LC-MS):

Instrument: Waters ACQUITY SQD UPLC System; column: Waters Acquity UPLC HSS T3 1.8μ 30 mm×2 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.60 ml/min; UV detection: 208-400 nm.

Method 4 (LC-MS):

Instrument: Waters Acquity UPLC-MS SQD 3001; column: Acquity UPLC BEH C18 1.7μ 50 mm×2.1 mm; eluent A: water+0.1% formic acid, eluent B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.

Method 5 (LC-MS):

Instrument: Waters Acquity UPLC-MS SQD 3001; column: Acquity UPLC BEH C18 1.7μ 50 mm×2.1 mm; eluent A: water+0.2% ammonia, eluent B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.

Method 6 (HPLC):

System: Labomatic HD-3000 HPLC gradient pump, Labomatic Labocol Vario-2000 fraction collector, column: Chromatorex C-18 125 mm×30 mm, eluent A: 0.1% formic acid in water, eluent B: acetonitrile, gradient: A 95%/B 5%→A 55%/B 45%; flow rate: 150 ml/min; UV detection: 254 nm.

Method 7 (HPLC):

System: Labomatic HD-3000 HPLC gradient pump, Labomatic Labocol Vario-2000 fraction collector, column: Chromatorex C-18 125 mm×30 mm, eluent A: 0.1% formic acid in water, eluent B: acetonitrile; gradient: A 900/B 10%→A 50%/B 50%; flow rate: 150 ml/min; UV detection: 254 nm.

Method 8 (HPLC):

System: Labomatic HD-3000 HPLC gradient pump, Labomatic Labocol Vario-2000 fraction collector, column: Chromatorex C-18 125 mm×30 mm, eluent A: 0.1% formic acid in water, eluent B: acetonitrile; gradient: A 85%/B 15%→A 45%/B 55%; flow rate: 150 ml/min; UV detection: 254 nm.

Method 9 (HPLC):

System: Labomatic HD-3000 HPLC gradient pump, Labomatic Labocol Vario-2000 fraction collector, column: Chromatorex C-18 125 mm×30 mm, eluent A: 0.1% formic acid in water, eluent B: acetonitrile; gradient: A 80%/B 20%→A 40%/B 60%; flow rate: 150 ml/min; UV detection: 254 nm.

Method 10 (HPLC):

Instrument: Waters SQD autopurification system; column: Waters XBridge C18 5μ 100 mm×30 mm; eluent A: water+0.1% formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm.

Method 11 (HPLC):

Instrument: Waters SQD autopurification system; column: Waters XBridge C18 5μ 100 mm×30 mm; eluent A: water+0.2% ammonia (32%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm.

Method 12 (LC-MS):

MS instrument: Waters (Micromass) QM; HPLC instrument: Agilent 1100 series; column: Agilent ZORBAX Extend-C18 3.0 mm×50 mm 3.5 micron; eluent A: 1 l water+0.01 mol ammonium carbonate, eluent B: 1 l acetonitrile; gradient: 0.0 min 98% A→0.2 min 98% A→3.0 min 5% A→4.5 min 5% A; oven: 40° C.; flow rate: 1.75 ml/min; UV detection: 210 nm

Method 13 (LC-MS):

Instrument: Waters ACQUITY SQD UPLC System; column: Waters Acquity UPLC HSS T3 1.8μ 50 mm×1 mm; eluent A: 1 l water+0.25 ml 99% formic acid, eluent B: 1 l acetonitrile+0.25 ml 99% formic acid; gradient: 0.0 min 95% A→6.0 min 5% A→7.5 min 5% A; oven: 50° C.; flow rate: 0.35 ml/min; UV detection: 210-400 nm.

Method 14 (LC-MS):

MS instrument: Waters (Micromass) Quattro Micro; HPLC instrument: Agilent 1100 Series; column: YMC-Triart C18 3μ 50 mm×3 mm; eluent A: 1 l water+0.01 mol ammonium carbonate, eluent B: 1 l acetonitrile; gradient: 0.0 min 100% A→2.75 min 5% A→4.5 min 5% A; oven: 40° C.; flow rate: 1.25 ml/min; UV detection: 210 nm

Method 15 (HPLC):

System: Labomatic HD-3000 HPLC gradient pump, Labomatic Labocol Vario-2000 fraction collector; column: Chromatorex C-18 125 mm×30 mm, eluent A: 0.1% ammonia in water, eluent B: acetonitrile; gradient: A 900/B 10%→A 50%/B 50%; flow rate: 150 ml/min; UV detection: 254 nm.

Method 16 (LC-MS):

MS instrument type: ThermoFisher Scientific LTQ-Orbitrap-XL; HPLC instrument type: Agilent 1200SL; column: Agilent, POROSHELL 120, 3 mm×150 mm, SB-C18 2.7 μm; eluent A: 1 l water+0.1% trifluoroacetic acid; eluent B: 1 l acetonitrile+0.1% trifluoroacetic acid; gradient: 0.0 min 2% B→1.5 min 2% B→15.5 min 95% B→18.0 min 95% B; oven: 40° C.; flow rate: 0.75 ml/min; UV detection: 210 nm.

Method 17 (LC-MS):

MS instrument type: Waters Synapt G2S; UPLC instrument type: Waters Acquity I-CLASS; column: Waters, HSST3, 2.1 mm×50 mm, C18 1.8 μm; eluent A: 1 l water+0.01% formic acid; eluent B: 1 l acetonitrile+0.01% formic acid; gradient: 0.0 min 10% B→0.3 min 10% B→1.7 min 95% B→2.5 min 95% B; oven: 50° C.; flow rate: 1.20 ml/min; UV detection: 210 nm.

Method 18 (LC-MS):

MS instrument type: Waters Synapt G2S; UPLC instrument type: Waters Acquity I-CLASS; column: Waters, HSST3, 2.1 mm×50 mm, C18 1.8 μm; eluent A: 1 l water+0.01% formic acid; eluent B: 1 l acetonitrile+0.01% formic acid; gradient: 0.0 min 10% B→0.3 min 10% B→1.7 min 95% B→2.5 min 95% B; oven: 50° C.; flow rate: 1.20 ml/min; UV detection: 210 nm.

Method 19 (LC-MS):

MS instrument type: ThermoFisher Scientific LTQ-Orbitrap-XL; HPLC instrument type: Agilent 1200SL; column: Agilent, POROSHELL 120, 3 mm×150 mm, SB-C18 2.7 μm; eluent A: 1 l water+0.1% trifluoroacetic acid; eluent B: 1 l acetonitrile+0.1% trifluoroacetic acid; gradient: 0.0 min 2% B→1.5 min 2% B→15.5 min 95% B→18.0 min 95% B; oven: 40° C.; flow rate: 0.75 ml/min; UV detection: 210 nm.

Microwave:

The microwave reactor used was an instrument of the Biotage™ Initiator type.

When inventive compounds are purified by preparative HPLC by the above-described methods in which the eluents contain additives, for example trifluoroacetic acid, formic acid or ammonia, the inventive compounds may be obtained in salt form, for example as trifluoroacetate, formate or ammonium salt, if the inventive compounds contain a sufficiently basic or acidic functionality. Such a salt can be converted to the corresponding free base or acid by various methods known to the person skilled in the art. Weaker salts can be converted to the corresponding chlorides by addition of a little hydrochloride.

If, in the synthesis intermediates and working examples of the invention described below, a compound is given in the form of a salt of the corresponding base or acid, the exact stoichiometric composition of such a salt as obtained by the respective preparation and/or purification process is generally not known. Unless specified in more detail, additions to names and structural formulae, such as “hydrochloride”, “trifluoroacetate”, “sodium salt” or “×HCl”, “×CF₃COOH”, “×Na⁺” are not to be understood stoichiometrically in the case of such salts, but have only descriptive character with regard to the salt-forming components comprised therein.

This applies correspondingly if the synthesis intermediates and working examples or salts thereof were obtained by the preparation and/or purification processes described in the form of solvates, for example hydrates, whose stoichiometric composition (if of a defined type) is not known.

If the starting compounds and examples contain an L-phenylalanine derivative as the central unit, the corresponding stereocentre is described as the (S) configuration. In the absence of further information, there was no check in individual cases as to whether partial epimerization took place in the coupling of the L-phenylalanine intermediate with the amine H₂N—R¹. Thus, a mixture of the inventive compounds of (S) enantiomer and (R) enantiomer may be present. The main component is the (S) enantiomer depicted in each case.

### Starting Compounds

### Example 1A

### Methyl 4-bromo-N-[(trans-4-{[(tert-butoxycarbonyl)amino]methyl}cyclohexyl)carbonyl]-L-phenylalaninate

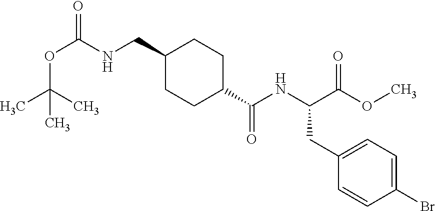

A solution of methyl 4-bromo-L-phenylalaninate (250 g, 874 mmol) and trans-4-{[(tert-butoxycarbonyl)amino]methyl}cyclohexanecarboxylic acid (225 g, 874 mmol) in ethyl acetate (5012 ml) was admixed with N,N-diisopropylethylamine (381 ml, 2186 mmol). The suspension was admixed dropwise with a 2,4,6-tripropyl-1,3,5,2,4,6-trioxatriphosphinane 2,4,6-trioxide solution (500 in dimethylformamide, 766 ml, 1312 mmol) and then the mixture was stirred at RT for 3 h. The reaction mixture was then stirred into water and extracted three times with ethyl acetate. The organic phase was washed with saturated aqueous sodium hydrogencarbonate solution, saturated aqueous ammonium chloride solution, and saturated aqueous sodium chloride solution. The solution was dried over sodium sulphate and the solvent was removed. This gave 420 g (97% of theory) of the title compound.

¹H NMR (400 MHz, DMSO-d₆): δ=0.68-0.92 (m, 2H), 1.04-1.32 (m, 4H), 1.37 (s, 9H), 1.48-1.73 (m, 4H), 2.03 (m, 1H), 2.74 (m, 2H), 2.78-2.90 (m, 1H), 2.94-3.05 (m, 1H), 4.36-4.50 (m, 1H), 6.72-6.85 (m, 1H), 7.17 (d, 2H), 7.46 (d, 2H), 8.15 (d, 1H).

LC-MS (Method 1): Rₜ=1.14 min; MS (ESIpos): m/z=497 [M+H]⁺.

### Example 2A

### 4-Bromo-N-[(trans-4-{[(tert-butoxycarbonyl)amino]methyl}cyclohexyl)carbonyl]-L-phenylalanine

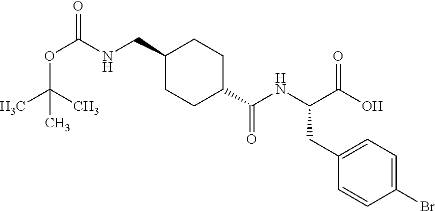

A solution of methyl 4-bromo-N-[(trans-4-{[(tert-butoxycarbonyl)amino]methyl}-cyclohexyl)carbonyl]-L-phenylalaninate in tetrahydrofuran (3000 ml) was admixed with a solution of lithium hydroxide (72 g, 3015 mmol) in water (600 ml). The suspension was stirred at RT for 16 h. The reaction mixture was acidified with 1N hydrochloric acid solution and admixed with ethyl acetate. The organic phase was washed with saturated aqueous sodium chloride solution and dried over sodium sulphate, and the solvent was removed. This gave 284 g (97% of theory) of the title compound.

¹H NMR (400 MHz, DMSO-d₆): δ=0.71-0.90 (m, 2H), 1.22 (d, 4H), 1.37 (s, 9H), 1.45-1.73 (m, 5H), 2.03 (m, 1H), 2.67-2.88 (m, 3H), 2.95-3.09 (m, 1H), 4.38 (m, 1H), 6.77 (s, 1H), 7.17 (d, 2H), 7.46 (d, 2H), 7.99 (d, 1H), 12.65 (br. s, 1H).

LC-MS (Method 1): Rₜ=1.03 min; MS (ESIneg): m/z=481 [M−H]⁻.

### Example 3A

### 4-Bromo-N-[(trans-4-{[(tert-butoxycarbonyl)amino]methyl}cyclohexyl)carbonyl]-3-fluoro-L-phenylalanine

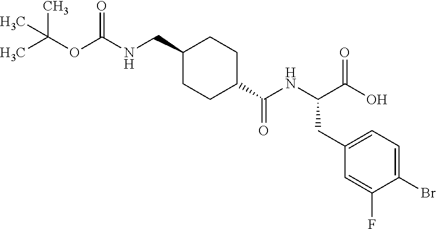

A solution of methyl 4-bromo-3-fluoro-L-phenylalaninate hydrochloride (569 mg, 1.82 mmol) and trans-4-{[(tert-butoxycarbonyl)amino]methyl}cyclohexanecarboxylic acid (562 mg, 2.19 mmol) in ethyl acetate (15 ml) was admixed with N,N-diisopropylethylamine (0.79 ml, 4.55 mmol). The reaction mixture was admixed with a 2,4,6-tripropyl-1,3,5,2,4,6-trioxatriphosphinane 2,4,6-trioxide solution (50% in dimethylformamide, 1.0 ml, 2.19 mmol) and with dimethylformamide until the precipitate dissolved, and then the mixture was stirred at RT for 16 h. The reaction mixture was stirred into ethyl acetate, and washed four times with water and once with saturated aqueous sodium chloride solution. The organic phase was dried over sodium sulphate and the solvent was removed. The residue was stirred with hot acetonitrile and filtered with suction, and the solid was dried under high vacuum. The resulting solid was dissolved in 28 ml of tetrahydrofuran and admixed with a solution of lithium hydroxide monohydrate (472 mg, 11.25 mmol) in water (8 ml). The suspension was stirred at RT for 16 h. The reaction mixture was acidified with 1N hydrochloric acid solution and admixed with ethyl acetate. The phases were separated, the organic phase was washed with water and saturated aqueous sodium chloride solution and dried over sodium sulphate, and the solvent was removed. The residue was recrystallized with a little diethyl ether and then dried under high vacuum. This gave 1048 mg (quant.) of the slightly contaminated title compound over two stages.

¹H NMR (400 MHz, DMSO-d₆): δ=0.71-0.94 (m, 2H), 1.05-1.31 (m, 3H), 1.37 (s, 9H), 1.47-1.56 (m, 1H), 1.60-1.74 (m, 3H), 1.81-1.92 (m, 1H), 1.95-2.15 (m, 1H), 2.69-2.79 (m, 2H), 2.78-2.79 (m, 1H), 2.80-2.90 (m, 1H), 3.01-3.10 (m, 1H), 3.13-3.19 (m, 1H), 4.36-4.46 (m, 1H), 6.74-6.84 (m, 1H), 6.97-7.06 (m, 1H), 7.19-7.26 (m, 1H), 7.26-7.27 (m, 1H), 7.55-7.64 (m, 1H), 7.97-8.06 (m, 1H), 12.0 (br. s, 1H), 12.7 (br. s, 1H).

LC-MS (Method 1): Rₜ=1.05 min; MS (ESIneg): m/z=499 [M−H]⁻.

### Example 4A

### 4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide

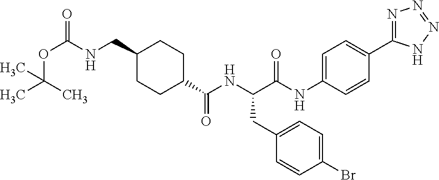

A solution of 4-bromo-N-[(trans-4-{[(tert-butoxycarbonyl)amino]methyl}-cyclohexyl)-carbonyl]-L-phenylalanine (11 g, 22 mmol) and 4-(1H-tetrazol-5-yl)aniline (4 g, 24 mmol) in dimethylformamide (161 ml) was admixed with N,N-diisopropylethylamine (9.6 ml, 55 mmol).

The suspension was admixed dropwise at 0° C. with a 2,4,6-tripropyl-1,3,5,2,4,6-trioxatriphosphinane 2,4,6-trioxide solution (50% in dimethylformamide, 16.9 g, 27 mmol) and then the mixture was stirred at RT for 16 h. The reaction mixture was stirred into ethyl acetate (13000 ml) and extracted three times with water (1570 ml each time). The organic phase was dried with sodium sulphate and the solvent was removed. The crude product was stirred with acetonitrile and filtered off with suction. This gave 11.4 g (78% of theory) of the title compound.

¹H NMR (400 MHz, DMSO-d₆): δ=0.67-0.90 (m, 2H), 1.24 (m, 4H), 1.37 (s, 9H), 1.51-1.74 (m, 4H), 2.02-2.17 (m, 1H), 2.71-2.79 (m, 2H), 2.79-2.89 (m, 1H), 2.99-3.06 (m, 1H), 3.06-3.16 (m, 1H), 3.51-3.67 (m, 1H), 4.55-4.74 (m, 1H), 6.01-6.02 (m, 1H), 6.69-6.84 (m, 1H), 7.21-7.32 (m, 2H), 7.43-7.55 (m, 2H), 7.64-7.76 (m, 2H), 7.88-7.99 (m, 2H), 8.03-8.14 (m, 1H), 10.25 (s, 1H).

LC-MS (Method 1): Rₜ=1.07 min; MS (ESIneg): m/z=624 [M−H]⁻.

### Example 5A

### 4-Bromo-N-alpha-[(trans-4-{[(tert-butoxycarbonyl)amino]methyl}cyclohexyl)carbonyl]-N-[3-fluoro-4-(2H-tetrazol-5-yl)phenyl]-L-phenylalaninamide

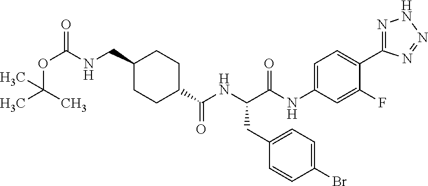

A solution of 4-bromo-N-[(trans-4-{[(tert-butoxycarbonyl)amino]methyl}-cyclohexyl)-carbonyl]-L-phenylalanine (10 g, 20.7 mmol) and 3-fluoro-4-(2H-tetrazol-5-yl)aniline (4.1 g, 22.8 mmol) in ethyl acetate (210 ml) was admixed with N,N-diisopropylethylamine (10.8 ml, 62.1 mmol). Subsequently, 2,4,6-tripropyl-1,3,5,2,4,6-trioxatriphosphinane 2,4,6-trioxide solution (50% in ethyl acetate, 32.9 g, 52 mmol) was added, and the reaction mixture was refluxed for 2 h and then stirred at RT for 48 h. The reaction mixture was admixed with water and the solid formed was filtered off with suction through a frit, washed with ethyl acetate and dried under reduced pressure. This gave 3.97 g (30% of theory) of the title compound.

¹H NMR (300 MHz, DMSO-d₆): δ=0.81 (m, 2H), 1.06-1.29 (m, 3H), 1.36 (s, 9H), 1.46-1.74 (m, 4H), 2.02-2.16 (m, 1H), 2.74 (m, 2H), 2.87 (dd, 1H), 3.00 (dd, 1H), 4.53-4.72 (m, 1H), 6.65-6.79 (m, 1H), 7.24 (d, 2H), 7.39-7.56 (m, 3H), 7.83 (dd, 1H), 8.00 (t, 1H), 8.15 (d, 1H), 10.61 (s, 1H).

LC-MS (Method 4): Rₜ=1.23 min; MS (ESIpos): m/z=645.3 [M+H]⁺.

### Example 6A

### 4-Bromo-N-alpha-[(trans-4-{[(tert-butoxycarbonyl)amino]methyl}cyclohexyl)carbonyl]-3-fluoro-N-[4-(1H-tetrazol-5-yl)phenyl]-L-phenylalaninamide

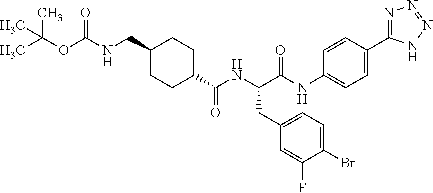

A solution of 4-bromo-N-[(trans-4-{[(tert-butoxycarbonyl)amino]methyl}cyclohexyl)carbonyl]-3-fluoro-L-phenylalanine (1.05 g, 2.09 mmol) and 4-(1H-tetrazol-5-yl)aniline (404 mg, 2.51 mmol) in ethyl acetate (16 ml) was admixed with N,N-diisopropylethylamine (0.91 ml, 5.23 mmol) and stirred at RT for a few minutes. The reaction mixture was admixed with a 2,4,6-tripropyl-1,3,5,2,4,6-trioxatriphosphinane 2,4,6-trioxide solution (50% in dimethylformamide, 1.5 ml, 2.51 mmol) and then the mixture was stirred at RT for 16 h. The reaction mixture was stirred into ethyl acetate, and washed three times with water and once with saturated aqueous sodium chloride solution. The organic phase was dried over sodium sulphate and the solvent was removed. This gave 1.12 g (72% of theory, 87% purity) of the title compound.

¹H NMR (400 MHz, DMSO-d₆): δ=0.74-0.93 (m, 2H), 1.07-1.31 (m, 3H), 1.37 (s, 9H), 1.49-1.59 (m, 1H), 1.61-1.73 (m, 3H), 2.04-2.14 (m, 1H), 2.70-2.78 (m, 2H), 2.83-2.93 (m, 1H), 3.01-3.10 (m, 1H), 4.62-4.72 (m, 1H), 6.74-6.83 (m, 1H), 7.06-7.14 (m, 1H), 7.27-7.33 (m, 1H), 7.59-7.66 (m, 1H), 7.80 (d, 2H), 8.00 (d, 2H), 8.14-8.21 (m, 1H), 10.44 (s, 1H), 16.7 (br. s, 1H).

LC-MS (Method 1): Rₜ=1.10 min; MS (ESIneg): m/z=642 [M−H]⁻.

### Example 7A

### 4-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide

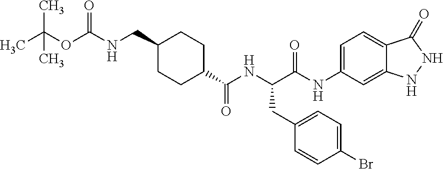

A solution of 4-bromo-N-[(trans-4-{[(tert-butoxycarbonyl)amino]methyl}-cyclohexyl)-carbonyl]-L-phenylalanine (1500 mg, 3 mmol) and 6-amino-1,2-dihydro-3H-indazol-3-one (555 mg, 24 mmol) in ethyl acetate (21 ml) was admixed with N,N-diisopropylethylamine (1.4 ml, 7.8 mmol). The suspension was admixed with a 2,4,6-tripropyl-1,3,5,2,4,6-trioxatriphosphinane 2,4,6-trioxide solution (50% in dimethylformamide, 2.2 ml, 3.7 mmol) and with dimethylformamide until dissolution, and then the mixture was stirred at RT for 16 h. The reaction mixture was stirred into ethyl acetate, and washed twice with water and once with sodium chloride solution. The organic phase was dried with sodium sulphate and the solvent was removed. The crude product was stirred with acetonitrile and filtered off with suction. The residue was separated twice by means of preparative HPLC (eluent: acetonitrile/water gradient, 0.1% TFA). The crude product was stirred with methanol and filtered off with suction. This gave 202 mg (11% of theory) of the title compound.

¹H NMR (400 MHz, DMSO-d₆): δ=0.69-0.89 (m, 2H), 1.04-1.29 (m, 3H), 1.37 (s, 9H), 1.67 (m, 4H), 2.04-2.17 (m, 1H), 2.75 (m, 3H), 2.94-3.07 (m, 1H), 4.54-4.75 (m, 1H), 6.68-6.83 (m, 1H), 6.96 (dd, 1H), 7.25 (d, 2H), 7.39-7.56 (m, 3H), 7.84 (s, 1H), 8.09 (d, 1H), 10.20 (s, 1H), 11.08 (br. s, 1H).

LC-MS (Method 1): Rₜ=1.00 min; MS (ESIpos): m/z=614 [M+H]⁺.

### Example 8A

### 4-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide

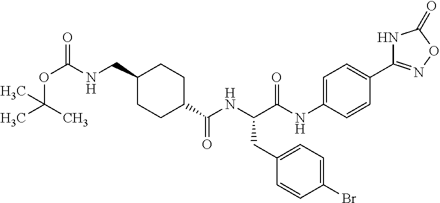

A solution of 4-bromo-N-[(trans-4-{[(tert-butoxycarbonyl)amino]methyl}-cyclohexyl)-carbonyl]-L-phenylalanine (1000 mg, 2 mmol) and 3-(4-aminophenyl)-4,5-dihydro-1,2,4-oxadiazol-5-one (403 mg, 2 mmol) in dimethylformamide (15 ml) was admixed with N,N-diisopropylethylamine (0.9 ml, 5 mmol). The suspension was admixed with a 2,4,6-tripropyl-1,3,5,2,4,6-10 trioxatriphosphinane 2,4,6-trioxide solution (500 in dimethylformamide, 1580 mg, 5 mmol) and with dimethylformamide until dissolution, and then the mixture was stirred at RT for 16 h. The reaction mixture was stirred into ethyl acetate (1200 ml), and washed with water (150 ml) and once with sodium chloride solution. The organic phase was dried with sodium sulphate and the solvent was removed. The crude product was stirred with acetonitrile and filtered off with suction. This gave 540 mg (38% of theory, 94% purity) of the title compound.

¹H NMR (400 MHz, DMSO-d₆): δ=0.68-0.98 (m, 2H), 1.05-1.31 (m, 4H), 1.39 (s, 9H), 1.46-1.76 (m, 4H), 1.98-2.15 (m, 1H), 2.65-3.07 (m, 4H), 4.56-4.71 (m, 1H), 6.71-6.83 (m, 1H), 7.25 (d, 2H), 7.47 (d, 2H), 7.72-7.84 (m, 4H), 8.10-8.20 (m, 1H), 10.45 (s, 1H), 12.86 (br. s, 1H).

LC-MS (Method 1): Rₜ=1.12 min; MS (ESIneg): m/z=640 [M−H]⁻.

### Example 9A

### 4-Bromo-N-alpha-[(trans-4-{[(tert-butoxycarbonyl)amino]methyl}cyclohexyl)carbonyl]-N-1H-indazol-6-yl-L-phenylalaninamide

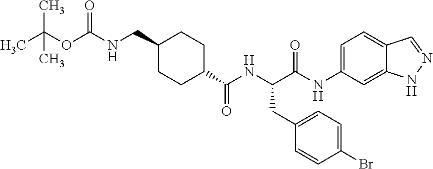

A solution of 4-bromo-N-[(trans-4-{[(tert-butoxycarbonyl)-amino]methyl}-cyclohexyl)-carbonyl]-L-phenylalanine (2000 mg, 4 mmol) and 6-aminoindazole (606 mg, 5 mmol) in dimethylformamide (30 ml) was admixed with N,N-diisopropylethylamine (1.8 ml, 10 mmol). The suspension was admixed with a 2,4,6-tripropyl-1,3,5,2,4,6-trioxatriphosphinane 2,4,6-trioxide solution (50% in dimethylformamide, 3.2 mg, 5 mmol) and with dimethylformamide until dissolution, and then the mixture was stirred at RT for 16 h. The reaction mixture was stirred into ethyl acetate (2500 ml), and washed three times with water (300 ml) and once with sodium chloride solution. The organic phase was dried with sodium sulphate and the solvent was removed. The crude product was stirred with acetonitrile and filtered off with suction. This gave 1400 mg (54% of theory) of the title compound.

¹H NMR (400 MHz, DMSO-d₆): δ=0.68-0.98 (m, 2H), 1.05-1.31 (m, 4H), 1.39 (s, 9H), 1.46-1.76 (m, 4H), 1.98-2.15 (m, 1H), 2.65-3.07 (m, 4H), 4.56-4.71 (m, 1H), 6.71-6.83 (m, 1H), 7.25 (d, 2H), 7.47 (d, 2H), 7.72-7.84 (m, 4H), 8.10-8.20 (m, 1H), 10.45 (s, 1H), 12.86 (br. s, 1H).

LC-MS (Method 1): Rₜ=1.09 min; MS (ESIpos): m/z=598 [M+H]⁺.

### Example 10A

### 4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide

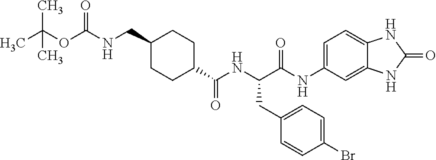

A solution of 4-bromo-N-[(trans-4-{[(tert-butoxycarbonyl)amino]methyl}-cyclohexyl)-carbonyl]-L-phenylalanine (5000 mg, 10 mmol) and 5-amino-1,3-dihydro-2H-benzimidazol-2-one (1851 mg, 12 mmol) in ethyl acetate (70 ml) was admixed with N,N-diisopropylethylamine (4.5 ml, 26 mmol). The suspension was admixed with a 2,4,6-tripropyl-1,3,5,2,4,6-trioxatriphosphinane 2,4,6-trioxide solution (50% in dimethylformamide, 7898 mg, 12 mmol) and with dimethylformamide (20 ml) until dissolution, and then the mixture was stirred at RT for 16 h. The reaction mixture was stirred into ethyl acetate (600 ml), and washed three times with water (300 ml) and once with saturated aqueous sodium chloride solution (250 ml). The precipitate in the organic phase was filtered off and washed with ethyl acetate. The solvent of the filtrate was removed and the residue was dried under high vacuum. This gave 4021 mg (62% of theory) of the title compound.

¹H NMR (400 MHz, DMSO-d₆): δ=0.68-0.89 (m, 2H), 1.17 (m, 3H), 1.37 (s, 9H), 1.66 (m, 4H), 2.02-2.15 (m, 1H), 2.74 (m, 3H), 2.93-3.07 (m, 1H), 3.98-4.09 (dd, 1H), 4.52-4.66 (dd, 1H), 6.72-6.88 (m, 2H), 7.02 (dd, 1H), 7.25 (d, 2H), 7.38-7.53 (m, 3H), 8.10 (d, 1H), 10.04 (s, 1H), 10.51 (s, 1H), 10.59 (s, 1H).

LC-MS (Method 1): Rₜ=1.00 min; MS (ESIneg): m/z=612 [M−H]⁻.

### Example 11A

### Methyl N-[(trans-4-{[(tert-butoxycarbonyl)amino]methyl}cyclohexyl)carbonyl]-4-iodo-L-phenylalaninate

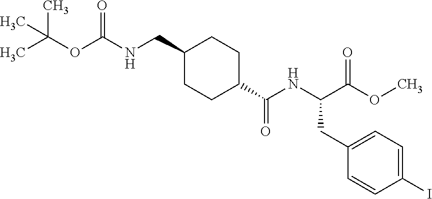

Methyl 4-iodo-L-phenylalaninate hydrochloride (5.7 g, 16.7 mmol), trans-4-{[(tert-butoxycarbonyl)-amino]methyl}cyclohexanecarboxylic acid (4.4 g, 16.7 mmol) and N,N-diisopropylethylamine (11.7 ml, 67 mmol) were suspended in 90 ml of ethyl acetate. The solution was cooled to 0° C. Subsequently, 2,4,6-tripropyl-1,3,5,2,4,6-trioxatriphosphinane 2,4,6-trioxide (50% in ethyl acetate, 26.6 g, 42 mmol) was added dropwise, and the mixture was stirred at 0° C. for 30 minutes and at RT overnight. The mixture was quenched with water and extracted three times with ethyl acetate. The combined organic phases were washed once with saturated aqueous ammonium chloride solution and once with saturated aqueous sodium chloride solution, dried over magnesium sulphate, filtered and concentrated to dryness. The residue was recrystallized from acetonitrile. This gave 5.6 g (73% of theory) of the title compound.

¹H NMR (300 MHz, DMSO-d₆): δ=0.68-0.86 (m, 2H), 1.02-1.27 (m, 3H), 1.33 (s, 9H), 1.45-1.55 (m, 1H), 1.62 (m, 3H), 1.92-2.04 (m, 1H), 2.70 (t, 2H), 2.79 (dd, 1H), 2.94 (dd, 1H), 3.56 (s, 3H), 4.27-4.44 (m, 1H), 6.69-6.79 (m, 1H), 6.98 (d, 2H), 7.59 (d, 2H), 8.10 (d, 1H).

LC-MS (Method 4): Rₜ=1.32 min; MS (ESIpos): m/z=545.2 [M+H]⁺.

### Example 12A

### 4-Bromo-N-alpha-[(trans-4-{[(tert-butoxycarbonyl)amino]methyl}cyclohexyl)carbonyl]-N-(3-chloro-1H-indazol-6-yl)-L-phenylalaninamide

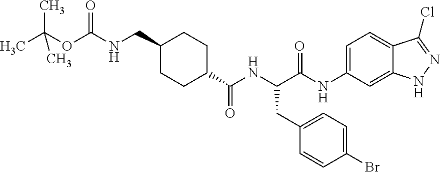

A solution of 4-bromo-N-[(trans-4-{[(tert-butoxycarbonyl)amino]methyl}-cyclohexyl)-carbonyl]-L-phenylalanine (5 g, 10.3 mmol) and 3-chloro-1H-indazol-6-amine (1.9 g, 11.4 mmol) in ethyl acetate (105 ml) was admixed with N,N-diisopropylethylamine (5.4 ml, 31 mmol). Subsequently, 2,4,6-tripropyl-1,3,5,2,4,6-trioxatriphosphinane 2,4,6-trioxide solution (50% in ethyl acetate, 16.5 g, 26 mmol) was added and the mixture was refluxed for 5 h. The reaction mixture was admixed with water and the solid formed was filtered off with suction through a frit, washed with ethyl acetate and dried under reduced pressure. This gave 2.53 g (39% of theory) of the title compound.

¹H NMR (400 MHz, DMSO-d₆): δ=0.72-0.89 (m, 2H), 1.07-1.28 (m, 5H), 1.30-1.40 (m, 13H), 1.42-1.58 (m, 2H), 1.60-1.72 (m, 3H), 2.01-2.14 (m, 1H), 2.69-2.78 (m, 3H), 2.84 (dd, 1H), 3.01 (dd, 1H), 4.59-4.74 (m, 1H), 6.69-6.79 (m, 1H), 7.13-7.21 (m, 1H), 7.24 (d, 2H), 7.42-7.49 (m, 3H), 7.57 (d, 1H), 8.04-8.15 (m, 2H), 10.34 (s, 1H), 13.08 (s, 1H).

LC-MS (Method 4): Rₜ=1.32 min; MS (ESIpos): m/z=634.3 [M+H]⁺.

### Example 13A

### 4-Bromo-N-alpha-[(trans-4-{[(tert-butoxycarbonyl)amino]methyl}cyclohexyl)carbonyl]-N-{4-[3-(trifluoromethyl)-4H-1,2,4-triazol-5-yl]phenyl}-L-phenylalaninamide

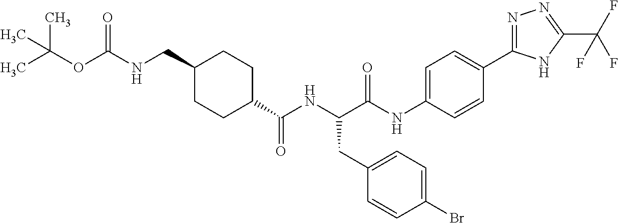

A solution of 4-bromo-N-[(trans-4-{[(tert-butoxycarbonyl)amino]methyl}-cyclohexyl)-carbonyl]-L-phenylalanine (4 g, 8.3 mmol) and 4-[3-(trifluoromethyl)-1H-1,2,4-triazol-5-yl]aniline (2.1 g, 9.1 mmol) in dimethylformamide (105 ml) was admixed with N,N-diisopropylethylamine (3.6 ml, 21 mmol). Subsequently, 2,4,6-tripropyl-1,3,5,2,4,6-trioxatriphosphinane 2,4,6-trioxide solution (50% in dimethylformamide, 6.3 g, 10 mmol) was added and the mixture was stirred at RT overnight. The reaction mixture was admixed with water and the solid formed was filtered off with suction through a frit, washed with a little diethyl ether and water, and dried under reduced pressure. This gave 2.4 g (42% of theory) of the title compound.

¹H NMR (400 MHz, DMSO-d₆): δ=0.74-0.90 (m, 2H), 1.07-1.29 (m, 4H), 1.37 (s, 9H), 1.51-1.59 (m, 1H), 1.68 (m, 3H), 2.03-2.15 (m, 1H), 2.75 (m, 2H), 2.85 (dd, 1H), 3.01 (dd, 1H), 4.58-4.70 (m, 1H), 6.75-6.83 (m, 1H), 7.26 (d, 2H), 7.48 (d, 2H), 7.78 (d, 2H), 7.99 (d, 2H), 8.15 (d, 1H), 10.43 (s, 1H).

LC-MS (Method 1): Rₜ=1.20 min; MS (ESIpos): m/z=695.1 [M+H]⁺.

### Example 14A

### 4-Bromo-N-alpha-[(trans-4-{[(tert-butoxycarbonyl)amino]methyl}cyclohexyl)carbonyl]-N-{4-[3-(difluoromethyl)-1H-1,2,4-triazol-5-yl]phenyl}-L-phenylalaninamide

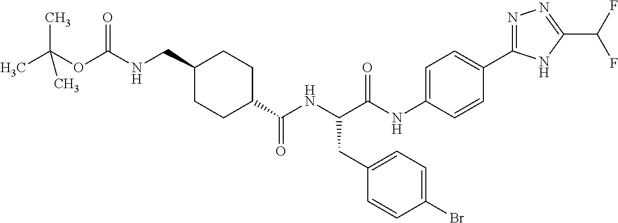

A solution of 4-bromo-N-[(trans-4-{[(tert-butoxycarbonyl)amino]methyl}-cyclohexyl)-carbonyl]-L-phenylalanine (1.82 g, 3.8 mmol) and 4-[3-(difluoromethyl)-1H-1,2,4-triazol-5-yl]aniline (872 mg, 4.15 mmol) in dimethylformamide (27 ml) was admixed with N,N-diisopropylethylamine (1.64 ml, 9.4 mmol). Subsequently, 2,4,6-tripropyl-1,3,5,2,4,6-trioxatriphosphinane 2,4,6-trioxide solution (50% in dimethylformamide, 2.9 g, 4.5 mmol) was added and the mixture was stirred at RT for 96 h. The reaction mixture was admixed with water and the solid formed was filtered off with suction through a frit, washed with water and recrystallized from methanol. This gave 1.5 g (58% of theory) of the title compound.

¹H NMR (400 MHz, DMSO-d₆): δ=0.82 (d, 2H), 1.08-1.29 (m, 3H), 1.37 (s, 9H), 1.55 (m, 1H), 1.68 (m, 3H), 2.00-2.18 (m, 1H), 2.75 (m, 2H), 2.85 (dd, 1H), 3.01 (dd, 1H), 4.65 (m, 1H), 6.79 (t, 1H), 7.26 (d, 2H), 7.48 (d, 2H), 7.76 (d, 2H), 7.97 (d, 2H), 8.15 (d, 1H), 10.40 (s, 1H), 14.82 (br. s., 1H).

LC-MS (Method 1): Rₜ=1.13 min; MS (ESIpos): m/z=677.1 [M+H]⁺.

### Example 15A

### 4-Bromo-N-alpha-[(trans-4-{[(tert-butoxycarbonyl)amino]methyl}cyclohexyl)carbonyl]-N-{4-[3-(pentafluoroethyl)-1H-1,2,4-triazol-5-yl]phenyl}-L-phenylalaninamide

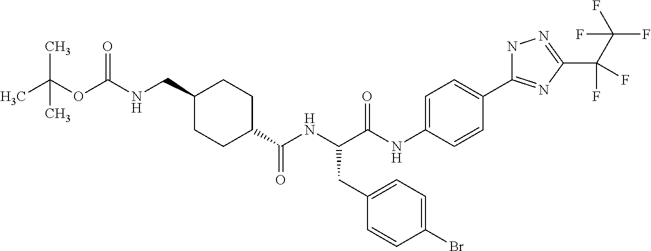

A solution of 4-bromo-N-[(trans-4-{[(tert-butoxycarbonyl)amino]methyl}-cyclohexyl)-carbonyl]-L-phenylalanine (752 mg, 1.6 mmol) and 4-[3-(pentafluoroethyl)-1H-1,2,4-triazol-5-yl]aniline (476 mg, 1.7 mmol) in dimethylformamide (11 ml) was admixed with N,N-diisopropylethylamine (0.68 ml, 3.9 mmol). Subsequently, 2,4,6-tripropyl-1,3,5,2,4,6-trioxatriphosphinane 2,4,6-trioxide solution (50% in dimethylformamide, 1.2 g, 1.9 mmol) was added and the mixture was stirred at RT for 96 h. The reaction mixture was admixed with water and the solid formed was filtered off with suction through a frit, washed with water and recrystallized from methanol. This gave 632 mg (55% of theory) of the title compound.

¹H NMR (400 MHz, DMSO-d₆): δ=0.82 (m, 2H), 1.05-1.29 (m, 3H), 1.37 (s, 9H), 1.55 (d, 1H), 1.68 (m, 3H), 2.02-2.16 (m, 1H), 2.75 (m, 2H), 2.85 (dd, 1H), 3.02 (dd, 1H), 4.65 (m, 1H), 6.79 (t, 1H), 7.26 (d, 2H), 7.48 (d, 2H), 7.78 (d, 2H), 7.99 (d, 2H), 8.15 (d, 1H), 10.43 (s, 1H), 15.28 (br. s., 1H).

LC-MS (Method 1): Rₜ=1.25 min; MS (ESIpos): m/z=745.1 [M+H]⁺.

### Example 16A

### 4-Bromo-N-alpha-[(trans-4-{[(tert-butoxycarbonyl)amino]methyl}cyclohexyl)carbonyl]-N-{4-[3-(heptafluoropropyl)-1H-1,2,4-triazol-5-yl]phenyl}-L-phenylalaninamide

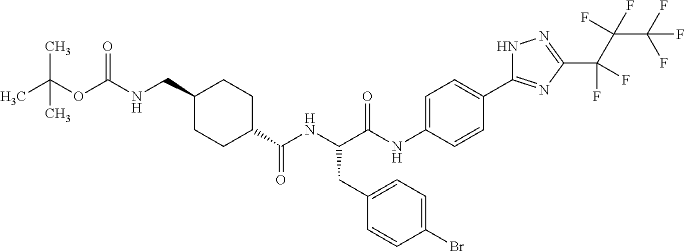

A solution of 4-bromo-N-[(trans-4-{[(tert-butoxycarbonyl)amino]methyl}-cyclohexyl)-carbonyl]-L-phenylalanine (943 mg, 1.95 mmol) and 4-[3-(heptafluoropropyl)-1H-1,2,4-triazol-5-yl]aniline (704 mg, 2.1 mmol) in dimethylformamide (14 ml) was admixed with N,N-diisopropylethylamine (0.85 ml, 4.95 mmol). Subsequently, 2,4,6-tripropyl-1,3,5,2,4,6-trioxatriphosphinane 2,4,6-trioxide solution (50% in dimethylformamide, 1.5 g, 2.34 mmol) was added and the mixture was stirred at RT for 96 h. The reaction mixture was admixed with water and the solid formed was filtered off with suction through a frit, washed with water and recrystallized from methanol. This gave 952 mg (62% of theory) of the title compound.

¹H NMR (400 MHz, DMSO-d₆): δ=0.82 (m, 2H), 1.08-1.29 (m, 3H), 1.37 (s, 9H), 1.50-1.59 (m, 1H), 1.62-1.76 (m, 3H), 2.01-2.19 (m, 1H), 2.75 (m, 2H), 2.85 (dd, 1H), 3.01 (dd, 1H), 4.65 (m, 1H), 6.71-6.87 (m, 1H), 7.26 (d, 2H), 7.48 (d, 2H), 7.78 (d, 2H), 7.99 (d, 2H), 8.15 (d, 1H), 10.43 (s, 1H), 15.30 (br. s., 1H).

LC-MS (Method 1): Rₜ=1.29 min; MS (ESIpos): m/z=795.2 [M+H]⁺.

### Example 17A

### tert-Butyl 5-[4-({4-bromo-N-[(trans-4-{[(tert-butoxycarbonyl)amino]methyl}cyclohexyl)carbonyl]-L-phenylalanyl}amino)phenyl]-3-oxo-2,3-dihydro-1H-pyrazole-1-carboxylate

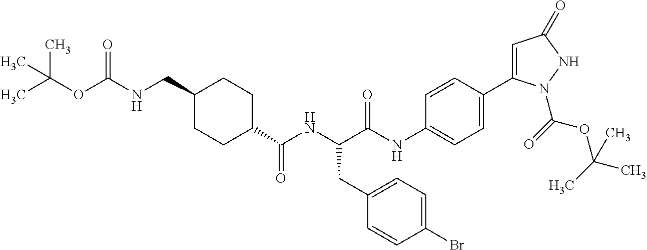

A solution of 134 mg (0.28 mmol) of 4-bromo-N-[(trans-4-{[(tert-butoxycarbonyl)-amino]methyl}-cyclohexyl)carbonyl]-L-phenylalanine and 101 mg (0.33 mmol, 90% purity) of tert-butyl 5-(4-aminophenyl)-3-oxo-2,3-dihydro-1H-pyrazole-1-carboxylate in 2 ml of ethyl acetate was admixed with 0.12 ml (0.69 mmol) of N,N-diisopropylethylamine. The suspension was admixed with 0.19 ml (0.33 mmol) of 2,4,6-tripropyl-1,3,5,2,4,6-trioxatriphosphinane 2,4,6-trioxide solution (50% in dimethylformamide) and with dimethylformamide until dissolution, and then the mixture was stirred at RT for 16 h. The reaction mixture was stirred into ethyl acetate, and washed three times with water and once with sodium chloride solution. The organic phase was dried over sodium sulphate and the solvent was removed. The crude product was dissolved in a little methanol and separated by means of preparative HPLC (eluent: acetonitrile/water with 0.1% TFA (gradient)). This gave 134 mg (64% of theory) of the title compound.

¹H NMR (400 MHz, DMSO-d₆): δ=0.73-0.91 (m, 2H), 1.06-1.32 (m, 3H), 1.37 (s, 9H), 1.45-1.59 (m, 10H), 1.60-1.73 (m, 3H), 2.03-2.14 (m, 1H), 2.70-2.78 (m, 2H), 2.79-2.89 (m, 1H), 2.96-3.07 (m, 1H), 4.59-4.69 (m, 1H), 6.48 (s, 1H), 6.74-6.83 (m, 1H), 7.25 (d, 2H), 7.48 (d, 2H), 7.62-7.73 (m, 4H), 8.13 (d, 1H), 10.27 (s, 1H), 12.95 (s, 1H).

LC-MS (Method 1): Rₜ=1.26 min; MS (ESIneg): m/z=738 [M−H]⁻.

### Example 18A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-4-carboxylate

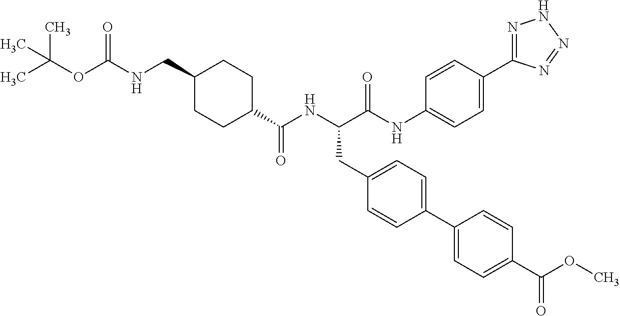

2000 mg (3.19 mmol) of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide and 689 mg (3.81 mmol) of 4-methoxycarbonylphenylboronic acid were taken up in 32 ml of 1,2-dimethoxyethane. After the addition of 130 mg (0.16 mmol) of 1,1′-bis(diphenylphosphino)ferrocenepalladium(II) chloride and 3.2 ml of 2N aqueous sodium carbonate solution, the reaction mixture was stirred under reflux for 2 h and then concentrated. The residue was taken up in acetonitrile, boiled and hot-filtered through a Millipore syringe filter. After cooling to RT, the precipitate was filtered off with suction, washed with a little acetonitrile and dried under high vacuum. This gave 753 mg (34% of theory) of the title compound.

¹H NMR (400 MHz, DMSO-d₆): δ=0.74-0.91 (m, 2H), 1.08-1.30 (m, 3H), 1.36 (s, 9H), 1.50-1.76 (m, 4H), 2.06-2.17 (m, 1H), 2.70-2.78 (m, 2H), 2.88-3.00 (m, 1H), 3.06-3.16 (m, 1H), 3.87 (s, 3H), 4.66-4.77 (m, 1H), 6.73-6.84 (m, 1H), 7.44 (d, 2H), 7.69 (d, 2H), 7.77-7.87 (m, 4H), 7.95-8.06 (m, 4H), 8.20 (d, 1H), 10.48 (s, 1H), 16.7 (br. s, 1H).

LC-MS (Method 1): Rₜ=1.11 min; MS (ESIneg): m/z=680 [M−H]⁻.

### Example 19A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]biphenyl-4-carboxylic acid

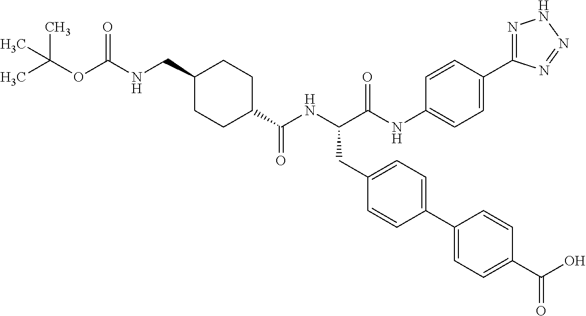

710 mg (1.04 mmol) of methyl 4′-[(2S)-2-{[(trans-4-{[(tert-butoxycarbonyl)amino]methyl}-cyclohexyl)carbonyl]amino}-3-oxo-3-{[4-(2H-tetrazol-5-yl)phenyl]amino}propyl]biphenyl-4-carboxylate were initially charged in 35 ml of tetrahydrofuran, 218 mg (5.21 mmol) of lithium hydroxide monohydrate in 9 ml of water were added and the mixture was stirred at RT for 16 h. The reaction mixture was acidified with 1N hydrochloric acid. After the addition of ethyl acetate, the phases were separated. The organic phase was washed with water and saturated aqueous sodium chloride solution, dried over sodium sulphate, filtered and concentrated to dryness. This gave 437 mg (61% of theory) of the title compound.

¹H NMR (400 MHz, DMSO-d₆): δ=0.74-0.92 (m, 2H), 1.08-1.31 (m, 3H), 1.36 (s, 9H), 1.50-1.76 (m, 4H), 2.06-2.17 (m, 1H), 2.70-2.80 (m, 2H), 2.89-2.99 (m, 1H), 3.06-3.15 (m, 1H), 4.67-4.78 (m, 1H), 6.74-6.81 (m, 1H), 7.43 (d, 2H), 7.68 (d, 2H), 7.80 (dd, 4H), 7.94-8.05 (m, 4H), 8.19 (d, 1H), 10.48 (s, 1H), 13.0 (br. s, 1H), 16.7 (br. s, 1H).

LC-MS (Method 1): Rₜ=0.97 min; MS (ESIneg): m/z=666 [M−H]⁻.

### Example 20A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-carboxylate

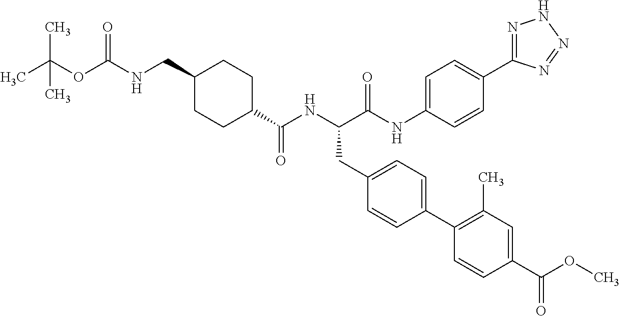

A solution of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide (5000 mg, 7.98 mmol), pinacol 2-methyl-4-methoxycarbonylphenylboronate (4407 mg, 16.0 mmol) and tetrakis(triphenylphosphine)palladium(0) (922 mg, 0.80 mmol) in 1,2-dimethoxyethane (60 ml) and ethanol (25 ml) was admixed with 2N aqueous sodium carbonate solution (15 ml) and the mixture was heated at 100° C. for 16 h. Pinacol 2-methyl-4-methoxycarbonylphenylboronate (1102 mg, 4.0 mmol) and tetrakis(triphenylphosphine)palladium(0) (461 mg, 0.40 mmol) were added and the mixture was heated at 100° C. for 4 h. The reaction mixture was filtered through kieselguhr, and the filtrate was adjusted to pH 1 with 1N hydrochloric acid solution and drawn through silica gel. The mixture was purified by chromatography (silica gel, cyclohexane/ethyl acetate 1:1, then ethyl acetate/ethanol 1:1, then ethanol), and the solvent was removed. This gave 5560 mg (90% of theory, 90% purity) of the title compound.

¹H NMR (400 MHz, DMSO-d₆): δ=0.72-0.91 (m, 2H), 1.09-1.28 (m, 4H), 1.37 (s, 9H), 1.45-1.77 (m, 5H), 2.03-2.17 (m, 1H), 2.25 (s, 3H), 2.89 (m, 1H), 3.08-3.16 (m, 1H), 3.86 (s, 3H), 4.68-4.81 (m, 1H), 6.72-6.84 (m, 1H), 7.25-7.33 (m, 3H), 7.40 (d, 2H), 7.51-7.66 (m, 2H), 7.75 (d, 2H), 7.80-7.84 (m, 1H), 7.87 (s, 1H), 7.97 (d, 2H), 8.17 (d, 1H), 10.36 (br. s, 1H).

LC-MS (Method 1): Rₜ=1.07 min; MS (ESIpos): m/z=697 [M+H]⁺.

### Example 21A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-methylbiphenyl-4-carboxylic acid

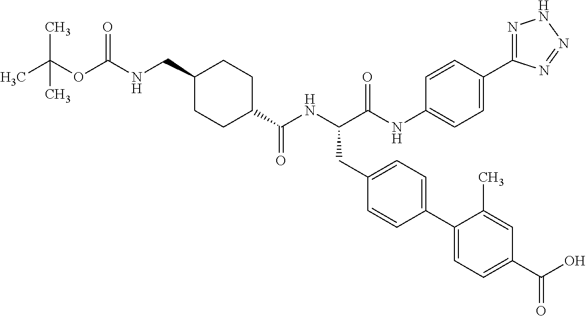

Methyl 4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2-methylbiphenyl-4-carboxylate (3440 mg, 4.4 mmol) was initially charged in a solvent mixture (tetrahydrofuran/water 3:1, 120 ml) and admixed with lithium hydroxide monohydrate (1826 mg, 43 mmol) and stirred at RT for 16 h. Two thirds of the tetrahydrofuran was removed and the solution was acidified to pH 4 with 1N hydrochloric acid solution. The solid formed was filtered off, washed with water and dried under reduced pressure. This gave 2951 mg (94% of theory, 94% purity) of the title compound.

¹H NMR (400 MHz, DMSO-d₆): δ=0.73-0.89 (m, 2H), 1.24 (m, 4H), 1.37 (s, 9H), 1.66 (m, 5H), 2.05-2.16 (m, 1H), 2.23 (s, 3H), 2.75 (m, 2H), 2.91-2.99 (m, 1H), 4.69-4.82 (m, 1H), 6.71-6.80 (m, 1H), 7.21-7.32 (m, 3H), 7.39 (d, 2H), 7.50-7.68 (m, 1H), 7.73-7.89 (m, 4H), 7.98 (d, 2H), 8.15 (d, 1H), 10.37 (s, 1H).

LC-MS (Method 1): Rₜ=0.99 min; MS (ESIneg): m/z=680 [M−H]⁻.

### Example 22A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-chlorobiphenyl-4-carboxylate

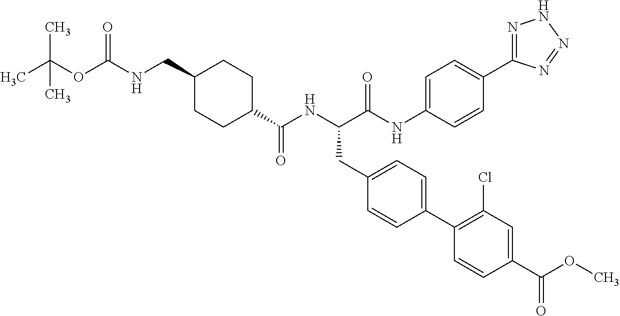

A solution of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide (2350 mg, 3.75 mmol), 2-chloro-4-(methoxycarbonyl)phenylboronic acid (1608 mg, 7.5 mmol) and tetrakis(triphenylphosphine)palladium(0) (433 mg, 0.38 mmol) in 1,2-dimethoxyethane (20 ml) and ethanol (12 ml) was admixed with 2N aqueous sodium carbonate solution (8 ml) and the mixture was heated at 100° C. for 4 h. This was followed by stirring at RT for 16 h. The reaction mixture was filtered through kieselguhr; the filtrate was separated by means of preparative HPLC (eluent: acetonitrile/water with 0.1% TFA (gradient)). The crude product was stirred with acetonitrile and filtered off with suction. This gave 1152 mg (36% of theory, 84% purity) of the title compound.

¹H NMR (400 MHz, DMSO-d₆): δ=0.66-0.92 (m, 2H), 1.06-1.26 (m, 3H), 1.37 (s, 9H), 1.47-1.77 (m, 4H), 2.01-2.19 (m, 1H), 2.69-2.79 (m, 2H), 2.86-3.02 (m, 1H), 3.06-3.22 (m, 1H), 3.89 (s, 3H), 4.64-4.85 (m, 1H), 6.71-6.91 (m, 1H), 7.42 (m, 5H), 7.78-7.88 (m, 2H), 7.93-8.10 (m, 4H), 8.14-8.34 (d, 1H), 10.44 (s, 1H), 16.73 (br. s, 1H).

LC-MS (Method 1): Rₜ=1.16 min; MS (ESIpos): m/z=716 [M+H]⁺.

### Example 23A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-chlorobiphenyl-4-carboxylic acid

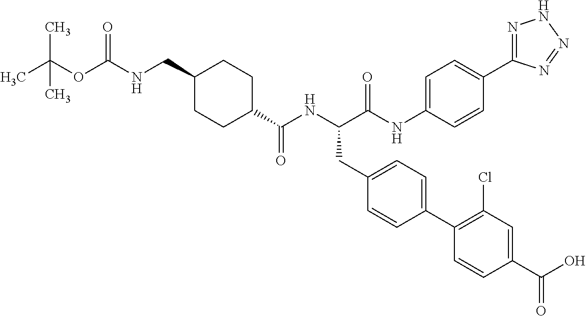

Methyl 4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2-chlorobiphenyl-4-carboxylate (1150 mg, 1.4 mmol) was initially charged in a solvent mixture (tetrahydrofuran/water 3:1, 14 ml) and admixed with lithium hydroxide monohydrate (573 mg, 14 mmol) and stirred at RT for 16 h. The reaction mixture was admixed with water (150 ml) and the solution was acidified to pH 4 with 1N hydrochloric acid solution. The solid formed was filtered off, washed with water and dried under reduced pressure. This gave 1051 mg (100% of theory) of the title compound.

¹H NMR (400 MHz, DMSO-d₆): δ=0.66-0.92 (m, 2H), 1.05-1.30 (m, 4H), 1.37 (s, 9H), 1.51-1.76 (m, 4H), 1.93-2.13 (m, 1H), 2.69-2.78 (m, 2H), 2.83-2.98 (m, 1H), 3.04-3.19 (m, 1H), 4.66-4.83 (m, 1H), 6.69-6.86 (m, 1H), 7.41 (m, 4H), 7.50 (d, 1H), 7.82 (d, 2H), 7.94 (d, 1H), 8.00 (d, 3H), 8.14-8.28 (m, 1H), 10.46 (s, 1H), 13.33 (br. s, 1H), 16.72 (br. s, 1H).

LC-MS (Method 1): Rₜ=0.99 min; MS (ESIneg): m/z=700 [M−H]⁻.

### Example 24A

### Methyl 4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2-methoxybiphenyl-4-carboxylate

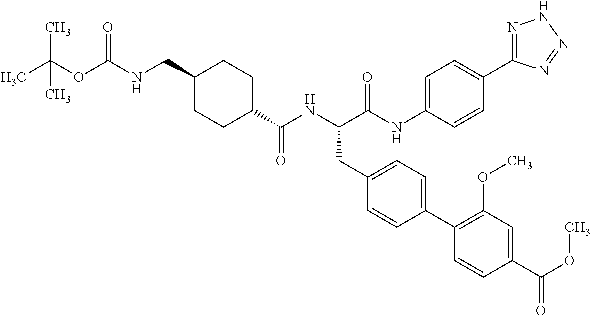

Bis(pinacolato)diborane (1.82 g, 7.2 mmol), methyl 4-bromo-3-methoxybenzoate (1.64 g, 6.7 mmol) and potassium acetate (1.41 g, 14.4 mmol) were initially charged in 37.5 ml of toluene and purged with argon, and [1,1-bis(diphenylphosphino)ferrocene]dichloropalladium-dichloromethane complex (196 mg, 0.24 mmol) was added. The reaction mixture was stirred at 110° C. for 3 h. Then N-alpha-[(trans-4-{[(tert-butoxycarbonyl)amino]methyl}cyclohexyl)carbonyl]-N-[4-(2H-tetrazol-5-yl)phenyl]-L-phenylalaninamide (3000 mg, 4.8 mmol), aqueous sodium carbonate solution (1 g of sodium carbonate in 4.8 ml of water, 9.6 mmol), [1,1-bis(diphenylphosphino)ferrocene]dichloropalladium-dichloromethane complex (121 mg, 0.15 mmol) and ethanol (15 ml) were added. The reaction mixture was stirred at 100° C. for 5 h and at RT overnight, then filtered through kieselguhr, applied to silica gel and purified by chromatography by means of silica gel flash chromatography. The product-containing fractions were combined and concentrated under reduced pressure. This gave 5.1 g (94% of theory) of the title compound.

LC-MS (Method 1): Rₜ=1.11 min; MS (ESIpos): m/z=712.4 [M+H]⁺.

### Example 25A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-methoxybiphenyl-4-carboxylic acid

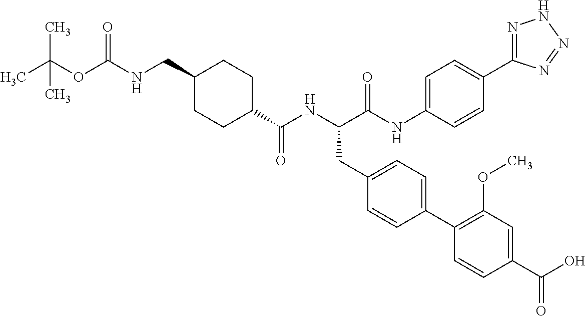

Methyl 4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2-methoxybiphenyl-4-carboxylate (2.5 g, 2.2 mmol) was dissolved in tetrahydrofiuran/water 3/1 (60 ml), lithium hydroxide monohydrate (913 mg, 21.8 mmol) was added and the mixture was stirred at RT overnight. Then the mixture was diluted with ethyl acetate and adjusted to pH 5-6 with 1N hydrochloric acid. The phases were separated and the aqueous phase was extracted with ethyl acetate. The combined organic phases were washed with saturated aqueous sodium chloride solution, dried over sodium sulphate, filtered and concentrated under reduced pressure. This gave 907 mg (57% of theory) of the title compound.

LC-MS (Method 1): Rₜ=0.98 min; MS (ESIpos): m/z=698.3 [M+H]⁺.

### Example 26A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2′-fluoro-2-methylbiphenyl-4-carboxylic acid

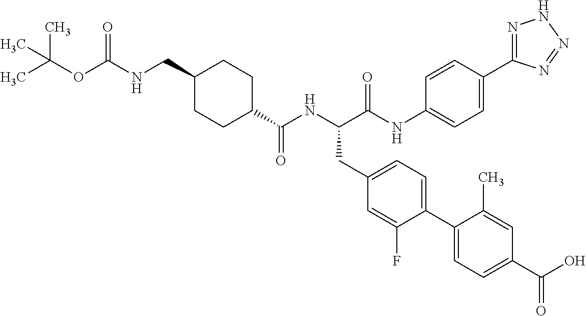

400 mg (0.62 mmol) of 4-bromo-N-alpha-[(trans-4-{[(tert-butoxycarbonyl)amino]methyl}-cyclohexyl)carbonyl]-3-fluoro-N-[4-(2H-tetrazol-5-yl)phenyl]-L-phenylalaninamide and 72 mg (0.06 mmol) of tetrakis(triphenylphosphine)palladium(0) were taken up in 6 ml of 1,2-dimethoxyethane under argon and stirred at RT for 10 min. A solution of 514 mg (1.86 mmol) of methyl 3-methyl-4-(4,4,5,5-tetramethyl-1,3,2-dioxaborolan-2-yl)benzoate in 2 ml of ethanol was added dropwise to the reaction mixture, which was stirred at RT for a further 10 min. After the addition of 5 ml of 2N aqueous sodium carbonate solution, the mixture was stirred at RT for 5 min and under reflux for 3 h. The reaction mixture was admixed with a little methanol, filtered through a Millipore syringe filter and separated twice by means of preparative HPLC (eluent: acetonitrile/water with 0.1% TFA (gradient)). This gave 30 mg (7% of theory, 87% purity) of the title compound and 139 mg of the ester intermediate, which were dissolved in 4 ml of tetrahydrofuran. 40 mg (0.96 mmol) of lithium hydroxide monohydrate in 1.2 ml of water were added to the solution and the mixture was stirred at RT for 16 h. The reaction mixture was admixed with ethyl acetate and with 1N hydrochloric acid to pH 4. The phases were separated, and the organic phase was washed with water and saturated aqueous sodium chloride solution, dried over sodium sulphate, filtered and concentrated to dryness. This gave 103 mg (24% of theory) of the title compound over two stages.

¹H NMR (400 MHz, DMSO-d₆): δ=0.75-0.91 (m, 2H), 1.06-1.31 (m, 3H), 1.37 (s, 9H), 1.49-1.59 (m, 1H), 1.60-1.74 (m, 3H), 2.06-2.17 (m, 4H), 2.70-2.79 (m, 2H), 2.90-3.01 (m, 1H), 3.09-3.19 (m, 1H), 4.71-4.82 (m, 1H), 6.75-6.84 (m, 1H), 7.20-7.32 (m, 3H), 7.82 (d, 3H), 7.86-7.91 (m, 1H), 8.00 (d, 2H), 8.18-8.27 (m, 1H), 10.44 (s, 1H), 13.0 (br. s, 1H), 16.7 (br. s, 1H).

LC-MS (Method 1): Rₜ=1.01 min; MS (ESIneg): m/z=698 [M−H]⁻.

### Example 27A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-3-fluorobiphenyl-4-carboxylate

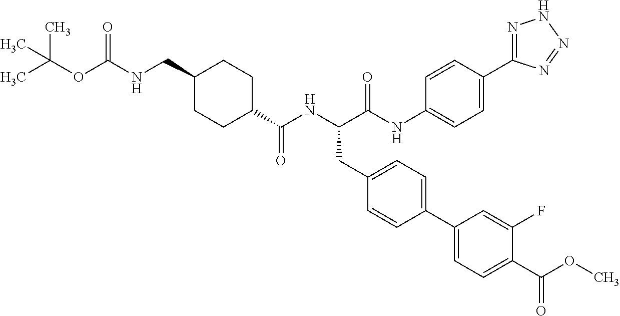

2000 mg (3.19 mmol) of 4-bromo-N-alpha-[(trans-4-{[(tert-butoxycarbonyl)amino]-methyl}cyclohexyl)carbonyl]-N-[4-(2H-tetrazol-5-yl)phenyl]-L-phenylalaninamide and 369 mg (0.32 mmol) of tetrakis(triphenylphosphine)palladium(0) were taken up in 31 ml of 1,2-dimethoxyethane under argon and stirred at RT for 10 min. A solution of 1264 mg (6.38 mmol) of 3-fluoro-4-methoxycarbonylphenylboronic acid in 10.5 ml of ethanol was added dropwise to the reaction mixture and stirred at RT for a further 10 min. After the addition of 26 ml of 2N aqueous sodium carbonate solution, the mixture was stirred at RT for 5 min and under reflux for 3 h. The reaction mixture was filtered through kieselguhr. The residue was separated first by means of flash chromatography (eluent: cyclohexane/ethyl acetate gradient) and then by means of preparative HPLC (eluent: acetonitrile/water with 0.1% TFA (gradient)). This gave 8 mg (0.3% of theory, 93% purity) of the title compound.

LC-MS (Method 1): Rₜ=1.11 min; MS (ESIneg): m/z=698 [M−H]⁻.

### Example 28A

### 4′-[(2S)-2-{[(trans-4-{[(tert-Butoxycarbonyl)amino]methyl}cyclohexyl)carbonyl]amino}-3-{[3-fluoro-4-(2H-tetrazol-5-yl)phenyl]amino}-3-oxopropyl]-2-methylbiphenyl-4-carboxylic acid

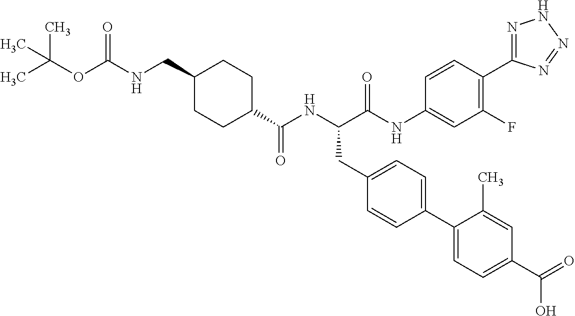

4.39 g (6.8 mmol) of bromo-N-alpha-[(trans-4-{[(tert-butoxycarbonyl)amino]methyl}cyclohexyl)-carbonyl]-N-[3-fluoro-4-(2H-tetrazol-5-yl)phenyl]-L-phenylalaninamide, 787 mg (0.68 mmol) of tetrakis(triphenylphosphine)palladium(0), 1.84 g (10.2 mmol) of 4-(dihydroxyboryl)-3-methylbenzoic acid and 2.2 g (20.4 mmol) of sodium carbonate were taken up in 61 ml of dimethyl sulphoxide and 10 ml of water. The reaction mixture was stirred in the microwave at 110° C. for 6 h. This was followed by addition of plenty of acetonitrile, and the precipitate was filtered off with suction via a frit and dried. This gave 4.7 g (quant.) of the title compound.

LC-MS (Method 1): Rₜ=1.12 min; MS (ESIpos): m/z=700.3 [M+H]⁺.

### Example 29A

### Methyl 4′-{(2S)-2-{[(trans-4-{[(tert-butoxycarbonyl)amino]methyl}cyclohexyl)carbonyl]amino}-3-oxo-3-[(3-oxo-2,3-dihydro-1H-indazol-6-yl)amino]propyl}-2-methylbiphenyl-4-carboxylate

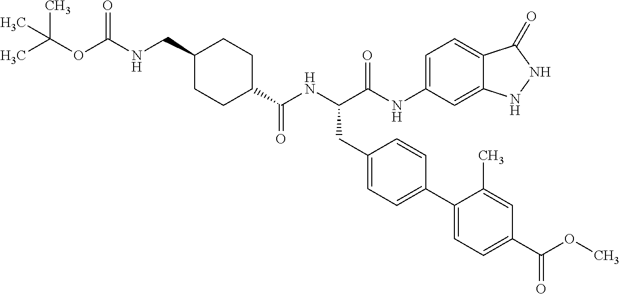

In each of two analogous reactions, 500 mg (0.76 mmol) of N-alpha-[(trans-4-{[(tert-butoxycarbonyl)amino]methyl}cyclohexyl)carbonyl]-4-iodo-N-(3-oxo-2,3-dihydro-1H-indazol-6-yl)-L-phenylalaninamide, 123 mg (0.15 mmol) of 1,1′-bis(diphenylphosphino)ferrocenepalladium(II) chloride and 367 mg (1.22 mmol) of [4-(methoxycarbonyl)-2-methylphenyl]boronic acid were taken up in 6 ml of 1,2-dimethoxyethane and 4 ml of ethanol. After the addition of 2 ml of 2N aqueous sodium carbonate solution, the reaction mixtures were each irradiated in the microwave at 100° C. for 30 min and filtered through kieselguhr, and the combined filtrates were separated by means of column chromatography using silica gel (eluent: ethyl acetate→ethyl acetate/methanol 1:1). The product-containing fractions were concentrated and the residue was stirred with acetonitrile. This gave 971 mg (69% of theory, 86% purity) of the title compound.

¹H NMR (400 MHz, DMSO-d₆): δ=0.68-0.91 (m, 2H), 1.03-1.29 (m, 3H), 1.37 (s, 9H), 1.46-1.56 (m, 1H), 1.57-1.72 (m, 3H), 2.01-2.16 (m, 1H), 2.25 (s, 3H), 2.69-2.80 (m, 2H), 2.86-2.96 (m, 1H), 3.04-3.13 (m, 1H), 3.86 (s, 3H), 4.65-4.75 (m, 1H), 6.73-6.87 (m, 2H), 6.95-7.06 (m, 1H), 7.23-7.33 (m, 3H), 7.38 (d, 2H), 7.44 (s, 1H), 7.78-7.85 (m, 1H), 7.88 (s, 1H), 8.08-8.16 (m, 1H), 9.98 (s, 1H), 10.44-10.64 (m, 2H).

LC-MS (Method 1): Rₜ=1.06 min; MS (ESIpos): m/z=684 [M+H]⁺.

### Example 30A

### 4′-{(2S)-2-{[(trans-4-{[(tert-Butoxycarbonyl)amino]methyl}cyclohexyl)carbonyl]amino}-3-oxo-3-[(3-oxo-2,3-dihydro-1H-indazol-6-yl)amino]propyl}-2-methylbiphenyl-4-carboxylic acid

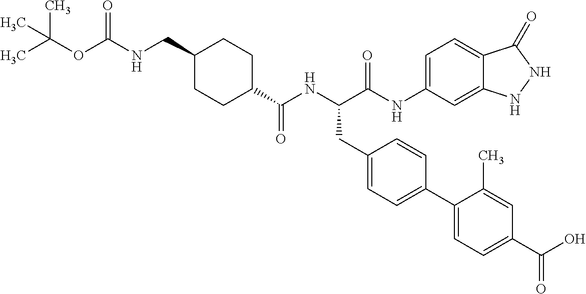

915 mg (1.15 mmol, 86% purity) of methyl 4′-{(2S)-2-{[(trans-4-{[(tert-butoxycarbonyl)-amino]methyl}cyclohexyl)carbonyl]amino}-3-oxo-3-[(3-oxo-2,3-dihydro-1H-indazol-6-yl)-amino]propyl}-2-methylbiphenyl-4-carboxylate were taken up in 12 ml of tetrahydrofuran and 4 ml of water, 483 mg (11.51 mmol) of lithium hydroxide monohydrate were added and the mixture was stirred at RT for 16 h. The reaction mixture was admixed with 50 ml of water and with 1N hydrochloric acid to pH 4. The precipitate formed was filtered off, washed with a little water and then dried under high vacuum. This gave 798 mg (89% of theory, 86% purity) of the title compound.

¹H NMR (400 MHz, DMSO-d₆): δ=0.68-0.92 (m, 2H), 1.03-1.30 (m, 3H), 1.37 (s, 9H), 1.46-1.56 (m, 1H), 1.58-1.73 (m, 3H), 2.03-2.17 (m, 1H), 2.24 (s, 3H), 2.68-2.80 (m, 2H), 2.85-2.97 (m, 1H), 3.02-3.14 (m, 1H), 4.64-4.75 (m, 1H), 6.70-6.89 (m, 2H), 6.96-7.05 (m, 1H), 7.22-7.63 (m, 4H), 7.75-7.89 (m, 2H), 8.04-8.15 (m, 1H), 9.96 (s, 1H), 10.50 (s, 1H), 10.56 (s, 1H), 12.9 (br. s, 1H).

LC-MS (Method 1): Rₜ=0.92 min; MS (ESIpos): m/z=670 [M+H]⁺.

### Example 31A

### 4′-[(2S)-2-{[(trans-4-{[(tert-Butoxycarbonyl)amino]methyl}cyclohexyl)carbonyl]amino}-3-(1H-indazol-6-ylamino)-3-oxopropyl]-2-methylbiphenyl-4-carboxylic acid

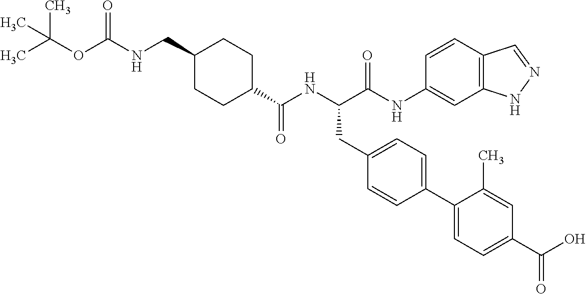

In each of six analogous reactions, 565 mg (0.94 mmol) of 4-bromo-N-alpha-[(trans-4-{[(tert-butoxycarbonyl)amino]methyl}cyclohexyl)carbonyl]-N-1H-indazol-6-yl-L-phenylalaninamide, 69 mg (0.09 mmol) of 1,1′-bis(diphenylphosphino)ferrocenepalladium(II) chloride and 573 mg (2.08 mmol) of 2-methyl-4-methoxycarbonylphenylboronic acid were taken up in 8 ml of 1,2-dimethoxyethane and 3 ml of ethanol. After the addition of 2.5 ml in each case of 2N aqueous sodium carbonate solution, the reaction mixtures were irradiated in the microwave at 120° C. for 1 h, then filtered through kieselguhr, and the combined filtrates were separated by means of column chromatography using silica gel (cyclohexane/ethyl acetate 1:1→100% ethyl acetate). The product-containing fractions were concentrated. The residue was taken up in 90 ml of tetrahydrofuran and 30 ml of water, 1.57 g (37.44 mmol) of lithium hydroxide monohydrate were added and the mixture was stirred at RT for 16 h. Two thirds of the tetrahydrofuran was removed and the reaction mixture was admixed with dichloromethane and 1N hydrochloric acid to pH 4. The precipitated solid was filtered off, washed with dichloromethane and water, and dried under high vacuum. This gave 2.20 g (58% of theory) of the title compound over two stages.

¹H NMR (400 MHz, DMSO-d₆): δ=0.73-0.91 (m, 2H), 1.06-1.31 (m, 3H), 1.37 (s, 9H), 1.48-1.73 (m, 4H), 2.05-2.17 (m, 1H), 2.23 (s, 3H), 2.69-2.79 (m, 2H), 2.89-3.00 (m, 1H), 3.07-3.16 (m, 1H), 4.71-4.82 (m, 1H), 6.74-6.82 (m, 1H), 7.06-7.15 (m, 1H), 7.23-7.31 (m, 3H), 7.39 (d, 2H), 7.67 (d, 1H), 7.80 (d, 1H), 7.83-7.88 (m, 1H), 7.97 (s, 1H), 8.10-8.21 (m, 2H), 10.26 (s, 1H), 12.9 (br. s, 2H).

LC-MS (Method 1): Rₜ=1.01 min; MS (ESIpos): m/z=654 [M+H]⁺.

### Example 32A

### Methyl 4′-{(2S)-2-{[(trans-4-{[(tert-butoxycarbonyl)amino]methyl}cyclohexyl)carbonyl]amino}-3-oxo-3-[(2-oxo-2,3-dihydro-1H-benzimidazol-5-yl)amino]propyl}-2-methylbiphenyl-4-carboxylate

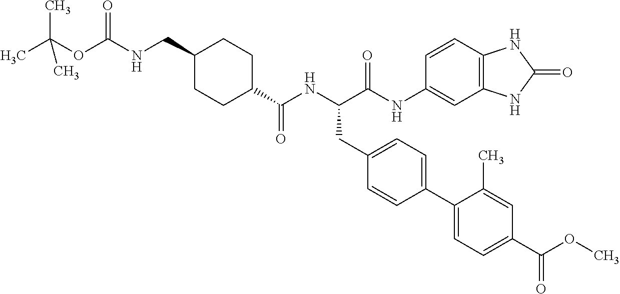

5000 mg (8.4 mmol) of 4-bromo-N-alpha-[(trans-4-{[(tert-butoxycarbonyl)amino]methyl}-cyclohexyl)carbonyl]-N-(2-oxo-2,3-dihydro-1H-benzimidazol-5-yl)-L-phenylalaninamide, 3505 mg (12.7 mmol) methyl 3-methyl-4-(4,4,5,5-tetramethyl-1,3,2-dioxaborolan-2-yl)benzoate and 619 mg (0.84 mmol) of 1,1′-bis(diphenylphosphino)ferrocenepalladium(II) chloride were taken up in 50 ml of 1,2-dimethoxyethane and 30 ml of ethanol. After the addition of 10 ml of 2N aqueous sodium carbonate solution, the mixture was stirred under reflux for 8 h. The salts were filtered off and washed with 1,2-dimethoxyethane. The filtercake was admixed with 30 ml of water and rotated in an ultrasound bath for 2 min. The suspension was filtered, the residue was washed with ethanol and the solvent was removed. The residue was dried under high vacuum. 2498 mg (39% of theory, 89% purity) of the title compound were obtained.

LC-MS (Method 1): Rₜ=1.06 min; MS (ESIpos): m/z=684 [M+H]⁺

### Example 33A

### 4′-{(2S)-2-{[(trans-4-{[(tert-Butoxycarbonyl)amino]methyl}cyclohexyl)carbonyl]amino}-3-oxo-3-[(2-oxo-2,3-dihydro-1H-benzimidazol-5-yl)amino]propyl}-2-methylbiphenyl-4-carboxylic acid

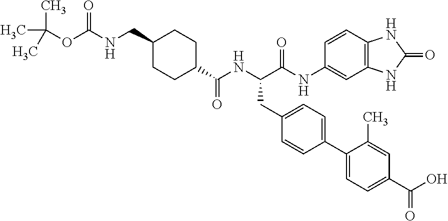

A solution of 2490 mg (3.2 mmol) of methyl 4′-{(2S)-2-{[(trans-4-{[(tert-butoxycarbonyl)-amino]methyl}cyclohexyl)carbonyl]amino}-3-oxo-3-[(2-oxo-2,3-dihydro-1H-benzimidazol-5-yl)amino]propyl}-2-methylbiphenyl-4-carboxylate in 40 ml of tetrahydrofuran/water (3:1) was admixed with 688 mg (17 mmol) of lithium hydroxide. The reaction mixture was stirred at RT for 16 h, about 60% of the tetrahydrofuran was removed, then acidification was effected with 1N hydrochloric acid solution. The solid formed was washed with water and dried under high vacuum. This gave 2402 mg (100% of theory) of the title compound.

LC-MS (Method 1): Rₜ=0.93 min; MS (ESIneg): m/z=668 [M−H]⁻.

### Example 34A

### N-[(trans-4-{[(tert-Butoxycarbonyl)amino]methyl}cyclohexyl)carbonyl]-4-iodo-L-phenylalanine

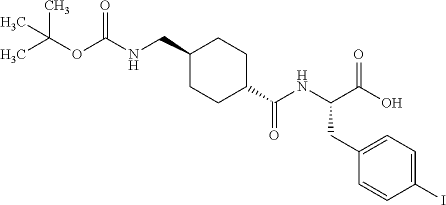

Methyl 4-iodo-N-[(trans-4-{[(tert-butoxycarbonyl)amino]methyl}cyclohexyl)carbonyl]-L-phenylalaninate (3.8 g, 7.0 mmol) was dissolved in 55 ml of tetrahydrofuran, cooled to 0° C. and admixed with 5.3 ml of 2N sodium hydroxide solution. The mixture was allowed to come to RT and stirred at RT overnight. Subsequently, the tetrahydrofuran was drawn off and the aqueous phase was washed twice with tert-butyl methyl ether. The aqueous phase was then adjusted to pH 3 with 1N hydrochloric acid and the precipitated solid was filtered off. The aqueous phase was extracted three times with dichloromethane and the organic phase was concentrated. The residue from the organic phase was combined with the solid and dried under high vacuum. This gave 3.8 g (100% of theory) of the title compound.

¹H NMR (400 MHz, DMSO-d₆): δ=0.72-0.85 (m, 2H), 1.08-1.27 (m, 3H), 1.33 (s, 9H), 1.63 (m, 4H), 1.87-1.96 (m, 1H), 2.70 (t, 2H), 2.83 (dd, 1H), 2.95 (dd, 1H), 3.83 (m, 1H), 6.69-6.75 (m, 1H), 6.84 (d, 2H), 6.93 (d, 1H), 7.47 (d, 2H).

LC-MS (Method 4): Rₜ=1.20 min; MS (ESIpos): m/z=531.1 [M+H]⁺.

### Example 35A

### 4′-{(2S)-2-{[(trans-4-{[(tert-Butoxycarbonyl)amino]methyl}cyclohexyl)carbonyl]amino}-3-[(3-chloro-1H-indazol-6-yl)amino]-3-oxopropyl}-2-methylbiphenyl-4-carboxylic acid

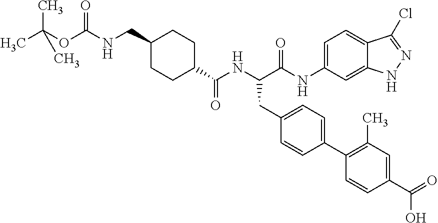

500 mg (0.79 mmol) of 4-bromo-N-alpha-[(trans-4-{[(tert-butoxycarbonyl)amino]methyl}-cyclohexyl)carbonyl]-N-(3-chloro-1H-indazol-6-yl)-L-phenylalaninamide, 91 mg (0.079 mmol) of tetrakis(triphenylphosphine)palladium(0), 311 mg (1.2 mmol) of 4-(dihydroxyboryl)-3-methylbenzoic acid and 251 mg (2.3 mmol) of sodium carbonate were taken up in 6 ml of dimethyl sulphoxide and 1.2 ml of water. The reaction mixture was stirred in the microwave at 110° C. for 90 min. The reaction mixture was converted further without purification.

LC-MS (Method 1): Rₜ=1.21 min; MS (ESIpos): m/z=686.5 [M+H]⁺.

### Example 36A

### Methyl 4′-[(2S)-2-{[(trans-4-{[(tert-butoxycarbonyl)amino]methyl}cyclohexyl)carbonyl]amino}-3-oxo-3-({4-[3-(trifluoromethyl)-1H-1,2,4-triazol-5-yl]phenyl}amino)propyl]-2-methylbiphenyl-4-carboxylate

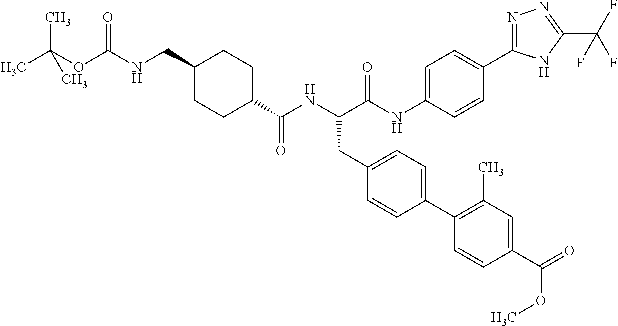

1670 mg (1.85 mmol, 77% purity) of 4-bromo-N-alpha-[(trans-4-{[(tert-butoxycarbonyl)amino]-methyl}cyclohexyl)carbonyl]-N-{4-[3-(trifluoromethyl)-1H-1,2,4-triazol-5-yl]phenyl}-L-phenylalaninamide, 768 mg (2.78 mmol) of methyl 3-methyl-4-(4,4,5,5-tetramethyl-1,3,2-dioxaborolan-2-yl)benzoate and 215 mg (0.185 mmol) of tetrakis(triphenylphosphine)palladium(0) were taken up in 14 ml of 1,2-dimethoxyethane and 6 ml of ethanol. After the addition of 3.4 ml of 2N aqueous sodium carbonate solution, the mixture was stirred under reflux for 5 h and at RT for 48 h. The salts were filtered off through kieselguhr and washed with 1,2-dimethoxyethane. The filtrate was adjusted to pH 6 with 1N hydrochloric acid solution, applied to silica gel and purified by chromatography using silica gel (gradient cyclohexane/ethyl acetate 3/1 to 1/1). The product-containing fractions were concentrated. This gave 1.16 g (67% of theory) of the title compound.

LC-MS (Method 1): Rₜ=1.27 min; MS (ESIpos): m/z=763.4 [M+H]⁺

### Example 37A

### 4′-[(2S)-2-{[(trans-4-{[(tert-Butoxycarbonyl)amino]methyl}cyclohexyl)carbonyl]amino}-3-oxo-3-({4-[3-(trifluoromethyl)-1H-1,2,4-triazol-5-yl]phenyl}amino)propyl]-2-methylbiphenyl-4-carboxylic acid

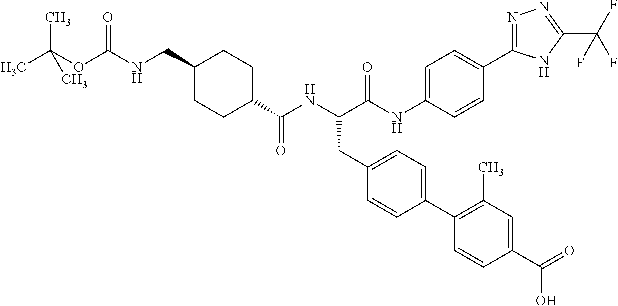

A solution of 1150 mg (1.23 mmol) of methyl 4′-[(2S)-2-{[(trans-4-{[(tert-butoxycarbonyl)-amino]methyl}cyclohexyl)carbonyl]amino}-3-oxo-3-({4-[3-(trifluoromethyl)-1H-1,2,4-triazol-5-yl]phenyl}amino)propyl]-2-methylbiphenyl-4-carboxylate in 30 ml of tetrahydrofuran/water (3:1) was admixed with 519 mg (12.3 mmol) of lithium hydroxide. The reaction mixture was stirred at RT for 16 h, about 60% of the tetrahydrofuran was removed, then acidification was effected with 1N hydrochloric acid solution. The solid formed was filtered off, washed with water and dried under high vacuum. This gave 848 mg (81% of theory, 85% purity) of the title compound.

LC-MS (Method 1): Rₜ=1.12 min; MS (ESIpos): m/z=749.3 [M+H]⁺.

### Example 38A

### Methyl 4′-[(2S)-2-{[(trans-4-{[(tert-butoxycarbonyl)amino]methyl}cyclohexyl)carbonyl]amino}-3-({4-[3-(difluoromethyl)-1H-1,2,4-triazol-5-yl]phenyl}amino)-3-oxopropyl]-2-methylbiphenyl-4-carboxylate

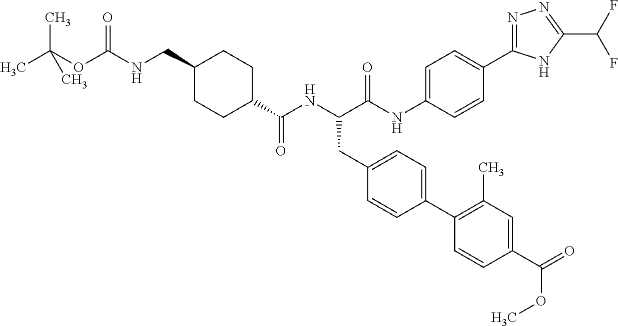

1670 mg (1.85 mmol, 77% purity) of 4-bromo-N-alpha-[(trans-4-{[(tert-butoxycarbonyl)amino]-methyl}cyclohexyl)carbonyl]-N-{4-[3-(difluoromethyl)-1H-1,2,4-triazol-5-yl]phenyl}-L-phenylalaninamide, 768 mg (2.78 mmol) of methyl 3-methyl-4-(4,4,5,5-tetramethyl-1,3,2-dioxaborolan-2-yl)benzoate and 215 mg (0.185 mmol) of tetrakis(triphenylphosphine)palladium(0) were taken up in 14 ml of 1,2-dimethoxyethane and 6 ml of ethanol. After the addition of 3.4 ml of 2N aqueous sodium carbonate solution, the mixture was stirred under reflux for 5 h and at RT for 48 h. The salts were filtered off through kieselguhr and washed with 1,2-dimethoxyethane. The filtrate was adjusted to pH 6 with 1N hydrochloric acid solution, applied to silica gel and purified by chromatography using silica gel (gradient cyclohexane/ethyl acetate 3/1 to 1/1). The product-containing fractions were concentrated. This gave 1.16 g (67% of theory) of the title compound.

LC-MS (Method 1): Rₜ=1.27 min; MS (ESIpos): m/z=763.4 [M+H]⁺

### Example 39A

### 4′-[(2S)-2-{[(trans-4-{[(tert-Butoxycarbonyl)amino]methyl}cyclohexyl)carbonyl]amino}-3-({4-[3-(difluoromethyl)-1H-1,2,4-triazol-5-yl]phenyl}amino)-3-oxopropyl]-2-methylbiphenyl-4-carboxylic acid

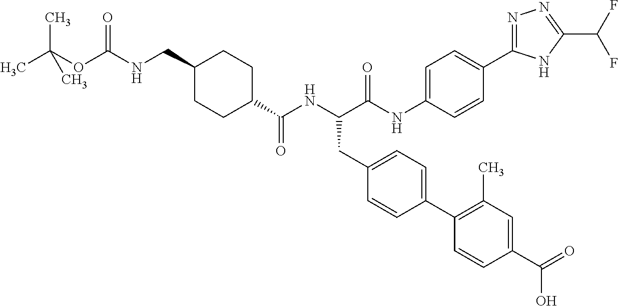

A solution of 475 mg (0.64 mmol) of methyl 4′-[(2S)-2-{[(trans-4-{[(tert-butoxycarbonyl)-amino]methyl}cyclohexyl)carbonyl]amino}-3-({4-[3-(difluoromethyl)-1H-1,2,4-triazol-5-yl]-phenyl}amino)-3-oxopropyl]-2-methylbiphenyl-4-carboxylate in 8 ml of tetrahydrofuran/water (3:1) was admixed with 267 mg (6.4 mmol) of lithium hydroxide. The reaction mixture was stirred at RT for 16 h, about 60% of the tetrahydrofuran was removed, then acidification was effected with 1N hydrochloric acid solution. The solid formed was filtered off, washed with water and dried under high vacuum. This gave 428 mg (90% of theory) of the title compound.

¹H NMR (400 MHz, DMSO-d₆): δ=0.73-0.92 (m, 2H), 1.04-1.29 (m, 4H), 1.37 (s, 9H), 1.49-1.57 (m, 1H), 1.67 (m, 3H), 2.05-2.17 (m, 1H), 2.24 (s, 3H), 2.75 (m, 2H), 2.95 (dd, 1H), 3.10 (dd, 1H), 4.59-4.81 (m, 1H), 6.69-6.86 (m, 1H), 7.21-7.32 (m, 3H), 7.39 (d, 2H), 7.72-7.83 (m, 3H), 7.85 (s, 1H), 7.97 (d, 2H), 8.18 (d, 1H), 10.40 (s, 1H), 12.90 (s, 1H), 14.81 (s, 1H).

LC-MS (Method 1): Rₜ=1.05 min; MS (ESIpos): m/z=731.3 [M+H]⁺

### Example 40A

### Methyl 4′-[(2S)-2-{[(trans-4-{[(tert-butoxycarbonyl)amino]methyl}cyclohexyl)carbonyl]amino}-3-oxo-3-({4-[3-(pentafluoroethyl)-1H-1,2,4-triazol-5-yl]phenyl}amino)propyl]-2-methylbiphenyl-4-carboxylate

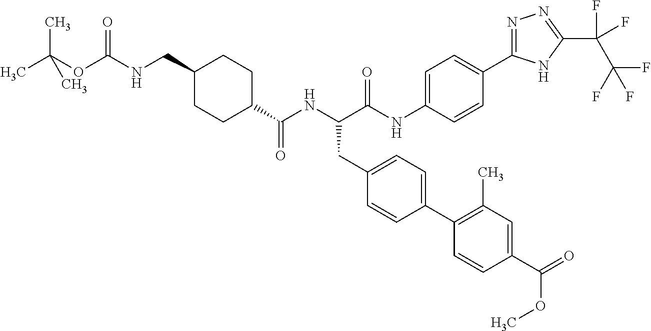

627 mg (0.84 mmol) of 4-bromo-N-alpha-[(trans-4-{[(tert-butoxycarbonyl)amino]methyl}-cyclohexyl)carbonyl]-N-{4-[3-(pentafluoroethyl)-1H-1,2,4-triazol-5-yl]phenyl}-L-phenylalaninamide, 465 mg (1.69 mmol) of methyl 3-methyl-4-(4,4,5,5-tetramethyl-1,3,2-dioxaborolan-2-yl)benzoate and 69 mg (0.084 mmol) of [1,1-bis-(diphenylphosphino)ferrocene]dichloropalladium-dichloromethane complex were taken up in 6 ml of 1,2-dimethoxyethane and 4 ml of ethanol. After the addition of 0.84 ml of 2N aqueous sodium carbonate solution, the mixture was stirred under reflux for 16 h. The reaction mixture was diluted with dimethylformamide, water and acetonitrile, filtered through a Millipore filter and purified by chromatography via HPLC (acetonitrile/water/0.1% trifluoroacetic acid gradient). The product-containing fractions were combined and concentrated. The residue was recrystallized from methanol and acetonitrile. This gave 514 mg (75% of theory) of the title compound.

¹H NMR (400 MHz, DMSO-d₆): δ=0.72-0.92 (m, 2H), 1.08-1.29 (m, 3H), 1.37 (s, 9H), 1.47-1.57 (m, 1H), 1.67 (m, 3H), 2.05-2.17 (m, 1H), 2.25 (s, 3H), 2.74 (m, 2H), 2.95 (dd, 1H), 3.12 (dd, 1H), 3.86 (s, 3H), 4.69-4.79 (m, 1H), 6.72-6.83 (m, 1H), 7.26-7.33 (m, 3H), 7.40 (d, 2H), 7.75-7.84 (m, 3H), 7.88 (s, 1H), 7.99 (d, 2H), 8.18 (d, 1H), 10.43 (s, 1H), 15.28 (s, 1H).

LC-MS (Method 1): Rₜ=1.29 min; MS (ESIpos): m/z=813.4 [M+H]⁺

### Example 41A

### 4′-[(2S)-2-{[(trans-4-{[(tert-Butoxycarbonyl)amino]methyl}cyclohexyl)carbonyl]amino}-3-oxo-3-({4-[3-(pentafluoroethyl)-1H-1,2,4-triazol-5-yl]phenyl}amino)propyl]-2-methylbiphenyl-4-carboxylic acid

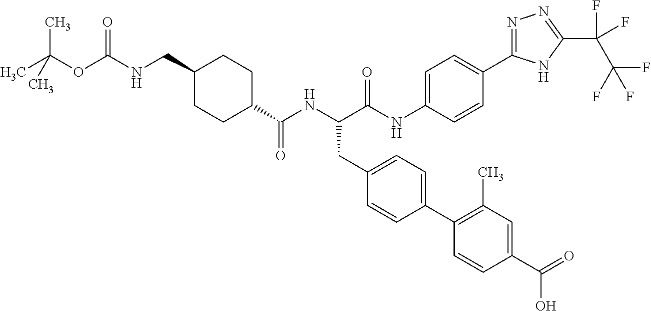

A solution of 509 mg (0.63 mmol) of methyl 4′-[(2S)-2-({[(trans-4-{[(tert-butoxycarbonyl)-amino]-methyl}cyclohexyl)carbonyl]amino}-3-oxo-3-({4-[3-(pentafluoroethyl)-1H-1,2,4-triazol-5-yl]phenyl}amino)propyl]-2-methylbiphenyl-4-carboxylate in 8 ml of tetrahydrofuran/water (3:1) was admixed with 263 mg (6.3 mmol) of lithium hydroxide. The reaction mixture was stirred at RT for 16 h and then at 50° C. for a further 6 h. Subsequently, the mixture was taken up in ethyl acetate and washed with 0.5N hydrochloric acid solution, water and saturated aqueous sodium chloride solution, and the organic phase was dried over sodium sulphate, filtered and concentrated to an extent of 60%. The precipitated solid was filtered off, washed with ethyl acetate and dried under reduced pressure. This gave 454 mg (91% of theory) of the title compound.

¹H NMR (400 MHz, DMSO-d₆): δ=0.71-0.93 (m, 2H), 1.05-1.29 (m, 3H), 1.37 (s, 9H), 1.48-1.58 (m, 1H), 1.67 (m, 3H), 2.06-2.16 (m, 1H), 2.24 (s, 3H), 2.74 (m, 2H), 2.95 (dd, 1H), 3.10 (dd, 1H), 4.64-4.81 (m, 1H), 6.73-6.88 (m, 1H), 7.22-7.33 (m, 3H), 7.39 (d, 2H), 7.80 (d, 3H), 7.85 (s, 1H), 7.99 (d, 2H), 8.18 (d, 1H), 10.43 (s, 1H), 12.90 (br. s., 1H), 15.26 (s, 1H).

LC-MS (Method 1): Rₜ=1.17 min; MS (ESIpos): m/z=799.2 [M+H]⁺

### Example 42A

### Methyl 4′-[(2S)-2-{[(trans-4-{[(tert-butoxycarbonyl)amino]methyl}cyclohexyl)carbonyl]amino}-3-({4-[3-(heptafluoropropyl)-1H-1,2,4-triazol-5-yl]phenyl}amino)-3-oxopropyl]-2-methylbiphenyl-4-carboxylate

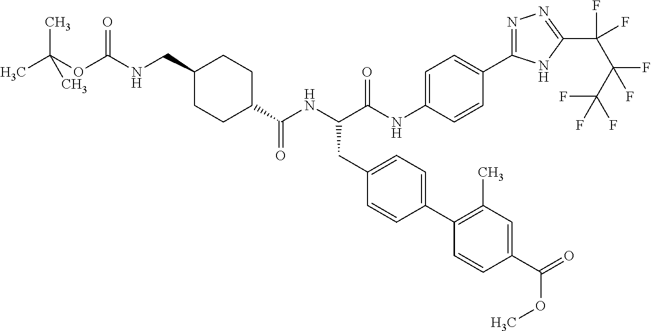

700 mg (0.88 mmol) of 4-bromo-N-alpha-[(trans-4-{[(tert-butoxycarbonyl)amino]methyl}-cyclohexyl)carbonyl]-N-{4-[3-(heptafluoropropyl)-1H-1,2,4-triazol-5-yl]phenyl}-L-phenylalaninamide, 487 mg (1.8 mmol) of methyl 3-methyl-4-(4,4,5,5-tetramethyl-1,3,2-dioxaborolan-2-yl)benzoate and 72 mg (0.088 mmol) of [1,1-bis(diphenylphosphino)ferrocene]dichloropalladium-dichloromethane complex were taken up in 6 ml of 1,2-dimethoxyethane and 2.4 ml of ethanol. After the addition of 0.88 ml of 2N aqueous sodium carbonate solution, the mixture was stirred under reflux for 16 h. The reaction mixture was diluted with dimethylformamide, water and acetonitrile, filtered through a Millipore filter and purified by chromatography via HPLC (acetonitrile/water/0.1% trifluoroacetic acid gradient). The product-containing fractions were combined and concentrated. The residue was recrystallized from methanol and acetonitrile. This gave 532 mg (68% of theory) of the title compound.

¹H NMR (400 MHz, DMSO-d₆): δ=0.71-0.91 (m, 2H), 1.24 (m, 3H), 1.37 (s, 9H), 1.48-1.57 (m, 1H), 1.67 (m, 3H), 2.07-2.17 (m, 1H), 2.24 (s, 3H), 2.74 (m, 2H), 2.94 (dd, 1H), 3.10 (dd, 1H), 3.86 (s, 3H), 4.69-4.79 (m, 1H), 6.75-6.84 (m, 1H), 7.25-7.32 (m, 3H), 7.40 (d, 2H), 7.75-7.84 (m, 3H), 7.88 (s, 1H), 7.99 (d, 2H), 8.19 (d, 1H), 10.43 (s, 1H), 15.30 (s, 1H).

LC-MS (Method 1): Rₜ=1.32 min; MS (ESIpos): m/z=863.4 [M+H]⁺

### Example 43A

### 4′-[(2S)-2-{[(trans-4-{[(tert-Butoxycarbonyl)amino]methyl}cyclohexyl)carbonyl]amino}-3-({4-[3-(heptafluoropropyl)-1H-1,2,4-triazol-5-yl]phenyl}amino)-3-oxopropyl]-2-methylbiphenyl-4-carboxylic acid

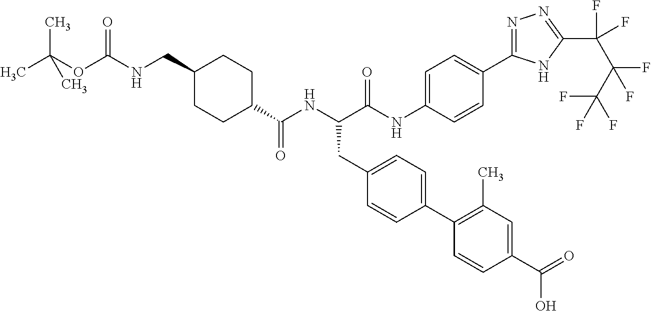

A solution of 527 mg (0.61 mmol) of methyl 4′-[(2S)-2-{[(trans-4-{[(tert-butoxycarbonyl)-amino]methyl}cyclohexyl)carbonyl]amino}-3-({4-[3-(heptafluoropropyl)-1H-1,2,4-triazol-5-yl]-phenyl}amino)-3-oxopropyl]-2-methylbiphenyl-4-carboxylate in 8 ml of tetrahydrofuran/water (3:1) was admixed with 256 mg (6.1 mmol) of lithium hydroxide. The reaction mixture was stirred at RT for 16 h and then at 50° C. for a further 6 h. Subsequently, the mixture was taken up in ethyl acetate and washed with 0.5N hydrochloric acid solution, water and saturated aqueous sodium chloride solution, and the organic phase was dried over sodium sulphate, filtered and concentrated to an extent of 60%. The precipitated solid was filtered off, washed with ethyl acetate and dried under reduced pressure. This gave 505 mg (96% of theory) of the title compound.

¹H NMR (400 MHz, DMSO-d₆): δ=0.83 (d, 2H), 1.06-1.29 (m, 4H), 1.37 (s, 9H), 1.50-1.57 (m, 1H), 1.67 (m, 3H), 2.06-2.17 (m, 1H), 2.24 (s, 3H), 2.75 (m, 2H), 2.95 (br. dd, 1H), 3.11 (br. dd, 1H), 4.69-4.79 (m, 1H), 6.73-6.83 (m, 1H), 7.23-7.31 (m, 3H), 7.39 (d, 2H), 7.80 (m, 3H), 7.85 (s, 1H), 7.99 (d, 2H), 8.18 (d, 1H), 10.43 (s, 1H), 12.90 (br. s., 1H), 15.30 (br. s., 1H).

LC-MS (Method 1): Rₜ=1.21 min; MS (ESIpos): m/z=849.3 [M+H]⁺

### Example 44A

### Methyl 4′-[(2S)-2-{[(trans-4-{[(tert-butoxycarbonyl)amino]methyl}cyclohexyl)carbonyl]amino}-3-{[4-(3-chloro-4H-1,2,4-triazol-5-yl)phenyl]amino}-3-oxopropyl]-2-methylbiphenyl-4-carboxylate

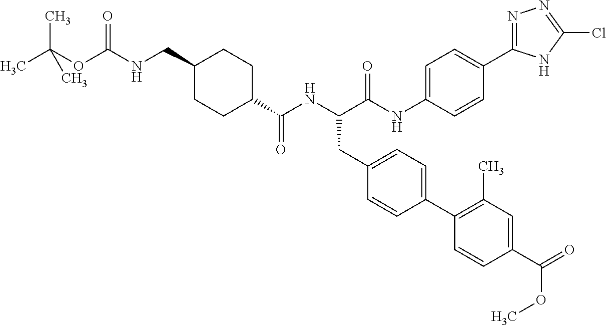

Methyl 3-methyl-4-(4,4,5,5-tetramethyl-1,3,2-dioxaborolan-2-yl)benzoate (1128 mg, 2 mmol), 4-(1H-2-chlorotriazol-5-yl)aniline (596 mg, 3.1 mmol) and N,N-diisopropylethylamine (1.07 ml, 6.1 mmol) were suspended in 12 ml of dimethylformamide and admixed with 2,4,6-tripropyl-1,3,5,2,4,6-trioxatriphosphinane 2,4,6-trioxide (50% in dimethylformamide, 1.79 ml, 3.1 mmol). The mixture was then stirred at RT for 16 h. The reaction mixture was partitioned between water and ethyl acetate, admixed with 1N sodium hydroxide solution and extracted repeatedly with ethyl acetate. The organic phases were washed with saturated aqueous sodium chloride solution and dried over sodium sulphate, and the solvent was removed. This gave 603 mg (41% of theory) of the title compound.

LC-MS (Method 1): Rₜ=1.20 min; MS (ESIpos): m/z=729 [M+H]⁺.

### Example 45A

### 4′-[(2S)-2-{[(trans-4-{[(tert-Butoxycarbonyl)amino]methyl}cyclohexyl)carbonyl]amino}-3-{[4-(3-chloro-4H-1,2,4-triazol-5-yl)phenyl]amino}-3-oxopropyl]-2-methylbiphenyl-4-carboxylic acid

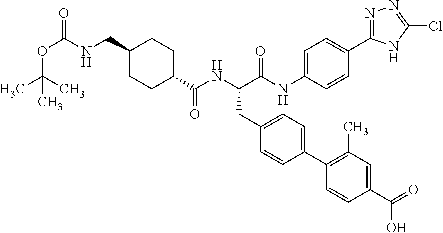 [SMILES: Cc1cc(C(=O)O)ccc1-c1ccc(C[C@H](NC(=O)[C@H]2CC[C@H](CNC(=O)OC(C)(C)C)CC2)C(=O)Nc2ccc(-c3nnc(Cl)[nH]3)cc2)cc1]

A solution of 705 mg (0.97 mmol) of methyl 4′-[(2S)-2-{[(trans-4-{[(tert-butoxycarbonyl)-amino]methyl}cyclohexyl)carbonyl]amino}-3-{[4-(3-chloro-4H-1,2,4-triazol-5-yl)phenyl]amino}-3-oxopropyl]-2-methylbiphenyl-4-carboxylate in 7 ml of tetrahydrofuran was admixed with 4.8 ml (4.8 mmol) of 1M lithium hydroxide solution. The reaction mixture was stirred at RT for 16 h, neutralized with 1N acetic acid solution and separated between ethyl acetate and 10% citric acid solution. The mixture was extracted with ethyl acetate/dioxane, and dried over sodium sulphate and under reduced pressure. The solid formed was suspended with acetonitrile, washed and dried under high vacuum. This gave 507 mg (69% of theory, 93% purity) of the title compound.

LC-MS (Method 1): Rₜ=1.06 min; MS (ESIneg): m/z=714 [M−H]⁻.

### Example 46A

### Methyl 4′-[(2S)-2-{[(trans-4-{[(tert-butoxycarbonyl)amino]methyl}cyclohexyl)carbonyl]amino}-3-oxo-3-{[2-(trifluoromethyl)-1H-benzimidazol-5-yl]amino}propyl]-2-methylbiphenyl-4-carboxylate

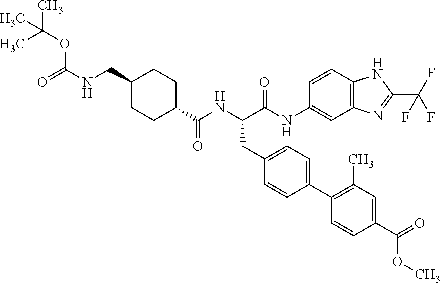 [SMILES: COC(=O)c1ccc(-c2ccc(C[C@H](NC(=O)[C@H]3CC[C@H](CNC(=O)OC(C)(C)C)CC3)C(=O)Nc3ccc4[nH]c(C(F)(F)F)nc4c3)cc2)c(C)c1]

2000 mg (3.6 mmol) of (2S)-2-{[(trans-4-{[(tert-butoxycarbonyl)amino]methyl}-cyclohexyl)-carbonyl]amino}-3-[4′-(methoxycarbonyl)-2′-methylbiphenyl-4-yl]propanoic acid and 946 mg (0.23 mmol) of 2-(trifluoromethyl)-1H-benzimidazol-5-amine in 15 ml of dimethylformamide were admixed with 1.89 ml (10.9 mmol) of N,N-diisopropylethylamine. The reaction mixture was admixed with 3.17 ml (5.4 mmol) of 2,4,6-tripropyl-1,3,5,2,4,6-trioxatriphosphinane 2,4,6-trioxide solution (50% in dimethylformamide) and stirred at RT for 16 h. The reaction mixture was separated between ethyl acetate and water. The mixture was extracted with ethyl acetate, and dried over sodium sulphate and under reduced pressure. The solid formed was suspended with diethyl ether, washed and dried under high vacuum. This gave 1415 mg (51% of theory) of the title compound.

LC-MS (Method 1): Rₜ=1.21 min; MS (ESIneg): m/z=734 [M−H]⁻.

### Example 47A

### 4′-[(2S)-2-{[(trans-4-{[(tert-Butoxycarbonyl)amino]methyl}cyclohexyl)carbonyl]amino}-3-oxo-3-{[2-(trifluoromethyl)-1H-benzimidazol-5-yl]amino}propyl]-2-methylbiphenyl-4-carboxylic acid

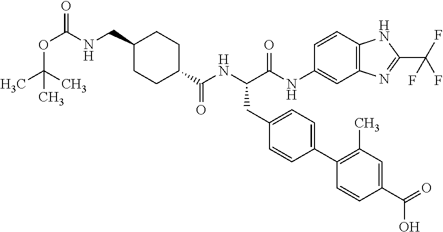 [SMILES: Cc1cc(C(=O)O)ccc1-c1ccc(C[C@H](NC(=O)[C@H]2CC[C@H](CNC(=O)OC(C)(C)C)CC2)C(=O)Nc2ccc3[nH]c(C(F)(F)F)nc3c2)cc1]

A solution of 1350 mg (1.84 mmol) of methyl 4′-[(2S)-2-{[(trans-4-{[(tert-butoxycarbonyl)-amino]methyl}cyclohexyl)carbonyl]amino}-3-oxo-3-{[2-(trifluoromethyl)-1H-benzimidazol-5-yl]amino}propyl]-2-methylbiphenyl-4-carboxylate in 20 ml of tetrahydrofuran was admixed with 9.2 ml (9.2 mmol) of 1M lithium hydroxide solution. The reaction mixture was stirred at RT for 16 h, neutralized with 1N acetic acid solution and separated between ethyl acetate and 10% citric acid solution. The mixture was extracted with ethyl acetate/dioxane, and dried over sodium sulphate and under reduced pressure. The solid formed was suspended with acetonitrile, washed and dried under high vacuum. This gave 829 mg (63% of theory) of the title compound.

LC-MS (Method 1): Rₜ=1.05 min; MS (ESIneg): m/z=720 [M−H]⁻.

### Example 48A

### Methyl 4′-[(2S)-2-{[(trans-4-{[(tert-butoxycarbonyl)amino]methyl}cyclohexyl)carbonyl]amino}-3-oxo-3-{[2-(pentafluoroethyl)-1H-benzimidazol-5-yl]amino}propyl]-2-methylbiphenyl-4-carboxylate

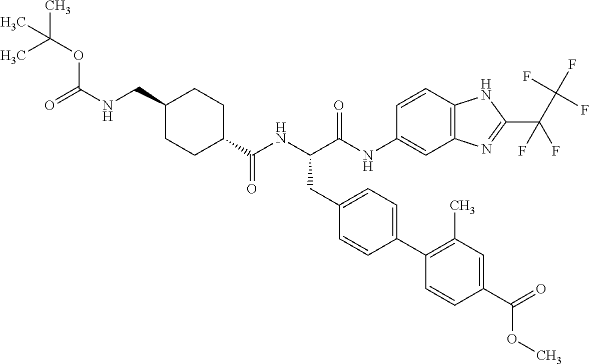 [SMILES: COC(=O)c1ccc(-c2ccc(C[C@H](NC(=O)[C@H]3CC[C@H](CNC(=O)OC(C)(C)C)CC3)C(=O)Nc3ccc4[nH]c(C(F)(F)C(F)(F)F)nc4c3)cc2)c(C)c1]

2500 mg (4.5 mmol) of (2S)-2-{[(trans-4-{[(tert-butoxycarbonyl)amino]methyl}cyclohexyl)-carbonyl]amino}-3-[4′-(methoxycarbonyl)-2′-methylbiphenyl-4-yl]propanoic acid and 1561 mg (5.4 mmol) of 2-(pentafluoroethyl)-1H-benzimidazol-5-amine hydrochloride in 22 ml of dimethylformamide were admixed with 2.36 ml (13.6 mmol) of N,N-diisopropylethylamine. The reaction mixture was admixed with 3.96 ml (6.79 mmol) of 2,4,6-tripropyl-1,3,5,2,4,6-trioxatriphosphinane 2,4,6-trioxide solution (50% in dimethylformamide) and stirred at RT for 16 h. The reaction mixture was admixed with saturated aqueous sodium hydrogencarbonate solution. The solid formed was washed with water and dried under high vacuum. This gave 2145 mg (53% of theory, 88% purity) of the title compound.

LC-MS (Method 1): Rₜ=1.24 min; MS (ESIneg): m/z=784 [M−H]⁻.

### Example 49A

### 4′-[(2S)-2-{[(trans-4-{[(tert-Butoxycarbonyl)amino]methyl}cyclohexyl)carbonyl]amino}-3-oxo-3-{[2-(pentafluoroethyl)-1H-benzimidazol-5-yl]amino}propyl]-2-methylbiphenyl-4-carboxylic acid

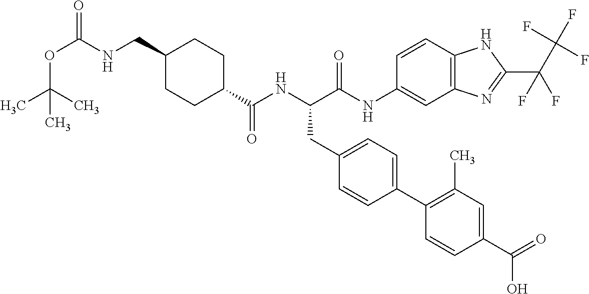 [SMILES: Cc1cc(C(=O)O)ccc1-c1ccc(C[C@H](NC(=O)[C@H]2CC[C@H](CNC(=O)OC(C)(C)C)CC2)C(=O)Nc2ccc3[nH]c(C(F)(F)C(F)(F)F)nc3c2)cc1]

A solution of 1100 mg (1.40 mmol) of methyl 4′-[(2S)-2-{[(trans-4-{[(tert-butoxycarbonyl)-amino]methyl}cyclohexyl)carbonyl]amino}-3-oxo-3-{[2-(pentafluoroethyl)-1H-benzimidazol-5-yl]amino}propyl]-2-methylbiphenyl-4-carboxylate in 10 ml of tetrahydrofuran was admixed with 10 ml (10 mmol) of 1M lithium hydroxide solution. The reaction mixture was stirred at RT for 4 h and separated between ethyl acetate and 10% citric acid solution. The mixture was extracted with ethyl acetate/dioxane, and dried over sodium sulphate and under reduced pressure. The residue was purified by chromatography (silica gel, dichloromethane/methanol 10:1) and the solvent was removed. This gave 699 mg (61% of theory) of the title compound.

LC-MS (Method 1): Rₜ=1.10 min; MS (ESIneg): m/z=770 [M−H]⁻.

### Example 50A

### (2S)-2-{[(trans-4-{[(tert-Butoxycarbonyl)amino]methyl}cyclohexyl)carbonyl]amino}-3-[4′-(methoxycarbonyl)-2′-methylbiphenyl-4-yl]propanoic acid

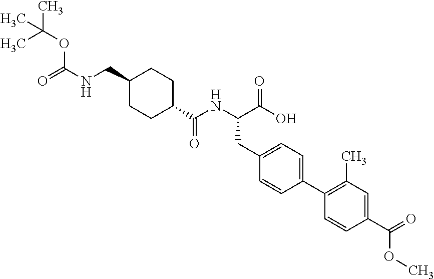 [SMILES: COC(=O)c1ccc(-c2ccc(C[C@H](NC(=O)[C@H]3CC[C@H](CNC(=O)OC(C)(C)C)CC3)C(=O)O)cc2)c(C)c1]

A degassed solution of 4-bromo-N-[(trans-4-{[(tert-butoxycarbonyl)amino]methyl}-cyclohexyl)carbonyl]-L-phenylalanine (9.36 mg, 19.36 mmol), pinacol 2-methyl-4-methoxycarbonylphenylboronate (6.95 g, 25 mmol) and 2N aqueous sodium carbonate solution (29 ml) in dimethylformamide (100 ml) was admixed with 1,1′-bis(diphenylphosphino)ferrocenepalladium(II) chloride (1417 mg, 1.9 mmol) and heated at 120° C. for 30 min. The reaction mixture was filtered through kieselguhr and washed through with ethyl acetate. The filtrate was concentrated and separated between ethyl acetate and 10% citric acid solution. The mixture was extracted with ethyl acetate, and dried over sodium sulphate and under reduced pressure. The solid formed was suspended with acetonitrile, washed and dried under high vacuum. This gave 8.77 g (80% of theory) of the title compound.

¹H NMR (400 MHz, DMSO-d₆): δ=0.70-0.89 (m, 2H), 1.02-1.29 (m, 3H), 1.37 (s, 9H), 1.50 (d, 1H), 1.57-1.71 (m, 3H), 2.04 (br. s., 1H), 2.27 (s, 3H), 2.74 (t, 2H), 2.85-2.95 (m, 1H), 3.14 (dd, 1H), 3.87 (s, 3H), 4.49 (m, 1H), 6.68-6.85 (m, 1H), 7.19-7.36 (m, 5H), 7.82 (d, 1H), 7.88 (s, 1H), 8.04 (d, 1H), 12.66 (br. s, 1H).

LC-MS (Method 1): Rₜ=1.09 min; MS (ESIpos): m/z=553 [M+H]⁺.

### Example 51A

### tert-Butyl 4-[(4-bromo-3-methylbenzoyl)amino]piperidine-1-carboxylate

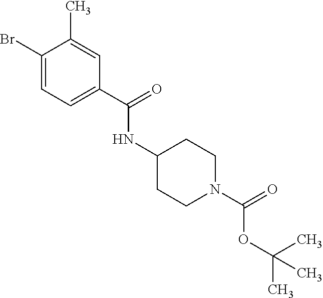 [SMILES: Cc1cc(C(=O)NC2CCN(C(=O)OC(C)(C)C)CC2)ccc1Br]

A solution of 71.5 g (357 mmol) of tert-butyl-4-aminopiperidine-1-carboxylic acid and 76.8 g (357 mmol) of 4-bromo-3-methylbenzoic acid in 1430 ml of ethyl acetate was admixed with 155.46 ml (115 mmol) of N,N-diisopropylethylamine and 340 g (341 mmol) of a 50% 2,4,6-tripropyl-1,3,5,2,4,6-trioxatriphosphinane 2,4,6-trioxide solution in dimethylformamide and stirred at 77° C. for 3 h, then at RT for 16 h. The contents of the flask were admixed with water and the aqueous phase was extracted with ethyl acetate. The combined organic phases were washed with saturated aqueous sodium hydrogencarbonate solution, saturated aqueous ammonium chloride solution and saturated aqueous sodium chloride solution, and dried over sodium sulphate. The solvent was removed and the solid formed was dried under high vacuum. 142 g (70% of theory, 93% purity) of the title compound were obtained.

LC-MS (Method 1): Rₜ=1.16 min; MS (ESIneg): m/z=395 [M−H]⁻.

### Example 52A

### tert-Butyl 4-[({4′-[(2S)-2-{[(trans-4-{[(tert-butoxycarbonyl)amino]methyl}cyclohexyl)carbonyl]-amino}-3-methoxy-3-oxopropyl]-2-methylbiphenyl-4-yl}carbonyl)amino]piperidine-1-carboxylate

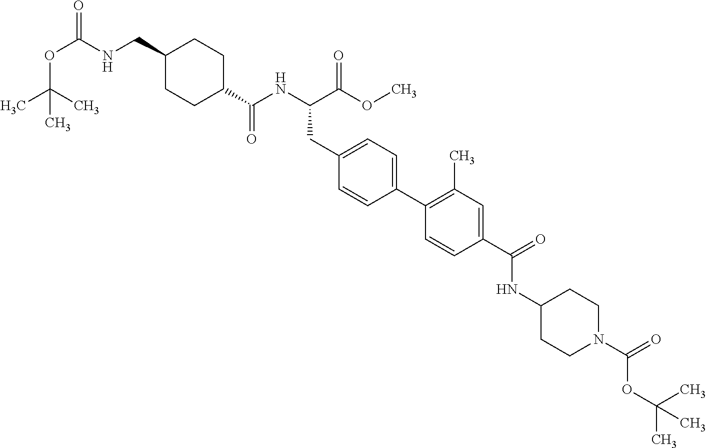

A solution of 2551 mg (5.46 mmol) of tert-butyl 4-[(4-bromo-3-methylbenzoyl)amino]piperidine-1-carboxylate in 20 ml of toluene was admixed with 2.1 mg (8.2 mmol) of bis(pinacolato)diboron and 1607 mg (16.4 mmol) of potassium acetate, and flooded with argon for 10 min. 200 mg (0.27 mmol) of [1,1′-bis(diphenylphosphino)ferrocene]dichloropalladium(II) were added and the mixture was stirred at RT for 16 h. Then 3800 mg (7.64 mmol) of methyl 4-bromo-N-[(trans-4-{[(tert-butoxycarbonyl)-amino]methyl}-cyclohexyl)carbonyl]-L-phenylalaninate and 1157 mg (10.9 mmol) of sodium carbonate were added and the mixture was stirred at RT for 16 h. The contents of the flask were admixed with ethyl acetate and washed with water. The organic phase was dried over sodium sulphate, the solvent was removed and the solid formed was dried under high vacuum. 3000 mg (75% of theory, 72% purity) of the title compound were obtained.

LC-MS (Method 1): Rₜ=1.23 min; MS (ESIpos): m/z=735 [M+H]⁺.

### Example 53A

### (2S)-2-{[(trans-4-{[(tert-Butoxycarbonyl)amino]methyl}cyclohexyl)carbonyl]amino}-3-(4′-{[1-(tert-butoxycarbonyl)piperidin-4-yl]carbamoyl}-2′-methylbiphenyl-4-yl)propanoic acid

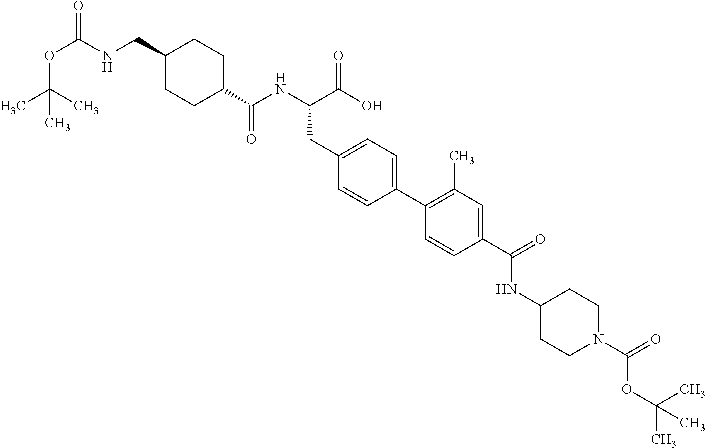

A solution of 500 mg (0.68 mmol) of tert-butyl 4-[({4′-[(2S)-2-{[(trans-4-{[(tert-butoxycarbonyl)amino]methyl}cyclohexyl)carbonyl]-amino}-3-methoxy-3-oxopropyl]-2-methylbiphenyl-4-yl}carbonyl)amino]piperidine-1-carboxylate in 7 ml of tetrahydrofuran was admixed with 3.40 ml (3.40 mmol) of a 1M lithium hydroxide solution in water and stirred at RT for 1 h. The contents of the flask were admixed with 0.20 μl (3.40 mmol) of acetic acid and partitioned between 10% citric acid solution and ethyl acetate. The mixture was extracted twice with ethyl acetate and dried over sodium sulphate. The solvent was removed and the solid formed was dried under high vacuum. 529 mg (quant., 92% purity) of the title compound were obtained.

¹H NMR (400 MHz, DMSO-d₆): δ=0.70-0.89 (m, 2H), 1.04-1.31 (m, 4H), 1.37 (s, 9H), 1.41 (s, 9H), 1.66 (m, 3H), 1.79 (d, 2H), 1.91 (s, 3H), 2.00-2.12 (m, 1H), 2.50 (br. s., 2H) 2.70-2.77 (m, 2H), 2.89 (dd, 3H), 3.11 (d, 1H), 3.83-4.08 (m, 3H), 4.43-4.53 (m, 1H), 6.78 (s, 1H), 7.16-7.36 (m, 5H), 7.65-7.79 (m, 2H), 8.05 (d, 1H), 8.27 (d, 1H), 12.31 (br. s, 1H).

LC-MS (Method 1): Rₜ=1.12 min; MS (ESIneg): m/z=719 [M−H]⁻.

### Example 54A

### tert-Butyl 5-(4-aminophenyl)-3-oxo-2,3-dihydro-1H-pyrazole-1-carboxylate

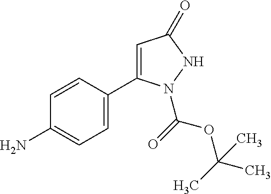 [SMILES: CC(C)(C)OC(=O)n1[nH]c(=O)cc1-c1ccc(N)cc1]

2.50 g (12.19 mmol) of 5-(4-nitrophenyl)-1,2-dihydro-3H-pyrazol-3-one were initially charged in 50 ml of dichloromethane, 1.7 ml (12.19 mmol) of triethylamine and 2.66 g (12.19 mmol) of di-tert-butyl dicarbonate were added and the reaction mixture was stirred at RT for 4 h. Water was added and the mixture was extracted with dichloromethane. The organic phase was dried over sodium sulphate and filtered, and the solvent was removed. The residue was separated by means of column chromatography using silica gel (dichloromethane/methanol 200:1→100:1). The product-containing fractions were concentrated and the residue was dissolved in 100 ml of ethanol. 253 mg of palladium on activated carbon (10%) were added. The suspension was hydrogenated under standard hydrogen pressure at RT for 2 h, then filtered through a filter paper and washed through with a little ethanol. The filtrate was concentrated and dried. This gave 1.99 g (53% of theory, 90% purity) of the title compound over 2 stages.

LC-MS (Method 1): Rₜ=2.06 min; MS (ESIpos): m/z=276 [M+H]⁺.

### Example 55A

### Methyl (2Z)-3-(4-bromo-3-fluorophenyl)-2-[(tert-butoxycarbonyl)amino]acrylate

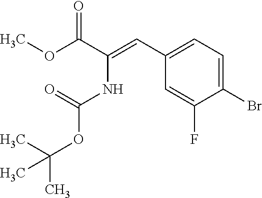 [SMILES: COC(=O)/C(=C/c1ccc(Br)c(F)c1)NC(=O)OC(C)(C)C]

Methyl [(tert-butoxycarbonyl)amino](dimethoxyphosphoryl)acetate (1.46 g, 4.93 mmol) was initially charged under an argon atmosphere in dichloromethane (30 ml), 1,8-diazabicyclo(5.4.0)undec-7-ene (0.82 g, 5.42 mmol) was added and the mixture was stirred at RT for 10 min. A solution of 4-bromo-3-fluorobenzaldehyde (1.00 g, 23 mmol) in dichloromethane (6.5 ml) was added and stirred at RT for 90 min. The reaction mixture was admixed with ethyl acetate and the solution was acidified to about pH 4 with 1N hydrochloric acid solution. The phases were separated and the aqueous phase was extracted twice with ethyl acetate. The combined organic phases were washed with saturated aqueous sodium chloride solution and dried over sodium sulphate, and the solvent was removed. The crude product was applied to silica gel and purified by column chromatography on silica gel (eluent: cyclohexane/ethyl acetate 10:1→5:1), and the solvent was removed. This gave 1.19 g (64% of theory) of the title compound.

¹H NMR (400 MHz, DMSO): δ =1.39 (s, 9H), 3.74 (s, 3H), 7.1 (br. s, 1H), 7.43 (d, 1H), 7.63 (d, 1H), 7.77 (t, 1H), 8.9 (br. s, 1H).

LC-MS (Method 2): Rₜ=2.44 min; MS (ESIneg): m/z=372 [M−H]⁻.

### Example 56A

### Methyl 4-bromo-N-(tert-butoxycarbonyl)-3-fluoro-L-phenylalaninate

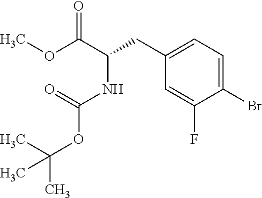 [SMILES: COC(=O)[C@H](Cc1ccc(Br)c(F)c1)NC(=O)OC(C)(C)C]

Methyl (2Z)-3-(4-bromo-3-fluorophenyl)-2-[(tert-butoxycarbonyl)amino]acrylate (1.19 g, 3.17 mmol) was initially charged in ethanol (34 ml), and the mixture was degassed with argon, admixed with (+)-1,2-bis((2S,5S)-2,5-diethylphospholano)benzene(cyclooctadiene)rhodium(I) trifluoromethanesulphonate (49 mg, 0.06 mmol) and stirred at RT under a hydrogen atmosphere (3 bar) for 48 h. The reaction mixture was filtered through kieselguhr, washed with ethanol and concentrated to dryness. This gave 1.11 g (93% of theory) of the title compound.

α-D=−0.014° (23° C., c=0.505 g/100 ml)

¹H NMR (400 MHz, DMSO-d₆): δ=1.22-1.35 (m, 9H), 2.78-2.88 (m, 1H), 2.99-3.07 (m, 1H), 3.63 (s, 3H), 4.16-4.27 (m, 1H), 7.02-7.09 (m, 1H), 7.25-7.38 (m, 2H), 7.61 (t, 1H).

LC-MS (Method 1): Rₜ=1.17 min; MS (ESIpos): m/z=376 [M+H]⁺.

### Example 57A

### Methyl 4-bromo-3-fluoro-L-phenylalaninate hydrochloride

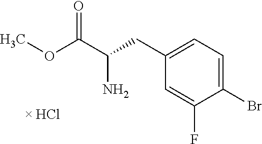

A solution of methyl 4-bromo-N-(tert-butoxycarbonyl)-3-fluoro-L-phenylalaninate (1.05 g, 2.78 mmol) in 1,4-dioxane (20 ml) was admixed with 10.4 ml (41.7 mmol) of 4M hydrogen chloride in 1,4-dioxane and stirred at RT overnight. The precipitate was filtered off, washed with a little acetonitrile and dried under high vacuum. This gave 0.57 g (66% of theory) of the title compound.

LC-MS (Method 1): Rₜ=0.54 min; MS (ESIpos): m/z=276 [M+H—HCl]⁺.

### Example 58A

### tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]piperidine-1-carboxylate

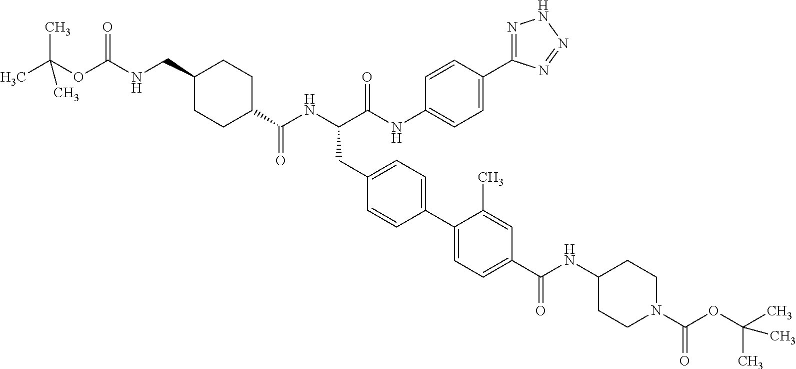

100 mg (0.15 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-methyl-biphenyl-4-carboxylic acid and 35 mg (0.18 mmol) of tert-butyl 4-aminopiperidine-1-carboxylate were dissolved in 5 ml of tetrahydrofuran, 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 82 mg of a mixture of the title compound and the corresponding deprotected amine, which was used directly in the next stage.

LC-MS (Method 1): Rₜ=1.15 min; MS (ESIneg): m/z=862 [M−H]⁻.

### Example 59A

### tert-Butyl [(trans-4-{[(2S)-3-(4′-{[2-(diethylamino)ethyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate

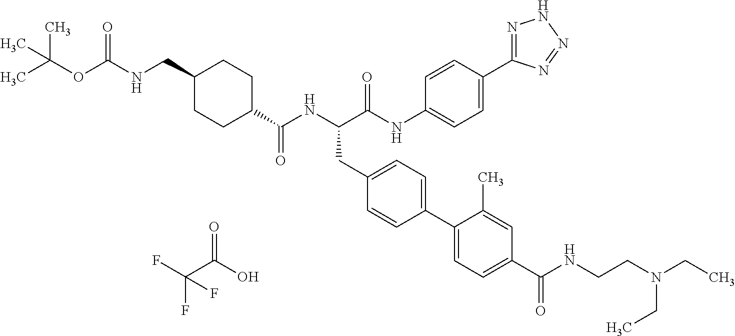

100 mg (0.15 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-methylbiphenyl-4-carboxylic acid and 20 mg (0.18 mmol) of diethylaminoethylamine were dissolved in 5 ml of tetrahydrofuran, 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 64 h. The reaction mixture was separated directly by means of preparative HPLC (eluent: acetonitrile/water with 0.1% TFA (gradient)). This gave 73 mg of a mixture of the title compound and the corresponding deprotected amine, which was used directly in the next stage.

LC-MS (Method 1): Rₜ=0.82 min; MS (ESIneg): m/z=778 [M−H-TFA]⁻.

### Example 60A

### tert-Butyl [(trans-4-{[(2S)-3-[4′-{(2-[(tert-butoxycarbonyl)amino]ethyl}carbamoyl)-2′-methylbiphenyl-4-yl]-1-(1H-indazol-6-ylamino)-1-oxopropan-2-yl]carbamoyl}cyclohexyl)methyl]carbamate trifluoroacetate

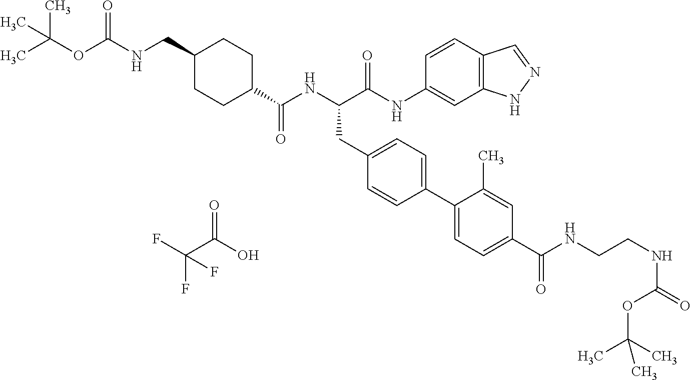

100 mg (0.15 mmol) of 4′-[(2S)-2-{[(trans-4-{[(tert-butoxycarbonyl)amino]methyl}-cyclohexyl)-carbonyl]amino}-3-(1H-indazol-6-ylamino)-3-oxopropyl]-2-methylbiphenyl-4-carboxylic acid and 29 mg (0.18 mmol) of tert-butyl (2-aminoethyl)carbamate were dissolved in 5 ml of tetrahydrofuran, 70 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 79 mg of a mixture of the title compound and the corresponding deprotected amine, which was used directly in the next stage.

LC-MS (Method 1): Rₜ=1.08 min; MS (ESIpos): m/z=796 [M+H-TFA]⁺.

### Example 61A

### tert-Butyl 4-[({4′-[(25)-2-{[(trans-4-{[(tert-butoxycarbonyl)amino]methyl}cyclohexyl)carbonyl]-amino}-3-(1H-indazol-6-ylamino)-3-oxopropyl]-2-methylbiphenyl-4-yl}carbonyl)amino]piperidine-1-carboxylate trifluoroacetate

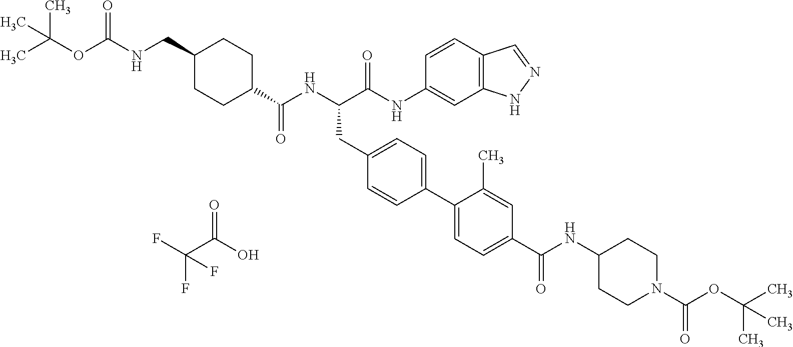

100 mg (0.15 mmol) of 4′-[(2S)-2-{[(trans-4-{[(tert-butoxycarbonyl)amino]methyl}-cyclohexyl)-carbonyl]amino}-3-(1H-indazol-6-ylamino)-3-oxopropyl]-2-methylbiphenyl-4-carboxylic acid and 37 mg (0.18 mmol) of tert-butyl 4-aminopiperidine-1-carboxylate were dissolved in 5 ml of tetrahydrofuran, 70 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 87 mg of a mixture of the title compound and the corresponding deprotected amine, which was used directly in the next stage.

LC-MS (Method 1): Rₜ=1.16 min; MS (ESIpos): m/z=836 [M+H-TFA]⁺.

### Example 62A

### tert-Butyl 4-({4′-[(2S)-2-{[(trans-4-{[(tert-butoxycarbonyl)amino]methyl}cyclohexyl)carbonyl]-amino}-3-(1H-indazol-6-ylamino)-3-oxopropyl]-2-methylbiphenyl-4-yl}carbonyl)piperazine-1-carboxylate trifluoroacetate

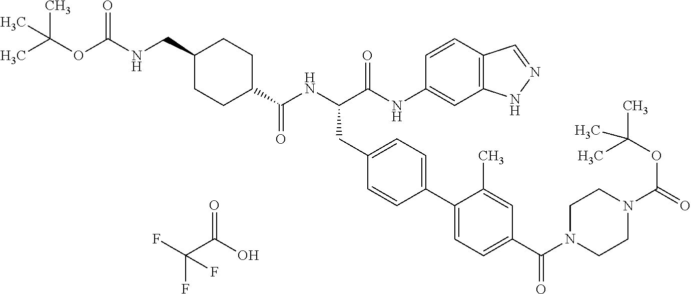

100 mg (0.15 mmol) of 4′-[(2S)-2-{[(trans-4-{[(tert-butoxycarbonyl)amino]methyl}-cyclohexyl)-carbonyl]amino}-3-(1H-indazol-6-ylamino)-3-oxopropyl]-2-methylbiphenyl-4-carboxylic acid and 34 mg (0.18 mmol) of tert-butyl piperazine-1-carboxylate were dissolved in 5 ml of tetrahydrofuran, 70 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 101 mg of a mixture of the title compound and the corresponding deprotected amine, which was used directly in the next stage.

LC-MS (Method 1): Rₜ=1.15 min; MS (ESIpos): m/z=822 [M+H-TFA]⁺.

### Example 63A

### tert-Butyl [(trans-4-{[(2S)-3-(4′-{[2-(diethylamino)ethyl]carbamoyl}-2′-methylbiphenyl-4-yl)-1-(1H-indazol-6-ylamino)-1-oxopropan-2-yl]carbamoyl}cyclohexyl)methyl]carbamate trifluoroacetate

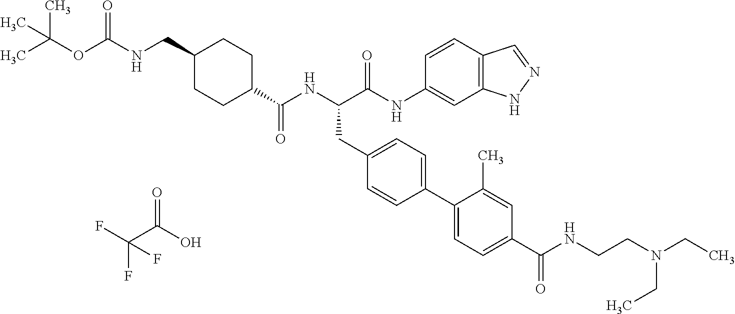

100 mg (0.15 mmol) of 4′-[(2S)-2-{[(trans-4-{[(tert-butoxycarbonyl)amino]methyl}-cyclohexyl)-carbonyl]amino}-3-(1H-indazol-6-ylamino)-3-oxopropyl]-2-methylbiphenyl-4-carboxylic acid and 21 mg (0.18 mmol) of diethylaminoethylamine were dissolved in 5 ml of tetrahydrofuran, 70 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 60 mg of a mixture of the title compound and the corresponding deprotected amine, which was used directly in the next stage.

LC-MS (Method 1): Rₜ=0.80 min; MS (ESIpos): m/z=752 [M+H-TFA]⁺.

### Example 64A

### tert-Butyl [(trans-4-{[(2S)-3-[4′-(isopropylcarbamoyl)-2′-methylbiphenyl-4-yl]-1-oxo-1-{[4-(2H-tetrazol-5-yl)phenyl]amino}propan-2-yl]carbamoyl}cyclohexyl)methyl]carbamate

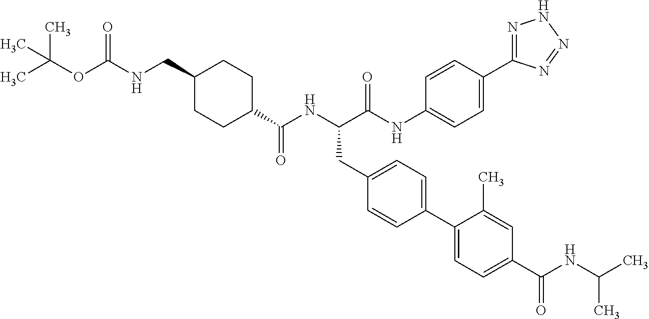

100 mg (0.15 mmol) of 4′-[(2S)-2-{[(trans-4-{[(tert-butoxycarbonyl)amino]methyl}-cyclohexyl)-carbonyl]amino}-3-(2H-tetrazol-5-yl)phenyl]amino}propyl]-2-methylbiphenyl-4-carboxylic acid and 10 mg (0.18 mmol) of isopropylamine were dissolved in 5 ml of tetrahydrofuran, 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 49 mg of a mixture of the title compound and the corresponding deprotected amine, which was used directly in the next stage.

LC-MS (Method 1): Rₜ=1.06 min; MS (ESIneg): m/z=721 [M−H]⁻.

### Example 65A

### tert-Butyl (3R)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]pyrrolidine-1-carboxylate

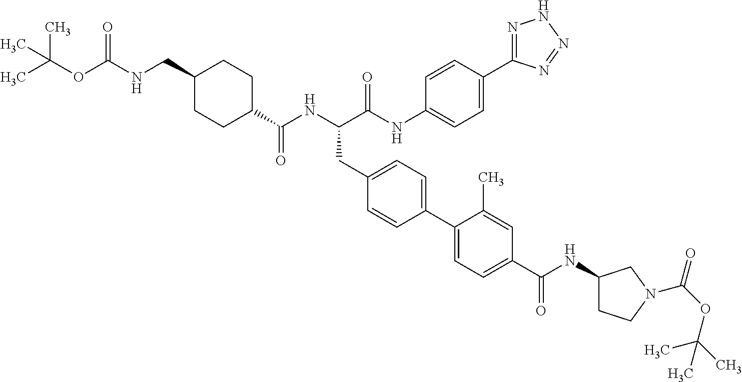

100 mg (0.15 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-methylbiphenyl-4-carboxylic acid and 33 mg (0.18 mmol) of tert-butyl (3R)-pyrrolidin-3-ylcarbamate were dissolved in 5 ml of tetrahydrofuran, 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 61 mg of a mixture of the title compound and the corresponding deprotected amine, which was used directly in the next stage.

LC-MS (Method 1): Rₜ=1.12 min; MS (ESIneg): m/z=848 [M−H]⁻.

### Example 66A

### tert-Butyl (3S)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]pyrrolidine-1-carboxylate

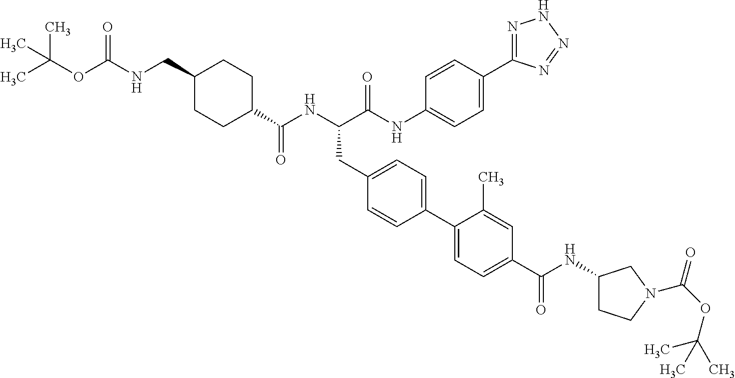

100 mg (0.15 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-methylbiphenyl-4-carboxylic acid and 33 mg (0.18 mmol) of tert-butyl (3S)-pyrrolidin-3-ylcarbamate were dissolved in 5 ml of tetrahydrofuran, 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 59 mg of a mixture of the title compound and the corresponding deprotected amine, which was used directly in the next stage.

LC-MS (Method 1): Rₜ=1.12 min; MS (ESIneg): m/z=848 [M−H]⁻.

### Example 67A

### tert-Butyl [(trans-4-{[(2S)-3-(2′-chloro-4′-{[2-(diethylamino)ethyl]carbamoyl}biphenyl-4-yl)-1-oxo-1-{[4-(2H-tetrazol-5-yl)phenyl]amino}propan-2-yl]carbamoyl}cyclohexyl)methyl]carbamate trifluoroacetate

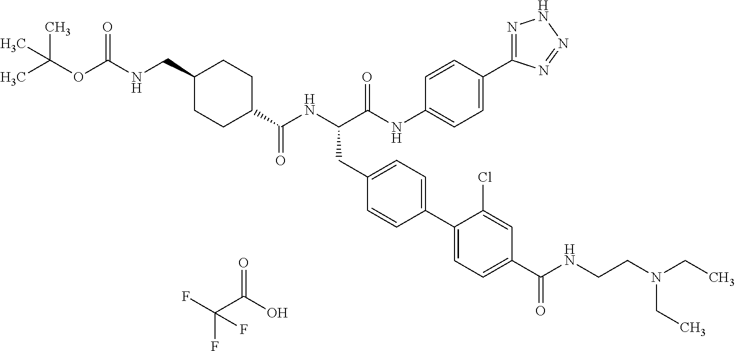

100 mg (0.14 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-chlorobiphenyl-4-carboxylic acid and 20 mg (0.17 mmol) of diethylaminoethylamine were dissolved in 5 ml of tetrahydrofuran, 65 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine were added and the solution was stirred at RT for 64 h. The reaction mixture was separated directly by means of preparative HPLC (eluent: acetonitrile/water with 0.1% TFA (gradient)). This gave 75 mg of a mixture of the title compound and the corresponding deprotected amine, which was used directly in the next stage.

LC-MS (Method 1): Rₜ=0.91 min; MS (ESIneg): m/z=798 [M−H-TFA]⁻.

### Example 68A

### tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-chlorobiphenyl-4-yl}carbonyl)amino]piperidine-1-carboxylate

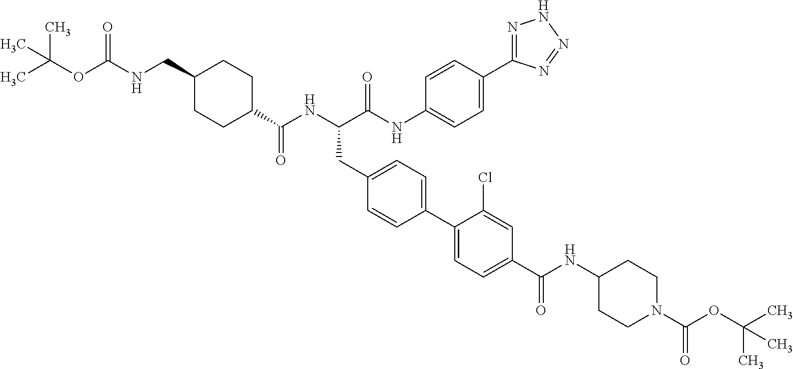

100 mg (0.14 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-chlorobiphenyl-4-carboxylic acid and 34 mg (0.17 mmol) of tert-butyl 4-aminopiperidine-1-carboxylate were dissolved in 5 ml of tetrahydrofuran, 65 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 68 mg of a mixture of the title compound and the corresponding deprotected amine, which was used directly in the next stage.

LC-MS (Method 1): Rₜ=1.17 min; MS (ESIneg): m/z=882 [M−H]⁻.

### Example 69A

### tert-Butyl (3R)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-chlorobiphenyl-4-yl}carbonyl)amino]pyrrolidine-1-carboxylate

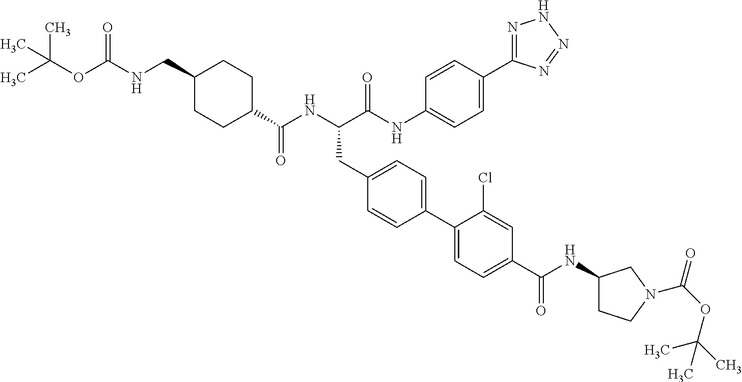

100 mg (0.14 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-chlorobiphenyl-4-carboxylic acid and 32 mg (0.17 mmol) of tert-butyl (3R)-pyrrolidin-3-ylcarbamate were dissolved in 5 ml of tetrahydrofuran, 65 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 90 mg of a mixture of the title compound and the corresponding deprotected amine, which was used directly in the next stage.

LC-MS (Method 1): Rₜ=1.15 min; MS (ESIneg): m/z=868 [M−H]⁻.

### Example 70A

### tert-Butyl (3S)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-chlorobiphenyl-4-yl}carbonyl)amino]pyrrolidine-1-carboxylate

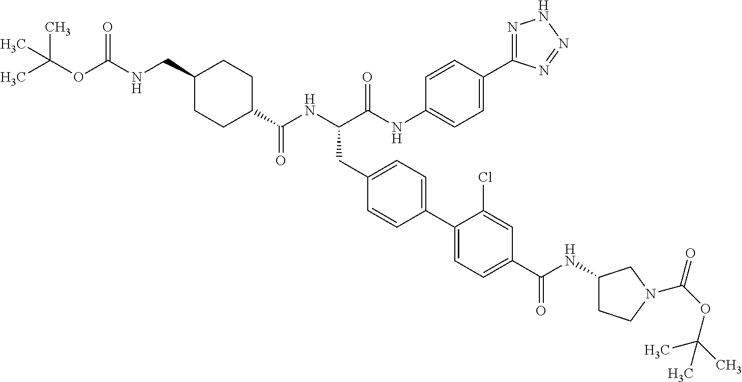

100 mg (0.14 mmol) of 4′-[(2S)-2-{[(trans-4-{[(tert-butoxycarbonyl)amino]methyl}-cyclohexyl)-carbonyl]amino}-3-oxo-3-{[4-(2H-tetrazol-5-yl)phenyl]amino}propyl]-2-chlorobiphenyl-4-carboxylic acid and 32 mg (0.17 mmol) of tert-butyl (3S)-pyrrolidin-3-ylcarbamate were dissolved in 5 ml of tetrahydrofuran, 65 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 88 mg of a mixture of the title compound and the corresponding deprotected amine, which was used directly in the next stage.

LC-MS (Method 1): Rₜ=1.15 min; MS (ESIneg): m/z=868 [M−H]⁻.

### Example 71A

### tert-Butyl [(trans-4-{[(2S)-3-(4′-carbamoyl-2′-methylbiphenyl-4-yl)-1-oxo-1-{[4-(2H-tetrazol-5-yl)phenyl]amino}propan-2-yl]carbamoyl}cyclohexyl)methyl]carbamate

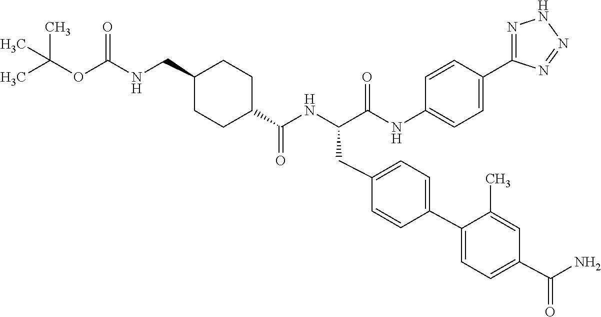

A solution of 80 mg (0.10 mmol, 83% purity) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid in 5 ml of tetrahydrofuran was admixed with 44 mg (0.12 mmol) of N-[(dimethylamino)(3H-[1,2,3]triazolo[4,5-b]pyridin-3-yloxy)methylidene]-N-methylmethanaminium hexafluorophosphate, 0.02 ml (0.12 mmol) of N,N-diisopropylethylamine and 0.24 ml (0.5 mmol) of a 2M ammonia solution in methanol, and stirred at RT for 24 h. After adding another 19 mg (0.05 mmol) of N-[(dimethylamino)(3H-[1,2,3]triazolo[4,5-b]pyridin-3-yloxy)methylidene]-N-methylmethanaminium hexafluorophosphate and 0.15 ml (0.29 mmol) of a 2M ammonia solution in methanol, the mixture was stirred at RT for a further 2 d. The reaction mixture was separated directly by means of preparative HPLC (eluent: acetonitrile/water with 0.1% TFA (gradient)). This gave 51 mg of a mixture of the title compound and the corresponding deprotected amine, which was used directly in the next stage.

LC-MS (Method 1): Rₜ=0.93 min; MS (ESIneg): m/z=679 [M−H]⁻.

### Example 72A

### tert-Butyl [(trans-4-{[(2S)-3-[4′-({2-[(tert-butoxycarbonyl)amino]ethyl}carbamoyl)-2′-methylbiphenyl-4-yl]-1-oxo-1-{[4-(2H-tetrazol-5-yl)phenyl]amino}propan-2-yl]carbamoyl}-cyclohexyl)methyl]carbamate

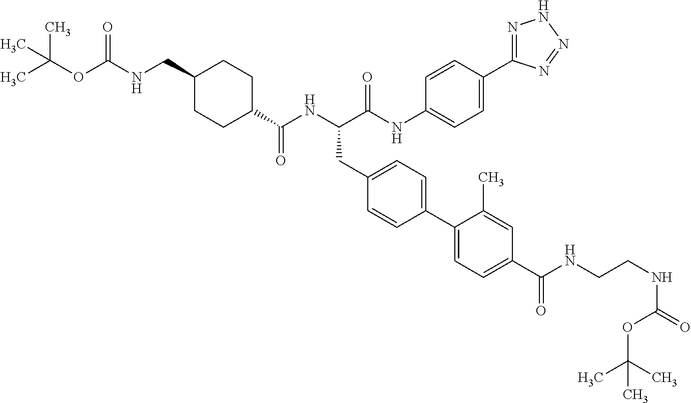

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 28 mg (0.18 mmol) of tert-butyl (2-aminoethyl)carbamate in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine, and stirred at RT for 24 h. The reaction mixture was separated directly by means of preparative HPLC (acetonitrile/water gradient). This gave 96 mg of a mixture of the title compound and the corresponding deprotected amine, which was used directly in the next stage.

LC-MS (Method 1): Rₜ=1.06 min; MS (ESIneg): m/z=822 [M−H]⁻.

### Example 73A

### tert-Butyl 4-({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methylbiphenyl-4-yl}carbonyl)piperazine-1-carboxylate

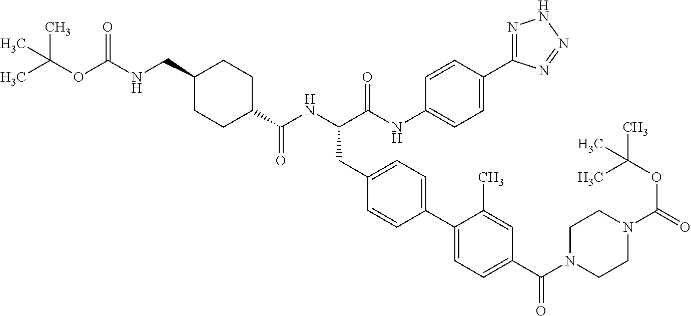

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 33 mg (0.18 mmol) of tert-butyl piperazine-1-carboxylate in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine, and stirred at RT for 16 h. The reaction mixture was separated directly by means of preparative HPLC (eluent: acetonitrile/water with 0.1% TFA (gradient)). This gave 95 mg of a mixture of the title compound and the corresponding deprotected amine, which was used directly in the next stage.

LC-MS (Method 1): Rₜ=1.14 min; MS (ESIneg): m/z=848 [M−H]⁻.

### Example 74A

### tert-Butyl [(trans-4-{[(2S)-3-{4′-[(2-tert-butoxyethyl)carbamoyl]-2′-methylbiphenyl-4-yl}-1-(1H-indazol-6-ylamino)-1-oxopropan-2-yl]carbamoyl}cyclohexyl)methyl]carbamate trifluoroacetate

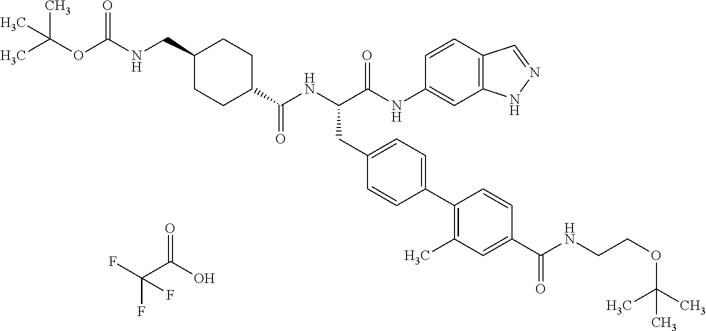

A solution of 100 mg (0.15 mmol) of 4′-[(2S)-2-{[(trans-4-{[(tert-butoxycarbonyl)amino]methyl}-cyclohexyl)-carbonyl]amino}-3-(1H-indazol-6-ylamino)-3-oxopropyl]-2-methylbiphenyl-4-carboxylic acid and 28 mg (0.18 mmol) of tert-butoxyethanamine in 5 ml of tetrahydrofuran was admixed with 70 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 102 mg of a mixture of the title compound and the corresponding deprotected amine, which was used directly in the next stage.

LC-MS (Method 1): Rₜ=1.11 min; MS (ESIpos): m/z=753 [M+H-TFA]⁺.

### Example 75A

### tert-Butyl [(trans-4-{[(2S)-3-{4′-[(2-tert-butoxyethyl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate

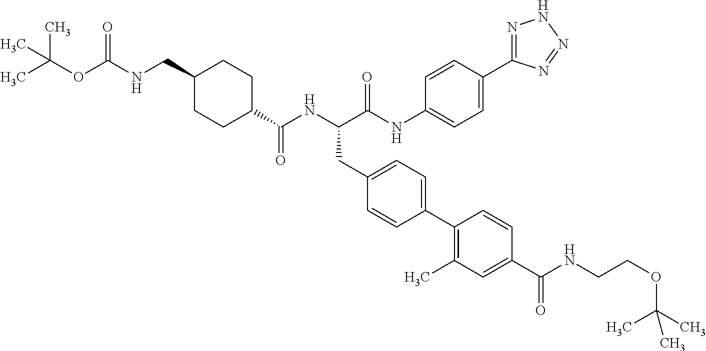

A solution of 125 mg (0.15 mmol, 79% purity) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 27 mg (0.17 mmol) of tert-butoxyethanamine in 5 ml of tetrahydrofuran was admixed with 66 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine, and stirred at RT for 16 h. The reaction mixture was separated directly by means of preparative HPLC (eluent: acetonitrile/water with 0.1% TFA (gradient)). This gave 85 mg (49% of theory, 75% purity) of the title compound.

LC-MS (Method 1): Rₜ=1.10 min; MS (ESIneg): m/z=780 [M−H]⁻.

### Example 76A

### tert-Butyl [(trans-4-{[(2S)-3-[2′-methyl-4′-(methylcarbamoyl)biphenyl-4-yl]-1-oxo-1-{[4-(2H-tetrazol-5-yl)phenyl]amino}propan-2-yl]carbamoyl}cyclohexyl)methyl]carbamate

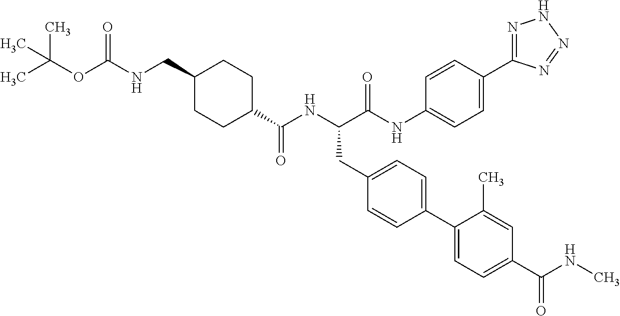

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 0.09 ml (0.18 mmol) of a 2M methylamine solution (in tetrahydrofuran) in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine, and stirred at RT for 16 h. After again adding a further 56 mg (0.15 mmol) of N-[(dimethylamino)(3H-[1,2,3]triazolo[4,5-b]pyridin-3-yloxy)methylidene]-N-methylmethanaminium hexafluorophosphate and 0.20 ml (1.17 mmol) of a 2M methylamine solution (in tetrahydrofuran), the mixture was stirred at RT for a further 48 h. The reaction mixture was separated directly by means of preparative HPLC (eluent: acetonitrile/water with 0.1% TFA (gradient)). This gave 74 mg (19% of theory, 30% purity) of the title compound.

LC-MS (Method 1): Rₜ=0.97 min; MS (ESIpos): m/z=695 [M+H]⁺.

### Example 77A

### tert-Butyl [(trans-4-{[(2S)-3-[2′-methyl-4′-(pyrrolidin-1-ylcarbonyl)biphenyl-4-yl]-1-oxo-1-{[4-(2H-tetrazol-5-yl)phenyl]amino}propan-2-yl]carbamoyl}cyclohexyl)methyl]carbamate

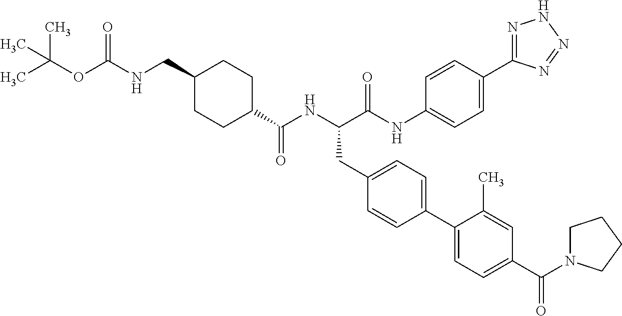

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 0.02 ml (0.18 mmol) of pyrrolidine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine, and stirred at RT for 16 h. After adding another 56 mg (0.15 mmol) of N-[(dimethylamino)(3H-[1,2,3]triazolo[4,5-b]pyridin-3-yloxy)methylidene]-N-methylmethanaminium hexafluorophosphate and 0.06 ml (0.73 mmol) of pyrrolidine, the mixture was stirred at RT for an additional 24 h. The precipitate formed was brought into solution by adding 1 ml of dimethylformamide and the reaction solution was stirred at RT for a further 24 h. The reaction solution was separated directly by means of preparative HPLC (acetonitrile/water gradient). This gave 44 mg of a mixture of the title compound and the corresponding deprotected amine, which was used directly in the next stage.

LC-MS (Method 1): Rₜ=1.05 min; MS (ESIneg): m/z=733 [M−H]⁻.

### Example 78A

### tert-Butyl {[trans-4-({(2S)-1-(1H-indazol-6-ylamino)-3-[4′-(isopropylcarbamoyl)-2′-methylbiphenyl-4-yl]-1-oxopropan-2-yl}carbamoyl)cyclohexyl]methyl}carbamate trifluoroacetate

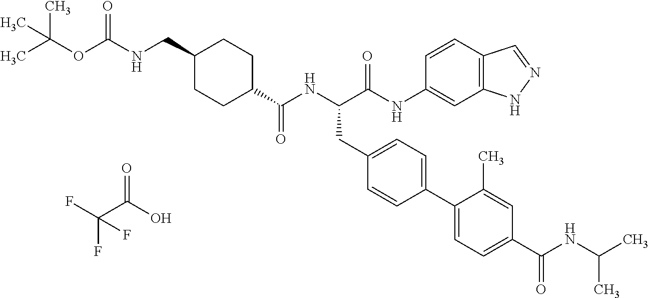

100 mg (0.15 mmol) of 4′-[(2S)-2-{[(trans-4-{[(tert-butoxycarbonyl)amino]methyl}-cyclohexyl)-carbonyl]amino}-3-(1H-indazol-6-ylamino)-3-oxopropyl]-2-methylbiphenyl-4-carboxylic acid and 0.02 ml (0.18 mmol) of isopropylamine were dissolved in 5 ml of tetrahydrofuran, 70 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine were added and the solution was stirred at RT for 16 h. The reaction mixture was separated directly by means of preparative HPLC (eluent: acetonitrile/water with 0.1% TFA (gradient)). This gave 95 mg of a mixture of the title compound and the corresponding deprotected amine, which was used directly in the next stage.

LC-MS (Method 1): Rₜ=1.08 min; MS (ESIneg): m/z=693 [M−H-TFA]⁻.

### Example 79A

### tert-Butyl 4-{[(4′-{(2S)-2-{[(trans-4-{[(tert-butoxycarbonyl)amino]methyl}cyclohexyl)-carbonyl]amino}-3-oxo-3-[(3-oxo-2,3-dihydro-1H-indazol-6-yl)amino]propyl}-2-methylbiphenyl-4-yl)carbonyl]amino}piperidine-1-carboxylate

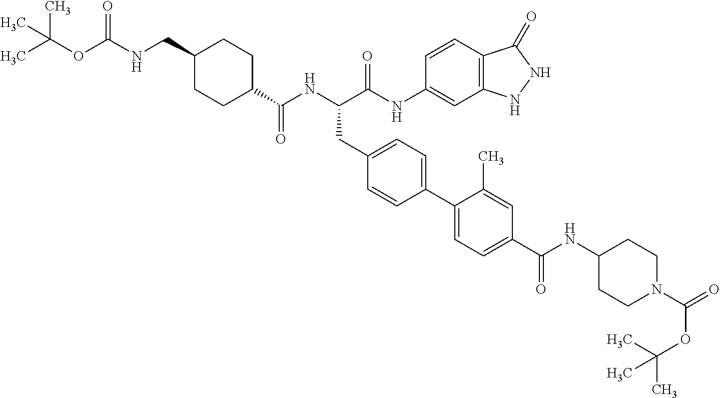

A solution of 150 mg (0.19 mmol) of 4′-{(2S)-2-{[(trans-4-{[(tert-butoxycarbonyl)-amino]methyl}cyclohexyl)carbonyl]amino}-3-oxo-3-[(3-oxo-2,3-dihydro-1H-indazol-6-yl)-amino]propyl}-2-methylbiphenyl-4-carboxylic acid and 46 mg (0.23 mmol) of tert-butyl 4-aminopiperidine-1-carboxylate in 6 ml of tetrahydrofuran was admixed with 87 mg (0.23 mmol) of N-[(dimethylamino)(3H-[1,2,3]triazolo[4,5-b]pyridin-3-yloxy)methylidene]-N-methylmethanaminium hexafluorophosphate and 0.04 ml (0.23 mmol) of N,N-diisopropylethylamine and stirred at RT for 16 h. The reaction mixture was separated directly by means of preparative HPLC (eluent: acetonitrile/water with 0.1% TFA (gradient)). This gave 110 mg of a mixture of the title compound and the corresponding deprotected amine, which was used directly in the next stage.

LC-MS (Method 1): Rₜ=1.08 min; MS (ESIneg): m/z=850 [M−H]⁻.

### Example 80A

### tert-Butyl {[trans-4-({(2S)-3-(4′-{[2-(diethylamino)ethyl]carbamoyl}-2′-methylbiphenyl-4-yl)-1-oxo-1-[(3-oxo-2,3-dihydro-1H-indazol-6-yl)amino]propan-2-yl}carbamoyl)cyclohexyl]methyl}-carbamate trifluoroacetate

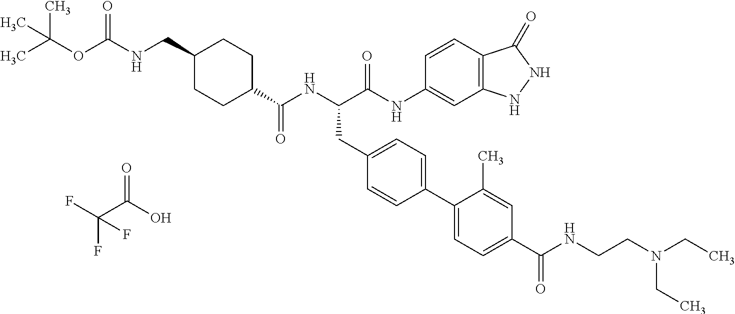

A solution of 100 mg (0.13 mmol, 85% purity) of 4′-{(2S)-2-{[(trans-4-{[(tert-butoxycarbonyl)amino]methyl}cyclohexyl)carbonyl]amino}-3-oxo-3-[(3-oxo-2,3-dihydro-1H-indazol-6-yl)amino]propyl}-2-methylbiphenyl-4-carboxylic acid and 0.02 ml (0.15 mmol) of diethylaminoethylamine in 5 ml of tetrahydrofuran was admixed with 58 mg (0.15 mmol) of N-[(dimethylamino)(3H-[1,2,3]triazolo[4,5-b]pyridin-3-yloxy)methylidene]-N-methylmethanaminium hexafluorophosphate and 0.03 ml (0.15 mmol) of N,N-diisopropylethylamine and stirred at RT for 16 h. The reaction mixture was separated directly by means of preparative HPLC (eluent: acetonitrile/water with 0.1% TFA (gradient)). This gave 60 mg of a mixture of the title compound and the corresponding deprotected amine, which was used directly in the next stage.

LC-MS (Method 1): Rₜ=0.79 min; MS (ESIneg): m/z=766 [M−H-TFA]⁻.

### Example 81A

### tert-Butyl {[trans-4-({(2S)-3-(4′-{[3-(diethylamino)propyl]carbamoyl}-2′-methylbiphenyl-4-yl)-1-oxo-1-[(3-oxo-2,3-dihydro-1H-indazol-6-yl)amino]propan-2-yl}carbamoyl)cyclohexyl]methyl}-carbamate trifluoroacetate

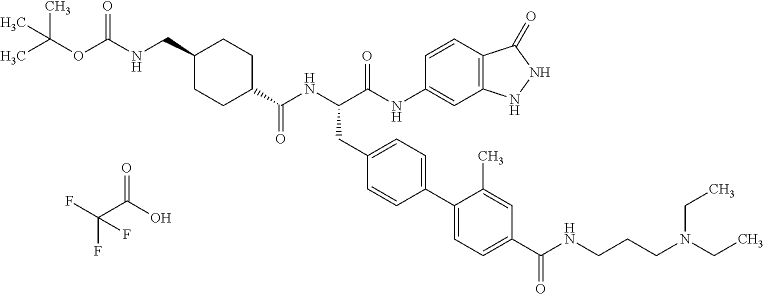

A solution of 100 mg (0.13 mmol) of 4′-{(2S)-2-{[(trans-4-{[(tert-butoxycarbonyl)-amino]methyl}cyclohexyl)carbonyl]amino}-3-oxo-3-[(3-oxo-2,3-dihydro-1H-indazol-6-yl)-amino]propyl}-2-methylbiphenyl-4-carboxylic acid and 20 mg (0.15 mmol) of diethylaminopropylamine in 4 ml of tetrahydrofuran was admixed with 58 mg (0.15 mmol) of N-[(dimethylamino)(3H-[1,2,3]triazolo[4,5-b]pyridin-3-yloxy)methylidene]-N-methylmethanaminium hexafluorophosphate and 0.03 ml (0.15 mmol) of N,N-diisopropylethylamine and stirred at RT for 16 h. The reaction mixture was separated directly by means of preparative HPLC (eluent: acetonitrile/water with 0.1% TFA (gradient)). This gave 44 mg of a mixture of the title compound and the corresponding deprotected amine, which was used directly in the next stage.

LC-MS (Method 1): Rₜ=0.81 min; MS (ESIneg): m/z=780 [M−H-TFA]⁻.

### Example 82A

### tert-Butyl (3S)-3-{[(4′-{(2S)-2-{[(trans-4-{[(tert-butoxycarbonyl)amino]methyl}cyclohexyl)-carbonyl]amino}-3-oxo-3-[(3-oxo-2,3-dihydro-1H-indazol-6-yl)amino]propyl}-2-methylbiphenyl-4-yl)carbonyl]amino}pyrrolidine-1-carboxylate

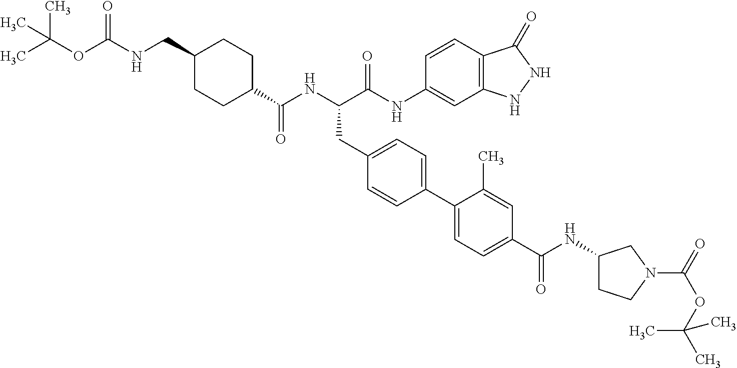

A solution of 100 mg (0.13 mmol, 85% purity) of 4′-{(2S)-2-{[(trans-4-{[(tert-butoxycarbonyl)-amino]methyl}cyclohexyl)carbonyl]amino}-3-oxo-3-[(3-oxo-2,3-dihydro-1H-indazol-6-yl)-amino]propyl}-2-methylbiphenyl-4-carboxylic acid and 28 mg (0.15 mmol) of (S)—N-(tert-butoxycarbonyl)-3-aminopyrrolidine in 4 ml of tetrahydrofuran was admixed with 58 mg (0.15 mmol) of N-[(dimethylamino)(3H-[1,2,3]triazolo[4,5-b]pyridin-3-yloxy)methylidene]-N-methylmethanaminium hexafluorophosphate and 0.03 ml (0.15 mmol) of N,N-diisopropylethylamine and stirred at RT for 16 h. The reaction mixture was separated directly by means of preparative HPLC (eluent: acetonitrile/water with 0.1% TFA (gradient)). This gave 51 mg of a mixture of the title compound and the corresponding deprotected amine, which was used directly in the next stage.

LC-MS (Method 1): Rₜ=1.06 min; MS (ESIneg): m/z=836 [M−H]⁻.

### Example 83A

### tert-Butyl [(trans-4-{[(2S)-3-[2′-chloro-4′-(2,5,8,11-tetraoxatridecan-13-ylcarbamoyl)biphenyl-4-yl]-1-oxo-1-{[4-(2H-tetrazol-5-yl)phenyl]amino}propan-2-yl]carbamoyl}cyclohexyl)methyl]-carbamate

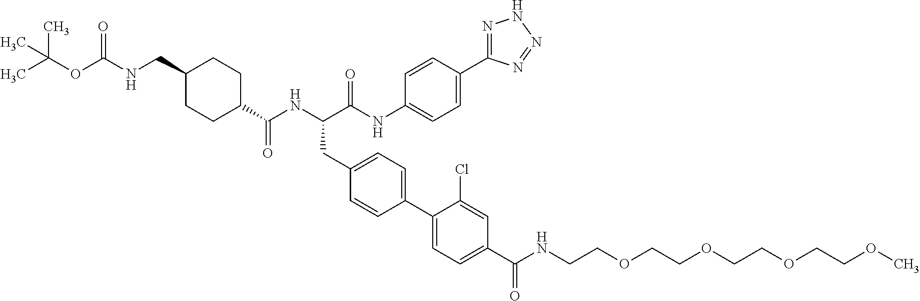

A solution of 100 mg (0.14 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-chlorobiphenyl-4-carboxylic acid and 35 mg (0.17 mmol) of 2,5,8,11-tetraoxatridecan-13-amine in 5 ml of tetrahydrofuran was admixed with 65 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine and stirred at RT for 16 h. The reaction mixture was separated directly by means of preparative HPLC (eluent: acetonitrile/water with 0.1% TFA (gradient)). This gave 80 mg of a mixture of the title compound and the corresponding deprotected amine, which was used directly in the next stage.

LC-MS (Method 1): Rₜ=1.02 min; MS (ESIneg): m/z=889 [M−H]⁻.

### Example 84A

### tert-Butyl [(trans-4-{[(2S)-3-[2′-methyl-4′-(2,5,8,11-tetraoxatridecan-13-ylcarbamoyl)biphenyl-4-yl]-1-oxo-1-{[4-(2H-tetrazol-5-yl)phenyl]amino}propan-2-yl]carbamoyl}cyclohexyl)-methyl]carbamate

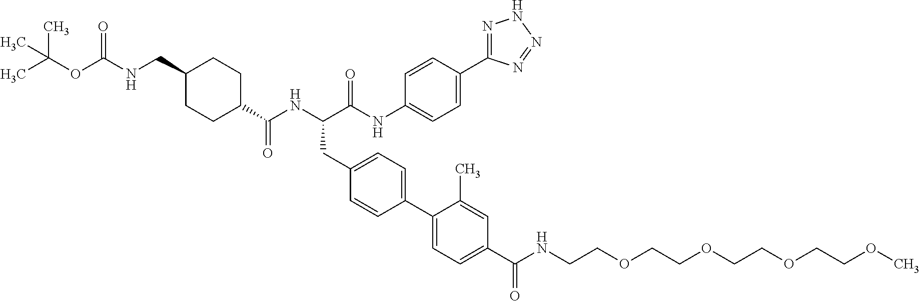

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 36 mg (0.18 mmol) of 2,5,8,11-tetraoxatridecan-13-amine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 80 mg of a mixture of the title compound and the corresponding deprotected amine, which was used directly in the next stage.

LC-MS (Method 1): Rₜ=1.00 min; MS (ESIneg): m/z=869 [M−H]⁻.

### Example 85A

### tert-Butyl {[trans-4-({(2S)-3-[2′-methyl-4′-(2,5,8,11-tetraoxatridecan-13-ylcarbamoyl)biphenyl-4-yl]-1-oxo-1-[(3-oxo-2,3-dihydro-1H-indazol-6-yl)amino]propan-2-yl}carbamoyl)cyclohexyl]-methyl}carbamate

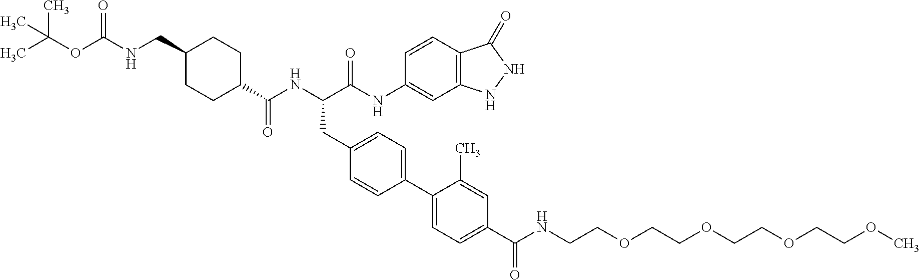

A solution of 100 mg (0.15 mmol) of 4′-{(2S)-2-{[(trans-4-{[(tert-butoxycarbonyl)-amino]methyl}cyclohexyl)carbonyl]amino}-3-oxo-3-[(3-oxo-2,3-dihydro-1H-indazol-6-yl)-amino]propyl}-2-methylbiphenyl-4-carboxylic acid and 37 mg (0.18 mmol) of 2,5,8,11-tetraoxatridecan-13-amine in 5 ml of tetrahydrofuran was admixed with 68 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 64 mg of a mixture of the title compound and the corresponding deprotected amine, which was used directly in the next stage.

LC-MS (Method 1): Rₜ=0.94 min; MS (ESIneg): m/z=857 [M−H]⁻.

### Example 86A

### tert-Butyl [(trans-4-{[(2S)-3-(4′-{[3-(diethylamino)propyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate

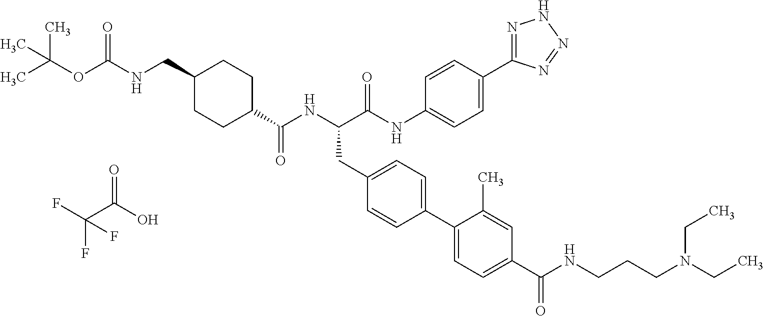

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 23 mg (0.18 mmol) of diethylaminopropylamine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. After addition of 0.4 ml of N,N-dimethylformamide, the mixture was stirred at RT for a further 3 d. The reaction mixture was separated directly by means of preparative HPLC (eluent: acetonitrile/water with 0.1% TFA (gradient)). This gave 84 mg of a mixture of the title compound and the corresponding deprotected amine, which was used directly in the next stage.

LC-MS (Method 1): Rₜ=0.86 min; MS (ESIneg): m/z=792 [M−H-TFA]⁻.

### Example 87A

### tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-chlorobiphenyl-4-yl}carbonyl)amino]-2-methylpiperidine-1-carboxylate

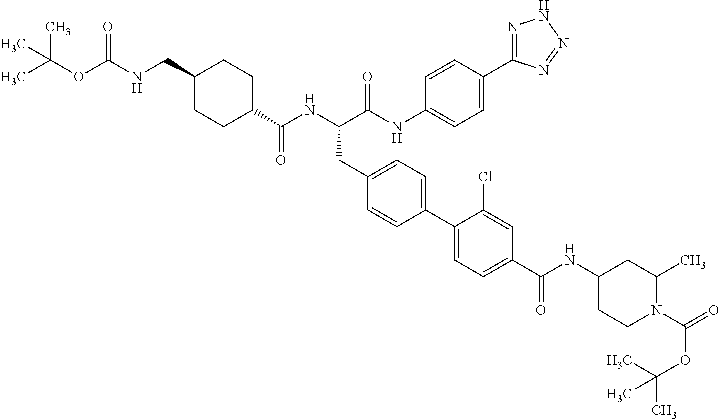

A solution of 100 mg (0.14 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-chlorobiphenyl-4-carboxylic acid and 37 mg (0.17 mmol) of 1-(tert-butoxycarbonyl)-4-amino-2-methylpiperidine in 5 ml of tetrahydrofuran was admixed with 65 mg (0.17 mmol) of N-[(dimethylamino)(3H-[1,2,3]triazolo[4,5-b]pyridin-3-yloxy)methylidene]-N-methylmethanaminium hexafluorophosphate and 0.03 ml (0.17 mmol) of N,N-diisopropylethylamine and stirred at RT for 3 d. The reaction mixture was separated directly by means of preparative HPLC (eluent: acetonitrile/water with 0.1% TFA (gradient)). This gave 71 mg of a mixture of the title compound and the corresponding deprotected amine, which was used directly in the next stage.

LC-MS (Method 1): Rₜ=1.20 min; MS (ESIneg): m/z=896 [M−H]⁻.

### Example 88A

### tert-Butyl 4-{[(4′-{(2S)-2-{[(trans-4-{[(tert-butoxycarbonyl)amino]methyl}cyclohexyl)-carbonyl]amino}-3-oxo-3-[(3-oxo-2,3-dihydro-1H-indazol-6-yl)amino]propyl}-2-methylbiphenyl-4-yl)carbonyl]amino}-2-methylpiperidine-1-carboxylate

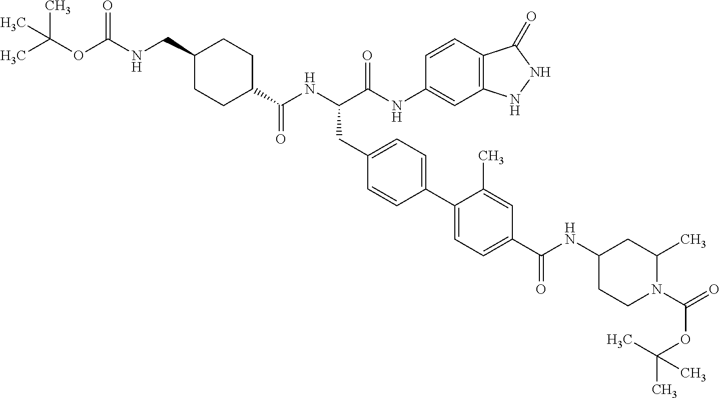

A solution of 100 mg (0.13 mmol) of 4′-[(2S)-2-{[(trans-4-{[(tert-butoxycarbonyl)-amino]methyl}cyclohexyl)carbonyl]amino}-3-oxo-3-[(3-oxo-2,3-dihydro-1H-indazol-6-yl)-amino]propyl}-2-methylbiphenyl-4-carboxylic acid and 33 mg (0.15 mmol) of 1-(tert-butoxycarbonyl)-4-amino-2-methylpiperidine in 5 ml of tetrahydrofuran was admixed with 58 mg (0.15 mmol) of N-[(dimethylamino)(3H-[1,2,3]triazolo[4,5-b]pyridin-3-yloxy)methylidene]-N-methylmethanaminium hexafluorophosphate and 0.03 ml (0.15 mmol) of N,N-diisopropylethylamine and stirred at RT for 3 d. The reaction mixture was separated directly by means of preparative HPLC (eluent: acetonitrile/water with 0.1% TFA (gradient)). This gave 52 mg of a mixture of the title compound and the corresponding deprotected amine, which was used directly in the next stage.

LC-MS (Method 1): Rₜ=1.13 min; MS (ESIneg): m/z=864 [M−H]⁻.

### Example 89A

### tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]-2-methylpiperidine-1-carboxylate

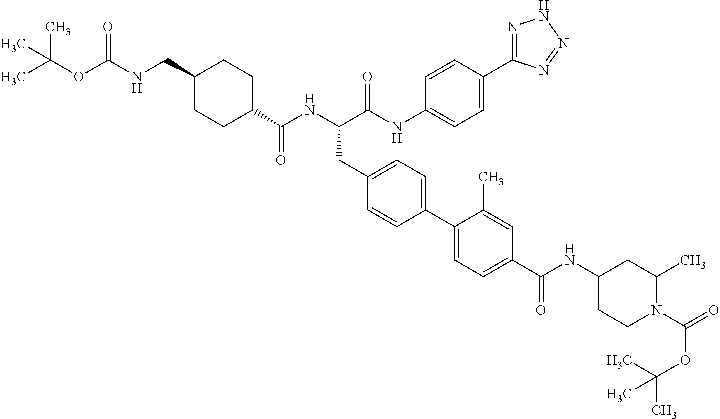

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 38 mg (0.18 mmol) of 1-(tert-butoxycarbonyl)-4-amino-2-methylpiperidine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 3 d. The reaction mixture was separated twice by means of preparative HPLC (eluent: acetonitrile/water with 0.1% TFA (gradient)). This gave 57 mg of a mixture of the title compound and the corresponding deprotected amine, which was used directly in the next stage.

LC-MS (Method 1): Rₜ=1.20 min; MS (ESIneg): m/z=876 [M−H]⁻.

### Example 90A

### tert-Butyl [(trans-4-{[(2S)-3-{4′-[(trans-4-hydroxycyclohexyl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]-carbamate

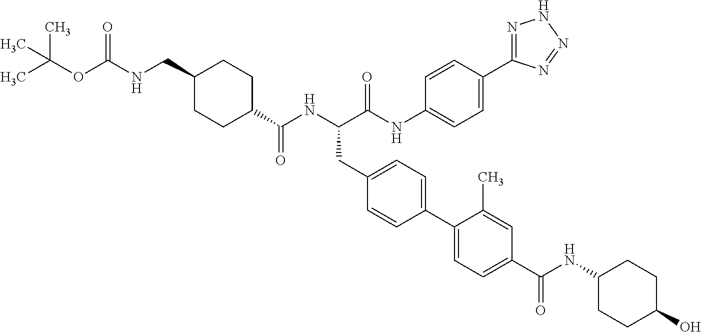

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 20 mg (0.18 mmol) of trans-4-aminocyclohexanol in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 5 h. The reaction mixture was separated directly by means of preparative HPLC (eluent: acetonitrile/water with 0.1% TFA (gradient)). This gave 42 mg of a mixture of the title compound and the corresponding deprotected amine, which was used directly in the next stage.

LC-MS (Method 1): Rₜ=0.96 min; MS (ESIneg): m/z=777 [M−H]⁻.

### Example 91A

### tert-Butyl [(trans-4-{[(2S)-3-[4′-({trans-4-[(tert-butoxycarbonyl)amino]cyclohexyl}carbamoyl)-2′-methylbiphenyl-4-yl]-1-oxo-1-{[4-(2H-tetrazol-5-yl)phenyl]amino}propan-2-yl]carbamoyl}-cyclohexyl)methyl]carbamate

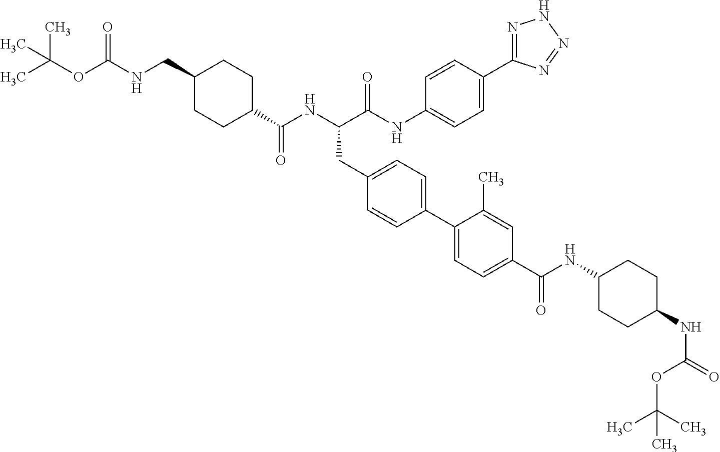

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 38 mg (0.18 mmol) of tert-butyl (trans-4-aminocyclohexyl)carbamate in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 5 h. The reaction mixture was separated directly by means of preparative HPLC (eluent: acetonitrile/water with 0.1% TFA (gradient)). This gave 52 mg of a mixture of the title compound and the corresponding deprotected amine, which was used directly in the next stage.

LC-MS (Method 1): Rₜ=1.14 min; MS (ESIneg): m/z=876 [M−H]⁻.

### Example 92A

### tert-Butyl [(trans-4-{[(2S)-3-(4′-{[3-(dimethylamino)propyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate

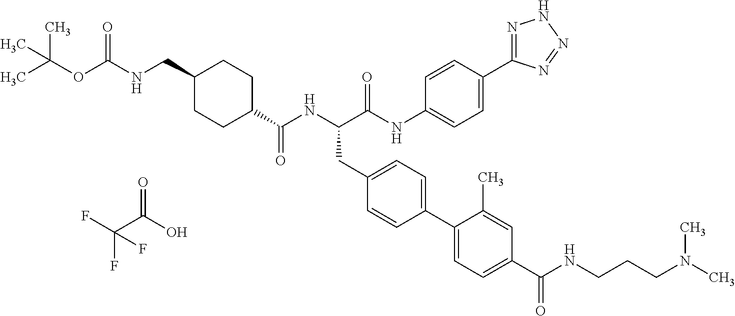

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 18 mg (0.18 mmol) of 3-dimethylamino-1-propylamine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 5 h. After addition of 0.4 ml of N,N-dimethylformamide, the mixture was stirred at RT for a further 16 h. Then 28 mg (0.07 mmol) of N-[(dimethylamino)(3H-[1,2,3]triazolo[4,5-b]pyridin-3-yloxy)methylidene]-N-methylmethanaminium hexafluorophosphate were added and the mixture was stirred at RT for a further 6 h, before the reaction mixture was separated directly by means of preparative HPLC (eluent: acetonitrile/water with 0.1% TFA (gradient)). This gave 86 mg of a mixture of the title compound and the corresponding deprotected amine, which was used directly in the next stage.

LC-MS (Method 1): Rₜ=0.80 min; MS (ESIneg): m/z=764 [M−H-TFA]⁻.

### Example 93A

### tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]biphenyl-4-yl}carbonyl)-amino]piperidine-1-carboxylate

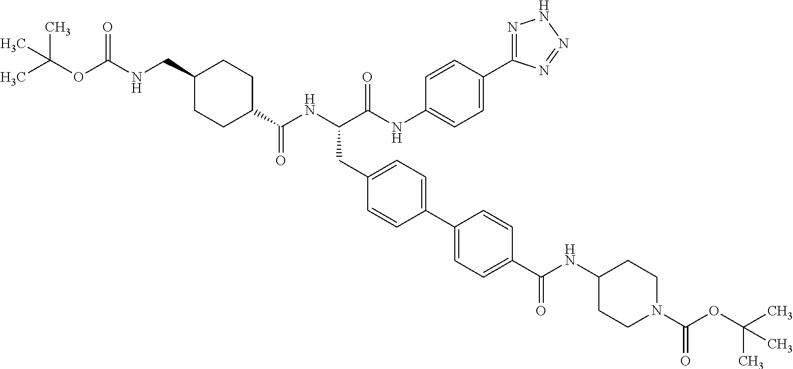

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-biphenyl-4-carboxylic acid and 36 mg (0.18 mmol) of tert-butyl 4-aminopiperidine-1-carboxylate in 5 ml of tetrahydrofuran was admixed with 68 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 104 mg of a mixture of the title compound and the corresponding deprotected amine, which was used directly in the next stage.

LC-MS (Method 1): Rₜ=1.14 min; MS (ESIneg): m/z=848 [M−H]⁻.

### Example 94A

### tert-Butyl [(trans-4-{[(2S)-3-[4′-({2-[diethylamino]ethyl}carbamoyl)biphenyl-4-yl]-1-oxo-1-{[4-(2H-tetrazol-5-yl)phenyl]amino}propan-2-yl]carbamoyl}cyclohexyl)methyl]carbamate trifluoroacetate

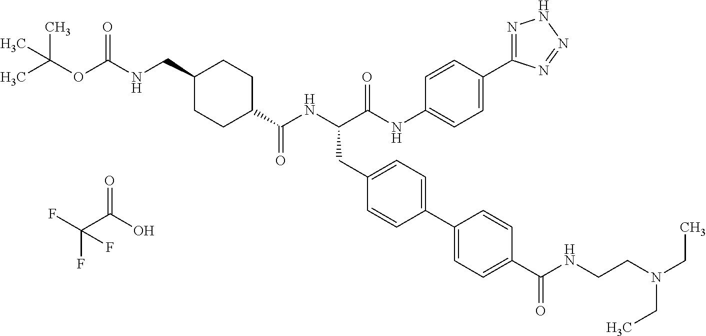

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-biphenyl-4-carboxylic acid and 21 mg (0.18 mmol) of diethylaminopropylamine in 5 ml of tetrahydrofuran was admixed with 68 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 72 mg of a mixture of the title compound and the corresponding deprotected amine, which was used directly in the next stage.

LC-MS (Method 1): Rₜ=0.86 min; MS (ESIneg): m/z=764 [M−H-TFA]⁻.

### Example 95A

### tert-Butyl (3R)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]biphenyl-4-yl}carbonyl)-amino]pyrrolidine-1-carboxylate

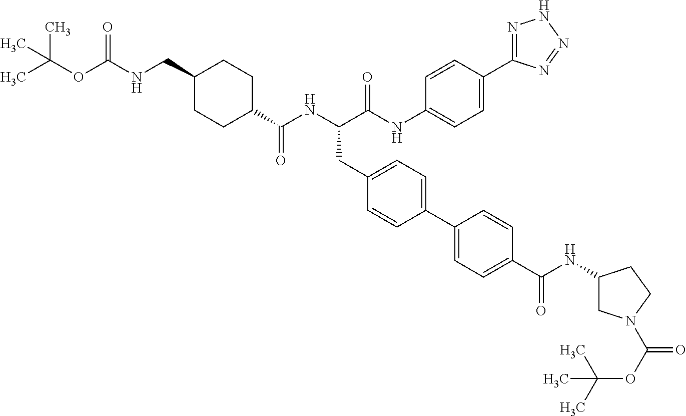

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-biphenyl-4-carboxylic acid and 33 mg (0.18 mmol) of (R)-(+)-1-(tert-butoxycarbonyl)-3-aminopyrrolidine in 5 ml of tetrahydrofuran was admixed with 68 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 81 mg of a mixture of the title compound and the corresponding deprotected amine, which was used directly in the next stage.

LC-MS (Method 1): Rₜ=1.10 min; MS (ESIneg): m/z=834 [M−H]⁻.

### Example 96A

### tert-Butyl [(trans-4-{[(2S)-3-{4′-[(1-isopropylpiperidin-4-yl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate trifluoroacetate

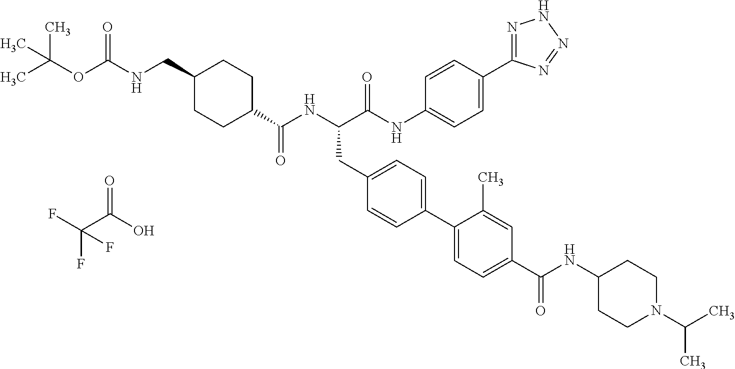

A solution of 100 mg (0.15 mmol) of 4′-[(2S)-2-{[(trans-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 25 mg (0.18 mmol) of 4-amino-1-isopropylpiperidine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. After addition of 0.5 ml of N,N-dimethylformamide, the mixture was stirred at RT for a further 7 h. The reaction mixture was separated directly by means of preparative HPLC (eluent: acetonitrile/water with 0.1% TFA (gradient)). This gave 43 mg of a mixture of the title compound and the corresponding deprotected amine, which was used directly in the next stage.

LC-MS (Method 1): Rₜ=0.83 min; MS (ESIneg): m/z=804 [M−H-TFA]⁻.

### Example 97A

### 2-[({4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}carbonyl)amino]-N,N,N-trimethylethanaminium

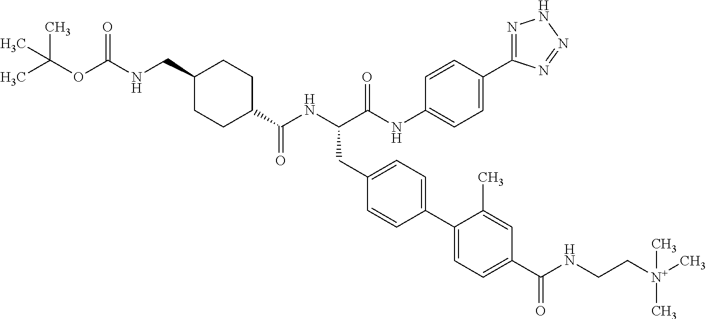

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 25 mg (0.18 mmol) of 2-amino-N,N,N-trimethylethanamonium hydrochloride in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 31 mg of a mixture of the title compound and the corresponding deprotected amine, which was used directly in the next stage.

### Example 98A

### tert-Butyl [(trans-4-{[(2S)-3-{2′-methyl-4′-[(1-methylpiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]carbamate trifluoroacetate

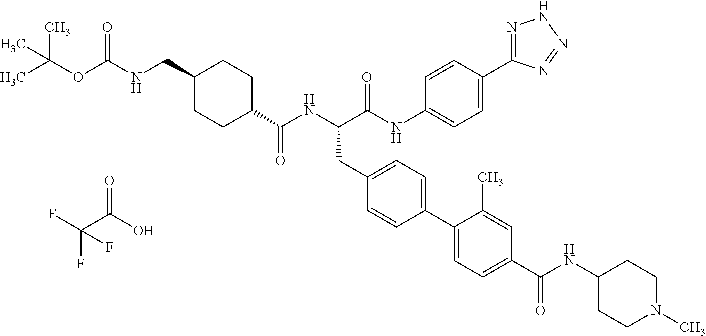

A solution of 100 mg (0.15 mmol) of 4′-[(2S)-2-{[(trans-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 20 mg (0.18 mmol) of 4-amino-1-methylpiperidine in 5 ml of tetrahydrofuran and 0.4 of N,N-dimethylformamide was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 27 mg of a mixture of the title compound and the corresponding deprotected amine, which was used directly in the next stage.

LC-MS (Method 1): Rₜ=0.80 min; MS (ESIneg): m/z=776 [M−H-TFA]⁻.

### Example 99A

### tert-Butyl 6-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]-3-azabicyclo[3.1.0]hexane-3-carboxylate

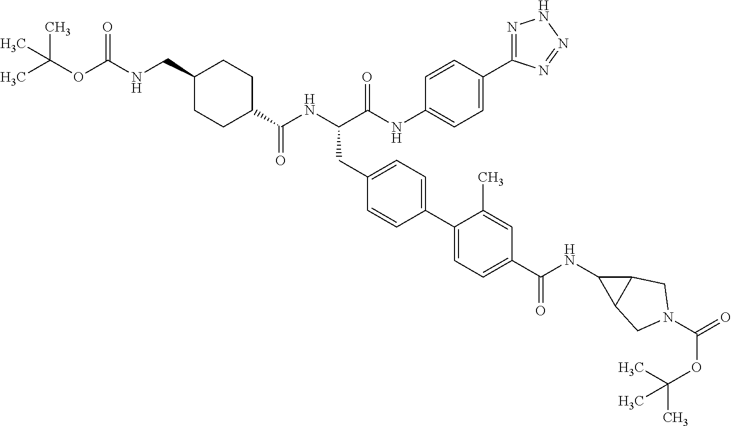

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 35 mg (0.18 mmol) of 3-(tert-butoxycarbonyl)-6-amino-3-azabicyclo[3.1.0]hexane in 5 ml of tetrahydrofuran and 0.4 ml of N,N,-dimethylformamide was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water gradient, 0.1% trifluoroacetic acid). This gave 65 mg of a mixture of the title compound and the corresponding deprotected amine, which was used directly in the next stage.

LC-MS (Method 1): Rₜ=1.13 min; MS (ESIneg): m/z=860 [M−H]⁻.

### Example 100A

### tert-Butyl [(trans-4-{[(2S)-3-{2′-methyl-4′-[(8-methyl-8-azabicyclo[3.2.1]oct-3-yl)carbamoyl]-biphenyl-4-yl}-1-oxo-1-{[4-(2H-tetrazol-5-yl)phenyl]amino}propan-2-yl]carbamoyl}cyclohexyl)-methyl]carbamate trifluoroacetate

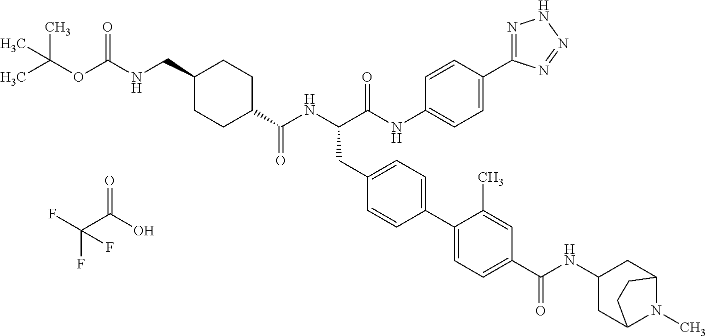

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 25 mg (0.18 mmol) of 8-methyl-8-azabicyclo[3.2.1]octan-3-amine in 5 ml of tetrahydrofuran and 0.4 ml of N,N-dimethylformamide was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 53 mg of a mixture of the title compound and the corresponding deprotected amine, which was used directly in the next stage.

LC-MS (Method 1): Rₜ=0.80 min; MS (ESIneg): m/z=802 [M−H-TFA]⁻.

### Example 101A

### tert-Butyl 4-[({4′-[(2S)-2-{[(trans-4-{[(tert-butoxycarbonyl)amino]methyl}cyclohexyl)-carbonyl]-amino}-3-oxo-3-{[4-(2H-tetrazol-5-yl)phenyl]amino}propyl]-3-fluorobiphenyl-4-yl}carbonyl)-amino]piperidine-1-carboxylate

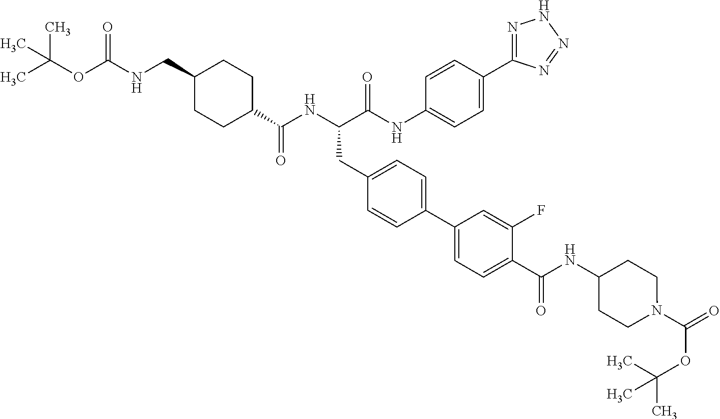

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-3-fluorobiphenyl-4-carboxylic acid and 35 mg (0.18 mmol) of tert-butyl 4-aminopiperidine-1-carboxylate in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. After addition of a further 29 mg (0.15 mmol) of tert-butyl 4-aminopiperidine-1-carboxylate, 55 mg (0.15 mmol) of N-[(dimethyl-amino)(3H-[1,2,3]triazolo[4,5-b]pyridin-3-yloxy)methylidene]-N-methylmethanaminium hexafluorophosphate and 0.03 ml (0.15 mmol) of N,N-diisopropylethylamine, the mixture was stirred once again at RT for 24 h. The reaction mixture was separated directly by means of preparative HPLC (eluent: acetonitrile/water with 0.1% TFA (gradient)). This gave 40 mg of a mixture of the title compound and the corresponding deprotected amine, which was used directly in the next stage.

LC-MS (Method 1): Rₜ=1.16 min; MS (ESIneg): m/z=866 [M−H]⁻.

### Example 102A

### tert-Butyl [(trans-4-{[(2S)-3-(2′-methyl-4′-{[1-(2,2,2-trifluoroethyl)piperidin-4-yl]carbamoyl}-biphenyl-4-yl)-1-oxo-1-{[4-(2H-tetrazol-5-yl)phenyl]amino}propan-2-yl]carbamoyl}cyclohexyl)-methyl]carbamate trifluoroacetate

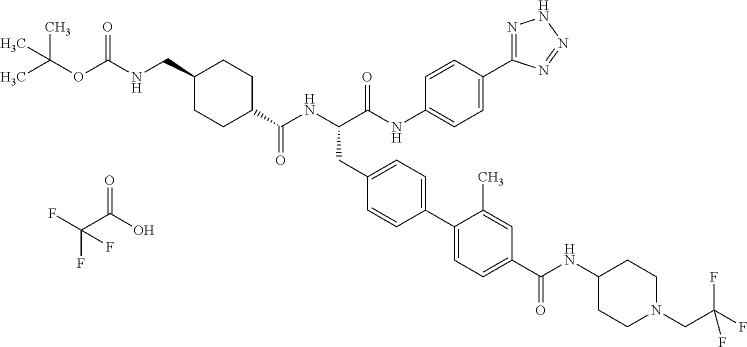

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2-methylbiphenyl-4-carboxylic acid and 32 mg (0.18 mmol) of 1-(2,2,2-trifluoroethyl)piperidin-4-amine in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 47 mg of a mixture of the title compound and the corresponding deprotected amine, which was used directly in the next stage.

LC-MS (Method 1): Rₜ=1.10 min; MS (ESIneg): m/z=844 [M−H-TFA]⁻.

### Example 103A

### tert-Butyl 4-({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]biphenyl-4-yl}-carbonyl)piperazine-1-carboxylate

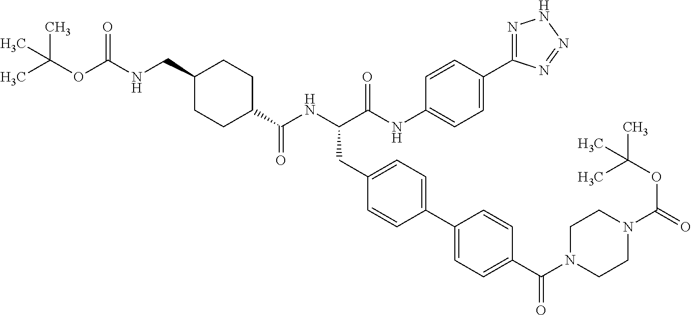

A solution of 100 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-biphenyl-4-carboxylic acid and 33 mg (0.18 mmol) of 1-(tert-butoxycarbonyl)piperazine in 5 ml of tetrahydrofuran was admixed with 68 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 90 mg of a mixture of the title compound and the corresponding deprotected amine, which was used directly in the next stage.

LC-MS (Method 1): Rₜ=1.11 min; MS (ESIneg): m/z=834 [M−H]⁻.

### Example 104A

### tert-Butyl 4-[({4′-[(2S)-2-{[(trans-4-{[(tert-butoxycarbonyl)amino]methyl}cyclohexyl)carbonyl]-amino}-3-oxo-3-({4-[3-(trifluoromethyl)-1H-1,2,4-triazol-5-yl]phenyl}amino)propyl]-2-methyl-biphenyl-4-yl}carbonyl)amino]piperidine-1-carboxylate

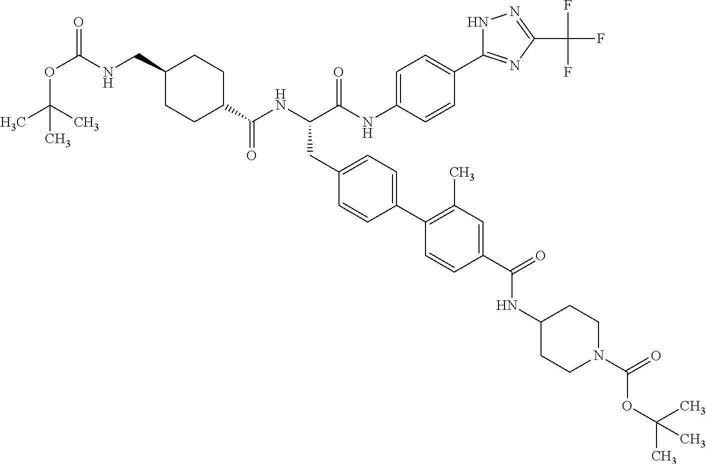

A solution of 125 mg (0.17 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 79 mg (0.35 mmol) of 4-[3-(trifluoromethyl)-1H-1,2,4-triazol-5-yl]aniline in 1.25 ml of dimethylformamide was admixed with 0.09 ml (0.52 mmol) of N,N-diisopropylethylamine and 79 mg (0.21 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction solution was separated by means of preparative HPLC (eluent: methanol/water gradient, 0.01% trifluoroacetic acid). The product-containing fractions were combined and concentrated on a rotary evaporator. The residue was dried under high vacuum. 57 mg (33% of theory, 93% purity) of the title compound were obtained.

¹H NMR (400 MHz, DMSO-d₆): δ=0.68-0.91 (m, 2H), 1.06-1.30 (m, 4H), 1.39 (m, 21H), 1.49-1.83 (m, 7H), 2.06-2.17 (m, 1H), 2.20-2.26 (m, 3H), 2.72-2.77 (m, 2H), 2.90-2.98 (m, 1H), 3.07-3.16 (m, 1H), 3.83-4.03 (m, 2H), 4.66-4.79 (m, 1H), 6.70-6.86 (m, 1H), 7.16-7.30 (m, 4H), 7.35-7.45 (m, 2H), 7.64-7.83 (m, 5H), 7.94-8.04 (m, 2H), 8.13-8.30 (m, 2H), 10.34-10.51 (s, 1H), 15.11-15.24 (s, 1H).

LC-MS (Method 1): Rₜ=1.28 min; MS (ESIneg): m/z=929 [M−H]⁻.

### Example 105A

### tert-Butyl 4-[({4′-[(2S)-2-{[(trans-4-{[(tert-butoxycarbonyl)amino]methyl}cyclohexyl)carbonyl]-amino}-3-{[4-(3-chloro-4H-1,2,4-triazol-5-yl)phenyl]amino}-3-oxopropyl]-2-methylbiphenyl-4-yl}carbonyl)amino]piperidine-1-carboxylate

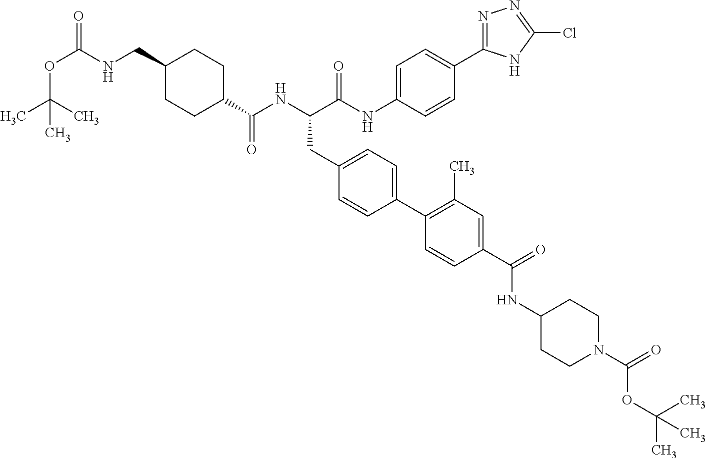

A solution of 250 mg (0.35 mmol) of (2S)-2-{[(trans-4-{[(tert-butoxycarbonyl)-amino]methyl}cyclohexyl)carbonyl]amino}-3-(4′-{[1-(tert-butoxycarbonyl)-piperidin-4-yl]-carbamoyl}-2′-methylbiphenyl-4-yl)propanoic acid and 101 mg (0.52 mmol) of 4-(3-chloro-4H-1,2,4-triazol-5-yl)aniline in 2 ml of dimethylformamide was admixed with 0.18 ml (1.04 mmol) of N,N-diisopropylethylamine and 0.30 ml (0.52 mmol) of a 50% 2,4,6-tripropyl-1,3,5,2,4,6-trioxatriphosphinane 2,4,6-trioxide solution in dimethylformamide and stirred at RT for 16 h. Another 0.15 ml (0.26 mmol) of a 50% 2,4,6-tripropyl-1,3,5,2,4,6-trioxatriphosphinane 2,4,6-trioxide solution in dimethylformamide were added and the mixture was stirred at RT for 2 h. The contents of the flask were diluted with water and the solid formed was filtered off. The mixture was admixed with 10% citric acid solution, extracted three times with ethyl acetate and dried over sodium sulphate. The solvent was removed and the solid formed was dried under high vacuum. 269 mg (73% of theory, 84% purity) of the title compound were obtained.

¹H NMR (400 MHz, DMSO-d₆): δ=0.73-0.91 (m, 2H), 1.04-1.31 (m, 4H), 1.39 (m, 19H), 1.50-1.74 (m, 4H), 1.74-1.83 (m, 2H), 2.05-2.16 (m, 1H), 2.22 (s, 3H), 2.70-2.77 (m, 2H), 2.79-2.98 (m, 3H), 3.06-3.16 (m, 1H), 3.88-4.02 (m, 3H), 4.62-4.81 (m, 1H), 6.73-6.83 (m, 1H), 7.26 (m, 3H), 7.37 (d, 2H), 7.69 (d, 1H), 7.76 (m, 3H), 7.90 (d, 2H), 8.18 (d, 1H), 8.26 (d, 1H), 10.41 (br. s, 1H), 14.70 (br. s, 1H).

LC-MS (Method 1): Rₜ=1.20 min; MS (ESIneg): m/z=895 [M−H]⁻.

### Example 106A

### tert-Butyl 4 {[(4′-{(2S)-2-{[(trans-4-{[(tert-butoxycarbonyl)amino]methyl}cyclohexyl)carbonyl]-amino}-3-[(2-methyl-1H-benzimidazol-6-yl)amino]-3-oxopropyl}-2-methylbiphenyl-4-yl)-carbonyl]amino}piperidine-1-carboxylate

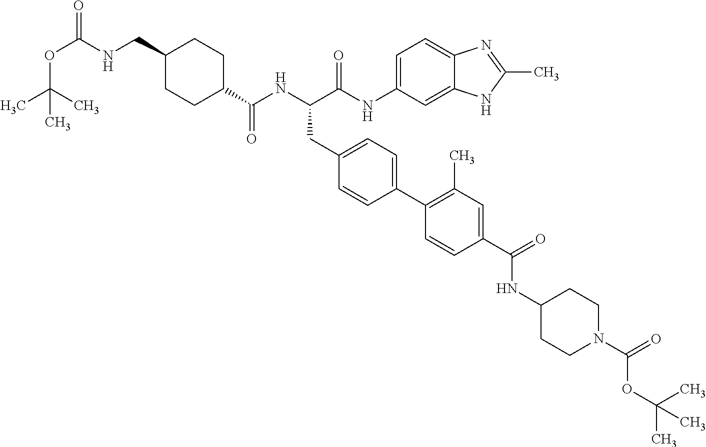

A solution of 75 mg (0.10 mmol) of (2S)-2-{[(trans-4-{[(tert-butoxycarbonyl)-amino]methyl}cyclohexyl)carbonyl]amino}-3-(4′-{[1-(tert-butoxycarbonyl)-piperidin-4-yl]-carbamoyl}-2′-methylbiphenyl-4-yl)propanoic acid and 31 mg (0.21 mmol) of 2-methyl-1H-benzimidazol-5-amine in 1 ml of dichloromethane was admixed with 0.05 ml (0.31 mmol) of N,N-diisopropylamine and 81 mg (0.16 mmol) of (1H-benzotriazol-1-yloxy)tripyrrolidin-1-yl)phosphonium hexafluorophosphate and stirred at RT for 16 h. The reaction solution was separated by means of preparative HPLC (eluent: acetonitrile/water gradient, 0.01% trifluoroacetic acid). The product-containing fractions were combined and concentrated on a rotary evaporator. 23 mg (26% of theory) of the title compound were obtained.

¹H NMR (400 MHz, DMSO-d₆): δ=0.70-0.92 (m, 2H), 1.06-1.30 (m, 4H), 1.39 (m, 20H), 1.49-1.83 (m, 7H), 2.03-2.17 (m, 1H), 2.21 (s, 3H), 2.70-2.83 (m, 5H), 2.88-3.05 (m, 2H), 3.05-3.17 (m, 1H), 3.87-4.01 (m, 3H), 4.67-4.78 (m, 1H), 6.72-6.85 (m, 1H), 7.15-7.31 (m, 3H), 7.37 (d, 2H), 7.48-7.58 (m, 1H), 7.63-7.80 (m, 3H), 8.14-8.33 (m, 3H), 10.40-10.59 (m, 1H), 14.54 (br. s, 1H).

LC-MS (Method 1): Rₜ=1.03 min; MS (ESIpos): m/z=851 [M+H]⁺.

### Example 107A

### tert-Butyl 4-[({4′-[(2S)-2-{[(trans-4-{[(tert-butoxycarbonyl)amino]methyl}cyclohexyl)carbonyl]-amino}-3-oxo-3-{[2-(pyridin-2-yl)-1H-benzimidazol-5-yl]amino}propyl]-2-methylbiphenyl-4-yl}carbonyl)amino]piperidine-1-carboxylate

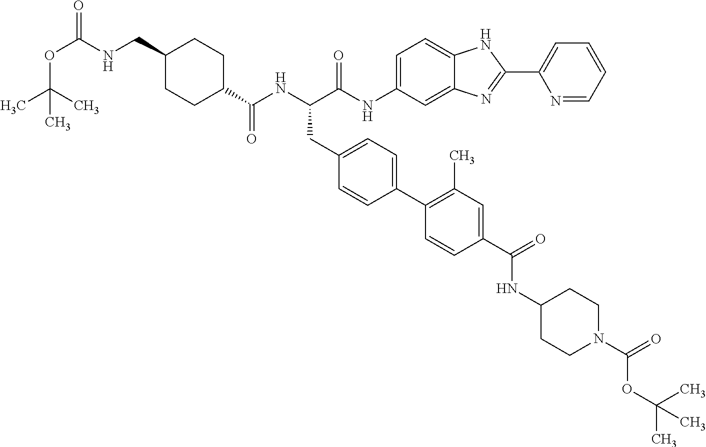

A solution of 75 mg (0.10 mmol) of (2S)-2-{[(trans-4-{[(tert-butoxycarbonyl)-amino]methyl}cyclohexyl)carbonyl]amino}-3-(4′-{[1-(tert-butoxycarbonyl)-piperidin-4-yl]-carbamoyl}-2′-methylbiphenyl-4-yl)propanoic acid and 44 mg (0.21 mmol) of 2-(pyridin-2-yl)-1H-benzimidazol-5-amine in 1 ml of dichloromethane was admixed with 0.05 ml (0.31 mmol) of N,N-diisopropylamine and 81 mg (0.16 mmol) of (1H-benzotriazol-1-yloxy)(tripyrrolidin-1-yl)phosphonium hexafluorophosphate and stirred at RT for 16 h. The reaction solution was separated by means of preparative HPLC (eluent: acetonitrile/water gradient, 0.01% trifluoroacetic acid). The product-containing fractions were combined and concentrated on a rotary evaporator. 49 mg (33% of theory, 63% purity) of the title compound were obtained.

¹H NMR (400 MHz, DMSO-d₆): δ=0.71-0.96 (m, 2H), 1.39 (m, 32H), 2.04-2.18 (m, 2H), 2.22 (s, 3H), 2.69-2.79 (m, 1H), 2.91-3.03 (m, 1H), 3.05-3.19 (m, 1H), 3.26-3.38 (m, 1H), 3.85-4.00 (m, 3H), 4.71-4.81 (m, 1H), 6.70-6.84 (m, 1H), 7.18-7.42 (m, 6H), 7.55 (d, 1H), 7.65-7.83 (m, 4H), 8.08-8.32 (m, 4H), 8.43 (d, 1H), 8.84 (d, 1H), 10.49 (br. s, 1H).

LC-MS (Method 1): Rₜ=1.16 min; MS (ESIpos): m/z=913 [M+H]⁺.

### Example 108A

### tert-Butyl 4-[({4′-[(2S)-2-{[(trans-4-{[(tert-butoxycarbonyl)amino]methyl}cyclohexyl)carbonyl]-amino}-3-oxo-3-{[2-(trifluoromethyl)-1H-benzimidazol-6-yl]amino}propyl]-2-methylbiphenyl-4-yl}carbonyl)amino]piperidine-1-carboxylate

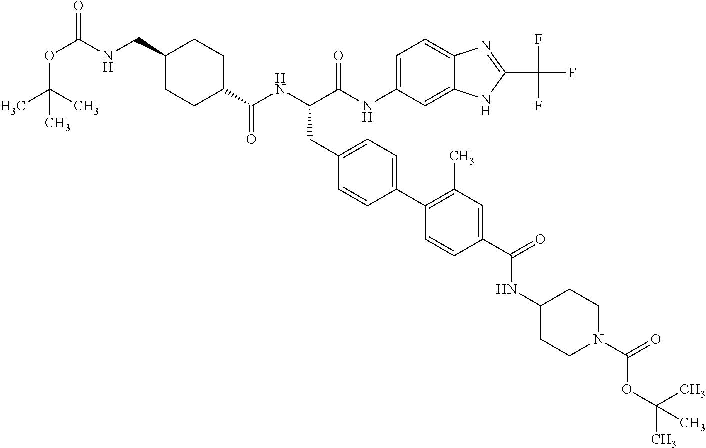

A solution of 73 mg (0.10 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 31 mg (0.15 mmol) of 2-(trifluoromethyl)-1H-benzimidazol-6-amine in 1 ml of N,N-dimethylformamide was admixed with 0.05 ml (0.31 mmol) of N,N-diisopropylamine and 46 mg (0.12 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction solution was separated by means of preparative HPLC (eluent: acetonitrile/water gradient, 0.01% trifluoroacetic acid). The product-containing fractions were combined and concentrated on a rotary evaporator. 66 mg (71% of theory, 88% purity) of the title compound were obtained.

¹H NMR (400 MHz, DMSO-d₆): δ=0.71-0.89 (m, 3H), 1.04-1.30 (m, 7H), 1.31-1.46 (m, 12H), 1.48-1.88 (m, 8H), 2.04-2.26 (m, 5H), 2.74 (m, 5H), 3.17 (s, 2H), 3.96 (s, 2H), 4.67-4.82 (m, 1H), 6.71-6.85 (m, 1H), 7.19-7.29 (m, 3H), 7.31-7.46 (m, 3H), 7.61-7.79 (m, 3H), 8.20 (m, 3H), 10.25-10.38 (s, 1H).

LC-MS (Method 1): Rₜ=1.19 min; MS (ESIpos): m/z=904 [M+H]⁺.

### Example 109A

### tert-Butyl 4-{[(4′-{(2S)-2-{[(trans-4-{[(tert-butoxycarbonyl)amino]methyl}cyclohexyl)carbonyl]-amino}-3-oxo-3-[(2-oxo-2,3-dihydro-1H-benzimidazol-5-yl)amino]propyl}-2-methylbiphenyl-4-yl)carbonyl]amino}piperidine-1-carboxylate

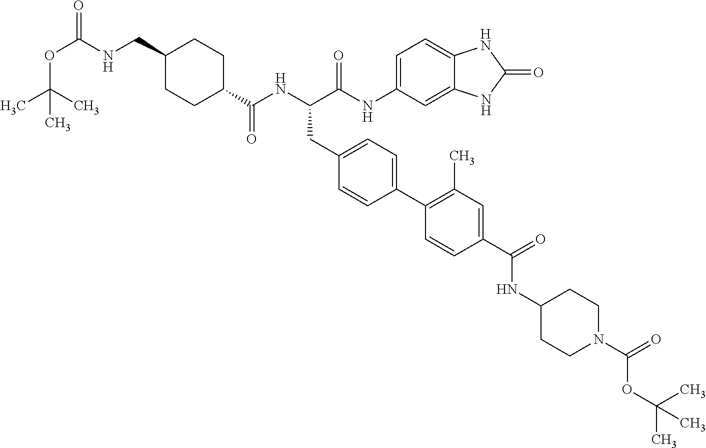 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

A solution of 73 mg (0.10 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 23 mg (0.15 mmol) of 5-amino-1,3-dihydro-2H-benzimidazol-2-one in 0.5 ml of N,N-dimethylformamide was admixed with 0.05 ml (0.31 mmol) of N,N-diisopropylamine and 46 mg (0.12 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction solution was admixed with water; the precipitate formed was filtered off. The precipitate was washed with acetonitrile, dilute sodium hydroxide solution and diethyl ether, and dried under high vacuum. 83 mg (92% of theory) of the title compound were obtained.

¹H NMR (400 MHz, DMSO-d₆): δ=0.81 (m, 2H), 1.05-1.47 (m, 25H), 1.48-1.85 (m, 6H), 2.11 (m, 1H), 2.16-2.27 (m, 3H), 2.69-2.78 (m, 3H), 3.06 (m, 1H), 3.81-4.07 (m, 3H), 4.69 (m, 1H), 6.68-6.87 (m, 2H), 7.02 (dd, 1H), 7.15-7.48 (m, 6H), 7.64-7.80 (m, 2H), 8.11 (d, 1H), 8.26 (d, 1H), 9.97 (s, 1H), 10.53 (br. s, 2H).

LC-MS (Method 1): Rₜ=1.19 min; MS (ESIpos): m/z=904 [M+H]⁺.

### Example 110A

### tert-Butyl 4-[({4′-[(2S)-2-{[(trans-4-{[(tert-butoxycarbonyl)amino]methyl}cyclohexyl)carbonyl]-amino}-3-({4-[3-(methoxymethyl)-4H-1,2,4-triazol-5-yl]phenyl}amino)-3-oxopropyl]-2-methyl-biphenyl-4-yl}carbonyl)amino]piperidine-1-carboxylate

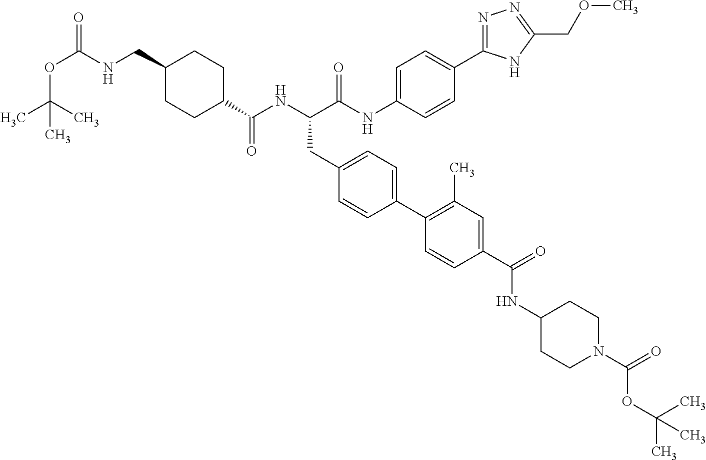

A solution of 125 mg (0.17 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 83 mg (0.35 mmol) of 4-[3-(methoxymethyl)-4H-1,2,4-triazol-5-yl]aniline hydrochloride in 1.25 ml of N,N-dimethylformamide was admixed with 0.09 ml (0.52 mmol) of N,N-diisopropylamine and 79 mg (0.21 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction solution was admixed with water; the precipitate formed was filtered off. The precipitate was washed with acetonitrile, dilute sodium hydroxide solution and diethyl ether, and dried under high vacuum. The residue was dissolved in methanol and separated by means of preparative HPLC (eluent: methanol/water gradient, 0.01% trifluoroacetic acid). The product-containing fractions were combined and concentrated on a rotary evaporator. 43 mg (24% of theory, 89% purity) of the title compound were obtained.

¹H NMR (400 MHz, DMSO-d₆): δ=0.70-0.93 (m, 2H), 1.04-1.83 (m, 30H), 2.04-2.18 (m, 1H), 2.22 (s, 3H), 2.74 (m, 6H), 3.05-3.19 (m, 1H), 3.34 (s, 3H), 3.90-4.00 (m, 1H), 4.43-4.59 (m, 2H), 4.69-4.79 (m, 1H), 6.71-6.82 (m, 1H), 7.17-7.42 (m, 6H), 7.65-7.77 (m, 4H), 7.93 (d, 2H), 8.15 (d, 1H), 8.27 (d, 1H), 10.30 (s, 1H).

LC-MS (Method 1): Rₜ=1.14 min; MS (ESIpos): m/z=907 [M+H]⁺.

### Example 111A

### tert-Butyl 4-{[(4′-{(2S)-2-{[(trans-4-{[(tert-butoxycarbonyl)amino]methyl}cyclohexyl)carbonyl]-amino}-3-oxo-3-[(2-oxo-2,3-dihydro-1,3-benzoxazol-6-yl)amino]propyl}-2-methylbiphenyl-4-yl)carbonyl]amino}piperidine-1-carboxylate

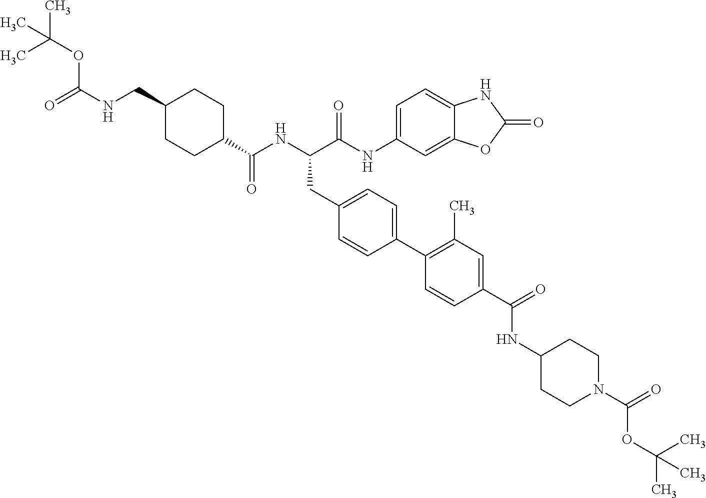

A solution of 100 mg (0.14 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 42 mg (0.28 mmol) of 6-amino-1,3-benzoxazol-2(3H)-one in 1 ml of dimethylformamide was admixed with 0.07 ml (0.42 mmol) of N,N-diisopropylethylamine and 63 mg (0.17 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and the solid formed was filtered off. The solid was washed with 0.5N sodium hydroxide solution, water and diethyl ether and the solid was dried under high vacuum. 109 mg (84% of theory, 91% purity) of the title compound were obtained.

¹H NMR (400 MHz, DMSO-d₆): δ=0.82 (br. s., 3H), 1.04 (d, 4H), 1.08-1.27 (m, 2H), 1.39 (d, 18H), 1.57-1.86 (m, 3H), 2.11 (br. s., 1H), 2.17-2.28 (m, 4H), 2.67-2.99 (m, 5H), 3.04-3.14 (m, 1H), 3.96 (br. s., 4H), 4.70 (d, 1H), 6.67-6.85 (m, 1H), 7.08-7.42 (m, 7H), 7.59 (s, 1H), 7.67-7.84 (m, 3H), 7.95 (s, 1H), 10.21 (br. s., 1H), 11.47 (br. s, 1H).

LC-MS (Method 1): Rₜ=1.17 min; MS (ESIneg): m/z=851 [M−H]⁻.

### Example 112A

### tert-Butyl 4-[({4′-[(2S)-2-{[(trans-4-{[(tert-butoxycarbonyl)amino]methyl}cyclohexyl)carbonyl]-amino}-3-{[4-(3-methyl-4H-1,2,4-triazol-5-yl)phenyl]amino}-3-oxopropyl]-2-methylbiphenyl-4-yl}carbonyl)amino]piperidine-1-carboxylate

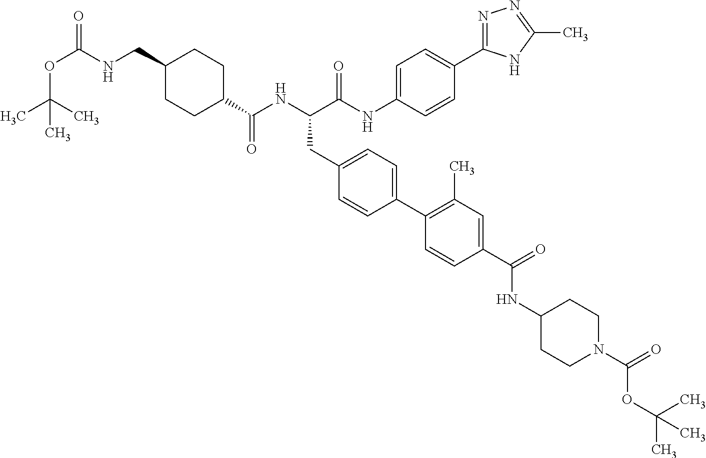

A solution of 73 mg (0.1 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 26 mg (0.15 mmol) of 4-(3-methyl-4H-1,2,4-triazol-5-yl)aniline in 1.1 ml dimethylformamide was admixed with 0.05 ml (0.3 mmol) of N,N-diisopropylethylamine and 46 mg (0.1 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were separated by means of preparative HPLC (eluent: acetonitrile/water gradient, 0.01% trifluoroacetic acid). The product-containing fractions were combined, concentrated on a rotary evaporator, and the residue was dried under high vacuum. 25 mg (27% of theory) of the title compound were obtained.

LC-MS (Method 1): Rₜ=1.13 min; MS (ESIneg): m/z=875 [M−H]⁻.

### Example 113A

tert-Butyl 4-[({4′-[(2S)-2-{[(trans-4-{[(tert-butoxycarbonyl)amino]methyl}cyclohexyl)carbonyl]-amino}-3-oxo-3-{[2-(pentafluoroethyl)-1H-benzimidazol-5-yl]amino}propyl]-2-methylbiphenyl-4-yl}carbonyl)amino]piperidine-1-carboxylate

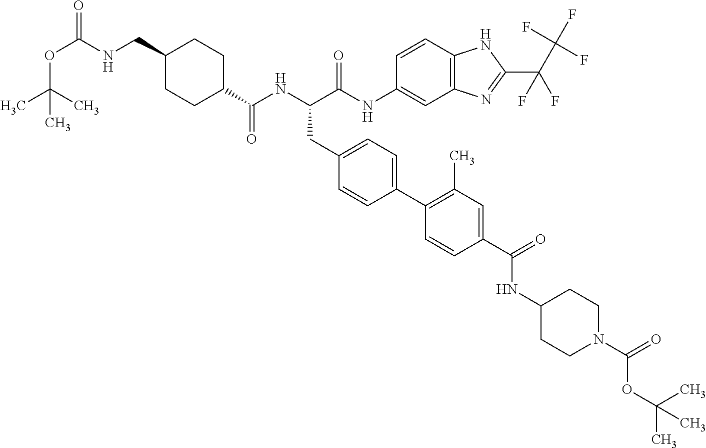

A solution of 100 mg (0.14 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 79 mg (0.28 mmol) of 2-(pentafluoroethyl)-1H-benzimidazol-5-amine hydrochloride in 1 ml of dimethylformamide was admixed with 0.07 ml (0.4 mmol) of N,N-diisopropylethylamine and 63 mg (0.16 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were separated by means of preparative HPLC (eluent: methanol/water gradient, 0.01% trifluoroacetic acid). The product-containing fractions were combined, concentrated on a rotary evaporator, and the residue was dried under high vacuum. 87 mg (65% of theory) of the title compound were obtained.

¹H NMR (400 MHz, DMSO-d₆): δ=0.72-0.88 (m, 2H), 1.01-1.28 (m, 5H), 1.33-1.46 (m, 18H), 1.50-1.86 (m, 7H), 2.07-2.16 (m, 1H), 2.21 (s, 3H), 2.69-2.77 (m, 2H), 2.90-3.01 (m, 1H), 3.06-3.18 (m, 1H), 3.83-4.05 (m, 4H), 4.65-4.86 (m, 1H), 6.68-6.85 (m, 1H), 7.15-7.29 (m, 4H), 7.37 (m, 3H), 7.63-7.76 (m, 3H), 8.07-8.14 (m, 1H), 8.17-8.31 (m, 2H), 10.27 (s, 1H).

LC-MS (Method 1): Rₜ=1.26 min; MS (ESIneg): m/z=952 [M−H]⁻.

### Example 114A

### tert-Butyl 4-{[(4′-{(2S)-2-{[(trans-4-{[(tert-butoxycarbonyl)amino]methyl}cyclohexyl)carbonyl]-amino}-3-[(2-isobutyl-1H-benzimidazol-5-yl)amino]-3-oxopropyl}-2-methylbiphenyl-4-yl)-carbonyl]amino}piperidine-1-carboxylate

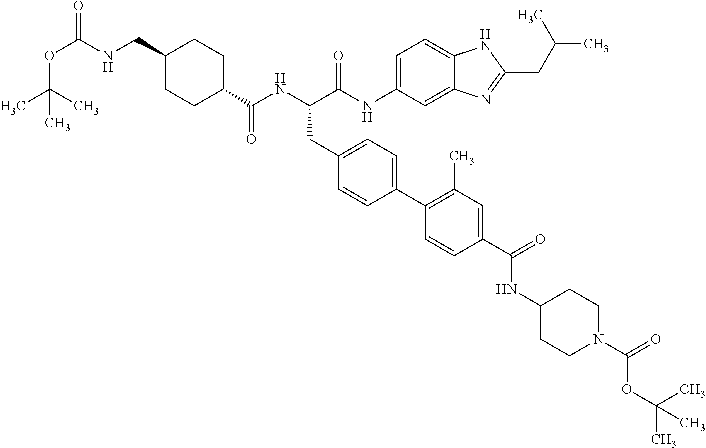 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc3[nH]c(CC(C)C)nc3c2)cc1]

A solution of 100 mg (0.14 mmol) of (2S)-2-[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 53 mg (0.28 mmol) of 2-isobutyl-1H-benzimidazol-5-amine in 1 ml dimethylformamide was admixed with 72 μl (0.42 mmol) of N,N-diisopropylethylamine and 83 mg (0.42 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were admixed with water. The residue was washed with dilute sodium hydroxide solution and diethyl ether, and dried under high vacuum. 134 mg (100% of theory) of the title compound were obtained.

LC-MS (Method 1): Rₜ=0.99 min; MS (ESIneg): m/z=892 [M−H]⁻.

### Example 115A

### tert-Butyl 4-[({4′-[(2S)-2-{[(trans-4-{[(tert-butoxycarbonyl)amino]methyl}cyclohexyl)carbonyl]-amino}-3-oxo-3-{[2-(pyridin-3-yl)-1H-benzimidazol-5-yl]amino}propyl]-2-methylbiphenyl-4-yl}carbonyl)amino]piperidine-1-carboxylate

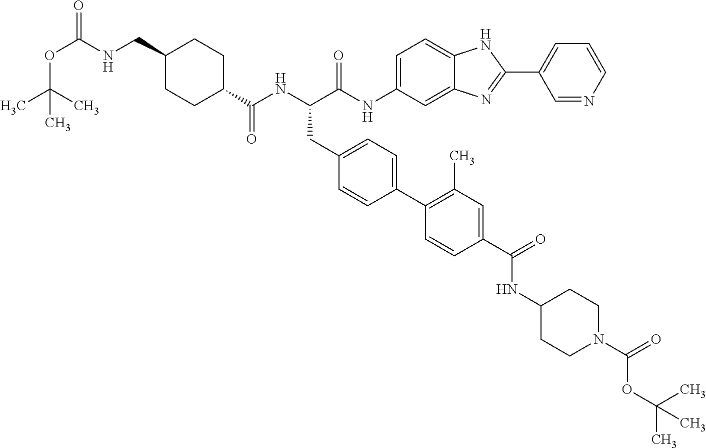 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc3[nH]c(-c4cccnc4)nc3c2)cc1]

A solution of 100 mg (0.14 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 58 mg (0.28 mmol) of 2-(pyridin-3-yl)-1H-benzimidazol-5-amine in 1 ml dimethylformamide was admixed with 72 μl (0.42 mmol) of N,N-diisopropylethylamine and 63 mg (0.16 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were admixed with water. The residue was washed with dilute sodium hydroxide solution, water and diethyl ether, and dried under high vacuum. 153 mg (100% of theory) of the title compound were obtained.

LC-MS (Method 1): Rₜ=1.10 min; MS (ESIneg): m/z=913 [M−H]⁻.

### Example 116A

### tert-Butyl 4-[({4′-[(2S)-2-{[(trans-4-{[(tert-butoxycarbonyl)amino]methyl}cyclohexyl)carbonyl]-amino}-3-oxo-3-{[2-(1H-pyrazol-1-yl)-1H-benzimidazol-5-yl]amino}propyl]-2-methylbiphenyl-4-yl}carbonyl)amino]piperidine-1-carboxylate

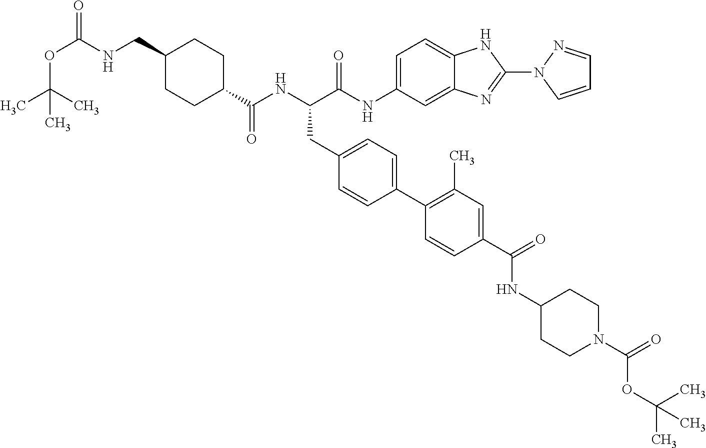 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc3[nH]c(-n4cccn4)nc3c2)cc1]

A solution of 100 mg (0.14 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 55 mg (0.28 mmol) of 2-(1H-pyrazol-1-yl)-1H-benzimidazol-5-amine in 1 ml of dimethylformamide was admixed with 72 μl (0.42 mmol) of N,N-diisopropylethylamine and 63 mg (0.16 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were admixed with water. The residue was washed with dilute sodium hydroxide solution, water and diethyl ether, and dried under high vacuum. 159 mg (100% of theory, 94% purity) of the title compound were obtained.

LC-MS (Method 1): Rₜ=1.19 min; MS (ESIneg): m/z=902 [M−H]⁻.

### Example 117A

### tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2′-fluoro-2-methylbiphenyl-4-yl}carbonyl)amino]piperidine-1-carboxylate

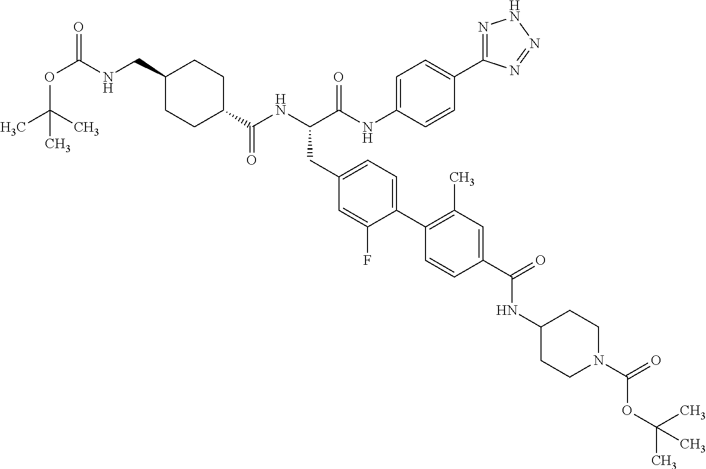 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1F]

A solution of 103 mg (0.15 mmol) of 4′-[(2S)-2-{[(trans-4-{[(tert-butoxycarbonyl)-amino]methyl}cyclohexyl)carbonyl]amino}-3-oxo-3-{[4-(2H-tetrazol-5-yl)phenyl]amino}propyl]-2′-fluoro-2-methylbiphenyl-4-carboxylic acid and 35 mg (0.18 mmol) of tert-butyl 4-aminopiperidine-1-carboxylate in 5 ml of tetrahydrofuran was admixed with 67 mg (0.18 mmol) of N-[(dimethylamino)(3H-[1,2,3]triazolo[4,5-b]pyridin-3-yloxy)methylidene]-N-methylmethanaminium hexafluorophosphate and 0.03 ml (0.18 mmol) of N,N-diisopropylethylamine and stirred at RT for 16 h. The reaction mixture was separated directly by means of preparative HPLC (eluent: acetonitrile/water with 0.1% TFA (gradient)). This gave 85 mg of a mixture of the title compound and the corresponding deprotected amine, which was used directly in the next stage.

LC-MS (Method 1): Rₜ=1.16 min; MS (ESIneg): m/z=880 [M−H]⁻.

### Example 118A

### tert-Butyl [(trans-4-{[(2S)-3-[4′-(cyclopropylcarbamoyl)biphenyl-4-yl]-1-oxo-1-{[4-(1H-tetrazol-5-yl)phenyl]amino}propan-2-yl]carbamoyl}cyclohexyl)methyl]carbamate

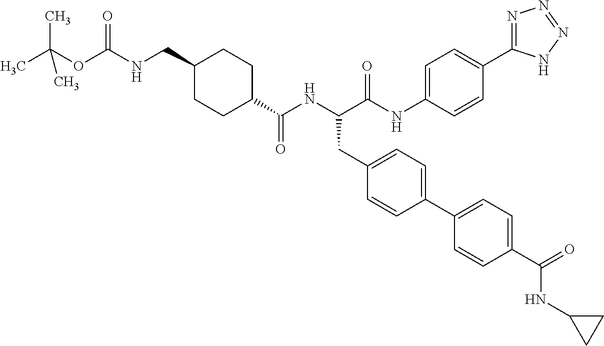

4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [4-(cyclopropyl-carbamoyl)phenyl]boronic acid (74 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 120 min, cooled, filtered and purified by chromatography via HPLC (Method 11). This gave 40 mg (24% of theory) of the title compound.

LC-MS (Method 1): Rₜ=0.82 min; MS (ESIpos): m/z=748.4 [M+H]⁺.

### Example 119A

### tert-Butyl [(trans-4-{[(2S)-3-4′-{[4-(dimethylamino)cyclohexyl]carbamoyl}-2′-methylbiphenyl-4-yl)-1-{[3-fluoro-4-(2H-tetrazol-5-yl)phenyl]amino}-1-oxopropan-2-yl]carbamoyl}cyclohexyl)-methyl]carbamate

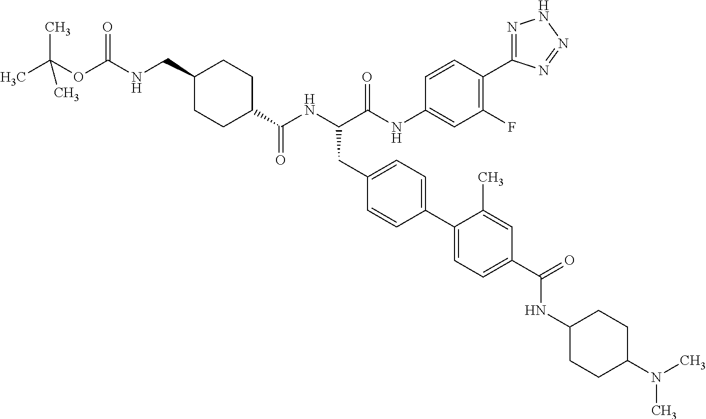

A solution of 150 mg (0.21 mmol) of 4′-[(2S)-2-{[(trans-4-{[(tert-butoxycarbonyl)amino]-methyl}cyclohexyl)carbonyl]amino}-3-{[3-fluoro-4-(2H-tetrazol-5-yl)phenyl]amino}-3-oxopropyl]-2-methylbiphenyl-4-carboxylic acid and 61 mg (0.43 mmol) of N,N-dimethylcyclohexane-1,4-diamine in 2.0 ml of dimethylformamide were admixed with 0.11 ml (0.64 mmol) of N,N-diisopropylethylamine and 163 mg (0.43 mmol) of N-[(dimethylamino)(3H-[1,2,3]triazolo[4,5-b]pyridin-3-yloxy)methylidene]-N-methylmethanaminium hexafluorophosphate. The mixture was stirred at 30° C. for 48 h. The reaction mixture was separated by chromatography via HPLC (Method 11). The product-containing fractions were combined and lyophilized. This gave 37 mg (21% of theory) of the title compound.

LC-MS (Method 5): Rₜ=0.92 min; MS (ESIpos): m/z=824.5 [M+H]⁺.

### Example 120A

### tert-Butyl [(trans-4-{[(2S)-3-(2′-methyl-4′-{[(3S)-2-oxoazepan-3-yl]-carbamoyl}biphenyl-4-yl)-1-oxo-1-{[4-(2H-tetrazol-5-yl)phenyl]amino}propan-2-yl]carbamoyl}-cyclohexyl)methyl]carbamate

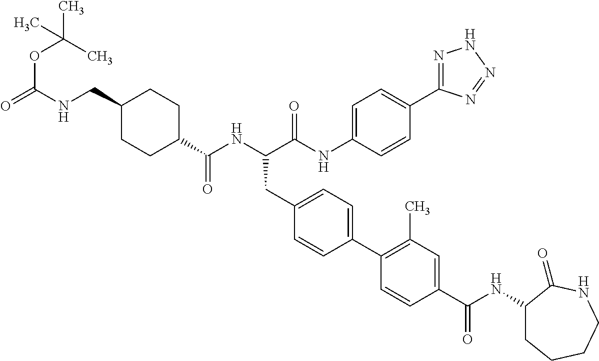

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 37.6 mg (0.3 mmol) of (S)-3-aminohexahydro-2H-azepin-2-one in 1.25 ml of dimethylformamide was admixed with 77 μl (0.4 mmol) of N,N-diisopropylethylamine and 83.7 mg (0.2 mmol) of N-[(dimethylamino)(3H-[1,2,3]triazolo[4,5-b]pyridin-3-yloxy)methylidene]-N-methyl-methanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 74 mg (47% of theory, 84% purity) of the title compound were obtained.

LC-MS (Method 1): Rₜ=1.00 min; MS (ESIneg): m/z=792 [M−H]⁻.

### Example 121A

### 4-(5-{[(2S)-2-{[(trans-4-{[(tert-Butoxycarbonyl)amino]methyl}cyclohexyl)carbonyl]amino}-3-(4′-{[1-(tert-butoxycarbonyl)piperidin-4-yl]carbamoyl}-2′-methylbiphenyl-4-yl)propanoyl]amino}-1H-benzimidazol-2-yl)-2,2,3,3,4,4-hexafluorobutanoic acid

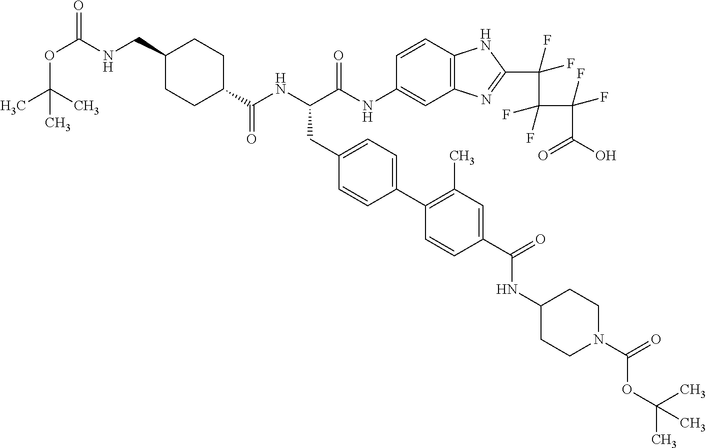 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc3[nH]c(C(F)(F)C(F)(F)C(F)(F)C(=O)O)nc3c2)cc1]

A solution of 275 mg (0.38 mmol) of (2S)-2-{[(trans-4-{[(tert-butoxycarbonyl)-amino]methyl}cyclohexyl)carbonyl]amino}-3-(4-{[1-(tert-butoxycarbonyl)-piperidin-4-yl]-carbamoyl}-2′-methylbiphenyl-4-yl)propanoic acid and 250 mg (0.76 mmol) of 4-(5-amino-1H-benzimidazol-2-yl)-2,2,3,3,4,4-hexafluorobutanoic acid in 5 ml of dimethylformamide was admixed with 0.27 ml (1.52 mmol) of N,N-diisopropylethylamine and 174 mg (0.46 mmol) of HATU and stirred at RT for 3 d. The reaction solution was separated by means of preparative HPLC (eluent: acetonitrile/water gradient, 0.01% trifluoroacetic acid). The product-containing fractions were combined and concentrated on a rotary evaporator. The residue was dried under high vacuum. 75 mg (75% of theory) of the title compound were obtained.

LC-MS (Method 1): Rₜ=1.18 min; MS (ESIneg): m/z=1030 [M−H]⁻.

### Example 122A

### tert-Butyl [(trans-4-{[(2S)-3-(4′-{[1,3-bis(dimethylamino)propan-2-yl]carbamoyl}-2′-methyl-biphenyl-4-yl)-1-oxo-1-{[4-(2H-tetrazol-5-yl)phenyl]amino}propan-2-yl]carbamoyl}cyclohexyl)-methyl]carbamate trifluoroacetate

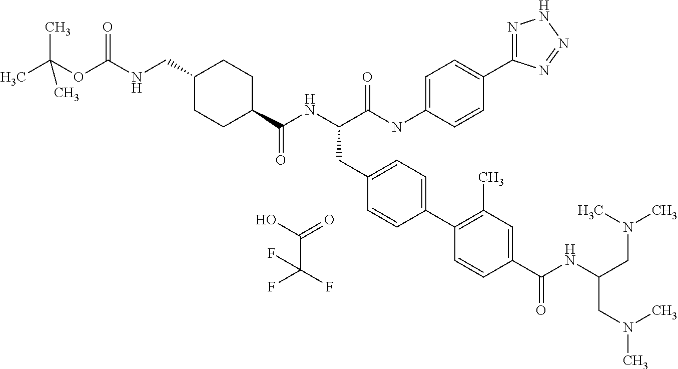

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 42.6 mg (0.29 mmol) of N′,N′,N′,N′-tetramethylpropane-1,2,3-triamine in 1.25 ml of dimethylformamide was admixed with 77 μl (0.44 mmol) of N,N-diisopropylethylamine and 83.7 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 72 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 106 mg (56% of theory, 72% purity) of the title compound were obtained.

LC-MS (Method 1): Rₜ=0.70 min; MS (ESIneg): m/z=809 [M−H]⁻.

### Example 123A

### tert-Butyl [(trans-4-{[(2S)-3-(4′-{[4-(dimethylamino)cyclohexyl]carbamoyl}-2′-methoxybiphenyl-4-yl)-1-oxo-1-{[4-(2H-tetrazol-5-yl)phenyl]amino}propan-2-yl]carbamoyl}cyclohexyl)methyl]-carbamate trifluoroacetate

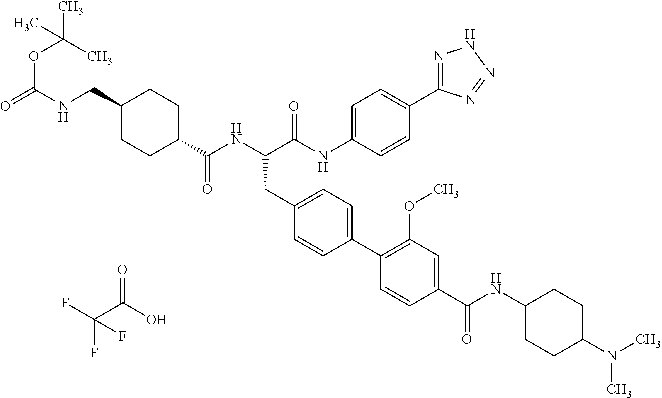

A solution of 100 mg (0.14 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methoxybiphenyl-4-carboxylic acid and 24.5 mg (0.17 mmol) of N,N-dimethylcyclohexane-1,4-diamine in 1 ml of dimethylformamide and 2 ml of tetrahydrofuran was admixed with 30 μl (0.17 mmol) of N,N-diisopropylethylamine and 65.4 mg (0.17 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 68 mg (49% of theory) of the title compound were obtained.

LC-MS (Method 1): Rₜ=0.81 min; MS (ESIneg): m/z=822 [M−H]⁻.

### Example 124A

### tert-Butyl {[trans-4-({(2S)-3-(4′-{[4-(dimethylamino)cyclohexyl]carbamoyl}-2′-methylbiphenyl-4-yl)-1-oxo-1-[(3-oxo-2,3-dihydro-1H-indazol-6-yl)amino]propan-2-yl}carbamoyl)cyclohexyl]-methyl}carbamate trifluoroacetate

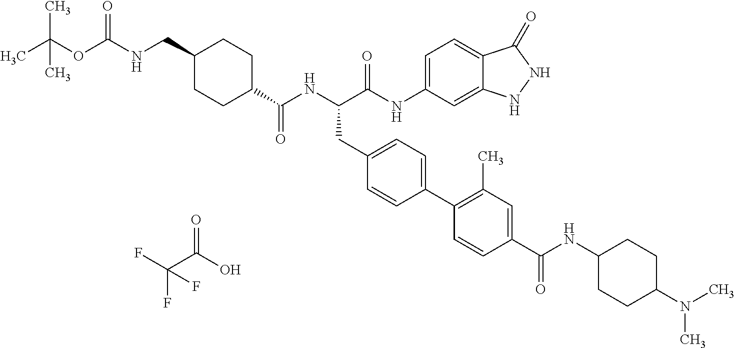

A solution of 66 mg (0.1 mmol) of 4′-{(2S)-2-{[(trans-4-{[(tert-butoxycarbonyl)amino]-methyl}cyclohexyl)carbonyl]amino}-3-oxo-3-[(3-oxo-2,3-dihydro-1H-indazol-6-yl)amino]propyl}-2-methylbiphenyl-4-carboxylic acid and 16.8 mg (0.12 mmol) of N,N-dimethylcyclohexane-1,4-diamine in 1 ml of dimethylformamide and 1 ml of tetrahydrofuran was admixed with 21 μl (0.12 mmol) of N,N-diisopropylethylamine and 45.0 mg (0.12 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 82 mg (73% of theory, 79% purity) of the title compound were obtained.

LC-MS (Method 1): Rₜ=0.79 min; MS (ESIneg): m/z=794 [M−H]⁻.

### Example 125A

### tert-Butyl (trans-4-{[(2S)-3-{4′-[(1,1-dioxidothiomorpholin-4-yl)carbonyl]-2′-methylbiphenyl-4-yl}-1-oxo-1-[4-(2H-tetrazol-5-yl)phenyl]amino}propan-2-yl]carbamoyl}cyclohexyl)methyl]-carbamate

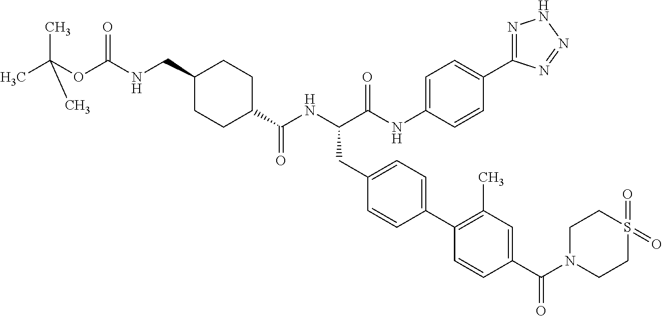

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 39.7 mg (0.29 mmol) of thiomorpholine 1,1-dioxide in 1.25 ml of dimethylformamide was admixed with 77 μl (0.44 mmol) of N,N-diisopropylethylamine and 83.7 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 81 mg (53% of theory, 88% purity) of the title compound were obtained.

LC-MS (Method 1): Rₜ=0.97 min; MS (ESIneg): m/z=799 [M−H]⁻.

### Example 126A

### tert-Butyl [(trans-4-{[(2S)-3-(4′-{[4-(dimethylamino)cyclohexyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]-carbamate trifluoroacetate

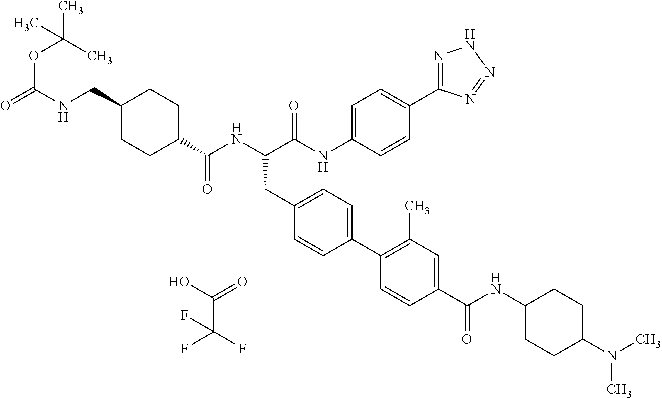

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 41.7 mg (0.29 mmol) of N,N-dimethylcyclohexane-1,4-diamine in 1.25 ml of dimethylformamide was admixed with 77 μl (0.44 mmol) of N,N-diisopropylethylamine and 83.7 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 74 mg (50% of theory, 92% purity) of the title compound were obtained.

LC-MS (Method 1): Rₜ=0.81 min; MS (ESIneg): m/z=806 [M−H]⁻.

### Example 127A

### tert-Butyl (3R)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methoxybiphenyl-4-yl}carbonyl)amino]pyrrolidine-1-carboxylate

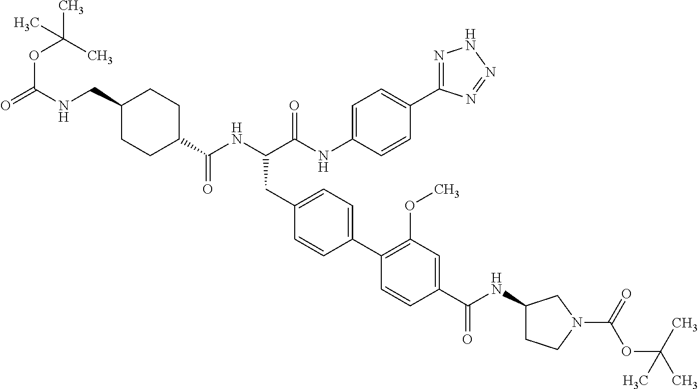

A solution of 100 mg (0.14 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methoxybiphenyl-4-carboxylic acid and 32.05 mg (0.17 mmol) of tert-butyl (3R)-3-aminopyrrolidine-1-carboxylate in 1 ml of dimethylformamide and 2 ml of tetrahydrofuran was admixed with 30 μl (0.17 mmol) of N,N-diisopropylethylamine and 65.4 mg (0.17 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 65 mg (44% of theory, 94% purity) of the title compound were obtained.

LC-MS (Method 1): Rₜ=1.11 min; MS (ESIneg): m/z=866 [M−H]⁻.

### Example 128A

### tert-Butyl [(trans-4-{[(2S)-3-{4′-[(1,3-dihydroxypropan-2-yl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate

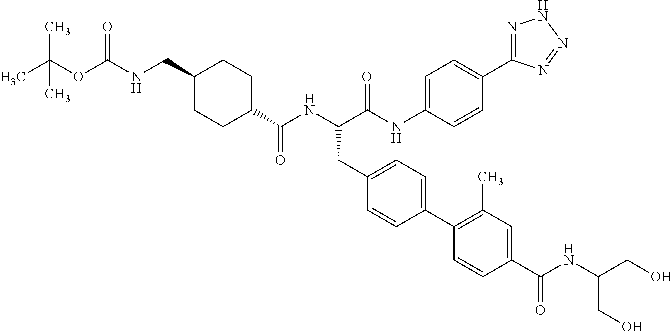

A solution of 100 mg (0.15 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 26.7 mg (0.29 mmol) of 2-amino-1,3-propanediol in 1.25 ml of dimethylformamide was admixed with 77 μl (0.44 mmol) of N,N-diisopropylethylamine and 83.7 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 72 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 37 mg (13% of theory, 46% purity) of the title compound were obtained.

LC-MS (Method 1): Rₜ=0.87 min; MS (ESIneg): m/z=755 [M−H]⁻.

### Example 129A

### tert-Butyl [(trans-4-{[(2S)-3-(2′-methyl-4′-{[(3S)-1-methylpiperidin-3-yl]carbamoyl}biphenyl-4-yl)-1-oxo-1-{[4-(2H-tetrazol-5-yl)phenyl]amino}propan-2-yl]carbamoyl}cyclohexyl)methyl]-carbamate trifluoroacetate

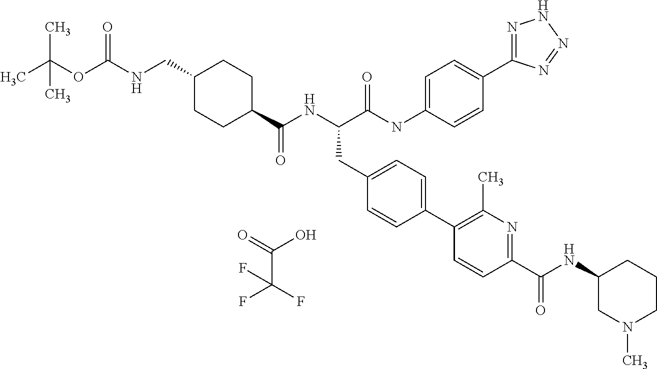 [SMILES: Cc1nc(C(=O)N[C@H]2CCCN(C)C2)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1 ; O=C(O)C(F)(F)F]

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 33.5 mg (0.29 mmol) of (3S)-1-methylpiperidin-3-amine in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 99 mg (69% of theory, 92% purity) of the title compound were obtained.

LC-MS (Method 1): Rₜ=0.81 min; MS (ESIneg): m/z=778 [M−H-TFA]⁻.

### Example 130A

### tert-Butyl [(trans-4-{[(2S)-1-({4-[3-(difluoromethyl)-1H-1,2,4-triazol-5-yl]phenyl}amino)-3-(2′-methyl-4′-{[(3R)-2-oxopiperidin-3-yl]carbamoyl}biphenyl-4-yl)-1-oxopropan-2-yl]carbamoyl}-cyclohexyl)methyl]carbamate

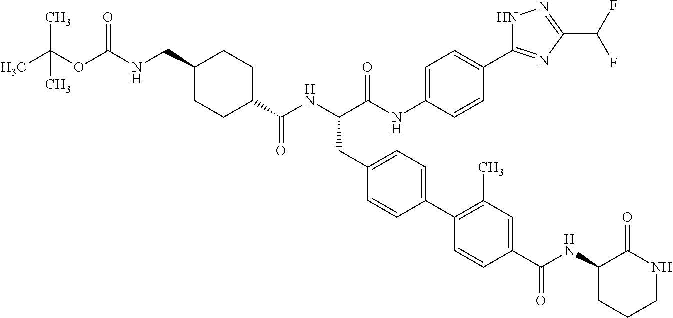

A solution of 71 mg (0.1 mmol) of 4′-[(2S)-2-{[(trans-4-{[(tert-butoxycarbonyl)amino]-methyl}cyclohexyl)carbonyl]amino}-3-oxo-3-({4-[3-(difluoromethyl)-1H-1,2,4-triazol-5-yl]phenyl}-amino)-3-oxopropyl]-2-methylbiphenyl-4-carboxylic acid and 22.2 mg (0.19 mmol) of (3R)-3-aminopiperidin-2-one in 1 ml of dimethylformamide was admixed with 51 μl (0.29 mmol) of N,N-diisopropylethylamine and 55.4 mg (0.15 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 51 mg (48% of theory, 87% purity) of the title compound were obtained.

LC-MS (Method 1): Rₜ=0.99 min; MS (ESIneg): m/z=827.4 [M−H]⁻.

### Example 131A

### tert-Butyl (3R)-3-[({4′-[(2S)-2-{[(trans-4-{[(tert-butoxycarbonyl)amino]methyl}cyclohexyl)-carbonyl]amino}-3-oxo-3-({4-[3-(pentafluoroethyl)-1H-1,2,4-triazol-5-yl]phenyl}amino)propyl]-2-methylbiphenyl-4-yl}carbonyl)amino]pyrrolidine-1-carboxylate

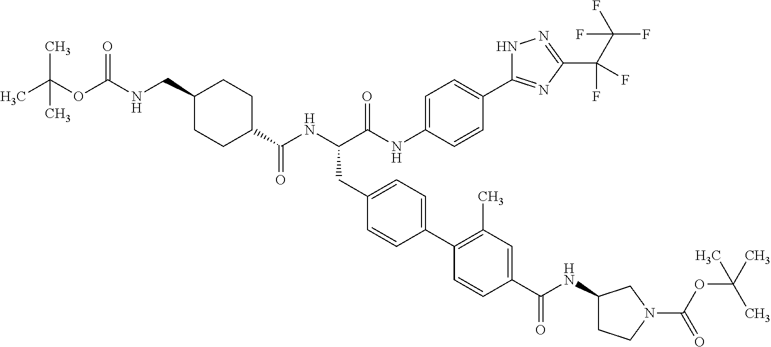

A solution of 75 mg (0.09 mmol) of 4′-[(2S)-2-{[(trans-4-{[(tert-butoxycarbonyl)amino]-methyl}cyclohexyl)carbonyl]amino}-3-oxo-3-({4-[3-(pentafluoroethyl)-1H-1,2,4-triazol-5-yl]-phenyl}amino)propyl]-2-methylbiphenyl-4-carboxylic acid and 35 mg (0.19 mmol) of tert-butyl (3R)-3-aminopyrrolidine-1-carboxylate in 1 ml of dimethylformamide was admixed with 49 μl (0.28 mmol) of N,N-diisopropylethylamine and 53.5 mg (0.14 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and acetonitrile and separated by means of preparative HPLC (eluent: acetonitrile/water gradient, 0.01% trifluoroacetic acid). The product-containing fractions were combined and concentrated on a rotary evaporator. The residue was dried under high vacuum. 76 mg (73% of theory) of the title compound were obtained.

LC-MS (Method 1): Rₜ=1.26 min; MS (ESIneg): m/z=967 [M−H]⁻.

### Example 132A

### tert-Butyl [(trans-4-{[(2S)-3-{2′-methyl-4′-[(1-methylpiperidin-4-yl)carbamoyl]biphenyl-4-yl}-1-oxo-1-{[2-(trifluoromethyl)-1H-benzimidazol-5-yl]amino}propan-2-yl]carbamoyl}cyclohexyl)-methyl]carbamate trifluoroacetate

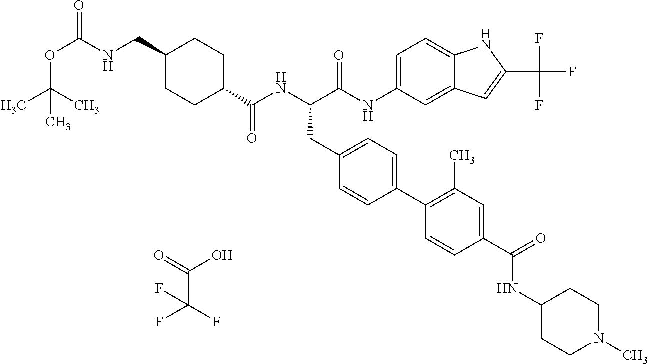 [SMILES: Cc1cc(C(=O)NC2CCN(C)CC2)ccc1-c1ccc(C[C@H](NC(=O)[C@H]2CC[C@H](CNC(=O)OC(C)(C)C)CC2)C(=O)Nc2ccc3[nH]c(C(F)(F)F)cc3c2)cc1 ; O=C(O)C(F)(F)F]

A solution of 100 mg (0.14 mmol) of 4′-[(2S)-2-{[(trans-4-{[(tert-butoxycarbonyl)amino]-methyl}cyclohexyl)carbonyl]amino}-3-oxo-3-{[2-(trifluoromethyl)-1H-benzimidazol-5-yl]amino}-propyl]-2-methylbiphenyl-4-carboxylic acid and 22.2 mg (0.19 mmol) of 1-methylpiperidin-4-amine in 1 ml of dimethylformamide was admixed with 72.4 μl (0.42 mmol) of N,N-diisopropylethylamine and 121.4 μl (0.21 mmol) of a 50% 2,4,6-tripropyl-1,3,5,2,4,6-trioxatriphosphinane 2,4,6-trioxide solution in dimethylformamide and stirred at RT for 16 h. The contents of the flask were separated by means of preparative HPLC (eluent: acetonitrile/water gradient, 0.01% trifluoroacetic acid). The product-containing fractions were combined and concentrated on a rotary evaporator. The residue was dried under high vacuum. 82 mg (69% of theory) of the title compound were obtained.

LC-MS (Method 1): Rₜ=0.85 min; MS (ESIneg): m/z=818 [M−H-TFA]⁻.

### Example 133A

### tert-Butyl [(trans-4-{[(2S)-3-(4′-{[4-(dimethylamino)cyclohexyl]carbamoyl}-2′-methylbiphenyl-4-yl)-1-oxo-1-{[2-(trifluoromethyl)-1H-benzimidazol-5-yl]amino}propan-2-yl]carbamoyl}-cyclohexyl)methyl]carbamate trifluoroacetate

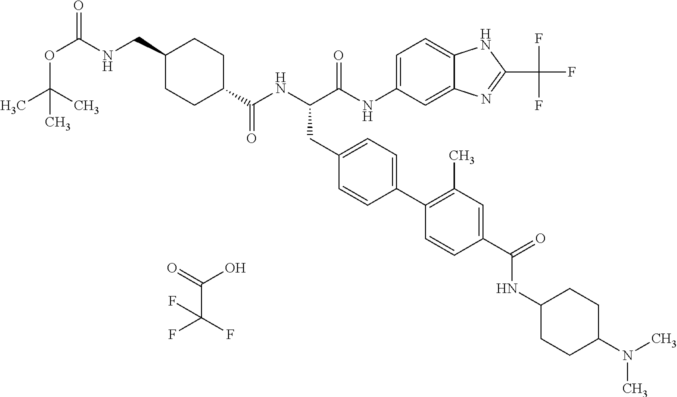 [SMILES: Cc1cc(C(=O)NC2CCC(N(C)C)CC2)ccc1-c1ccc(C[C@H](NC(=O)[C@H]2CC[C@H](CNC(=O)OC(C)(C)C)CC2)C(=O)Nc2ccc3[nH]c(C(F)(F)F)nc3c2)cc1 ; O=C(O)C(F)(F)F]

A solution of 100 mg (0.14 mmol) of 4′-[(2S)-2-{[(trans-4-{[(tert-butoxycarbonyl)amino]-methyl}cyclohexyl)carbonyl]amino}-3-oxo-3-{[2-(trifluoromethyl)-1H-benzimidazol-5-yl]amino}-propyl]-2-methylbiphenyl-4-carboxylic acid and 27.6 mg (0.19 mmol) of N,N-dimethylcyclohexane-1,4-diamine in 1 ml of dimethylformamide was admixed with 72.4 μl (0.42 mmol) of N,N-diisopropylethylamine and 121.4 μl (0.21 mmol) of a 50% 2,4,6-tripropyl-1,3,5,2,4,6-trioxatriphosphinane 2,4,6-trioxide solution in dimethylformamide and stirred at RT for 16 h. The contents of the flask were separated by means of preparative HPLC (eluent: acetonitrile/water gradient, 0.01% trifluoroacetic acid). The product-containing fractions were combined and concentrated on a rotary evaporator. The residue was dried under high vacuum. 74 mg (63% of theory) of the title compound were obtained.

LC-MS (Method 1): Rₜ=0.86 min; MS (ESIneg): m/z=846 [M−H-TFA]⁻.

### Example 134A

### tert-Butyl [(trans-4-{[(2S)-3-(4′-{[4-(dimethylamino)cyclohexyl]carbamoyl}-2′-methylbiphenyl-4-yl)-1-oxo-1-{[2-(pentafluoroethyl)-1H-benzimidazol-5-yl]amino}propan-2-yl]carbamoyl}-cyclohexyl)methyl]carbamate

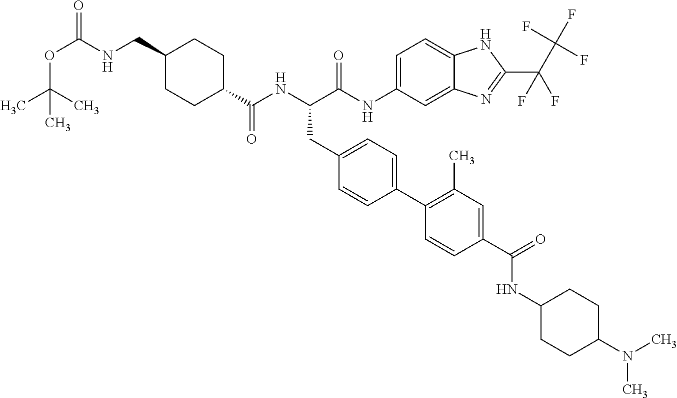 [SMILES: Cc1cc(C(=O)NC2CCC(N(C)C)CC2)ccc1-c1ccc(C[C@H](NC(=O)[C@H]2CC[C@H](CNC(=O)OC(C)(C)C)CC2)C(=O)Nc2ccc3[nH]c(C(F)(F)C(F)(F)F)nc3c2)cc1]

A solution of 100 mg (0.13 mmol) of 4′-[(2S)-2-{[(trans-4-{[(tert-butoxycarbonyl)amino]-methyl}cyclohexyl)carbonyl]amino}-3-oxo-3-{[2-(pentafluoroethyl)-1H-benzimidazol-5-yl]amino}-propyl]-2-methylbiphenyl-4-carboxylic acid and 25.8 mg (0.18 mmol) of N,N-dimethylcyclohexane-1,4-diamine in 1 ml of dimethylformamide was admixed with 67.7 μl (0.39 mmol) of N,N-diisopropylethylamine and 113.5 μl (0.19 mmol) of a 50% 2,4,6-tripropyl-1,3,5,2,4,6-trioxatriphosphinane 2,4,6-trioxide solution in dimethylformamide and stirred at RT for 16 h. The contents of the flask were diluted with water and the solid formed was filtered off and washed with water. The crystals were dried under high vacuum. 111 mg (86% of theory, 89%/0 purity) of the title compound were obtained.

LC-MS (Method 1): Rₜ=0.92 min; MS (ESIneg): m/z=896 [M−H]⁻.

### Example 135A

### tert-Butyl [(trans-4-{[(2S)-3-[4′-({trans-4-{[(tert-butoxycarbonyl)amino]cyclohexyl}carbamoyl)-2′-methylbiphenyl-4-yl]-1-oxo-1-[2-(pentafluoroethyl)-1H-benzimidazol-5-yl]amino}propan-2-yl]carbamoyl}cyclohexyl)methyl]carbamate

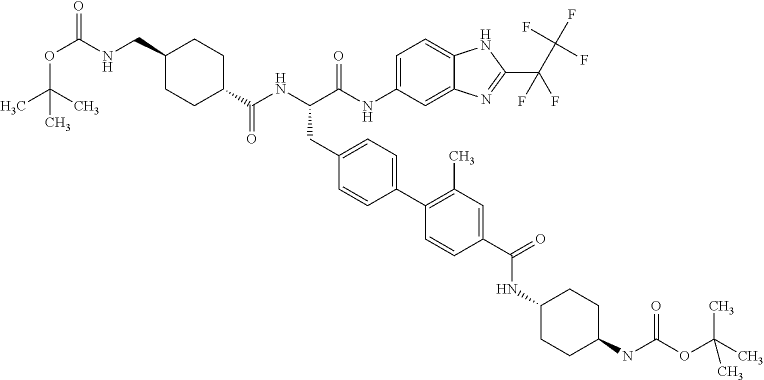 [SMILES: Cc1cc(C(=O)N[C@H]2CC[C@H](NC(=O)OC(C)(C)C)CC2)ccc1-c1ccc(C[C@H](NC(=O)[C@H]2CC[C@H](CNC(=O)OC(C)(C)C)CC2)C(=O)Nc2ccc3[nH]c(C(F)(F)C(F)(F)F)nc3c2)cc1]

A solution of 100 mg (0.13 mmol) of 4′-[(2S)-2-{[(trans-4-{[(tert-butoxycarbonyl)amino]-methyl}cyclohexyl)carbonyl]amino}-3-oxo-3-{[2-(pentafluoroethyl)-1H-benzimidazol-5-yl]amino}-propyl]-2-methylbiphenyl-4-carboxylic acid and 38.9 mg (0.18 mmol) of tert-butyl (trans-4-aminocyclohexyl)carbamate in 1 ml of dimethylformamide was admixed with 67.7 μl (0.39 mmol) of N,N-diisopropylethylamine and 113.5 μl (0.19 mmol) of a 50% 2,4,6-tripropyl-1,3,5,2,4,6-trioxatriphosphinane 2,4,6-trioxide solution in dimethylformamide and stirred at RT for 16 h. The contents of the flask were partitioned between 2 ml of 2M sodium hydroxide solution and ethyl acetate. The organic phase was washed with saturated aqueous sodium chloride solution and dried using EXTRELUT NT3 cartridges (Merck KGaA). The filtrate was concentrated under reduced pressure and dried under high vacuum. 115 mg (65% of theory, 71% purity) of the title compound were obtained.

LC-MS (Method 1): Rₜ=1.23 min; MS (ESIneg): m/z=968 [M−H]⁻.

### Example 136A

### tert-Butyl (3R)-3-[({4′-[(2S)-2-{[(trans-4-{[(tert-butoxycarbonyl)amino]methyl}cyclohexyl)-carbonyl]amino}-3-oxo-3-{[2-(pentafluoroethyl)-1H-benzimidazol-5-yl]amino}propyl]-2-methylbiphenyl-4-yl}carbonyl)amino]pyrrolidine-1-carboxylate

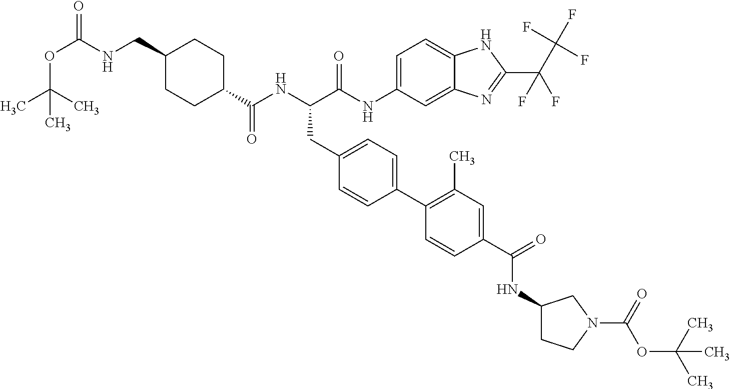 [SMILES: Cc1cc(C(=O)N[C@@H]2CCN(C(=O)OC(C)(C)C)C2)ccc1-c1ccc(C[C@H](NC(=O)[C@H]2CC[C@H](CNC(=O)OC(C)(C)C)CC2)C(=O)Nc2ccc3[nH]c(C(F)(F)C(F)(F)F)nc3c2)cc1]

A solution of 100 mg (0.13 mmol) of 4′-[(2S)-2-{[(trans-4-{[(tert-butoxycarbonyl)amino]-methyl}cyclohexyl)carbonyl]amino}-3-oxo-3-{[2-(pentafluoroethyl)-1H-benzimidazol-5-yl]amino}-propyl]-2-methylbiphenyl-4-carboxylic acid and 33.8 mg (0.18 mmol) of tert-butyl (3R)-3-aminopyrrolidine-1-carboxylate in 1 ml of dimethylformamide was admixed with 67.7 μl (0.39 mmol) of N,N-diisopropylethylamine and 113.5 μl (0.19 mmol) of a 500 2,4,6-tripropyl-1,3,5,2,4,6-trioxatriphosphinane 2,4,6-trioxide solution in dimethylformamide and stirred at RT for 16 h. The contents of the flask were partitioned between 2 ml of 2M sodium hydroxide solution and ethyl acetate. The organic phase was washed with saturated aqueous sodium chloride solution and dried using EXTRELUT NT3 cartridges (Merck KGaA). The filtrate was concentrated under reduced pressure and dried under high vacuum. 101 mg (69% of theory, 83% purity) of the title compound were obtained.

LC-MS (Method 1): Rₜ=1.22 min; MS (ESIneg): m/z=940 [M−H]⁻.

### Example 137A

### tert-Butyl [(trans-4-{[(2S)-3-(4′-{[3-(dimethylamino)propyl]carbamoyl}-2′-methylbiphenyl-4-yl)-1-oxo-1-{[2-(pentafluoroethyl)-1H-benzimidazol-5-yl]amino}propan-2-yl]carbamoyl}cyclohexyl)-methyl]carbamate

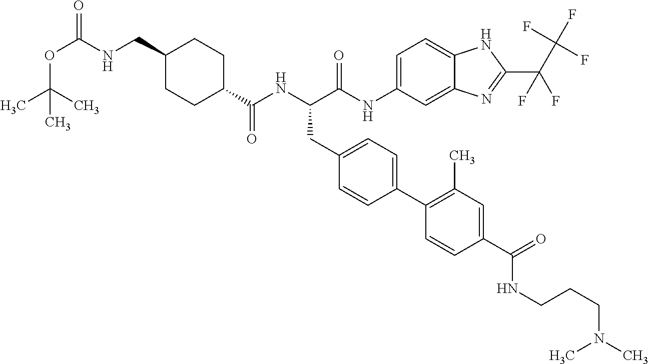 [SMILES: Cc1cc(C(=O)NCCCN(C)C)ccc1-c1ccc(C[C@H](NC(=O)[C@H]2CC[C@H](CNC(=O)OC(C)(C)C)CC2)C(=O)Nc2ccc3[nH]c(C(F)(F)C(F)(F)F)nc3c2)cc1]

A solution of 100 mg (0.13 mmol) of 4′-[(2S)-2-{[(trans-4-{[(tert-butoxycarbonyl)amino]-methyl}cyclohexyl)carbonyl]amino}-3-oxo-3-{[2-(pentafluoroethyl)-1H-benzimidazol-5-yl]amino}-propyl]-2-methylbiphenyl-4-carboxylic acid and 18.5 mg (0.18 mmol) of 3-dimethylamino-1-propylamine in 1 ml of dimethylformamide was admixed with 67.7 μl (0.39 mmol) of N,N-diisopropylethylamine and 113.5 μl (0.19 mmol) of a 50% 2,4,6-tripropyl-1,3,5,2,4,6-trioxatriphosphinane 2,4,6-trioxide solution in dimethylformamide and stirred at RT for 16 h. The contents of the flask were partitioned between 2 ml of 2M sodium hydroxide solution and ethyl acetate. The organic phase was washed with saturated aqueous sodium chloride solution and dried using EXTRELUT NT3 cartridges (Merck KGaA). The filtrate was concentrated under reduced pressure and dried under high vacuum. 45 mg (41% of theory, 62% purity) of the title compound were obtained.

LC-MS (Method 1): Rₜ=0.89 min; MS (ESIneg): m/z=856 [M−H]⁻.

### Example 138A

### tert-Butyl [(trans-4-{[(2S)-3-{2′-methyl-4′-[(1-methylpiperidin-4-yl)carbamoyl]biphenyl-4-yl}-1-oxo-1-{[2-(pentafluoroethyl)-1H-benzimidazol-5-yl]amino}propan-2-yl]carbamoyl}cyclohexyl)-methyl]carbamate trifluoroacetate

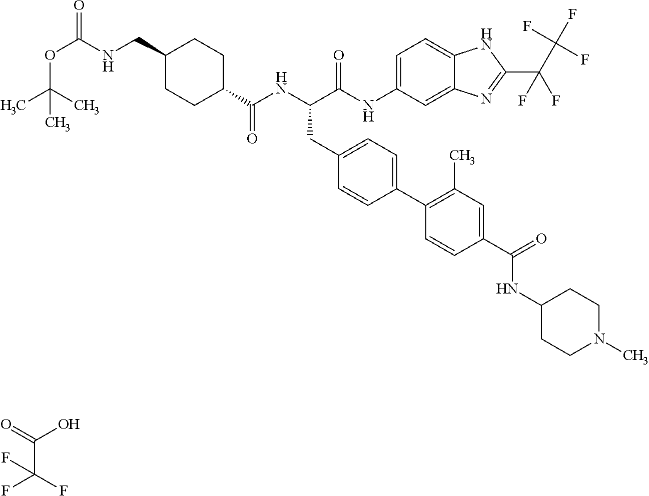 [SMILES: Cc1cc(C(=O)NC2CCN(C)CC2)ccc1-c1ccc(C[C@H](NC(=O)[C@H]2CC[C@H](CNC(=O)OC(C)(C)C)CC2)C(=O)Nc2ccc3[nH]c(C(F)(F)C(F)(F)F)nc3c2)cc1 ; O=C(O)C(F)(F)F]

A solution of 78.7 mg (0.1 mmol) of 4′-[(2S)-2-{[(trans-4-{[(tert-butoxycarbonyl)amino]-methyl}cyclohexyl)carbonyl]amino}-3-oxo-3-{[2-(pentafluoroethyl)-1H-benzimidazol-5-yl]amino}-propyl]-2-methylbiphenyl-4-carboxylic acid and 16 mg (0.14 mmol) of 1-methylpiperidin-4-amine in 0.8 ml of dimethylformamide was admixed with 53.3 μl (0.3 mmol) of N,N-diisopropylethylamine and 89.3 μl (0.15 mmol) of a 50% 2,4,6-tripropyl-1,3,5,2,4,6-trioxatriphosphinane 2,4,6-trioxide solution in dimethylformamide and stirred at RT for 16 h. The contents of the flask were separated by preparative HPLC (eluent: acetonitrile/water gradient, 0.01% trifluoroacetic acid). The product-containing fractions were combined and concentrated on a rotary evaporator. The residue was dried under high vacuum. 34 mg (39% of theory) of the title compound were obtained.

LC-MS (Method 1): Rₜ=0.89 min; MS (ESIneg): m/z=868 [M−H-TFA]⁻.

### Example 139A

### tert-Butyl (3R)-3-[({4′-[(2S)-2-{[(trans-4-{[(tert-butoxycarbonyl)amino]methyl}cyclohexyl)-carbonyl]amino}-3-{[4-(3-chloro-4H-1,2,4-triazol-5-yl)phenyl]amino}-3-oxopropyl]-2-methyl-biphenyl-4-yl}carbonyl)amino]pyrrolidine-1-carboxylate

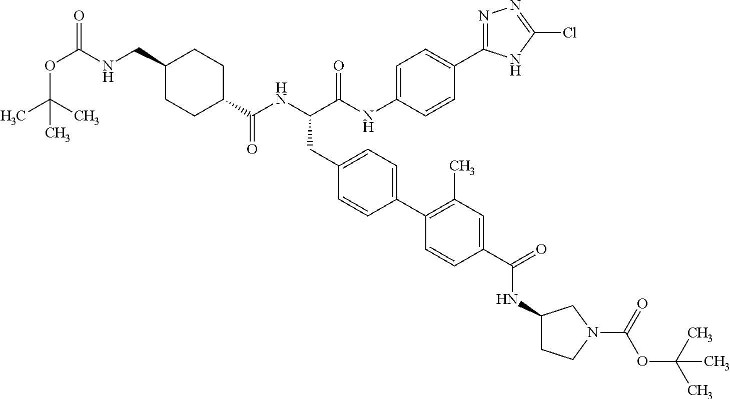 [SMILES: Cc1cc(C(=O)N[C@@H]2CCN(C(=O)OC(C)(C)C)C2)ccc1-c1ccc(C[C@H](NC(=O)[C@H]2CC[C@H](CNC(=O)OC(C)(C)C)CC2)C(=O)Nc2ccc(-c3nnc(Cl)[nH]3)cc2)cc1]

A solution of 85 mg (0.12 mmol) of 4′-[(2S)-2-{[(trans-4-{[(tert-butoxycarbonyl)amino]-methyl}cyclohexyl)carbonyl]amino}-3-{[4-(3-chloro-4H-1,2,4-triazol-5-yl)phenyl]amino}-3-oxopropyl]-2-methylbiphenyl-4-carboxylic acid and 31 mg (0.17 mmol) of tert-butyl (3R)-3-aminopyrrolidine-1-carboxylate in 1 ml of dimethylformamide was admixed with 62.1 μl (0.36 mmol) of N,N-diisopropylethylamine and 104 μl (0.18 mmol) of a 50% 2,4,6-tripropyl-1,3,5,2,4,6-trioxatriphosphinane 2,4,6-trioxide solution in dimethylformamide and stirred at RT for 16 h. The contents of the flask were partitioned between water and ethyl acetate, basified with 1M sodium hydroxide solution and extracted repeatedly with ethyl acetate. The combined organic phases were washed with saturated aqueous sodium chloride solution and dried over sodium sulphate and concentrated. The residue was separated by preparative HPLC (eluent: acetonitrile/water gradient, 0.01% trifluoroacetic acid). The product-containing fractions were combined and concentrated on a rotary evaporator. The residue was dried under high vacuum. 49 mg (33% of theory, 71% purity) of the title compound were obtained.

LC-MS (Method 1): Rₜ=1.18 min; MS (ESIneg): m/z=884 [M−H]⁻.

### Example 140A

### tert-Butyl 4-[({4′-[(2S)-2-{[(trans-4-{[(tert-butoxycarbonyl)amino]methyl}cyclohexyl)-carbonyl]amino}-3-{[4-(1H-imidazol-4-yl)phenyl]amino}-3-oxopropyl]-2-methylbiphenyl-4-yl}carbonyl)amino]piperidine-1-carboxylate

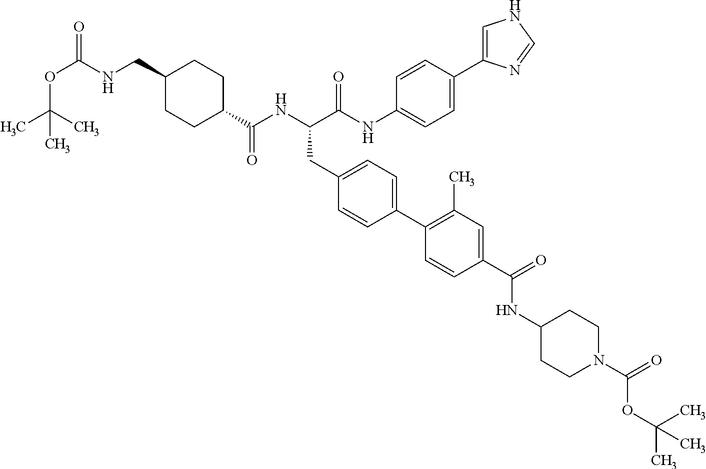 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc(-c3c[nH]cn3)cc2)cc1]

A solution of 100 mg (0.14 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 44 mg (0.28 mmol) of 4-(1H-imidazol-4-yl)aniline in 1 ml dimethylformamide was admixed with 72.5 μl (0.42 mmol) of N,N-diisopropylethylamine and 63.3 mg (0.17 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The contents of the flask were diluted with water and the precipitated solid was filtered off with suction, washed with water and dried under high vacuum. 143 mg (quant.) of the title compound were obtained.

LC-MS (Method 1): Rₜ=0.96 min; MS (ESIneg): m/z=862 [M−H]⁻.

### Example 141A

### tert-Butyl 4-[({4′-[(2S)-2-{[(trans-4-{[(tert-butoxycarbonyl)amino]methyl}cyclohexyl)-carbonyl]amino}-3-([2-(heptafluoropropyl)-1H-benzimidazol-6-yl]amino}-3-oxopropyl]-2-methylbiphenyl-4-yl}carbonyl)amino]piperidine-1-carboxylate

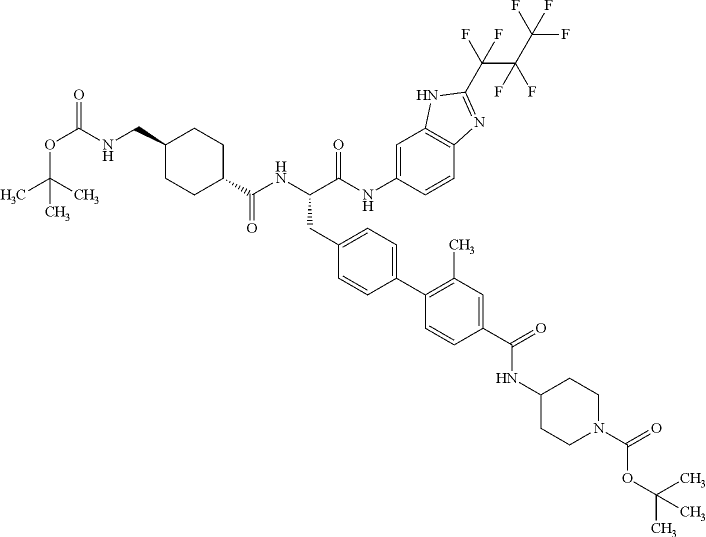 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc3nc(C(F)(F)C(F)(F)C(F)(F)F)[nH]c3c2)cc1]

A solution of 100 mg (0.14 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 93.7 mg (0.28 mmol) of 2-(heptafluoropropyl)-1H-benzimidazol-6-amine hydrochloride in 1 ml of dimethylformamide was admixed with 72.5 μl (0.42 mmol) of N,N-diisopropylethylamine and 63.3 mg (0.17 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 16 h. The contents of the flask were separated by means of preparative HPLC (eluent: acetonitrile/water gradient, 0.01% trifluoroacetic acid). The product-containing fractions were combined and concentrated on a rotary evaporator. The residue was dried under high vacuum. 74 mg (52% of theory) of the title compound were obtained.

LC-MS (Method 1): Rₜ=1.26 min; MS (ESIneg): m/z=1004 [M−H]⁻.

### Example 142A

### tert-Butyl 4-[({4′-[(2S)-2-{[(trans-4-{[(tert-butoxycarbonyl)amino]methyl}cyclohexyl)-carbonyl]amino}-3-{[2-(difluoromethyl)-1H-benzimidazol-6-yl]amino}-3-oxopropyl]-2-methylbiphenyl-4-yl}carbonyl)amino]piperidine-1-carboxylate

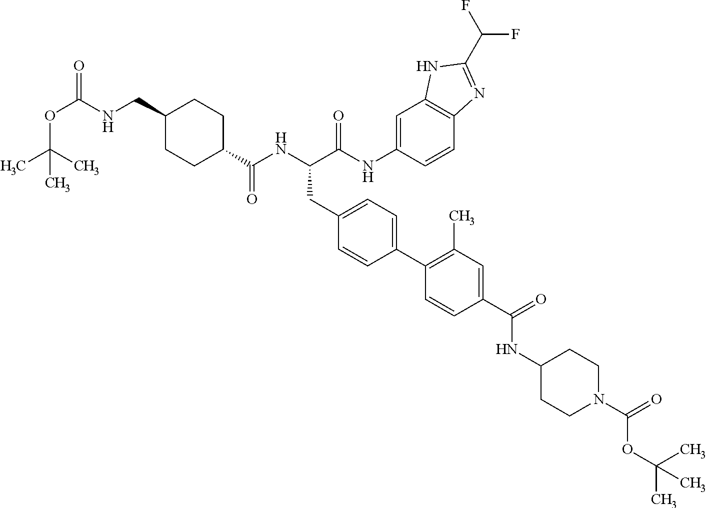 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc3nc(C(F)F)[nH]c3c2)cc1]

A solution of 113 mg (0.16 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 43.1 mg (0.24 mmol) of 2-(difluoromethyl)-1H-benzimidazol-6-amine in 1 ml of dimethylformamide was admixed with 81.9 μl (0.47 mmol) of N,N-diisopropylethylamine and 71.5 mg (0.19 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 2 h. The contents of the flask were admixed with water and the precipitated solid was filtered off and dried under high vacuum. 127 mg (91% of theory) of the title compound were obtained.

LC-MS (Method 1): Rₜ=1.16 min; MS (ESIneg): m/z=886 [M−H]⁻.

### Example 143A

### tert-Butyl [(trans-4-{[(2S)-3-(4′-{[4-(dimethylamino)cyclohexyl]carbamoyl}-2′-methylbiphenyl-4-yl)-1-({4-[3-(heptafluoropropyl)-1H-1,2,4-triazol-5-yl]phenyl}amino)-1-oxopropan-2-yl]carbamoyl}cyclohexyl)methyl]carbamate trifluoroacetate

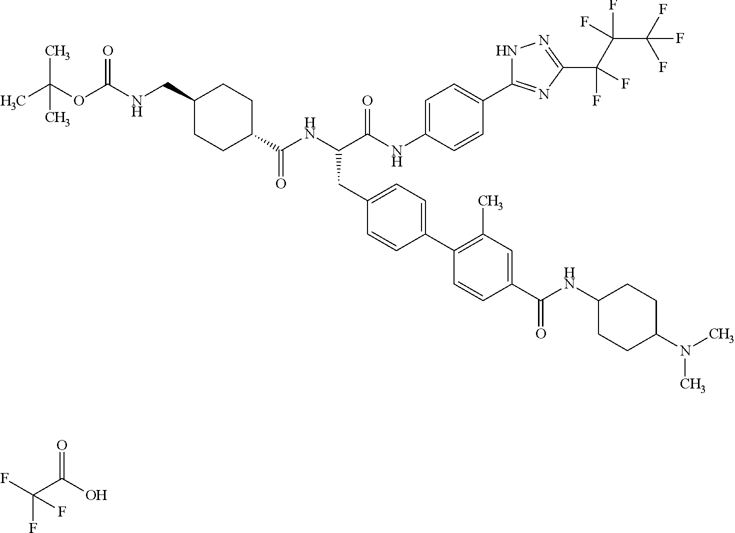

A solution of 83.5 mg (0.1 mmol) of 4′-[(2S)-2-{[(trans-4-{[(tert-butoxycarbonyl)amino]-methyl}cyclohexyl)carbonyl]amino}-3-({4-[3-(heptafluoropropyl)-1H-1,2,4-triazol-5-yl]phenyl}-amino)-3-oxopropyl]-2-methylbiphenyl-4-carboxylic acid and 28 mg (0.2 mmol) of N,N-dimethylcyclohexane-1,4-diamine in 1 ml of dimethylformamide was admixed with 51 μl (0.3 mmol) of N,N-diisopropylethylamine and 56.1 mg (0.15 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 16 h. The contents of the flask were separated by means of preparative HPLC (eluent: acetonitrile/water gradient, 0.01% trifluoroacetic acid). The product-containing fractions were combined and concentrated on a rotary evaporator. The residue was dried under high vacuum. 79 mg (55% of theory, 74% purity) of the title compound were obtained.

LC-MS (Method 1): Rₜ=1.00 min; MS (ESIneg): m/z=973 [M−H]⁻.

### Example 144A

### tert-Butyl [(trans-4-{[(2S)-1-({4-[3-(heptafluoropropyl)-1H-1,2,4-triazol-5-yl]phenyl}amino)-3-(2′-methyl-4′-[(2-oxopiperidin-3-yl)carbamoyl]biphenyl-4-yl}-1-oxopropan-2-yl]carbamoyl}-cyclohexyl)methyl]carbamate

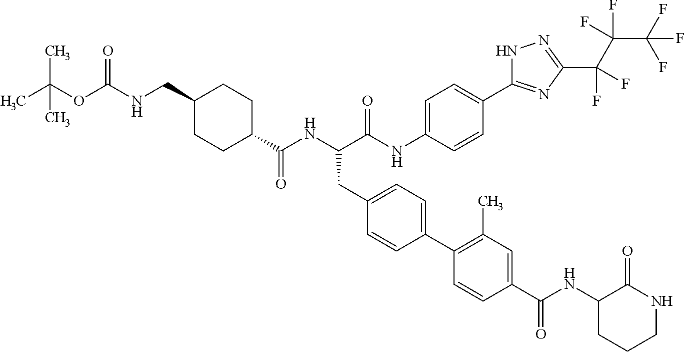

A solution of 83.5 mg (0.1 mmol) of 4′-[(2S)-2-{[(trans-4-{[(tert-butoxycarbonyl)amino]-methyl}cyclohexyl)carbonyl]amino}-3-({4-[3-(heptafluoropropyl)-1H-1,2,4-triazol-5-yl]phenyl}-amino)-3-oxopropyl]-2-methylbiphenyl-4-carboxylic acid and 22.5 mg (0.2 mmol) of 3-aminopiperidin-2-one in 1 ml of dimethylformamide was admixed with 51 μl (0.3 mmol) of N,N-diisopropylethylamine and 56.1 mg (0.15 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 16 h. The contents of the flask were separated by means of preparative HPLC (eluent: acetonitrile/water gradient, 0.01% trifluoroacetic acid). The product-containing fractions were combined and concentrated on a rotary evaporator. The residue was dried under high vacuum. 54 mg (51% of theory) of the title compound were obtained.

LC-MS (Method 1): Rₜ=1.16 min; MS (ESIneg): m/z=945 [M−H]⁻.

### Example 145A

### tert-Butyl [(trans-4-{[(2S)-1-{[3-fluoro-4-(2H-tetrazol-5-yl)phenyl]amino}-3-{4′-[(1-isopropylpiperidin-4-yl)carbamoyl]-2′-methylbiphenyl-4-yl}-1-oxopropan-2-yl]carbamoyl}cyclohexyl)-methyl]carbamate

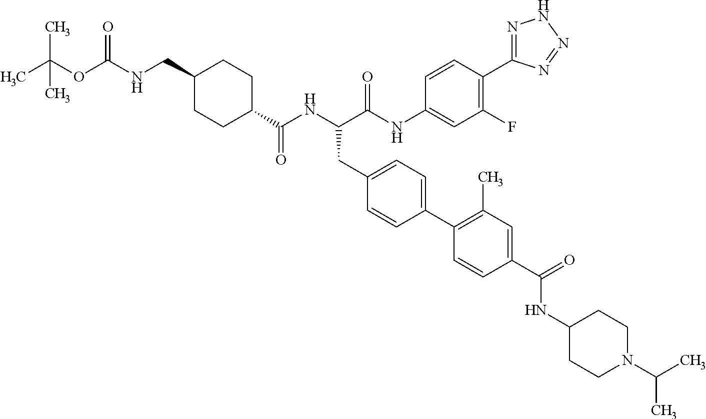

A solution of 150 mg (0.21 mmol) of 4′-[(2S)-2-{[(trans-4-{[(tert-butoxycarbonyl)amino]-methyl}cyclohexyl)carbonyl]amino}-3-{[3-fluoro-4-(2H-tetrazol-5-yl)phenyl]amino}-3-oxopropyl]-2-methylbiphenyl-4-carboxylic acid and 61 mg (0.43 mmol) of 1-isopropylpiperidin-4-amine in 2.0 ml of dimethylformamide was admixed with 0.11 ml (0.64 mmol) of N,N-diisopropylethylamine and 163 mg (0.43 mmol) of N-[(dimethylamino)(3H-[1,2,3]triazolo[4,5-b]pyridin-3-yloxy)methylidene]-N-methylmethanaminium hexafluorophosphate. The mixture was stirred at 30° C. for 48 h. The reaction mixture was separated by chromatography via HPLC (Method 11). The product-containing fractions were combined and lyophilized. This gave 45 mg (25% of theory) of the title compound.

LC-MS (Method 5): Rₜ=0.92 min; MS (ESIpos): m/z=824.5 [M+H]⁺.

### Example 146A

### tert-Butyl 5-[({4′-[(2S)-2-{[(trans-4-{[(tert-butoxycarbonyl)amino]methyl}cyclohexyl)-carbonyl]amino}-3-{[3-fluoro-4-(2H-tetrazol-5-yl)phenyl]amino}-3-oxopropyl]-2-methylbiphenyl-4-yl}carbonyl)amino]-3,3-difluoropiperidine-1-carboxylate

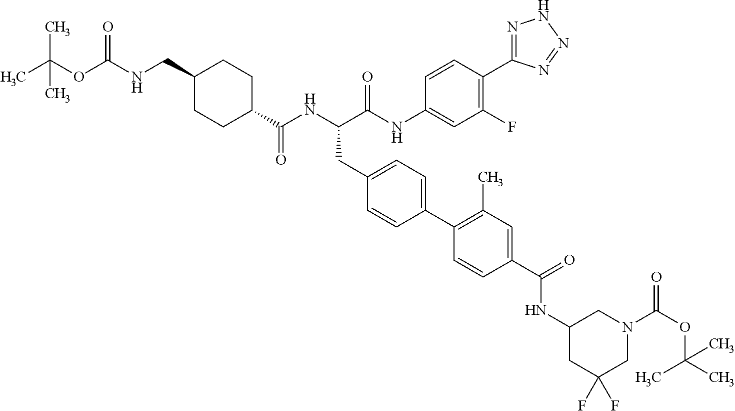 [SMILES: Cc1cc(C(=O)NC2CN(C(=O)OC(C)(C)C)CC(F)(F)C2)ccc1-c1ccc(C[C@H](NC(=O)[C@H]2CC[C@H](CNC(=O)OC(C)(C)C)CC2)C(=O)Nc2ccc(-c3nn[nH]n3)c(F)c2)cc1]

A solution of 150 mg (0.21 mmol) of 4′-[(2S)-2-{[(trans-4-{[(tert-butoxycarbonyl)amino]-methyl}cyclohexyl)carbonyl]amino}-3-{[3-fluoro-4-(2H-tetrazol-5-yl)phenyl]amino}-3-oxopropyl]-2-methylbiphenyl-4-carboxylic acid and 101 mg (0.43 mmol) of tert-butyl 5-amino-3,3-difluoropiperidine-1-carboxylate in 2.0 ml of dimethylformamide were admixed with 0.11 ml (0.64 mmol) of N,N-diisopropylethylamine and 163 mg (0.43 mmol) of N-[(dimethylamino)(3H-[1,2,3]triazolo[4,5-b]pyridin-3-yloxy)methylidene]-N-methylmethanaminium hexafluorophosphate. The mixture was stirred at 30° C. for 48 h. The reaction mixture was separated by chromatography via HPLC (Method 11). The product-containing fractions were combined and lyophilized. This gave 29 mg (15% of theory) of the title compound.

LC-MS (Method 5): Rₜ=1.0 min; MS (ESIpos): m/z=918.6 [M+H]⁺.

### Example 147A

### tert-Butyl [(trans-4-{[(2S)-1-oxo-3-(4′-{[2-(pyrrolidin-1-yl)ethyl]carbamoyl}biphenyl-4-yl)-1-{[4-(1H-tetrazol-5-yl)phenyl]amino}propan-2-yl]carbamoyl}cyclohexyl)methyl]carbamate

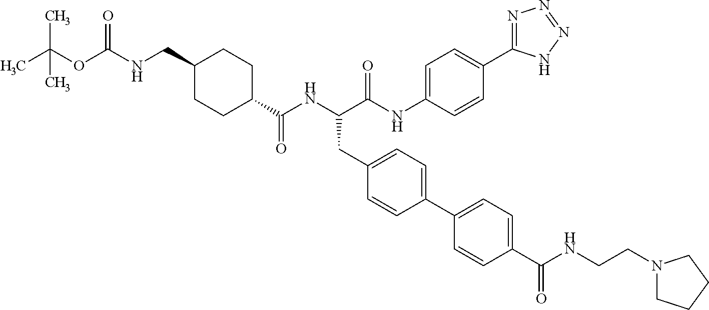

4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and (4-{[2-(pyrrolidin-1-yl)-ethyl]carbamoyl}phenyl)boronic acid hydrochloride (107 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 120 min, cooled, filtered and purified by chromatography via HPLC (Method 11). This gave 61 mg (33% of theory) of the title compound.

LC-MS (Method 5): Rₜ=0.83 min; MS (ESIpos): m/z=764.6 [M+H]⁺.

### Example 148A

### tert-Butyl [(trans-4-{[(2S)-1-{[3-fluoro-4-(2H-tetrazol-5-yl)phenyl]amino}-3-{4′-[(3-hydroxycyclo-pentyl)carbamoyl]-2′-methylbiphenyl-4-yl}-1-oxopropan-2-yl]carbamoyl}cyclohexyl)methyl]-carbamate

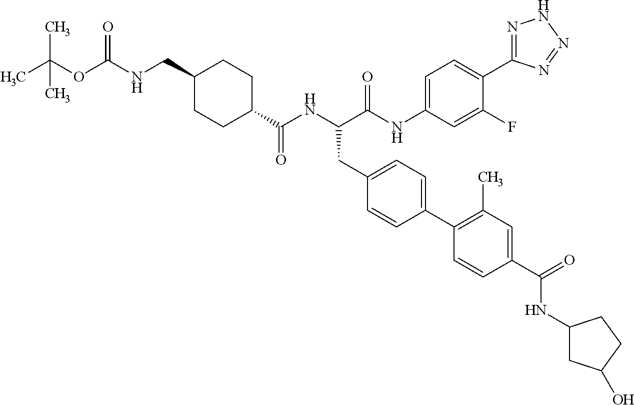

A solution of 150 mg (0.21 mmol) of 4′-[(2S)-2-{[(trans-4-{[(tert-butoxycarbonyl)amino]-methyl}cyclohexyl)carbonyl]amino}-3-{[3-fluoro-4-(2H-tetrazol-5-yl)phenyl]amino}-3-oxopropyl]-2-methylbiphenyl-4-carboxylic acid and 59 mg (0.43 mmol) of 3-aminocyclopentanol hydrochloride in 2.0 ml of dimethylformamide were admixed with 0.11 ml (0.64 mmol) of N,N-diisopropylethylamine and 163 mg (0.43 mmol) of N-[(dimethylamino)(3H-[1,2,3]triazolo[4,5-b]pyridin-3-yloxy)methylidene]-N-methylmethanaminium hexafluorophosphate. The mixture was stirred at 30° C. for 48 h. The reaction mixture was separated by chromatography via HPLC (Method 11). The product-containing fractions were combined and lyophilized. This gave 29 mg (15% of theory) of the title compound.

LC-MS (Method 5): Rₜ=0.82 min; MS (ESIpos): m/z=783.5 [M+H]⁺.

### Example 149A

### tert-Butyl [(trans-4-{[(2S)-1-[(3-chloro-1H-indazol-6-yl)amino]-3-(2′-methyl-4′-{[(3S)-2-oxopiperidin-3-yl]carbamoyl}biphenyl-4-yl)-1-oxopropan-2-yl]carbamoyl}cyclohexyl)methyl]-carbamate

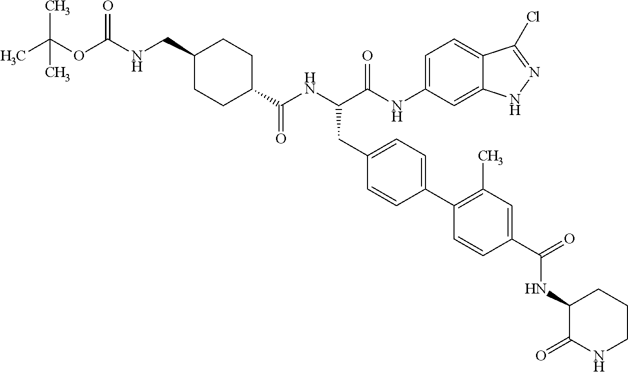

A solution of 150 mg (0.21 mmol) of 4′-[(2S)-2-{[(trans-4-{[(tert-butoxycarbonyl)amino]-methyl}cyclohexyl)carbonyl]amino}-3-[(3-chloro-1H-indazol-6-yl)amino]-3-oxopropyl]-2-methylbiphenyl-4-carboxylic acid and 50 mg (0.43 mmol) of (3S)-3-aminopiperidin-2-one in 2.0 ml of dimethylformamide were admixed with 0.11 ml (0.64 mmol) of N,N-diisopropylethylamine and 163 mg (0.43 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 24 h. The reaction mixture was separated by chromatography via HPLC (Method 11). The product-containing fractions were combined and lyophilized. This gave 14 mg (8% of theory) of the title compound.

LC-MS (Method 5): Rₜ=1.17 min; MS (ESIpos): m/z=784.6 [M+H]⁺.

### Example 150A

### tert-Butyl [(trans-4-{[(2S)-1-[(3-chloro-1H-indazol-6-yl)amino]-3-(4′-{[4-(diethylamino)-cyclohexyl]carbamoyl}-2′-methylbiphenyl-4-yl)-1-oxopropan-2-yl]carbamoyl}cyclohexyl)-methyl]carbamate

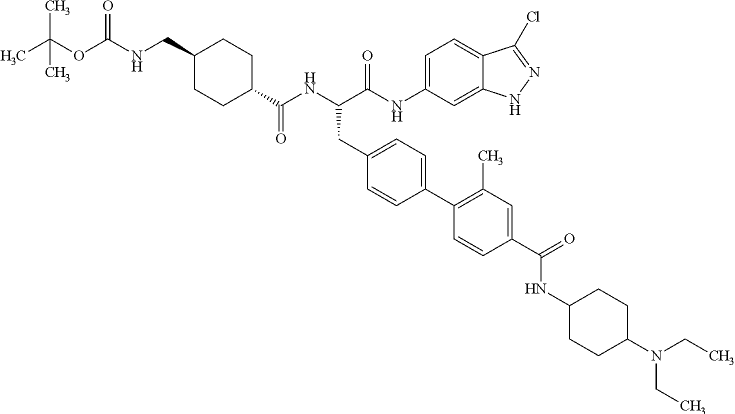

A solution of 150 mg (0.21 mmol) of 4′-[(2S)-2-{[(trans-4-{[(tert-butoxycarbonyl)amino]-methyl}cyclohexyl)carbonyl]amino}-3-[(3-chloro-1H-indazol-6-yl)amino]-3-oxopropyl]-2-methylbiphenyl-4-carboxylic acid and 74 mg (0.43 mmol) of N,N-diethylcyclohexane-1,4-diamine in 2.0 ml of dimethylformamide were admixed with 0.11 ml (0.64 mmol) of N,N-diisopropylethylamine and 163 mg (0.43 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 24 h. The reaction mixture was separated by chromatography via HPLC (Method 11). The product-containing fractions were combined and lyophilized. This gave 14 mg (8% of theory) of the title compound.

LC-MS (Method 5): R=1.51 min; MS (ESIpos): m/z=840.6 [M+H]⁺.

### Example 151A

### N-alpha-[(trans-4-{[(tert-Butoxycarbonyl)amino]methyl})cyclohexyl)carbonyl]-4-iodo-N-(3-oxo-2,3-dihydro-1H-indazol-6-yl)-L-phenylalaninamide

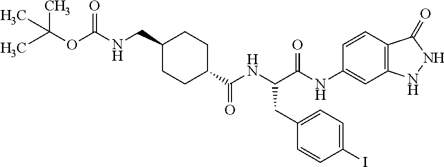

N-[(trans-4-{[(tert-Butoxycarbonyl)amino]methyl}cyclohexyl)carbonyl]-4-iodo-L-phenylalanine (1.91 g, 3.6 mmol), 6-amino-1,2-dihydro-3H-indazol-3-one (0.55 g, 3.60 mmol) and N,N-diisopropylamine (1.9 ml, 10.8 mmol) were suspended in 23 ml of ethyl acetate and admixed with 2,4,6-tripropyl-1,3,5,2,4,6-trioxatriphosphinane 2,4,6-trioxide (50% in ethyl acetate, 5.73 g, 9.0 mmol). This was followed by refluxing for 3 h, addition of further 6-amino-1,2-dihydro-3H-indazol-3-one (0.14 g, 0.90 mmol), N,N-diisopropylamine (0.47 ml, 2.70 mmol) and 2,4,6-tripropyl-1,3,5,2,4,6-trioxatriphosphinane 2,4,6-trioxide (50% in ethyl acetate, 1.43 g, 2.25 mmol) and refluxing once again for 3 h. The reaction mixture was admixed with water, the phases were separated and the aqueous phase was extracted three times with ethyl acetate. The solid precipitated in the two phases was filtered off with suction and dried under high vacuum. This gave 1.35 g (57% of theory) of the title compound.

¹H NMR (300 MHz, DMSO-d₆): δ=0.66-0.91 (m, 2H), 1.01-1.25 (m, 4H), 1.33 (s, 9H), 1.44-1.54 (m, 1H), 1.62 (m, 3H), 1.98-2.10 (m, 1H), 2.66-2.80 (m, 3H), 2.92 (dd, 1H), 4.49-4.61 (m, 1H), 6.70-6.76 (m, 1H), 6.79 (d, 1H), 6.94 (dd, 1H), 7.05 (d, 2H), 7.38 (d, 1H), 7.59 (d, 2H), 8.00 (d, 1H), 9.91 (s, 1H), 10.46 (s, 1H), 10.51 (s, 1H).

LC-MS (Method 4): Rₜ=1.15 min; MS (ESIpos): m/z=662.1 [M+H]⁺.

### Example 152A

### tert-Butyl [(trans-4-{[(2S)-3-[4′-(cyclopropylcarbamoyl)-3′-methylbiphenyl-4-yl]-1-oxo-1-{[4-(1H-tetrazol-5-yl)phenyl]amino}propan-2-yl]carbamoyl}cyclohexyl)methyl]carbamate

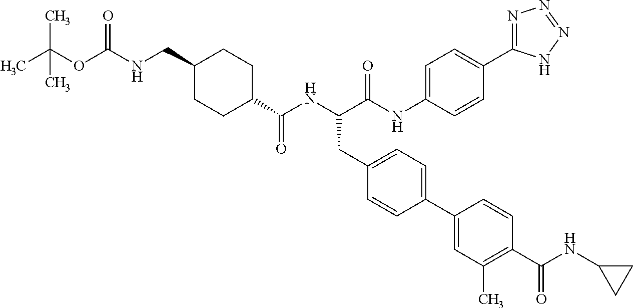

4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (150 mg, 0.24 mmol) and [4-(cyclopropyl-carbamoyl)-3-methylphenyl]boronic acid (79 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 120 min, cooled, filtered and purified by chromatography via HPLC (Method 11). This gave 88 mg (51% of theory) of the title compound.

LC-MS (Method 1): Rₜ=0.83 min; MS (ESIpos): m/z=721.5 [M+H]⁺.

### Example 153A

### tert-Butyl {[trans-4-({(2S)-1-{[3-fluoro-4-(2H-tetrazol-5-yl)phenyl]amino}-3-[4′-(isopropyl-carbamoyl)-2′-methylbiphenyl-4-yl]-1-oxopropan-2-yl}carbamoyl)cyclohexyl]methyl}carbamate

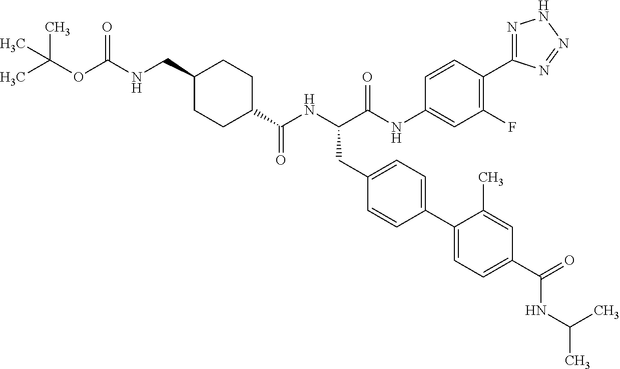

A solution of 150 mg (0.21 mmol) of 4′-[(2S)-2-{[(trans-4-{[(tert-butoxycarbonyl)amino]-methyl}cyclohexyl)carbonyl]amino}-3-{[3-fluoro-4-(2H-tetrazol-5-yl)phenyl]amino}-3-oxopropyl]-2-methylbiphenyl-4-carboxylic acid and 25 mg (0.42 mmol) of isopropylamine in 1.9 ml of dimethylformamide were admixed with 0.11 ml (0.64 mmol) of N,N-diisopropylethylamine and 163 mg (0.42 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 48 h. The reaction mixture was separated by chromatography via HPLC (Method 11). The product-containing fractions were combined and lyophilized. This gave 54 mg (34% of theory) of the title compound.

LC-MS (Method 1): Rₜ=0.89 min; MS (ESIpos): m/z=741.6 [M+H]⁺.

### Example 154A

### tert-Butyl {[trans-4-({(2S)-3-(4′-{[(3R,5S)-5-(hydroxymethyl)-2-oxopyrrolidin-3-yl]carbamoyl}-2′-methylbiphenyl-4-yl)-1-oxo-1-[(2-oxo-2,3-dihydro-1H-benzimidazol-5-yl)amino]propan-2-yl}carbamoyl)cyclohexyl]methyl}carbamate

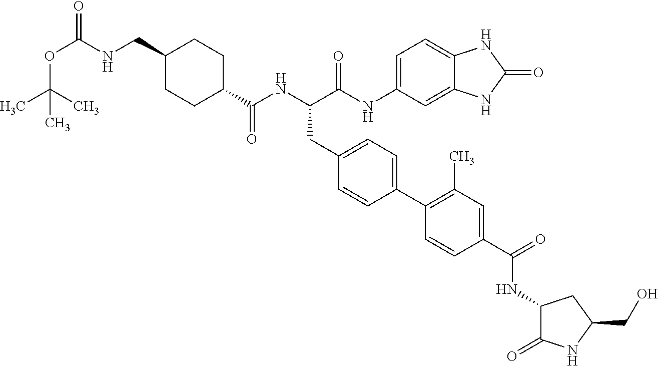 [SMILES: Cc1cc(C(=O)N[C@@H]2C[C@@H](CO)NC2=O)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

A solution of 80 mg (0.12 mmol) of 4′-{(2S)-2-{[(trans-4-{[(tert-butoxycarbonyl)amino]methyl}-cyclohexyl)carbonyl]amino}-3-oxo-3-[(2-oxo-2,3-dihydro-1H-benzimidazol-5-yl)amino]propyl}-2-methylbiphenyl-4-carboxylic acid and 28 mg (0.17 mmol) of (3R,5S)-3-amino-5-(hydroxymethyl)-pyrrolidin-2-one hydrochloride (described in: R. Goswami, M. G. Moloney, Chem. Comm. 1999, 23, 2333-2334 and E. L. Bentz, R. Goswami, M. G. Moloney, S. M. Westaway, Org. Biomol. Chem., 2005, 3, 2872-2882) in 0.9 ml of dimethylformamide was admixed with 62.1 μl (0.36 mmol) of N,N-diisopropylethylamine and 104 μl (0.18 mmol) of a 50% 2,4,6-tripropyl-1,3,5,2,4,6-trioxatriphosphinane 2,4,6-trioxide solution in dimethylformamide and stirred at RT for 16 h. The mixture was left alone for 3 days. Another 9 mg (5.7 mmol) of (3R,5S)-3-amino-5-(hydroxymethyl)pyrrolidin-2-one hydrochloride, 21 μl (0.12 mmol) of N,N-diisopropylethylamine and 51 μl (0.6 mmol) of a 50% 2,4,6-tripropyl-1,3,5,2,4,6-trioxatriphosphinane 2,4,6-trioxide solution in dimethylformamide were added and the mixture was stirred at RT for 16 h. 45.4 mg (0.12 mmol) of N-[(dimethylamino)(3H-[1,2,3]triazolo[4,5-b]pyridin-3-yloxy)methylidene]-N-methylmethanaminium hexafluorophosphate were added and the mixture was stirred at RT for 3 h. The contents of the flask were admixed with water, and the solid formed was filtered off and dried under high vacuum. 28 mg (29% of theory) of the title compound were obtained.

LC-MS (Method 1): Rₜ=0.78 min; MS (ESIpos): m/z=782 [M+H]⁺.

### Example 155A

### 2,2,3,3-Tetrafluoro-3-[5-(4-nitrophenyl)-1H-1,2,4-triazol-3-yl]propanoic acid

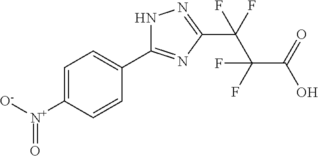 [SMILES: O=C(O)C(F)(F)C(F)(F)c1n[nH]c(-c2ccc([N+](=O)[O-])cc2)n1]

A solution of 1700 mg (9.4 mmol) of 4-nitrobenzenecarboximidohydrazide (described in: J. Liebigs Ann. Chem. 1897, 298, 51-52) in 30 ml of dichloromethane was stirred with 4870 mg (28 mmol) of 3,3,4,4-tetrafluorodihydrofuran-2,5-dione at RT for 2 min. Then 30 ml of acetonitrile were added and the mixture was stirred at RT for 16 h. Dry molecular sieve (4 Å) was added and the mixture was stirred at RT for a further 24 h. The molecular sieve was filtered off and the filtrate was concentrated. The residue was separated by means of preparative HPLC (eluent: gradient of acetonitrile/water with 0.1% trifluoroacetic acid). The product-containing fractions were combined and concentrated on a rotary evaporator. The residue was dried under high vacuum. 2434 mg (77% of theory) of the title compound were obtained. ¹H NMR (400 MHz, DMSO-d₆) δ ppm 8.28 (d, 2H), 8.44 (d, 2H), 15.64 (br. s, 1H).

LC-MS (Method 1): Rₜ=0.62 min; MS (ESIneg): m/z=333 [M−H]⁻.

### Example 156A

### 3-[5-(4-Aminophenyl)-1H-1,2,4-triazol-3-yl]-2,2,3,3-tetrafluoropropanoic acid hydrochloride

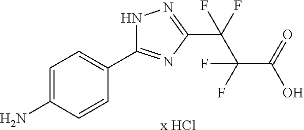 [SMILES: Nc1ccc(-c2nc(C(F)(F)C(F)(F)C(=O)O)n[nH]2)cc1 ; "x HCl"]

A solution of 2425 mg (7.3 mmol) of 2,2,3,3-tetrafluoro-3-[5-(4-nitrophenyl)-1H-1,2,4-triazol-3-yl]propanoic acid and 6549 mg (29 mmol) of tin(II) chloride dihydrate in 50 ml of ethanol was stirred at 70° C. for 1 h. The reaction solution was poured into ice-water and adjusted to pH 8 with sodium hydrogencarbonate. The mixture was diluted with water and extracted three times with ethyl acetate. The organic phases were combined, washed with saturated aqueous sodium chloride solution, dried over sodium sulphate, filtered and then concentrated. The aqueous phase was admixed with 1N hydrochloric acid solution and lyophilized. The residue was stirred with acetone, filtered and dried. The operation was repeated once again. The combined residues were dissolved in dioxane and admixed with 3.6 ml of hydrogen chloride in dioxane (4N), concentrated again and dried. 2547 mg (91% of theory) of the title compound were obtained.

¹H NMR (400 MHz, DMSO-d₆) δ ppm 6.83 (d, 2H), 7.77 (d, 2H), 14.81 (br. s, 1H).

LC-MS (Method 1): Rₜ=0.27 min; MS (ESIneg): m/z=303 [M−H—HCl]⁻.

### Example 157A

### 3-[5-(4-{[(2S)-2-{[(trans-4-{[(tert-Butoxycarbonyl)amino]methyl}cyclohexyl)carbonyl]amino}-3-(4′-{[1-(tert-butoxycarbonyl)piperidin-4-yl]carbamoyl}-2′-methylbiphenyl-4-yl)propanoyl]amino}-phenyl)-1H-1,2,4-triazol-3-yl]-2,2,3,3-tetrafluoropropanoic acid

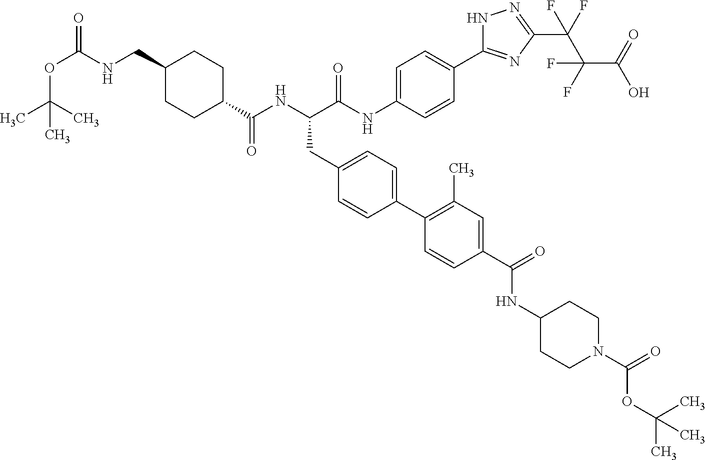 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc(-c3nc(C(F)(F)C(F)(F)C(=O)O)n[nH]3)cc2)cc1]

A solution of 150 mg (0.21 mmol) of (2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoic acid and 157 mg (0.42 mmol) of 3-[5-(4-aminophenyl)-1H-1,2,4-triazol-3-yl]-2,2,3,3-tetrafluoropropanoic acid hydrochloride in 3.5 ml dimethylformamide was admixed with 145 μl (0.83 mmol) of N,N-diisopropylethylamine and 95 mg (0.25 mmol) of N-[(dimethylamino)(3H-[1,2,3]triazolo[4,5-b]pyridin-3-yloxy)methylidene]-N-methylmethanaminium hexafluorophosphate and stirred at RT for 3 days. The reaction solution was separated by means of preparative HPLC (eluent: gradient of acetonitrile/water with 0.1% trifluoroacetic acid). The product-containing fractions were combined and concentrated on a rotary evaporator. The residue was dried under high vacuum. 64 mg (28% of theory, 92% purity) of the title compound were obtained.

LC-MS (Method 1): Rₜ=1.13 min; MS (ESIpos): m/z=1007 [M+H]⁺.

### Example 158A

### 4-Bromo-N-alpha-[(trans-4-{[(tert-butoxycarbonyl)amino]methyl}cyclohexyl)carbonyl]-N-(7-fluoro-2-oxo-2,3-dihydro-1,3-benzoxazol-5-yl)-L-phenylalaninamide

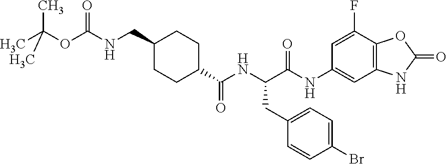

A solution of 4-bromo-N-[(trans-4-{[(tert-butoxycarbonyl)amino]methyl}-cyclohexyl)-carbonyl]-L-phenylalanine (2.5 g, 5.14 mmol) and 5-amino-7-fluoro-1,3-benzoxazol-2(3H)-one (1.0 g, 5.65 mmol) in ethyl acetate (12 ml) was admixed with N,N-diisopropylethylamine (2.68 ml, 15 mmol) and 2,4,6-tripropyl-1,3,5,2,4,6-trioxatriphosphinane 2,4,6-trioxide solution (50% in ethyl acetate, 8.2 g, 13 mmol) and then stirred under reflux for 9 h. The reaction mixture was admixed with water, the phases were separated and the aqueous phase was extracted three times with ethyl acetate. The combined organic phases were washed with saturated aqueous ammonium hydrogencarbonate solution and saturated aqueous sodium chloride solution, dried over sodium sulphate and concentrated. The crude product was purified by chromatography via HPLC (Method 11). This gave 942 mg (27% of theory) of the title compound.

¹H NMR (300 MHz, DMSO-d₆): δ=ppm 0.71-0.91 (m, 2H), 1.05-1.30 (m, 4H), 1.36 (s, 9H), 1.48-1.58 (m, 1H), 1.59-1.71 (m, 3H), 2.01-2.12 (m, 1H), 2.69-2.77 (m, 2H), 2.82 (dd, 1H), 2.97 (dd, 1H), 4.49-4.63 (m, 1H), 6.71-6.83 (m, 1H), 7.16-7.30 (m, 4H), 7.46 (d, 2H), 8.12 (d, 1H), 10.28 (s, 1H).

LC-MS (Method 5): Rₜ=0.83 min; MS (ESIneg): m/z=633.2 [M−H]⁻.

### Example 159A

### 3-Methyl-N-[(3S)-2-oxopyrrolidin-3-yl]-4-(4,4,5,5-tetramethyl-1,3,2-dioxaborolan-2-yl)benzamide

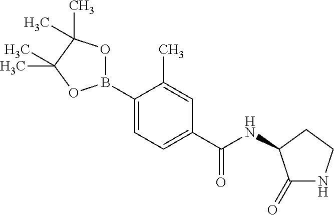 [SMILES: Cc1cc(C(=O)N[C@H]2CCNC2=O)ccc1B1OC(C)(C)C(C)(C)O1]

3-Methyl-4-(4,4,5,5-tetramethyl-1,3,2-dioxaborolan-2-yl)benzoic acid (500 mg, 1.9 mmol) and (3S)-3-aminopyrrolidin-2-one (267 mg, 2.7 mmol) were dissolved in dimethylformamide (17 ml) and admixed with diisopropylethylamine (1 ml, 5.7 mmol) and O-(7-azabenzotriazol-1-yl)-N,N,N′N′-tetramethyl-uronium hexafluorophosphate (1.4 g, 3.8 mmol). The reaction mixture was stirred at RT overnight and at 60° C. for 3 h and then purified by chromatography via flash chromatography (Isolera, eluent dichloromethane/methanol 95/5 to 90/10). This gave 656 mg (quant.) of the title compound.

LC-MS (Method 4): Rₜ=1.02 min; MS (ESIpos): m/z=344.2 [M+H]⁺.

### Example 160A

### tert-Butyl [(trans-4-{[(2S)-1-[(7-fluoro-2-oxo-2,3-dihydro-1,3-benzoxazol-5-yl)amino]-3-(2′-methyl-4′-{[(3S)-2-oxopyrrolidin-3-yl]carbamoyl}biphenyl-4-yl)-1-oxopropan-2-yl]carbamoyl}-cyclohexyl)methyl]carbamate

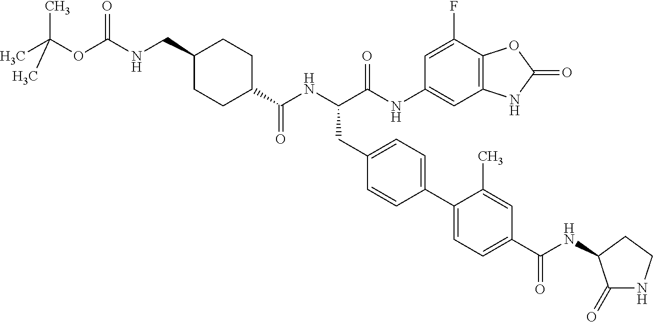

4-Bromo-N-alpha-[(trans-4-{[(tert-butoxycarbonyl)amino]methyl}cyclohexyl)carbonyl]-N-(7-fluoro-2-oxo-2,3-dihydro-1,3-benzoxazol-5-yl)-L-phenylalaninamide (100 mg, 0.15 mmol) and 3-methyl-N-[(3S)-2-oxopyrrolidin-3-yl]-4-(4,4,5,5-tetramethyl-1,3,2-dioxaborolan-2-yl)benzamide (77 mg, 0.22 mmol) were dissolved in 1.5 ml of dimethyl sulphoxide and admixed with tetrakis(triphenylphosphine)-palladium(0) (17 mg, 0.015 mmol), sodium carbonate (47 mg, 0.44 mmol) and water (0.22 ml, 12 mmol). The reaction mixture was stirred at 110° C. in a microwave (Biotage Initiator) for 90 min and then purified by chromatography via HPLC (Method 11). This gave 34 mg (30% of theory) of the title compound.

LC-MS (Method 5): R=0.79 min; MS (ESIpos): m/z=771.5 [M+H]⁺.

### Example 161A

### 3-{5-[4-({(2S)-2-{[(trans-4-{[(tert-Butoxycarbonyl)amino]methyl}cyclohexyl)carbonyl]-amino}-3-[4′-(methoxycarbonyl)-2′-methylbiphenyl-4-yl]propanoyl}amino)phenyl]-1H-1,2,4-triazol-3-yl}-2,2,3,3-tetrafluoropropanoic acid

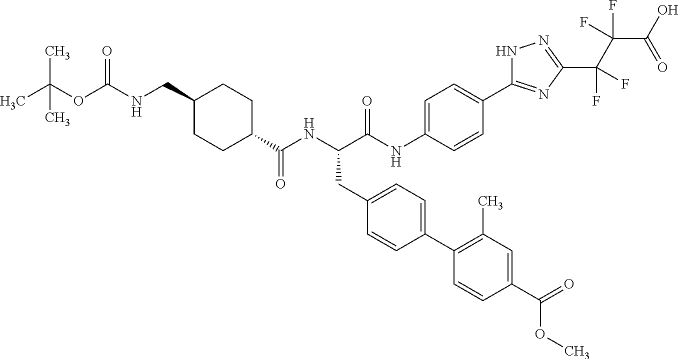 [SMILES: COC(=O)c1ccc(-c2ccc(C[C@H](NC(=O)[C@H]3CC[C@H](CNC(=O)OC(C)(C)C)CC3)C(=O)Nc3ccc(-c4nc(C(F)(F)C(F)(F)C(=O)O)n[nH]4)cc3)cc2)c(C)c1]

(2S)-2-{[(trans-4-{[(ter-Butoxycarbonyl)amino]methyl}cyclohexyl)carbonyl]amino}-3-[4′-(methoxycabonyl)-2′-methylbiphenyl-4-yl]propanoic acid (1.35 g, 2.44 mmol) and 3-[5-(4-Aminophenyl)-1H-1,2,4-triazol-3-yl]-2,2,3,3-tetrafluoropropanoic acid hydrochloride (1.84 g, 4.9 mmol) were dissolved in 20 ml of dimethylformamide and admixed with N,N-diisopropylethylamine (2.13 ml, 12.2 mmol) and N-[(dimethylamino)(3H-[1,2,3]triazolo[4,5-b]pyridin-3-yloxy)methylidene]-N-methylmethanaminium hexafluorophosphate (1.4 g, 3.7 mmol). The reaction mixture was stirred at RT overnight and concentrated. The residue was taken up in DMSO, filtered through a Millipore filter and purified by chromatography via HPLC (eluent acetonitrile/water with 0.1% trifluoroacetic acid (gradient)). This gave 690 mg (30% of theory, 90% purity) of the title compound.

LC-MS (Method 1): Rₜ=1.15 min; MS (ESIpos): m/z=839.2 [M+H]⁺.

### Example 162A

### 4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxopropyl]-2-methylbiphenyl-4-carboxylic acid

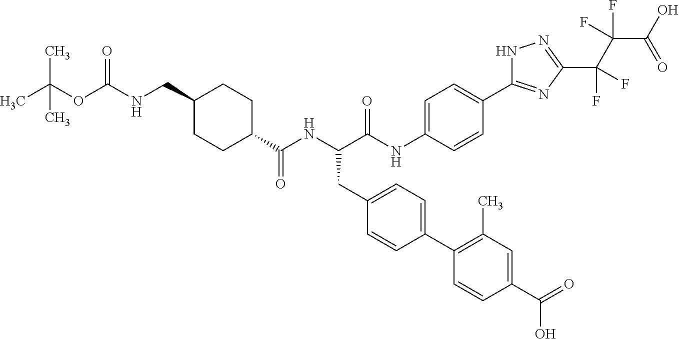

3-{5-[4-({(2S)-2-{[(trans-4-{[(tert-Butoxycarbonyl)amino]methyl}cyclohexyl)carbonyl]-amino}-3-[4′-(methoxycarbonyl)-2′-methylbiphenyl-4-yl]propanoyl}amino)phenyl]-1H-1,2,4-triazol-3-yl}-2,2,3,3-tetrafluoropropanoic acid (654 mg, 0.78 mmol) was initially charged in a solvent mixture (tetrahydrofuran/water 3:1, 12 ml) and admixed with lithium hydroxide monohydrate (327 mg, 7.8 mmol) and stirred at RT for 16 h. The reaction mixture was admixed with ethyl acetate, washed twice with 0.5N aqueous hydrogen chloride solution and once with saturated aqueous sodium chloride solution, dried over sodium sulphate and concentrated. The precipitated solid was filtered off with suction, washed twice with ethyl acetate and dried under high vacuum. This gave 633 mg (94% of theory) of the title compound.

¹H NMR (400 MHz, DMSO-d₆): δ=ppm 0.72-0.91 (m, 2H), 1.03-1.15 (m, 2H), 1.22-1.29 (m, 2H), 1.37 (s, 9H), 1.48-1.58 (m, 1H), 1.67 (m, 4H), 2.07-2.16 (m, 1H), 2.24 (s, 3H), 2.74 (m, 2H), 2.94 (dd, 1H), 3.11 (dd, 1H), 4.69-4.80 (m, 1H), 6.71-6.83 (m, 1H), 7.22-7.32 (m, 4H), 7.39 (d, 2H), 7.75-7.82 (m, 3H), 7.85 (s, 1H), 7.96 (d, 2H), 8.17 (d, 1H), 10.41 (s, 1H), 12.58-13.06 (m, 1H), 15.03 (s, 1H).

LC-MS (Method 1): Rₜ=0.96 min; MS (ESIpos): m/z=825.3 [M+H]⁺.

### Example 163A

### 3-[5-(4-{[(2S)-2-{[(trans-4-{[(tert-Butoxycarbonyl)amino]methyl}cyclohexyl)carbonyl]-amino}-3-{2′-methyl-4′-[(1-methylpiperidin-4-yl)carbamoyl]biphenyl-4-yl}propanoyl]-amino}phenyl)-1H-1,2,4-triazol-3-yl]-2,2,3,3-tetrafluoropropanoic acid

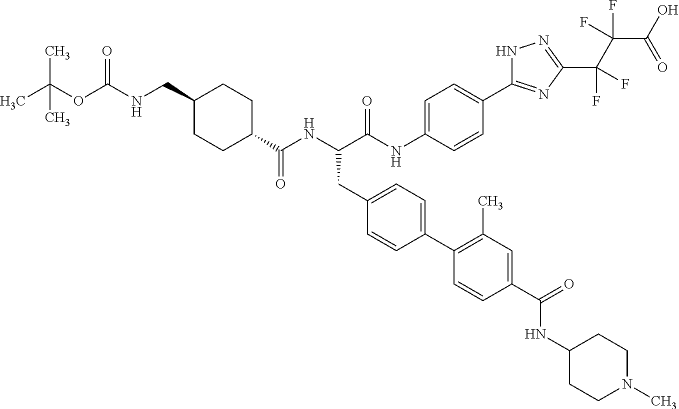

4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxo-propyl]-2-methylbiphenyl-4-carboxylic acid (80 mg, 0.1 mmol) and 1-methylpiperidin-4-amine (22 mg, 0.2 mmol) were dissolved in 1 ml of dimethylformamide and admixed with N,N-diisopropylethylamine (0.05 ml, 0.3 mmol) and N-[(dimethylamino)(3H-[1,2,3]triazolo[4,5-b]pyridin-3-yloxy)methylidene]-N-methylmethanaminium hexafluorophosphate (55 mg, 0.15 mmol) and stirred at RT for 16 h. The reaction mixture was filtered through a Millipore filter and purified via preparative HPLC (eluent: acetonitrile/water with 0.1% trifluoroacetic acid (gradient)). This gave 49 mg (55% of theory) of the title compound.

LC-MS (Method 1): Rₜ=0.82 min; MS (ESIpos): m/z=921.5 [M+H]⁺.

### Example 164A

### 3-[5-(4-{[(2S)-2-{[(trans-4-{[(tert-Butoxycarbonyl)amino]methyl}cyclohexyl)carbonyl]amino}-3-(4′-{[4-(dimethylamino)cyclohexyl]carbamoyl}-2′-methylbiphenyl-4-yl)propanoyl]amino}phenyl)-1H-1,2,4-triazol-3-yl]-2,2,3,3-tetrafluoropropanoic acid

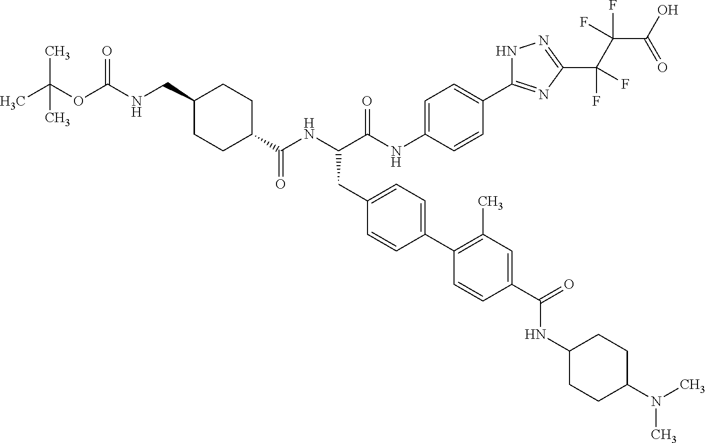

4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxo-propyl]-2-methylbiphenyl-4-carboxylic acid (80 mg, 0.1 mmol) and N,N-dimethylcyclohexane-1,4-diamine (28 mg, 0.2 mmol) were dissolved in 1 ml of dimethylformamide and admixed with N,N-diisopropylethylamine (0.05 ml, 0.3 mmol) and N-[(dimethylamino)(3H-[1,2,3]triazolo[4,5-b]pyridin-3-yloxy)methylidene]-N-methylmethanaminium hexafluorophosphate (55 mg, 0.15 mmol) and stirred at RT for 16 h. The reaction mixture was filtered through a Millipore filter and purified twice by preparative HPLC (eluent: acetonitrile/water with 0.1% trifluoroacetic acid (gradient)). This gave 11 mg (11% of theory) of the title compound.

LC-MS (Method 1): Rₜ=0.83 min; MS (ESIpos): m/z=949.5 [M+H]⁺.

### Example 165A

### 4-Bromo-N-(trans-4-hydroxycyclohexyl)-3-methylbenzamide

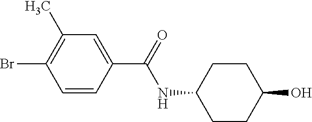 [SMILES: Cc1cc(C(=O)N[C@H]2CC[C@H](O)CC2)ccc1Br]

4-Bromo-3-methylbenzoic acid (121.0 g, 562.7 mmol) and trans-4-aminocyclohexanol (71.3 g, 618.9 mmol) were initially charged in 1.40 l of DMF and admixed with N,N-diisopropylethylamine (294.0 ml, 1688.0 mmol). Subsequently, N-[(dimethylamino)(3H-[1,2,3]triazolo[4,5-b]pyridin-3-yloxy)methylidene]-N-methylmethanaminium hexafluorophosphate (320.9 g, 844.0 mmol) were added in portions, in the course of which the reaction temperature was kept below 30° C. by cooling with ice. The reaction mixture was subsequently stirred into water, and the solid was filtered off with suction and washed with water. The still-moist filter residue was stirred in acetonitrile, filtered off with suction and washed through with acetonitrile. After air drying, this gave 157.2 g of the title compound, which was converted further without further purification.

LC-MS (Method 1): Rₜ=0.83 min; MS (ESIpos): m/z=312 [M+H]⁺.

### Example 166A

### 4-Bromo-N-(trans-4-{[tert-butyl(dimethyl)silyl]oxy}cyclohexyl)-3-methylbenzamide

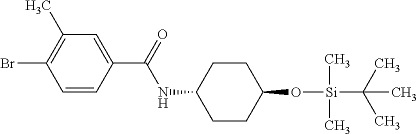 [SMILES: Cc1cc(C(=O)N[C@H]2CC[C@H](O[Si](C)(C)C(C)(C)C)CC2)ccc1Br]

4-Bromo-N-(trans-4-hydroxycyclohexyl)-3-methylbenzamide (157.0 g, 502.9 mmol) was initially charged in 3.14 l of dimethylformamide, admixed with imidazole (65.0 g, 955.4 mmol), and pentafluorophenol (17.2 g. 93.3 mmol) and tert-butyl(chloro)dimethylsilane (106.1 g, 704.0 mmol) were added successively. The reaction mixture was stirred at RT for 4 h, then stirred into water and extracted with ethyl acetate. The collected organic phases were washed with saturated aqueous sodium hydrogencarbonate solution and with water, dried over sodium sulphate, filtered and concentrated. The residue was stirred in petroleum ether, and the solid was filtered off with suction and washed with petroleum ether. After air drying, this gave 180.2 g of the title compound, which was converted further without further purification.

LC-MS (Method 1): Rₜ=1.44 min; MS (ESIpos): m/z=426 [M+H]⁺.

### Example 167A

### N-(trans-4-{[tert-Butyl(dimethyl)silyl]oxy}cyclohexyl)-3-methyl-4-(4,4,5,5-tetramethyl-1,3,2-dioxaborolan-2-yl)benzamide

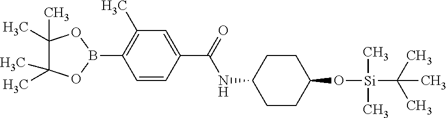 [SMILES: Cc1cc(C(=O)N[C@H]2CC[C@H](O[Si](C)(C)C(C)(C)C)CC2)ccc1B1OC(C)(C)C(C)(C)O1]

4-Bromo-N-(trans-4-{[tert-butyl(dimethyl)silyl]oxy}cyclohexyl)-3-methylbenzamide (202.0 g, 473.7 mmol) were initially charged in 2.02 l of dioxane under argon and admixed with potassium acetate (139.5 g, 1420.9 mmol) and bis(pinacolato)diboron (144.3 g, 568.4 mmol). Subsequently, 1,1′-bis(diphenylphosphino)ferrocenepalladium(II) chloride-dichloromethane complex (11.6 g, 14.2 mmol) was added and the mixture was stirred at 90° C. until conversion was complete (LC-MS reaction monitoring). Subsequently, the reaction mixture was diluted with ethyl acetate at RT and filtered through kieselguhr, and the filter residue was washed with ethyl acetate. The collected filtrate was washed with water and saturated aqueous sodium chloride solution, dried over magnesium sulphate, filtered and concentrated. The residue was dissolved in 1.8 l of dichloromethane and admixed with 300 g of silica gel (0.04-0.06 μm). The mixture was filtered with suction and the residue was washed repeatedly with dichloromethane. The collected filtrate was concentrated and the residue was recrystallized from 1.0 l of acetonitrile. This gave 162.3 g of the title compound, which was converted without further purification.

LC-MS (Method 1): Rₜ=1.57 min; MS (ESIpos): m/z=474 [M+H]⁺.

### Example 168A

### Methyl (2S)-2-{[(trans-4-{[(tert-butoxycarbonyl)amino]methyl}cyclohexyl)carbonyl]amino}-3-{4′-[(trans-4-{[tert-butyl(dimethyl)silyl]oxy}cyclohexyl)carbamoyl]-2′-methylbiphenyl-4-yl}propanoate

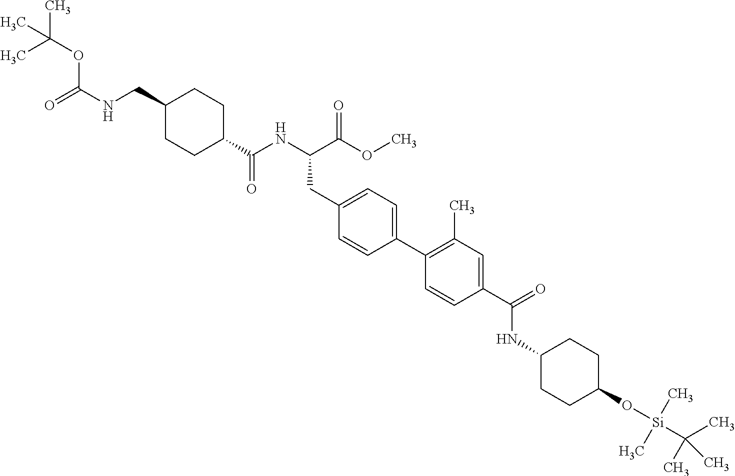 [SMILES: COC(=O)[C@H](Cc1ccc(-c2ccc(C(=O)N[C@H]3CC[C@H](O[Si](C)(C)C(C)(C)C)CC3)cc2C)cc1)NC(=O)[C@H]1CC[C@H](CNC(=O)OC(C)(C)C)CC1]

Methyl-4-bromo-N-[(trans-4-{[(tert-butoxycarbonyl)amino]methyl}cyclohexyl)carbonyl]-L-phenylalaninate (300 mg, 0.60 mmol), N-(trans-4-{[tert-butyl(dimethyl)silyl]oxy}cyclohexyl)-3-methyl-4-(4,4,5,5-tetramethyl-1,3,2-dioxaborolan-2-yl)benzamide (343 mg, 0.72 mmol) and sodium carbonate (192 mg, 1.81 mmol) were initially charged in 3.0 ml of DMF and 0.5 ml of water under argon. Subsequently, 1,1′-bis(diphenylphosphino)ferrocenepalladium(II) chloride-dichloromethane complex (49 mg, 0.06 mmol) was added and the mixture was agitated at 85° C. overnight. Subsequently, the reaction mixture was diluted with 10 ml of ethyl acetate, acidified with 1N hydrochloric acid and extracted twice with 20 ml each time of ethyl acetate. The collected organic phases were washed once each with water and saturated aqueous sodium chloride solution, dried over magnesium sulphate, filtered and concentrated. The residue was purified by means of flash silica gel chromatography (cyclohexane-ethyl acetate gradient). This gave 285 mg (61% of theory) of the title compound.

LC-MS (Method 1): Rₜ=1.47 min; MS (ESIpos): m/z=765 [M+H]⁺.

¹H NMR (400 MHz, DMSO-d₆): δ=ppm 8.21-8.09 (m, 2H), 7.73 (s, 1H), 7.70-7.64 (m, 1H), 7.32-7.20 (m, 5H), 6.81-6.72 (m, 1H), 4.57-4.48 (m, 1H), 3.79-3.68 (m, 1H), 3.62 (s, 4H), 3.14-3.05 (m, 1H), 2.98-2.88 (m, 1H), 2.78-2.70 (m, 2H), 2.24 (s, 3H), 2.10-2.00 (m, 1H), 1.88-1.79 (m, 4H), 1.71-1.58 (m, 3H), 1.57-1.48 (m, 1H), 1.47-1.04 (m, 16H), 0.90-0.70 (m, 11H), 0.08-0.03 (m, 6H).

### Example 169A

### (2S)-2-{[(trans-4-{[(tert-Butoxycarbonyl)amino]methyl}cyclohexyl)carbonyl]amino}-3-{4′-[(trans-4-{[tert-butyl(dimethyl)silyl]oxy}cyclohexyl)carbamoyl]-2′-methylbiphenyl-4-yl}propanoic acid

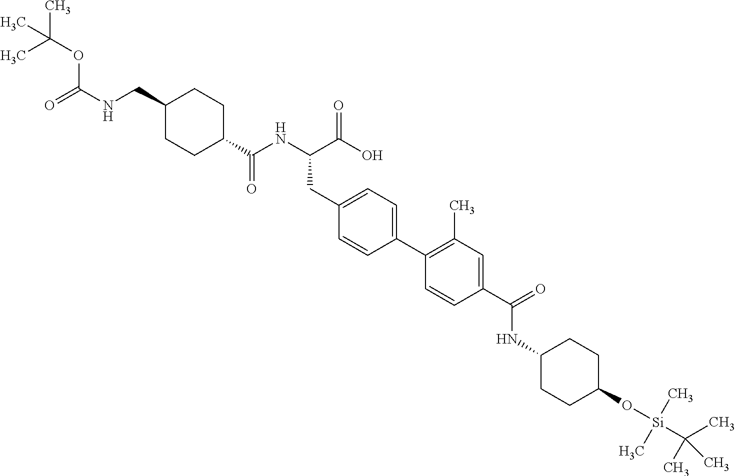 [SMILES: Cc1cc(C(=O)N[C@H]2CC[C@H](O[Si](C)(C)C(C)(C)C)CC2)ccc1-c1ccc(C[C@H](NC(=O)[C@H]2CC[C@H](CNC(=O)OC(C)(C)C)CC2)C(=O)O)cc1]

Methyl (2S)-2-{[(trans-4-{[(tert-butoxycarbonyl)amino]methyl}cyclohexyl)carbonyl]amino}-3-{4′-[(trans-4-{[tert-butyl(dimethyl)silyl]oxy}cyclohexyl)carbamoyl]-2′-methylbiphenyl-4-yl}propanoate (266 mg, 0.35 mmol) was initially charged in 2.0 ml of THF. Subsequently, a solution of lithium hydroxide (42 mg, 1.74 mmol) in 1.0 ml of water was added and the mixture was stirred at RT for 1 h. The reaction mixture was subsequently concentrated, and the residue was diluted with 20 ml of water, adjusted to pH 3-4 with 1M hydrochloric acid and stirred for 15 min. The precipitate formed was filtered off, washed with water and dried under high vacuum. This gave 236 mg (86% of theory) of the title compound.

LC-MS (Method 1): Rₜ=1.41 min; MS (ESIpos): m/z=751 [M+H]⁺.

¹H NMR (400 MHz, DMSO-d₆): δ=ppm 8.13 (d, 1H), 7.91-7.80 (m, 1H), 7.72 (s, 1H), 7.67 (d, 1H), 7.31-7.18 (m, 5H), 6.79-6.72 (m, 1H), 4.44-4.33 (m, 1H), 3.80-3.68 (m, 1H), 3.65-3.54 (m, 1H), 3.16-3.08 (m, 1H), 2.95-2.86 (m, 1H), 2.73 (t, 2H), 2.25 (s, 3H), 2.09-1.97 (m, 1H), 1.89-1.79 (m, 4H), 1.71-1.58 (m, 3H), 1.57-1.48 (m, 1H), 1.47-1.06 (m, 16H), 0.92-0.73 (m, 11H), 0.06 (s, 6H).

### Example 170A

### Methyl 3-(5-{4-[(2-{[(trans-4-{[(tert-butoxycarbonyl)amino]methyl}cyclohexyl)carbonyl]amino}-3-{4′-[(trans-4-{[tert-butyl(dimethyl)silyl]oxy}cyclohexyl)carbamoyl]-2′-methylbiphenyl-4-yl}propanoyl)amino]phenyl}-4H-1,2,4-triazol-3-yl)-2,2,3,3-tetrafluoropropanoate (enantiomer mixture)

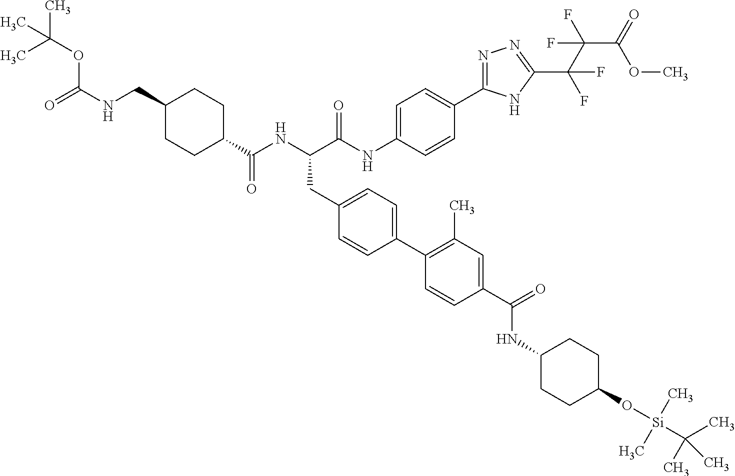 [SMILES: COC(=O)C(F)(F)C(F)(F)c1nnc(-c2ccc(NC(=O)[C@H](Cc3ccc(-c4ccc(C(=O)N[C@H]5CC[C@H](O[Si](C)(C)C(C)(C)C)CC5)cc4C)cc3)NC(=O)[C@H]3CC[C@H](CNC(=O)OC(C)(C)C)CC3)cc2)[nH]1]

(2S)-2-{[(trans-4-{[(tert-Butoxycarbonyl)amino]methyl}cyclohexyl)-carbonyl]amino}-3-{4′-[(trans-4-{[tert-butyl(dimethyl)silyl]oxy}cyclohexyl)carbamoyl]-2′-methylbiphenyl-4-yl}propanoic acid (28.00 g, 37.33 mmol) and pentafluorophenol (17.18 g. 93.32 mmol) were initially charged in 500 ml of DMF and then 4-dimethylaminopyridine (0.46 g, 3.73 mmol) was added. The mixture was cooled to −18° C. and admixed with 1-(3-dimethylaminopropyl)-3-ethylcarbodiimide hydrochloride (9.30 g, 48.53 mmol). Subsequently, the reaction mixture was warmed to RT and stirred overnight. Methyl 3-[5-(4-aminophenyl)-1H-1,2,4-triazol-3-yl]-2,2,3,3-tetrafluoropropanoate (13.07 g, 41.06 mmol) was then added to the reaction solution and the mixture was stirred at RT for 6 days. Subsequently, the reaction mixture was stirred gradually into 3 l of water, and the precipitate was filtered off and washed with water. The filter residue was taken up in 250 ml of acetonitrile and stirred at 45° C. for 15 min. The solid formed was filtered off at RT and dried under high vacuum. This gave 23.00 g (59% of theory) of the title compound as an enantiomer mixture.

LC-MS (Method 1): Rₜ=1.46 min; MS (ESIpos): m/z=1051 [M+H]⁺.

¹H NMR (400 MHz, DMSO-d₆): δ=ppm 15.16 (br. s., 1H), 10.39 (s, 1H), 8.19-8.10 (m, 2H), 7.98-7.92 (m, 2H), 7.81-7.75 (m, 2H), 7.74-7.70 (m, 1H), 7.70-7.65 (m, 1H), 7.41-7.33 (m, 2H), 7.29-7.18 (m, 3H), 6.80-6.72 (m, 1H), 4.78-4.69 (m, 1H), 3.95 (s, 3H), 3.80-3.68 (m, 1H), 3.65-3.55 (m, 1H), 3.16-3.07 (m, 1H), 2.99-2.88 (m, 1H), 2.78-2.72 (m, 2H), 2.21 (s, 3H), 2.16-2.06 (m, 1H), 1.89-1.79 (m, 4H), 1.73-1.59 (m, 3H), 1.59-1.49 (m, 1H), 1.44-1.09 (m, 18H), 0.87 (s, 11H), 0.06 (s, 6H).

### Example 171A

### Methyl 3-[5-(4-{[(2S)-2-{[(trans-4-{[(tert-butoxycarbonyl)amino]methyl}cyclohexyl)carbonyl]-amino}-3-{4′-[(trans-4-{[tert-butyl(dimethyl)silyl]oxy}cyclohexyl)carbamoyl]-2′-methylbiphenyl-4-yl}propanoyl]amino}phenyl)-4H-1,2,4-triazol-3-yl]-2,2,3,3-tetrafluoropropanoate (enantiomer 1)

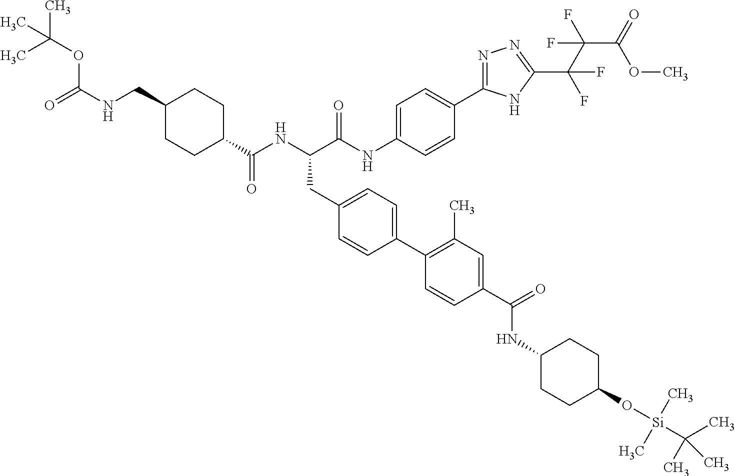 [SMILES: COC(=O)C(F)(F)C(F)(F)c1nnc(-c2ccc(NC(=O)[C@H](Cc3ccc(-c4ccc(C(=O)N[C@H]5CC[C@H](O[Si](C)(C)C(C)(C)C)CC5)cc4C)cc3)NC(=O)[C@H]3CC[C@H](CNC(=O)OC(C)(C)C)CC3)cc2)[nH]1]

Enantiomer separation of 18 g of the enantiomer mixture from Example 170A gave 6.68 g of the Example 171A title compound (enantiomer 1).

Chiral analytical HPLC: Rₜ=6.94 min; >98% ee.

Specific rotation: [α]=39.5° (c=0.447 g/100 ml, methanol, 20° C., 589 nm).

Separation method (SFC): column: Chiralpak OD-I 20 μm 400 mm×50 mm; eluent: 65% carbon dioxide, 35% methanol; temperature: 20° C.; flow rate: 400 g/min; pressure: 80 bar, UV detection: 210 nm.

Analysis (SFC): column: IC-3 5 μm 250 mm×4.6 mm; eluent: 70% carbon dioxide, 30% ethanol; temperature: 40° C.; flow rate: 3 ml/min; UV detection: 210 nm.

### Example 172A

### Methyl 3-[5-(4-{[(2S)-2-{[(trans-4-{[(tert-butoxycarbonyl)amino]methyl}cyclohexyl)carbonyl]-amino}-3-{4′-[(trans-4-{[tert-butyl(dimethyl)silyl]oxy}cyclohexyl)carbamoyl]-2′-methylbiphenyl-4-yl}propanoyl]amino}phenyl)-4H-1,2,4-triazol-3-yl]-2,2,3,3-tetrafluoropropanoate (enantiomer 2)

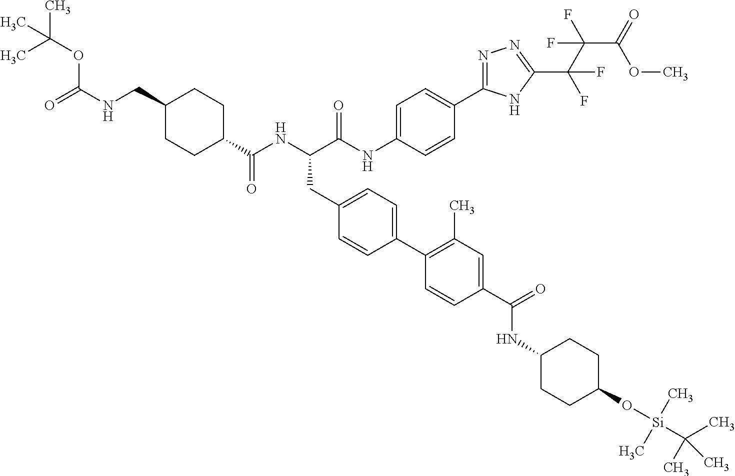 [SMILES: COC(=O)C(F)(F)C(F)(F)c1nnc(-c2ccc(NC(=O)[C@H](Cc3ccc(-c4ccc(C(=O)N[C@H]5CC[C@H](O[Si](C)(C)C(C)(C)C)CC5)cc4C)cc3)NC(=O)[C@H]3CC[C@H](CNC(=O)OC(C)(C)C)CC3)cc2)[nH]1]

Enantiomer separation of 18 g of the enantiomer mixture from Example 170A gave 6.57 g of the title compound Example 172A (enantiomer 2).

Chiral analytical HPLC: Rₜ=15.64 min; >98% ee.

Separation method (SFC): column: Chiralpak OD-I 20 μm 400 mm×50 mm; eluent: 65% carbon dioxide, 35% methanol; temperature: 20° C.; flow rate: 400 g/min; pressure: 80 bar, UV detection: 210 nm.

Analysis (SFC): column: IC-3 5 μm 250 mm×4.6 mm; eluent: 70% carbon dioxide, 30% ethanol; temperature: 40° C.; flow rate: 3 ml/min; UV detection: 210 nm.

### Example 173A

### 3-[5-(4-{[(2S)-2-{[(trans-4-{[(tert-Butoxycarbonyl)amino]methyl}cyclohexyl)carbonyl]amino}-3-{4′-[(trans-4-{[tert-butyl(dimethyl)silyl]oxy}cyclohexyl)carbamoyl]-2′-methylbiphenyl-4-yl}propanoyl]amino}phenyl)-4H-1,2,4-triazol-3-yl]-2,2,3,3-tetrafluoropropanoic acid

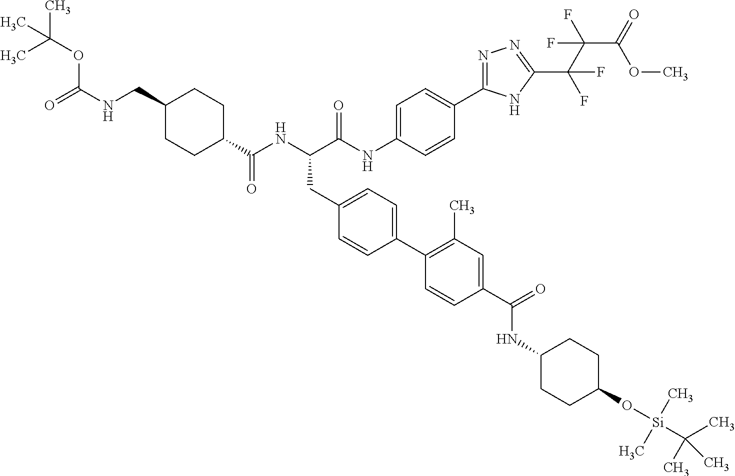 [SMILES: COC(=O)C(F)(F)C(F)(F)c1nnc(-c2ccc(NC(=O)[C@H](Cc3ccc(-c4ccc(C(=O)N[C@H]5CC[C@H](O[Si](C)(C)C(C)(C)C)CC5)cc4C)cc3)NC(=O)[C@H]3CC[C@H](CNC(=O)OC(C)(C)C)CC3)cc2)[nH]1]

Methyl 3-{5-[4-({4-bromo-N-[(trans-4-{[(tert-butoxycarbonyl)amino]methyl}cyclohexyl)carbonyl]-L-phenylalanyl}amino)phenyl]-4H-1,2,4-triazol-3-yl}-2,2,3,3-tetrafluoropropanoate (9.9 g, 12.7 mmol) and N-(trans-4-([tert-butyl(dimethyl)silyl]oxy}cyclohexyl)-3-methyl-4-(4,4,5,5-tetramethyl-1,3,2-dioxaborolan-2-yl)benzamide (9 g, 19 mmol) were dissolved in 150 ml of dimethylformamide and admixed with aqueous sodium carbonate solution (2M, 32 ml, 63 mmol) and degassed. After adding 1 g (1.27 mmol) of 1,1′-bis(diphenylphosphino)ferrocenepalladium(II) chloride, the reaction mixture was stirred at 85° C. for 4 h. Aqueous sodium carbonate solution (2M, 12.6 ml, 25 mmol) and 0.1 g (0.13 mmol) of 1,1′-bis(diphenylphosphino)ferrocenepalladium(II) chloride were added, and the reaction mixture was stirred at 85° C. for 2 h. The mixture was stirred in water and adjusted to pH 4 with dilute acetic acid, and ethyl acetate was added until the product had precipitated. The residue was filtered off, washed with ethyl acetate and water, and dried under high vacuum. This gave 5.7 g (43% of theory) of the title compound.

LC-MS (Method 19): Rₜ=6.86 min; MS (ESIpos): m/z=1036.5 [M+H]⁺.

### Example 174A

### 3-[5-(4-{[(2S)-2-{[(trans-4-{[(tert-Butoxycarbonyl)amino]methyl}cyclohexyl)carbonyl]amino}-3-(4′-{[(3R,5S)-5-(hydroxymethyl)-2-oxopyrrolidin-3-yl]carbamoyl}-2′-methylbiphenyl-4-yl)propanoyl]amino}phenyl)-1H-1,2,4-triazol-3-yl]-2,2,3,3-tetrafluoropropanoic acid

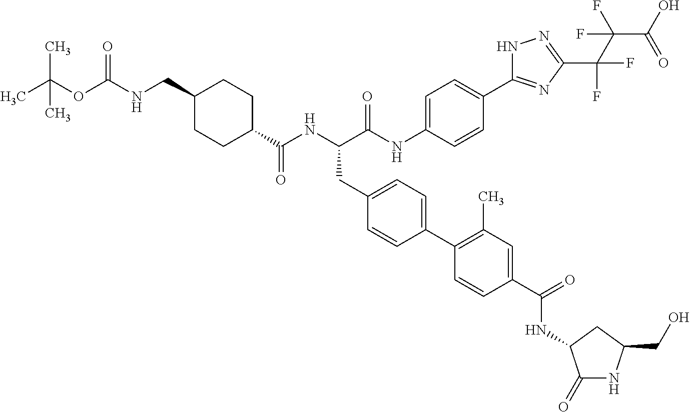 [SMILES: Cc1cc(C(=O)N[C@@H]2C[C@@H](CO)NC2=O)ccc1-c1ccc(C[C@H](NC(=O)[C@H]2CC[C@H](CNC(=O)OC(C)(C)C)CC2)C(=O)Nc2ccc(-c3nc(C(F)(F)C(F)(F)C(=O)O)n[nH]3)cc2)cc1]

4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxo-propyl]-2-methylbiphenyl-4-carboxylic acid (100 mg, 0.12 mmol) and (3R,5S)-3-amino-5-(hydroxymethyl)pyrrolidin-2-one hydrochloride (24 mg, 0.15 mmol) were dissolved in 0.74 ml of dimethylformamide and admixed with N,N-diisopropylethylamine (0.06 ml, 0.36 mmol) and N-[(dimethylamino)(3H-[1,2,3]triazolo[4,5-b]pyridin-3-yloxy)methylidene]-N-methylmethanaminium hexafluorophosphate (69 mg, 0.18 mmol) and stirred at RT for 2 h. The reaction mixture was filtered through a Millipore filter and purified via preparative HPLC (eluent: acetonitrile/water with 0.1% trifluoroacetic acid (gradient)). This gave 22 mg (5% of theory) of the title compound.

LC-MS (Method 1): Rₜ=0.83 min; MS (ESIpos): m/z=937.6 [M+H]⁺.

### Example 175A

### 3-[5-(4-{[(2S)-2-{[(trans-4-{[(tert-Butoxycarbonyl)amino]methyl}cyclohexyl)carbonyl]amino}-3-(4′-{[1-(tert-butoxycarbonyl)-3,3-dimethylpiperidin-4-yl]carbamoyl}-2′-methylbiphenyl-4-yl)propanoyl]amino}phenyl)-4H-1,2,4-triazol-3-yl]-2,2,3,3-tetrafluoropropanoic acid

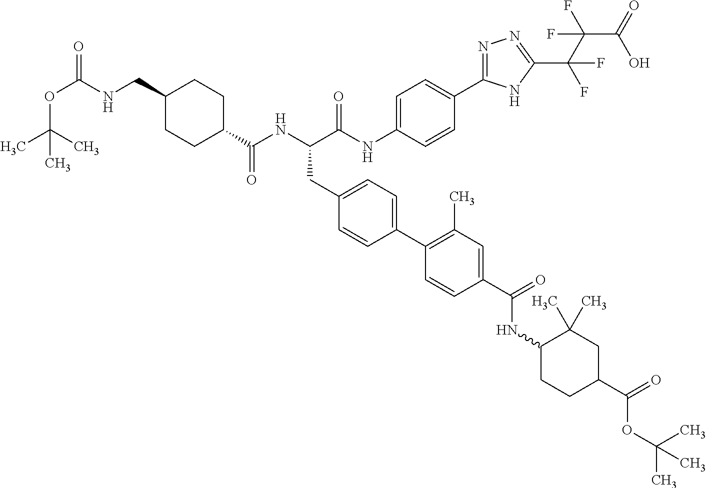 [SMILES: Cc1cc(C(=O)NC2CCC(C(=O)OC(C)(C)C)CC2(C)C)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxo-propyl]-2-methylbiphenyl-4-carboxylic acid (253 mg, 0.31 mmol) and tert-butyl 4-amino-3,3-dimethylpiperidine-1-carboxylate (140 mg, 0.61 mmol) were dissolved in 2.5 ml of dimethylformamide and admixed with N,N-diisopropylethylamine (0.21 ml, 1.2 mmol) and N-[(dimethylamino)(3H-[1,2,3]triazolo[4,5-b]pyridin-3-yloxy)methylidene]-N-methylmethanaminium hexafluorophosphate (175 mg, 0.46 mmol) and stirred at RT for 18 h. The reaction mixture was filtered through a Millipore filter and purified via preparative HPLC (eluent: acetonitrile/water with 0.1% trifluoroacetic acid (gradient)). This gave 157 mg (42% of theory) of the title compound.

LC-MS (Method 16): Rₜ=13.03 min; MS (ESIpos): m/z=1035.495 [M+H]⁺.

### Example 176A

### 3-[5-(4-{[(2S)-2-{[(trans-4-{[(tert-Butoxycarbonyl)amino]methyl}cyclohexyl)carbonyl]amino}-3-(2′-methyl-4′-{[(1-methylpiperidin-4-yl)methyl]carbamoyl}biphenyl-4-yl)propanoyl]amino}phenyl)-4H-1,2,4-triazol-3-yl]-2,2,3,3-tetrafluoropropanoic acid

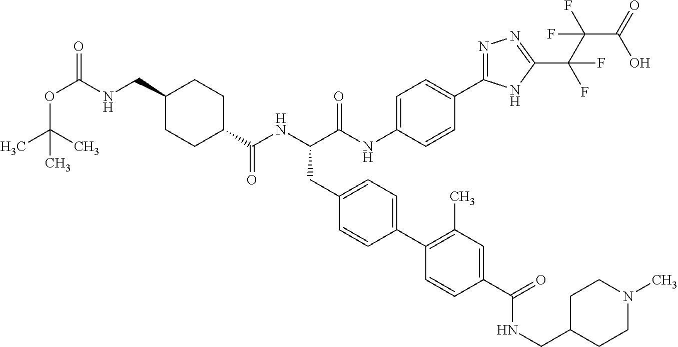 [SMILES: Cc1cc(C(=O)NCC2CCN(C)CC2)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-5-yl]phenyl}amino)-3-oxo-propyl]-2-methylbiphenyl-4-carboxylic acid (100 mg, 0.12 mmol) and 1-(1-methylpiperidin-4-yl)methanamine (19 mg, 0.15 mmol) were dissolved in 1 ml of dimethylformamide and admixed with N,N-diisopropylethylamine (0.08 ml, 0.49 mmol) and N-[(dimethylamino)(3H-[1,2,3]triazolo[4,5-b]pyridin-3-yloxy)methylidene]-N-methylmethanaminium hexafluorophosphate (69 mg, 0.18 mmol) and stirred at RT for 18 h. The reaction mixture was filtered through a Millipore filter and purified via preparative HPLC (eluent: acetonitrile/water with 0.1% trifluoroacetic acid (gradient)). This gave 65 mg (35% of theory) of the title compound.

LC-MS (Method 17): Rₜ=1.05 min; MS (ESIpos): m/z=935.445 [M+H]⁺.

### Example 177A

### tert-Butyl [(trans-4-{[(2S)-3-{2′-methyl-4′-[(1-methylpiperidin-4-yl)carbamoyl]biphenyl-4-yl}-1-oxo-1-({4-[3-(1,1,2,2-tetrafluoro-3-hydroxypropyl)-1H-1,2,4-triazol-5-yl]phenyl}amino)propan-2-yl]carbamoyl}cyclohexyl)methyl]carbamate

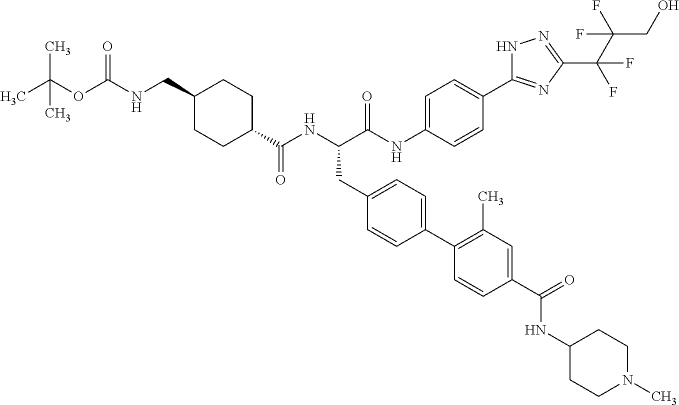 [SMILES: Cc1cc(C(=O)NC2CCN(C)CC2)ccc1-c1ccc(C[C@H](NC(=O)[C@H]2CC[C@H](CNC(=O)OC(C)(C)C)CC2)C(=O)Nc2ccc(-c3nc(C(F)(F)C(F)(F)CO)n[nH]3)cc2)cc1]

4′-[(2S)-2-{[(trans-4-{[(tert-Butoxycarbonyl)amino]methyl}cyclohexyl)carbonyl]amino}-3-oxo-3-({4-[3-(1, 1,2,2-tetrafluoro-3-hydroxypropyl)-1H-1,2,4-triazol-5-yl]phenyl}amino)propyl]-2-methylbiphenyl-4-carboxylic acid (80 mg, 0.1 mmol) and 1-methylpiperidin-4-amine (17 mg, 0.15 mmol) were dissolved in 1 ml of dimethylformamide and admixed with N,N-diisopropylethylamine (0.05 ml, 0.30 mmol) and N-[(dimethylamino)(3H-[1,2,3]triazolo[4,5-b]pyridin-3-yloxy)methylidene]-N-methylmethanaminium hexafluorophosphate (56 mg, 0.15 mmol) and stirred at RT for 18 h. The reaction mixture was filtered through a Millipore filter and purified via preparative HPLC (eluent: acetonitrile/water with 0.1% trifluoroacetic acid (gradient)). This gave 57 mg (55% of theory) of the title compound.

LC-MS (Method 1): Rₜ=0.82 min; MS (ESIpos): m/z=907.5 [M+H]⁺.

### Example 178A

### tert-Butyl [(trans-4-{[(2S)-3-{4′-[(trans-4-hydroxycyclohexyl)carbamoyl]-2′-methylbiphenyl-4-yl}-1-oxo-1-({4-[3-(1,1,2,2-tetrafluoro-3-hydroxypropyl)-1H-1,2,4-triazol-5-yl]phenyl}amino)-propan-2-yl]carbamoyl}cyclohexyl)methyl]carbamate

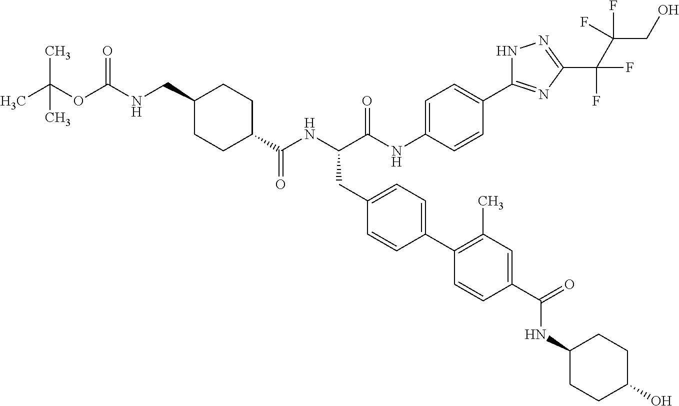 [SMILES: Cc1cc(C(=O)N[C@H]2CC[C@H](O)CC2)ccc1-c1ccc(C[C@H](NC(=O)[C@H]2CC[C@H](CNC(=O)OC(C)(C)C)CC2)C(=O)Nc2ccc(-c3nc(C(F)(F)C(F)(F)CO)n[nH]3)cc2)cc1]

4′-[(2S)-2-{[(trans-4-{[(tert-Butoxycarbonyl)amino]methyl}cyclohexyl)carbonyl]amino}-3-oxo-3-({4-[3-(1,1,2,2-tetrafluoro-3-hydroxypropyl)-1H-1,2,4-triazol-5-yl]phenyl}amino)propyl]-2-methylbiphenyl-4-carboxylic acid (80 mg, 0.1 mmol) and trans-4-aminocyclohexanol (17 mg, 0.15 mmol) were dissolved in 1 ml of dimethylformamide and admixed with N,N-diisopropylethylamine (0.05 ml, 0.3 mmol) and N-[(dimethylamino)(3H-[1,2,3]triazolo[4,5-b]pyridin-3-yloxy)methylidene]-N-methylmethanaminium hexafluorophosphate (56 mg, 0.15 mmol) and stirred at RT for 18 h. The reaction mixture was filtered through a Millipore filter and purified via preparative HPLC (eluent: acetonitrile/water with 0.1% trifluoroacetic acid (gradient)). This gave 61 mg (59% of theory) of the title compound.

LC-MS (Method 1): Rₜ=1.00 min; MS (ESIpos): m/z=808.5 [M+H]⁺.

### Example 179A

### 3-[3-(4-{[(2S)-2-{[(trans-4-{[(tert-Butoxycarbonyl)amino]methyl}cyclohexyl)carbonyl]amino}-3-(3′-fluoro-4′-{[(3R)-2-oxopiperidin-3-yl]carbamoyl}biphenyl-4-yl)propanoyl]amino}phenyl)-1H-1,2,4-triazol-5-yl]-2,2,3,3-tetrafluoropropanoic acid

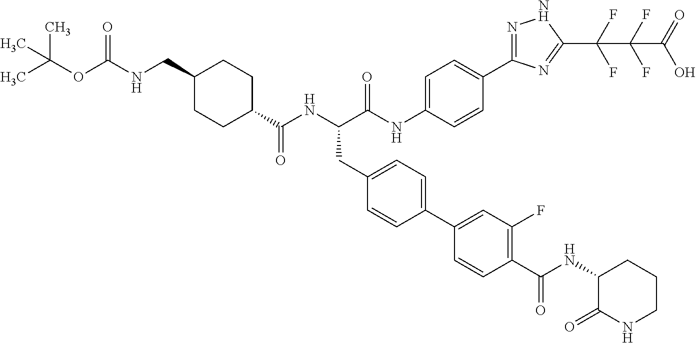 [SMILES: CC(C)(C)OC(=O)NC[C@H]1CC[C@H](C(=O)N[C@@H](Cc2ccc(-c3ccc(C(=O)N[C@@H]4CCCNC4=O)c(F)c3)cc2)C(=O)Nc2ccc(-c3n[nH]c(C(F)(F)C(F)(F)C(=O)O)n3)cc2)CC1]

4′-[(2S)-2-{[(trans-4-{[(tert-Butoxycarbonyl)amino]methyl}cyclohexyl)carbonyl]amino}-3-({4-[5-(2-carboxy-1,1,2,2-tetrafluoroethyl)-1H-1,2,4-triazol-3-yl]phenyl}amino)-3-oxopropyl-3-fluorobiphenyl-4-carboxylic acid (135 mg, 0.16 mmol) and (3R)-3-aminopiperidin-2-one (24 mg, 0.21 mmol) were dissolved in 4.6 ml of dimethylformamide and admixed with N,N-diisopropylethylamine (0.09 ml, 0.49 mmol) and N-[(dimethylamino)(3H-[1,2,3]triazolo[4,5-b]pyridin-3-yloxy)methylidene]-N-methylmethanaminium hexafluorophosphate (92 mg, 0.24 mmol) and stirred at RT for 18 h. The reaction mixture was filtered through a Millipore filter and purified via preparative HPLC (eluent: acetonitrile/water with 0.1% trifluoroacetic acid (gradient)). This gave 52 mg (26% of theory) of the title compound.

LC-MS (Method 1): Rₜ=0.91 min; MS (ESIpos): m/z=925.3 [M+H]⁺.

### Example 180A

### 3-[3-(4-{[(2S)-2-{[(trans-4-{[(tert-Butoxycarbonyl)amino]methyl}cyclohexyl)carbonyl]amino}-3-{3′-fluoro-4′-[(trans-4-hydroxycyclohexyl)carbamoyl]biphenyl-4-yl}propanoyl]amino}phenyl)-1H-1,2,4-triazol-5-yl]-2,2,3,3-tetrafluoropropanoic acid

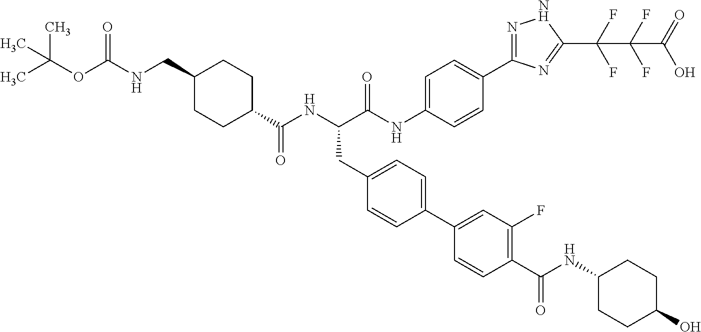 [SMILES: CC(C)(C)OC(=O)NC[C@H]1CC[C@H](C(=O)N[C@@H](Cc2ccc(-c3ccc(C(=O)N[C@H]4CC[C@H](O)CC4)c(F)c3)cc2)C(=O)Nc2ccc(-c3n[nH]c(C(F)(F)C(F)(F)C(=O)O)n3)cc2)CC1]

4′-[(2S)-2-{[(trans-4-{[(tert-Butoxycarbonyl)amino]methyl}cyclohexyl)carbonyl]amino}-3-({4-[5-(2-carboxy-1, 1,2,2-tetrafluoroethyl)-1H-1,2,4-triazol-3-yl]phenyl}amino)-3-oxopropyl]-3-fluorobiphenyl-4-carboxylic acid (100 mg, 0.12 mmol) and trans-4-aminocyclohexanol (18 mg, 0.16 mmol) were dissolved in 3.3 ml of dimethylformamide and admixed with N,N-diisopropylethylamine (0.06 ml, 0.36 mmol) and N-[(dimethylamino)(3H-[1,2,3]triazolo[4,5-b]pyridin-3-yloxy)methylidene]-N-methylmethanaminium hexafluorophosphate (69 mg, 0.18 mmol) and stirred at RT for 18 h. The reaction mixture was filtered through a Millipore filter and purified via preparative HPLC (eluent: acetonitrile/water with 0.1% trifluoroacetic acid (gradient)). This gave 17 mg of the title compound.

LC-MS (Method 1): Rₜ=0.99 min; MS (ESIpos): m/z=926.7 [M+H]⁺.

### Example 181A

### 3-[5-(4-{[(2S)-2-{[(trans-4-{[(tert-Butoxycarbonyl)amino]methyl}cyclohexyl)carbonyl]amino}-3-{2′-methyl-4′-[methyl(1-methylpiperidin-4-yl)carbamoyl]biphenyl-4-yl}propanoyl]amino}phenyl)-4H-1,2,4-triazol-3-yl]-2,2,3,3-tetrafluoropropanoic acid

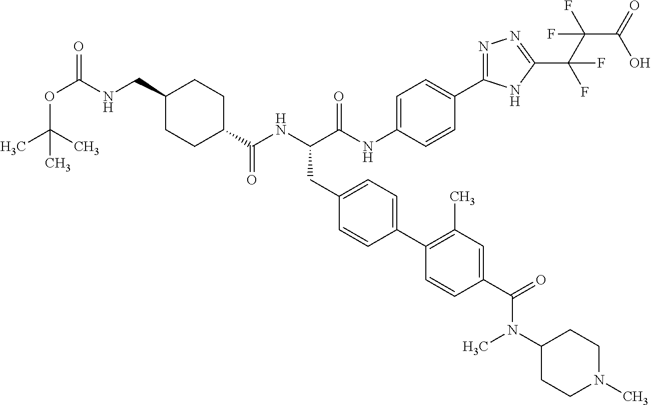 [SMILES: Cc1cc(C(=O)N(C)C2CCN(C)CC2)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

4′-[(2S)-2-{[(trans-4-{[(tert-Butoxycarbonyl)amino]methyl}cyclohexyl)carbonyl]amino}-3-({4-[5-(2-carboxy-1,1,2,2-tetrafluoroethyl)-4H-1,2,4-triazol-3-yl]phenyl}amino)-3-oxopropyl]-2-methylbiphenyl-4-carboxylic acid (100 mg, 0.12 mmol) and N,1-dimethylpiperidin-4-amine (18.6 mg, 0.15 mmol) were dissolved in 1 ml of dimethylformamide and admixed with N,N-diisopropylethylamine (0.08 ml, 0.49 mmol) and N-[(dimethylamino)(3H-[1,2,3]triazolo[4,5-b]pyridin-3-yloxy)methylidene]-N-methylmethanaminium hexafluorophosphate (69 mg, 0.18 mmol) and stirred at RT for 18 h. The reaction mixture was filtered through a Millipore filter and purified via preparative HPLC (eluent: acetonitrile/water with 0.1% trifluoroacetic acid (gradient)). This gave 26 mg (20% of theory) of the title compound.

LC-MS (Method 1): Rₜ=0.82 min; MS (ESIpos): m/z=935.5 [M+H]⁺.

### Example 182A

### 3-{5-[4-({(2S)-2-{[(trans-4-{[(tert-Butoxycarbonyl)amino]methyl}cyclohexyl)carbonyl]amino}-3-[4′-({2-[4-(dimethylamino)piperidin-1-yl]-2-oxoethyl}carbamoyl)-2′-methylbiphenyl-4-yl]propanoyl}amino)phenyl]-4H-1,2,4-triazol-3-yl}-2,2,3,3-tetrafluoropropanoic acid

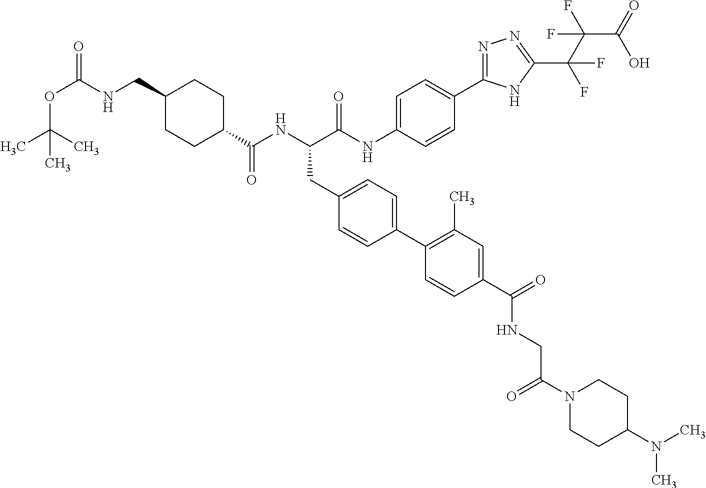 [SMILES: Cc1cc(C(=O)NCC(=O)N2CCC(N(C)C)CC2)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

4′-[(2S)-2-{[(trans-4-{[(tert-Butoxycarbonyl)amino]methyl}cyclohexyl)carbonyl]amino}-3-({4-[5-(2-carboxy-1,1,2,2-tetrafluoroethyl)-4H-1,2,4-triazol-3-yl]phenyl}amino)-3-oxopropyl]-2-methylbiphenyl-4-carboxylic acid (110 mg, 0.13 mmol) and 2-amino-1-[4-(dimethylamino)piperidin-1-yl]ethanone (29.6 mg, 0.16 mmol) were dissolved in 1 ml of dimethylformamide and admixed with N,N-diisopropylethylamine (0.09 ml, 0.53 mmol) and N-[(dimethylamino)(3H-[1,2,3]triazolo[4,5-b]pyridin-3-yloxy)methylidene]-N-methylmethanaminium hexafluorophosphate (76 mg, 0.2 mmol) and stirred at RT for 18 h. The reaction mixture was filtered through a Millipore filter and purified via preparative HPLC (eluent: acetonitrile/water with 0.1% trifluoroacetic acid (gradient)). This gave 66.8 mg (43% of theory) of the title compound.

LC-MS (Method 1): Rₜ=0.78 min; MS (ESIpos): m/z=992.6 [M+H]⁺.

### Example 183A

### 3-[5-(4-{[(2S)-2-{[(trans-4-{[(tert-Butoxycarbonyl)amino]methyl}cyclohexyl)carbonyl]amino}-3-(4′-{[1-(tert-butoxycarbonyl)octahydrocyclopenta[b]pyrrol-4-yl]carbamoyl}-2′-methylbiphenyl-4-yl)propanoyl]amino}phenyl)-4H-1,2,4-triazol-3-yl]-2,2,3,3-tetrafluoropropanoic acid

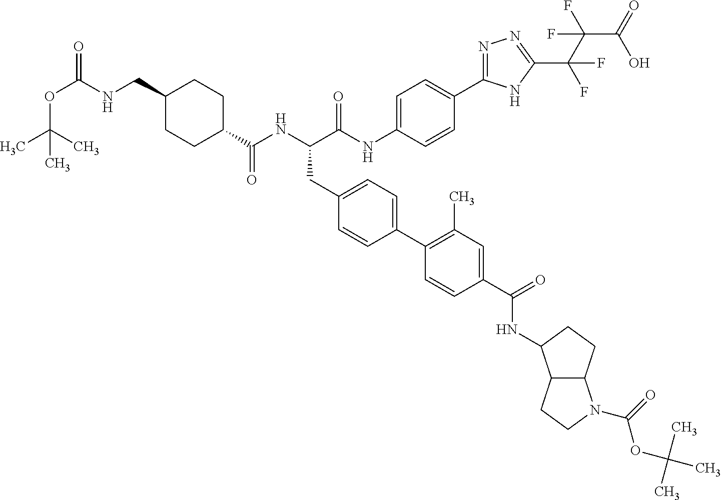 [SMILES: Cc1cc(C(=O)NC2CCC3C2CCN3C(=O)OC(C)(C)C)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

4′-[(2S)-2-{[(trans-4-{[(tert-Butoxycarbonyl)amino]methyl}cyclohexyl)carbonyl]amino}-3-({4-[3-(2-carboxy-1, 1,2,2-tetrafluoroethyl)-1H-1,2,4-triazol-3-yl]phenyl}amino)-3-oxopropyl]-2-methylbiphenyl-4-carboxylic acid (110 mg, 0.13 mmol) and tert-butyl 4-aminohexahydrocyclopenta[b]pyrrole-1(2H)-carboxylate (36.2 mg, 0.16 mmol) were dissolved in 1 ml of dimethylformamide and admixed with N,N-diisopropylethylamine (0.09 ml, 0.53 mmol) and N-[(dimethylamino)(3H-[1,2,3]triazolo[4,5-b]pyridin-3-yloxy)methylidene]-N-methylmethanaminium hexafluorophosphate (76 mg, 0.2 mmol) and stirred at RT for 18 h. The reaction mixture was filtered through a Millipore filter and purified via preparative HPLC (eluent: acetonitrile/water with 0.1% trifluoroacetic acid (gradient)). This gave 26 mg (15% of theory) of the title compound.

LC-MS (Method 1): Rₜ=1.17 min; MS (ESIpos): m/z=1033.7 [M+H]⁺.

### Example 184A

### 3-{5-[4-({(2S)-2-{[(trans-4-{[(tert-Butoxycarbonyl)amino]methyl}cyclohexyl)carbonyl]amino}-3-[4′-(isopropylcarbamoyl)-2′-methylbiphenyl-4-yl]propanoyl}amino)phenyl]-4H-1,2,4-triazol-3-yl}-2,2,3,3-tetrafluoropropanoic acid

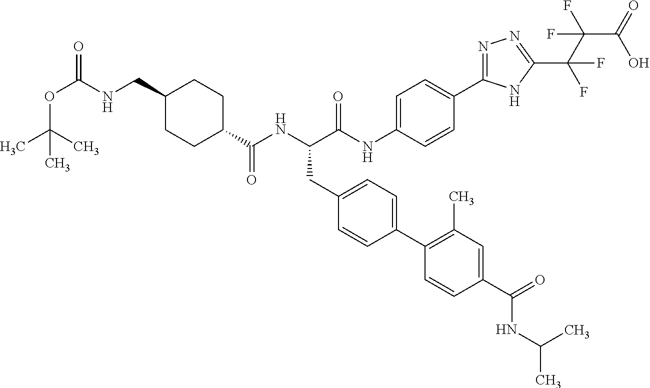 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

4′-[(2S)-2-{[(trans-4-{[(tert-Butoxycarbonyl)amino]methyl}cyclohexyl)carbonyl]amino}-3-({4-[3-(2-carboxy-1,1,2,2-tetrafluoroethyl)-1H-1,2,4-triazol-3-yl]phenyl}amino)-3-oxopropyl]-2-methylbiphenyl-4-carboxylic acid (100 mg, 0.12 mmol) and propan-2-amine (8.6 mg, 0.15 mmol) were dissolved in 1 ml of dimethylformamide and admixed with N,N-diisopropylethylamine (0.08 ml, 0.49 mmol) and N-[(dimethylamino)(3H-[1,2,3]triazolo[4,5-b]pyridin-3-yloxy)methylidene]-N-methylmethanaminium hexafluorophosphate (69 mg, 0.18 mmol) and stirred at RT for 18 h. The reaction mixture was filtered through a Millipore filter and purified via preparative HPLC (eluent: acetonitrile/water with 0.1% trifluoroacetic acid (gradient)). This gave 25 mg (17% of theory) of the title compound.

LC-MS (Method 13): Rₜ=3.02 min; MS (ESIpos): m/z=866.5 [M+H]⁺.

### Example 185A

### tert-Butyl [(trans-4-{[(2S)-3-{2′,6′-dimethoxy-4′-[(1-methylpiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]-carbamate

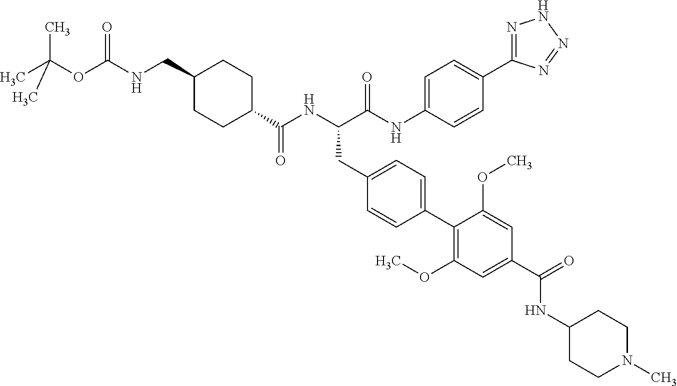 [SMILES: COc1cc(C(=O)NC2CCN(C)CC2)cc(OC)c1-c1ccc(C[C@H](NC(=O)[C@H]2CC[C@H](CNC(=O)OC(C)(C)C)CC2)C(=O)Nc2ccc(-c3nn[nH]n3)cc2)cc1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2,6-dimethoxybiphenyl-4-carboxylic acid (300 mg, 60% pure, 0.25 mmol) and 4-amino-N-methylpiperidine (56 mg, 0.50 mmol) were initially charged in 4.5 ml of dimethylformamide, admixed with N,N-diisopropylethylamine (0.13 ml, 0.74 mmol) and 1-[bis(dimethylamino)methylene]-1H-1,2,3-triazolo[4,5-b]pyridinium 3-oxide hexafluorophosphate (141 mg, 0.37 mmol) and the mixture was left to stand at RT for 3 days. The reaction mixture was diluted with 20 ml of water and acidified with 1M hydrochloric acid. The suspension thus obtained was diluted 20 ml of ethyl acetate, then filtered, and the filter residue was dried under high vacuum. The solid was taken up in acetonitrile/DMSO and filtered, and the filtrate was purified by means of preparative HPLC (eluent: acetonitrile/water with 0.1% formic acid (gradient)). This gave 68 mg (34% of theory) of the title compound.

LC-MS (Method 1): Rₜ=0.77 min; MS (ESIpos): m/z=825 [M+H]⁺.

¹H NMR (400 MHz, DMSO-d₆): δ=ppm 10.20 (s, 1H), 8.36 (d, 1H), 8.12 (d, 1H), 7.93 (d, 2H), 7.67 (d, 2H), 7.29 (d, 2H), 7.17 (s, 2H), 7.12 (d, 2H), 6.80-6.72 (m, 1H), 4.77-4.66 (m, 1H), 4.01-3.88 (m, 1H), 3.69 (s, 6H), 3.23-3.16 (m, 2H), 3.12-3.04 (m, 1H), 2.95-2.86 (m, 1H), 2.79-2.64 (m, 3H), 2.60-2.54 (m, 2H), 2.18-2.07 (m, 1H), 2.00-1.90 (m, 2H), 1.80-1.52 (m, 6H), 1.37 (s, 9H), 1.32-1.11 (m, 3H), 0.91-0.76 (m, 2H).

### Example 186A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2,6-dimethoxybiphenyl-4-carboxylic acid

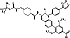 [SMILES: COc1cc(C(=O)O)cc(OC)c1-c1ccc(C[C@H](NC(=O)[C@H]2CC[C@H](CNC(=O)OC(C)(C)C)CC2)C(=O)Nc2ccc(-c3nn[nH]n3)cc2)cc1]

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (1.00 g, 1.60 mmol) from Example 4A, tetrahydroxydiborane (286 mg, 3.19 mmol) and 2′-(azanidyl-kappa-N-)biphenyl-2-yl-kappaC2][di-(3s,5s,7s)-adamantan-1-yl(butyl)phosphoranyl](methanesulphonatato-kappa-O-)palladium (116 mg, 0.16 mmol) were initially charged in 12.0 ml of methanol under argon. Subsequently, N,N-diisopropylethylamine (0.83 ml, 4.79 mmol) was added and the mixture was agitated at 50° C. for 2 h. Subsequently, 6.38 ml of 1M aqueous potassium phosphate solution and 4-bromo-3,5-dimethoxybenzoic acid (417 mg, 1.60 mmol) were added at RT and the mixture was agitated at 50° C. for 3 days. The reaction mixture was filtered through Celite and the filter residue was washed with methanol. The filtrate was concentrated to half the volume and acidified with 1M hydrochloric acid, and the solid formed was filtered off and dried under high vacuum. This gave 1.07 g of crude product (80% purity), which was converted further without further purification.

LC-MS (Method 1): Rₜ=0.92 min; MS (ESIpos): m/z=727 [M+H]⁺.

### Example 187A

### tert-Butyl 4-[({4′-[(2S)-2-{[(trans-4-{[(tert-butoxycarbonyl)amino]methyl}cyclohexyl)carbonyl]-amino}-3-oxo-3-({[4-(2H-tetrazol-5-yl)phenyl]amino}propyl]-3,5-difluorobiphenyl-4-yl}carbonyl)-amino]piperidine-1-carboxylate

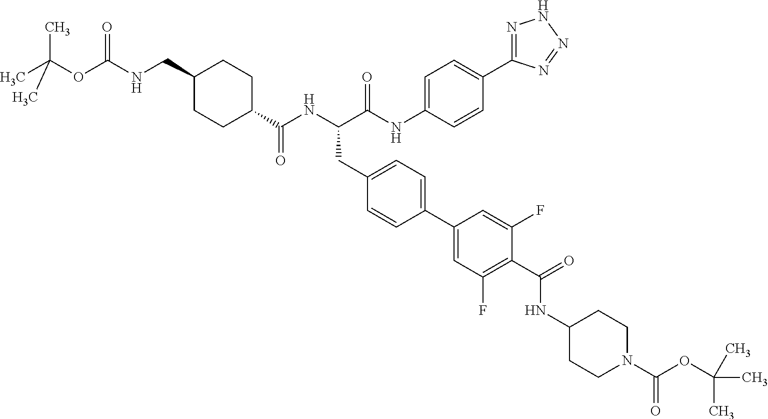 [SMILES: CC(C)(C)OC(=O)NC[C@H]1CC[C@H](C(=O)N[C@@H](Cc2ccc(-c3cc(F)c(C(=O)NC4CCN(C(=O)OC(C)(C)C)CC4)c(F)c3)cc2)C(=O)Nc2ccc(-c3nn[nH]n3)cc2)CC1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-3,5-difluorobiphenyl-4-carboxylic acid (70 mg, 0.10 mmol) was initially charged in 1.0 ml of DMF, and ter-butyl 4-aminopiperidine-1-carboxylate (40 mg, 0.20 mmol) and N,N-diisopropylethylamine (0.05 ml, 0.30 mmol) were added. Subsequently, 1-[bis(dimethylamino)methylene]-1H-1,2,3-triazolo[4,5-b]pyridinium 3-oxide hexafluorophosphate (57 mg, 0.15 mmol) was added and the mixture was agitated at RT overnight. The reaction mixture was diluted with water, acidified with 1M hydrochloric acid and extracted twice with 20 ml each time of ethyl acetate. The combined organic phases were dried over magnesium sulphate, filtered and concentrated. The residue was stirred with acetonitrile and filtered. The crude product thus obtained was separated by means of preparative HPLC (eluent: acetonitrile/water with 0.1% formic acid (gradient)). This gave 43 mg (88% of theory) of the title compound.

LC-MS (Method 1): Rₜ=1.11 min; MS (ESIpos): m/z=886 [M+H]⁺.

¹H NMR (400 MHz, DMSO-d₆): δ=ppm 16.71 (br. s., 1H), 10.45 (s, 1H), 8.70 (d, 1H), 8.16 (d, 1H), 7.99 (d, 2H), 7.82 (d, 2H), 7.72 (d, 2H), 7.51 (d, 2H), 7.42 (d, 2H), 6.80-6.71 (m, 1H), 4.76-4.66 (m, 1H), 4.01-3.89 (m, 1H), 3.88-3.80 (m, 2H), 3.15-3.06 (m, 1H), 3.01-2.85 (m, 3H), 2.79-2.71 (m, 2H), 2.16-2.05 (m, 1H), 1.87-1.75 (m, 2H), 1.75-1.50 (m, 5H), 1.32-1.08 (m, 5H), 0.92-0.73 (m, 2H).

### Example 188A

### 4′-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]-3,5-difluorobiphenyl-4-carboxylic acid

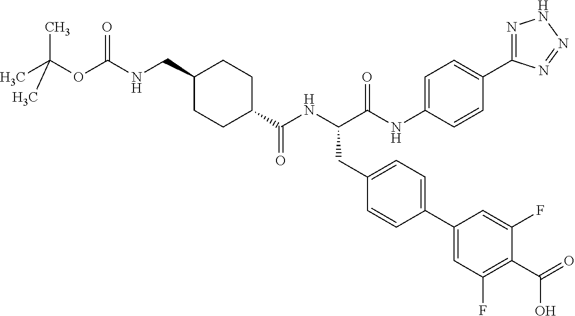 [SMILES: CC(C)(C)OC(=O)NC[C@H]1CC[C@H](C(=O)N[C@@H](Cc2ccc(-c3cc(F)c(C(=O)O)c(F)c3)cc2)C(=O)Nc2ccc(-c3nn[nH]n3)cc2)CC1]

Methyl 4′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-3,5-difluorobiphenyl-4-carboxylate (43 mg, 0.06 mmol) was initially charged in 1.0 ml of THF, then a solution of lithium hydroxide (29 mg, 1.20 mmol) in 0.5 ml of water was added and the mixture was stirred at RT overnight. The reaction mixture was then diluted with 10 ml of water and acidified with 1M hydrochloric acid. The precipitate formed was filtered off, washed with water and dried under high vacuum. This gave 36 mg of the title compound as a crude product (90% purity), which was converted further without further purification.

LC-MS (Method 1): Rₜ=0.99 min; MS (ESIpos): m/z=704 [M+H]⁺.

### Example 189A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-3,5-difluorobiphenyl-4-carboxylate

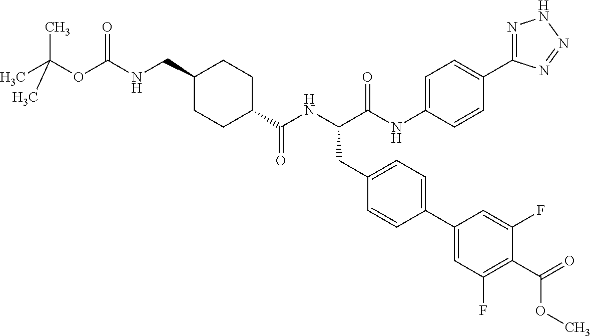 [SMILES: COC(=O)c1c(F)cc(-c2ccc(C[C@H](NC(=O)[C@H]3CC[C@H](CNC(=O)OC(C)(C)C)CC3)C(=O)Nc3ccc(-c4nn[nH]n4)cc3)cc2)cc1F]

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (250 mg, 0.40 mmol) from Example 4A, tetrahydroxydiborane (107 mg, 1.20 mmol) and 2′-(azanidyl-kappa-N-)biphenyl-2-yl-kappaC2][di-(3s,5s,7s)-adamantan-1-yl(butyl)phosphoranyl](methanesulphonatato-kappa-O-)palladium (29 mg, 0.04 mmol) were initially charged in 5.0 ml of methanol under argon. Subsequently, N,N-diisopropylethylamine (0.21 ml, 1.20 mmol) was added and the mixture was stirred at 50° C. for 3 h. Subsequently, 1.20 ml of 1M aqueous potassium phosphate solution and methyl 4-bromo-2,6-difluorobenzoate (100 mg, 0.40 mmol) were added at RT and the mixture was stirred at 50° C. overnight. The reaction mixture was then diluted with 10 ml of water and acidified with 1M hydrochloric acid, and the solid formed was filtered off. The filter residue was separated by means of preparative HPLC (eluent: acetonitrile/water with 0.1% formic acid (gradient)). This gave 46 mg of crude product (90% purity), which was converted further without further purification.

LC-MS (Method 1): Rₜ=1.09 min; MS (ESIpos): m/z=718 [M+H]⁺.

¹H NMR (400 MHz, DMSO-d₆): δ=ppm 16.65 (br. s., 1H), 10.46 (s, 1H), 8.17 (d, 1H), 7.99 (d, 2H), 7.87-7.75 (m, 3H), 7.63 (d, 2H), 7.44 (d, 2H), 6.80-6.70 (m, 1H), 4.77-4.66 (m, 1H), 3.92-3.88 (m, 1H), 3.15-3.08 (m, 1H), 2.99-2.89 (m, 1H), 2.78-2.70 (m, 2H), 2.17-2.04 (m, 1H), 1.75-1.49 (m, 4H), 1.36 (s, 9H), 1.31-1.09 (m, 3H), 0.91-0.74 (m, 2H).

### Example 190A

### tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2-ethoxybiphenyl-4-yl}carbonyl)-amino]piperidine-1-carboxylate

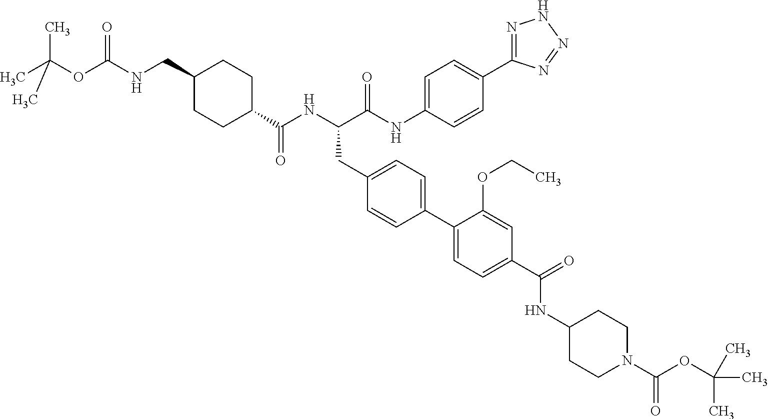 [SMILES: CCOc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-ethoxybiphenyl-4-carboxylic acid (70 mg, 0.10 mmol) was initially charged in 2.7 ml of DMF, and tert-butyl 4-aminopiperidine-1-carboxylate (107 mg, 0.53 mmol) and N,N-diisopropylethylamine (0.14 ml, 0.80 mmol) were added. Subsequently, 1-[bis(dimethylamino)methylene]-1H-1,2,3-triazolo[4,5-b]pyridinium 3-oxide hexafluorophosphate (152 mg, 0.40 mmol) was added and the mixture was agitated at RT for 2 h. The reaction mixture was diluted with water, acidified with 1M hydrochloric acid and extracted twice with 20 ml each time of ethyl acetate. The combined organic phases were dried over magnesium sulphate, filtered and concentrated. The residue was stirred with acetonitrile and filtered. The crude product thus obtained was separated by means of preparative HPLC (eluent: acetonitrile/water with 0.1% formic acid (gradient)). This gave 70 mg (28% of theory) of the title compound.

LC-MS (Method 1): Rₜ=1.11 min; MS (ESIpos): m/z=894 [M+H]⁺.

¹H NMR (400 MHz, DMSO-d₆): δ=ppm 16.71 (br. s., 1H), 10.44 (s, 1H), 8.26 (d, 1H), 8.16 (d, 1H), 7.99 (d, 2H), 7.82 (d, 2H), 7.55-7.43 (m, 4H), 7.35 (d, 3H), 6.83-6.70 (m, 1H), 4.77-4.67 (m, 1H), 4.14-3.86 (m, 5H), 3.15-3.03 (m, 1H), 2.99-2.69 (m, 6H), 2.19-2.07 (m, 1H), 1.85-1.75 (m, 2H), 1.74-1.54 (m, 4H), 1.41 (s, 9H), 1.37 (s, 9H), 1.31-1.10 (m, 6H), 0.91-0.74 (m, 2H).

### Example 191A

### 3-Ethoxy-4-(4,4,5,5-tetramethyl-1,3,2-dioxaborolan-2-yl)benzoic acid

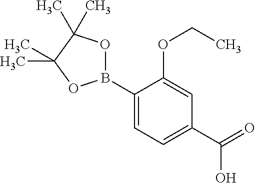 [SMILES: CCOc1cc(C(=O)O)ccc1B1OC(C)(C)C(C)(C)O1]

4-Bromo-3-ethoxybenzoic acid (1.00 g, 4.80 mmol), bis(pinacolato)diboron (1.55 g, 6.12 mmol) and 1.20 g (12.24 mmol) of potassium acetate were initially charged in 14.0 ml of dioxane under argon. Subsequently, 1,1-bis(diphenylphosphino)ferrocenedichloropalladium(II) (333 mg, 0.41 mmol) were added and the mixture was stirred at 100° C. for 10 h. Then a further 0.05 eq. of catalyst was added and the mixture was stirred at 100° C. for 4 h. Thereafter, the reaction mixture was diluted with 50 ml of ethyl acetate and filtered through Celite, and the residue was washed with ethyl acetate. The collected filtrate was concentrated and the residue was purified by means of silica gel chromatography (dichloromethane/methanol 20:1). This gave 1.74 g of the title compound as a crude product, which was converted further without further purification.

LC-MS (Method 2): R=2.19 min; MS (ESIpos): m/z=293 [M+H]⁺.

### Example 192A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-ethoxybiphenyl-4-carboxylic acid

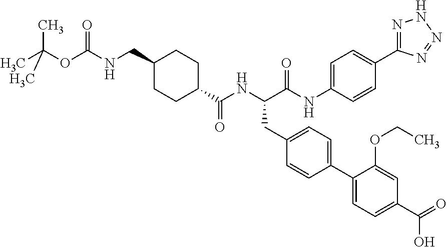 [SMILES: CCOc1cc(C(=O)O)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

3-Ethoxy-4-(4,4,5,5-tetramethyl-1,3,2-dioxaborolan-2-yl)benzoic acid (80% purity, 204 mg, 0.56 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (250 mg, 0.40 mmol) from Example 4A and 169 mg (1.60 mmol) of sodium carbonate were initially charged in a mixture of 3.0 ml of DMF and 0.3 ml of water under argon. Subsequently, 1,1-bis(diphenylphosphino)ferrocenedichloropalladium(II) (25 mg, 0.04 mmol) was added and the mixture was stirred at 150° C. in a microwave for 1 h. Thereafter, the reaction mixture was diluted with 10 ml of water, acidified with 1M hydrochloric acid and extracted twice with 20 ml each time of ethyl acetate. The combined organic phases were washed once each with water and saturated aqueous sodium chloride solution, dried over magnesium sulphate, filtered and concentrated. The residue was purified by means of preparative HPLC (eluent: acetonitrile/water with 0.1% formic acid (gradient)). This gave 43 mg of the title compound as a crude product, which was converted further without further purification.

LC-MS (Method 1): Rₜ=0.98 min; MS (ESIpos): m/z=712 [M+H]⁺.

### Example 193A

### tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-3-(trifluoromethyl)biphenyl-4-yl}carbonyl)amino]piperidine-1-carboxylate

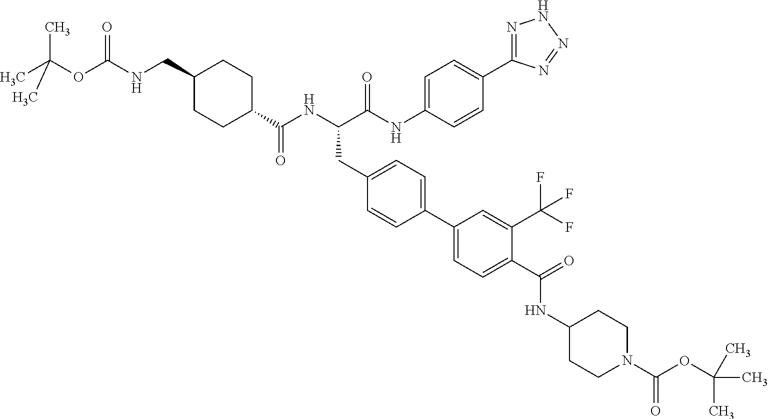 [SMILES: CC(C)(C)OC(=O)NC[C@H]1CC[C@H](C(=O)N[C@@H](Cc2ccc(-c3ccc(C(=O)NC4CCN(C(=O)OC(C)(C)C)CC4)c(C(F)(F)F)c3)cc2)C(=O)Nc2ccc(-c3nn[nH]n3)cc2)CC1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-3-(trifluoromethyl)biphenyl-4-carboxylic acid (80% purity, 190 mg, 0.21 mmol) was initially charged in 2.7 ml of DMF, and tert-butyl 4-aminopiperidine-1-carboxylate (83 mg, 0.41 mmol) and N,N-diisopropylethylamine (0.11 ml, 0.62 mmol) were added. Subsequently, 1-[bis(dimethylamino)methylene]-1H-1,2,3-triazolo[4,5-b]pyridinium 3-oxide hexafluorophosphate (118 mg, 0.31 mmol) was added and the mixture was stirred at RT for 1 h and left to stand for 2 days. The reaction mixture was diluted with acetonitrile/DMSO and separated by means of preparative HPLC (eluent: acetonitrile/water with 0.1% formic acid (gradient)). The product-containing fractions were concentrated, and the solid which precipitated was filtered off and dried under high vacuum. This gave 138 mg (73% of theory) of the title compound.

LC-MS (Method 1): Rₜ=1.16 min; MS (ESIpos): m/z=919 [M+H]⁺.

¹H NMR (400 MHz, DMSO-d₆): δ=ppm 10.46 (s, 1H), 8.47 (d, 1H), 8.17 (d, 1H), 8.04-7.91 (m, 4H), 7.82 (d, 2H), 7.70 (d, 2H), 7.56 (d, 1H), 7.45 (d, 2H), 6.80-6.70 (m, 1H), 4.77-4.68 (m, 1H), 4.00-3.81 (m, 3H), 3.17-3.07 (m, 1H), 3.01-2.83 (m, 3H), 2.78-2.71 (m, 2H), 2.16-2.06 (m, 1H), 1.85-1.76 (m, 2H), 1.75-1.50 (m, 4H), 1.40 (s, 9H), 1.36 (s, 9H), 1.34-1.08 (m, 5H), 0.90-0.74 (m, 2H).

### Example 194A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-3-(trifluoromethyl)biphenyl-4-carboxylic acid

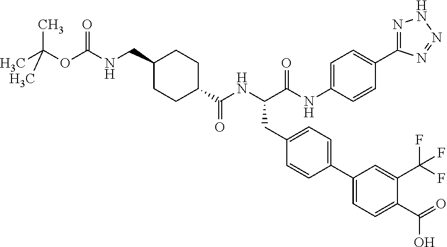 [SMILES: CC(C)(C)OC(=O)NC[C@H]1CC[C@H](C(=O)N[C@@H](Cc2ccc(-c3ccc(C(=O)O)c(C(F)(F)F)c3)cc2)C(=O)Nc2ccc(-c3nn[nH]n3)cc2)CC1]

4-(Dihydroxyboryl)-2-(trifluoromethyl)benzoic acid (523 mg, 2.23 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (1.00 g, 1.60 mmol) from Example 4A and 507 mg (4.79 mmol) of sodium carbonate were initially charged in a mixture of 1.5 ml of DMF and 0.2 ml of water under argon. Subsequently, 1,1-bis(diphenylphosphino)ferrocenedichloropalladium(II) (98 mg, 0.16 mmol) was added and the mixture was stirred at 130° C. in a microwave for 5 h. Thereafter, the reaction mixture was diluted with 20 ml of water and acidified with 1M hydrochloric acid, and the precipitated solid was filtered off and washed with water. The solid was then taken up in ethyl acetate, washed with 20 ml each of water and saturated aqueous sodium chloride solution, dried over magnesium sulphate and filtered. The filtrate was partly concentrated and the precipitate formed was filtered off. The filter residue was washed with 10 ml of ethyl acetate and dried under high vacuum. This gave 387 mg of the title compound as a crude product, which was converted further without further purification.

LC-MS (Method 1): Rₜ=1.00 min; MS (ESIpos): m/z=736 [M+H]⁺.

### Example 195A

### tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-3-fluoro-2-methoxybiphenyl-4-yl}carbonyl)amino]piperidine-1-carboxylate

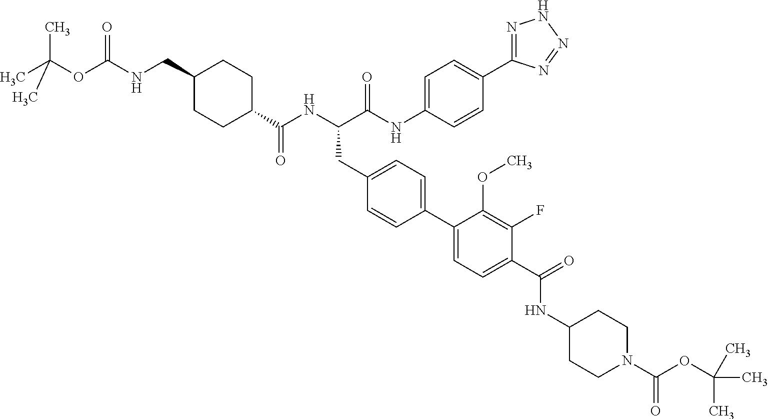 [SMILES: COc1c(-c2ccc(C[C@H](NC(=O)[C@H]3CC[C@H](CNC(=O)OC(C)(C)C)CC3)C(=O)Nc3ccc(-c4nn[nH]n4)cc3)cc2)ccc(C(=O)NC2CCN(C(=O)OC(C)(C)C)CC2)c1F]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-3-fluoro-2-methoxybiphenyl-4-carboxylic acid (130 mg, 0.18 mmol) was initially charged in 3.9 ml of DMF, and tert-butyl 4-aminopiperidine-1-carboxylate (73 mg, 0.36 mmol) and N,N-diisopropylethylamine (0.10 ml, 0.55 mmol) were added. Subsequently, 1-[bis(dimethylamino)methylene]-1H-1,2,3-triazolo[4,5-b]pyridinium 3-oxide hexafluorophosphate (104 mg, 0.27 mmol) were added and the mixture was stirred at RT overnight. The reaction mixture was diluted with acetonitrile/DMSO and filtered, and the filtrate was separated by means of preparative HPLC (eluent: acetonitrile/water with 0.1% formic acid (gradient)). This gave 34 mg (18% of theory) of the title compound.

LC-MS (Method 1): Rₜ=1.14 min; MS (ESIpos): m/z=899 [M+H]⁺.

¹H NMR (400 MHz, DMSO-d₆): δ=ppm 10.42 (s, 1H), 8.31 (d, 1H), 8.17 (d, 1H), 7.98 (d, 2H), 7.81 (d, 2H), 7.47-7.36 (m, 4H), 7.33-7.26 (m, 1H), 7.21-7.16 (m, 1H), 6.80-6.72 (m, 1H), 4.79-4.69 (m, 1H), 4.01-3.82 (m, 3H), 3.16-3.07 (m, 1H), 3.00-2.81 (m, 3H), 2.78-2.70 (m, 2H), 2.17-2.07 (m, 1H), 1.85-1.74 (m, 2H), 1.74-1.49 (m, 5H), 1.41 (s, 9H), 1.36 (s, 9H), 1.31-1.06 (m, 4H), 0.90-0.73 (m, 2H).

### Example 196A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-3-fluoro-2-methoxybiphenyl-4-carboxylic acid

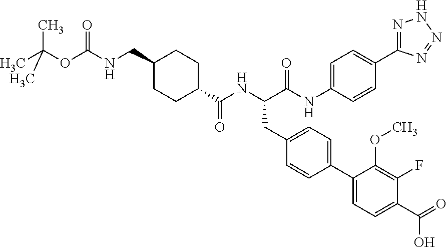 [SMILES: COc1c(-c2ccc(C[C@H](NC(=O)[C@H]3CC[C@H](CNC(=O)OC(C)(C)C)CC3)C(=O)Nc3ccc(-c4nn[nH]n4)cc3)cc2)ccc(C(=O)O)c1F]

4-(Dihydroxyboryl)-2-fluoro-3-methoxybenzoic acid (24 mg, 0.11 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (50 mg, 0.08 mmol) from Example 4A and 25 mg (0.24 mmol) of sodium carbonate were initially charged in a mixture of 1.0 ml of DMF and 0.3 ml of water under argon. Subsequently, 1,1-bis(diphenylphosphino)ferrocenedichloropalladium(II) (6 mg, 0.01 mmol) was added and the mixture was stirred at 140° C. in a microwave for 1 h. Thereafter, the reaction mixture was diluted with 20 ml of water and acidified with 1M hydrochloric acid, and the precipitated solid was filtered off and washed with water and dried under high vacuum. This gave 46 mg of the title compound as a crude product, which was converted further without further purification.

LC-MS (Method 1): Rₜ=1.95 min; MS (ESIpos): m/z=716 [M+H]⁺.

### Example 197A

### 4-(Dihydroxyboryl)-2-fluoro-3-methoxybenzoic acid

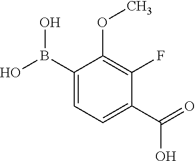

4-Bromo-2-fluoro-3-methoxybenzoic acid (100 mg, 0.42 mmol), bis(pinacolato)diboron (153 mg, 0.60 mmol) and 118 mg (1.21 mmol) of potassium acetate were initially charged in 2.5 ml of dioxane under argon. Subsequently, 1,1-bis(diphenylphosphino)ferrocenedichloropalladium(II) (16 mg, 0.02 mmol) was added and the mixture was agitated at 90° C. overnight. The reaction mixture was taken up in acetonitrile/water (1:1) and separated by means of preparative HPLC (eluent: acetonitrile/water with 0.1% formic acid (gradient)). This gave 21 mg of the title compound as a crude product, which was converted further without further purification.

LC-MS (Method 2): Rₜ=0.77 min; MS (ESIpos): m/z=215 [M+H]⁺.

### Example 198A

### tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-3-chlorobiphenyl-4-yl}carbonyl)-amino]piperidine-1-carboxylate

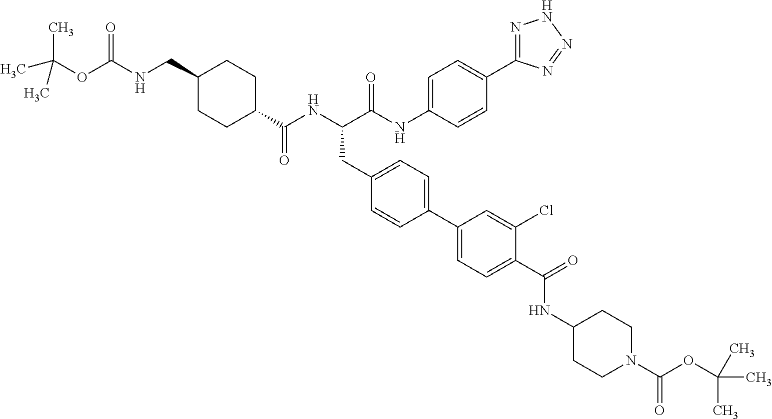 [SMILES: CC(C)(C)OC(=O)NC[C@H]1CC[C@H](C(=O)N[C@@H](Cc2ccc(-c3ccc(C(=O)NC4CCN(C(=O)OC(C)(C)C)CC4)c(Cl)c3)cc2)C(=O)Nc2ccc(-c3nn[nH]n3)cc2)CC1]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-3-chlorobiphenyl-4-carboxylic acid (90% pure, 200 mg, 0.26 mmol) was initially charged in 4.1 ml of DMF, and tert-butyl 4-aminopiperidine-1-carboxylate (103 mg, 0.51 mmol) and N,N-diisopropylethylamine (0.18 ml, 1.03 mmol) were added. Subsequently, 1-[bis(dimethylamino)methylene]-1H-1,2,3-triazolo[4,5-b]pyridinium 3-oxide hexafluorophosphate (146 mg, 0.38 mmol) was added and the mixture was stirred at RT for 1 h and left to stand overnight. The reaction mixture was diluted with water and acidified with 1M hydrochloric acid, and the precipitate formed was filtered off. The filter residue was washed with 20 ml of water and 10 ml of methyl tert-butyl ether and dried under high vacuum. The crude product thus obtained was separated by means of preparative HPLC (eluent: acetonitrile/water with 0.1% formic acid (gradient)). This gave 120 mg (53% of theory) of the title compound.

LC-MS (Method 1): Rₜ=1.13 min; MS (ESIpos): m/z=885 [M+H]⁺.

¹H NMR (400 MHz, DMSO-d₆): δ=ppm 16.72 (br. s., 1H), 10.46 (s, 1H), 8.42 (d, 1H), 8.17 (d, 1H), 7.99 (d, 2H), 7.81 (s, 1H), 7.75 (s, 1H), 7.66 (d, 3H), 7.50-7.37 (m, 3H), 6.82-6.72 (m, 1H), 4.76-4.66 (m, 1H), 4.00-3.79 (m, 3H), 3.16-3.05 (m, 1H), 3.00-2.82 (m, 3H), 2.78-2.70 (m, 2H), 2.17-2.05 (m, 1H), 1.87-1.76 (m, 2H), 1.76-1.49 (m, 4H), 1.40 (s, 9H), 1.36 (s, 9H), 1.31-1.09 (m, 4H), 0.91-0.74 (m, 2H).

### Example 199A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-3-chlorobiphenyl-4-carboxylic acid

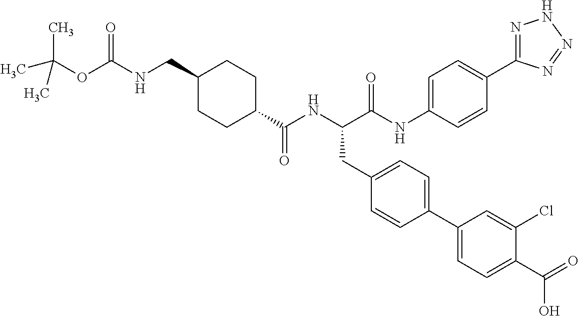 [SMILES: CC(C)(C)OC(=O)NC[C@H]1CC[C@H](C(=O)N[C@@H](Cc2ccc(-c3ccc(C(=O)O)c(Cl)c3)cc2)C(=O)Nc2ccc(-c3nn[nH]n3)cc2)CC1]

2-Chloro-4-(dihydroxyboryl)benzoic acid (448 mg, 2.23 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (1.00 g, 1.60 mmol) from Example 4A and 507 mg (4.79 mmol) of sodium carbonate were initially charged in a mixture of 15.0 ml of DMF and 2.0 ml of water under argon. Subsequently, 1,1-bis(diphenylphosphino)ferrocenedichloropalladium(II) (98 mg, 0.16 mmol) was added and the mixture was stirred at 130° C. in a microwave for 5.5 h. Thereafter, the reaction mixture was diluted with 20 ml of water and acidified with 1M hydrochloric acid, and the precipitated solid was filtered off, washed with water and dried under high vacuum. The solid thus obtained was separated by means of preparative HPLC (eluent: acetonitrile/water with 0.1% formic acid (gradient)). This gave 365 mg of the title compound as a crude product, which was converted further without further purification.

LC-MS (Method 1): Rₜ=0.97 min; MS (ESIpos): m/z=703 [M+H]⁺.

### Example 200A

### tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2,3-dimethylbiphenyl-4-yl}carbonyl)amino]piperidine-1-carboxylate

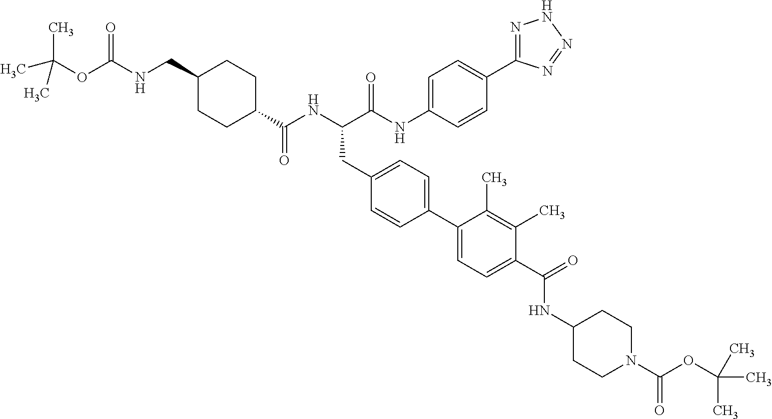 [SMILES: Cc1c(C(=O)NC2CCN(C(=O)OC(C)(C)C)CC2)ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1C]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2,3-dimethylbiphenyl-4-carboxylic acid (160 mg, 0.23 mmol) was initially charged in 3.2 ml of DMF, and tert-butyl 4-aminopiperidine-1-carboxylate (92 mg, 0.46 mmol) and N,N-diisopropylethylamine (0.16 ml, 0.92 mmol) were added. Subsequently, 1-[bis(dimethylamino)methylene]-1H-1,2,3-triazolo[4,5-b]pyridinium 3-oxide hexafluorophosphate (131 mg, 0.35 mmol) was added and the mixture was stirred at RT for 1 h and left to stand overnight. The reaction mixture was diluted with 2.0 ml of acetonitrile and separated by means of preparative HPLC (eluent: acetonitrile/water with 0.1% formic acid (gradient)). This gave 115 mg (51% of theory) of the title compound.

LC-MS (Method 1): Rₜ=1.16 min; MS (ESIpos): m/z=879 [M+H]⁺.

¹H NMR (400 MHz, DMSO-d₆): δ=ppm 16.72 (br. s., 1H), 10.40 (s, 1H), 8.21 (d, 1H), 8.15 (d, 1H), 7.99 (d, 2H), 7.81 (d, 2H), 7.36 (d, 2H), 7.16 (d, 2H), 7.10 (d, 1H), 6.99 (d, 1H), 6.80-6.73 (m, 1H), 4.80-4.71 (m, 1H), 4.00-3.83 (m, 3H), 3.16-3.08 (m, 1H), 2.98-2.82 (m, 3H), 2.78-2.72 (m, 2H), 2.24 (s, 3H), 2.16-2.09 (m, 1H), 2.07 (s, 3H), 1.85-1.75 (m, 2H), 1.73-1.58 (m, 3H), 1.57-1.48 (m, 1H), 1.40 (s, 9H), 1.37 (s, 9H), 1.36-1.05 (m, 5H), 0.90-0.75 (m, 2H).

### Example 201A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2,3-dimethylbiphenyl-4-carboxylic acid

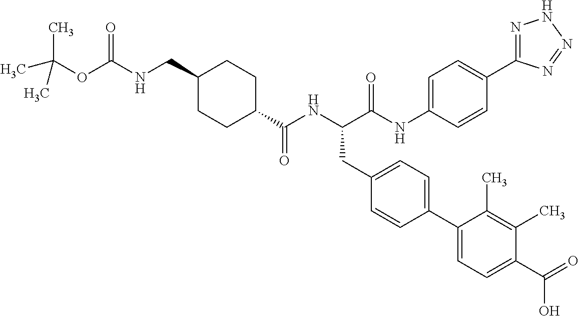 [SMILES: Cc1c(C(=O)O)ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1C]

2,3-Dimethyl-4-(4,4,5,5-tetramethyl-1,3,2-dioxaborolan-2-yl)benzoic acid (617 mg, 2.23 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (1.00 g, 1.60 mmol) from Example 4A and 507 mg (4.79 mmol) of sodium carbonate were initially charged in a mixture of 20.0 ml of DMF and 2.0 ml of water under argon. Subsequently, 1,1-bis(diphenylphosphino)ferrocenedichloropalladium(II) (98 mg, 0.16 mmol) was added and the mixture was stirred at 140° C. in a microwave for 1 h. Thereafter, the reaction mixture was diluted with 20 ml of water, acidified with 1M hydrochloric acid and extracted twice with 40 ml each time of ethyl acetate. The collected organic phases were washed once each with 20 ml of water and saturated aqueous sodium chloride solution, dried over magnesium sulphate and filtered, and the filtrate was concentrated. The residue was stirred with methyl tert-butyl ether/ethyl acetate (1:1), and the solid was filtered off, dried and separated by means of preparative HPLC (eluent: acetonitrile/water with 0.1% formic acid (gradient)). The product-containing fractions were partly concentrated and the precipitated solid was filtered off and dried under high vacuum. This gave 270 mg of the title compound as a crude product, which was converted further without further purification.

LC-MS (Method 1): Rₜ=0.99 min; MS (ESIpos): m/z=696 [M+H]⁺.

### Example 202A

### tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2-chloro-3-methylbiphenyl-4-yl}carbonyl)amino]piperidine-1-carboxylate

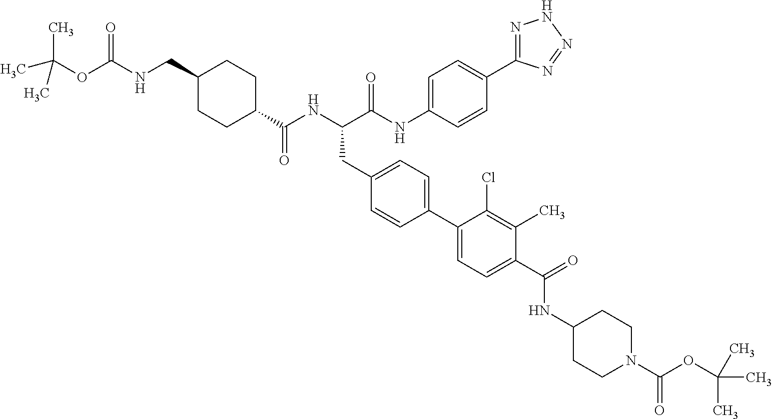 [SMILES: Cc1c(C(=O)NC2CCN(C(=O)OC(C)(C)C)CC2)ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1Cl]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-chloro-3-methylbiphenyl-4-carboxylic acid (175 mg, 0.24 mmol) was initially charged in 3.4 ml of DMF, and tert-butyl 4-aminopiperidine-1-carboxylate (98 mg, 0.49 mmol) and N,N-diisopropylethylamine (0.17 ml, 0.98 mmol) were added. Subsequently, 1-[bis(dimethylamino)methylene]-1H-1,2,3-triazolo[4,5-b]pyridinium 3-oxide hexafluorophosphate (140 mg, 0.37 mmol) was added and the mixture was stirred at RT for 1 h and left to stand overnight. The reaction mixture was diluted with 2.0 ml of acetonitrile and separated by means of preparative HPLC (eluent: acetonitrile/water with 0.1% formic acid (gradient)). The product-containing fractions were partly concentrated and the precipitate formed was filtered off and dried under high vacuum. This gave 120 mg (49% of theory) of the title compound.

LC-MS (Method 1): Rₜ=1.18 min; MS (ESIpos): m/z=899 [M+H]⁺.

¹H NMR (400 MHz, DMSO-d₆): δ=ppm 16.70 (br. s., 1H), 10.42 (s, 1H), 8.37 (d, 1H), 8.16 (d, 1H), 7.99 (d, 2H), 7.82 (d, 2H), 7.39 (d, 2H), 7.32-7.24 (m, 3H), 7.19 (d, 1H), 6.80-6.72 (m, 1H), 4.81-4.69 (m, 1H), 4.01-3.82 (m, 3H), 3.18-3.08 (m, 1H), 3.01-2.82 (m, 3H), 2.78-2.70 (m, 2H), 2.37 (s, 3H), 2.17-2.04 (m, 1H), 1.87-1.77 (m, 2H), 1.73-1.59 (m, 3H), 1.57-1.49 (m, 1H), 1.40 (s, 9H), 1.37 (s, 9H), 1.28-1.06 (m, 3H), 0.91-0.72 (m, 2H).

### Example 203A

### tert-Butyl [(trans-4-({[(2S)-3-{2′-chloro-3′-methyl-4′-[(1-methylpiperidin-4-yl)carbamoyl]biphenyl-4-yl}-1-oxo-1-{[4-(2H-tetrazol-5-yl)phenyl]amino}propan-2-yl]carbamoyl}cyclohexyl)-methyl]carbamate

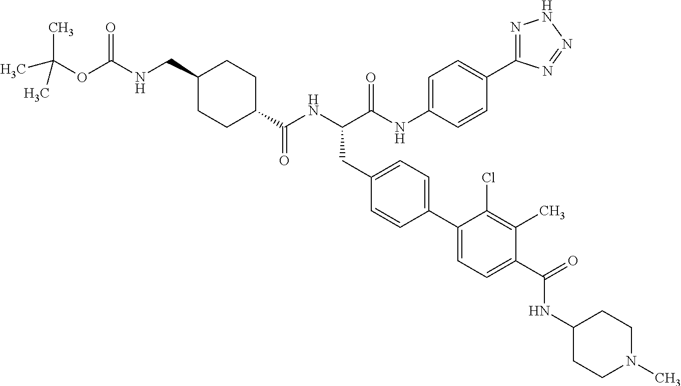 [SMILES: Cc1c(C(=O)NC2CCN(C)CC2)ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1Cl]

4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-chloro-3-methylbiphenyl-4-carboxylic acid (55 mg, 0.08 mmol) was initially charged in 1.0 ml of DMF, and 1-methylpiperidin-4-amine (18 mg, 0.15 mmol) and N,N-diisopropylethylamine (0.05 ml, 0.31 mmol) were added. Subsequently, 1-[bis(dimethylamino)methylene]-1H-1,2,3-triazolo[4,5-b]pyridinium 3-oxide hexafluorophosphate (44 mg, 0.12 mmol) was added and the mixture was stirred at RT for 6 h and left to stand for two days. The reaction mixture was diluted with 2.0 ml of acetonitrile and separated by means of preparative HPLC (eluent: acetonitrile/water with 0.1% formic acid (gradient)). This gave 31 mg (49% of theory) of the title compound.

LC-MS (Method 1): Rₜ=0.80 min; MS (ESIpos): m/z=813 [M+H]⁺.

¹H NMR (400 MHz, DMSO-d₆): δ=ppm 10.23 (s, 1H), 8.48 (d, 1H), 8.17-8.09 (m, 1H), 7.94 (d, 2H), 7.69 (d, 2H), 7.38 (d, 2H), 7.31-7.25 (m, 3H), 7.21 (d, 1H), 6.79-6.72 (m, 1H), 4.79-4.70 (m, 1H), 4.00-3.89 (m, 1H), 3.15-3.11 (m, 1H), 2.98-2.91 (m, 1H), 2.89-2.80 (m, 1H), 2.78-2.72 (m, 1H), 2.38 (s, 3H), 2.15-2.07 (m, 1H), 2.03-1.94 (m, 2H), 1.75-1.59 (m, 5H), 1.58-1.52 (m, 1H), 1.37 (s, 9H), 1.30-1.19 (m, 3H), 1.19-1.08 (m, 1H), 0.88-0.76 (m, 2H).

### Example 204A

### 4′-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]-2-chloro-3-methylbiphenyl-4-carboxylic acid

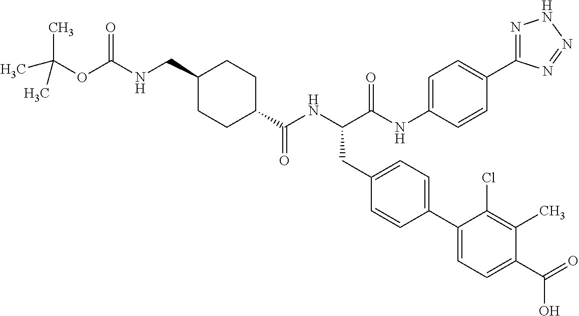 [SMILES: Cc1c(C(=O)O)ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)c1Cl]

3-Chloro-4-(dihydroxyboryl)-2-methylbenzoic acid (48 mg, 0.22 mmol), 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (100 mg, 0.16 mmol) from Example 4A and 51 mg (0.48 mmol) of sodium carbonate were initially charged in a mixture of 1.0 ml of DMF and 0.2 ml of water under argon. Subsequently, 1,1-bis(diphenylphosphino)ferrocenedichloropalladium(II) (12 mg, 0.02 mmol) was added and the mixture was stirred at 140° C. in a microwave for 1 h. Thereafter, the reaction mixture was diluted with 10 ml of water, acidified with 1M hydrochloric acid and extracted twice with 20 ml each time of ethyl acetate. The collected organic phases were washed once each with water and saturated aqueous sodium chloride solution, dried over magnesium sulphate and filtered, and the filtrate was concentrated. The residue was stirred with acetonitrile, and the solid was filtered off, washed with acetonitrile and dried under high vacuum. This gave 60 mg of the title compound as a crude product, which was converted further without further purification.

LC-MS (Method 1): Rₜ=1.00 min; MS (ESIpos): m/z=714 [M−H]⁻.

### Example 205A

### tert-Butyl 4-{[(4′-{(2S)-2-{[(trans-4-{[(tert-butoxycarbonyl)amino]methyl}cyclohexyl)carbonyl]-amino}-3-[(4-{3-[3-(dimethylamino)-1,1,2,2-tetrafluoro-3-oxopropyl]-1H-1,2,4-triazol-5-yl}phenyl)amino]-3-oxopropyl}-2-methylbiphenyl-4-yl)carbonyl]amino}piperidine-1-carboxylate

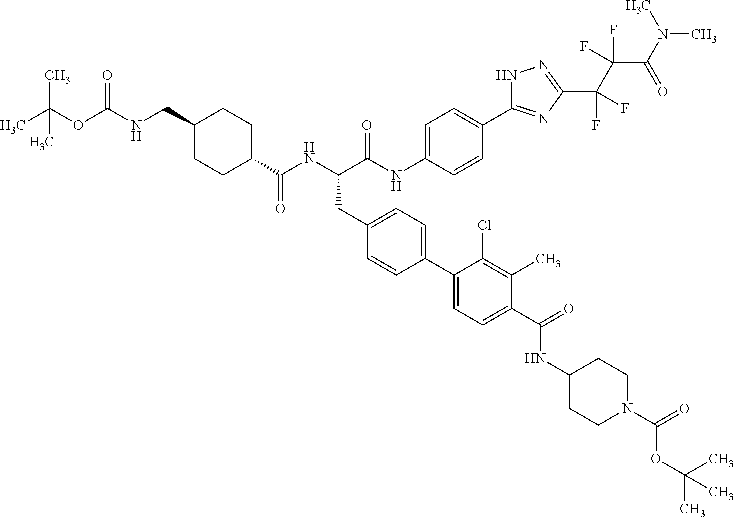 [SMILES: Cc1c(C(=O)NC2CCN(C(=O)OC(C)(C)C)CC2)ccc(-c2ccc(C[C@H](NC(=O)[C@H]3CC[C@H](CNC(=O)OC(C)(C)C)CC3)C(=O)Nc3ccc(-c4nc(C(F)(F)C(F)(F)C(=O)N(C)C)n[nH]4)cc3)cc2)c1Cl]

(2S)-2-{[(trans-4-{[(tert-Butoxycarbonyl)amino]methyl}cyclohexyl)carbonyl]amino}-3-(4′-{[1-(tert-butoxycarbonyl)piperidin-4-yl]carbamoyl}-2′-methylbiphenyl-4-yl)propanoic acid (100 mg, 0.14 mmol) and 3-[5-(4-aminophenyl)-1H-1,2,4-triazol-3-yl]-2,2,3,3-tetrafluoro-N,N-dimethylpropanamide (55 mg, 0.17 mmol) were dissolved in 0.5 ml of pyridine and admixed with 2,4,6-tripropyl-1,3,5,2,4,6-trioxatriphosphinane 2,4,6-trioxide (50% in ethyl acetate, 0.33 ml, 0.56 mmol) and stirred at RT for 18 h. The solvent was removed and the residue was admixed with dilute aqueous ammonium chloride solution and extracted repeatedly with ethyl acetate. The organic phases were washed with saturated aqueous sodium chloride solution, dried over sodium sulphate and concentrated under reduced pressure. The residue was dissolved, filtered through a Millipore filter and purified via preparative HPLC (eluent: acetonitrile/water with 0.1% trifluoroacetic acid (gradient)). This gave 83 mg (58% of theory) of the title compound.

LC-MS (Method 1): Rₜ=1.18 min; MS (ESIpos): m/z=1034.6 [M+H]⁺.

### Example 206A

### tert-Butyl 4-[({4′-[(2S)-3-({4-[3-(3-amino-1,1,2,2-tetrafluoro-3-oxopropyl)-1H-1,2,4-triazol-5-yl]phenyl}amino)-2-{[(trans-4-{[(tert-butoxycarbonyl)amino]methyl}cyclohexyl)carbonyl]-amino}-3-oxopropyl]-2-methylbiphenyl-4-yl}carbonyl)amino]piperidine-1-carboxylate

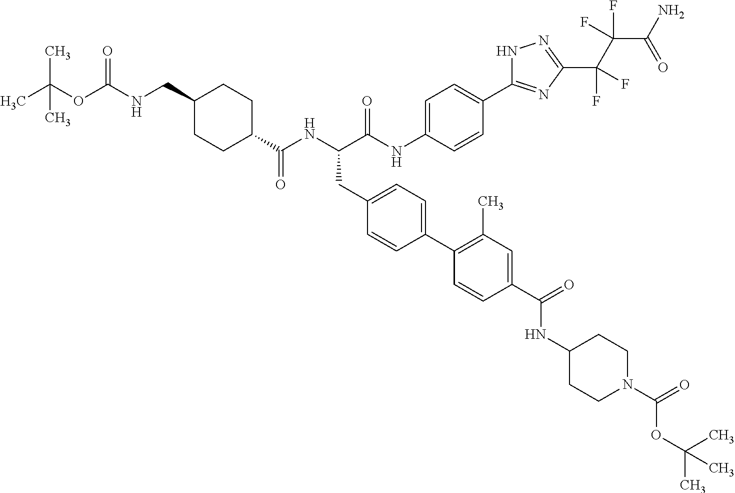 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc(-c3nc(C(F)(F)C(F)(F)C(N)=O)n[nH]3)cc2)cc1]

(2S)-2-{[(trans-4-{[(tert-Butoxycarbonyl)amino]methyl}cyclohexyl)carbonyl]amino}-3-(4′-{[1-(tert-butoxycarbonyl)piperidin-4-yl]carbamoyl}-2′-methylbiphenyl-4-yl)propanoic acid (100 mg, 0.14 mmol) and 3-[5-(4-aminophenyl)-1H-1,2,4-triazol-3-yl]-2,2,3,3-tetrafluoropropanamide (50 mg, 0.17 mmol) were dissolved in 0.5 ml of pyridine and admixed with 2,4,6-tripropyl-1,3,5,2,4,6-trioxatriphosphinane 2,4,6-trioxide (50% in ethyl acetate, 0.33 ml, 0.56 mmol) and stirred at RT for 18 h. The solvent was removed and the residue was admixed with dilute aqueous ammonium chloride solution and extracted repeatedly with ethyl acetate. The organic phases were washed with saturated aqueous sodium chloride solution, dried over sodium sulphate and concentrated under reduced pressure. This gave 24 mg (15% of theory) of the title compound.

LC-MS (Method 1): Rₜ=1.12 min; MS (ESIpos): m/z=1006.2 [M+H]⁺.

### Example 207A

### tert-Butyl 4-{[(4′-{(2S)-2-{[(trans-4-{[(tert-butoxycarbonyl)amino]methyl}cyclohexyl)carbonyl]-amino}-3-oxo-3-[(4-{3-[1,1,2,2-tetrafluoro-3-(methylamino)-3-oxopropyl]-1H-1,2,4-triazol-5-yl}phenyl)amino]propyl}-2-methylbiphenyl-4-yl)carbonyl]amino}piperidine-1-carboxylate

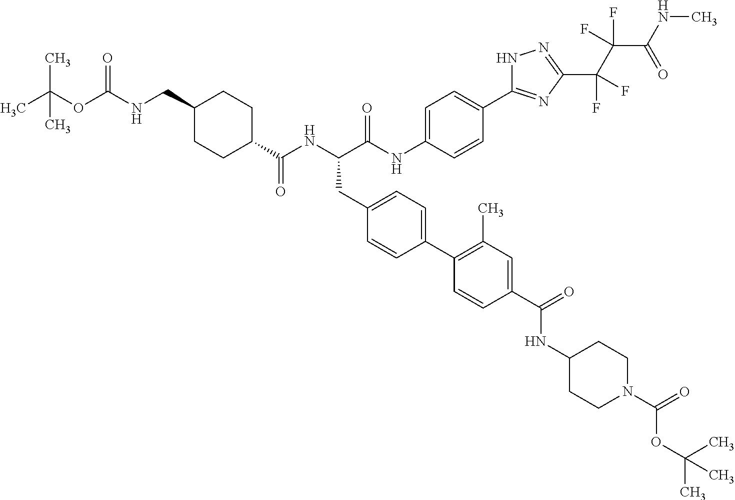 [SMILES: CNC(=O)C(F)(F)C(F)(F)c1n[nH]c(-c2ccc(NC(=O)[C@H](Cc3ccc(-c4ccc(C(=O)NC5CCN(C(=O)OC(C)(C)C)CC5)cc4C)cc3)NC(=O)[C@H]3CC[C@H](CNC(=O)OC(C)(C)C)CC3)cc2)n1]

(2S)-2-{[(trans-4-{[(tert-Butoxycarbonyl)amino]methyl}cyclohexyl)carbonyl]amino}-3-(4′-{[1-(tert-butoxycarbonyl)piperidin-4-yl]carbamoyl}-2′-methylbiphenyl-4-yl)propanoic acid (100 mg, 0.14 mmol) and 3-[5-(4-aminophenyl)-1H-1,2,4-triazol-3-yl]-2,2,3,3-tetrafluoro-N-methylpropanamide (53 mg, 0.17 mmol) were dissolved in 0.5 ml of pyridine and admixed with 2,4,6-tripropyl-1,3,5,2,4,6-trioxatriphosphinane 2,4,6-trioxide (50% in ethyl acetate, 0.33 ml, 0.56 mmol) and stirred at RT for 18 h. The solvent was removed and the residue was admixed with dilute aqueous ammonium chloride solution and extracted repeatedly with ethyl acetate. The organic phases were washed with saturated aqueous sodium chloride solution, and dried over sodium sulphate and under reduced pressure. This gave 42 mg (27% of theory) of the title compound.

LC-MS (Method 1): Rₜ=1.14 min; MS (ESIpos): m/z=1020.6 [M+H]1.

### Example 208A

### tert-Butyl 4-[({4′-[(2S)-2-{[(trans-4-{[(tert-butoxycarbonyl)amino]methyl}cyclohexyl)carbonyl]-amino}-3-oxo-3-({4-[3-(1,1,2,2-tetrafluoro-3-methoxy-3-oxopropyl)-1H-1,2,4-triazol-5-yl]phenyl}amino)propyl]-2-methylbiphenyl-4-yl}carbonyl)amino]piperidine-1-carboxylate

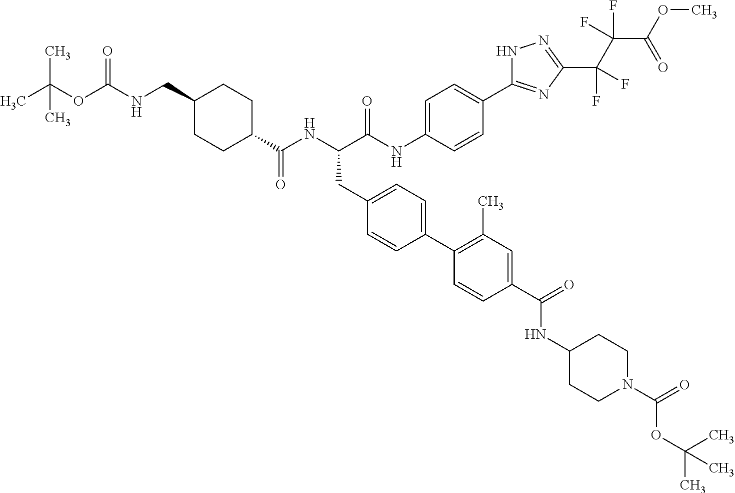 [SMILES: COC(=O)C(F)(F)C(F)(F)c1n[nH]c(-c2ccc(NC(=O)[C@H](Cc3ccc(-c4ccc(C(=O)NC5CCN(C(=O)OC(C)(C)C)CC5)cc4C)cc3)NC(=O)[C@H]3CC[C@H](CNC(=O)OC(C)(C)C)CC3)cc2)n1]

(2S)-2-{[(trans-4-{[(tert-Butoxycarbonyl)amino]methyl}cyclohexyl)carbonyl]amino}-3-(4′-{[1-(tert-butoxycarbonyl)piperidin-4-yl]carbamoyl}-2′-methylbiphenyl-4-yl)propanoic acid (100 mg, 0.14 mmol) and methyl 3-[5-(4-aminophenyl)-1H-1,2,4-triazol-3-yl]-2,2,3,3-tetrafluoropropanoate (53 mg, 0.17 mmol) were dissolved in 1 ml of pyridine and admixed with 2,4,6-tripropyl-1,3,5,2,4,6-trioxatriphosphinane 2,4,6-trioxide (50% in dimethylformamide, 0.33 ml, 0.56 mmol) and stirred at 85° C. for 15 min. The reaction mixture was filtered through a Millipore filter and purified via preparative HPLC (eluent: acetonitrile/water with 0.1% trifluoroacetic acid (gradient)). This gave 20 mg (13% of theory) of the title compound.

LC-MS (Method 17): Rₜ=1.56 min; MS (ESIpos): m/z=1021.5 [M+H]⁺.

### Example 209A

### Methyl 2,2,3,3-tetrafluoro-3-[5-(4-nitrophenyl)-1H-1,2,4-triazol-3-yl]propanoate

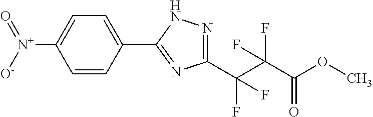 [SMILES: COC(=O)C(F)(F)C(F)(F)c1n[nH]c(-c2ccc([N+](=O)[O-])cc2)n1]

2,2,3,3-Tetrafluoro-3-[5-(4-nitrophenyl)-1H-1,2,4-triazol-3-yl]propanoic acid (30.3 g, 90.8 mmol) was dissolved in methanol (500 ml) and admixed with concentrated sulphuric acid (3 ml). The mixture was stirred at 65° C. for 22 h. Then concentrated sulphuric acid (5 ml) was added and the mixture was stirred once again at 65° C. for 22 h. Sodium hydrogencarbonate was added at RT to pH=7, the mixture was filtered and the solvent was removed under reduced pressure. The residue was stirred in petroleum ether and diethyl ether and then filtered. This gave 31.6 g (77% of theory) of the title compound.

LC-MS (Method 1): Rₜ=0.96 min; MS (ESIpos): m/z=349.1 [M+H]⁺.

### Example 210A

### Methyl 3-[5-(4-aminophenyl)-1H-1,2,4-triazol-3-yl]-2,2,3,3-tetrafluoropropanoate

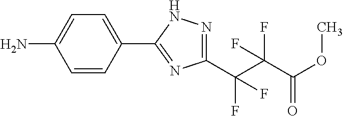 [SMILES: COC(=O)C(F)(F)C(F)(F)c1n[nH]c(-c2ccc(N)cc2)n1]

Methyl 2,2,3,3-tetrafluoro-3-[5-(4-nitrophenyl)-1H-1,2,4-triazol-3-yl]propanoate (24.0 g, 68.9 mmol) was initially charged in THF (370 ml) and admixed with palladium/charcoal (10%, 50% water-moist) under an argon atmosphere. Hydrogenation was effected with hydrogen (1 bar) at RT for 18 h. The mixture was filtered through kieselguhr and washed with dichloromethane/methanol 9:1. The filtrate was concentrated and the residue was dried under reduced pressure. This gave 21.7 g (99% of theory) of the title compound.

LC-MS (Method 1): Rₜ=0.78 min; MS (ESIpos): m/z=319.1 [M+H]⁺.

### Example 211A

### 3-[5-(4-Aminophenyl)-1H-1,2,4-triazol-3-yl]-2,2,3,3-tetrafluoro-N,N-dimethylpropanamide

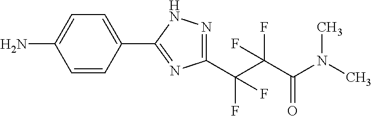 [SMILES: CN(C)C(=O)C(F)(F)C(F)(F)c1n[nH]c(-c2ccc(N)cc2)n1]

Methyl 3-[5-(4-aminophenyl)-1H-1,2,4-triazol-3-yl]-2,2,3,3-tetrafluoropropanoate (1.0 g, 3.1 mmol) was initially charged in THF (10 ml) and admixed with dimethylamine (2.4 ml, 4.7 mmol) under an argon atmosphere. The mixture was stirred at RT for 18 h. Dimethylamine (2.4 ml, 4.7 mmol) was added once again and the mixture was stirred at RT for 18 h. The solvent was removed and the residue was dissolved, filtered through a Millipore filter and purified via preparative HPLC (eluent: acetonitrile/water with 0.1% trifluoroacetic acid (gradient)). This gave 728 mg (68% of theory) of the title compound.

LC-MS (Method 1): Rₜ=0.68 min; MS (ESIpos): m/z=332.1 [M+H]⁺.

### Example 212A

### 3-[5-(4-Aminophenyl)-1H-1,2,4-triazol-3-yl]-2,2,3,3-tetrafluoropropanamide

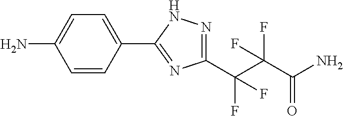 [SMILES: NC(=O)C(F)(F)C(F)(F)c1n[nH]c(-c2ccc(N)cc2)n1]

Methyl 3-[5-(4-aminophenyl)-1H-1,2,4-triazol-3-yl]-2,2,3,3-tetrafluoropropanoate (1.0 g, 3.1 mmol) was initially charged in THF (10 ml) and admixed with ammonia (1M in ethanol, 2.4 ml, 4.7 mmol) under an argon atmosphere. The mixture was stirred at RT for 18 h. Ammonia (1M in ethanol, 2.4 ml, 4.7 mmol) was added once again and the mixture was stirred at RT for 18 h. The solvent was removed and the residue was repeatedly coevaporated with acetonitrile. Then the residue was admixed with a little water/acetonitrile. The solid was filtered off with suction and dried under high vacuum. This gave 549 mg (56% of theory) of the title compound.

LC-MS (Method 1): Rₜ=0.51 min; MS (ESIpos): m/z=304.1 [M+H]⁺.

### Example 213A

### 3-[5-(4-Aminophenyl)-1H-1,2,4-triazol-3-yl]-2,2,3,3-tetrafluoro-N-methylpropanamide

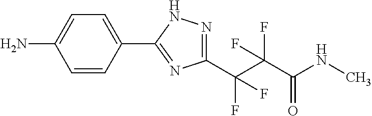 [SMILES: CNC(=O)C(F)(F)C(F)(F)c1n[nH]c(-c2ccc(N)cc2)n1]

Methyl 3-[5-(4-aminophenyl)-1H-1,2,4-triazol-3-yl]-2,2,3,3-tetrafluoropropanoate (1.0 g, 3.1 mmol) was initially charged in THF (10 ml) and admixed with methylamine (2M in tetrahydrofuran, 2.4 ml, 4.7 mmol) under an argon atmosphere. The mixture was stirred at RT for 18 h. Methylamine (2M in tetrahydrofuran, 2.4 ml, 4.7 mmol) was added once again and the mixture was stirred at RT for 18 h. The solvent was removed and the residue was admixed with a little water/acetonitrile. The solid was filtered off with suction and dried under high vacuum. This gave 412 mg (40% of theory) of the title compound.

LC-MS (Method 1): Rₜ=0.55 min; MS (ESIpos): m/z=318.1 [M+H]⁺.

### Example 214A

### 2,2,3,3-Tetrafluoro-3-[5-(4-nitrophenyl)-1H-1,2,4-triazol-3-yl]propanoic acid

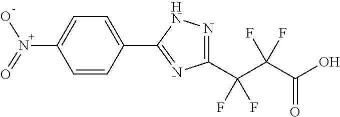

4-Nitrobenzenecarboximidohydrazide (1.22 g, 6.8 mmol) was dissolved in 50 ml of dichloromethane and admixed with 3,3,4,4-tetrafluorodihydrofuran-2,5-dione (3.5 g, 20.3 mmol). The reaction mixture was stirred at RT for 2 min, admixed with 50 ml of acetonitrile and stirred at RT overnight. The reaction mixture was concentrated and converted further as the crude product.

LC-MS (Method 4): Rₜ=0.72 min; MS (ESIneg): m/z=333.1 [M−H]⁻.

### Example 215A

### 3-[5-(4-Aminophenyl)-1H-1,2,4-triazol-3-yl]-2,2,3,3-tetrafluoropropanoic acid

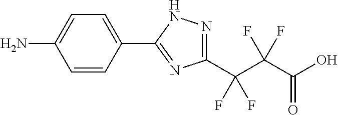 [SMILES: Nc1ccc(-c2nc(C(F)(F)C(F)(F)C(=O)O)n[nH]2)cc1]

2,2,3,3-Tetrafluoro-3-[5-(4-nitrophenyl)-1H-1,2,4-triazol-3-yl]propanoic acid (2.3 g, 69 mmol) was dissolved in 115 ml of methanol, admixed with ammonium formate (1.74 g, 27.5 mmol) and palladium/charcoal (10%, 732 mg, 0.7 mmol) and stirred at RT for 30 min. The reaction mixture was filtered and concentrated and converted further as the crude product.

LC-MS (Method 4): Rₜ=0.45 min; MS (ESIpos): m/z=305.0 [M+H]⁺.

### Example 216A

### 3-{5-[4-({4-Bromo-N-[(trans-4-{[(tert-butoxycarbonyl)amino]methyl}cyclohexyl)carbonyl]-L-phenylalanyl}amino)phenyl]-1H-1,2,4-triazol-3-yl}-2,2,3,3-tetrafluoropropanoic acid

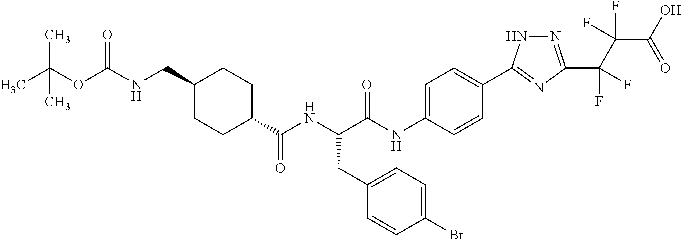

A solution of 4-bromo-N-[(trans-4-{[(tert-butoxycarbonyl)amino]methyl}-cyclohexyl)-carbonyl]-L-phenylalanine (1 g, 2.1 mmol) and 3-[5-(4-aminophenyl)-1H-1,2,4-triazol-3-yl]-2,2,3,3-tetrafluoropropanoic acid (1.38 g, 23 mmol, 50% purity) in ethyl acetate (125 ml) was admixed with N,N-diisopropylethylamine (1.1 ml, 6.2 mmol). Subsequently, 2,4,6-tripropyl-1,3,5,2,4,6-trioxatriphosphinane 2,4,6-trioxide solution (50% in ethyl acetate, 3.66 ml, 6.2 mmol) was added and the mixture was refluxed for 3 h. The reaction mixture was admixed with water, the phases were separated and the organic phase was washed with saturated aqueous sodium chloride solution, dried over sodium sulphate, filtered and concentrated. This gave 1.74 g (quant.) of the title compound.

LC-MS (Method 5): Rₜ=0.71 min; MS (ESIneg): m/z=767 [M−H]⁻.

### Example 217A

### 4-Bromo-N-[4-(dimethylamino)cyclohexyl]-3-(trifluoromethyl)benzamide

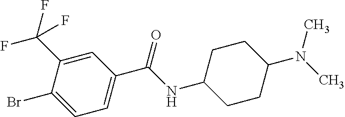 [SMILES: CN(C)C1CCC(NC(=O)c2ccc(Br)c(C(F)(F)F)c2)CC1]

A solution of 1.0 g (3.7 mmol) of 4-bromo-3-(trifluoromethyl)benzoic acid and 700 mg (3.7 mmol) of N,N-dimethylcyclohexane-1,4-diamine in 15 ml of dimethylformamide was admixed with 2.6 ml (14.8 mmol) of N,N-diisopropylethylamine and 2.8 g (7.4 mmol) of N-[(dimethylamino)(3H-[1,2,3]triazolo[4,5-b]pyridin-3-yloxy)methylidene]-N-methylmethanaminium hexafluorophosphate and stirred at RT overnight. The reaction mixture was concentrated and purified by chromatography via HPLC (2× Labomatic HD-3000 pump, Labomatic AS-3000, Knauer DAD 2600, Labomatic Labcol Vario 4000 Plus; Xbridge C18 5 μm 150 mm×50 mm; eluent A: water+0.2% ammonia solution (32%), eluent B: methanol; gradient: 0-12 min 50-90% B; flow rate: 150 ml/min). This gave 660 mg (45% of theory) of the title compound.

LC-MS (Method 4): Rₜ=0.90 min; MS (ESIpos): m/z=395.3 [M+H]⁺.

### Example 218A

### N-[4-(Dimethylamino)cyclohexyl]-4-(4,4,5,5-tetramethyl-1,3,2-dioxaborolan-2-yl)-3-(trifluoromethyl)benzamide

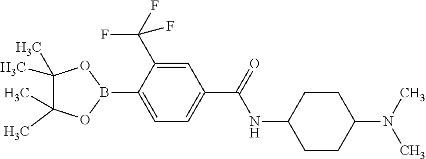

4-Bromo-N-[4-(dimethylamino)cyclohexyl]-3-(trifluoromethyl)benzamide (120 mg, 0.3 mmol) and bis(pinacolato)diborane (116 mg, 0.46 mmol) were dissolved in 1.5 ml of DMSO and admixed with 1,1′-bis(diphenylphosphino)ferrocenedichloropalladium(II) (12.5 mg, 0.015 mmol) and potassium acetate (90 mg, 0.9 mmol), and the mixture was stirred at 110° C. for 2 h and then converted further as the crude product.

LC-MS (Method 4): Rₜ=1.27 min; MS (ESIpos): m/z=662.5 [M+H]⁺.

### Example 219A

### 3-{5-[4-({(2S)-2-{[(trans-4-{[(tert-Butoxycarbonyl)amino]methyl}cyclohexyl)carbonyl]amino}-3-[4′-{[4-(dimethylamino)cyclohexyl]carbamoyl}-2′-(trifluoromethyl)biphenyl-4-yl]propanoyl}amino)phenyl]-1H-1,2,4-triazol-3-yl}-2,2,3,3-tetrafluoropropanoic acid

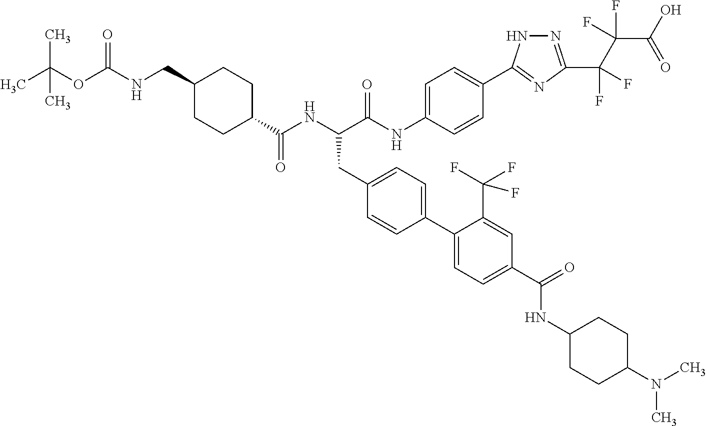

3-{5-[4-({4-Bromo-N-[(trans-4-{[(tert-butoxycarbonyl)amino]methyl}cyclohexyl)carbonyl]-L-phenylalanyl}amino)phenyl]-1H-1,2,4-triazol-3-yl}-2,2,3,3-tetrafluoropropanoic acid (150 mg, 0.19 mmol) and N-[4-(dimethylamino)cyclohexyl]-4-(4,4,5,5-tetramethyl-1,3,2-dioxaborolan-2-yl)-3-(trifluoromethyl)benzamide (128 mg, 0.3 mmol) were dissolved in 1.5 ml DMSO and admixed with tetrakis(triphenylphosphine)palladium (22 mg, 0.019 mmol), sodium carbonate (62 mg, 0.58 mmol) and water (0.3 ml). The reaction mixture was stirred at 110° C. in a microwave (Biotage Initiator) for 2 h. The reaction mixture was purified by chromatography via HPLC (Method 8). This gave 32 mg (17% of theory) of the title compound.

LC-MS (Method 4): Rₜ=0.99 min; MS (ESIpos): m/z=1003.9 [M+H]⁺.

### Example 220A

### tert-Butyl {[trans-4-({(2S)-3-(2′-methyl-4′-{[(2S)-1,1,1-trifluoropropan-2-yl]carbamoyl}biphenyl-4-yl)-1-oxo-1-[(2-oxo-2,3-dihydro-1H-benzimidazol-5-yl)amino]propan-2-yl}carbamoyl)cyclohexyl]methyl}carbamate

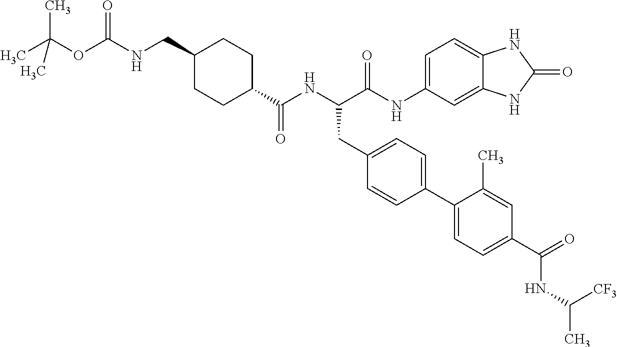

200 mg (0.30 mmol) of 4′-{(2S)-2-{[(trans-4-{[(tert-butoxycarbonyl)amino]methyl}cyclohexyl)-carbonyl]amino}-3-oxo-3-[(2-oxo-2,3-dihydro-1H-benzimidazol-5-yl)amino]propyl}-2-methylbiphenyl-4-carboxylic acid and 89 mg (0.6 mmol) of S-2,2,2-trifluoro-1-(methyl)ethylamine hydrochloride were dissolved in 3 ml of DMF and admixed with 227 mg (0.6 mmol) of N-[(dimethylamino)(3H-[1,2,3]triazolo[4,5-b]pyridin-3-yloxy)methylidene]-N-methylmethanaminium hexafluorophosphate and 0.2 ml (1.2 mmol) of N,N-diisopropylethylamine and the solution was stirred at RT overnight. The reaction mixture was admixed with water, and the precipitated solid was filtered off, dried under high vacuum and converted further as the crude product.

LC-MS (Method 4): Rₜ=1.23 min; MS (ESIpos): m/z=765.4 [M+H]⁺.

### Example 221A

### 4′-[(2S)-2-{[(trans-4-{[(tert-Butoxycarbonyl)amino]methyl}cyclohexyl)carbonyl]amino}-3-methoxy-3-oxopropyl]-2-chlorobiphenyl-4-carboxylic acid

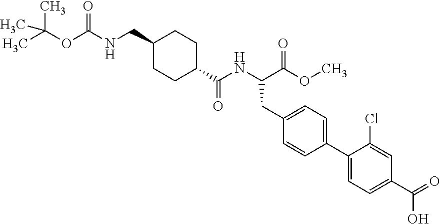

Methyl 4-bromo-N-[(trans-4-{[(tert-butoxycarbonyl)amino]methyl}cyclohexyl)carbonyl]-L-phenylalaninate (2.5 g, 5.0 mmol) and 2-chloro-4-carboxyphenylboronic acid (1.51 g, 7.5 mmol) were dissolved in 30 ml of dimethyl sulphoxide and admixed with tetrakis(triphenylphosphine)palladium(0) (578 mg, 0.5 mmol), sodium carbonate (1.6 g, 15.1 mmol) and water (7.6 ml, 0.42 mol). The reaction mixture was stirred at 110° C. in a microwave (Biotage Initiator) for 4 h, filtered, concentrated and purified by chromatography via HPLC (Method system: 2× Labomatic HD-3000 pump, Labomatic AS-3000, Knauer DAD 2600, Labomatic Labcol Vario 4000 Plus; column: Xbridge C18 5 μm 150 mm×50 mm; eluent A: water+0.1% formic acid (99%), eluent B: acetonitrile; gradient: 2.9-10 min 45-55% B; flow rate: 150 ml/min). This gave 2.0 g (69% of theory) of the title compound.

LC-MS (Method 4): Rₜ=1.23 min; MS (ESIneg): m/z=571.4 [M−H]⁻.

### Example 222A

### Methyl (2S)-2-{[(trans-4-{[(tert-butoxycarbonyl)amino]methyl}cyclohexyl)carbonyl]amino}-3-[2′-chloro-4′-(isopropylcarbamoyl)biphenyl-4-yl]propanoate

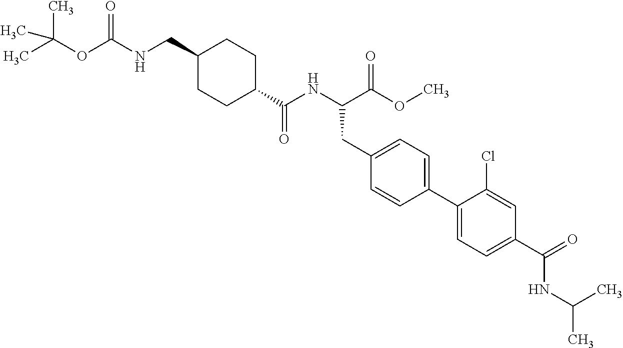

A solution of 750 mg (1.3 mmol) of 4′-[(2S)-2-{[(trans-4-{[(tert-butoxycarbonyl)amino]methyl}-cyclohexyl)carbonyl]amino}-3-methoxy-3-oxopropyl]-2-chlorobiphenyl-4-carboxylic acid and 234 mg (3.9 mmol) of isopropylamine in 12 ml of dimethylformamide was admixed with 0.9 ml (5.2 mmol) of N,N-diisopropylethylamine and 1.5 g (3.9 mmol) of N-[(dimethylamino)(3H-[1,2,3]triazolo[4,5-b]pyridin-3-yloxy)methylidene]-N-methylmethanaminium hexafluorophosphate and stirred at RT overnight. The reaction mixture was admixed with water, and the precipitate was filtered off and dried via lyophilization. This gave 643 mg (80% of theory) of the title compound.

LC-MS (Method 4): Rₜ=1.34 min; MS (ESIpos): m/z=614.4 [M+H]⁺.

### Example 223A

### (2S)-2-{[(trans-4-{[(tert-Butoxycarbonyl)amino]methyl}cyclohexyl)carbonyl]amino}-3-[2′-chloro-4′-(isopropylcarbamoyl)biphenyl-4-yl]propanoic acid

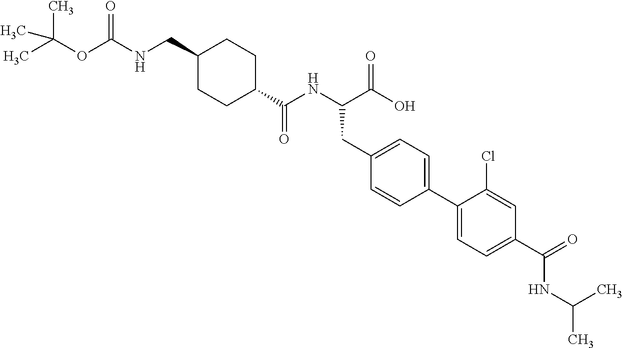

Methyl (2S)-2-{[(trans-4-{[(tert-butoxycarbonyl)amino]methyl}cyclohexyl)carbonyl]amino}-3-[2′-chloro-4′-(isopropylcarbamoyl)biphenyl-4-yl]propanoate (643 mg, 1.05 mmol) was dissolved in 15 ml of tetrahydrofuran, cooled to 0° C. and admixed with 1N sodium hydroxide solution (2.62 ml, 2.62 mmol). The reaction mixture was stirred at RT for 2 h and then the tetrahydrofuran was distilled off. The aqueous phase was washed with methyl tert-butyl ether and adjusted to pH 4, and the precipitated solid was filtered off with suction and dried under reduced pressure. This gave 523 mg (83% of theory) of the title compound.

LC-MS (Method 4): Rₜ=1.23 min; MS (ESIpos): m/z=600.5 [M+H]⁺.

### Example 224A

### tert-Butyl {[trans-4-({(2S)-3-[2′-chloro-4′-(isopropylcarbamoyl)biphenyl-4-yl]-1-[(4-fluoro-1H-indazol-6-yl)amino]-1-oxopropan-2-yl}carbamoyl)cyclohexyl]methyl}carbamate

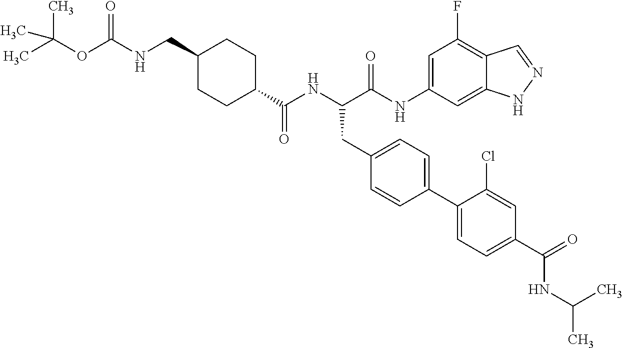

A solution of (2S)-2-{[(trans-4-{[(tert-butoxycarbonyl)amino]methyl}cyclohexyl)carbonyl]-amino}-3-[2′-chloro-4′-(isopropylcarbamoyl)biphenyl-4-yl]propanoic acid (100 mg, 0.17 mmol) and 4-fluoro-1H-indazol-6-amine (28 mg, 0.18 mmol) in ethyl acetate (2 ml) was admixed with N,N-diisopropylethylamine (0.07 ml, 0.5 mmol). Subsequently, 2,4,6-tripropyl-1,3,5,2,4,6-trioxatriphosphinane 2,4,6-trioxide solution (50% in ethyl acetate, 0.3 ml, 0.5 mmol) was added, and the mixture was refluxed for 1 h and stirred at RT overnight. The reaction mixture was admixed with water, and the precipitate was filtered off with suction and dried via lyophilization. This gave 20 mg (17% of theory) of the title compound.

LC-MS (Method 4): Rₜ=1.33 min; MS (ESIpos): m/z=733.5 [M+H]⁺.

### Example 225A

### tert-Butyl {[trans-4-({(2S)-3-[4′-(isopropylcarbamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate

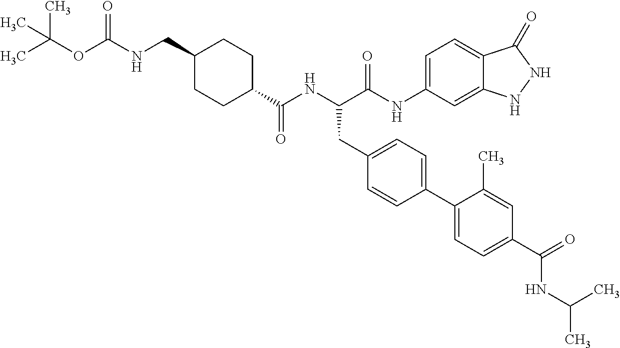

A solution of (2S)-2-{[(trans-4-{[(tert-butoxycarbonyl)amino]methyl}cyclohexyl)carbonyl]-amino}-3-[4′-(isopropylcarbamoyl)-2′-methylbiphenyl-4-yl]propanoic acid (150 mg, 0.26 mmol) and 6-amino-1,2-dihydro-3H-indazol-3-one (53 mg, 0.28 mmol) in ethyl acetate (3 ml) was admixed with N,N-diisopropylethylamine (0.11 ml, 0.77 mmol). Subsequently, 2,4,6-tripropyl-1,3,5,2,4,6-trioxatriphosphinane 2,4,6-trioxide solution (50% in ethyl acetate, 0.46 ml, 0.77 mmol) was added and the mixture was refluxed for 3 h. The reaction mixture was admixed with water, and the precipitate was filtered off with suction and dried via lyophilization. This gave 102 mg (55% of theory) of the title compound.

LC-MS (Method 4): Rₜ=1.16 min; MS (ESIpos): m/z=711.5 [M+H]⁺.

### Example 226A

### tert-Butyl [(trans-4-{[(2S)-3-{2′-chloro-4′-[(1-cyclopropylethyl)carbamoyl]biphenyl-4-yl}-1-oxo-1-{[4-(1H-tetrazol-5-yl)phenyl]amino}propan-2-yl]carbamoyl}cyclohexyl)methyl]carbamate

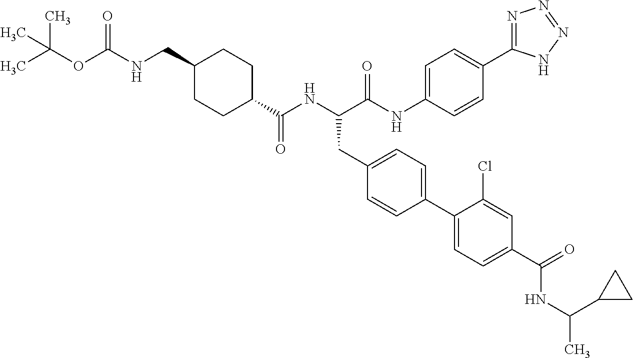

A solution of 150 mg (0.21 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-chlorobiphenyl-4-carboxylic acid and 37 mg (0.43 mmol) of 1-cyclopropylethanamine in 2 ml of dimethylformamide was admixed with 0.15 ml (0.85 mmol) of N,N-diisopropylethylamine and 244 mg (0.64 mmol) of N-[(dimethylamino)(3H-[1,2,3]triazolo[4,5-b]pyridin-3-yloxy)methylidene]-N-methylmethanaminium hexafluorophosphate and stirred at RT overnight. The reaction mixture was admixed with water, and the precipitate was filtered off and dried via lyophilization. This gave 140 mg (85% of theory) of the title compound.

LC-MS (Method 4): Rₜ=1.32 min; MS (ESIpos): m/z=769.5 [M+H]⁺.

### Example 227A

### 4′-{(2S)-2-{[(trans-4-{[(tert-Butoxycarbonyl)amino]methyl}cyclohexyl)carbonyl]amino}-3-oxo-3-[(2-oxo-2,3-dihydro-1H-benzimidazol-5-yl)amino]propyl}-2-chlorobiphenyl-4-carboxylic acid

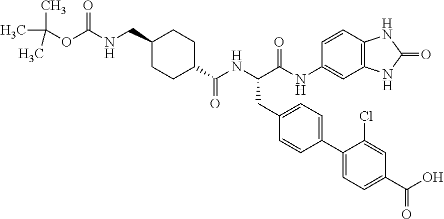

4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (1.0 g, 1.63 mmol) and 2-chloro-4-carboxyphenylboronic acid (504 mg, 2.44 mmol) were dissolved in 13 ml of dimethyl sulphoxide and admixed with tetrakis(triphenylphosphine)palladium(0) (188 mg, 0.16 mmol), sodium carbonate (517 mg, 4.9 mmol) and water (2.4 ml, 136 mmol). The reaction mixture was stirred at 110° C. in a microwave (Biotage Initiator) for 90 min, filtered, concentrated and purified by chromatography via HPLC (Method system: 2× Labomatic HD-3000 pump, Labomatic AS-3000, Knauer DAD 2600, Labomatic Labcol Vario 4000 Plus; column: Xbridge C18 5 μm 150 mm×50 mm; eluent A: water+0.2% ammonia solution (32%), eluent B: acetonitrile; gradient: 2.5-7.8 min 21-27% B; flow rate: 150 ml/min). This gave 524 mg (47% of theory) of the title compound.

LC-MS (Method 4): Rₜ=1.09 min; MS (ESIpos): m/z=690.5 [M+H]⁺.

### Example 228A

### tert-Butyl {[trans-4-({(2S)-3-[2′-chloro-4′-(isopropylcarbamoyl)biphenyl-4-yl]-1-oxo-1-[(2-oxo-2,3-dihydro-1H-benzimidazol-5-yl)amino]propan-2-yl}carbamoyl)cyclohexyl]methyl}carbamate

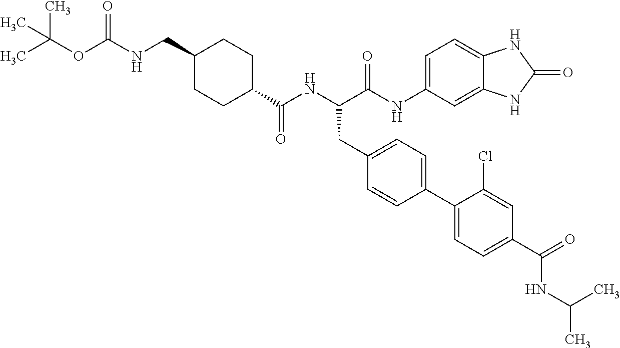

A solution of 150 mg (0.22 mmol) of 4′-{(2S)-2-{[(trans-4-{[(tert-butoxycarbonyl)amino]-methyl}cyclohexyl)carbonyl]amino}-3-oxo-3-[(2-oxo-2,3-dihydro-1H-benzimidazol-5-yl)-amino]propyl}-2-chlorobiphenyl-4-carboxylic acid and 39 mg (0.65 mmol) of isopropylamine in 2 ml of dimethylformamide was admixed with 0.15 ml (0.85 mmol) of N,N-diisopropylethylamine and 248 mg (0.65 mmol) of N-[(dimethylamino)(3H-[1,2,3]triazolo[4,5-b]pyridin-3-yloxy)methylidene]-N-methylmethanaminium hexafluorophosphate and stirred at RT for 48 h. The reaction mixture was admixed with water, and the precipitate was filtered off and dried via lyophilization. This gave 154 mg (97% of theory) of the title compound.

LC-MS (Method 4): Rₜ=1.21 min; MS (ESIpos): m/z=731.5 [M+H]⁺.

### Example 229A

### tert-Butyl [(trans-4-{[(2S)-3-[4′-(cyclobutylcarbamoyl)-2′-methylbiphenyl-4-yl]-1-oxo-1-{[4-(1H-tetrazol-5-yl)phenyl]amino}propan-2-yl]carbamoyl}cyclohexyl)methyl]carbamate

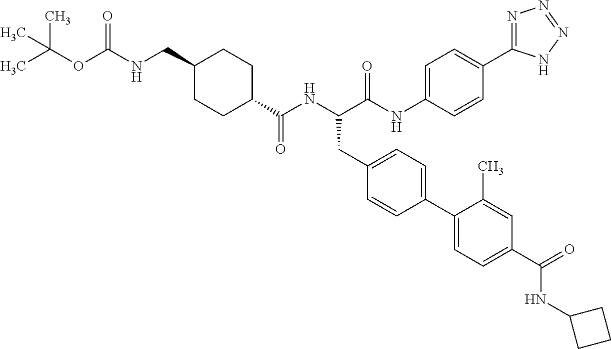

A solution of 250 mg (0.37 mmol) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 52.2 mg (0.73 mmol) of cyclobutanamine in 3.5 ml of dimethylformamide was admixed with 0.25 ml (1.5 mmol) of N,N-diisopropylethylamine and 418 mg (1.1 mmol) of N-[(dimethylamino)(3H-[1,2,3]triazolo[4,5-b]pyridin-3-yloxy)methylidene]-N-methylmethanaminium hexafluorophosphate and stirred at RT for 48 h. The reaction mixture was filtered through a Millipore filter and purified by chromatography via HPLC (Method 11). This gave 32 mg (12% of theory) of the title compound.

LC-MS (Method 4): Rₜ=1.25 min; MS (ESIpos): m/z=735.7 [M+H]⁺.

### Example 230A

### tert-Butyl [(trans-4-{[(2S)-3-{4′-[(1-hydroxypropan-2-yl)carbamoyl]-2′-methylbiphenyl-4-yl}-1-oxo-1-{[4-(1H-tetrazol-5-yl)phenyl]amino}propan-2-yl]carbamoyl}cyclohexyl)methyl]carbamate

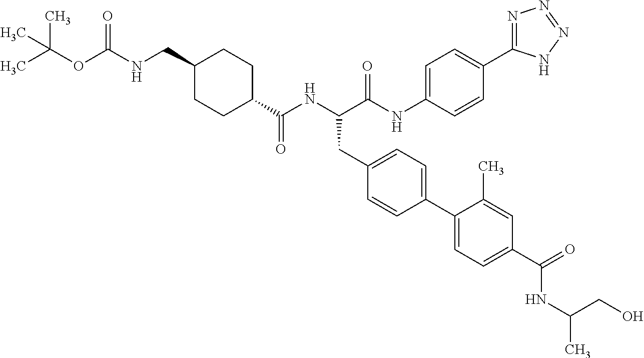

A solution of 200 mg (0.18 mmol, 60% purity) of 4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-carboxylic acid and 26 mg (0.35 mmol) of 2-aminopropan-1-ol in 2 ml of dimethylformamide was admixed with 0.09 ml (0.52 mmol) of N,N-diisopropylethylamine and 134 mg (0.35 mmol) of N-[(dimethylamino)(3H-[1,2,3]triazolo[4,5-b]pyridin-3-yloxy)methylidene]-N-methylmethanaminium hexafluorophosphate and stirred at RT overnight. The reaction mixture was filtered through a Millipore filter and purified by chromatography via HPLC (Method 11). This gave 30 mg (23% of theory) of the title compound.

LC-MS (Method 4): Rₜ=1.06 min; MS (ESIpos): m/z=739.8 [M+H]⁺.

### Example 231A

### tert-Butyl {[trans-4-({(2S)-3-[4′-(isopropylcarbamoyl)-2′-methylbiphenyl-4-yl]-1-[(1-methyl-2-oxo-2,3-dihydro-1H-benzimidazol-5-yl)amino]-1-oxopropan-2-yl}carbamoyl)cyclohexyl]-methyl}carbamate

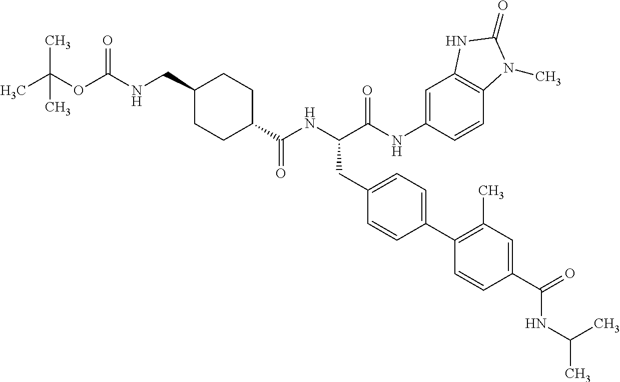 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2ccc3c(c2)[nH]c(=O)n3C)cc1]

A suspension of (2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoic acid (100 mg, 0.172 mmol) in ethyl acetate (2.5 ml) was admixed with 5-amino-1-methyl-1,3-dihydro-2H-benzimidazol-2-one (31 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 57.3 mg (45% of theory) of the title compound.

LC-MS (Method 1): Rₜ=1.01 min; MS (ESIneg): m/z=723 [M−H]⁻.

### Example 232A

### 4-Bromo-N-isopropyl-3-methylbenzamide

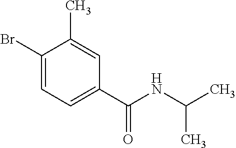

A solution of 4-bromo-3-methylbenzoic acid (25.45 g, 118.3 mmol) in DMF (255 ml) was admixed with N,N-diisopropylamine (3.6 ml, 20.5 mmol) and isopropylamine (11.2 ml, 130.2 mmol) and cooled to 0° C. Then HATU (54 g, 142 mmol) was added in portions. The reaction mixture was stirred at RT for 1 h. The mixture was added to water, then stirred for 10 min, and the precipitated solid was filtered off. The solid was washed twice with water, then dried under high vacuum. This gave 31.5 g (quant.) of the title compound with sufficient purity for further conversion.

LC-MS (Method 1): Rₜ=0.94 min; MS (ESIpos): m/z=256 [M+H]⁺.

### Example 233A

### Methyl (2S)-2-({[(trans-4-{[(tert-butoxycarbonyl)amino]methyl}cyclohexyl)carbonyl]amino}-3-[4′-(isopropylcarbamoyl)-2′-methylbiphenyl-4-yl]propanoate

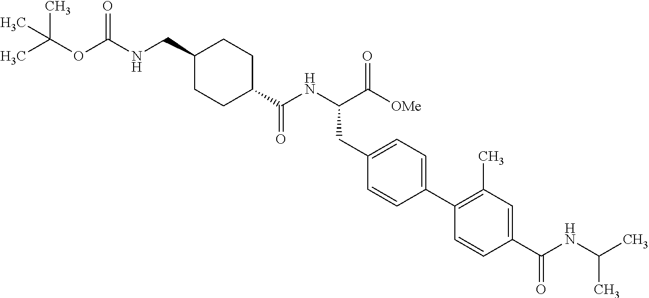 [SMILES: COC(=O)[C@H](Cc1ccc(-c2ccc(C(=O)NC(C)C)cc2C)cc1)NC(=O)[C@H]1CC[C@H](CNC(=O)OC(C)(C)C)CC1]

4-Bromo-N-isopropyl-3-methylbenzamide (26 g, 101.5 mmol) and bis(pinacolato)diboron (30.93 g, 121.8 mmol) were initially charged in 1,4-dioxane (520 ml) under argon, then admixed with potassium acetate (29.89 g, 304.5 mmol) and [1,1-bis(diphenylphosphino)ferrocene]dichloropalladium-dichloromethane complex (2.49 g, 3.05 mmol). The reaction mixture was stirred at 100° C. and the conversion was monitored by LC/MS. Subsequently, the mixture was cooled and admixed with methyl 4-bromo-N-[(trans-4-{[(tert-butoxycarbonyl)amino]methyl}cyclohexyl)carbonyl]-L-phenylalaninate (50.49 g, 101.5 mmol) and 2N aqueous sodium carbonate solution (150 ml), then stirred at 80-85° C. for 2.5 h. The suspension was cooled to RT and left to stand overnight. Thereafter, the mixture was filtered with suction through kieselguhr and washed with 1,4-dioxane. The filtrate was stirred into water and extracted with ethyl acetate. The combined organic phases were dried over sodium sulphate and filtered, and the solvent was removed on a rotary evaporator. The residue was dissolved in a little dichloromethane/methanol and purified by flash chromatography (eluent: ethyl acetate/cyclohexane (2:1), then dichloromethane/methanol (9:1 to 1:1)). The product-containing fractions were collected and the solvent was removed on a rotary evaporator. The residue was stirred in acetonitrile, then filtered with suction and washed copiously with acetonitrile. This gave 21.0 g (35% of theory) of the title compound. The wash solution was concentrated under reduced pressure. The residue thus obtained was stirred with acetonitrile, filtered off with suction and washed with a little acetonitrile. This gave an additional 21.1 g (35% of theory) of the title compound.

LC-MS (Method 13): Rₜ=3.48 min; MS (ESIpos): m/z=594 [M+H]⁺.

### Example 234A

### (2S)-2-{[(trans-4-{[(tert-Butoxycarbonyl)amino]methyl}cyclohexyl)carbonyl]amino}-3-[4′-(isopropylcarbamoyl)-2′-methylbiphenyl-4-yl]propanoic acid

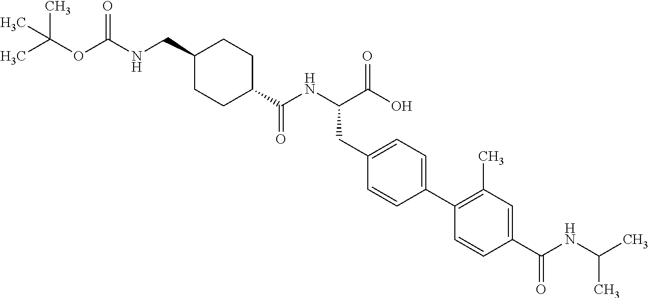 [SMILES: Cc1cc(C(=O)NC(C)C)ccc1-c1ccc(C[C@H](NC(=O)[C@H]2CC[C@H](CNC(=O)OC(C)(C)C)CC2)C(=O)O)cc1]

Methyl (2S)-2-{[(trans-4-{[(tert-butoxycarbonyl)amino]methyl}cyclohexyl)carbonyl]amino}-3-[4′-(isopropylcarbamoyl)-2′-methylbiphenyl-4-yl]propanoate (47.75 g, 80.42 mmol) was dissolved in tetrahydrofuran (750 ml), admixed with a solution of lithium hydroxide monohydrate (5.06 g, 120.63 mmol) in water (250 ml) and stirred at RT for 3 h. The mixture was added to water (1 l) and acidified slightly (pH 4-5) with 1N hydrochloric acid. Solid ammonium chloride was added to this solution, then the mixture was extracted with ethyl acetate (three times with 500 ml). The combined organic phases were washed with saturated aqueous sodium chloride solution, dried over sodium sulphate, filtered and concentrated under reduced pressure. Methyl tert-butyl ether was added to the residue and the mixture was stirred at 40° C. (water bath temperature) on a rotary evaporator for 10 min. The solid was filtered off with suction and washed with a mixture of diethyl ether/methyl tert-butyl ether (2:1), then dried under high vacuum overnight. The residue was dissolved in a little dichloromethane/methanol and applied to silica gel, then purified by flash chromatography (eluent: dichloromethane/methanol (10:1 to 5:1)). This gave 28.83 g (62% of theory) of the title compound.

¹H NMR (400 MHz, DMSO-d₆): δ=ppm 0.81 (m, 2H), 1.07-1.15 (m, 1H), 1.16 (s, 3H), 1.18 (s, 3H), 1.24 (m, 2H), 1.36 (s, 9H), 1.46-1.56 (m, 1H), 1.64 (m, 3H), 2.04 (m, 1H), 2.25 (s, 3H), 2.74 (m, 2H), 2.89 (m, 1H), 3.13 (dd, 1H), 4.11 (m, 1H), 4.38-4.51 (m, 1H), 6.76 (s, 1H), 7.19-7.27 (m, 3H), 7.27-7.33 (m, 2H), 7.70 (d, 1H), 7.75 (s, 1H), 7.95 (m, 1H), 8.18 (d, 1H), 12.72 (br. s, 1H).

LC-MS (Method 1): Rₜ=0.96 min; MS (ESIpos): m/z=580 [M+H]⁺.

### Example 235A

### 4-Bromo-N-cyclobutyl-3-methylbenzamide

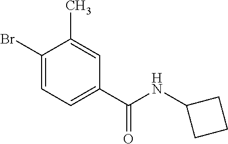

A solution of 4-bromo-3-methylbenzoic acid (2.0 g, 9.3 mmol) and cyclobutylamine (0.87 ml, 10.2 mmol) in DMF (60 ml) was admixed with N,N-diisopropylamine (3.6 ml, 20.5 mmol) and a solution of HATU (4.24 g, 11.2 mmol) in DMF (30 ml) was added thereto. The reaction mixture was stirred at RT overnight (about 16 h). The solvent was removed under high vacuum and the residue was admixed with water, stirred and filtered with suction. The residue was washed copiously with water and dried under high vacuum. This gave 2.35 g (94% of theory) of the title compound.

LC-MS (Method 2): Rₜ=2.15 min; MS (ESIpos): m/z=268 [M+H]⁺.

### Example 236A

### N-Cyclobutyl-3-methyl-4-(4,4,5,5-tetramethyl-1,3,2-dioxaborolan-2-yl)benzamide

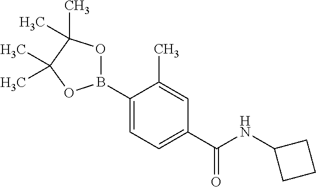 [SMILES: Cc1cc(C(=O)NC2CCC2)ccc1B1OC(C)(C)C(C)(C)O1]

A solution of 4-bromo-N-cyclobutyl-3-methylbenzamide (2.35 g, 8.76 mmol), bis(pinacolato)diborane (2.45 g, 9.64 mmol) and potassium acetate (1.72 g, 17.53 mmol) in toluene (52 ml) was degassed with argon and then admixed with [1,1-bis(diphenylphosphino)ferrocene]-dichloropalladium-dichloromethane complex (358 mg, 0.44 mmol). The mixture was then stirred at 110° C. for 6 h. The reaction mixture was concentrated on a rotary evaporator and dried under high vacuum. This gave 2.76 g (quant.) of the title compound, which was used further without purification.

LC-MS (Method 1): Rₜ=1.13 min; MS (ESIpos): m/z=316 [M+H]⁺.

### Example 237A

### (2S)-2-{[(trans-4-{[(tert-Butoxycarbonyl)amino]methyl}cyclohexyl)carbonyl]amino}-3-[4′-(cyclobutylcarbamoyl)-2′-methylbiphenyl-4-yl]propanoic acid

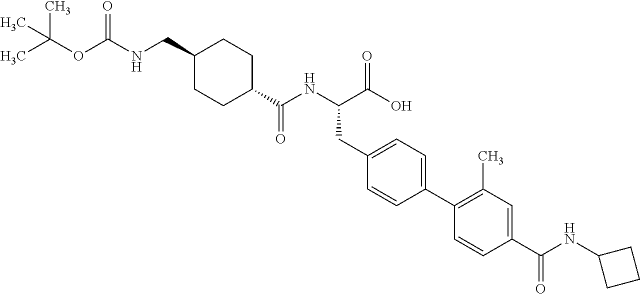 [SMILES: Cc1cc(C(=O)NC2CCC2)ccc1-c1ccc(C[C@H](NC(=O)[C@H]2CC[C@H](CNC(=O)OC(C)(C)C)CC2)C(=O)O)cc1]

To a solution of 4-bromo-N-[(trans-4-{[(tert-butoxycarbonyl)amino]methyl}cyclohexyl)-carbonyl]-L-phenylalanine (3.02 g, 6.25 mmol) and N-cyclobutyl-3-methyl-4-(4,4,5,5-tetramethyl-1,3,2-dioxaborolan-2-yl)benzamide (2.76 g, 8.76 mmol) in 1,2-dimethoxyethane (51 ml) were added ethanol (21 ml), 2N aqueous sodium carbonate solution (6.25 ml, 12.5 mmol) and [1,1-bis-(diphenylphosphino)ferrocene]dichloropalladium-dichloromethane complex (255.4 mg, 0.31 mmol). The mixture was then stirred at reflux (oil bath temperature 100° C.) for 8 h. The reaction mixture was concentrated on a rotary evaporator and the residue was dissolved in ethyl acetate. The ethyl acetate solution was washed twice with 10% aqueous citric acid solution, once with water, then washed with saturated aqueous sodium chloride solution. The organic phase was dried over sodium sulphate, then filtered and concentrated on a rotary evaporator. The residue was dissolved in DMSO/acetonitrile. The solution was filtered through a Millipore filter and purified by preparative HPLC (eluent: gradient of acetonitrile/water with 0.1% trifluoroacetic acid). This gave 2.21 g (60% of theory) of the title compound.

¹H NMR (400 MHz, DMSO-d₆): δ=ppm 0.68-0.90 (m, 2H), 1.23 (m, 3H), 1.36 (s, 9H), 1.46-1.55 (m, 1H), 1.57-1.75 (m, 5H), 1.98-2.13 (m, 3H), 2.15-2.24 (m, 2H), 2.26 (s, 3H), 2.74 (m, 2H), 2.84-2.96 (m, 1H), 3.07-3.19 (m, 1H), 4.33-4.58 (m, 2H), 6.70-6.86 (m, 1H), 7.16-7.36 (m, 5H), 7.66-7.72 (m, 1H), 7.76 (s, 1H), 8.04 (d, 1H), 8.59 (d, 1H), 12.68 (br. s, 1H).

LC-MS (Method 1): Rₜ=1.04 min; MS (ESIneg): m/z=590 [M−H]⁻.

### Example 238A

### Methyl 4′-[(2S)-3-(1H-benzotriazol-5-ylamino)-2-{[(trans-4-{[(tert-butoxycarbonyl)amino]-methyl}cyclohexyl)carbonyl]amino}-3-oxopropyl]-2-methylbiphenyl-4-carboxylate

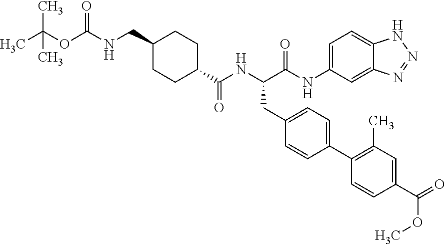 [SMILES: COC(=O)c1ccc(-c2ccc(C[C@H](NC(=O)[C@H]3CC[C@H](CNC(=O)OC(C)(C)C)CC3)C(=O)Nc3ccc4[nH]nnc4c3)cc2)c(C)c1]

A suspension of (2S)-2-{[(trans-4-{[(tert-butoxycarbonyl)amino]methyl}cyclohexyl)-carbonyl]amino}-3-[4′-(methoxycarbonyl)-2′-methylbiphenyl-4-yl]propanoic acid (1.5 g, 2.71 mmol) in ethyl acetate (40 ml) was admixed with tert-butyl 5-amino-1H-benzotriazole-1-carboxylate and tert-butyl 6-amino-1H-benzotriazole-1-carboxylate (699 mg, 2.99 mmol, regioisomer mixture (2:1)) and N,N-diisopropylethylamine (1.42 ml, 8.14 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was added to ethyl acetate and then washed twice with water and once with saturated aqueous sodium chloride solution. The organic phase was dried over sodium sulphate, then filtered and concentrated on a rotary evaporator. The residue was dissolved in DMSO/acetonitrile. The solution was filtered through a Millipore filter and purified by preparative HPLC (eluent: gradient of acetonitrile/water with 0.1% trifluoroacetic acid). This gave 236.4 mg (13% of theory) of the title compound and additionally 834.6 mg (36% of theory) of the title compound with the benzotriazole-Boc protecting group (tert-butyl 5-({(2S)-2-{[(trans-4-{[(tert-butoxycarbonyl)amino]methyl}cyclohexyl)carbonyl]amino}-3-[4′-(methoxycarbonyl)-2′-methylbiphenyl-4-yl]propanoyl}amino)-1H-benzotriazole-1-carboxylate and tert-butyl 6-({(2S)-2-{[(trans-4-{[(tert-butoxycarbonyl)amino]methyl}cyclohexyl)carbonyl]amino}-3-[4′-(methoxycarbonyl)-2′-methylbiphenyl-4-yl]propanoyl}amino)-1H-benzotriazole-1-carboxylate as a regioisomer mixture).

LC-MS (Method 1): Rₜ=1.09 min; MS (ESIneg): m/z=667 [M−H]⁻.

### Example 239A

### 4′-[(2S)-3-(1H-Benzotriazol-5-ylamino)-2-{[(trans-4-{[(tert-butoxycarbonyl)amino]methyl}-cyclohexyl)carbonyl]amino}-3-oxopropyl]-2-methylbiphenyl-4-carboxylic acid

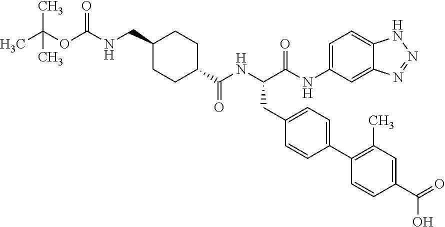 [SMILES: Cc1cc(C(=O)O)ccc1-c1ccc(C[C@H](NC(=O)[C@H]2CC[C@H](CNC(=O)OC(C)(C)C)CC2)C(=O)Nc2ccc3[nH]nnc3c2)cc1]

Methyl 4′-[(2S)-3-(1H-benzotriazol-5-ylamino)-2-{[(trans-4-{[(tert-butoxycarbonyl)amino]-methyl}cyclohexyl)carbonyl]amino}-3-oxopropyl]-2-methylbiphenyl-4-carboxylate (228 mg, 0.34 mmol) was dissolved in tetrahydrofuran/water 3/1 (8.6 ml), admixed with lithium hydroxide monohydrate (143.1 mg, 3.41 mmol) and stirred at RT overnight. The solution was diluted with ethyl acetate and adjusted to pH 5-6 with 1N hydrochloric acid. The phases were separated and the aqueous phase was extracted with ethyl acetate. The combined organic phases were washed with saturated aqueous sodium chloride solution, dried over sodium sulphate, filtered and concentrated under reduced pressure. This gave 137.8 mg (62% of theory) of the title compound.

LC-MS (Method 1): Rₜ=0.94 min; MS (ESIneg): m/z=653 [M−H]⁻.

### Example 240A

### tert-Butyl {[trans-4-({(2S)-1-(1H-benzotriazol-5-ylamino)-3-[4′-(cyclopropylcarbamoyl)-2′-methylbiphenyl-4-yl]-1-oxopropan-2-yl}carbamoyl)cyclohexyl]methyl}carbamate

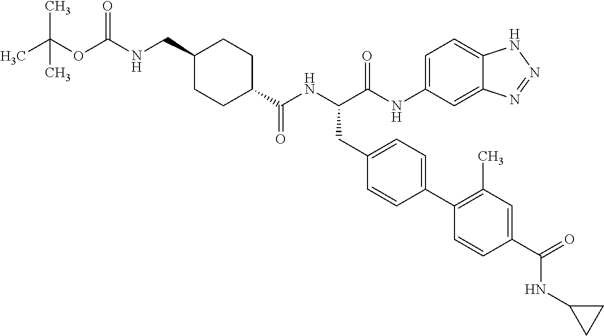 [SMILES: Cc1cc(C(=O)NC2CC2)ccc1-c1ccc(C[C@H](NC(=O)[C@H]2CC[C@H](CNC(=O)OC(C)(C)C)CC2)C(=O)Nc2ccc3[nH]nnc3c2)cc1]

A solution of 4′-[(2S)-3-(1H-benzotriazol-5-ylamino)-2-{[(trans-4-{[(tert-butoxycarbonyl)-amino]methyl}cyclohexyl)carbonyl]amino}-3-oxopropyl]-2-methylbiphenyl-4-carboxylic acid (70 mg, 0.11 mmol) and cyclopropylamine (0.015 ml, 0.21 mmol) in DMF (1 ml) was admixed with N,N-diisopropylamine (0.06 ml, 0.32 mmol), and HATU (61 mg, 0.16 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 49.4 mg (61% of theory) of the title compound.

LC-MS (Method 13): Rₜ=3.00 min; MS (ESIneg): m/z=692 [M−H]⁻.

### Example 241A

### tert-Butyl {[trans-4-({(2S)-1-(1H-benzimidazol-6-ylamino)-3-[4′-(isopropylcarbamoyl)-2′-methylbiphenyl-4-yl]-1-oxopropan-2-yl}carbamoyl)cyclohexyl]methyl}carbamate

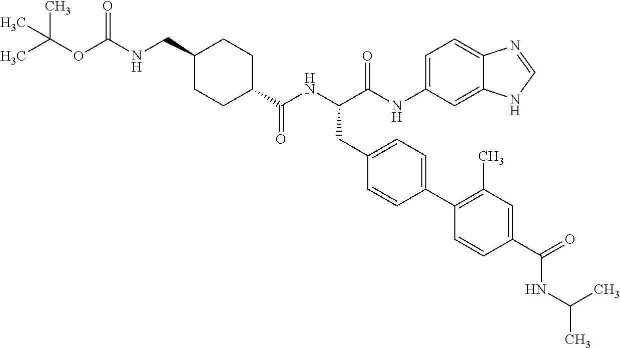 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2ccc3nc[nH]c3c2)cc1]

A suspension of (2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoic acid (100 mg, 0.172 mmol) in ethyl acetate (2.5 ml) was admixed with 1H-benzimidazol-6-amine (25.3 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 69.8 mg (48% of theory) of the title compound.

LC-MS (Method 1): Rₜ=0.86 min; MS (ESIneg): m/z=693 [M−H]⁻.

### Example 242A

### tert-Butyl [(trans-4-{[(2S)-1-(1H-indazol-6-ylamino)-3-{2′-methyl-4′-[(2-oxopiperidin-3-yl)carbamoyl]biphenyl-4-yl}-1-oxopropan-2-yl]carbamoyl}cyclohexyl)methyl]carbamate

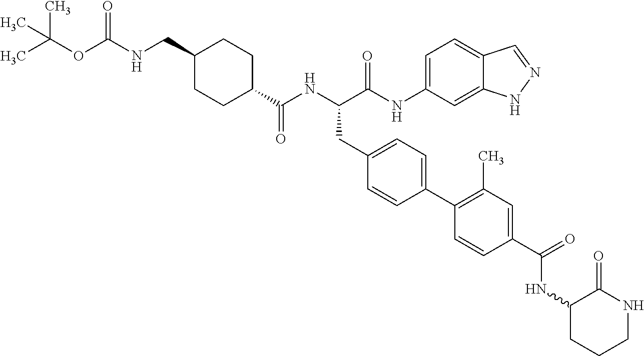 [SMILES: Cc1cc(C(=O)NC2CCCNC2=O)ccc1-c1ccc(C[C@H](NC(=O)[C@H]2CC[C@H](CNC(=O)OC(C)(C)C)CC2)C(=O)Nc2ccc3cn[nH]c3c2)cc1]

A suspension of 4′-[(2S)-2-{[(trans-4-{[(tert-butoxycarbonyl)amino]methyl}cyclohexyl)-carbonyl]amino}-3-(1H-indazol-6-ylamino)-3-oxopropyl]-2-methylbiphenyl-4-carboxylic acid (100 mg, 0.153 mmol) in ethyl acetate (2.5 ml) was admixed with 3-aminopiperidin-2-one (19.2 mg, 0.17 mmol) and N,N-diisopropylethylamine (0.08 ml, 0.46 mmol). The suspension was admixed with a 2,4,6-tripropyl-1,3,5,2,4,6-trioxatriphosphinane 2,4,6-trioxide solution (50% in DMF, 0.27 ml, 0.46 mmol) and then the mixture was stirred at reflux (oil bath temperature 80° C.) for 3 h. The reaction mixture was admixed with DMF (1 ml) and the ethyl acetate was removed on a rotary evaporator. The residue was admixed with a little water and acetonitrile, filtered through a Millipore filter and purified by preparative HPLC (eluent: gradient of acetonitrile/water with 0.1% trifluoroacetic acid). This gave 49.1 mg (42% of theory) of the title compound.

LC-MS (Method 1): Rₜ=0.92 min; MS (ESIneg): m/z=748 [M−H]⁻.

### Example 243A

### N⁴,N⁴,2,2-Tetramethylcyclohexane-1,4-diamine dihydrochloride

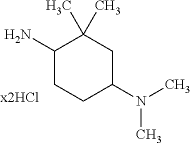

A solution of benzyl (2,2-dimethyl-4-oxocyclohexyl)carbamate (3.0 g, 10.9 mmol) in dichloromethane (20 ml) at 0° C. was admixed with a 2M dimethylamine solution in THF (10.9 ml, 21.8 mmol) and stirred at RT for 6 h. The reaction solution was cooled to 0° C. and admixed with sodium triacetoxyborhydride (3.46 g, 16.4 mmol) in small portions. Then the mixture was stirred at RT for 6 h. Cold water was added thereto and the mixture was extracted with dichloromethane (three times with 250 ml). The combined organic phases were washed with saturated aqueous sodium carbonate solution and then with saturated aqueous sodium chloride solution, dried over sodium sulphate, filtered and concentrated on a rotary evaporator. This gave 2.5 g (73% of theory) of benzyl [4-(dimethylamino)-2,2-dimethylcyclohexyl]carbamate, which was used further without purification.

A solution of benzyl [4-(dimethylamino)-2,2-dimethylcyclohexyl]carbamate (3.0 g, 9.87 mmol) in ethanol (30 ml) was admixed with palladium/charcoal (10%, 1.5 g) and hydrogenated under 60 psi over the course of 18 h. Subsequently, the reaction mixture was filtered through Celite and the solvent was removed on a rotary evaporator. The 1.2 g (75% of theory) of N⁴,N⁴,2,2-tetramethylcyclohexane-1,4-diamine obtained were dissolved in diethyl ether (5 ml) and admixed with a 2M hydrochloride solution in diethyl ether (5 ml). The solvent was removed on a rotary evaporator. This gave 1.1 g (75% of theory) of the title compound (trans/cis mixture, about 3:1), which was used further without purification.

¹H NMR (400 MHz, DMSO-d₆): δ ppm 0.91-1.11 (m, 6H), 1.43-1.68 (m, 2H), 1.78-2.10 (m, 4H), 2.61-2.74 (m, 6H), 2.83-3.02 (m, 1H), 3.34-3.47 (m, 1H), 7.99-8.57 (m, 3H), 10.40-10.89 (m, 1H).

### Example 244A

### tert-Butyl [(trans-4-{[(2S)-3-(4′-{[4-(dimethylamino)-2,2-dimethylcyclohexyl]carbamoyl}-2′-methylbiphenyl-4-yl)-1-oxo-1-{[4-(1H-tetrazol-5-yl)phenyl]amino}propan-2-yl]carbamoyl}-cyclohexyl)methyl]carbamate

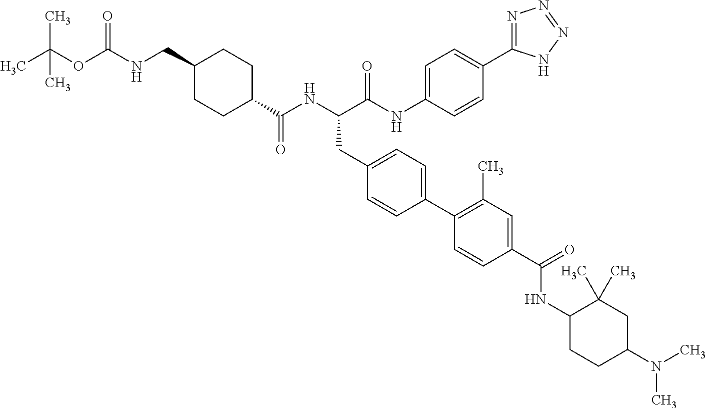 [SMILES: Cc1cc(C(=O)NC2CCC(N(C)C)CC2(C)C)ccc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 4′-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]-2-methylbiphenyl-4-carboxylic acid (90 mg, 0.13 mmol) and N⁴,N⁴,2,2-tetramethylcyclohexane-1,4-diamine dihydrochloride (64.2 mg, 0.26 mmol) in DMF (1 ml) was admixed with N,N-diisopropylamine (0.12 ml, 0.66 mmol), and HATU (75.3 mg, 0.20 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 45.3 mg (41% of theory) of the title compound.

LC-MS (Method 1): Rₜ=0.83 min; MS (ESIneg): m/z=832 [M−H]⁻.

### Example 245A

### tert-Butyl [(trans-4-{[(2S)-1-(1H-indazol-6-ylamino)-3-(2′-methyl-4′-{[(3R)-2-oxopyrrolidin-3-yl]carbamoyl}biphenyl-4-yl)-1-oxopropan-2-yl]carbamoyl}cyclohexyl)methyl]carbamate

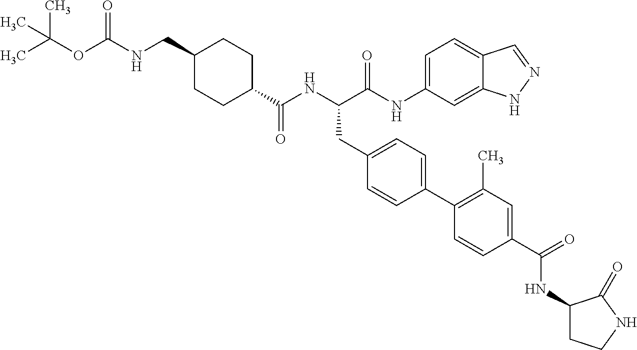 [SMILES: Cc1cc(C(=O)N[C@@H]2CCNC2=O)ccc1-c1ccc(C[C@H](NC(=O)[C@H]2CC[C@H](CNC(=O)OC(C)(C)C)CC2)C(=O)Nc2ccc3cn[nH]c3c2)cc1]

A solution of 4′-[(2S)-2-{[(trans-4-{[(tert-butoxycarbonyl)amino]methyl}cyclohexyl)-carbonyl]amino}-3-(1H-indazol-6-ylamino)-3-oxopropyl]-2-methylbiphenyl-4-carboxylic acid (100 mg, 0.15 mmol) and (3R)-3-aminopyrrolidin-2-one (18.3 mg, 0.18 mmol) in THF (5 ml) was admixed with N,N-diisopropylamine (0.03 ml, 0.18 mmol), and HATU (70 mg, 0.18 mmol) was added thereto. The reaction mixture was stirred at RT overnight (about 16 h). The solvent was removed on a rotary evaporator and the residue was dissolved in water/acetonitrile. The solution was filtered through a Millipore filter, then purified by preparative HPLC (eluent: gradient of acetonitrile/water with 0.1% trifluoroacetic acid). This gave 93 mg (83% of theory) of the title compound.

LC-MS (Method 1): Rₜ=0.91 min; MS (ESIneg): m/z=734 [M−H]⁻.

### Example 246A

### N-Benzyl-N-methylcyclohexane-1,4-diamine dihydrochloride

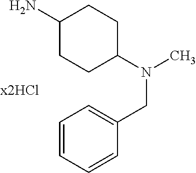 [SMILES: CN(Cc1ccccc1)C1CCC(N)CC1]

To a solution of tert-butyl (4-oxocyclohexyl)carbamate (750 mg, 3.52 mmol) and N-methyl-1-phenylmethanamine (426 mg, 3.52 mmol) in 1,2-dichloroethane (12.5 ml) were added sodium triacetoxyborhydride (1.04 g, 4.92 mmol) and acetic acid (0.2 ml, 3.52 mmol), and the mixture was stirred at RT overnight. The reaction mixture was admixed with a little 1N aqueous sodium hydroxide solution and added to water. The aqueous phase was extracted (three times) with ethyl acetate and the combined organic phases were washed with saturated aqueous sodium chloride solution, then dried over sodium sulphate, filtered and concentrated on a rotary evaporator. This gave 1.087 g (95% of theory) of tert-butyl {4-[benzyl(methyl)amino]cyclohexyl}carbamate, which was used further without purification. A solution of tert-butyl {4-[benzyl(methyl)amino]cyclohexyl}carbamate (1.044 g, 3.28 mmol) in dioxane (30 ml) was admixed with 4M hydrogen chloride in 1,4-dioxane (8.2 ml, 32.8 mmol) and stirred at RT overnight. The precipitated solid was filtered off and washed with dioxane. The solid was dried under high vacuum. This gave 953 mg (99% of theory) of the title compound.

LC-MS (Method 12): Rₜ=1.94 min; MS (ESIpos): m/z=218 [M+H-2HCl]⁺.

### Example 247A

### tert-Butyl [(trans-4-{[(2S)-3-[4′-({4-[benzyl(methyl)amino]cyclohexyl}carbamoyl)-2′-methyl-biphenyl-4-yl]-1-oxo-1-{[4-(1H-tetrazol-5-yl)phenyl]amino}propan-2-yl]carbamoyl}cyclohexyl)-methyl]carbamate

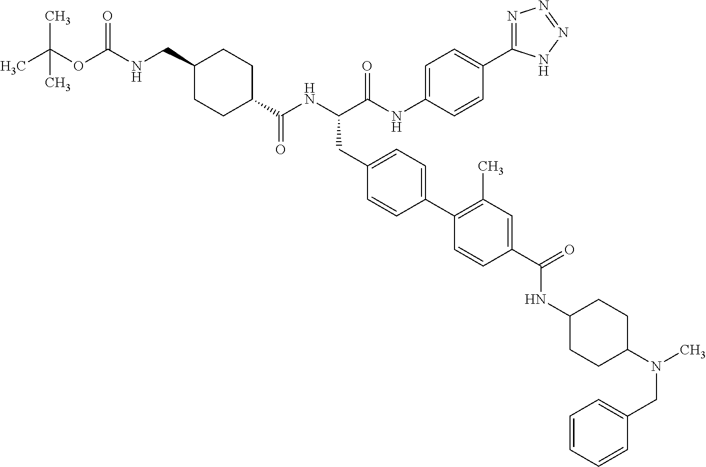 [SMILES: Cc1cc(C(=O)NC2CCC(N(C)Cc3ccccc3)CC2)ccc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 4′-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]-2-methylbiphenyl-4-carboxylic acid (200 mg, 0.29 mmol) and N-benzyl-N-methylcyclohexane-1,4-diamine dihydrochloride (170 mg, 0.59 mmol) in DMF (2 ml) was admixed with N,N-diisopropylamine (0.26 ml, 1.47 mmol), and HATU (167 mg, 0.44 mmol) was added thereto. The reaction mixture was stirred at RT overnight (about 16 h). The solvent was removed on a rotary evaporator and the residue was dissolved in water/acetonitrile. The solution was filtered through a Millipore filter, then purified by preparative HPLC (eluent: gradient of acetonitrile/water with 0.1% trifluoroacetic acid). This gave 131.4 mg (50% of theory) of the title compound.

LC-MS (Method 18): Rₜ=1.06 min; MS (ESIpos): m/z=882 [M+H]⁺.

### Example 248A

### tert-Butyl [(trans-4-{[(2S)-3-(2′-methyl-4′-{[4-(methylamino)cyclohexyl]carbamoyl}biphenyl-4-yl)-1-oxo-1-{[4-(1H-tetrazol-5-yl)phenyl]amino}propan-2-yl]carbamoyl}cyclohexyl)methyl]-carbamate

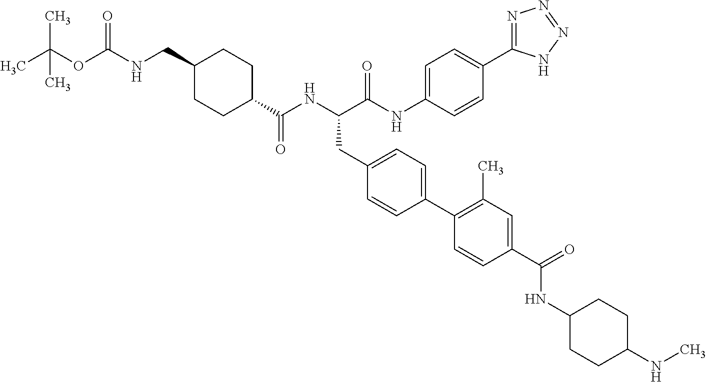 [SMILES: CNC1CCC(NC(=O)c2ccc(-c3ccc(C[C@H](NC(=O)[C@H]4CC[C@H](CNC(=O)OC(C)(C)C)CC4)C(=O)Nc4ccc(-c5nnn[nH]5)cc4)cc3)c(C)c2)CC1]

A suspension of tert-butyl [(trans-4-{[(2S)-3-[4′-({4-[benzyl(methyl)amino]cyclohexyl}-carbamoyl)-2′-methylbiphenyl-4-yl]-1-oxo-1-{[4-(1H-tetrazol-5-yl)phenyl]amino}propan-2-yl]carbamoyl}cyclohexyl)methyl]carbamate (82.5 mg, 0.094 mmol) and palladium/charcoal (100/o %, 20 mg, 0.019 mmol) in ethyl acetate (2 ml) and methanol (2 ml) was hydrogenated at RT under standard pressure over the course of 12 h. The solution was diluted with a little methanol, filtered through a Millipore filter and purified by preparative HPLC (eluent: gradient of acetonitrile/water with 0.1% trifluoroacetic acid). This gave 64.9 mg (86% of theory) of the title compound.

LC-MS (Method 1): Rₜ=0.81 min; MS (ESIneg): m/z=790 [M−H]⁻.

### Example 249A

### 4-{5-[4-({4-Bromo-N-[(trans-4-{[(tert-butoxycarbonyl)amino]methyl}cyclohexyl)carbonyl]-L-phenylalanyl}amino)phenyl]-4H-1,2,4-triazol-3-yl}-2,2,3,3,4,4-hexafluorobutanoic acid

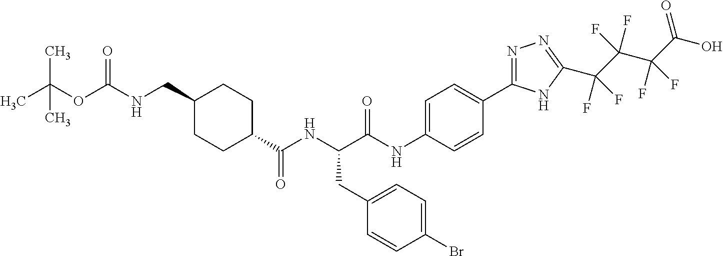 [SMILES: CC(C)(C)OC(=O)NC[C@H]1CC[C@H](C(=O)N[C@@H](Cc2ccc(Br)cc2)C(=O)Nc2ccc(-c3nnc(C(F)(F)C(F)(F)C(F)(F)C(=O)O)[nH]3)cc2)CC1]

A solution of 4-bromo-N-[(trans-4-{[(tert-butoxycarbonyl)amino]methyl}cyclohexyl)carbonyl]-L-phenylalanine (1.02 g, 2.12 mmol) and 4-[5-(4-aminophenyl)-4H-1,2,4-triazol-3-yl]-2,2,3,3,4,4-hexafluorobutanoic acid hydrochloride (1.81 g, 4.24 mmol) in DMF (15 ml) was admixed with N,N-diisopropylamine (1.84 ml, 10.59 mmol), and HATU (1.21 g, 3.18 mmol) was added thereto. The reaction mixture was stirred at RT overnight (about 16 h). The solvent was removed on a rotary evaporator and the residue was dissolved in DMSO. The solution was diluted with a little acetonitrile, filtered through a Millipore filter and purified by preparative HPLC (eluent: gradient of acetonitrile/water with 0.1% trifluoroacetic acid). This gave 556 mg (30% of theory) of the title compound.

¹H NMR (400 MHz, DMSO-d₆): δ=ppm 0.82 (m, 2H), 1.08-1.29 (m, 3H), 1.37 (s, 9H), 1.51-1.59 (m, 1H), 1.61-1.75 (m, 3H), 2.02-2.17 (m, 1H), 2.75 (m, 2H), 2.80-2.89 (m, 1H), 3.02 (dd, 1H), 4.65 (d, 1H), 6.73-6.83 (m, 1H), 7.26 (d, 2H), 7.48 (d, 2H), 7.77 (d, 2H), 7.97 (d, 2H), 8.14 (d, 1H), 10.41 (s, 1H), 15.15 (br. s, 1H).

LC-MS (Method 1): Rₜ=1.04 min; MS (ESIpos): m/z=821 [M+H]⁺.

### Example 250A

### N-[4-(Dimethylamino)cyclohexyl]-3-methyl-4-(4,4,5,5-tetramethyl-1,3,2-dioxaborolan-2-yl)benzamide

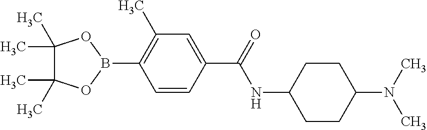

A solution of 3-methyl-4-(4,4,5,5-tetramethyl-1,3,2-dioxaborolan-2-yl)benzoic acid (500 mg, 1.91 mmol) and N,N-dimethylcyclohexane-1,4-diamine (380 mg, 2.67 mmol) in DMF (17 ml) was admixed with N,N-diisopropylamine (1.0 ml, 5.72 mmol), and HATU (1.45 g, 3.82 mmol) was added thereto. The reaction mixture was stirred at RT overnight. The solvent was removed on a rotary evaporator and the residue was dissolved in DMSO. The solution was diluted with a little acetonitrile, filtered through a Millipore filter and purified by preparative HPLC (eluent: gradient of acetonitrile/water with 0.1% trifluoroacetic acid). This gave 380 mg (52% of theory) of the title compound (about 30% as the boronic acid).

LC-MS (Method 18): Rₜ=0.97 min; MS (ESIpos): m/z=387 [M+H]⁺.

### Example 251A

### 4-[5-(4-{[(2S)-2-{[(trans-4-{[(tert-Butoxycarbonyl)amino]methyl}cyclohexyl)carbonyl]amino}-3-(4′-{[4-(dimethylamino)cyclohexyl]carbamoyl}-2′-methylbiphenyl-4-yl)propanoyl]amino}phenyl)-4H-1,2,4-triazol-3-yl]-2,2,3,3,4,4-hexafluorobutanoic acid

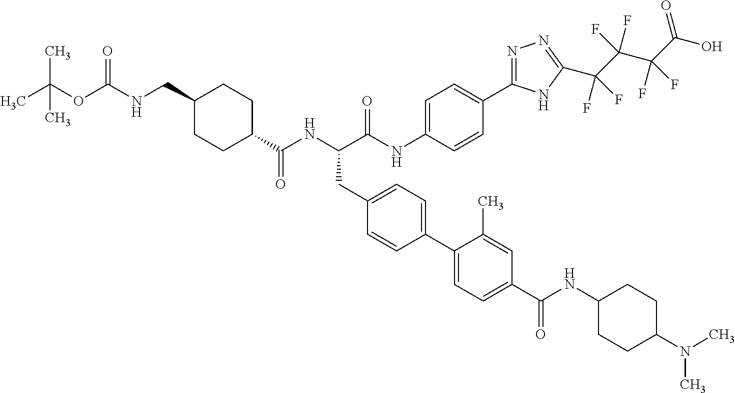 [SMILES: Cc1cc(C(=O)NC2CCC(N(C)C)CC2)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(F)(F)C(=O)O)[nH]3)cc2)cc1]

To a solution of 4-{5-[4-({4-bromo-N-[(trans-4-{[(tert-butoxycarbonyl)amino]methyl}-cyclohexyl)carbonyl]-L-phenylalanyl}amino)phenyl]-4H-1,2,4-triazol-3-yl}-2,2,3,3,4,4-hexafluorobutanoic acid (100 mg, 0.122 mmol) and N-[4-(dimethylamino)cyclohexyl]-3-methyl-4-(4,4,5,5-tetramethyl-1,3,2-dioxaborolan-2-yl)benzamide (66 mg, 0.171 mmol) in 1,2-dimethoxyethane (1 ml) were added ethanol (0.4 ml), 2N aqueous sodium carbonate solution (0.12 ml, 0.24 mmol) and [1,1-bis-(diphenylphosphino)ferrocene]dichloropalladium-dichloromethane complex (5 mg, 0.006 mmol). The mixture was then stirred at reflux (oil bath temperature 100° C.) for 8 h. The reaction mixture was concentrated on a rotary evaporator and the residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 12 mg (8% of theory) of the title compound.

LC-MS (Method 1): Rₜ=0.86 min; MS (ESIpos): m/z=999 [M+H]⁺.

### Example 252A

### tert-Butyl 4-[({4′-[(2S)-2-{[(trans-4-{[(tert-butoxycarbonyl)amino]methyl}cyclohexyl)carbonyl]-amino}-3-oxo-3-{[4-(1H-tetrazol-5-yl)phenyl]amino}propyl]-2-methylbiphenyl-4-yl}-carbonyl)amino]-3,3-dimethylpiperidine-1-carboxylate

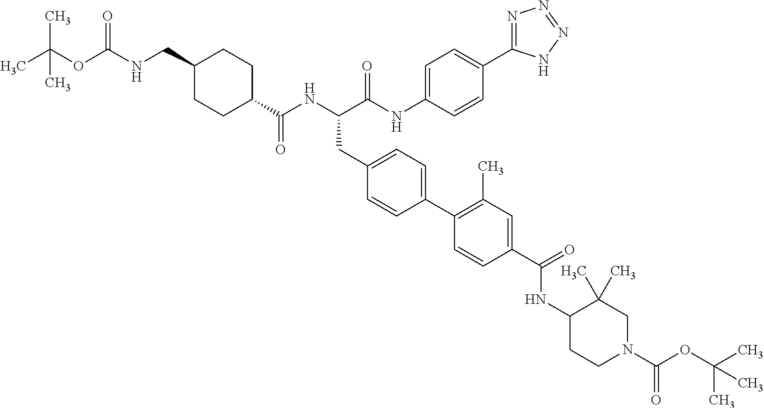 [SMILES: Cc1cc(C(=O)NC2CCN(C(=O)OC(C)(C)C)CC2(C)C)ccc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 4′-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]-2-methylbiphenyl-4-carboxylic acid (100 mg, 0.15 mmol) and tert-butyl 4-amino-3,3-dimethylpiperidine-1-carboxylate (67 mg, 0.29 mmol) in DMF (1.5 ml) was admixed with N,N-diisopropylamine (0.05 ml, 0.29 mmol), and HATU (67 mg, 0.18 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 80 mg (60% of theory) of the title compound.

LC-MS (Method 1): Rₜ=1.22 min; MS (ESIneg): m/z=890 [M−H]⁻.

### Example 253A

### tert-Butyl [(trans-4-{[(2S)-3-[4′-({4-[ethyl(methyl)amino]cyclohexyl}carbamoyl)-2′-methyl-biphenyl-4-yl]-1-oxo-1-{[4-(1H-tetrazol-5-yl)phenyl]amino}propan-2-yl]carbamoyl}-cyclohexyl)methyl]carbamate

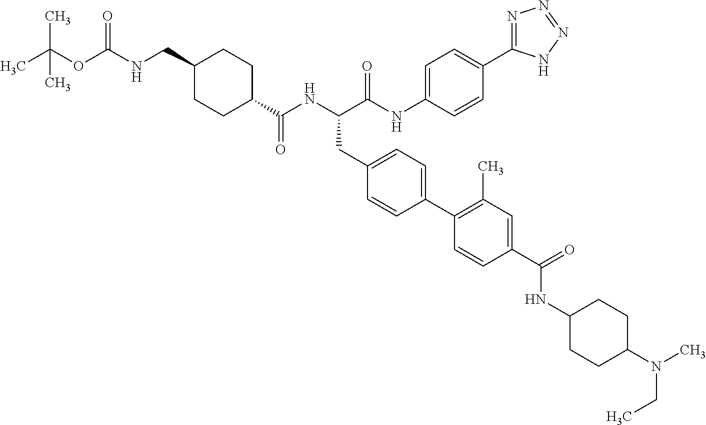 [SMILES: CCN(C)C1CCC(NC(=O)c2ccc(-c3ccc(C[C@H](NC(=O)[C@H]4CC[C@H](CNC(=O)OC(C)(C)C)CC4)C(=O)Nc4ccc(-c5nnn[nH]5)cc4)cc3)c(C)c2)CC1]

A solution of 4′-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]-2-methylbiphenyl-4-carboxylic acid (100 mg, 0.15 mmol) and N-ethyl-N-methylcyclohexane-1,4-diamine dihydrochloride (67 mg, 0.29 mmol) in DMF (1 ml) was admixed with N,N-diisopropylamine (0.13 ml, 0.733 mmol), and HATU (84 mg, 0.22 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 59.1 mg (47% of theory) of the title compound.

LC-MS (Method 1): Rₜ=0.82 min; MS (ESIneg): m/z=818 [M−H]⁻.

### Example 254A

### Methyl 5-[({4′-[(2S)-2-{[(trans-4-{[(tert-butoxycarbonyl)amino]methyl}cyclohexyl)carbonyl]-amino}-3-oxo-3-{[4-(1H-tetrazol-5-yl)phenyl]amino}propyl]-2-methylbiphenyl-4-yl}carbonyl)-amino]-6-oxopiperidine-2-carboxylate

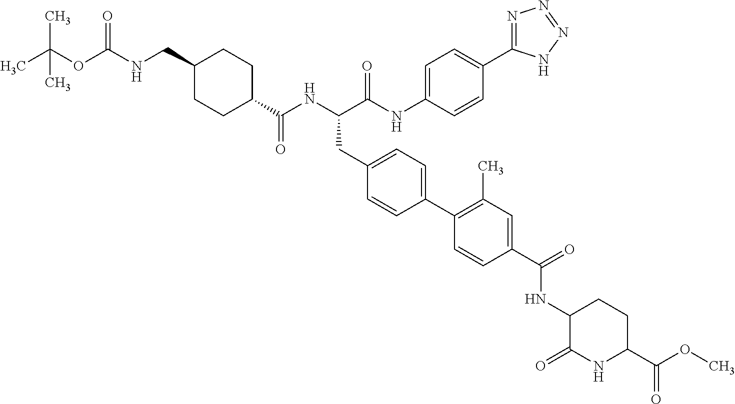 [SMILES: COC(=O)C1CCC(NC(=O)c2ccc(-c3ccc(C[C@H](NC(=O)[C@H]4CC[C@H](CNC(=O)OC(C)(C)C)CC4)C(=O)Nc4ccc(-c5nnn[nH]5)cc4)cc3)c(C)c2)C(=O)N1]

A solution of 4′-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]-2-methylbiphenyl-4-carboxylic acid (100 mg, 0.15 mmol) and methyl 5-amino-6-oxopiperidine-2-carboxylate (51 mg, 0.29 mmol) in DMF (1.5 ml) was admixed with N,N-diisopropylamine (0.05 ml, 0.29 mmol), and HATU (67 mg, 0.18 mmol) was added thereto. The reaction mixture was stirred at RT overnight (about 16 h). Additional methyl 5-amino-6-oxopiperidine-2-carboxylate (25 mg, 0.15 mmol) and HATU (28 mg, 0.073 mmol) were added and the reaction solution was stirred at RT for a further 24 h. The residue was diluted with water/acetonitrile and filtered through a Millipore filter, then purified by preparative HPLC (eluent: gradient of acetonitrile/water with 0.1% trifluoroacetic acid). This gave 40 mg (31% of theory) of the title compound.

LC-MS (Method 1): Rₜ=1.22 min; MS (ESIneg): m/z=890 [M−H]⁻.

### Example 255A

### tert-Butyl 4-(2-[(4-nitrophenyl)carbonoimidoyl]hydrazino)-4-oxobutanoate

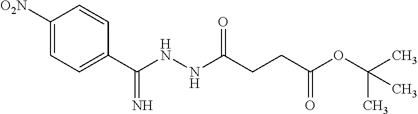 [SMILES: CC(C)(C)OC(=O)CCC(=O)NNC(=N)c1ccc([N+](=O)[O-])cc1]

A solution of 4-nitrobenzenecarboximidohydrazide hydrochloride (1.7 g, 9.57 mmol) and 4-tert-butoxy-4-oxobutanoic acid (1.67 g, 9.57 mmol) in THF (35 ml) was admixed with HATU (67 mg, 0.18 mmol). The reaction mixture was stirred at RT overnight (about 16 h). The solvent was removed on a rotary evaporator and the residue was dissolved in methanol, filtered through a Millipore filter and purified by preparative HPLC (eluent: gradient of acetonitrile/water with 0.1% trifluoroacetic acid). This gave 1.47 g (35% of theory) of the title compound.

LC-MS (Method 1): Rₜ=0.62 min; MS (ESIneg): m/z=335 [M−H]⁻.

### Example 256A

### tert-Butyl 3-[5-(4-nitrophenyl)-4H-1,2,4-triazol-3-yl]propanoate

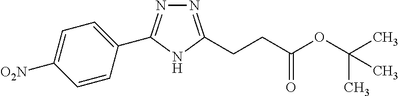 [SMILES: CC(C)(C)OC(=O)CCc1nnc(-c2ccc([N+](=O)[O-])cc2)[nH]1]

A solution of tert-butyl 4-{2-[(4-nitrophenyl)carbonoimidoyl]hydrazino}-4-oxobutanoate (1.46 g, 4.34 mmol) in 1-methylpyrrolidine (15 ml) was stirred at 120° C. for 3 days. The solvent was removed on a rotary evaporator. The residue was recrystallized from methanol and the solid was filtered off with suction, washed with a little methanol and dried under high vacuum. This gave 657 mg (44% of theory) of the title compound.

LC-MS (Method 1): Rₜ=0.95 min; MS (ESIneg): m/z=317 [M−H]⁻.

### Example 257A

### tert-Butyl 3-[5-(4-aminophenyl)-4H-1,2,4-triazol-3-yl]propanoate

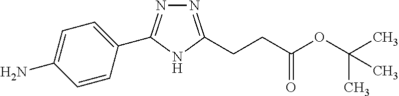 [SMILES: CC(C)(C)OC(=O)CCc1nnc(-c2ccc(N)cc2)[nH]1]

A solution of tert-butyl 3-[5-(4-nitrophenyl)-4H-1,2,4-triazol-3-yl]propanoate (622 mg, 1.95 mmol) and tin(II) chloride hydrate (1.76 g, 7.82 mmol) in ethanol (20 ml) was stirred at 70° C. for 1 h. The solution was cooled to RT and poured onto ice-water, then adjusted to pH 8 with sodium carbonate. The mixture was diluted with water and extracted with ethyl acetate. The combined organic phases were washed with saturated aqueous sodium chloride solution, dried over sodium sulphate, filtered and concentrated on a rotary evaporator. This gave 537.7 mg (93% of theory) of the title compound, which was used further without purification.

LC-MS (Method 1): Rₜ=0.65 min; MS (ESIneg): m/z=287 [M−H]⁻.

### Example 258A

### tert-Butyl 4-[({4′-[(2S)-2-{[(trans-4-{[(tert-butoxycarbonyl)amino]methyl}cyclohexyl)-carbonyl]amino}-3-({4-[5-(3-tert-butoxy-3-oxopropyl)-4H-1,2,4-triazol-3-yl]phenyl}amino)-3-oxopropyl]-2-methylbiphenyl-4-yl}carbonyl)amino]piperidine-1-carboxylate

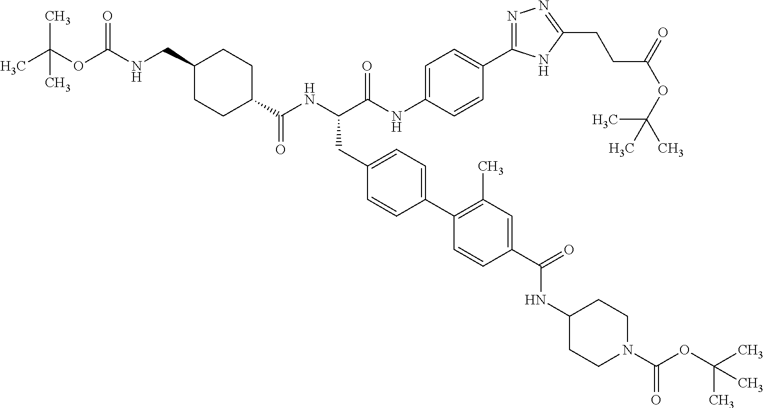 [SMILES: Cc1cc(C(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc(-c3nnc(CCC(=O)OC(C)(C)C)[nH]3)cc2)cc1]

A solution of (2S)-2-{[(trans-4-{[(tert-butoxycarbonyl)amino]methyl}cyclohexyl)-carbonyl]amino}-3-(4′-{[1-(tert-butoxycarbonyl)piperidin-4-yl]carbamoyl}-2′-methylbiphenyl-4-yl)propanoic acid (100 mg, 0.14 mmol) and tert-butyl 3-[5-(4-aminophenyl)-4H-1,2,4-triazol-3-yl]propanoate (80 mg, 0.28 mmol) in DMF (2 ml) was admixed with N,N-diisopropylamine (0.07 ml, 0.42 mmol), and HATU (79 mg, 0.21 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 73 mg (53% of theory) of the title compound.

LC-MS (Method 1): Rₜ=1.21 min; MS (ESIneg): m/z=989 [M−H]⁻.

### Example 259A

### 3-[5-(4-{[(2S)-2-{[(trans-4-{[(tert-Butoxycarbonyl)amino]methyl}cyclohexyl)carbonyl]amino}-3-(4′-{[(3S)-1-(tert-butoxycarbonyl)pyrrolidin-3-yl]carbamoyl}-2′-methylbiphenyl-4-yl)propanoyl]amino}phenyl)-4H-1,2,4-triazol-3-yl]-2,2,3,3-tetrafluoropropanoic acid

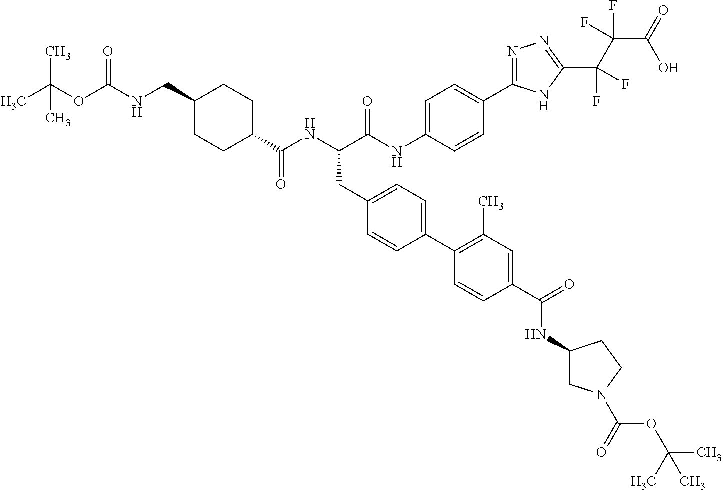 [SMILES: Cc1cc(C(=O)N[C@H]2CCN(C(=O)OC(C)(C)C)C2)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

A solution of 4′-[(2S)-2-{[(trans-4-{[(tert-butoxycarbonyl)amino]methyl}cyclohexyl)-carbonyl]amino}-3-({4-[5-(2-carboxy-1,1,2,2-tetrafluoroethyl)-4H-1,2,4-triazol-3-yl]phenyl}-amino)-3-oxopropyl]-2-methylbiphenyl-4-carboxylic acid (80 mg, 0.097 mmol) and tert-butyl (3S)-3-aminopyrrolidine-1-carboxylate (36 mg, 0.19 mmol) in DMF (1 ml) was admixed with N,N-diisopropylamine (0.05 ml, 0.29 mmol), and HATU (55 mg, 0.15 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 61.9 mg (60% of theory) of the title compound.

LC-MS (Method 1): Rₜ=1.09 min; MS (ESIneg): m/z=991 [M−H]⁻.

### Example 260A

### 3-[5-(4-{[(2S)-2-{[(trans-4-{[(tert-Butoxycarbonyl)amino]methyl}cyclohexyl)carbonyl]amino}-3-(4′-{[3-(tert-butoxycarbonyl)-3-azabicyclo[3.1.0]hex-6-yl]carbamoyl}-2′-methylbiphenyl-4-yl)propanoyl]amino}phenyl)-4H-1,2,4-triazol-3-yl]-2,2,3,3-tetrafluoropropanoic acid

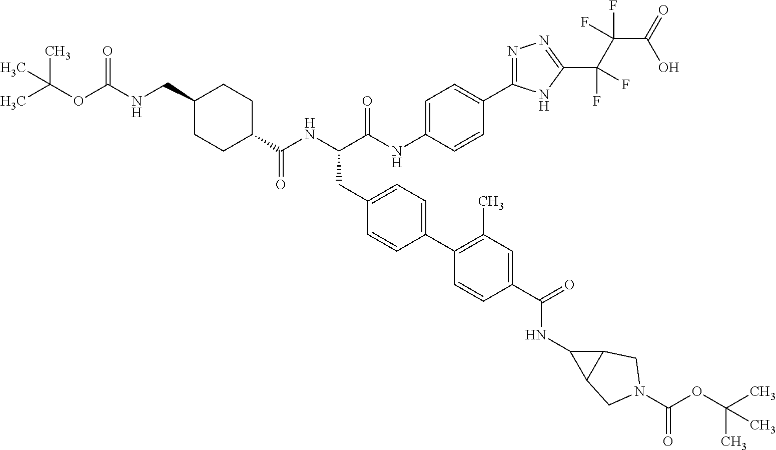 [SMILES: Cc1cc(C(=O)NC2C3CN(C(=O)OC(C)(C)C)CC32)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

A solution of 4′-[(2S)-2-{[(trans-4-{[(tert-butoxycarbonyl)amino]methyl}cyclohexyl)-carbonyl]amino}-3-({4-[5-(2-carboxy-1,1,2,2-tetrafluoroethyl)-4H-1,2,4-triazol-3-yl]phenyl}-amino)-3-oxopropyl]-2-methylbiphenyl-4-carboxylic acid (80 mg, 0.097 mmol) and tert-butyl 6-amino-3-azabicyclo[3.1.0]hexane-3-carboxylate (38 mg, 0.19 mmol) in DMF (1 ml) was admixed with N,N-diisopropylamine (0.05 ml, 0.29 mmol), and HATU (55 mg, 0.15 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 61.9 mg (60% of theory) of the title compound.

LC-MS (Method 1): Rₜ=1.10 min; MS (ESIneg): m/z=1003 [M−H]⁻.

### Example 261A

### 3-[5-(4-{[(2S)-2-{[(trans-4-{[(tert-Butoxycarbonyl)amino]methyl}cyclohexyl)carbonyl]amino}-3-{2′-methyl-4′-[(2-oxopiperidin-3-yl)carbamoyl]biphenyl-4-yl}propanoyl]amino}phenyl)-4H-1,2,4-triazol-3-yl]-2,2,3,3-tetrafluoropropanoic acid

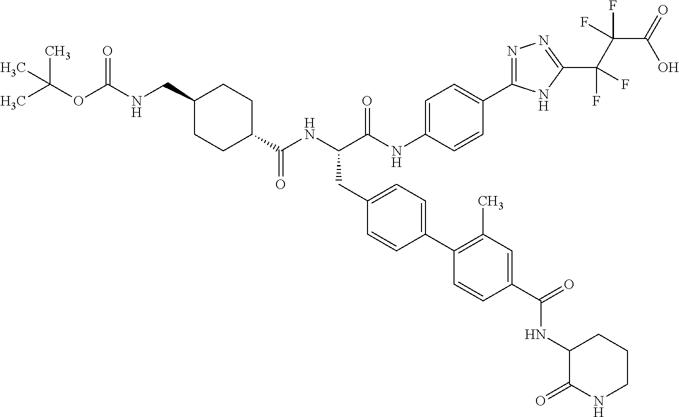 [SMILES: Cc1cc(C(=O)NC2CCCNC2=O)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

A solution of 4′-[(2S)-2-{[(trans-4-{[(tert-butoxycarbonyl)amino]methyl}cyclohexyl)-carbonyl]amino}-3-({4-[5-(2-carboxy-1,1,2,2-tetrafluoroethyl)-4H-1,2,4-triazol-3-yl]phenyl}-amino)-3-oxopropyl]-2-methylbiphenyl-4-carboxylic acid (80 mg, 0.097 mmol) and 3-aminopiperidin-2-one (22 mg, 0.19 mmol) in DMF (1 ml) was admixed with N,N-diisopropylamine (0.05 ml, 0.29 mmol), and HATU (55 mg, 0.15 mmol) was added thereto. The reaction mixture was stirred at RT overnight (about 16 h). The residue was diluted with water/acetonitrile and filtered through a Millipore filter, then purified by preparative HPLC (eluent: gradient of acetonitrile/water with 0.1% trifluoroacetic acid). This gave 61.9 mg (60% of theory) of the title compound.

LC-MS (Method 1): Rₜ=0.89 min; MS (ESIneg): m/z=919 [M−H]⁻.

### Example 262A

### tert-Butyl {[trans-4-({(2S)-1-[(7-chloro-2-oxo-2,3-dihydro-1,3-benzoxazol-5-yl)amino]-3-[4′-(isopropylcarbamoyl)-2′-methylbiphenyl-4-yl]-1-oxopropan-2-yl}carbamoyl)cyclohexyl]methyl}-carbamate

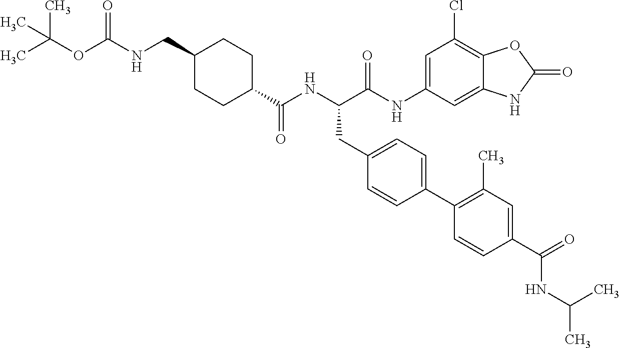 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2cc(Cl)c3oc(=O)[nH]c3c2)cc1]

A suspension of (2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoic acid (100 mg, 0.172 mmol) in ethyl acetate (2.5 ml) was admixed with 5-amino-7-chloro-1,3-benzoxazol-2(3H)-one (35 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 28.8 mg (22% of theory) of the title compound.

LC-MS (Method 1): Rₜ=1.09 min; MS (ESIneg): m/z=744 [M−H]⁻.

### Example 263A

### tert-Butyl 3-{5-[4-({(2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoyl}amino)phenyl]-4H-1,2,4-triazol-3-yl}propanoate

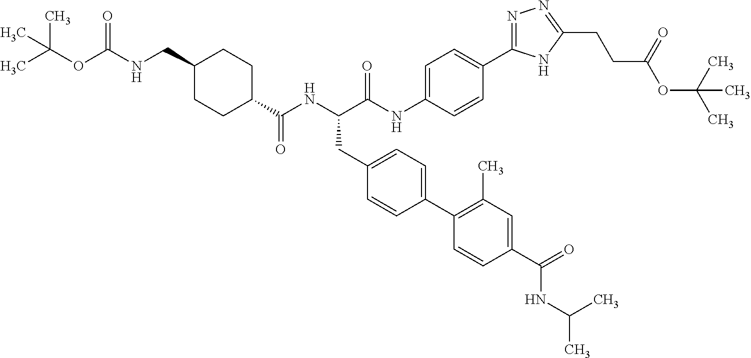 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2ccc(-c3nnc(CCC(=O)OC(C)(C)C)[nH]3)cc2)cc1]

A suspension of (2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoic acid (100 mg, 0.172 mmol) in ethyl acetate (2.5 ml) was admixed with tert-butyl 3-[5-(4-aminophenyl)-4H-1,2,4-triazol-3-yl]propanoate (54 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 65.2 mg (44% of theory) of the title compound.

LC-MS (Method 1): Rₜ=1.11 min; MS (ESIneg): m/z=848 [M−H]⁻.

### Example 264A

### tert-Butyl {[trans-4-({(2S)-1-[(7-chloro-2-oxo-2,3-dihydro-1H-benzimidazol-5-yl)amino]-3-[4′-(isopropylcarbamoyl)-2′-methylbiphenyl-4-yl]-1-oxopropan-2-yl}carbamoyl)cyclohexyl]methyl}-carbamate

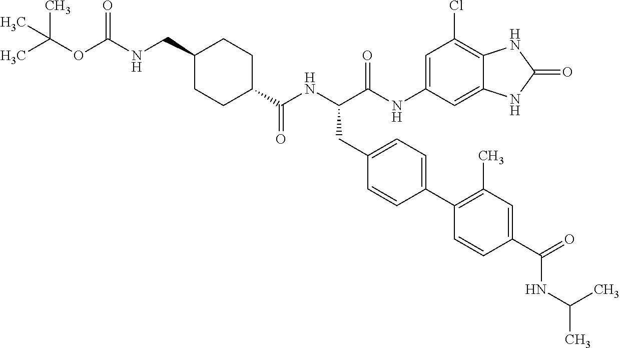 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2cc(Cl)c3[nH]c(=O)[nH]c3c2)cc1]

A suspension of (2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoic acid (100 mg, 0.172 mmol) in ethyl acetate (2.5 ml) was admixed with 6-amino-4-chloro-1,3-dihydro-2H-benzimidazol-2-one (35 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 80 mg (60% of theory) of the title compound.

LC-MS (Method 1): Rₜ=1.01 min; MS (ESIneg): m/z=743 [M−H]⁻.

### Example 265A

### tert-Butyl {[trans-4-({(2S)-1-[(4-chloro-1H-indazol-6-yl)amino]-3-[4′-(isopropylcarbamoyl)-2-methylbiphenyl-4-yl]-1-oxopropan-2-yl}carbamoyl)cyclohexyl]methyl}carbamate

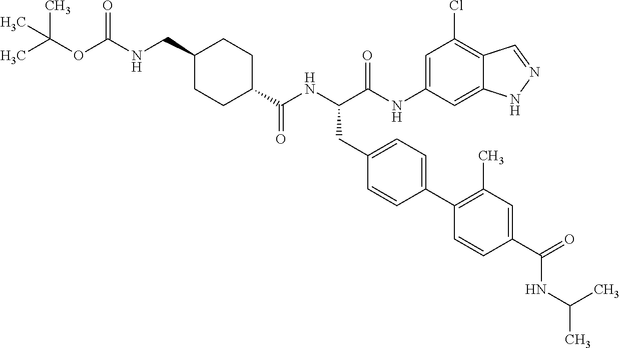 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2cc(Cl)c3cn[nH]c3c2)cc1]

A suspension of (2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoic acid (100 mg, 0.172 mmol) in ethyl acetate (2.5 ml) was admixed with 4-chloro-1H-indazol-6-amine (35 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 22 mg (14% of theory) of the title compound.

LC-MS (Method 1): Rₜ=1.15 min; MS (ESIneg): m/z=727 [M−H]⁻.

### Example 266A

### Ethyl 6-({(2S)-2-{[(trans-4-{[(tert-butoxycarbonyl)amino]methyl}cyclohexyl)carbonyl]amino}-3-[4′-(cyclobutylcarbamoyl)-2′-methylbiphenyl-4-yl]propanoyl}amino)-1H-indole-2-carboxylate

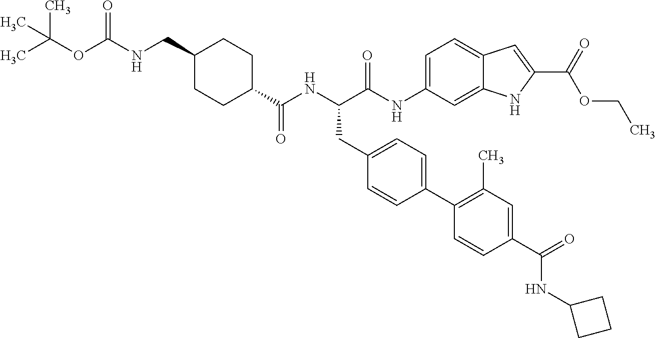 [SMILES: CCOC(=O)c1cc2ccc(NC(=O)[C@H](Cc3ccc(-c4ccc(C(=O)NC5CCC5)cc4C)cc3)NC(=O)[C@H]3CC[C@H](CNC(=O)OC(C)(C)C)CC3)cc2[nH]1]

A suspension of (2S)-2-{[(trans-4-{[(tert-butoxycarbonyl)amino]methyl}cyclohexyl)-carbonyl]amino}-3-[4′-(cyclobutylcarbamoyl)-2′-methylbiphenyl-4-yl]propanoic acid (120 mg, 0.20 mmol) in ethyl acetate (3 ml) was admixed with ethyl 6-amino-1H-indole-2-carboxylate (46 mg, 0.22 mmol) and N,N-diisopropylethylamine (0.11 ml, 0.61 mmol). The suspension was admixed with a 2,4,6-tripropyl-1,3,5,2,4,6-trioxatriphosphinane 2,4,6-trioxide solution (50% in DMF, 0.36 ml, 0.61 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 68 mg (41% of theory) of the title compound.

LC-MS (Method 1): Rₜ=1.24 min; MS (ESIneg): m/z=776 [M−H]⁻.

### Example 267A

### 6-({(2S)-2-{[(trans-4-{[(tert-Butoxycarbonyl)amino]methyl}cyclohexyl)carbonyl]amino}-3-[4′-(cyclobutylcarbamoyl)-2′-methylbiphenyl-4-yl]propanoyl}amino)-1H-indole-2-carboxylic acid

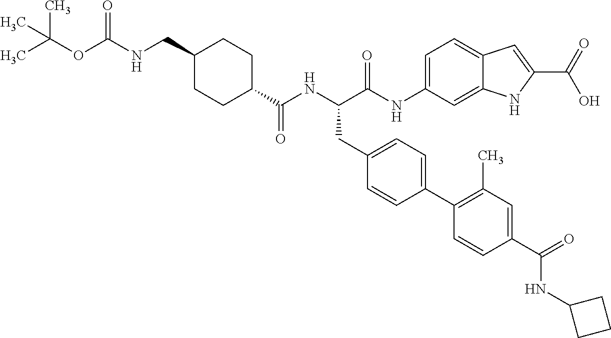 [SMILES: Cc1cc(C(=O)NC2CCC2)ccc1-c1ccc(C[C@H](NC(=O)[C@H]2CC[C@H](CNC(=O)OC(C)(C)C)CC2)C(=O)Nc2ccc3cc(C(=O)O)[nH]c3c2)cc1]

Ethyl 6-({(2S)-2-{[(trans-4-{[(tert-butoxycarbonyl)amino]methyl}cyclohexyl)carbonyl]amino}-3-[4′-(cyclobutylcarbamoyl)-2′-methylbiphenyl-4-yl]propanoyl}amino)-1H-indole-2-carboxylate (50 mg, 0.064 mmol) was dissolved in tetrahydrofuran/water 3/1 (2 ml), admixed with lithium hydroxide monohydrate (27 mg, 0.64 mmol) and stirred at RT overnight, then stirred at 60° C. for a further 10 h. The reaction mixture was concentrated on a rotary evaporator. The residue was dissolved in DMSO (1 ml) and acetonitrile (3 ml), filtered through a Millipore filter and purified by preparative HPLC (eluent: gradient of acetonitrile/water with 0.1% trifluoroacetic acid). This gave 27 mg (56% of theory) of the title compound.

LC-MS (Method 1): Rₜ=1.09 min; MS (ESIneg): m/z=748 [M−H]⁻.

### Example 268A

### Methyl 6-({(2S)-2-{[(trans-4-{[(tert-butoxycarbonyl)amino]methyl}cyclohexyl)carbonyl]amino}-3-[4′-(isopropylcarbamoyl)-2′-methylbiphenyl-4-yl]propanoyl}amino)-1H-indazole-4-carboxylate

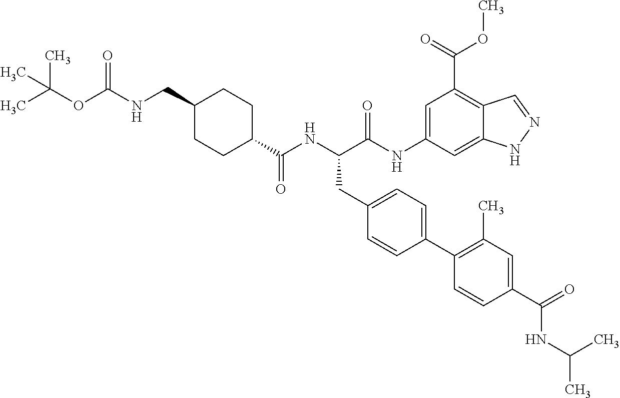 [SMILES: COC(=O)c1cc(NC(=O)[C@H](Cc2ccc(-c3ccc(C(=O)NC(C)C)cc3C)cc2)NC(=O)[C@H]2CC[C@H](CNC(=O)OC(C)(C)C)CC2)cc2[nH]ncc12]

A suspension of (2S)-2-{[(trans-4-{[(tert-butoxycarbonyl)amino]methyl}cyclohexyl)-carbonyl]amino}-3-[4′-(cyclobutylcarbamoyl)-2′-methylbiphenyl-4-yl]propanoic acid (200 mg, 0.35 mmol) in ethyl acetate (5 ml) was admixed with methyl 6-amino-1H-indazole-4-carboxylate (73 mg, 0.38 mmol) and N,N-diisopropylethylamine (0.18 ml, 0.60 mmol). The suspension was admixed with a 2,4,6-tripropyl-1,3,5,2,4,6-trioxatriphosphinane 2,4,6-trioxide solution (50% in DMF, 0.60 ml, 1.04 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 94 mg (35% of theory) of the title compound.

LC-MS (Method 1): Rₜ=1.07 min; MS (ESIneg): m/z=751 [M−H]⁻.

### Example 269A

### 6-({(2S)-2-{[(trans-4-{[(tert-Butoxycarbonyl)amino]methyl}cyclohexyl)carbonyl]amino}-3-[4′-(isopropylcarbamoyl)-2′-methylbiphenyl-4-yl]propanoyl}amino)-1H-indazole-4-carboxylic acid

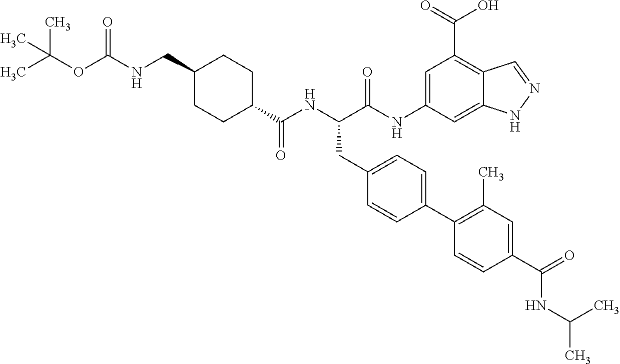 [SMILES: Cc1cc(C(=O)NC(C)C)ccc1-c1ccc(C[C@H](NC(=O)[C@H]2CC[C@H](CNC(=O)OC(C)(C)C)CC2)C(=O)Nc2cc(C(=O)O)c3cn[nH]c3c2)cc1]

Methyl 6-({(2S)-2-{[(trans-4-{[(tert-butoxycarbonyl)amino]methyl}cyclohexyl)carbonyl]amino}-3-[4′-(isopropylcarbamoyl)-2′-methylbiphenyl-4-yl]propanoyl}amino)-1H-indazole-4-carboxylate (88 mg, 0.12 mmol) was dissolved in tetrahydrofuran/water 3/1 (4 ml), admixed with lithium hydroxide monohydrate (49 mg, 1.17 mmol) and stirred at RT for 3 days. The reaction mixture was diluted with water, acidified slightly with 1N hydrochloric acid and concentrated on a rotary evaporator. The residue was dissolved in DMSO (1 ml) and acetonitrile (3 ml), filtered through a Millipore filter and purified by preparative HPLC (eluent: gradient of acetonitrile/water with 0.1% trifluoroacetic acid). This gave 20 mg (23% of theory) of the title compound.

LC-MS (Method 1): Rₜ=0.98 min; MS (ESIneg): m/z=737 [M−H]⁻.

### Example 270A

### tert-Butyl {[trans-4-({(2S)-3-[4′-(cyclobutylcarbamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate

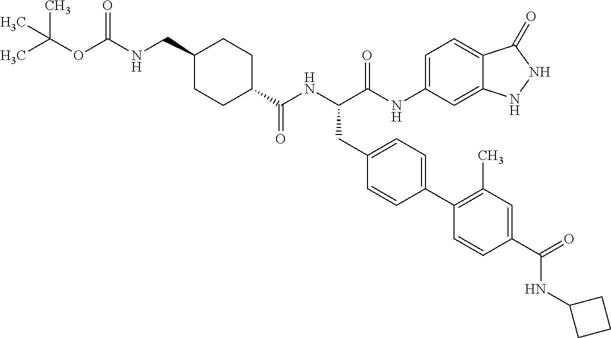 [SMILES: Cc1cc(C(=O)NC2CCC2)ccc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

A solution of (2S)-2-{[(trans-4-{[(tert-butoxycarbonyl)amino]methyl}cyclohexyl)-carbonyl]amino}-3-[4′-(cyclobutylcarbamoyl)-2′-methylbiphenyl-4-yl]propanoic acid (80 mg, 0.14 mmol) in DMF (1.5 ml) was admixed with 6-amino-1,2-dihydro-3H-indazol-3-one (40 mg, 0.27 mmol) and N,N-diisopropylethylamine (0.07 ml, 0.41 mmol). The solution was admixed with HATU (77 mg, 0.41 mmol) and then stirred at RT overnight. The solvent was removed and the residue was dissolved in a little DMSO/acetonitrile, filtered through a Millipore filter and purified by preparative HPLC (eluent: gradient of acetonitrile/water with 0.1% trifluoroacetic acid). This gave 28 mg (29% of theory) of the title compound.

LC-MS (Method 1): Rₜ=0.99 min; MS (ESIneg): m/z=721 [M−H]⁻.

### Example 271A

### tert-Butyl [(trans-4-{[(2S)-1-(1H-indazol-6-ylamino)-3-{2′-methyl-4′-[(6-oxohexahydropyrrolo[3,4-b]pyrrol-1(2H)-yl)carbonyl]biphenyl-4-yl}-1-oxopropan-2-yl]carbamoyl}-cyclohexyl)methyl]carbamate

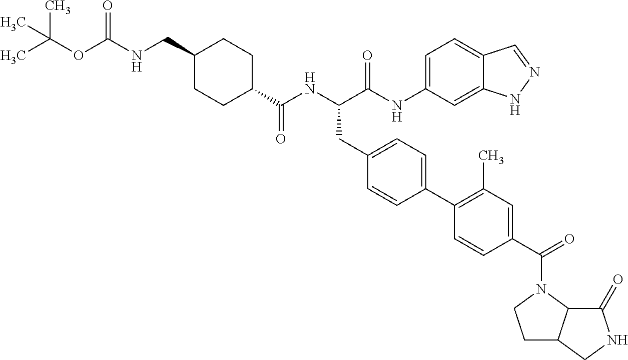 [SMILES: Cc1cc(C(=O)N2CCC3CNC(=O)C32)ccc1-c1ccc(C[C@H](NC(=O)[C@H]2CC[C@H](CNC(=O)OC(C)(C)C)CC2)C(=O)Nc2ccc3cn[nH]c3c2)cc1]

A solution of 4′-[(2S)-2-{[(trans-4-{[(tert-butoxycarbonyl)amino]methyl}cyclohexyl)-carbonyl]amino}-3-(1H-indazol-6-ylamino)-3-oxopropyl]-2-methylbiphenyl-4-carboxylic acid (100 mg, 0.15 mmol) in DMF (1.25 ml) was admixed with hexahydropyrrolo[3,4-b]pyrrol-6(1H)-one (126 mg, 0.31 mmol) and N,N-diisopropylethylamine (0.08 ml, 0.46 mmol). The solution was admixed with HATU (87 mg, 0.23 mmol) and then stirred at RT overnight. The residue was diluted with water and acetonitrile, filtered through a Millipore filter and purified by preparative HPLC (eluent: gradient of acetonitrile/water with 0.1% trifluoroacetic acid). This gave 61 mg (52% of theory) of the title compound.

LC-MS (Method 1): Rₜ=0.92 min; MS (ESIneg): m/z=760 [M−H]⁻.

### Example 272A

### tert-Butyl [(trans-4-{[(2S)-3-{4′-[(3-hydroxycyclobutyl)carbamoyl]-2′-methylbiphenyl-4-yl}-1-(1H-indazol-6-ylamino)-1-oxopropan-2-yl]carbamoyl}cyclohexyl)methyl]carbamate

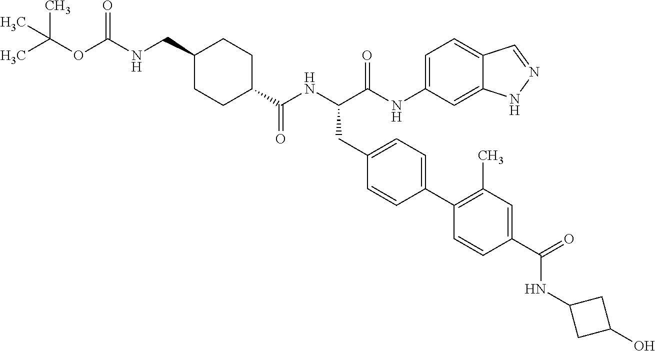 [SMILES: Cc1cc(C(=O)NC2CC(O)C2)ccc1-c1ccc(C[C@H](NC(=O)[C@H]2CC[C@H](CNC(=O)OC(C)(C)C)CC2)C(=O)Nc2ccc3cn[nH]c3c2)cc1]

A solution of 4′-[(2S)-2-{[(trans-4-{[(tert-butoxycarbonyl)amino]methyl}cyclohexyl)-carbonyl]amino}-3-(1H-indazol-6-ylamino)-3-oxopropyl]-2-methylbiphenyl-4-carboxylic acid (100 mg, 0.15 mmol) in DMF (1.25 ml) was admixed with 3-aminocyclobutanol (38 mg, 0.31 mmol) and N,N-diisopropylethylamine (0.11 ml, 0.61 mmol). The solution was admixed with HATU (87 mg, 0.23 mmol) and then stirred at RT overnight. The residue was diluted with water and acetonitrile, filtered through a Millipore filter and purified by preparative HPLC (eluent: gradient of acetonitrile/water with 0.1% trifluoroacetic acid). This gave 92.3 mg (83% of theory) of the title compound.

LC-MS (Method 1): Rₜ=0.92 min; MS (ESIneg): m/z=721 [M−H]⁻.

### Example 273A

### tert-Butyl {[trans-4-({(2S)-3-[4′-(isopropylcarbamoyl)-2′-methylbiphenyl-4-yl]-1-oxo-1-[(4-{5-[1,1,2,2-tetrafluoro-3-(methylamino)-3-oxopropyl]-4H-1,2,4-triazol-3-yl}phenyl)amino]propan-2-yl}carbamoyl)cyclohexyl]methyl}carbamate

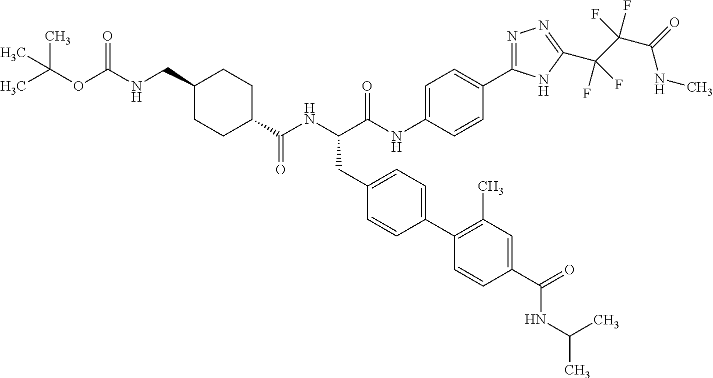 [SMILES: CNC(=O)C(F)(F)C(F)(F)c1nnc(-c2ccc(NC(=O)[C@H](Cc3ccc(-c4ccc(C(=O)NC(C)C)cc4C)cc3)NC(=O)[C@H]3CC[C@H](CNC(=O)OC(C)(C)C)CC3)cc2)[nH]1]

A suspension of (2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoic acid (100 mg, 0.172 mmol) in ethyl acetate (3 ml) was admixed with 3-[5-(4-aminophenyl)-4H-1,2,4-triazol-3-yl]-2,2,3,3-tetrafluoro-N-methylpropanamide (60 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.3 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 89.1 mg (56% of theory) of the title compound.

LC-MS (Method 1): Rₜ=1.05 min; MS (ESIneg): m/z=877 [M−H]⁻.

### Example 274A

### 3-[5-(4-{[(2S)-2-{[(trans-4-{[(tert-Butoxycarbonyl)amino]methyl}cyclohexyl)carbonyl]amino}-3-(2′-methyl-4′-{[(3R)-2-oxopiperidin-3-yl]carbamoyl}biphenyl-4-yl)propanoyl]amino}phenyl)-4H-1,2,4-triazol-3-yl]-2,2,3,3-tetrafluoropropanoic acid

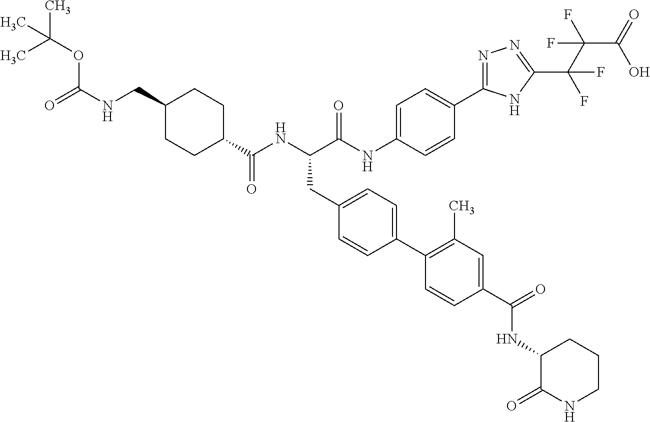 [SMILES: Cc1cc(C(=O)N[C@@H]2CCCNC2=O)ccc1-c1ccc(C[C@H](NC(=O)[C@H]2CC[C@H](CNC(=O)OC(C)(C)C)CC2)C(=O)Nc2ccc(-c3nnc(C(F)(F)C(F)(F)C(=O)O)[nH]3)cc2)cc1]

4′-[(2S)-2-{[(trans-4-{[(tert-butoxycarbonyl)amino]methyl}cyclohexyl)carbonyl]amino}-3-({4-[5-(2-carboxy-1,1,2,2-tetrafluoroethyl)-4H-1,2,4-triazol-3-yl]phenyl}amino)-3-oxopropyl]-2-methylbiphenyl-4-carboxylic acid (100 mg, 0.12 mmol) and (3R)-3-aminopiperidin-2-one (17 mg, 0.15 mmol) were dissolved in 1 ml of N,N-dimethylformamide, admixed with N,N-diisopropylethylamine (84 μl, 0.49 mmol) and O-(7-azabenzotriazol-1-yl)-N,N,N′N′-tetramethyluronium hexafluorophosphate (126 μl, 0.18 mmol) and stirred at RT for 18 h. The mixture was filtered through a Millipore filter and purified via preparative HPLC (eluent: acetonitrile/water with 0.1% trifluoroacetic acid (gradient)). This gave 67 mg (56% of theory) of the title compound.

LC-MS (Method 1): Rₜ=0.88 min; MS (ESIpos): m/z=921.5 [M+H]⁺.

### Example 275A

### tert-Butyl [(trans-4-{[(2S)-3-[4′-(cyclopropylcarbamoyl)-2′-methylbiphenyl-4-yl]-1-(1H-indazol-6-ylamino)-1-oxopropan-2-yl]carbamoyl}cyclohexyl)methyl]carbamate

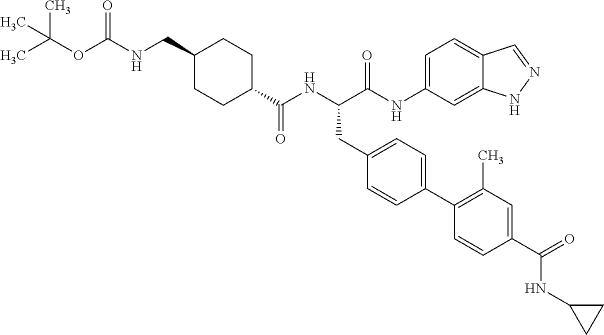 [SMILES: Cc1cc(C(=O)NC2CC2)ccc1-c1ccc(C[C@H](NC(=O)[C@H]2CC[C@H](CNC(=O)OC(C)(C)C)CC2)C(=O)Nc2ccc3cn[nH]c3c2)cc1]

A solution of 4′-[(2S)-2-{[(trans-4-{[(tert-butoxycarbonyl)amino]methyl}cyclohexyl)-carbonyl]amino}-3-(1H-indazol-6-ylamino)-3-oxopropyl]-2-methylbiphenyl-4-carboxylic acid (80 mg, 0.12 mmol) in DMF (1 ml) was admixed with cyclopropylamine (0.02 ml, 0.25 mmol) and N,N-diisopropylethylamine (0.064 ml, 0.37 mmol). The solution was admixed with HATU (70 mg, 0.18 mmol) and stirred at RT overnight. The residue was admixed with a little water and acetonitrile, filtered through a Millipore filter and purified by preparative HPLC (eluent: gradient of acetonitrile/water with 0.1% trifluoroacetic acid). This gave 56.7 mg (67% of theory) of the title compound.

LC-MS (Method 1): Rₜ=1.00 min; MS (ESIneg): m/z=691 [M−H]⁻.

### Example 276A

### tert-Butyl {[trans-4-({(2S)-1-[(7-chloro-2-oxo-2,3-dihydro-1,3-benzoxazol-5-yl)amino]-3-[4′-(cyclobutylcarbamoyl)-2′-methylbiphenyl-4-yl]-1-oxopropan-2-yl}carbamoyl)cyclohexyl]-methyl}carbamate

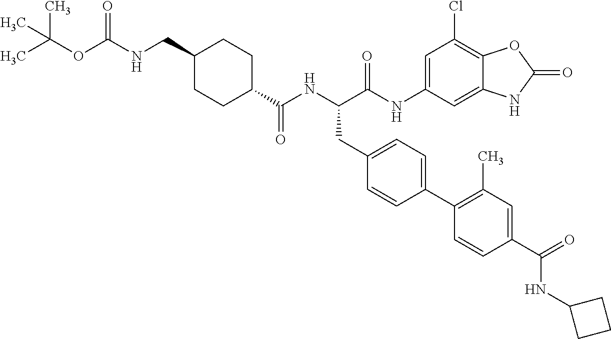 [SMILES: Cc1cc(C(=O)NC2CCC2)ccc1-c1ccc(C[C@H](NC(=O)[C@H]2CC[C@H](CNC(=O)OC(C)(C)C)CC2)C(=O)Nc2cc(Cl)c3oc(=O)[nH]c3c2)cc1]

A suspension of (2S)-2-{[(trans-4-{[(tert-butoxycarbonyl)amino]methyl}cyclohexyl)-carbonyl]amino}-3-[4′-(cyclobutylcarbamoyl)-2′-methylbiphenyl-4-yl]propanoic acid (80 mg, 0.14 mmol) in ethyl acetate (2 ml) was admixed with 5-amino-7-chloro-1,3-benzoxazol-2(3H)-one (27.4 mg, 0.15 mmol) and N,N-diisopropylethylamine (0.07 ml, 0.41 mmol). The suspension was admixed with a 2,4,6-tripropyl-1,3,5,2,4,6-trioxatriphosphinane 2,4,6-trioxide solution (500 in DMF, 0.24 ml, 0.41 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 30 mg (29% of theory) of the title compound.

LC-MS (Method 1): Rₜ=1.13 min; MS (ESIneg): m/z=756 [M−H]⁻.

### Example 277A

### 2,2,3,3,4,4-Hexafluoro-4-[5-(4-nitrophenyl)-4H-1,2,4-triazol-3-yl]butanoic acid

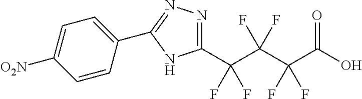 [SMILES: O=C(O)C(F)(F)C(F)(F)C(F)(F)c1nnc(-c2ccc([N+](=O)[O-])cc2)[nH]1]

A solution of 4-nitrobenzenecarboximidohydrazide (900 mg, 5.0 mmol) in dichloromethane (20 ml) was admixed with 3,3,4,4,5,5-hexafluorodihydro-2H-pyran-2,6(3H)-dione (2.0 ml, 15.0 mmol), then admixed with acetonitrile (20 ml) and stirred at 50° C. for 3 h, then stirred at RT overnight. The reaction mixture was stirred at 90° C. for another 4 h and, after addition of 4 Å molecular sieve, stirred at RT for a further 4 days. The solvent was removed on a rotary evaporator and the residue was diluted with water and acetonitrile, filtered through a Millipore filter and purified by preparative HPLC (eluent: gradient of acetonitrile/water with 0.1% trifluoroacetic acid). This gave 1.49 g (77% of theory) of the title compound.

LC-MS (Method 1): Rₜ=0.69 min; MS (ESIneg): m/z=383 [M−H]⁻.

### Example 278A

### 4-[5-(4-Aminophenyl)-4H-1,2,4-triazol-3-yl]-2,2,3,3,4,4-hexafluorobutanoic acid hydrochloride

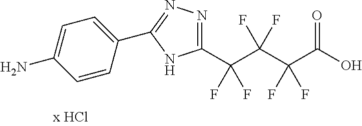 [SMILES: Nc1ccc(-c2nnc(C(F)(F)C(F)(F)C(F)(F)C(=O)O)[nH]2)cc1]

2,2,3,3,4,4-Hexafluoro-4-[5-(4-nitrophenyl)-4H-1,2,4-triazol-3-yl]butanoic acid (4.21 g, 10.96 mmol) and tin(II) chloride hydrate (9.89 g, 43.8 mmol) were stirred in ethanol (70 ml) at 70° C. for 1 h. The reaction mixture was poured onto ice-water and adjusted to pH 8 with solid sodium carbonate. The salts were filtered out of the mixture and washed with ethyl acetate. The aqueous phase was acidified with 1N hydrochloric acid and the solvent was removed on a rotary evaporator. The residue was stirred with acetone and a little methanol and filtered with suction. The residue was dried under high vacuum. This gave 3.59 g (76% of theory) of the title compound.

LC-MS (Method 1): Rₜ=0.44 min; MS (ESIneg): m/z=353 [M−H—HCl]⁻.

### Example 279A

### Methyl 3-[5-(4-{[(2S)-2-({[trans-4-(aminomethyl)cyclohexyl]carbonyl}amino)-3-(2′-methyl-4′-{[trans-4-(2,2,2-trifluoroacetoxy)cyclohexyl]carbamoyl}biphenyl-4-yl)propanoyl]amino}phenyl)-4H-1,2,4-triazol-3-yl]-2,2,3,3-tetrafluoropropanoate trifluoroacetate (enantiomer 1)

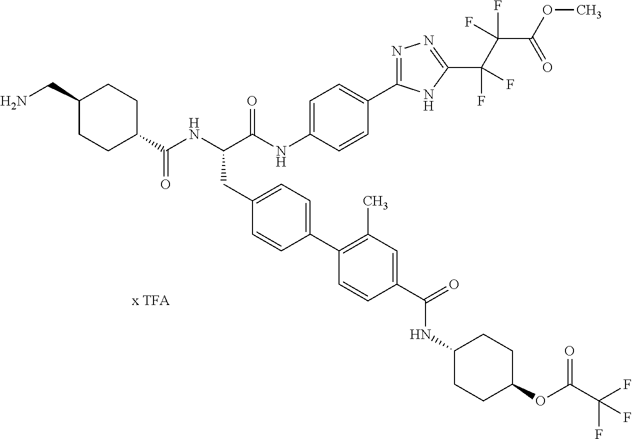 [SMILES: COC(=O)C(F)(F)C(F)(F)c1nnc(-c2ccc(NC(=O)[C@H](Cc3ccc(-c4ccc(C(=O)N[C@H]5CC[C@H](OC(=O)C(F)(F)F)CC5)cc4C)cc3)NC(=O)[C@H]3CC[C@H](CN)CC3)cc2)[nH]1]

Methyl 3-[5-(4-{[(2S)-2-{[(trans-4-{[(tert-butoxycarbonyl)amino]methyl}cyclohexyl)carbonyl]-amino}-3-{4′-[(trans-4-{[tert-butyl(dimethyl)silyl]oxy}cyclohexyl)carbamoyl]-2′-methylbiphenyl-4-yl}propanoyl]amino}phenyl)-4H-1,2,4-triazol-3-yl]-2,2,3,3-tetrafluoropropanoate (enantiomer 1) (3.60 g, 3.43 mmol) was added to 36.00 ml of trifluoroacetic acid and the solution was stirred at RT overnight. Subsequently, the reaction mixture was concentrated, and the residue was stirred with 100 ml of diethyl ether, filtered, washed through three times with 50 ml each time of diethyl ether and dried under high vacuum. 3.21 g (85% of theory) of the title compound were obtained.

¹H NMR (400 MHz, DMSO-d₆): δ=15.18 (br. s., 1H), 10.42 (s, 1H), 8.27-8.18 (m, 2H), 7.96 (d, 2H), 7.83-7.54 (m, 7H), 7.38 (d, 2H), 7.29-7.19 (m, 2H), 4.99-4.89 (m, 1H), 4.79-4.70 (m, 1H), 3.95 (s, 3H), 3.91-3.79 (m, 1H), 3.18-3.08 (m, 1H), 2.99-2.89 (m, 1H), 2.69-2.61 (m, 2H), 2.23 (s, 3H), 2.17-2.04 (m, 3H), 1.98-1.88 (m, 2H), 1.81-1.38 (m, 9H), 1.35-1.12 (m, 2H), 1.00-0.84 (m, 2H).

LC-MS (Method 1): Rₜ=0.89 min; MS (ESIneg): m/z=932 [M−H—C₂HF₃O₂]⁻.

### WORKING EXAMPLES

### Example 1

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-(piperidin-4-yl)biphenyl-4-carboxamide hydrochloride

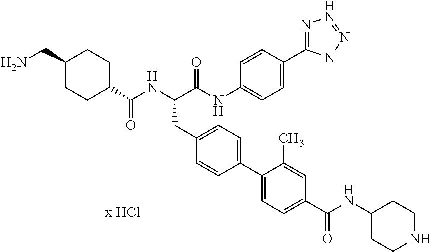

To a solution of 79 mg (81 μmol) of tert-butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]piperidine-1-carboxylate in 3 ml of dioxane was added 0.3 ml (1.2 mmol) of 4M hydrogen chloride in dioxane. The mixture was stirred at RT overnight. The precipitated solid was filtered off with suction, washed with acetonitrile and dried under high vacuum. 63 mg (99% of theory, 93% purity) of the title compound were obtained.

¹H NMR (400 MHz, DMSO-d₆): δ=0.83-1.00 (m, 2H), 1.11-1.35 (m, 2H), 1.40-1.63 (m, 2H), 1.66-1.85 (m, 5H), 1.90-2.02 (m, 2H), 2.10-2.18 (m, 4H), 2.57-2.70 (m, 2H), 2.89-3.08 (m, 3H), 3.14 (dd, 1H), 3.25-3.41 (m, 2H), 4.01-4.13 (m, 1H), 4.70-4.80 (m, 1H), 7.20-7.30 (m, 3H), 7.40 (d, 2H), 7.69-7.91 (m, 7H), 8.02 (d, 2H), 8.30 (d, 1H), 8.49 (d, 1H), 8.64-8.87 (m, 2H), 10.55 (s, 1H), 16.8 (bs, 1H).

LC-MS (Method 1): Rₜ=0.52 min; MS (ESIneg): m/z=622 [M−H—HCl]⁻.

### Example 2

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[2-(diethylamino)ethyl]-2-methylbiphenyl-4-carboxamide hydrochloride

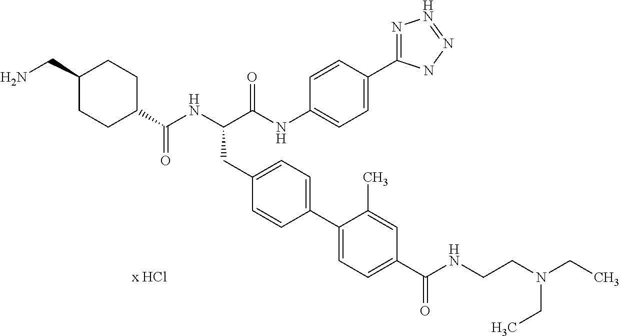

To a solution of 70 mg (78 μmol) of tert-butyl [(trans-4-{[(2S)-3-(4′-{[2-(diethylamino)ethyl]-carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate in 2.5 ml of dioxane was added 0.3 ml (1.2 mmol) of 4M hydrogen chloride in dioxane. The mixture was stirred at RT overnight. The precipitated solid was filtered off with suction, washed with acetonitrile and dried under high vacuum. 53 mg (87% of theory) of the title compound were obtained.

¹H NMR (400 MHz, DMSO-d₆): δ=0.85-0.99 (m, 2H), 1.12-1.33 (m, 8H), 1.40-1.62 (m, 2H), 1.68-1.82 (m, 3H), 2.10-2.21 (m, 1H), 2.22 (s, 3H), 2.58-2.68 (m, 2H), 2.96 (dd, 1H), 2.99-3.27 (m, 7H), 3.60-3.68 (m, 2H), 4.71-4.79 (m, 1H), 7.23-7.30 (m, 3H), 7.40 (d, 2H), 7.74-7.87 (m, 7H), 8.02 (d, 2H), 8.29 (d, 1H), 8.88 (t, 1H), 10.0 (bs, 1H), 10.54 (s, 1H).

LC-MS (Method 1): Rₜ=0.63 min; MS (ESIneg): m/z=678 [M−H—HCl]⁻.

### Example 3

### N-(2-Aminoethyl)-4′-[(2S)-2-({[trans-4-(aminomethyl)cyclohexyl]carbonyl}amino)-3-(1H-indazol-6-ylamino)-3-oxopropyl]-2-methylbiphenyl-4-carboxamide hydrochloride

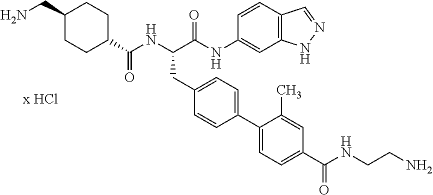

To a solution of 76 mg (84 μmol) of tert-butyl [(trans-4-{[(2S)-3-[4′-{(2-[(tert-butoxycarbonyl)-amino]ethyl}carbamoyl)-2′-methylbiphenyl-4-yl]-1-(1H-indazol-6-ylamino)-1-oxopropan-2-yl]carbamoyl}cyclohexyl)methyl]carbamate trifluoroacetate in 3 ml of dioxane was added 0.32 ml (1.25 mmol) of 4M hydrogen chloride in dioxane. The mixture was stirred at RT overnight. The precipitated solid was filtered off with suction, washed with acetonitrile and dried under high vacuum. 97 mg (87% of theory, 91% purity) of the title compound were obtained.

¹H NMR (400 MHz, DMSO-d₆): δ=0.85-1.00 (m, 2H), 1.10-1.33 (m, 2H), 1.41-1.61 (m, 2H), 1.67-1.82 (m, 3H), 2.11-2.27 (m, 4H), 2.58-2.68 (m, 2H), 2.90-3.04 (m, 3H), 3.12 (dd, 1H), 3.46-3.60 (m, 2H), 4.71-4.80 (m, 1H), 7.12 (d, 1H), 7.20-7.29 (m, 3H), 7.39 (d, 2H), 7.66 (d, 1H), 7.70-7.90 (m, 5H), 7.91-8.10 (m, 4H), 8.02 (s, 1H), 8.28 (d, 1H), 8.73 (t, 1H), 10.36 (s, 1H), 12.9 (s, 1H).

LC-MS (Method 1): Rₜ=0.60 min; MS (ESIneg): m/z=594 [M−H—HCl]⁻.

### Example 4

### 4′-[(2S)-2-({[trans-4-(Aminomethyl)cyclohexyl]carbonyl}amino)-3-(1H-indazol-6-ylamino)-3-oxopropyl]-2-methyl-N-(piperidin-4-yl)biphenyl-4-carboxamide hydrochloride

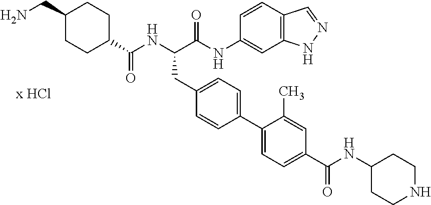

To a solution of 84 mg (88 μmol) of tert-butyl 4-[({4′-[(2S)-2-{[(trans-4-{[(tert-butoxycarbonyl)amino]methyl}cyclohexyl)-carbonyl]amino}-3-(1H-indazol-6-ylamino)-3-oxopropyl]-2-methylbiphenyl-4-yl}carbonyl)amino]piperidine-1-carboxylate trifluoroacetate in 3 ml of dioxane was added 0.33 ml (1.33 mmol) of 4M hydrogen chloride in dioxane. The mixture was stirred at RT overnight. The precipitated solid was filtered off with suction, washed with acetonitrile and dried under high vacuum. 72 mg (quant.) of the title compound were obtained.

¹H NMR (400 MHz, DMSO-d₆): δ=0.85-0.99 (m, 2H), 1.10-1.33 (m, 2H), 1.40-1.61 (m, 2H), 1.67-1.85 (m, 5H), 1.90-2.00 (m, 2H), 2.10-2.28 (m, 4H), 2.58-2.69 (m, 2H), 2.92-3.08 (m, 3H), 3.12 (dd, 1H), 3.26-3.38 (m, 2H), 4.01-4.14 (m, 1H), 4.72-4.81 (m, 1H), 7.15 (d, 1H), 7.20-7.29 (m, 3H), 7.40 (d, 2H), 7.67 (d, 1H), 7.73 (d, 1H), 7.80 (s, 1H), 7.81-7.95 (m, 3H), 7.97 (s, 1H), 8.13 (s, 1H), 8.28 (d, 1H), 8.50 (d, 1H), 8.73-8.90 (m, 2H), 10.37 (s, 1H), 12.9 (s, 1H).

LC-MS (Method 1): Rₜ=0.61 min; MS (ESIneg): m/z=634 [M−H—HCl]⁻.

### Example 5

### trans-4-(Aminomethyl)-N-{(2S)-1-(1H-indazol-6-ylamino)-3-[2′-methyl-4′-(piperazin-1-ylcarbonyl)biphenyl-4-yl]-1-oxopropan-2-yl}cyclohexanecarboxamide hydrochloride

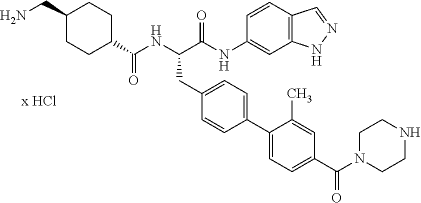

To a solution of 98 mg (105 μmol) of tert-butyl 4-({4′-[(2S)-2-{[(trans-{[(tert-butoxycarbonyl)amino]methyl}cyclohexyl)-carbonyl]amino}-3-(1H-indazol-6-ylamino)-3-oxopropyl]-2-methylbiphenyl-4-yl}carbonyl)piperazine-1-carboxylate trifluoroacetate in 3 ml of dioxane was added 0.39 ml (1.57 mmol) of 4M hydrogen chloride in dioxane. The mixture was stirred at RT overnight. The precipitated solid was filtered off with suction, washed with acetonitrile and dried under high vacuum. 73 mg (95% of theory) of the title compound were obtained.

¹H NMR (400 MHz, DMSO-d₆): δ=0.85-1.00 (m, 2H), 1.10-1.34 (m, 2H), 1.40-1.61 (m, 2H), 1.68-1.82 (m, 3H), 2.11-2.26 (m, 4H), 2.57-2.69 (m, 2H), 2.97 (dd, 1H), 3.03-3.25 (m, 5H), 3.55-3.90 (m, 4H), 4.73-4.81 (m, 1H), 7.13 (d, 1H), 7.19-7.30 (m, 3H), 7.30-7.43 (m, 4H), 7.67 (d, 1H), 7.75-7.95 (m, 3H), 7.96 (s, 1H), 8.13 (s, 1H), 8.31 (d, 1H), 9.4 (bs, 2H), 10.37 (s, 1H), 12.9 (s, 1H).

LC-MS (Method 1): Rₜ=0.59 min; MS (ESIneg): m/z=620 [M−H—HCl]⁻.

### Example 6

### 4′-[(2S)-2-({[trans-4-(Aminomethyl)cyclohexyl]carbonyl}amino)-3-(1H-indazol-6-ylamino)-3-oxopropyl]-N-[2-(diethylamino)ethyl]-2-methylbiphenyl-4-carboxamide hydrochloride

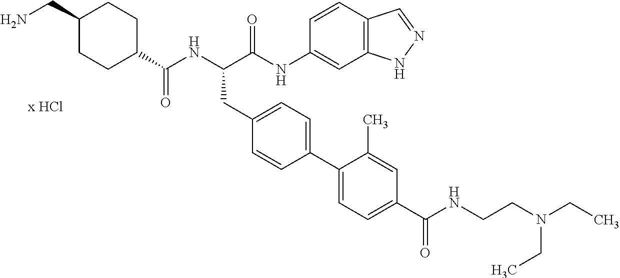

To a solution of 57 mg (66 μmol) of tert-butyl [(trans-4-{[(2S)-3-(4′-{[2-(diethylamino)ethyl]carbamoyl}-2′-methylbiphenyl-4-yl)-1-(1H-indazol-6-ylamino)-1-oxopropan-2-yl]carbamoyl}cyclohexyl)methyl]carbamate trifluoroacetate in 2.5 ml of dioxane was added 0.25 ml (0.99 mmol) of 4M hydrogen chloride in dioxane. The mixture was stirred at RT overnight. The precipitated solid was filtered off with suction, washed with acetonitrile and dried under high vacuum. 45 mg (92% of theory) of the title compound were obtained.

¹H NMR (400 MHz, DMSO-d₆): δ=0.85-1.00 (m, 2H), 1.11-1.34 (m, 8H), 1.42-1.62 (m, 2H), 1.70-1.82 (m, 3H), 2.11-2.26 (m, 4H), 2.58-2.69 (m, 2H), 2.96 (dd, 1H), 3.10-3.27 (m, 6H), 3.62-3.68 (m, 2H), 4.72-4.82 (m, 1H), 7.14 (d, 1H), 7.23-7.29 (m, 3H), 7.40 (d, 2H), 7.67 (d, 1H), 7.75-7.94 (m, 4H), 7.97 (s, 1H), 8.13 (s, 1H), 8.28 (d, 1H), 8.92 (t, 1H), 10.2 (bs, 1H), 10.38 (s, 1H), 12.9 (s, 1H).

LC-MS (Method 1): Rₜ=0.65 min; MS (ESIneg): m/z=650 [M−H—HCl]⁻.

### Example 7

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-isopropyl-2-methylbiphenyl-4-carboxamide hydrochloride

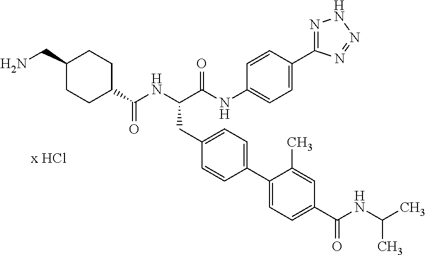

To a solution of 45 mg (54 μmol) of ten-butyl [(trans-4-{[(2S)-3-(4′-{[2-(diethylamino)ethyl]carbamoyl}-2′-methylbiphenyl-4-yl)-1-(1H-indazol-6-ylamino)-1-oxopropan-2-yl]carbamoyl}cyclohexyl)methyl]carbamate in 2.0 ml of dioxane was added 0.20 ml (0.81 mmol) of 4M hydrogen chloride in dioxane. The mixture was then stirred at RT for 48 h. The precipitated solid was filtered off with suction, washed with acetonitrile and dried under high vacuum. 38 mg (91% of theory, 90% purity) of the title compound were obtained.

¹H NMR (400 MHz, DMSO-d₆): δ=0.85-0.98 (m, 2H), 1.10-1.35 (m, 8H), 1.40-1.62 (m, 2H), 1.68-1.81 (m, 3H), 2.11-2.26 (m, 4H), 2.58-2.69 (m, 2H), 2.95 (dd, 1H), 3.13 (dd, 1H), 4.05-4.15 (m, 1H), 4.70-4.79 (m, 1H), 7.21 (d, 1H), 7.26 (d, 2H), 7.40 (d, 2H), 7.66-7.87 (m, 6H), 8.02 (d, 2H), 8.21 (d, 1H), 8.28 (d, 1H), 10.53 (s, 1H).

LC-MS (Method 1): Rₜ=0.77 min; MS (ESIneg): m/z=621 [M−H—HCl]⁻.

### Example 8

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-[(3R)-pyrrolidin-3-yl]biphenyl-4-carboxamide hydrochloride

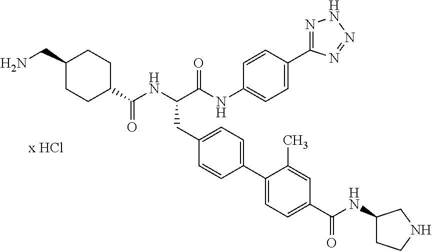

To a solution of 58 mg (60 μmol) of tert-butyl (3R)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}carbonyl)amino]pyrrolidine-1-carboxylate in 2.5 ml of dioxane was added 0.23 ml (0.90 mmol) of 4M hydrogen chloride in dioxane. The mixture was then stirred at RT for 48 h. The precipitated solid was filtered off with suction, washed with acetonitrile and dried under high vacuum. 44 mg (97% of theory) of the title compound were obtained.

¹H NMR (400 MHz, DMSO-d₆): δ=0.85-0.99 (m, 2H), 1.12-1.35 (m, 2H), 1.42-1.64 (m, 2H), 1.69-1.82 (m, 4H), 1.95-2.07 (m, 1H), 2.11-2.28 (m, 5H), 2.60-2.70 (m, 2H), 2.96 (dd, 1H), 3.09-3.34 (m, 4H), 4.52-4.59 (m, 1H), 4.71-4.79 (m, 1H), 7.23-7.28 (m, 3H), 7.40 (d, 2H), 7.72-7.87 (m, 6H), 8.02 (d, 2H), 8.29 (d, 1H), 8.72 (d, 1H), 9.1 (bs, 1H), 9.3 (bs, 1H), 10.54 (s, 1H), 16.8 (bs, 1H).

LC-MS (Method 1): Rₜ=0.60 min; MS (ESIneg): m/z=648 [M−H—HCl]⁻.

### Example 9

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-[(3S)-pyrrolidin-3-yl]biphenyl-4-carboxamide hydrochloride

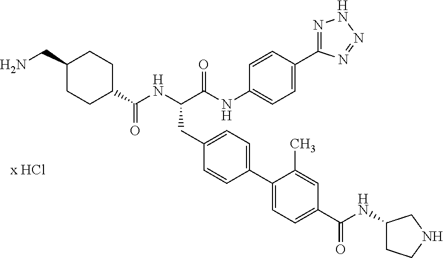

To a solution of 56 mg (58 μmol) of tert-butyl (3S)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}carbonyl)amino]pyrrolidine-1-carboxylate in 2.5 ml of dioxane was added 0.22 ml (0.87 mmol) of 4M hydrogen chloride in dioxane. The mixture was stirred at RT for 48 h. The precipitated solid was filtered off with suction, washed with acetonitrile and dried under high vacuum. 43 mg (97% of theory) of the title compound were obtained.

¹H NMR (400 MHz, DMSO-d₆): δ=0.85-0.99 (m, 2H), 1.12-1.35 (m, 2H), 1.42-1.64 (m, 2H), 1.69-1.82 (m, 4H), 1.95-2.07 (m, 1H), 2.11-2.28 (m, 5H), 2.60-2.70 (m, 2H), 2.96 (dd, 1H), 3.09-3.34 (m, 4H), 4.52-4.59 (m, 1H), 4.71-4.79 (m, 1H), 7.23-7.28 (m, 3H), 7.40 (d, 2H), 7.72-7.87 (m, 6H), 8.02 (d, 2H), 8.29 (d, 1H), 8.72 (d, 1H), 9.1 (bs, 1H), 9.3 (bs, 1H), 10.54 (s, 1H), 16.8 (bs, 1H).

LC-MS (Method 1): Rₜ=0.63 min; MS (ESIneg): m/z=648 [M−H—HCl]⁻.

### Example 10

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-N-[2-(diethylamino)ethyl]biphenyl-4-carboxamide hydrochloride

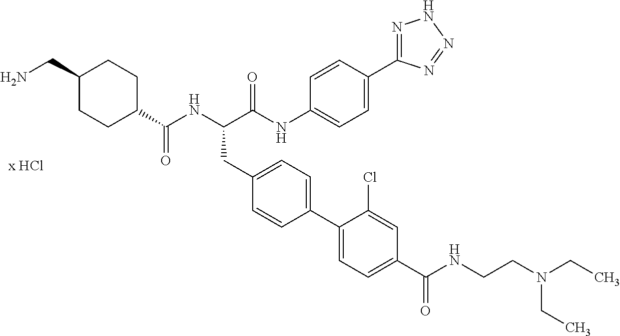

To a solution of 72 mg (79 μmol) of tert-butyl [(trans-4-{[(2S)-3-(2′-chloro-4′-{[2-(diethylamino)ethyl]carbamoyl}biphenyl-4-yl)-1-oxo-1-{[4-(2H-tetrazol-5-yl)phenyl]amino}-propan-2-yl]carbamoyl}cyclohexyl)methyl]carbamate trifluoroacetate in 2.5 ml of dioxane was added 0.30 ml (1.18 mmol) of 4M hydrogen chloride in dioxane. The mixture was stirred at RT overnight. The precipitated solid was filtered off with suction, washed with acetonitrile and dried under high vacuum. 61 mg (94% of theory) of the title compound were obtained.

¹H NMR (400 MHz, DMSO-d₆): δ=0.85-1.00 (m, 2H), 1.13-1.34 (m, 8H), 1.42-1.64 (m, 2H), 1.68-1.83 (m, 3H), 2.11-2.21 (m, 1H), 2.59-2.69 (m, 2H), 2.97 (dd, 1H), 3.12-3.29 (m, 7H), 3.62-3.69 (m, 2H), 4.72-4.80 (m, 1H), 7.38 (d, 2H), 7.43 (d, 2H), 7.49 (d, 1H), 7.75-7.88 (m, 5H), 7.93 (d, 1H), 8.02 (d, 2H), 8.08 (s, 1H), 8.29 (d, 1H), 9.08 (t, 1H), 10.1 (bs, 1H), 10.58 (s, 1H).

LC-MS (Method 1): Rₜ=0.60 min; MS (ESIneg): m/z=698 [M−H—HCl]⁻.

### Example 11

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-N-(piperidin-4-yl)biphenyl-4-carboxamide hydrochloride

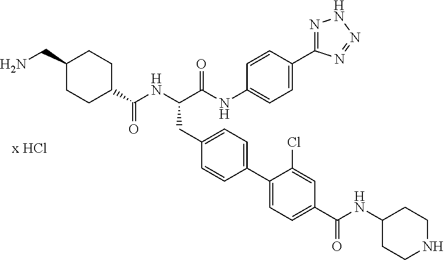

To a solution of 65 mg (65 μmol) of tert-butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-2-chlorobiphenyl-4-yl}carbonyl)amino]piperidine-1-carboxylate in 2.5 ml of dioxane was added 0.24 ml (0.98 mmol) of 4M hydrogen chloride in dioxane. The mixture was stirred at RT overnight. The precipitated solid was filtered off with suction, washed with acetonitrile and dried under high vacuum. 54 mg (quant.) of the title compound were obtained.

¹H NMR (400 MHz, DMSO-d₆): δ=0.85-0.98 (m, 2H), 1.14-1.33 (m, 2H), 1.40-1.65 (m, 2H), 1.66-1.85 (m, 4H), 1.93-2.02 (m, 2H), 2.10-2.20 (m, 1H), 2.57-2.69 (m, 2H), 2.90-3.09 (m, 3H), 3.14 (dd, 1H), 3.23-3.50 (m, 4H), 4.01-4.13 (m, 1H), 4.70-4.80 (m, 1H), 7.32-7.49 (m, 5H), 7.70-7.87 (m, 5H), 7.88 (d, 1H), 7.96-8.05 (m, 2H), 8.29 (d, 1H), 8.60-8.82 (m, 3H), 10.56 (s, 1H), 16.8 (bs, 1H).

LC-MS (Method 1): Rₜ=0.62 min; MS (ESIneg): m/z=682 [M−H—HCl]⁻.

### Example 12

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-N-[(3R)-pyrrolidin-3-yl]biphenyl-4-carboxamide hydrochloride

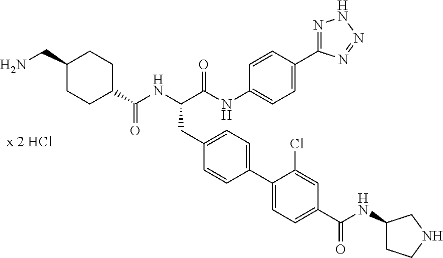

To a solution of 87 mg (88 μmol) of tert-butyl (3R)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-chlorobiphenyl-4-yl}carbonyl)amino]pyrrolidine-1-carboxylate in 2.5 ml of dioxane was added 0.33 ml (1.33 mmol) of 4M hydrogen chloride in dioxane. The mixture was stirred at RT overnight. The precipitated solid was filtered off with suction, washed with acetonitrile and dried under high vacuum. 70 mg (99% of theory, 93% purity) of the title compound were obtained.

¹H NMR (400 MHz, DMSO-d₆): δ=0.85-1.00 (m, 2H), 1.13-1.35 (m, 2H), 1.40-1.65 (m, 2H), 1.68-1.83 (m, 3H), 1.99-2.09 (m, 1H), 2.10-2.27 (m, 2H), 2.58-2.69 (m, 2H), 2.96 (dd, 1H), 3.10-3.32 (m, 3H), 3.32-3.53 (m, 2H), 4.50-4.60 (m, 1H), 4.71-4.80 (m, 1H), 7.32-7.52 (m, 5H), 7.70-7.87 (m, 4H), 7.92 (d, 1H), 8.02 (d, 2H), 8.08 (s, 1H), 8.30 (d, 1H), 8.90 (d, 1H), 9.1 (bs, 1H), 9.3 (bs, 1H), 10.57 (s, 1H).

LC-MS (Method 1): Rₜ=0.62 min; MS (ESIneg): m/z=668 [M−H—HCl]⁻.

### Example 13

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-N-[(3S)-pyrrolidin-3-yl]biphenyl-4-carboxamide hydrochloride

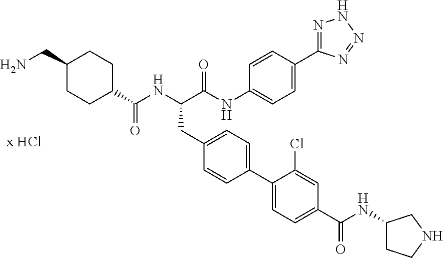

To a solution of 85 mg (86 μmol) of tert-butyl (3S)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-chlorobiphenyl-4-yl}carbonyl)amino]pyrrolidine-1-carboxylate in 2.5 ml of dioxane was added 0.32 ml (1.30 mmol) of 4M hydrogen chloride in dioxane. The mixture was stirred at RT overnight. The precipitated solid was filtered off with suction, washed with acetonitrile and dried under high vacuum. 66 mg (98% of theory) of the title compound were obtained.

¹H NMR (400 MHz, DMSO-d₆): δ=0.85-1.00 (m, 2H), 1.13-1.35 (m, 2H), 1.40-1.65 (m, 2H), 1.68-1.83 (m, 3H), 1.99-2.09 (m, 1H), 2.10-2.27 (m, 2H), 2.58-2.69 (m, 2H), 2.96 (dd, 1H), 3.10-3.32 (m, 3H), 3.32-3.53 (m, 2H), 4.50-4.60 (m, 1H), 4.71-4.80 (m, 1H), 7.32-7.52 (m, 5H), 7.70-7.87 (m, 4H), 7.92 (d, 1H), 8.02 (d, 2H), 8.08 (s, 1H), 8.30 (d, 1H), 8.90 (d, 1H), 9.1 (bs, 1H), 9.3 (bs, 1H), 10.57 (s, 1H).

LC-MS (Method 1): Rₜ=0.62 min; MS (ESIneg): m/z=668 [M−H—HCl]⁻.

### Example 14

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methylbiphenyl-4-carboxamide hydrochloride

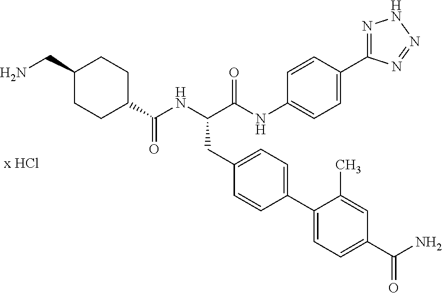

A solution of 50 mg (0.07 mmol) of tert-butyl [(trans-4-{[(2S)-3-(4′-carbamoyl-2′-methylbiphenyl-4-yl)-1-oxo-1-{[4-(2H-tetrazol-5-yl)phenyl]amino}propan-2-yl]carbamoyl}-cyclohexyl)methyl]carbamate in 2 ml of 1,4-dioxane was admixed with 0.28 ml (1.10 mmol) of 4M hydrogen chloride in 1,4-dioxane and stirred at RT for 3 h. The reaction mixture was separated directly by means of preparative HPLC (eluent: acetonitrile/water with 0.1% TFA (gradient)). The product-containing fractions were combined and concentrated on a rotary evaporator. The residue was dissolved in a little water/methanol/trifluoroacetic acid and separated once again by means of preparative HPLC (column: Sunfire C18, 5 μm, 250 mm×20 mm; eluent: water/acetonitrile/trifluoroacetic acid 69.95:30:0.05; flow rate: 25 ml/min; temperature: 40° C.; UV detection: 210 nm). The product-containing fractions were combined and admixed with a little 4M hydrogen chloride in 1,4-dioxane and the mixture was concentrated on a rotary evaporator. After lyophilization overnight, 12 mg (24% of theory, 97% purity) and 6 mg (9% of theory, 69% purity) of the title compound were obtained.

¹H NMR (400 MHz, DMSO-d₆): δ=0.85-0.99 (m, 2H), 1.10-1.34 (m, 2H), 1.40-1.62 (m, 2H), 1.68-1.81 (m, 3H), 2.05-2.27 (m, 4H), 2.58-2.69 (m, 2H), 2.95 (dd, 1H), 3.13 (dd, 1H), 4.71-4.80 (m, 1H), 7.18-7.41 (m, 5H), 7.65-7.85 (m, 7H), 7.92-8.04 (m, 3H), 8.26 (d, 1H), 10.50 (s, 1H), 16.8 (bs, 1H).

LC-MS (Method 1): Rₜ=0.67 min; MS (ESIneg): m/z=579 [M−H—HCl]⁻.

### Example 15

### N-(2-Aminoethyl)-4′-[(2S)-2-({[trans-4-(aminomethyl)cyclohexyl]carbonyl}amino)-3-oxo-3-{[4-(2H-tetrazol-5-yl)phenyl]amino}propyl]-2-methylbiphenyl-4-carboxamide hydrochloride

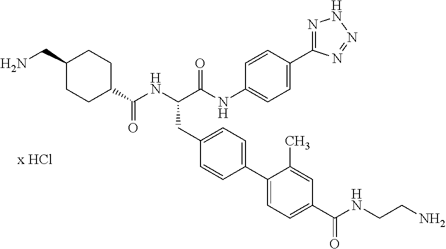

A solution of 93 mg (0.10 mmol) of tert-butyl [(trans-4-{[(2S)-3-[4′-{(2-[(tert-butoxycarbonyl)amino]ethyl}carbamoyl)-2′-methylbiphenyl-4-yl]-1-oxo-1-{[4-(2H-tetrazol-5-yl)phenyl]amino}propan-2-yl]carbamoyl}cyclohexyl)methyl]carbamate in 3 ml of 1,4-dioxane was admixed with 0.37 ml (1.49 mmol) of 4M hydrogen chloride in 1,4-dioxane and stirred at RT for 1.5 d. After the addition of 0.37 ml (1.49 mmol) of 4M hydrogen chloride in 1,4-dioxane, the mixture was stirred at RT for a further 2 d. The reaction mixture was concentrated and the residue was separated by means of preparative HPLC (eluent: acetonitrile/water with 0.1% TFA (gradient)). The product-containing fractions were combined and admixed with 0.5 ml of 4M hydrogen chloride in 1,4-dioxane and the mixture was concentrated on a rotary evaporator. The residue was dried under high vacuum. This gave 51 mg (71% of theory) of the title compound.

¹H NMR (400 MHz, DMSO-d₆): δ=0.85-0.99 (m, 2H), 1.12-1.36 (m, 2H), 1.43-1.63 (m, 2H), 1.70-1.83 (m, 3H), 2.11-2.21 (m, 1H), 2.22 (s, 3H), 2.58-2.69 (m, 2H), 2.92-3.03 (m, 3H), 3.14 (dd, 1H), 3.44-3.56, 3.63-3.72 (m, m, 5H), 4.70-4.77 (m, 1H), 7.21-7.28 (m, 3H), 7.40 (d, 2H), 7.77 (d, 1H), 7.80-7.92 (m, 5H), 7.96-8.10 (m, 4H), 8.31 (d, 1H), 8.74 (t, 1H), 10.59 (s, 1H).

LC-MS (Method 1): Rₜ=0.58 min; MS (ESIneg): m/z=622 [M−H—HCl]⁻.

### Example 16

### trans-4-(Aminomethyl)-N-[(2S)-3-[2′-methyl-4′-(piperazin-1-ylcarbonyl)biphenyl-4-yl]-1-oxo-1-{[4-(2H-tetrazol-5-yl)phenyl]amino}propan-2-yl]cyclohexanecarboxamide hydrochloride

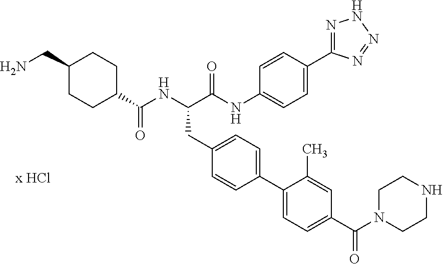

A solution of 92 mg (0.10 mmol) of tert-butyl 4-({4′-[(2S)-2-{[(trans-4-{[(tert-butoxycarbonyl)-amino]methyl}cyclohexyl)-carbonyl]amino}-3-oxo-3-{[4-(2H-tetrazol-5-yl)phenyl]amino}propyl]-2-methylbiphenyl-4-yl}carbonyl)piperazine-1-carboxylate in 3 ml of 1,4-dioxane was admixed with 0.36 ml (1.44 mmol) of 4M hydrogen chloride in 1,4-dioxane and stirred at RT overnight. The reaction mixture was separated directly by means of preparative HPLC (eluent: acetonitrile/water with 0.1% TFA (gradient)). The product-containing fractions were combined and admixed with a little 4M hydrogen chloride in 1,4-dioxane and concentrated on a rotary evaporator. The residue was dried under high vacuum. This gave 65 mg (91% of theory) of the title compound.

¹H NMR (400 MHz, DMSO-d₆): δ=0.86-1.01 (m, 2H), 1.13-1.34 (m, 2H), 1.42-1.63 (m, 2H), 1.70-1.83 (m, 3H), 2.11-2.25 (m, 1H), 2.21 (s, 3H), 2.58-2.70 (m, 2H), 2.96 (dd, 1H), 3.08-3.22 (m, 3H), 3.43-3.52 (m, 1H), 3.4-4.1 (m, 5H), 4.70-4.77 (m, 1H), 7.21-7.28 (m, 3H), 7.33 (d, 1H), 7.36-7.42 (m, 3H), 7.78-7.92 (m, 5H), 8.02 (d, 2H), 8.31 (d, 1H), 9.3 (bs, 1H), 10.58 (s, 1H).

LC-MS (Method 1): Rₜ=0.57 min; MS (ESIneg): m/z=648 [M−H—HCl]⁻.

### Example 17

### 4′-[(2S)-2-({[trans-4-(Aminomethyl)cyclohexyl]carbonyl}amino)-3-(1H-indazol-6-ylamino)-3-oxopropyl]-N-(2-hydroxyethyl)-2-methylbiphenyl-4-carboxamide hydrochloride

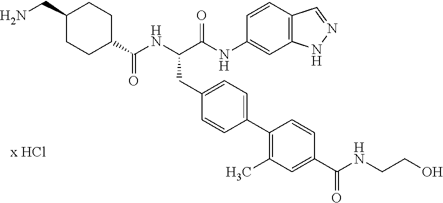

A solution of 99 mg (0.11 mmol) of tert-butyl [(trans-4-{[(2S)-3-{4′-[(2-tert-butoxyethyl)carbamoyl]-2′-methylbiphenyl-4-yl}-1-(1H-indazol-6-ylamino)-1-oxopropan-2-yl]carbamoyl}cyclohexyl)methyl]carbamate trifluoroacetate in 3 ml of 1,4-dioxane was admixed with 0.43 ml (1.71 mmol) of 4M hydrogen chloride in 1,4-dioxane and stirred at RT for 5.5 d. The precipitated solid was filtered off, washed with a little acetonitrile and dried under high vacuum. The crude product was taken up in methanol and separated by means of preparative HPLC (eluent: acetonitrile/water with 0.1% TFA (gradient)). The product-containing fractions were combined and admixed with 0.5 ml of 4M hydrogen chloride in 1,4-dioxane and the mixture was concentrated on a rotary evaporator. The residue was dried under high vacuum. This gave 43 mg (51% of theory, 90% purity) of the title compound.

¹H NMR (400 MHz, DMSO-d₆): δ=0.81-1.01 (m, 2H), 1.08-1.35 (m, 2H), 1.41-1.62 (m, 2H), 1.66-1.83 (m, 3H), 2.22 (m, 4H), 2.58-2.68 (m, 2H), 2.90-3.01 (m, 1H), 3.08-3.17 (m, 1H), 3.28-3.38 (m, 2H), 3.46-3.55 (m, 2H), 4.71-4.83 (m, 1H), 7.10-7.17 (m, 1H), 7.18-7.30 (m, 3H), 7.36-7.44 (m, 2H), 7.63-7.75 (m, 2H), 7.76-7.93 (m, 4H), 7.98 (s, 1H), 8.14 (s, 1H), 8.27 (d, 1H), 8.40-8.49 (m, 1H), 10.36 (m, 1H), 12.9 (bs, 1H).

LC-MS (Method 1): Rₜ=0.67 min; MS (ESIneg): m/z=595 [M−H—HCl]⁻.

### Example 18

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(2-hydroxyethyl)-2-methylbiphenyl-4-carboxamide hydrochloride

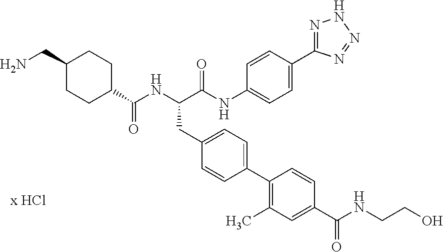

A solution of 81 mg (0.09 mmol) of tert-butyl [(trans-4-{[(2S)-3-{4′-[(2-tert-butoxyethyl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate in 3 ml of 1,4-dioxane was admixed with 0.34 ml (1.36 mmol) of 4M hydrogen chloride in 1,4-dioxane and stirred at RT for 3.5 d. The reaction mixture was concentrated; the residue was taken up in methanol and separated by means of preparative HPLC (eluent: acetonitrile/water with 0.1% TFA (gradient)). The product-containing fractions were combined and admixed with 0.5 ml of 4M hydrogen chloride in 1,4-dioxane and the mixture was concentrated on a rotary evaporator. The residue was dried under high vacuum. This gave 50 mg (71% of theory, 90% purity) of the title compound.

¹H NMR (400 MHz, DMSO-d₆): δ=0.83-1.01 (m, 2H), 1.09-1.35 (m, 2H), 1.40-1.63 (m, 2H), 1.65-1.83 (m, 3H), 2.10-2.29 (m, 4H), 2.59-2.69 (m, 2H), 2.90-3.01 (m, 1H), 3.08-3.19 (m, 1H), 3.28-3.37, 3.43-3.56, 3.64-3.74 (m, 5H), 4.70-4.81 (m, 1H), 7.19-7.32 (m, 4H), 7.40 (d, 2H), 7.68-7.90 (m, 7H), 8.02 (d, 2H), 8.28 (d, 1H), 8.40-8.48 (m, 1H), 10.57 (s, 1H).

LC-MS (Method 1): Rₜ=0.67 min; MS (ESIneg): m/z=623 [M−H—HCl]⁻.

### Example 19

### 4′-[(2S)-2-({[trans-4-(Aminomethyl)cyclohexyl]carbonyl}amino)-3-oxo-3-{[4-(2H-tetrazol-5-yl)phenyl]amino}propyl]-N,2-dimethylbiphenyl-4-carboxamide

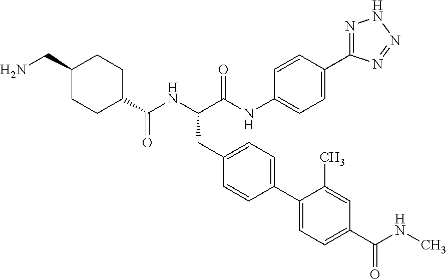

A solution of 72 mg (0.09 mmol) of tert-butyl [(trans-4-{[(2S)-3-[2′-methyl-4′-(methylcarbamoyl)biphenyl-4-yl]-1-oxo-1-{[4-(2H-tetrazol-5-yl)phenyl]amino}propan-2-yl]carbamoyl}-cyclohexyl)methyl]carbamate in 2.5 ml of 1,4-dioxane was admixed with 0.33 ml (1.34 mmol) of 4M hydrogen chloride in 1,4-dioxane and stirred at RT for 2.5 d. The reaction mixture was concentrated on a rotary evaporator; the residue was dissolved in a little water/methanol/trifluoroacetic acid and separated by means of preparative HPLC (column: Shield RP18, 5 μm, 100 mm×19 mm; eluent: water/acetonitrile/2% ammonia solution 90:5:5, 0-8.5 min; water/acetonitrile/2% ammonia solution 59:36:5, 8.5-8.6 min; water/acetonitrile/2% ammonia solution 90:5:5, 8.6-10 min; flow rate: 40 ml/min; temperature: RT). The product-containing fractions were concentrated on a rotary evaporator. After lyophilization overnight, 8 mg (12% of theory, 84% purity) of the title compound were obtained.

LC-MS (Method 1): Rₜ=0.71 min; MS (ESIneg): m/z=593 [M−H]⁻.

### Example 20

### trans-4-(Aminomethyl)-N-[(2S)-3-[2′-methyl-4′-(pyrrolidin-1-ylcarbonyl)biphenyl-4-yl]-1-oxo-1-{[4-(2H-tetrazol-5-yl)phenyl]amino}propan-2-yl]cyclohexanecarboxamide

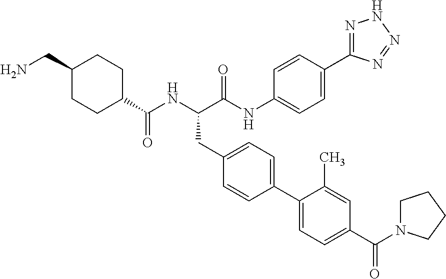

A solution of 43 mg (0.05 mmol, 50% purity) of tert-butyl [(trans-4-{[(2S)-3-[2′-methyl-4′-(pyrrolidin-1-ylcarbonyl)biphenyl-4-yl]-1-oxo-1-{[4-(2H-tetrazol-5-yl)phenyl]amino}propan-2-yl]carbamoyl}-cyclohexyl)methyl]carbamate in 2 ml of 1,4-dioxane was admixed with 0.19 ml (0.76 mmol) of 4M hydrogen chloride in 1,4-dioxane and stirred at RT overnight. The reaction mixture was concentrated on a rotary evaporator; the residue was dissolved in a little methanol/acetonitrile and separated by means of preparative HPLC (column: Shield RP18, 5 μm, 100 mm×19 mm; eluent: water/acetonitrile/2% aqueous ammonia solution 90:5:5, 0-1 min; acetonitrile/2% aqueous ammonia solution 0:95:5, 1-13.1 min; water/acetonitrile/2% aqueous ammonia solution 90:5:5, 13.1-15 min; flow rate: 40 ml/min; temperature: RT; UV detection: 210 nm). The product-containing fractions were concentrated on a rotary evaporator. After lyophilization overnight, 4 mg (12% of theory) of the title compound were obtained.

LC-MS (Method 12): Rₜ=1.71 min; MS (ESIneg): m/z=633 [M−H]⁻.

### Example 21

### 4′-[(2R,S)-2-({[trans-4-(Aminomethyl)cyclohexyl]carbonyl}amino)-3-(1H-indazol-6-ylamino)-3-oxopropyl]-N-isopropyl-2-methylbiphenyl-4-carboxamide hydrochloride (enantiomer mixture)

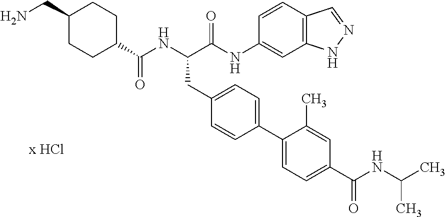

A solution of 92 mg (0.11 mmol) of tert-butyl {[trans-4-({(2S)-1-(1H-indazol-6-ylamino)-3-[4′-(isopropylcarbamoyl)-2′-methylbiphenyl-4-yl]-1-oxopropan-2-yl}carbamoyl)cyclohexyl]methyl}-carbamate trifluoroacetate in 3 ml of 1,4-dioxane was admixed with 0.43 ml (1.71 mmol) of 4M hydrogen chloride in 1,4-dioxane and stirred at RT overnight. The precipitate was filtered off, washed with a little acetonitrile and dried under high vacuum. This gave 73 mg (96% of theory) of the title compound. By analytical HPLC on a chiral column, it was found that the product was an enantiomer mixture.

¹H NMR (400 MHz, DMSO-d₆): δ=0.83-1.00 (m, 2H), 1.08-1.35 (m, 8H), 1.40-1.62 (m, 2H), 1.67-1.82 (m, 3H), 2.09-2.28 (m, 4H), 2.58-2.69 (m, 2H), 2.90-3.01 (m, 1H), 3.08-3.19 (m, 1H), 4.04-4.17 (m, 1H), 4.72-4.82 (m, 1H), 7.14 (dd, 1H), 7.18-7.31 (m, 3H), 7.40 (d, 2H), 7.63-7.73 (m, 2H), 7.73-7.89 (m, 4H), 7.98 (s, 1H), 8.14 (s, 1H), 8.21 (d, 1H), 8.26 (d, 1H), 10.34 (s, 1H), 12.9 (bs, 1H).

LC-MS (Method 1): Rₜ=0.79 min; MS (ESIneg): m/z=593 [M−H—HCl]⁻.

### Example 22

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-2-methyl-N-(piperidin-4-yl)biphenyl-4-carboxamide hydrochloride

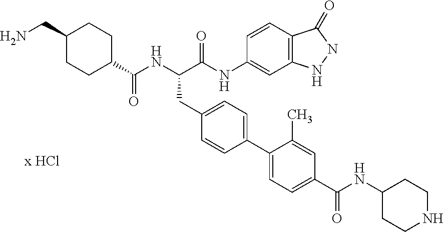

A solution of 107 mg (0.11 mmol) of tert-butyl 4-{[(4′-{(2S)-2-{[(trans-4-{[(tert-butoxycarbonyl)amino]methyl}cyclohexyl)-carbonyl]amino}-3-oxo-3-[(3-oxo-2,3-dihydro-1H-indazol-6-yl)amino]propyl}-2-methylbiphenyl-4-yl)carbonyl]amino}piperidine-1-carboxylate in 3 ml of 1,4-dioxane was admixed with 0.42 ml (1.66 mmol) of 4M hydrogen chloride in 1,4-dioxane and stirred at RT overnight. The precipitate was filtered off, washed with a little acetonitrile and dried under high vacuum. This gave 81 mg (quant.) of the title compound.

¹H NMR (400 MHz, DMSO-d₆): δ=0.82-1.00 (m, 2H), 1.09-1.34 (m, 2H), 1.39-1.62 (m, 2H), 1.65-1.85 (m, 5H), 1.90-2.02 (m, 2H), 2.09-2.20 (m, 1H), 2.24 (s, 3H), 2.59-2.68 (m, 2H), 2.86-3.13 (m, 4H), 3.25-3.35 (m, 2H), 4.01-4.13 (m, 1H), 4.63-4.74 (m, 1H), 6.83 (d, 1H), 7.03 (d, 1H), 7.20-7.30 (m, 3H), 7.38 (d, 2H), 7.44 (s, 1H), 7.67-7.90 (m, 5H), 8.21 (d, 1H), 8.49 (d, 1H), 8.65-8.87 (m, 2H), 10.04 (s, 1H), 10.52 (s, 1H), 10.58 (s, 1H).

LC-MS (Method 2): Rₜ=1.35 min; MS (ESIneg): m/z=650 [M−H—HCl]⁻.

### Example 23

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-N-[2-(diethylamino)ethyl]-2-methylbiphenyl-4-carboxamide hydrochloride

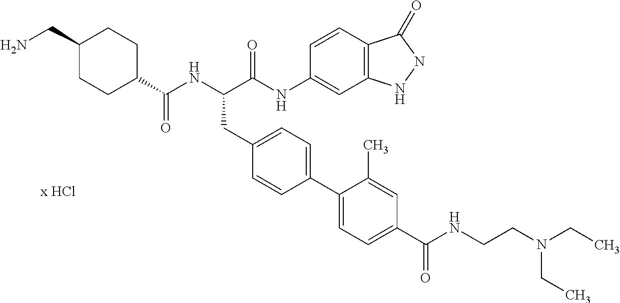

A solution of 57 mg (0.07 mmol) of tert-butyl {[trans-4-({(2S)-3-(4′-{[2-(diethylamino)ethyl]-carbamoyl}-2′-methylbiphenyl-4-yl)-1-oxo-1-[(3-oxo-2,3-dihydro-1H-indazol-6-yl)amino]propan-2-yl}carbamoyl)cyclohexyl]methyl}carbamate trifluoroacetate in 2 ml of 1,4-dioxane was admixed with 0.24 ml (0.97 mmol) of 4M hydrogen chloride in 1,4-dioxane and stirred at RT overnight. The precipitate was filtered off, washed with a little acetonitrile and dried under high vacuum. This gave 36 mg (75% of theory) of the title compound.

¹H NMR (400 MHz, DMSO-d₆): δ=0.82-0.99 (m, 2H), 1.10-1.35 (m, 8H), 1.39-1.62 (m, 2H), 1.66-1.81 (m, 3H), 2.09-2.20 (m, 1H), 2.24 (s, 3H), 2.59-2.69 (m, 2H), 2.87-2.97 (m, 1H), 3.04-3.12 (m, 1H), 3.14-3.28 (m, 6H), 3.60-3.70 (m, 2H), 4.65-4.75 (m, 1H), 6.83 (d, 1H), 6.99-7.06 (m, 1H), 7.21-7.31 (m, 3H), 7.38 (d, 2H), 7.43 (s, 1H), 7.69-7.88 (m, 5H), 8.20 (d, 1H), 8.83-8.92 (m, 1H), 9.90 (bs, 1H), 10.02 (s, 1H), 10.52 (s, 1H), 10.58 (s, 1H).

LC-MS (Method 2): Rₜ=1.38 min; MS (ESIneg): m/z=666 [M−H—HCl]⁻.

### Example 24

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-N-[3-(diethylamino)propyl]-2-methylbiphenyl-4-carboxamide hydrochloride

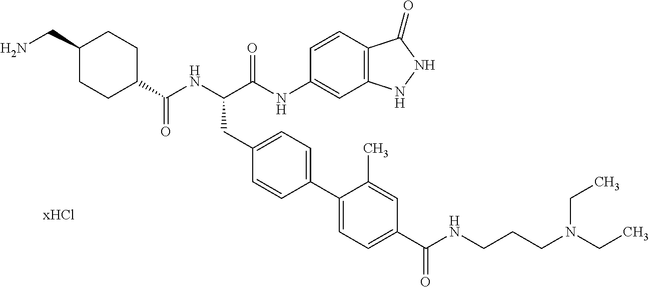

A solution of 41 mg (0.05 mmol) of tert-butyl {[trans-4-({(2S)-3-(4′-{[3-(diethylamino)propyl]-carbamoyl}-2′-methylbiphenyl-4-yl)-1-oxo-1-[(3-oxo-2,3-dihydro-1H-indazol-6-yl)amino]propan-2-yl}carbamoyl)cyclohexyl]methyl}carbamate trifluoroacetate in 2 ml of 1,4-dioxane was admixed with 0.17 ml (0.69 mmol) of 4M hydrogen chloride in 1,4-dioxane and stirred at RT overnight. The precipitate was filtered off, washed with a little acetonitrile and dried under high vacuum. This gave 26 mg (75% of theory) of the title compound.

¹H NMR (400 MHz, DMSO-d₆): δ=0.83-0.99 (m, 2H), 1.10-1.34 (m, 8H), 1.39-1.62 (m, 2H), 1.66-1.81 (m, 3H), 1.84-1.97 (m, 2H), 2.09-2.19 (m, 1H), 2.24 (s, 3H), 2.58-2.68 (m, 2H), 2.87-2.97 (m, 1H), 3.01-3.17 (m, 2H), 3.29-3.39 (m, 2H), 4.64-4.75 (m, 1H), 6.84 (d, 1H), 7.00-7.07 (m, 1H), 7.19-7.29 (m, 3H), 7.38 (d, 2H), 7.44 (s, 1H), 7.67-7.89 (m, 5H), 8.21 (d, 1H), 8.64-8.73 (m, 1H), 9.9 (bs, 1H), 10.04 (s, 1H), 10.52 (s, 1H), 10.58 (s, 1H).

LC-MS (Method 2): Rₜ=1.39 min; MS (ESIneg): m/z=680 [M−H—HCl]⁻.

### Example 25

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-2-methyl-N-[(3S)-pyrrolidin-3-yl]biphenyl-4-carboxamide hydrochloride

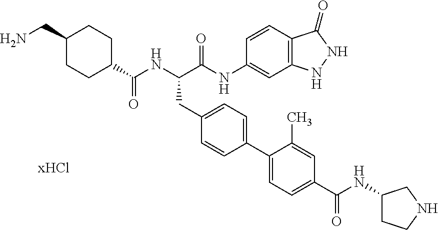

A solution of 48 mg (0.05 mmol) of tert-butyl (3S)-3-{[(4′-{(2S)-2-{[(trans-4-{[(tert-butoxycarbonyl)amino]methyl}cyclohexyl)-carbonyl]amino}-3-oxo-3-[(3-oxo-2,3-dihydro-1H-indazol-6-yl)amino]propyl}-2-methylbiphenyl-4-yl)carbonyl]amino}pyrrolidine-1-carboxylate in 2 ml of 1,4-dioxane was admixed with 0.19 ml (0.76 mmol) of 4M hydrogen chloride in 1,4-dioxane and stirred at RT overnight. The precipitate was filtered off, washed with a little acetonitrile and dried under high vacuum. This gave 39 mg (quant.) of the title compound.

¹H NMR (400 MHz, DMSO-d₆): δ=0.81-1.00 (m, 2H), 1.08-1.35 (m, 2H), 1.40-1.61 (m, 2H), 1.65-1.82 (m, 3H), 1.94-2.06 (m, 1H), 2.09-2.28 (m, 5H), 2.59-2.69 (m, 2H), 2.87-2.98 (m, 1H), 3.05-3.14 (m, 1H), 3.15-3.30 (m, 2H), 3.32-3.46 (m, 2H), 4.51-4.62 (m, 1H), 4.65-4.76 (m, 1H), 6.84 (d, 1H), 7.04 (d, 1H), 7.20-7.30 (m, 3H), 7.38 (d, 2H), 7.44 (s, 1H), 7.73-7.90 (m, 5H), 8.20 (d, 1H), 8.75 (d, 1H), 9.1 (bs, 1H), 9.3 (bs, 1H), 10.04 (s, 1H), 10.52 (s, 1H), 10.58 (s, 1H).

LC-MS (Method 2): Rₜ=1.33 min; MS (ESIneg): m/z=636 [M−H—HCl]⁻.

### Example 26

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-N-(2,5,8, 11-tetraoxatridecan-13-yl)biphenyl-4-carboxamide hydrochloride

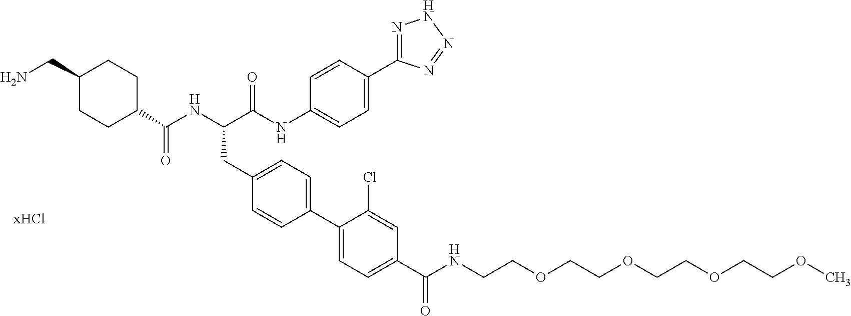

A solution of 76 mg (0.08 mmol) of tert-butyl [(trans-4-{[(2S)-3-[2′-chloro-4′-(2,5,8,11-tetraoxatridecan-13-ylcarbamoyl)biphenyl-4-yl]-1-oxo-1-{[4-(2H-tetrazol-5-yl)phenyl]amino}-propan-2-yl]carbamoyl}cyclohexyl)methyl]carbamate in 2.5 ml of 1,4-dioxane was admixed with 0.28 ml (1.13 mmol) of 4M hydrogen chloride in 1,4-dioxane and stirred at RT overnight. The precipitate was filtered off, washed with a little acetonitrile and dried under high vacuum. This gave 63 mg (93% of theory) of the title compound.

¹H NMR (400 MHz, DMSO-d₆): δ=0.83-1.01 (m, 2H), 1.11-1.35 (m, 2H), 1.40-1.64 (m, 2H), 1.67-1.83 (m, 3H), 2.09-2.20 (m, 1H), 2.59-2.70 (m, 2H), 2.90-3.02 (m, 1H), 3.09-3.19 (m, 1H), 3.22 (s, 3H), 3.26-3.61 (m, 16H), 4.70-4.81 (m, 1H), 7.33-7.51 (m, 5H), 7.67-7.92 (m, 6H), 7.97-8.07 (m, 3H), 8.29 (d, 1H), 8.68-8.77 (m, 1H), 10.56 (s, 1H).

LC-MS (Method 1): Rₜ=0.74 min; MS (ESIneg): m/z=789 [M−H—HCl]⁻.

### Example 27

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-(2,5,8,11-tetraoxatridecan-13-yl)biphenyl-4-carboxamide hydrochloride

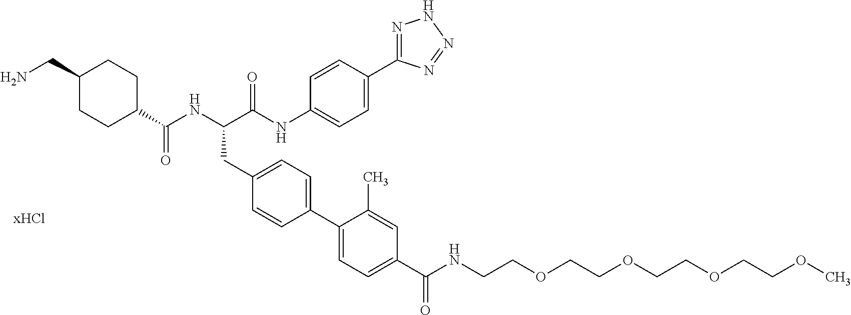

A solution of 76 mg (0.08 mmol) of tert-butyl [(trans-4-{[(2S)-3-[2′-methyl-4′-(2,5,8,11-tetraoxatridecan-13-ylcarbamoyl)biphenyl-4-yl]-1-oxo-1-{[4-(2H-tetrazol-5-yl)phenyl]amino}-propan-2-yl]carbamoyl}cyclohexyl)methyl]carbamate in 2.5 ml of 1,4-dioxane was admixed with 0.29 ml (1.16 mmol) of 4M hydrogen chloride in 1,4-dioxane and stirred at RT overnight. The precipitate was filtered off and washed with a little acetonitrile, dried under high vacuum and then separated by means of preparative HPLC (eluent: acetonitrile/water with 0.1% TFA (gradient)). The product-containing fractions were combined and admixed with 0.1 ml of 4M hydrogen chloride in 1,4-dioxane and the mixture was concentrated on a rotary evaporator. The residue was dried under high vacuum. This gave 40 mg (57% of theory, 93% purity) of the title compound.

¹H NMR (400 MHz, DMSO-d₆): δ=0.83-1.01 (m, 2H), 1.09-1.34 (m, 2H), 1.40-1.62 (m, 2H), 1.66-1.82 (m, 3H), 2.10-2.25 (m, 4H), 2.58-2.69 (m, 2H), 2.91-3.01 (m, 1H), 3.08-3.18 (m, 1H), 3.22 (s, 3H), 3.36-3.59 (m, 16H), 4.70-4.80 (m, 1H), 7.19-7.31 (m, 3H), 7.40 (d, J=8.07 Hz, 2H), 7.67-7.89 (m, 7H), 8.02 (d, 2H), 8.28 (d, 1H), 8.47-8.55 (m, 1H), 10.55 (s, 1H).

LC-MS (Method 1): Rₜ=0.74 min; MS (ESIneg): m/z=769 [M−H—HCl]⁻.

### Example 28

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-2-methyl-N-(2,5,8,11-tetraoxatridecan-13-yl)biphenyl-4-carboxamide hydrochloride

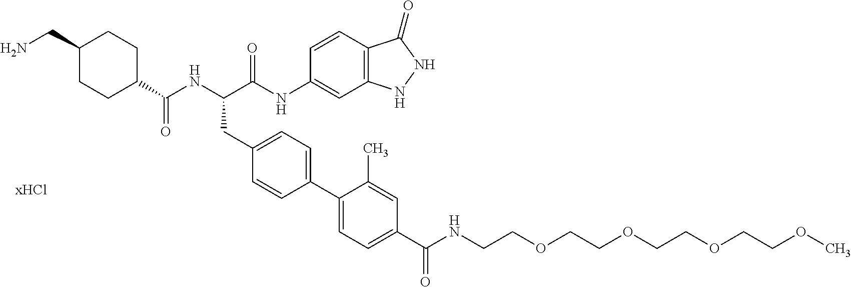

A solution of 60 mg (0.06 mmol) of tert-butyl {[trans-4-({(2S)-3-[2′-methyl-4′-(2,5,8,11-tetraoxatridecan-13-ylcarbamoyl)biphenyl-4-yl]-1-oxo-1-[(3-oxo-2,3-dihydro-1H-indazol-6-yl)amino]propan-2-yl}carbamoyl)cyclohexyl]methyl}carbamate in 2 ml of 1,4-dioxane was admixed with 0.23 ml (0.93 mmol) of 4M hydrogen chloride in 1,4-dioxane and stirred at RT overnight. The precipitate was filtered off, washed with a little acetonitrile and dried under high vacuum. This gave 46 mg (86% of theory) of the title compound.

¹H NMR (400 MHz, DMSO-d₆): δ=0.82-0.99 (m, 2H), 1.08-1.34 (m, 2H), 1.38-1.62 (m, 2H), 1.63-1.83 (m, 3H), 2.06-2.19 (m, 1H), 2.23 (s, 3H), 2.59-2.69 (m, 2H), 2.87-2.97 (m, 1H), 3.04-3.14 (m, 1H), 3.22 (s, 3H), 3.29-3.62 (m, 16H), 4.65-4.75 (m, 1H), 6.84 (d, 1H), 6.99-7.06 (m, 1H), 7.20-7.31 (m, 3H), 7.34-7.47 (m, 3H), 7.66-7.84 (m, 5H), 8.18 (d, 1H), 8.47-8.55 (m, 1H), 10.02 (s, 1H), 10.52 (s, 1H), 10.58 (s, 1H).

LC-MS (Method 1): Rₜ=0.68 min; MS (ESIneg): m/z=757 [M−H—HCl]⁻.

### Example 29

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[3-(diethylamino)propyl]-2-methylbiphenyl-4-carboxamide hydrochloride

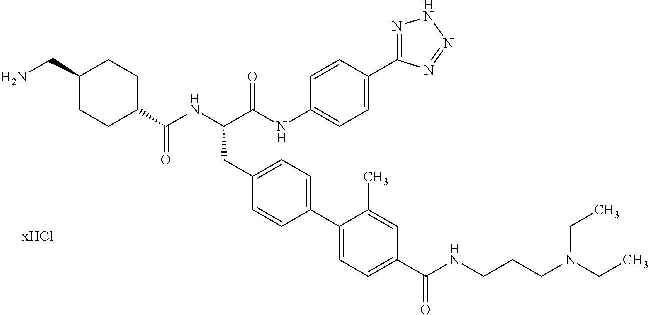

A solution of 80 mg (0.09 mmol) of tert-butyl [(trans-4-{[(2S)-3-(4′-{[3-(diethylamino)-propyl]carbamoyl}-2′-methylbiphenyl-4-yl)-1-oxo-1-{[4-(2H-tetrazol-5-yl)phenyl]amino}propan-2-yl]carbamoyl}cyclohexyl]methyl}carbamate trifluoroacetate in 3 ml of 1,4-dioxane was admixed with 0.33 ml (1.32 mmol) of 4M hydrogen chloride in 1,4-dioxane and stirred at RT overnight. The precipitate was filtered off, washed with a little acetonitrile and dried under high vacuum. This gave 56 mg (81% of theory) of the title compound.

¹H NMR (400 MHz, DMSO-d₆): δ=0.84-1.00 (m, 2H), 1.11-1.34 (m, 8H), 1.41-1.63 (m, 2H), 1.68-1.82 (m, 3H), 1.85-1.97 (m, 2H), 2.11-2.26 (m, 4H), 2.58-2.69 (m, 2H), 2.91-3.01 (m, 1H), 3.03-3.19 (m, 7H), 3.29-3.40 (m, 2H), 4.70-4.80 (m, 1H), 7.21-7.32 (m, 3H), 7.40 (d, 2H), 7.69-7.91 (m, 7H), 8.02 (d, 2H), 8.31 (d, 1H), 8.64-8.73 (m, 1H), 9.9 (br. s, 1H), 10.56 (s, 1H).

LC-MS (Method 1): Rₜ=0.64 min; MS (ESIneg): m/z=692 [M−H—HCl]⁻.

### Example 30

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-N-(2-methylpiperidin-4-yl)biphenyl-4-carboxamide hydrochloride

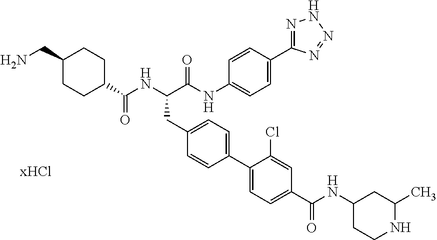

A solution of 67 mg (0.07 mmol) of tert-butyl 4-({4′-[(2S)-2-{[(trans-4-{[(tert-butoxycarbonyl)-amino]methyl}cyclohexyl)-carbonyl]amino}-3-oxo-3-({[4-(2H-tetrazol-5-yl)phenyl]amino}propyl]-2-chlorobiphenyl-4-yl}carbonyl)amino]-2-methylpiperidine-1-carboxylate in 2.5 ml of 1,4-dioxane was admixed with 0.25 ml (0.99 mmol) of 4M hydrogen chloride in 1,4-dioxane and stirred at RT overnight. The reaction mixture was concentrated and separated directly by means of preparative HPLC (eluent: acetonitrile/water with 0.1% TFA (gradient)). The product-containing fractions were combined and admixed with 0.1 ml of 4M hydrogen chloride in 1,4-dioxane and the mixture was concentrated on a rotary evaporator. The residue was dried under high vacuum. This gave 43 mg (81% of theory) of the title compound.

¹H NMR (400 MHz, DMSO-d₆): δ=0.84-1.02 (m, 2H), 1.08-1.36 (m, 5H), 1.40-1.65 (m, 3H), 1.65-1.84 (m, 4H), 1.88-2.07 (m, 2H), 2.09-2.21 (m, 1H), 2.59-2.68 (m, 2H), 2.89-3.08 (m, 2H), 3.10-3.21 (m, 2H), 3.22-3.36 (m, 2H), 4.69-4.82 (m, 1H), 7.29-7.51 (m, 5H), 7.72-7.95 (m, 6H), 8.03 (d, 3H), 8.25-8.37 (m, 1H), 8.65-8.75 (m, 1H), 8.80-8.96 (m, 1H), 9.00-9.14 (m, 1H), 10.61 (s, 1H)

LC-MS (Method 1): Rₜ=0.63 min; MS (ESIneg): m/z=696 [M−H—HCl]⁻.

### Example 31

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-2-methyl-N-(2-methylpiperidin-4-yl)biphenyl-4-carboxamide hydrochloride

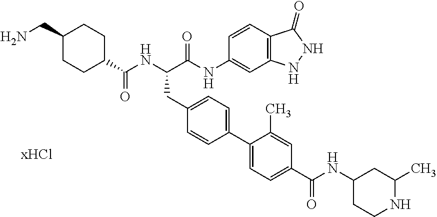

A solution of 48 mg (0.05 mmol) of tert-butyl 4-{[(4′-{(2S)-2-{[(trans-4-{[(tert-butoxycarbonyl)amino]methyl}cyclohexyl)-carbonyl]amino}-3-oxo-3-[(3-oxo-2,3-dihydro-1H-indazol-6-yl)amino]propyl}-2-methylbiphenyl-4-yl)carbonyl]amino}-2-methylpiperidine-1-carboxylate in 2 ml of 1,4-dioxane was admixed with 0.18 ml (0.74 mmol) of 4M hydrogen chloride in 1,4-dioxane and stirred at RT overnight. The precipitate was filtered off, washed with a little acetonitrile and dried under high vacuum. This gave 38 mg (quant.) of the title compound.

¹H NMR (400 MHz, DMSO-d₆): δ=0.82-1.00 (m, 2H), 1.09-1.34 (m, 5H), 1.39-1.62 (m, 3H), 1.64-1.82 (m, 4H), 1.85-2.06 (m, 2H), 2.09-2.19 (m, 1H), 2.23 (s, 3H), 2.59-2.69 (m, 2H), 2.85-3.13 (m, 3H), 3.17-3.49 (m, 3H), 4.02-4.15 (m, 1H), 4.65-4.75 (m, 1H), 6.83 (d, 1H), 7.00-7.07 (m, 1H), 7.19-7.29 (m, 3H), 7.38 (d, 2H), 7.44 (s, 1H), 7.69-7.93 (m, 5H), 8.20 (d, 1H), 8.51 (d, 1H), 8.68-8.83 (m, 1H), 8.94-9.06 (m, 1H), 10.03 (s, 1H), 10.52 (s, 1H), 10.58 (s, 1H).

LC-MS (Method 1): Rₜ=0.59 min; MS (ESIneg): m/z=664 [M−H—HCl]⁻.

### Example 32

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-(2-methylpiperidin-4-yl)biphenyl-4-carboxamide hydrochloride

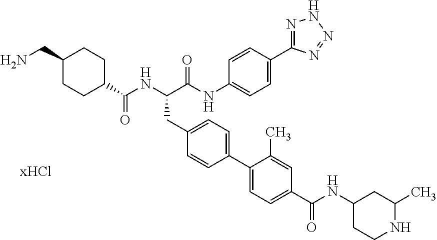

A solution of 53 mg (0.05 mmol) of tert-butyl 4-({4′-[(2S)-2-{[(trans-4-{[(tert-butoxycarbonyl)-amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]-2-methylpiperidine-1-carboxylate in 2 ml of 1,4-dioxane was admixed with 0.20 ml (0.80 mmol) of 4M hydrogen chloride in 1,4-dioxane and stirred at RT overnight. The reaction mixture was concentrated and separated directly by means of preparative HPLC (eluent: acetonitrile/water with 0.1% TFA (gradient)). The product-containing fractions were combined and admixed with 0.1 ml of 4M hydrogen chloride in 1,4-dioxane and the mixture was concentrated on a rotary evaporator. The residue was dried under high vacuum. This gave 32 mg (78% of theory) of the title compound.

¹H NMR (400 MHz, DMSO-d₆): δ=0.84-1.00 (m, 2H), 1.11-1.34 (m, 5H), 1.39-1.63 (m, 3H), 1.66-1.83 (m, 4H), 1.86-2.08 (m, 3H), 2.10-2.29 (m, 1H), 2.89-3.19 (m, 3H), 3.22-3.35 (m, 2H), 4.01-4.15 (m, 1H), 4.70-4.80 (m, 1H), 7.19-7.31 (m, 3H), 7.36-7.44 (m, 2H), 7.68-7.92 (m, 6H), 8.02 (d, 2H), 8.30 (d, 1H), 8.50 (d, 1H), 8.67-8.81 (m, 1H), 8.92-9.04 (m, 1H), 10.55 (s, 1H).

LC-MS (Method 1): Rₜ=0.58 min; MS (ESIneg): m/z=676 [M−H—HCl]⁻.

### Example 33

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(trans-4-hydroxycyclohexyl)-2-methylbiphenyl-4-carboxamide hydrochloride

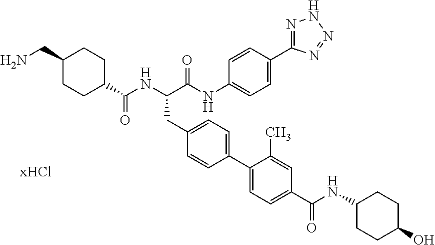

A solution of 38 mg (0.04 mmol) of tert-butyl [(trans-4-{[(2S)-3-{4′-[(trans-4-hydroxycyclohexyl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate in 1.5 ml of 1,4-dioxane was admixed with 0.16 ml (0.64 mmol) of 4M hydrogen chloride in 1,4-dioxane and stirred at RT for 2.5 d. The reaction mixture was concentrated and separated directly by means of preparative HPLC (eluent: acetonitrile/water with 0.1% TFA (gradient)). The product-containing fractions were combined and admixed with 0.1 ml of 4M hydrogen chloride in 1,4-dioxane and the mixture was concentrated on a rotary evaporator. The residue was dried under high vacuum. This gave 24 mg (71% of theory) of the title compound.

¹H NMR (400 MHz, DMSO-d₆): δ=0.83-0.99 (m, 2H), 1.09-1.62 (m, 8H), 1.66-1.90 (m, 7H), 2.10-2.26 (m, 4H), 2.58-2.68 (m, 2H), 2.91-3.00 (m, 1H), 3.09-3.18 (m, 1H), 3.34-3.44 (m, 1H), 3.64-3.79 (m, 2H), 4.70-4.80 (m, 1H), 7.16-7.29 (m, 3H), 7.39 (d, 2H), 7.69 (d, 1H), 7.72-7.89 (m, 6H), 8.02 (d, 2H), 8.17 (d, 1H), 8.29 (d, 1H), 10.56 (s, 1H).

LC-MS (Method 1): Rₜ=0.65 min; MS (ESIneg): m/z=677 [M−H—HCl]⁻.

### Example 34

### N-(trans-4-Aminocyclohexyl)-4′-[(2S)-2-({[trans-4-(aminomethyl)cyclohexyl]carbonyl}amino)-3-oxo-3-{[4-(2H-tetrazol-5-yl)phenyl]amino}propyl]-2-methylbiphenyl-4-carboxamide hydrochloride

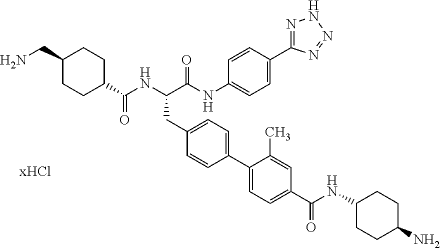

A solution of 48 mg (0.05 mmol) of tert-butyl [(trans-4-{[(2S)-3-[4′-({trans-4-{[(tert-butoxycarbonyl)amino]cyclohexyl}carbamoyl)-2′-methylbiphenyl-4-yl]-1-oxo-1-{[4-(2H-tetrazol-5-yl)phenyl]amino}propan-2-yl]carbamoyl}cyclohexyl)methyl]carbamate in 1.5 ml of 1,4-dioxane was admixed with 0.18 ml (0.73 mmol) of 4M hydrogen chloride in 1,4-dioxane and stirred at RT for 5.5 d. The reaction mixture was concentrated and separated directly by means of preparative HPLC (eluent: acetonitrile/water with 0.1% TFA (gradient)). The product-containing fractions were combined and admixed with 0.1 ml of 4M hydrogen chloride in 1,4-dioxane and the mixture was concentrated on a rotary evaporator. The residue was dried under high vacuum. This gave 19 mg (50% of theory) of the title compound.

¹H NMR (400 MHz, DMSO-d₆): δ=0.82-1.03 (m, 2H), 1.11-1.52 (m, 7H), 1.53-1.64 (m, 1H), 1.66-1.82 (m, 3H), 1.84-2.04 (m, 4H), 2.09-2.28 (m, 4H), 2.61-2.70 (m, 2H), 2.84-3.19 (m, 4H), 4.68-4.80 (m, 1H), 7.17-7.31 (m, 3H), 7.33-7.43 (m, 2H), 7.57-7.93 (m, 10H), 7.95-8.05 (m, 2H), 8.20-8.33 (m, 2H), 10.5 (br. s., 1H).

LC-MS (Method 1): Rₜ=0.55 min; MS (ESIneg): m/z=676 [M−H—HCl]⁻.

### Example 35

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[3-(dimethylamino)propyl]-2-methylbiphenyl-4-carboxamide hydrochloride

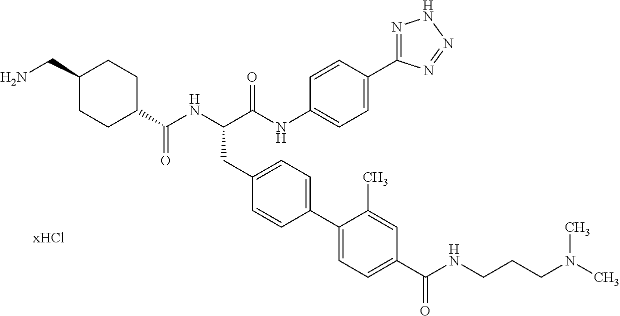

A solution of 82 mg (0.09 mmol) of tert-butyl [(trans-4-{[(2S)-3-(4′-{[3-(dimethylamino)propyl]-carbamoyl}-2′-methylbiphenyl-4-yl)-1-oxo-1-{[4-(2H-tetrazol-5-yl)phenyl]amino}propan-2-yl]carbamoyl}cyclohexyl]methyl}carbamate trifluoroacetate in 3 ml of 1,4-dioxane was admixed with 0.35 ml (1.40 mmol) of 4M hydrogen chloride in 1,4-dioxane and stirred at RT overnight. The precipitate was filtered off, washed with a little acetonitrile and dried under high vacuum. This gave 54 mg (78% of theory) of the title compound.

¹H NMR (400 MHz, DMSO-d₆): δ=0.84-1.01 (m, 2H), 1.11-1.35 (m, 2H), 1.41-1.63 (m, 2H), 1.68-1.83 (m, 3H), 1.85-1.97 (m, 2H), 2.10-2.27 (m, 4H), 2.59-2.69 (m, 2H), 2.76 (d, 6H), 2.90-3.01 (m, 1H), 3.03-3.18 (m, 3H), 3.22-3.44 (m, 2H), 4.69-4.81 (m, 1H), 7.20-7.30 (m, 3H), 7.40 (d, 2H), 7.68-7.91 (m, 7H), 8.02 (d, 2H), 8.31 (d, 1H), 8.64-8.72 (m, 1H), 10.0 (br. s, 1H), 10.56 (s, 1H), 16.8 (br. s, 1H)

LC-MS (Method 1): Rₜ=0.56 min; MS (ESIneg): m/z=664 [M−H—HCl]⁻.

### Example 36

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-(piperidin-4-yl)biphenyl-4-carboxamide

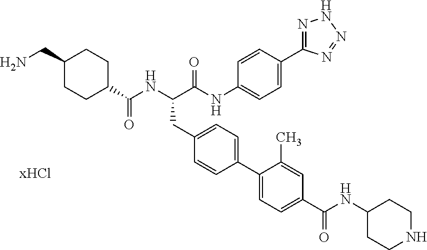

1.00 g of 4′-[(2S)-2-({[trans-4-(aminomethyl)cyclohexyl]carbonyl}amino)-3-oxo-3-{[4-(2H-tetrazol-5-yl)phenyl]amino}propyl]-2-methyl-N-(piperidin-4-yl)biphenyl-4-carboxamide hydrochloride was dissolved in 30 ml of methanol and filtered in portions through Varian Mega Bond Elut PSA (10 g) cartridges. The filtrate was concentrated on a rotary evaporator and the residue was dried under high vacuum. This gave 794 mg (87% of theory) of the title compound

¹H NMR (400 MHz, DMSO-d₆): δ=0.80-0.97 (m, 2H), 1.10-1.44 (m, 3H), 1.45-1.62 (m, 3H), 1.66-1.84 (m, 5H), 2.09-2.20 (m, 1H), 2.23 (s, 3H), 2.59 (d, 2H), 2.62-2.72 (m, 2H), 2.87-2.99 (m, 1H), 3.01-3.15 (m, 3H), 3.83-3.96 (m, 1H), 4.69-4.80 (m, 1H), 7.19-7.29 (m, 3H), 7.39 (d, 2H), 7.59 (d, 2H), 7.70 (d, 1H), 7.76 (s, 1H), 7.89 (d, 2H), 8.19 (d, 1H), 8.30 (d, 1H), 10.12 (s, 1H).

LC-MS (Method 13): Rₜ=1.30 min; MS (ESIneg): m/z=663 [M−H]⁻.

### Example 37

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(piperidin-4-yl)biphenyl-4-carboxamide hydrochloride

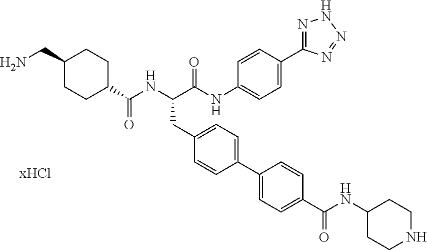

A solution of 71 mg (0.07 mmol) of tert-butyl 4-({4′-[(2S)-2-{[(trans-4-{[(tert-butoxycarbonyl)-amino]methyl}cyclohexyl)-carbonyl]amino}-3-oxo-3-{[4-(2H-tetrazol-5-yl)phenyl]amino}propyl]-biphenyl-4-yl}carbonyl)amino]piperidine-1-carboxylate in 4 ml of 1,4-dioxane was admixed with 0.28 ml (1.10 mmol) of 4M hydrogen chloride in 1,4-dioxane and stirred at RT overnight. The precipitate was filtered off, washed with a little acetonitrile and dried under high vacuum. This gave 49 mg (90% of theory) of the title compound.

¹H NMR (400 MHz, DMSO-d₆): δ=0.83-1.01 (m, 2H), 1.12-1.34 (m, 2H), 1.41-1.54 (m, 1H), 1.56-1.64 (m, 1H), 1.69-1.86 (m, 5H), 1.92-2.03 (m, 2H), 2.10-2.21 (m, 1H), 2.58-2.68 (m, 2H), 2.90-3.08 (m, 3H), 3.09-3.17 (m, 1H), 3.25-3.36 (m, 3H), 4.02-4.14 (m, 1H), 4.67-4.77 (m, 1H), 7.44 (d, 2H), 7.67 (d, 2H), 7.75 (d, 2H), 7.79-7.91 (m, 5H), 7.95 (d, 2H), 8.03 (d, 2H), 8.30 (d, 1H), 8.56 (d, 1H), 8.71-8.91 (m, 2H), 10.62 (s, 1H).

LC-MS (Method 1): Rₜ=0.59 min; MS (ESIneg): m/z=648 [M−H—HCl]⁻.

### Example 38

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-{2-[diethylamino]ethyl}biphenyl-4-carboxamide hydrochloride

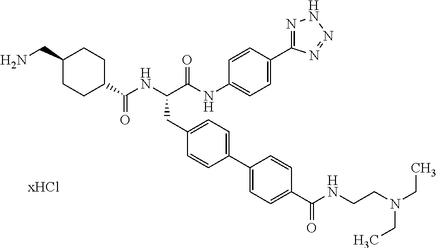

A solution of 72 mg (0.08 mmol) of tert-butyl [(trans-4-{[(2S)-3-[4′-{(2-[diethylamino]ethyl}-carbamoyl)biphenyl-4-yl]-1-oxo-1-{[4-(2H-tetrazol-5-yl)phenyl]amino}-propan-2-yl]carbamoyl}-cyclohexyl)methyl]carbamate trifluoroacetate in 4 ml of 1,4-dioxane was admixed with 0.31 ml (1.23 mmol) of 4M hydrogen chloride in 1,4-dioxane and stirred at RT overnight. The precipitate was filtered off, washed with a little acetonitrile and dried under high vacuum. This gave 46 mg (77% of theory) of the title compound.

¹H NMR (400 MHz, DMSO-d₆): δ=0.84-1.00 (m, 2H), 1.12-1.34 (m, 8H), 1.40-1.53 (m, 1H), 1.56-1.66 (m, 1H), 1.69-1.82 (m, 3H), 2.10-2.21 (m, 1H), 2.58-2.69 (m, 2H), 2.89-3.01 (m, 1H), 3.07-3.29 (m, 7H), 3.61-3.71 (m, 2H), 4.67-4.78 (m, 1H), 7.44 (d, 2H), 7.68 (d, 2H), 7.74-7.90 (m, 7H), 7.95-8.07 (m, 4H), 8.30 (d, 1H), 8.91-9.00 (m, 1H), 10.1 (br. s, 1H), 10.60 (s, 1H).

LC-MS (Method 1): Rₜ=0.63 min; MS (ESIneg): m/z=664 [M−H—HCl]⁻.

### Example 39

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[(3R)-pyrrolidin-3-yl]biphenyl-4-carboxamide hydrochloride

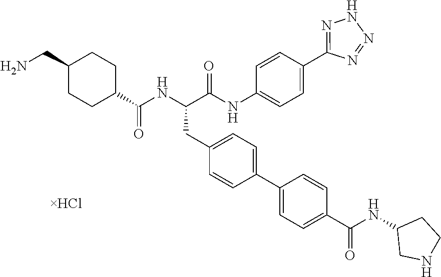

A solution of 81 mg (0.09 mmol) of tert-butyl (3R)-3-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]biphenyl-4-yl}carbonyl)amino]pyrrolidine-1-carboxylate in 4 ml of 1,4-dioxane was admixed with 0.32 ml (1.29 mmol) of 4M hydrogen chloride in 1,4-dioxane and stirred at RT overnight. The precipitate was filtered off, washed with a little acetonitrile and dried under high vacuum. This gave 57 mg (91% of theory) of the title compound.

¹H NMR (400 MHz, DMSO-d₆): δ=0.84-1.00 (m, 2H), 1.12-1.34 (m, 2H), 1.39-1.54 (m, 1H), 1.56-1.65 (m, 1H), 1.69-1.83 (m, 3H), 1.97-2.08 (m, 1H), 2.10-2.26 (m, 2H), 2.58-2.69 (m, 2H), 2.90-3.01 (m, 1H), 3.08-3.17 (m, 1H), 3.17-3.31 (m, 2H), 3.32-3.50 (m, 2H), 4.52-4.63 (m, 1H), 4.68-4.78 (m, 1H), 7.44 (d, 2H), 7.68 (d, 2H), 7.73-7.91 (m, 7H), 7.95-8.07 (m, 4H), 8.29 (dl H), 8.80 (d, 1H), 9.1 (br. s, 1H), 9.3 (br. s, 1H), 10.61 (s, 1H).

LC-MS (Method 1): Rₜ=0.54 min; MS (ESIneg): m/z=634 [M−H—HCl]⁻.

### Example 40

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(1-isopropylpiperidin-4-yl)-2-methylbiphenyl-4-carboxamide hydrochloride

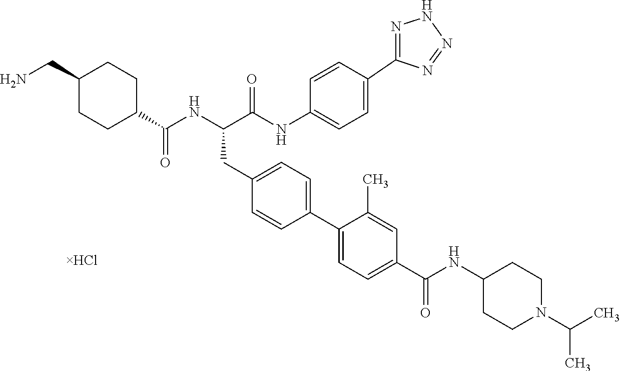

A solution of 40 mg (0.04 mmol) of tert-butyl [(trans-4-{[(2S)-3-{4′-[(1-isopropylpiperidin-4-yl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate trifluoroacetate in 1.5 ml of 1,4-dioxane was admixed with 0.16 ml (0.65 mmol) of 4M hydrogen chloride in 1,4-dioxane and stirred at RT for 2.5 d. The precipitate was filtered off, washed with a little acetonitrile and dried under high vacuum. This gave 25 mg (71% of theory) of the title compound.

¹H NMR (400 MHz, DMSO-d₆): δ=0.85-1.00 (m, 2H), 1.09-1.35 (m, 8H), 1.40-1.63 (m, 2H), 1.67-1.83 (m, 3H), 1.88-2.09 (m, 4H), 2.10-2.25 (m, 4H), 2.59-2.68 (m, 2H), 2.90-3.01 (m, 1H), 3.02-3.18 (m, 3H), 3.22-3.55 (m, 3H), 4.01-4.13 (m, 1H), 4.70-4.81 (m, 1H), 7.20-7.31 (m, 3H), 7.40 (d, 2H), 7.68-7.89 (m, 7H), 8.02 (d, 2H), 8.30 (d, 1H), 8.55 (d, 1H), 10.0 (br. s, 1H), 10.55 (s, 1H)

LC-MS (Method 1): Rₜ=0.65 min; MS (ESIneg): m/z=704 [M−H—HCl]⁻.

### Example 41

### 2-[({4′-[(2S)-2-({[trans-4-(Aminomethyl)cyclohexyl]carbonyl}amino)-3-oxo-3-({[4-(2H-tetrazol-5-yl)phenyl]amino}propyl]-2-methylbiphenyl-4-yl}carbonyl)amino]-N,N,N-trimethylethanaminium hydrochloride

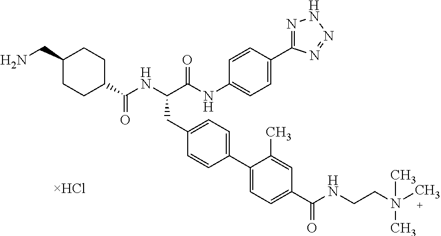

A solution of 28 mg (0.03 mmol) of 2-[({4′-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]-2-methylbiphenyl-4-yl}carbonyl)amino]-N,N,N-trimethylethanaminium trifluoroacetate in 1 ml of 1,4-dioxane was admixed with 0.12 ml (0.48 mmol) of 4M hydrogen chloride in 1,4-dioxane and stirred at RT for 2.5 d. The precipitate was filtered off, washed with a little acetonitrile and dried under high vacuum. This gave 15 mg (61% of theory) of the title compound.

¹H NMR (400 MHz, DMSO-d₆): δ=0.84-1.01 (m, 2H), 1.11-1.35 (m, 2H), 1.41-1.64 (m, 2H), 1.68-1.85 (m, 3H), 2.11-2.28 (m, 4H), 2.59-2.68 (m, 2H), 2.92-3.02 (m, 1H), 3.08-3.24 (m, 10H), 3.50-3.55 (m, 2H), 3.66-3.76 (m, 2H), 4.71-4.80 (m, 1H), 7.22-7.32 (m, 3H), 7.41 (d, 2H), 7.70-7.95 (m, 7H), 8.02 (d, 2H), 8.32 (d, 1H), 8.95-9.04 (m, 1H), 10.57 (s, 1H).

LC-MS (Method 1): Rₜ=0.61 min; MS (ESIneg): m/z=664 [M−H—HCl]⁻.

### Example 42

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-(1-methylpiperidin-4-yl)biphenyl-4-carboxamide hydrochloride

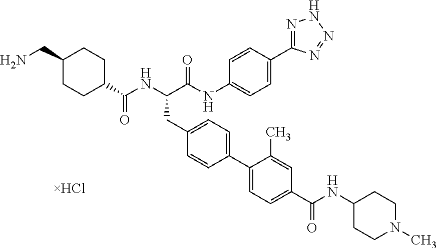

A solution of 24 mg (0.03 mmol) of tert-butyl [(trans-4-{[(2S)-3-{2′-methyl-4′-[(1-methylpiperidin-4-yl)carbamoyl]biphenyl-4-yl}-1-oxo-1-{[4-(2H-tetrazol-5-yl)phenyl]amino}-propan-2-yl]carbamoyl}cyclohexyl)methyl]carbamate trifluoroacetate in 1.5 ml of 1,4-dioxane was admixed with 0.10 ml (0.40 mmol) of 4M hydrogen chloride in 1,4-dioxane and stirred at RT overnight. The precipitate was filtered off, washed with a little acetonitrile and dried under high vacuum. This gave 17 mg (79% of theory, 94% purity) of the title compound.

¹H NMR (400 MHz, DMSO-d₆): δ=0.84-1.01 (m, 2H), 1.10-1.35 (m, 2H), 1.41-1.63 (m, 2H), 1.67-1.81 (m, 3H), 1.82-2.04 (m, 4H), 2.10-2.26 (m, 4H), 2.58-2.68 (m, 2H), 2.69-2.79 (m, 3H), 2.90-3.01 (m, 1H), 3.02-3.18 (m, 2H), 3.24-3.48 (m, 2H), 3.95-4.09 (m, 1H), 4.71-4.80 (m, 1H), 7.19-7.31 (m, 3H), 7.40 (d, 2H), 7.69-7.89 (m, 7H), 8.02 (d, 2H), 8.30 (d, 1H), 8.53 (d, 1H), 10.09-10.30 (m, 1H), 10.55 (s, 1H), 16.8 (br. s, 1H).

LC-MS (Method 1): Rₜ=0.53 min; MS (ESIneg): m/z=676 [M−H—HCl]⁻.

### Example 43

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(3-azabicyclo[3.1.0]hex-6-yl)-2-methylbiphenyl-4-carboxamide hydrochloride

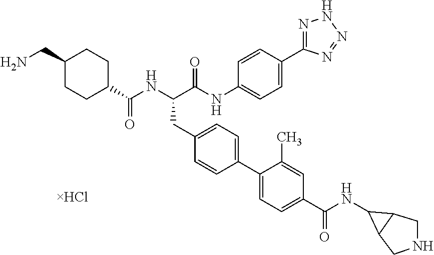

A solution of 62 mg (0.06 mmol) of tert-butyl 6-({4′-[(2S)-2-{[(trans-4-{[(tert-butoxycarbonyl)-amino]methyl}cyclohexyl)-carbonyl]amino}-3-oxo-3-{[4-(2H-tetrazol-5-yl)phenyl]amino}propyl]-2-methylbiphenyl-4-yl}carbonyl)amino]-3-azabicyclo[3.1.0]hexane-3-carboxylate in 2 ml of 1,4-dioxane was admixed with 0.24 ml (0.95 mmol) of 4M hydrogen chloride in 1,4-dioxane and stirred at RT for 2.5 d. The reaction mixture was concentrated and separated directly by means of preparative HPLC (eluent: acetonitrile/water with 0.1% TFA (gradient)). The product-containing fractions were combined and admixed with 0.1 ml of 4M hydrogen chloride in 1,4-dioxane and the mixture was concentrated on a rotary evaporator. The residue was dried under high vacuum. This gave 35 mg (73% of theory) of the title compound.

¹H NMR (400 MHz, DMSO-d₆): δ=0.83-1.00 (m, 2H), 1.10-1.34 (m, 2H), 1.41-1.62 (m, 2H), 1.67-1.83 (m, 3H), 2.0 (br. s., 2H), 2.10-2.27 (m, 4H), 2.59-2.67 (m, 2H), 2.90-3.07 (m, 2H), 3.09-3.19 (m, 1H), 3.27-3.44 (m, 4H), 4.70-4.80 (m, 1H), 7.19-7.29 (m, 3H), 7.40 (d, 2H), 7.69 (d, 1H), 7.74 (s, 1H), 7.78-7.97 (m, 5H), 8.03 (d, 2H), 8.31 (d, 1H), 8.61 (d, J=1.00 Hz, 1H), 9.3 (br. s, 1H), 9.6 (br. s, 1H), 10.59 (s, 1H).

LC-MS (Method 1): Rₜ=0.61 min; MS (ESIneg): m/z=660 [M−H—HCl]⁻.

### Example 44

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-(8-methyl-8-azabicyclo[3.2.1]oct-3-yl)biphenyl-4-carboxamide hydrochloride

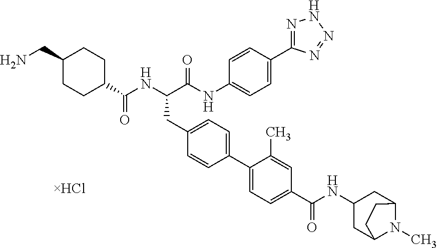

A solution of 50 mg (0.05 mmol) of tert-butyl [(trans-4-{[(2S)-3-{2′-methyl-4′-[(8-methyl-8-azabicyclo[3.2.1]oct-3-yl)carbamoyl]biphenyl-4-yl)}-1-oxo-1-[4-(2H-tetrazol-5-yl)phenyl]amino}-propan-2-yl]carbamoyl}cyclohexyl)methyl]carbamate trifluoroacetate in 2 ml of 1,4-dioxane was admixed with 0.20 ml (0.82 mmol) of 4M hydrogen chloride in 1,4-dioxane and stirred at RT overnight. The precipitate was filtered off, washed with a little acetonitrile and dried under high vacuum. This gave 38 mg (90% of theory) of the title compound.

¹H NMR (400 MHz, DMSO-d₆): δ=0.84-1.01 (m, 2H), 1.11-1.35 (m, 2H), 1.40-1.64 (m, 2H), 1.68-1.84 (m, 3H), 2.10-2.45 (m, 12H), 2.59-2.70 (m, 5H), 2.91-3.02 (m, 1H), 3.09-3.18 (m, 1H), 3.80-3.88 (m, 2H), 3.90-3.97 (m, 1H), 4.69-4.81 (m, 1H), 7.20-7.30 (m, 3H), 7.40 (d, 2H), 7.62-7.73 (m, 2H), 7.75-7.92 (m, 5H), 8.02 (d, 2H), 8.24-8.35 (m, 2H), 10.0 (br. s, 1H), 10.55 (s, 1H).

LC-MS (Method 1): Rₜ=0.55 min; MS (ESIneg): m/z=702 [M−H—HCl]⁻.

### Example 45

### 4′-[(2S)-2-({[trans-4-(Aminomethyl)cyclohexyl]carbonyl}amino)-3-oxo-3-{[4-(2H-tetrazol-5-yl)phenyl]amino}propyl]-3-fluoro-N-(piperidin-4-yl)biphenyl-4-carboxamide hydrochloride

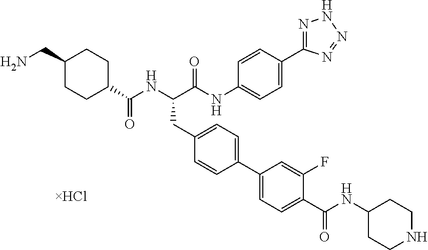

A solution of 37 mg (0.04 mmol) of tert-butyl 4-({4′-[(2S)-2-{[(trans-4-{[(tert-butoxycarbonyl)-amino]methyl}cyclohexyl)-carbonyl]amino}-3-oxo-3-{[4-(2H-tetrazol-5-yl)phenyl]amino}propyl]-3-fluorobiphenyl-4-yl}carbonyl)amino]piperidine-1-carboxylate in 1.5 ml of 1,4-dioxane was admixed with 0.14 ml (0.57 mmol) of 4M hydrogen chloride in 1,4-dioxane and stirred at RT overnight. The precipitate was filtered off, washed with a little acetonitrile and dried under high vacuum. This gave 23 mg (78% of theory, 94% purity) of the title compound.

¹H NMR (400 MHz, DMSO-d₆): δ=0.82-1.01 (m, 2H), 1.11-1.32 (m, 2H), 1.41-1.52 (m, 1H), 1.55-1.64 (m, 1H), 1.66-1.84, 2.08 (m, s, 7H), 1.93-2.04 (m, 2H), 2.11-2.20 (m, 1H), 2.59-2.69 (m, 2H), 2.89-3.08 (m, 3H), 3.09-3.17 (m, 1H), 3.22-3.33 (m, 2H), 3.99-4.12 (m, 1H), 4.67-4.77 (m, 1H), 7.45 (d, 2H), 7.55-7.65 (m, 3H), 7.66-7.72 (m, 2H), 7.76-7.90 (m, 4H), 8.02 (d, 2H), 8.30 (d, 1H), 8.52 (d, 1H), 8.61-8.88 (m, 2H), 10.61 (s, 1H)

LC-MS (Method 1): Rₜ=0.52 min; MS (ESIneg): m/z=666 [M−H—HCl]⁻.

### Example 46

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-[1-(2,2,2-trifluoroethyl)piperidin-4-yl]biphenyl-4-carboxamide hydrochloride

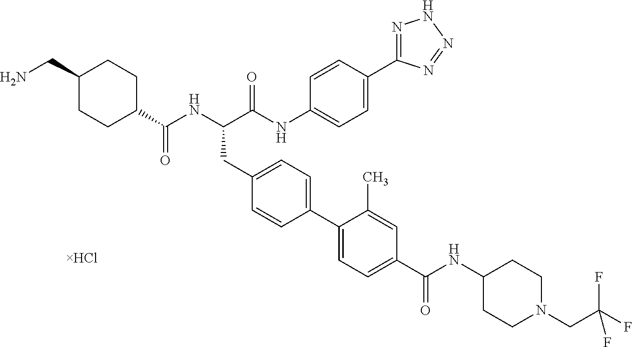

A solution of 43 mg (0.05 mmol) of tert-butyl [(trans-4-{[(2S)-3-{2′-methyl-4′-([1-(2,2,2-trifluoroethyl)piperidin-4-yl]carbamoyl}biphenyl-4-yl)-1-oxo-1-{[4-(2H-tetrazol-5-yl)phenyl]-amino}propan-2-yl]carbamoyl}cyclohexyl)methyl]carbamate trifluoroacetate in 2 ml of 1,4-dioxane was admixed with 0.17 ml (0.67 mmol) of 4M hydrogen chloride in 1,4-dioxane and stirred at RT overnight. The reaction mixture was left to stand at RT for 24 h. The precipitate was filtered off, washed with a little acetonitrile and dried under high vacuum. This gave 36 mg (95% of theory) of the title compound.

¹H NMR (400 MHz, DMSO-d₆): δ=0.83-1.01 (m, 2H), 1.09-1.33 (m, 2H), 1.40-1.53 (m, 1H), 1.53-1.62 (m, 1H), 1.63-1.93 (m, 7H), 2.10-2.28 (m, 4H), 2.59-2.69 (m, 2H), 2.90-3.19 (m, 5H), 3.77-3.92 (m, 2H), 4.69-4.80 (m, 1H), 7.18-7.30 (m, 3H), 7.39 (d, 2H), 7.64-7.86 (m, 7H), 8.01 (d, 2H), 8.20-8.38 (m, 2H), 10.52 (s, 1H).

LC-MS (Method 1): Rₜ=0.82 min; MS (ESIneg): m/z=744 [M−H—HCl]⁻.

### Example 47

### trans-4-(Aminomethyl)-N-[(2S)-1-oxo-3-[4′-(piperazin-1-ylcarbonyl)biphenyl-4-yl]-1-{[4-(2H-tetrazol-5-yl)phenyl]amino}propan-2-yl]cyclohexanecarboxamide trifluoroacetate

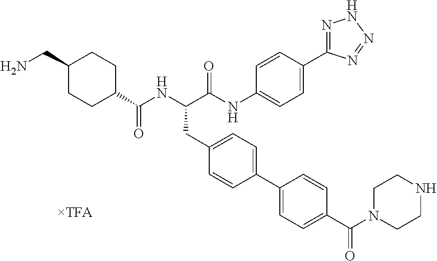

A solution of 87 mg (0.09 mmol) of tert-butyl 4-({4′-[(2S)-2-{[(trans-4-{[(tert-butoxycarbonyl)-amino]methyl}cyclohexyl)-carbonyl]amino}-3-oxo-3-{[4-(2H-tetrazol-5-yl)phenyl]amino}propyl]-biphenyl-4-yl}carbonyl)piperazine-1-carboxylate trifluoroacetate in 4 ml of 1,4-dioxane was admixed with 0.35 ml (1.38 mmol) of 4M hydrogen chloride in 1,4-dioxane and stirred at RT overnight. After the addition of a further 0.35 ml (1.38 mmol) of 4M hydrogen chloride in 1,4-dioxane, the mixture was again stirred at RT overnight. The reaction mixture was concentrated and separated by means of preparative HPLC (eluent: acetonitrile/water with 0.1% TFA (gradient)). The product-containing fractions were combined and admixed with 0.1 ml of 4M hydrogen chloride in 1,4-dioxane and the mixture was concentrated on a rotary evaporator. The residue was dried under high vacuum, dissolved in a little methanol/l % aqueous trifluoroacetic acid and separated by means of preparative HPLC (column: Sunfire C18, 5 μm, 250 mm×20 mm; eluent: water/methanol/1% aqueous trifluoroacetic acid 48:40:12; flow rate: 25 ml/min; temperature: 40° C.; UV detection: 210 nm). The product-containing fractions were concentrated on a rotary evaporator. After lyophilization overnight, 6 mg (7% of theory, 96% purity) and 7 mg (10% of theory, 94% purity) of the title compound were obtained.

LC-MS (Method 1): Rₜ=0.49 min; MS (ESIneg): m/z=634 [M−H-TFA]⁻.

### Example 48

### 4′-[(2S)-2-({[trans-4-(Aminomethyl)cyclohexyl]carbonyl}amino)-3-oxo-3-({4-[3-(trifluoromethyl)-1H-1,2,4-triazol-5-yl]phenyl}amino)propyl]-2-methyl-N-(piperidin-4-yl)biphenyl-4-carboxamide hydrochloride

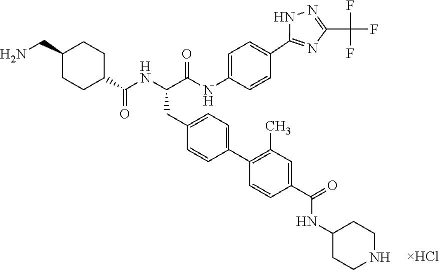

A solution of 47 mg (0.05 mmol) of tert-butyl 4-[({4′-[(2S)-2-{[(trans-4-{[(tert-butoxycarbonyl)-amino]methyl}cyclohexyl)carbonyl]amino}-3-oxo-3-({4-[3-(trifluoromethyl)-1H-1,2,4-triazol-5-yl]phenyl}amino)propyl]-2-methylbiphenyl-4-yl}carbonyl)amino]piperidine-1-carboxylate in 2 ml of tetrahydrofuran was admixed with 2.00 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and diethyl ether and dried under high vacuum. 43 mg (99% of theory, 93% purity) of the title compound were obtained.

¹H NMR (400 MHz, DMSO-d₆): δ=0.84-1.00 (m, 2H), 1.13-1.35 (m, 2H), 1.40-1.65 (m, 2H), 1.68-1.88 (m, 5H), 1.91-2.02 (m, 2H), 2.09-2.21 (m, 1H), 2.23 (s, 3H), 2.58-2.69 (m, 2H), 2.90-3.07 (m, 3H), 3.09-3.19 (m, 1H), 3.26-3.35 (m, 2H), 3.98-4.17 (m, 1H), 4.67-4.85 (m, 1H), 7.19-7.30 (m, 3H), 7.41 (d, 2H), 7.70-7.96 (m, 7H), 8.03 (d, 2H), 8.33 (d, 1H), 8.53 (d, 1H), 8.90 (br. s, 2H), 10.60 (br. s, 1H), 15.20-15.46 (br. s, 1H).

LC-MS (Method 1): Rₜ=0.66 min; MS (ESIneg): m/z=729 [M−H—HCl]⁻.

### Example 49

### 4′-[(2S)-2-({[trans-4-(Aminomethyl)cyclohexyl]carbonyl}amino)-3-{[4-(3-chloro-4H-1,2,4-triazol-5-yl)phenyl]amino}-3-oxopropyl]-2-methyl-N-(piperidin-4-yl)biphenyl-4-carboxamide hydrochloride

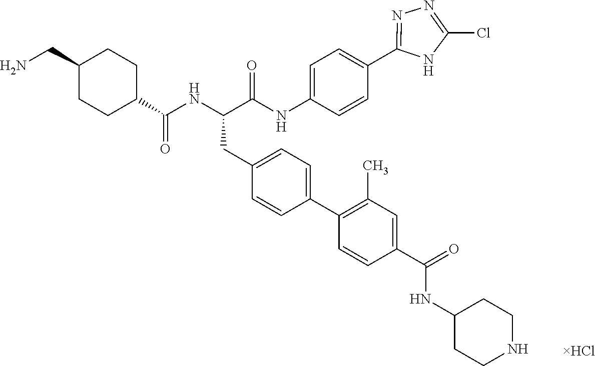

A solution of 215 mg (0.24 mmol) of tert-butyl 4-[({4′-[(2S)-2-{[(trans-4-{[(tert-butoxycarbonyl)amino]methyl}cyclohexyl)carbonyl]amino}-3-{[4-(3-chloro-4H-1,2,4-triazol-5-yl)phenyl]amino}-3-oxopropyl]-2-methylbiphenyl-4-yl}carbonyl)amino]piperidine-1-carboxylate in 4 ml of 1,4-dioxane was admixed with 2.00 ml (8.00 mmol) of 4M hydrogen chloride in 4 ml of 1,4-dioxane and stirred at RT for 16 h. The solvent was removed on a rotary evaporator, and the residue was stirred in acetonitrile and filtered. The solid formed was washed with acetonitrile and dried under high vacuum. 110 mg (57% of theory) of the title compound were obtained.

¹H NMR (400 MHz, DMSO-d₆): δ=0.94 (m, 6.40 Hz, 3H), 1.08-1.34 (m, 2H), 1.48 (m, 3H), 1.67-1.87 (m, 3H), 1.97 (m, 2H), 2.10-2.26 (m, 4H), 2.62 (m, 2H), 2.98 (m, 3H), 3.08-3.19 (m, 1H), 3.31 (d, 2H), 4.01-4.13 (m, 1H), 4.69-4.79 (m, 1H), 7.20-7.34 (m, 3H), 7.40 (d, 2H), 7.70-7.83 (m, 4H), 7.92 (m, 5H), 8.30 (d, 1H), 8.50 (d, 1H), 8.74-9.15 (br. s., 2H), 10.57 (br. s., 1H), 14.81 (br. s, 1H).

LC-MS (Method 1): Rₜ=0.67 min; MS (ESIneg): m/z=695 [M−H—HCl]⁻.

### Example 50

### 4′-{(2S)-2-({[trans-4-(Aminomethyl)cyclohexyl]carbonyl}amino)-3-[(2-methyl-1H-benzimidazol-6-yl)amino]-3-oxopropyl}-2-methyl-N-(piperidin-4-yl)biphenyl-4-carboxamide hydrochloride

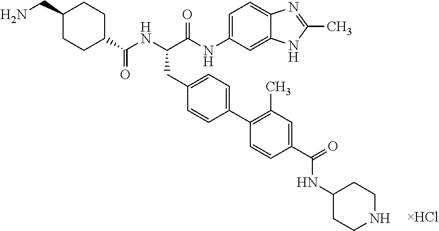

A solution of 19 mg (0.02 mmol) of tert-butyl 4-{[(4′-{(2S)-2-{[(trans-4-{[(tert-butoxycarbonyl)amino]methyl}cyclohexyl)carbonyl]amino}-3-[(2-methyl-1H-benzimidazol-6-yl)amino]-3-oxopropyl}-2-methylbiphenyl-4-yl)carbonyl]amino}piperidine-1-carboxylate in 2 ml of tetrahydrofuran was admixed with 1.00 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 14 mg (86% of theory) of the title compound were obtained.

¹H NMR (400 MHz, DMSO-d₆): δ=0.81-1.00 (m, 2H), 1.17 (s, 2H), 1.40-1.86 (m, 8H), 1.90-2.01 (m, 2H), 2.23 (m, 4H), 2.58-2.67 (m, 2H), 2.78 (s, 3H), 2.86-3.20 (m, 4H), 3.26-3.35 (m, 2H), 3.39-3.42 (m, 1H), 3.64 (t, 1H), 3.99-4.15 (m, 1H), 4.65-4.82 (m, 1H), 7.16-7.30 (m, 3H), 7.42 (d, 2H), 7.57-7.65 (m, 1H), 7.67-7.82 (m, 3H), 7.92 (br. s, 3H), 8.28 (m, 2H), 8.43-8.57 (d, 1H), 8.92 (br. s, 2H), 10.70 (s, 1H), 14.83 (br. s, 1H).

LC-MS (Method 1): Rₜ=0.43 min; MS (ESIneg): m/z=648 [M−H—HCl]⁻.

### Example 51

### 4′-[(2S)-2-({[trans-4-(Aminomethyl)cyclohexyl]carbonyl}amino)-3-oxo-3-{[2-(pyridin-2-yl)-1H-benzimidazol-5-yl]amino}propyl]-2-methyl-N-(piperidin-4-yl)biphenyl-4-carboxamide hydrochloride

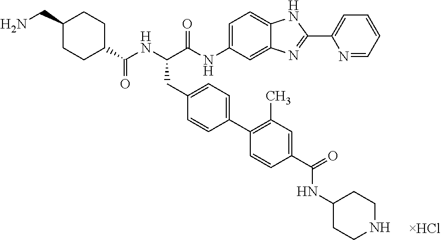

A solution of 47 mg (0.05 mmol) of tert-butyl 4-[({4′-[(2S)-2-{[(trans-4-{[(tert-butoxycarbonyl)amino]methyl}cyclohexyl)carbonyl]amino}-3-oxo-3-{[2-(pyridin-2-yl)-1H-benzimidazol-5-yl]amino}propyl]-2-methylbiphenyl-4-yl}carbonyl)amino]piperidine-1-carboxylate in 2 ml of tetrahydrofuran was admixed with 1.00 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 37 mg (82% of theory, 90% purity) of the title compound were obtained.

¹H NMR (400 MHz, DMSO-d₆): δ=0.79-1.04 (m, 2H), 1.18 (m, 2H), 1.41-1.64 (m, 2H), 1.76 (dd, 6H), 1.91-2.01 (m, 1H), 2.24 (m, 4H), 2.62 (m, 2H), 2.90-3.08 (m, 4H), 3.13-3.23 (m, 1H), 3.35 (m, 2H), 4.03 (m, 1H), 4.67-4.84 (m, 1H), 7.25 (m, 4H), 7.44 (d, 1H), 7.63-7.83 (m, 6H), 7.96 (br. m, 4H), 8.15-8.25 (m, 1H), 8.30-8.41 (m, 2H), 8.51 (d, 1H), 8.65 (d, 1H), 8.88 (d, 1H), 9.02 (br. s, 2H), 10.74 (br. s, 1H).

LC-MS (Method 1): Rₜ=0.55 min; MS (ESIneg): m/z=711 [M−H—HCl]⁻.

### Example 52

### 4′-[(2S)-2-({[trans-4-(Aminomethyl)cyclohexyl]carbonyl}amino)-3-oxo-3-{[2-(trifluoromethyl)-1H-benzimidazol-6-yl]amino}propyl]-2-methyl-N-(piperidin-4-yl)biphenyl-4-carboxamide hydrochloride

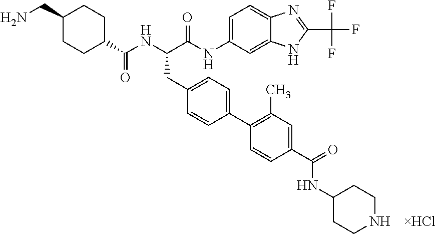

A solution of 53 mg (0.06 mmol) of tert-butyl 4-[({4′-[(2S)-2-{[(trans-4-{[(tert-butoxycarbonyl)amino]methyl}cyclohexyl)carbonyl]amino}-3-oxo-3-{[2-(trifluoromethyl)-1H-benzimidazol-6-yl]amino}propyl]-2-methylbiphenyl-4-yl}carbonyl)amino]piperidine-1-carboxylate in 2 ml of tetrahydrofuran was admixed with 1.00 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 38 mg (80% of theory) of the title compound were obtained.

¹H NMR (400 MHz, DMSO-d₆): δ=0.81-1.00 (m, 2H), 1.09 (m, 2H), 1.41-1.63 (m, 2H), 1.65-2.01 (m, 7H), 2.11-2.27 (m, 4H), 2.62 (m, 2H), 2.87-3.18 (m, 4H), 3.24-3.42 (m, 3H), 4.01-4.14 (m, 1H), 4.67-4.84 (m, 1H), 7.13-7.28 (m, 3H), 7.40 (m, 3H), 7.62-7.80 (m, 3H), 7.92 (br. s., 3H), 8.17-8.35 (m, 2H), 8.52 (d, 1H), 8.93 (br. s, 2H), 10.44 (s, 1H).

LC-MS (Method 1): Rₜ=0.59 min; MS (ESIneg): m/z=702 [M−H—HCl]⁻.

### Example 53

### 4′-{(2S)-2-({[trans-4-(Aminomethyl)cyclohexyl]carbonyl}amino)-3-oxo-3-[(2-oxo-2,3-dihydro-1H-benzimidazol-5-yl)amino]propyl}-2-methyl-N-(piperidin-4-yl)biphenyl-4-carboxamide hydrochloride

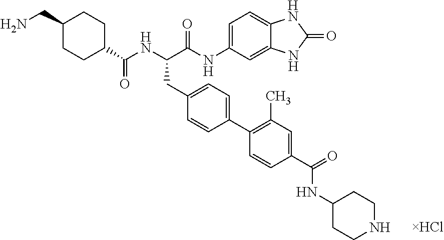

A solution of 69 mg (0.08 mmol) of tert-butyl 4-{[(4′-{(2S)-2-{[(trans-4-{[(tert-butoxycarbonyl)amino]methyl}cyclohexyl)carbonyl]amino}-3-oxo-3-[(2-oxo-2,3-dihydro-1H-benzimidazol-5-yl)amino]propyl}-2-methylbiphenyl-4-yl)carbonyl]amino}piperidine-1-carboxylate in 2 ml of tetrahydrofuran was admixed with 1.00 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 55 mg (85% of theory, 91% purity) of the title compound were obtained.

¹H NMR (400 MHz, DMSO-d₆): δ=0.81-0.99 (m, 2H), 1.02-1.33 (m, 4H), 1.44-1.61 (m, 2H), 1.63-1.89 (m, 5H), 1.90-2.02 (m, 2H), 2.04-2.30 (m, 4H), 2.58-2.66 (m, 2H), 2.98 (m, 3H), 3.31 (m, 2H), 4.03 (m, 1H), 4.58-4.77 (m, 1H), 6.84 (d, 1H), 7.07 (dd, 1H), 7.16-7.28 (m, 3H), 7.34-7.52 (m, 3H), 7.68-8.08 (m, 5H), 8.25 (d, 1H), 8.53 (d, 1H), 9.01 (br. s., 2H), 10.11 (s, 1H), 10.49-10.63 (m, 2H).

LC-MS (Method 1): Rₜ=0.52 min; MS (ESIneg): m/z=650 [M−H—HCl]⁻.

### Example 54

### 4′-[(2S)-2-({[trans-4-(Aminomethyl)cyclohexyl]carbonyl}amino)-3-({4-[3-(methoxymethyl)-4H-1,2,4-triazol-5-yl]phenyl}amino)-3-oxopropyl]-2-methyl-N-(piperidin-4-yl)biphenyl-4-carboxamide hydrochloride

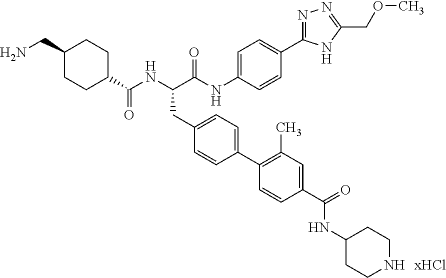

A solution of 39 mg (0.04 mmol) of tert-butyl 4-[({4′-[(2S)-2-{[(trans-4-{[(tert-butoxycarbonyl)amino]methyl}cyclohexyl)carbonyl]amino}-3-({4-[3-(methoxymethyl)-4H-1,2,4-triazol-5-yl]phenyl}amino)-3-oxopropyl]-2-methylbiphenyl-4-yl}carbonyl)amino]piperidine-1-carboxylate in 2 ml of tetrahydrofuran was admixed with 2.00 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 31 mg (79% of theory, 85% purity) of the title compound were obtained.

¹H NMR (400 MHz, DMSO-d₆): δ=0.78-1.01 (m, 2H), 1.03-1.35 (m, 2H), 1.39-1.64 (m, 2H), 1.65-2.02 (m, 8H), 2.04-2.30 (m, 4H), 2.57-2.69 (m, 2H), 2.79-3.19 (m, 5H), 3.25-3.41 (m, 4H), 4.02-4.13 (m, 1H), 4.44-4.59 (m, 2H), 4.66-4.81 (m, 1H), 7.16-7.45 (m, 6H), 7.64-8.01 (m, 8H), 8.30 (d, 1H), 8.51 (d, 1H), 8.88 (br. s, 2H), 10.45 (br. s, 1H).

LC-MS (Method 1): Rₜ=0.57 min; MS (ESIneg): m/z=705 [M−H—HCl]⁻.

### Example 55

### 4′-{(2S)-2-({[trans-4-(Aminomethyl)cyclohexyl]carbonyl}amino)-3-oxo-3-[(2-oxo-2,3-dihydro-1,3-benzoxazol-6-yl)amino]propyl}-2-methyl-N-(piperidin-4-yl)biphenyl-4-carboxamide hydrochloride

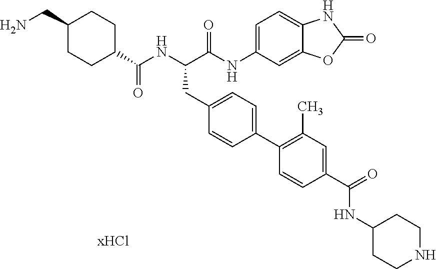

A solution of 93 mg (0.1 mmol) of tert-butyl 4-{[(4′-{(2S)-2-{[(trans-4-{[(tert-butoxycarbonyl)amino]methyl}cyclohexyl)carbonyl]amino}-3-oxo-3-[(2-oxo-2,3-dihydro-1,3-benzoxazol-6-yl)amino]propyl}-2-methylbiphenyl-4-yl)carbonyl]amino}piperidine-1-carboxylate in 2 ml of 1,4-dioxane was admixed with 2 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and diethyl ether and dried under high vacuum. 72 mg (82% of theory, 90% purity) of the title compound were obtained.

¹H NMR (400 MHz, DMSO-d₆): δ=0.74-1.01 (m, 2H), 1.13-1.34 (m, 2H), 1.41-1.63 (m, 2H), 1.67-1.87 (m, 5H), 1.90-2.02 (m, 3H), 2.09-2.19 (m, 1H), 2.57-2.66 (m, 2H), 2.98 (m, 3H), 3.07-3.18 (m, 1H), 3.31 (m, 2H), 3.57 (s, 3H), 4.04-4.13 (m, 1H), 4.64-4.80 (m, 1H), 7.15-7.31 (m, 5H), 7.39 (d, 2H), 7.61 (s, 1H), 7.74 (d, 1H), 7.80 (s, 1H), 7.95 (br. s., 3H), 8.25-8.36 (m, 1H), 8.53 (d, 1H), 8.99 (br. s., 2H), 10.36 (s, 1H), 11.67 (s, 1H).

LC-MS (Method 1): Rₜ=0.56 min; MS (ESIneg): m/z=651 [M−H—HCl]⁻.

### Example 56

### 4′-[(2S)-2-({[trans-4-(Aminomethyl)cyclohexyl]carbonyl}amino)-3-{[4-(3-methyl-4H-1,2,4-triazol-5-yl)phenyl]amino}-3-oxopropyl]-2-methyl-N-(piperidin-4-yl)biphenyl-4-carboxamide hydrochloride

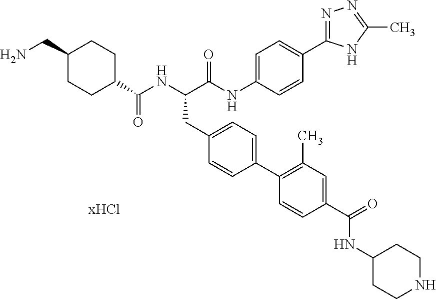

A solution of 93 mg (0.1 mmol) of tert-butyl 4-[({4′-[(2S)-2-{[(trans-4-{[(tert-butoxycarbonyl)amino]methyl}cyclohexyl)carbonyl]amino}-3-{[4-(3-methyl-4H-1,2,4-triazol-5-yl)phenyl]amino}-3-oxopropyl]-2-methylbiphenyl-4-yl}carbonyl)amino]piperidine-1-carboxylate in 2 ml of 1,4-dioxane was admixed with 2 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and diethyl ether and dried under high vacuum. 71 mg (70% of theory) of the title compound were obtained.

¹H NMR (400 MHz, DMSO-d₆): δ=0.81-1.01 (m, 2H), 1.20-1.34 (m, 1H), 1.42-1.61 (m, 2H), 1.67-1.84 (m, 5H), 1.92-2.00 (m, 2H), 2.10-2.19 (m, 1H), 2.23 (s, 3H), 2.39 (s, 3H), 2.59-2.68 (m, 2H), 2.88-3.18 (m, 3H), 3.26-3.37 (m, 2H), 3.78-3.82 (m, 1H), 4.00-4.14 (m, 1H), 4.43-4.57 (m, 2H), 4.67-4.81 (m, 1H), 6.57-6.81 (m, 1H), 7.26 (m, 5H), 7.63-8.01 (m, 8H), 8.21-8.34 (m, 1H), 8.43-8.57 (m, 1H), 8.83 (br. s, 2H), 10.40 (s, 1H), 10.85 (br. s, 1H), 11.52 (br. s, 1H).

LC-MS (Method 1): Rₜ=0.52 min; MS (ESIneg): m/z=675 [M−H—HCl]⁻.

### Example 57

### 4′-[(2S)-2-({[trans-4-(Aminomethyl)cyclohexyl]carbonyl}amino)-3-oxo-3-{[2-(pentafluoroethyl)-1H-benzimidazol-5-yl]amino}propyl]-2-methyl-N-(piperidin-4-yl)biphenyl-4-carboxamide hydrochloride

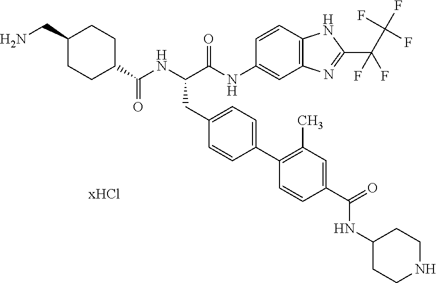

A solution of 76.5 mg (0.08 mmol) of tert-butyl 4-[({4′-[(2S)-2-{[(trans-4-{[(tert-butoxycarbonyl)amino]methyl}cyclohexyl)carbonyl]amino}-3-oxo-3-{[2-(pentafluoroethyl)-1H-benzimidazol-5-yl]amino}propyl]-2-methylbiphenyl-4-yl}carbonyl)amino]piperidine-1-carboxylate in 2 ml of 1,4-dioxane was admixed with 1 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and diethyl ether and dried under high vacuum. 62 mg (90% of theory) of the title compound were obtained.

¹H NMR (400 MHz, DMSO-d₆): δ=0.81-1.00 (m, 2H), 1.12-1.33 (m, 2H), 1.40-2.05 (m, 9H), 2.11-2.19 (m, 1H), 2.22-2.28 (m, 3H), 2.62 (m, 2H), 3.00 (d, 3H), 3.09-3.20 (m, 1H), 3.31 (m, 2H), 3.98-4.14 (m, 1H), 4.78 (m, 2H), 7.14-7.30 (m, 3H), 7.36-7.53 (m, 3H), 7.63-7.85 (m, 3H), 7.98 (br. s., 3H), 8.25 (d, 1H), 8.32 (d, 1H), 8.53 (d, 1H), 9.03 (br. s., 2H), 10.50 (s, 1H).

LC-MS (Method 1): Rₜ=0.66 min; MS (ESIneg): m/z=752 [M−H—HCl]⁻.

### Example 58

### 4′-[(2S)-2-({[trans-4-(Aminomethyl)cyclohexyl]carbonyl}amino)-3-{[4-(3-isobutyl-4H-1,2,4-triazol-5-yl)phenyl]amino}-3-oxopropyl]-2-methyl-N-(piperidin-4-yl)biphenyl-4-carboxamide hydrochloride

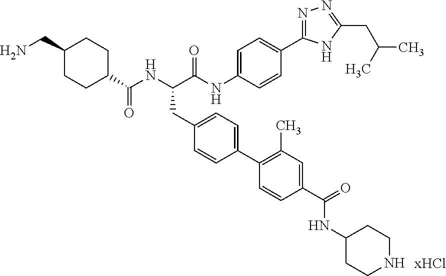

A solution of 127 mg (0.14 mmol) of tert-butyl 4-[({4′-[(2S)-2-{[(trans-4-{[(tert-butoxycarbonyl)amino]methyl}cyclohexyl)carbonyl]amino}-3-{[4-(3-isobutyl-4H-1,2,4-triazol-5-yl)phenyl]amino}-3-oxopropyl]-2-methylbiphenyl-4-yl}carbonyl)amino]piperidine-1-carboxylate in 2 ml of 1,4-dioxane was admixed with 2 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and diethyl ether and dried under high vacuum. 96 mg (88% of theory) of the title compound were obtained.

¹H NMR (400 MHz, DMSO-d₆): δ=0.83-0.93 (m, 2H), 0.98 (d, 6H), 1.07-1.35 (m, 2H), 1.43-1.63 (m, 2H), 1.66-1.89 (m, 4H), 1.89-2.01 (m, 2H), 2.08-2.20 (m, 1H), 2.23 (s, 3H), 2.26-2.34 (m, 1H), 2.62 (m, 2H), 3.00 (d, 4H), 3.11-3.21 (m, 1H), 3.25-3.36 (m, 2H), 3.99-4.17 (m, 1H), 4.63-4.85 (m, 1H), 7.18-7.29 (m, 3H), 7.42 (d, 2H), 7.58-7.68 (m, 1H), 7.69-7.82 (m, 3H), 7.96 (br. s., 3H), 8.31 (m, 2H), 8.52 (d, 1H), 8.95 (br. s, 2H), 10.74 (s, 1H), 15.06 (br. s, 2H).

LC-MS (Method 1): Rₜ=0.52 min; MS (ESIneg): m/z=690 [M−H—HCl]⁻.

### Example 59

### 4′-[(2S)-2-({[trans-4-(Aminomethyl)cyclohexyl]carbonyl}amino)-3-oxo-3-{[2-(pyridin-3-yl)-1H-benzimidazol-6-yl]amino}propyl]-2-methyl-N-(piperidin-4-yl)biphenyl-4-carboxamide hydrochloride

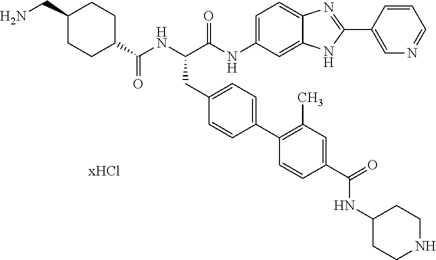

A solution of 131 mg (0.14 mmol) of tert-butyl 4-[({4′-[(2S)-2-{[(trans-4-{[(tert-butoxycarbonyl)amino]methyl}cyclohexyl)carbonyl]amino}-3-oxo-3-{[2-(pyridin-3-yl))-1H-benzimidazol-6-yl]amino}propyl]-2-methylbiphenyl-4-yl}carbonyl)amino]piperidine-1-carboxylate in 2 ml of 1,4-dioxane was admixed with 2 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and diethyl ether and dried under high vacuum. 111 mg (92% of theory) of the title compound were obtained.

¹H NMR (400 MHz, DMSO-d₆): δ=0.81-1.00 (m, 2H), 1.06-1.37 (m, 2H), 1.44-1.62 (m, 2H), 1.68-1.87 (m, 5H), 1.90-2.02 (m, 2H), 2.11-2.19 (m, 1H), 2.24 (s, 3H), 2.58-2.68 (m, 2H), 2.91-3.07 (m, 3H), 3.12-3.23 (m, 1H), 3.25-3.36 (m, 2H), 4.00-4.13 (m, 1H), 4.72-4.83 (m, 1H), 7.13-7.35 (m, 3H), 7.43 (d, 2H), 7.61-8.07 (m, 8H), 8.27-8.44 (m, 2H), 8.53 (d, 1H), 8.89 (m, 5H), 9.57 (s, 1H), 10.75 (s, 1H).

LC-MS (Method 1): Rₜ=0.56 min; MS (ESIneg): m/z=711 [M−H—HCl]⁻.

### Example 60

### 4′-[(2S)-2-({[trans-4-(Aminomethyl)cyclohexyl]carbonyl}amino)-3-oxo-3-{[2-(1H-pyrazol-1-yl)-1H-benzimidazol-6-yl]amino}propyl]-2-methyl-N-(piperidin-4-yl)biphenyl-4-carboxamide hydrochloride

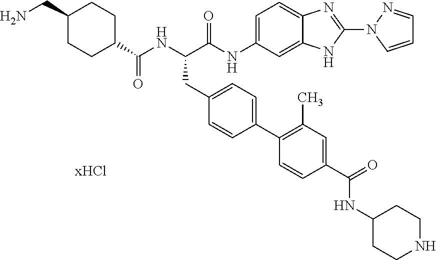

A solution of 142 mg (0.16 mmol) of tert-butyl 4-[({4′-[(2S)-2-{[(trans-4-{[(tert-butoxycarbonyl)amino]methyl}cyclohexyl)carbonyl]amino}-3-oxo-3-{[2-(1H-pyrazol-1-yl)-1H-benzimidazol-6-yl]amino}propyl]-2-methylbiphenyl-4-yl}carbonyl)amino]piperidine-1-carboxylate in 2 ml of 1,4-dioxane was admixed with 2 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 97 mg (73% of theory, 92% purity) of the title compound were obtained.

¹H NMR (400 MHz, DMSO-d₆): δ=0.82-1.00 (m, 2H), 1.13-1.35 (m, 2H), 1.44-1.61 (m, 2H), 1.66-1.90 (m, 4H), 1.97 (m, 2H), 2.17 (br. s., 1H), 2.24 (s, 3H), 2.58-2.66 (m, 2H), 2.90-3.06 (m, 3H), 3.15 (d, 1H), 3.31 (m, 2H), 3.99-4.17 (m, 1H), 4.77 (m, 1H), 6.66-6.82 (m, 1H), 7.18-7.34 (m, 3H), 7.37-7.58 (m, 3H), 7.68-7.86 (m, 2H), 7.95-8.17 (m, 4H), 8.32 (d, 1H), 8.51-8.78 (m, 2H), 9.12 (br. s., 2H), 10.43 (s, 1H).

LC-MS (Method 1): Rₜ=0.58 min; MS (ESIneg): m/z=700 [M−H—HCl]⁻.

### Example 61

### 4′-[(2S)-2-({[trans-4-(Aminomethyl)cyclohexyl]carbonyl}amino)-3-oxo-3-{[4-(2H-tetrazol-5-yl)phenyl]amino}propyl]-2′-fluoro-2-methyl-N-(piperidin-4-yl)biphenyl-4-carboxamide hydrochloride

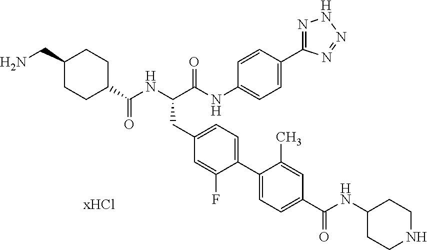

A solution of 82 mg (0.08 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2′-fluoro-2-methylbiphenyl-4-yl}carbonyl)amino]piperidine-1-carboxylate in 3 ml of 1,4-dioxane was admixed with 0.3 ml (1.2 mmol) of 4M hydrogen chloride in 1,4-dioxane and stirred at RT for 16 h. The solid formed was filtered off, washed with acetonitrile and dried under high vacuum. This gave 55 mg (79% of theory) of the title compound.

¹H NMR (400 MHz, DMSO-d₆): δ=0.86-1.00 (m, 2H), 1.12-1.33 (m, 2H), 1.43-1.52 (m, 1H), 1.55-1.62 (m, 1H), 1.70-1.85 (m, 6H), 1.93-2.01 (m, 2H), 2.12 (s, 3H), 2.14-2.20 (m, 1H), 2.60-2.68 (m, 2H), 2.93-3.07 (m, 4H), 3.18 (dd, 1H), 3.27-3.35 (m, 3H), 4.02-4.12 (m, 1H), 4.72-4.81 (m, 1H), 7.18-7.32 (m, 5H), 7.74 (d, 1H), 7.79 (s, 1H), 7.82-7.93 (m, 6H), 8.02 (d, 2H), 8.33 (d, 1H), 8.53 (d, 1H), 8.73-8.94 (m, 3H), 10.63 (s, 1H).

LC-MS (Method 1): Rₜ=0.55 min; MS (ESIneg): m/z=680.5 [M−H—HCl]⁻.

### Example 62

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-cyclopropylbiphenyl-4-carboxamide hydrochloride

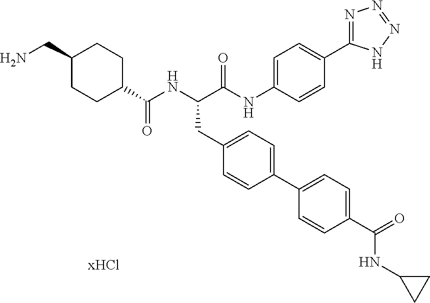

A suspension of 40 mg (0.06 mmol) of tert-butyl [(trans-4-{[(2S)-3-[4′-(cyclopropylcarbamoyl)biphenyl-4-yl]-1-oxo-1-{[4-(1H-tetrazol-5-yl)phenyl]amino}-propan-2-yl]carbamoyl}cyclohexyl)methyl]carbamate in 2.0 ml of 1,4-dioxane was admixed with 0.1 ml (0.4 mmol) of 4M hydrogen chloride in 1,4-dioxane and stirred at RT overnight. After adding a further 0.06 ml (0.22 mmol) of 4M hydrogen chloride in 1,4-dioxane and stirring for 48 h, the reaction mixture was admixed with 5 ml of acetonitrile, and the precipitate formed was filtered off with suction and dried under high vacuum. This gave 27 mg (71% of theory) of the title compound.

1H NMR (300 MHz, DMSO-d6): δ=0.55 (m, 2H), 0.62-0.71 (m, 2H), 0.78-0.98 (m, 2H), 1.06-1.31 (m, 2H), 1.34-1.49 (m, 1H), 1.52-1.62 (m, 1H), 1.64-1.78 (m, 3H), 2.04-2.17 (m, 1H), 2.54-2.64 (m, 2H), 2.77-2.85 (m, 1H), 2.90 (dd, 1H), 3.09 (dd, 1H), 4.62-4.75 (m, 1H), 7.39 (d, 2H), 7.63 (d, 2H), 7.69 (m, 5H), 7.80 (d, 2H), 7.86 (d, 2H), 7.98 (d, 2H), 8.23 (d, 1H), 8.44 (d, 1H), 10.53 (s, 1H).

LC-MS (Method 4): Rₜ=0.76 min; MS (ESIpos): m/z=607.6 [M+H—HCl]⁺.

### Example 63

### 4′-[(2S)-2-({[trans-4-(Aminomethyl)cyclohexyl]carbonyl}amino)-3-{[3-fluoro-4-(2H-tetrazol-5-yl)phenyl]amino}-3-oxopropyl]-N-[4-(dimethylamino)cyclohexyl]-2-methylbiphenyl-4-carboxamide hydrochloride

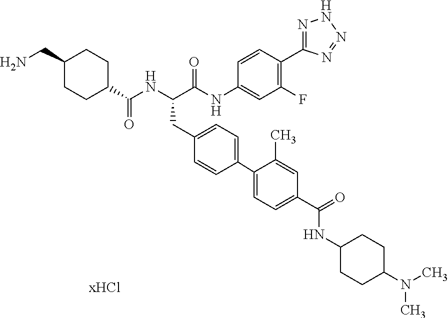

A suspension of 38 mg (0.045 mmol) of tert-butyl [(trans-4-{[(2S)-3-(4′-{[4-(dimethylamino)cyclohexyl]carbamoyl}-2′-methylbiphenyl-1-yl)-1-{[3-fluoro-4-(2H-tetrazol-5-yl)phenyl]amino}-1-oxopropan-2-yl]carbamoyl}cyclohexyl)methyl]carbamate in 2.8 ml of dichloromethane was admixed with 0.11 ml (0.45 mmol) of 4M hydrogen chloride in dioxane and stirred at RT overnight. Subsequently, the mixture was admixed with acetonitrile, and the residue was filtered off and dried under reduced pressure. This gave 23 mg (63% of theory) of the title compound.

¹H NMR (300 MHz, DMSO-d₆): δ=0.82-1.00 (m, 3H), 1.08-1.33 (m, 3H), 1.36-1.65 (m, 7H), 1.68-1.89 (m, 7H), 1.90-2.02 (m, 3H), 2.07 (m, 3H), 2.21 (m, 3H), 2.57-2.66 (m, 3H), 2.70 (m, 6H), 2.86-3.02 (m, 2H), 3.07-3.21 (m, 3H), 4.02-4.11 (m, 1H), 4.65-4.78 (m, 1H), 7.24 (m, 3H), 7.34-7.44 (m, 2H), 7.50-7.58 (m, 2H), 7.65-7.76 (m, 3H), 7.79-7.92 (m, 4H), 7.96-8.11 (m, 2H), 8.26-8.37 (m, 1H), 10.25-10.44 (m, 1H), 10.77-10.90 (m, 1H).

LC-MS (Method 4): Rₜ=0.67 min; MS (ESIpos): m/z=724.4 [M+H—HCl]⁺.

### Example 64

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-[(3R)-2-oxoazepan-3-yl]biphenyl-4-carboxamide hydrochloride

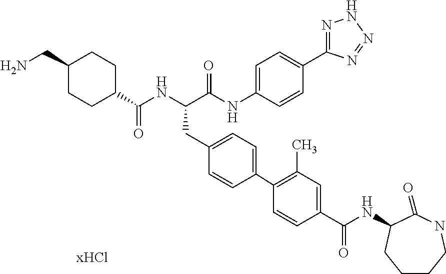

To a solution of 72 mg (0.08 mmol) of tert-butyl [(trans-4-{[(2S)-3-{2′-methyl-4′-{[(3S)-2-oxoazepan-3-yl]carbamoyl}biphenyl-4-yl)-1-oxo-1-{[4-(2H-tetrazol-5-yl)phenyl]amino}propan-2-yl]carbamoyl}cyclohexyl)methyl]carbamate in 2 ml of dioxane was added 0.3 ml (1.2 mmol) of 4M hydrogen chloride in dioxane. The mixture was stirred at RT for 16 h. The precipitated product was filtered off with suction, washed with acetonitrile and dried under high vacuum. 57 mg (87% of theory, 92% purity) of the title compound were obtained.

¹H NMR (400 MHz, DMSO-d₆): δ=0.84-0.98 (m, 2H), 1.10-1.31 (m, 3H), 1.41-1.63 (m, 4H), 1.73 (m, 6H), 1.86-1.98 (m, 2H), 2.15 (t, 1H), 2.24 (s, 3H), 2.60-2.68 (m, 2H), 2.91-3.01 (m, 1H), 3.06-3.18 (m, 2H), 3.20-3.30 (m, 1H), 4.63 (dd, 1H), 4.72-4.81 (m, 1H), 7.23-7.30 (m, 3H), 7.41 (d, 2H), 7.72 (d, 1H), 7.77-7.86 (m, 6H), 7.89 (t, 1H), 8.02 (d, 2H), 8.22 (d, 1H), 8.29 (d, 1H), 10.56 (s, 1H).

LC-MS (Method 1): Rₜ=0.69 min; MS (ESIneg): m/z=692 [M−H—HCl]⁻.

### Example 65

### 4-[5-({(2S)-2-({[trans-4-(Aminomethyl)cyclohexyl]carbonyl}amino)-3-[2′-methyl-4′-(piperidin-4-ylcarbamoyl)biphenyl-4-yl]propanoyl}amino)-1H-benzimidazol-2-yl]-2,2,3,3,4,4-hexafluorobutanoic acid hydrochloride

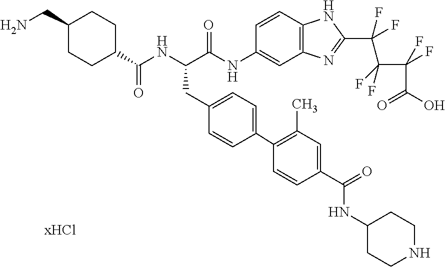

A solution of 175.6 mg (0.17 mmol) of 4-(5-{[(2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoyl]amino}-1H-benzimidazol-2-yl)-2,2,3,3,4,4-hexafluorobutanoic acid in 4 ml of 1,4-dioxane was admixed with 1 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 139.4 mg (88% of theory) of the title compound were obtained.

¹H NMR (400 MHz, DMSO-d₆): δ=0.82-1.00 (m, 2H), 1.08-1.36 (m, 2H), 1.40-1.62 (m, 2H), 1.65-1.88 (m, 6H), 1.91-2.02 (m, 2H), 2.10-2.28 (m, 5H), 2.58-2.68 (m, 2H), 2.89-3.07 (m, 3H), 3.10-3.19 (m, 1H), 3.26-3.36 (m, 2H), 3.97-4.14 (m, 1H), 4.69-4.86 (m, 1H), 7.14-7.49 (m, 6H), 7.63-8.04 (m, 6H), 8.15-8.33 (m, 2H), 8.43-8.58 (m, 1H), 8.94 (br. s, 2H), 10.35-10.49 (m, 1H).

LC-MS (Method 1): Rₜ=0.59 min; MS (ESIneg): m/z=828 [M−H—HCl]⁻.

### Example 66

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[1,3-bis(dimethylamino)propan-2-yl]-2-methylbiphenyl-4-carboxamide hydrochloride

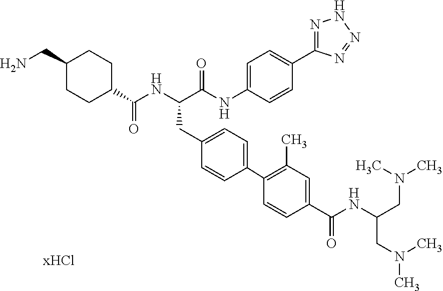

To a solution of 102 mg (0.11 mmol) of tert-butyl [(trans-4-{[(2S)-3-(4′-{([1,3-bis(dimethylamino)propan-2-yl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate in 3 ml of dioxane was added 0.41 ml (1.65 mmol) of 4M hydrogen chloride in dioxane. The mixture was stirred at RT for 16 h. The precipitated product was filtered off with suction, washed with acetonitrile and dried under high vacuum. 73 mg (82% of theory) of the title compound were obtained.

¹H NMR (400 MHz, DMSO-d₆): δ=0.85-0.99 (m, 2H), 1.13-1.34 (m, 2H), 1.42-1.54 (m, 1H), 1.55-1.64 (m, 1H), 1.71-1.82 (m, 3H), 2.08 (s, 2H), 2.12-2.21 (m, 1H), 2.24 (s, 3H), 2.60-2.68 (m, 2H), 2.90-3.03 (m, 1H), 3.09-3.19 (m, 1H), 3.36-3.54 (m, 14H), 4.70-4.90 (m, 2H), 7.20-7.31 (m, 3H), 7.40 (d, 2H), 7.79-7.91 (m, 6H), 7.94 (s, 1H), 8.03 (d, 2H), 8.33 (d, 1H), 9.08 (d, 1H), 10.02-10.22 (m, 2H), 10.59 (s, 1H).

LC-MS (Method 1): Rₜ=0.52 min; MS (ESIneg): m/z=709 [M−H—HCl]⁻.

### Example 67

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[4-(dimethylamino)cyclohexyl]-2-methoxybiphenyl-4-carboxamide hydrochloride

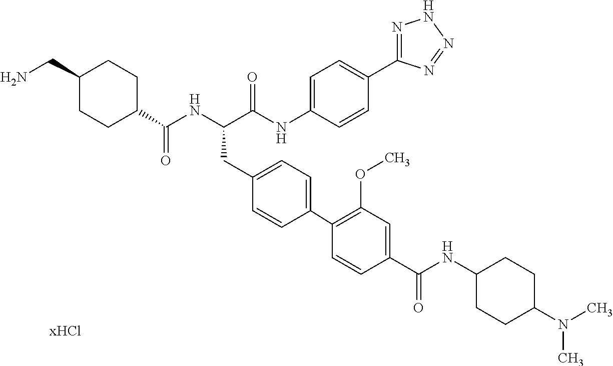

To a solution of 65 mg (0.07 mmol) of tert-butyl [(trans-4-{[(2S)-3-(4′-{[4-(dimethylamino)-cyclohexyl]carbamoyl}-2′-methoxybiphenyl-4-yl)-1-oxo-1-{[4-(2H-tetrazol-5-yl)phenyl]amino}-propan-2-yl]carbamoyl}cyclohexyl)methyl]carbamate trifluoroacetate in 2.5 ml of dioxane was added 0.26 ml (1.04 mmol) of 4M hydrogen chloride in dioxane. The mixture was stirred at RT for 16 h. The precipitated product was filtered off with suction, washed with acetonitrile and dried under high vacuum. 46 mg (80% of theory) of the title compound were obtained.

¹H NMR (400 MHz, DMSO-d₆): δ=0.86-1.00 (m, 2H), 1.13-1.34 (m, 2H), 1.40-1.52 (m, 2H), 1.62 (m, 3H), 1.70-1.89 (m, 4H), 1.94-2.11 (m, 4H), 2.12-2.21 (m, 1H), 2.64 (m, 2H), 2.71 (br. s., 3H), 2.72 (br. s., 3H), 2.87-2.98 (m, 1H), 3.07-3.23 (m, 2H), 3.57 (s, 3H), 3.82 (m, 4H), 4.67-4.76 (m, 1H), 7.31-7.47 (m, 6H), 7.48-7.55 (m, 2H), 7.84 (m, 5H), 8.03 (d, 2H), 8.25-8.33 (m, 1H), 8.35-8.42 (m, 1H), 10.15-10.31 (m, 1H), 10.58 (br. s., 1H).

LC-MS (Method 1): Rₜ=0.69 min; MS (ESIneg): m/z=722 [M−H—HCl]⁻.

### Example 68

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-N-[4-(dimethylamino)cyclohexyl]-2-methylbiphenyl-4-carboxamide hydrochloride

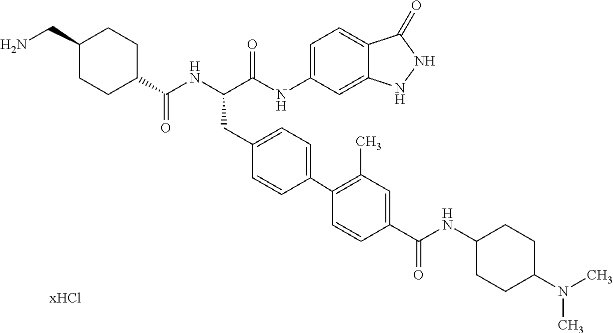

To a solution of 80 mg (0.09 mmol) of tert-butyl {[trans-4-({(2S)-3-(4′-{[4-(dimethylamino)cyclohexyl]carbamoyl}-2′-methylbiphenyl-4-yl)-1-oxo-1-[(3-oxo-2,3-dihydro-1H-indazol-6-yl)amino]propan-2-yl}carbamoyl)cyclohexyl]methyl}-carbamate trifluoroacetate in 2 ml of dioxane were added 330 μl (1.3 mmol) of 4M hydrogen chloride in dioxane. The mixture was stirred at RT for 16 h. The precipitated product was filtered off with suction, washed with acetonitrile and dried under high vacuum. 53 mg (78% of theory) of the title compound were obtained.

¹H NMR (400 MHz, DMSO-d₆): δ=0.82-1.00 (m, 2H), 1.09-1.33 (m, 2H), 1.46 (m, 2H), 1.51-1.65 (m, 3H), 1.73 (d, 3H), 1.81-1.92 (m, 2H), 1.94-2.03 (m, 2H), 2.03-2.19 (m, 3H), 2.24 (m, 3H), 2.63 (t, 2H), 2.71 (d, 6H), 2.87-2.98 (m, 1H), 3.05-3.23 (m, 2H), 4.46-4.80 (m, 1H), 6.84 (d, 1H), 7.04 (d, 1H), 7.20-7.29 (m, 3H), 7.38 (d, 2H), 7.44 (s, 1H), 7.66-7.78 (m, 2H), 7.85 (br. s., 2H), 8.19-8.26 (m, 1H), 8.28-8.35 (m, 1H), 10.04 (br. s., 1H), 10.30-10.42 (m, 1H), 10.51 (s, 1H), 10.58 (s, 1H).

LC-MS (Method 1): Rₜ=0.57 min; MS (ESIneg): m/z=694 [M−H—HCl]⁻.

### Example 69

### trans-4-(Aminomethyl)-N-[(2S)-3-{4′-[(1,1-dioxidothiomorpholin-4-yl)carbonyl]-2′-methyl-biphenyl-4-yl}-1-oxo-1-{[4-(2H-tetrazol-5-yl)phenyl]amino}propan-2-yl]cyclohexanecarboxamide hydrochloride

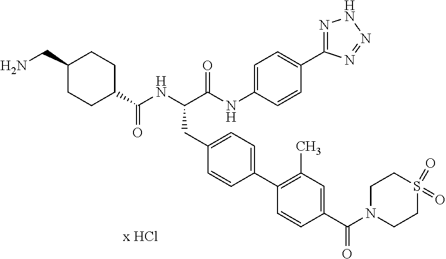

To a solution of 78 mg (0.09 mmol) of tert-butyl [(trans-4-{[(2S)-3-{4′-[(1,1-dioxidothiomorpholin-4-yl)carbonyl]-2′-methylbiphenyl-4-yl}-1-oxo-1-[4-(2H-tetrazol-5-yl)phenyl]amino}-propan-2-yl]carbamoyl}cyclohexyl)methyl]carbamate in 3 ml of dioxane was added 0.32 ml (1.28 mmol) of 4M hydrogen chloride in dioxane. The mixture was stirred at RT for 5 d. The mixture was separated by means of preparative HPLC (eluent: acetonitrile/water gradient, 0.01% trifluoroacetic acid). The product-containing fractions were combined and admixed with a little 4M hydrogen chloride in dioxane and concentrated on a rotary evaporator. 34 mg (50% of theory) of the title compound were obtained.

¹H NMR (400 MHz, DMSO-d₆): δ=0.84-0.99 (m, 2H), 1.11-1.33 (m, 2H), 1.41-1.51 (m, 1H), 1.53-1.63 (m, 1H), 1.69-1.84 (m, 3H), 2.15 (br. s., 1H), 2.21 (s, 3H), 2.64 (t, 2H), 2.97 (t, 1H), 3.10-3.20 (m, 1H), 3.71-4.10 (m, 8H), 4.76 (m, 1H), 7.23 (d, 1H), 7.28 (d, 2H), 7.34-7.44 (m, 4H), 7.75-7.87 (m, 4H), 8.02 (d, 2H), 8.30 (d, 1H), 10.55 (br. s., 1H).

LC-MS (Method 1): Rₜ=0.64 min; MS (ESIneg): m/z=699 [M−H—HCl]⁻.

### Example 70

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-[4-(dimethylamino)cyclohexyl]-2-methylbiphenyl-4-carboxamide hydrochloride

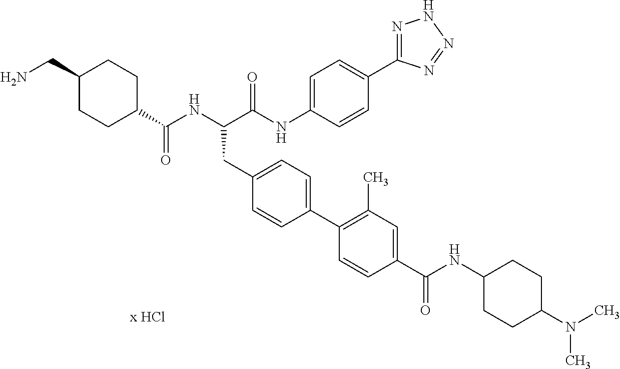

To a solution of 70.8 mg (0.08 mmol) of tert-butyl [(trans-4-{[(2S)-3-(4′-{[4-(dimethylamino)cyclohexyl]carbamoyl}-2′-methylbiphenyl-4-yl)-1-oxo-1-{[4-(2H-tetrazol-5-yl)phenyl]amino}propan-2-yl]carbamoyl}cyclohexyl)methyl]carbamate trifluoroacetate in 3.2 ml of dioxane was added 0.3 ml (1.15 mmol) of 4M hydrogen chloride in dioxane. The mixture was stirred at RT for 16 h. The precipitated product was filtered off with suction, washed with dioxane and dried under high vacuum. 50 mg (78% of theory) of the title compound were obtained.

¹H NMR (400 MHz, DMSO-d₆): δ=0.85-1.00 (m, 2H), 1.11-1.34 (m, 2H), 1.36-1.51 (m, 3H), 1.53-1.66 (m, 4H), 1.69-1.82 (m, 4H), 1.82-1.90 (m, 1H), 1.93-2.03 (m, 2H), 2.04-2.11 (m, 1H), 2.11-2.20 (m, 1H), 2.23 (d, 3H), 2.59-2.67 (m, 2H), 2.71 (br. s., 3H), 2.72 (br. s., 3H), 2.91-3.01 (m, 1H), 3.08-3.22 (m, 2H), 4.70-4.80 (m, 1H), 7.26 (m, 3H), 7.39 (d, 2H), 7.65-7.77 (m, 2H), 7.83 (m, 5H), 8.02 (d, 2H), 8.26-8.36 (m, 2H), 10.19-10.35 (m, 1H), 10.57 (s, 1H).

LC-MS (Method 1): Rₜ=0.63 min; MS (ESIneg): m/z=706 [M−H—HCl]⁻.

### Example 71

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methoxy-N-[(3R)-pyrrolidin-3-yl]biphenyl-4-carboxamide hydrochloride

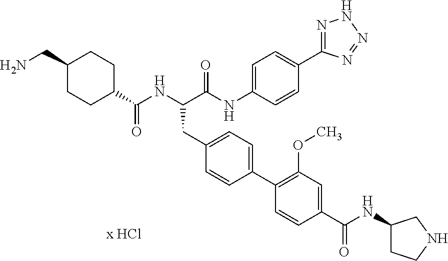

To a solution of 60 mg (0.03 mmol) of tert-butyl (3R)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methoxybiphenyl-4-yl}carbonyl)amino]pyrrolidine-1-carboxylate in 2.5 ml of dioxane was added 0.23 ml (0.92 mmol) of 4M hydrogen chloride in dioxane. The mixture was stirred at RT for 7 d. Another 1.5 ml of 4M hydrogen chloride in dioxane were added and the mixture was stirred at 50° C. for 24 h. The reaction mixture was concentrated and the residue was separated by means of preparative HPLC (eluent: acetonitrile/water gradient with 0.01% trifluoroacetic acid). The product-containing fractions were combined and admixed with a little 4M hydrogen chloride in dioxane and concentrated on a rotary evaporator. The residue was dried under high vacuum. 26 mg (57% of theory) of the title compound were obtained.

¹H NMR (400 MHz, DMSO-d₆): δ=0.87-1.00 (m, 2H), 1.16-1.33 (m, 2H), 1.42-1.52 (m, 1H), 1.59-1.67 (m, 1H), 1.71-1.82 (m, 3H), 1.98-2.08 (m, 1H), 2.12-2.29 (m, 2H), 2.66 (br. s., 2H), 2.88-2.97 (m, 1H), 3.07-3.13 (m, 1H), 3.14-3.23 (m, 1H), 3.24-3.33 (m, 2H), 3.34-3.41 (m, 2H), 3.43-3.52 (m, 2H), 4.44-4.59 (m, 1H), 4.63-4.80 (m, 1H), 7.33-7.39 (m, 3H), 7.43 (d, 2H), 7.50-7.55 (m, 2H), 7.67 (br. s., 3H), 7.82 (d, 2H), 8.00 (d, 2H), 8.25 (d, 1H), 8.65 (d, 1H), 8.74-8.84 (m, 1H), 8.86-8.94 (m, 1H), 10.47 (s, 1H), 16.33-16.92 (m, 1H).

LC-MS (Method 1): Rₜ=0.69 min; MS (ESIneg): m/z=722 [M−H—HCl]⁻.

### Example 72

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(1,3-dihydroxypropan-2-yl)-2-methylbiphenyl-4-carboxamide hydrochloride

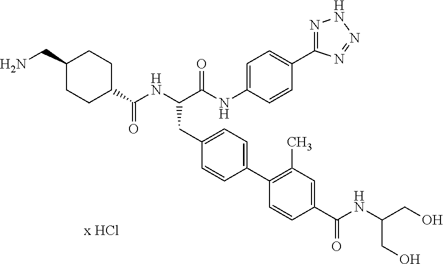

To a solution of 34 mg (0.04 mmol) of tert-butyl [(trans-4-{[(2S)-3-{4′-[(1,3-dihydroxypropan-2-yl)carbamoyl]-2′-methylbiphenyl-4-yl}-1-oxo-1-{[4-(2H-tetrazol-5-yl)phenyl]amino}propan-2-yl]carbamoyl}cyclohexyl)methyl]carbamate in 2 ml of dioxane was added 0.15 ml (0.59 mmol) of 4M hydrogen chloride in dioxane. The mixture was stirred at RT for 16 h. The mixture was concentrated and the residue was separated by means of preparative HPLC (eluent: acetonitrile/water gradient with 0.01% trifluoroacetic acid). The product-containing fractions were combined and admixed with a little 4M hydrogen chloride in dioxane and concentrated on a rotary evaporator. 15 mg (37% of theory, 74% purity) of the title compound were obtained.

¹H NMR (400 MHz, DMSO-d₆): δ=0.81-1.00 (m, 2H), 1.07-1.35 (m, 2H), 1.38-1.51 (m, 1H), 1.54-1.63 (m, 1H), 1.69-1.83 (m, 3H), 2.08-2.20 (m, 1H), 2.23 (s, 3H), 2.59-2.69 (m, 2H), 2.89-3.01 (m, 1H), 3.09-3.20 (m, 1H), 3.52 (d, 3H), 3.92-4.01 (m, 1H), 4.69-4.81 (m, 1H), 7.19-7.35 (m, 3H), 7.36-7.45 (m, 2H), 7.70-7.88 (m, 6H), 7.92-8.06 (m, 3H), 8.23-8.34 (m, 1H), 10.50-10.61 (m, 1H).

LC-MS (Method 1): Rₜ=0.65 min; MS (ESIneg): m/z=655 [M−H—HCl]⁻.

### Example 73

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-methyl-N-[(3S)-1-methylpiperidin-3-yl]biphenyl-4-carboxamide hydrochloride

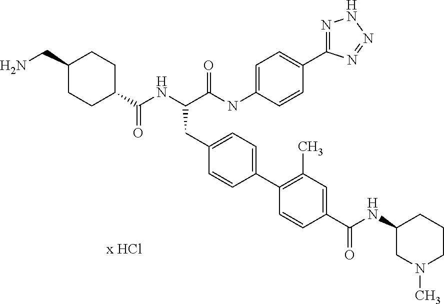

To a solution of 94.2 mg (0.1 mmol) of tert-butyl [(trans-4-{[(2S)-3-{2′-methyl-4′-{[(3S)-1-methylpiperidin-3-yl]carbamoyl}biphenyl-4-yl)-1-oxo-1-{[4-(2H-tetrazol-5-yl)phenyl]amino}-propan-2-yl]carbamoyl}cyclohexyl)methyl]carbamate trifluoroacetate in 4.4 ml of dioxane was added 0.4 ml (1.58 mmol) of 4M hydrogen chloride in dioxane. The mixture was stirred at RT for 16 h. The precipitated product was filtered off with suction, washed with dioxane and dried under high vacuum. 63 mg (80% of theory) of the title compound were obtained.

¹H NMR (400 MHz, DMSO-d₆): δ=0.83-1.02 (m, 2H), 1.23 (s, 3H), 1.41-1.63 (m, 3H), 1.68-1.84 (m, 6H), 1.87-1.97 (m, 2H), 2.11-2.20 (m, 1H), 2.23 (s, 3H), 2.63 (t, 2H), 2.75-2.84 (m, 4H), 2.93-3.04 (m, 1H), 3.10-3.20 (m, 1H), 4.18-4.30 (m, 1H), 4.71-4.80 (m, 1H), 7.26 (m, 3H), 7.41 (d, 2H), 7.72 (d, 1H), 7.78 (s, 1H), 7.84 (m, 5H), 8.03 (d, 2H), 8.28-8.34 (m, 1H), 8.59 (d, 1H), 10.35 (br. s, 1H), 10.58 (s, 1H), 10.93 (br. s, 1H).

LC-MS (Method 1): Rₜ=0.61 min; MS (ESIneg): m/z=678 [M−H—HCl]⁻.

### Example 74

### 4′-[(2S)-2-({[trans-4-(Aminomethyl)cyclohexyl]carbonyl}amino)-3-({4-[3-(difluoromethyl)-1H-1,2,4-triazol-5-yl]phenyl}amino)-3-oxopropyl]-2-methyl-N-(2-oxopiperidin-3-yl)biphenyl-4-carboxamide hydrochloride

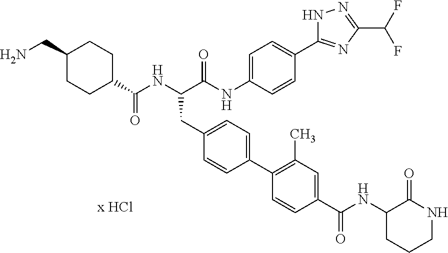

To a solution of 47 mg (0.05 mmol) of tert-butyl [(trans-4-{[(2S)-1-({4-[3-(difluoromethyl)-1H-1,2,4-triazol-5-yl]phenyl}amino)-3-(2′-methyl-4′-{[2-oxopiperidin-3-yl]carbamoyl}biphenyl-4-yl)-1-oxopropan-2-yl]carbamoyl}cyclohexyl)methyl]carbamate in 2 ml of dioxane were added 187 μl (0.75 mmol) of 4M hydrogen chloride in dioxane. The mixture was stirred at RT for 72 h. The precipitated product was filtered off with suction, washed with dioxane and dried under high vacuum. 37 mg (88% of theory) of the title compound were obtained.

¹H NMR (400 MHz, DMSO-d₆): δ=0.93 (d, 2H), 1.11-1.35 (m, 2H), 1.41-1.52 (m, 1H), 1.55-1.61 (m, 1H), 1.68-1.89 (m, 6H), 1.95-2.06 (m, 1H), 2.15 (t, 1H), 2.24 (s, 3H), 2.63 (t, 2H), 2.96 (q, 1H), 3.10-3.23 (m, 3H), 4.28-4.45 (m, 1H), 4.64-4.81 (m, 1H), 7.25 (d, 1H), 7.28 (d, 2H), 7.40 (d, 2H), 7.65 (br. s., 1H), 7.73 (d, 1H), 7.80 (d, 5H), 7.99 (d, 2H), 8.28 (d, 1H), 8.59 (d, 1H), 10.51 (s, 1H), 14.61-15.01 (m, 1H).

LC-MS (Method 1): Rₜ=0.70 min; MS (ESIneg): m/z=727 [M−H—HCl]⁻.

### Example 75

### 4′-[(2S)-2-({[trans-4-(Aminomethyl)cyclohexyl]carbonyl}amino)-3-oxo-3-({4-[3-(pentafluoroethyl)-1H-1,2,4-triazol-5-yl]phenyl}amino)propyl]-2-methyl-N-[(3R)-pyrrolidin-3-yl]biphenyl-4-carboxamide hydrochloride

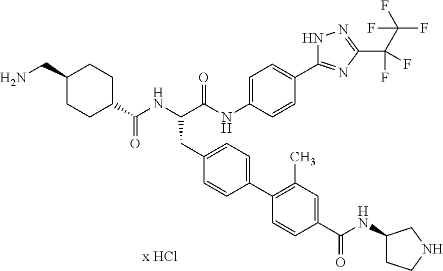

To a solution of 70.2 mg (0.07 mmol) of tert-butyl (3R)-3-[({4′-[(2S)-2-{[(trans-4-{[(tert-butoxycarbonyl)amino]methyl}cyclohexyl)carbonyl]amino}-3-oxo-3-({4-[3-(pentafluoroethyl)-1H-1,2,4-triazol-5-yl]phenyl}amino)propyl]-2-methylbiphenyl-4-yl}carbonyl)amino]pyrrolidine-1-carboxylate in 3 ml of dioxane was added 0.24 ml (0.97 mmol) of 4M hydrogen chloride in dioxane. The mixture was stirred at RT for 48 h. The mixture was concentrated, taken up in 1 ml of dimethylformamide and separated by means of preparative HPLC (eluent: acetonitrile/water gradient with 0.01% trifluoroacetic acid). The product-containing fractions were combined and admixed with a little 4M hydrogen chloride in dioxane and concentrated on a rotary evaporator. The residue was dried under high vacuum. 12 mg (23% of theory) of the title compound were obtained.

¹H NMR (400 MHz, DMSO-d₆): δ=0.91 (m, 2H), 1.12-1.34 (m, 2H), 1.41-1.51 (m, 1H), 1.58 (d, 1H), 1.69-1.83 (m, 3H), 1.96-2.06 (m, 1H), 2.09-2.21 (m, 2H), 2.24 (s, 3H), 2.58-2.69 (m, 2H), 2.97 (m, 1H), 3.10-3.32 (m, 4H), 4.57 (m, 1H), 4.75 (m, 1H), 7.21-7.30 (m, 3H), 7.41 (d, 2H), 7.73-7.88 (m, 7H), 8.02 (d, 2H), 8.29 (d, 1H), 8.74 (m, 1H), 9.12 (br. s, 1H), 9.32 (br. s, 1H), 10.55 (br. s., 1H), 15.40 (br. s., 1H).

LC-MS (Method 2): Rt=1.73 min; MS (ESIneg): m/z=767 [M−H—HCl]⁻.

### Example 76

### 4′-[(2S)-2-({[trans-4-(Aminomethyl)cyclohexyl]carbonyl}amino)-3-oxo-3-{[2-(trifluoromethyl)-1H-benzimidazol-5-yl]amino}propyl]-2-methyl-N-(1-methylpiperidin-4-yl)biphenyl-4-carboxamide hydrochloride

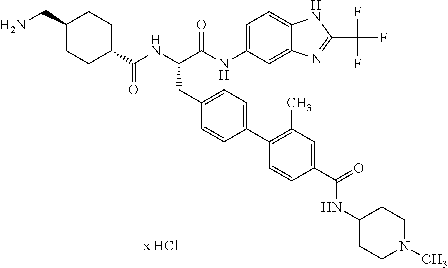 [SMILES: Cc1cc(C(=O)NC2CCN(C)CC2)ccc1-c1ccc(C[C@H](NC(=O)[C@H]2CC[C@H](CN)CC2)C(=O)Nc2ccc3[nH]c(C(F)(F)F)nc3c2)cc1]

A solution of 72.9 mg (0.09 mmol) of tert-butyl [(trans-4-{[(2S)-3-{2′-methyl-4′-[(1-methylpiperidin-4-yl)carbamoyl]biphenyl-4-yl}-1-oxo-1-{[2-(trifluoromethyl)-1H-benzimidazol-5-yl]amino}propan-2-yl]carbamoyl}cyclohexyl)methyl]carbamate in 4 ml of 1,4-dioxane was admixed with 1 ml (4.00 mmol) of 4M hydrogen chloride in 1,4-dioxane and stirred at RT for 3 h. The solvent was removed on a rotary evaporator, and the residue was stirred in acetonitrile and filtered. The solid formed was washed with acetonitrile and dried under high vacuum. 55 mg (78% of theory) of the title compound were obtained.

¹H NMR (400 MHz, DMSO-d₆): δ=0.82-1.00 (m, 2H), 1.06-1.35 (m, 2H), 1.39-1.63 (m, 2H), 1.74 (m, 3H), 1.85-2.04 (m, 4H), 2.11-2.20 (m, 1H), 2.23 (s, 3H), 2.63 (t, 2H), 2.69-2.79 (m, 3H), 2.90-3.19 (m, 4H), 3.25-3.32 (m, 1H), 3.38-3.46 (m, 2H), 3.98-4.08 (m, 1H), 4.77 (m, 1H), 7.13-7.30 (m, 3H), 7.35-7.49 (m, 3H), 7.68 (d, 1H), 7.74 (d, 1H), 7.80 (s, 1H), 7.89 (br. s., 3H), 8.21 (s, 1H), 8.29 (d, 1H), 8.55 (d, 1H), 10.25-10.59 (m, 2H).

LC-MS (Method 1): Rₜ=0.61 min; MS (ESIneg): m/z=716 [M−H—HCl]⁻.

### Example 77

### 4′-[(2S)-2-({[trans-4-(Aminomethyl)cyclohexyl]carbonyl}amino)-3-oxo-3-{[2-(trifluoromethyl)-1H-benzimidazol-5-yl]amino}propyl]-N-[4-(dimethylamino)cyclohexyl]-2-methylbiphenyl-4-carboxamide hydrochloride

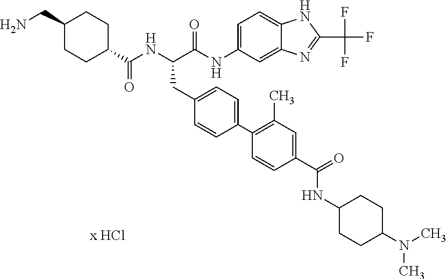 [SMILES: Cc1cc(C(=O)NC2CCC(N(C)C)CC2)ccc1-c1ccc(C[C@H](NC(=O)[C@H]2CC[C@H](CN)CC2)C(=O)Nc2ccc3[nH]c(C(F)(F)F)nc3c2)cc1]

A solution of 64.4 mg (0.08 mmol) of tert-butyl [(trans-4-{[(2S)-3-(4′-{[4-(dimethylamino)cyclohexyl]carbamoyl}-2′-methylbiphenyl-4-yl)-1-oxo-1-{[2-(trifluoromethyl)-1H-benzimidazol-5-yl]amino}propan-2-yl]carbamoyl}cyclohexyl)methyl]carbamate in 4 ml of 1,4-dioxane was admixed with 1 ml (4.00 mmol) of 4M hydrogen chloride in 1,4-dioxane and stirred at RT for 3 h. The solvent was removed on a rotary evaporator, and the residue was stirred in acetonitrile and filtered. The solid formed was washed with acetonitrile and dried under high vacuum. 52 mg (83% of theory) of the title compound were obtained.

¹H NMR (400 MHz, DMSO-d₆): δ=0.83-1.05 (m, 2H), 1.10-1.35 (m, 2H), 1.35-1.89 (m, 9H), 1.90-2.02 (m, 2H), 2.03-2.24 (m, 5H), 2.63 (t, 2H), 2.71 (d, 6H), 2.88-3.03 (m, 1H), 3.15 (m, 2H), 3.59-3.63 (m, 1H), 3.72-3.85 (m, 1H), 4.04-4.15 (m, 1H), 4.77 (m, 1H), 7.16-7.32 (m, 3H), 7.35-7.53 (m, 3H), 7.63-7.78 (m, 3H), 7.89 (br. s., 2H), 8.21 (s, 1H), 8.30 (dd, 1H), 10.42 (m, 2H).

LC-MS (Method 1): Rₜ=0.62 min; MS (ESIneg): m/z=744 [M−H—HCl]⁻.

### Example 78

### 4′-[(2S)-2-({[trans-4-(Aminomethyl)cyclohexyl]carbonyl}amino)-3-oxo-3-{[2-(pentafluoroethyl)-1H-benzimidazol-5-yl]amino}propyl]-N-[4-(dimethylamino)cyclohexyl]-2-methylbiphenyl-4-carboxamide hydrochloride

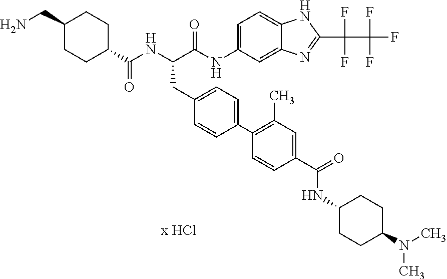 [SMILES: Cc1cc(C(=O)N[C@H]2CC[C@H](N(C)C)CC2)ccc1-c1ccc(C[C@H](NC(=O)[C@H]2CC[C@H](CN)CC2)C(=O)Nc2ccc3[nH]c(C(F)(F)C(F)(F)F)nc3c2)cc1]

A solution of 101.5 mg (0.11 mmol) of tert-butyl [(trans-4-{[(2S)-3-(4′-{[4-(dimethylamino)cyclohexyl]carbamoyl}-2′-methylbiphenyl-4-yl)-1-oxo-1-{[2-(pentafluoroethyl)-1H-benzimidazol-5-yl]amino}propan-2-yl]carbamoyl}cyclohexyl)methyl]carbamate in 8 ml of 1,4-dioxane was admixed with 2 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 99 mg (91% of theory, 90% purity) of the title compound were obtained.

¹H NMR (400 MHz, DMSO-d₆): δ=0.80-1.01 (m, 2H), 1.10-1.33 (m, 2H), 1.35-1.66 (m, 5H), 1.74 (d, 5H), 2.23 (m, 7H), 2.58-2.77 (m, 9H), 2.86-3.03 (m, 1H), 3.05-3.27 (m, 2H), 4.03-4.18 (m, 1H), 4.66-4.90 (m, 1H), 7.25 (m, 3H), 7.40 (m, 3H), 7.71 (m, 3H), 7.88 (br. s., 3H), 8.23 (s, 3H), 10.45 (s, 2H).

LC-MS (Method 1): Rₜ=0.65 min; MS (ESIneg): m/z=794 [M−H—HCl]⁻.

### Example 79

### N-(trans-4-Aminocyclohexyl)-4′-[(2S)-2-({[trans-4-(aminomethyl)cyclohexyl]carbonyl}amino)-3-oxo-3-{[2-(pentafluoroethyl)-1H-benzimidazol-5-yl]amino}propyl]-2-methylbiphenyl-4-carboxamide hydrochloride

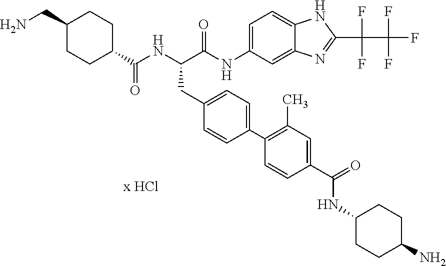 [SMILES: Cc1cc(C(=O)N[C@H]2CC[C@H](N)CC2)ccc1-c1ccc(C[C@H](NC(=O)[C@H]2CC[C@H](CN)CC2)C(=O)Nc2ccc3[nH]c(C(F)(F)C(F)(F)F)nc3c2)cc1]

A solution of 110.5 mg (0.11 mmol) of tert-butyl [(trans-4-{[(2S)-3-[4′-({trans-4-{[(tert-butoxycarbonyl)amino]cyclohexyl}carbamoyl)-2′-methylbiphenyl-4-yl]-1-oxo-1-{[2-(pentafluoroethyl)-1H-benzimidazol-5-yl]amino}propan-2-yl]carbamoyl}cyclohexyl)methyl]-carbamate in 2 ml of 1,4-dioxane was admixed with 1 ml (4.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 17 mg (16% of theory) of the title compound were obtained.

¹H NMR (400 MHz, DMSO-d₆): δ=0.82-1.00 (m, 2H), 1.11-1.51 (m, 10H), 1.66-1.82 (m, 3H), 1.84-2.04 (m, 4H), 2.21 (s, 4H), 2.62-2.71 (m, 2H), 2.90-3.06 (m, 2H), 3.07-3.18 (m, 1H), 4.63-4.88 (m, 1H), 7.17-7.46 (m, 5H), 7.57-7.93 (m, 10H), 8.13-8.32 (m, 3H), 10.31 (s, 1H), 13.90 (br. s, 1H).

LC-MS (Method 1): Rₜ=0.68 min; MS (ESIneg): m/z=766 [M−H—HCl]⁻.

### Example 80

### 4′-[(2S)-2-({[trans-4-(Aminomethyl)cyclohexyl]carbonyl}amino)-3-oxo-3-{[2-(pentafluoroethyl)-1H-benzimidazol-5-yl]amino}propyl]-2-methyl-N-[(3R)-pyrrolidin-3-yl]biphenyl-4-carboxamide hydrochloride

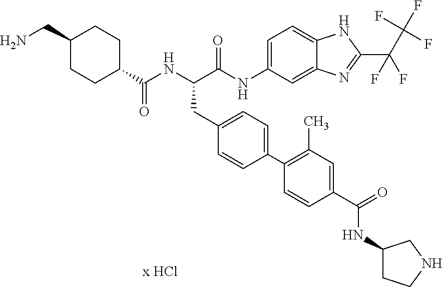 [SMILES: Cc1cc(C(=O)N[C@@H]2CCNC2)ccc1-c1ccc(C[C@H](NC(=O)[C@H]2CC[C@H](CN)CC2)C(=O)Nc2ccc3[nH]c(C(F)(F)C(F)(F)F)nc3c2)cc1]

A solution of 96.3 mg (0.11 mmol) of tert-butyl (3R)-3-[({4′-[(2S)-2-{[(trans-4-{[(tert-butoxycarbonyl)amino]methyl}cyclohexyl)carbonyl]amino}-3-oxo-3-{[2-(pentafluoroethyl)-1H-benzimidazol-5-yl]amino}propyl]-2-methylbiphenyl-4-yl}carbonyl)amino]pyrrolidine-1-carboxylate in 2 ml of 1,4-dioxane was admixed with 2 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and separated by means of preparative HPLC (eluent: methanol/water gradient, 0.01% trifluoroacetic acid). The product-containing fractions were combined, admixed with 2 ml of a 1M hydrochloride solution and concentrated on a rotary evaporator, and the residue was dried under high vacuum. 8 mg (8% of theory) of the title compound were obtained.

¹H NMR (400 MHz, DMSO-d₆): δ=0.79-1.02 (m, 2H), 1.11-1.34 (m, 2H), 1.41-1.63 (m, 2H), 1.66-1.83 (m, 3H), 1.97-2.08 (m, 1H), 2.11-2.32 (m, 5H), 2.59-2.70 (m, 2H), 2.88-3.03 (m, 1H), 3.07-3.29 (m, 2H), 3.31-3.48 (m, 2H), 4.47-4.61 (m, 1H), 4.68-4.86 (m, 1H), 7.18-7.34 (m, 3H), 7.36-7.50 (m, 3H), 7.68-7.90 (m, 6H), 8.16-8.31 (m, 2H), 8.73 (d, 1H), 9.01 (br. s, 1H), 9.24 (br. s, 1H), 10.38 (s, 1H).

LC-MS (Method 1): Rₜ=0.69 min; MS (ESIneg): m/z=738 [M−H—HCl]⁻.

### Example 81

### 4′-[(2S)-2-({[trans-4-(Aminomethyl)cyclohexyl]carbonyl}amino)-3-oxo-3-{[2-(pentafluoroethyl)-1H-benzimidazol-5-yl]amino}propyl]-N-[3-(dimethylamino)propyl]-2-methylbiphenyl-4-carboxamide hydrochloride

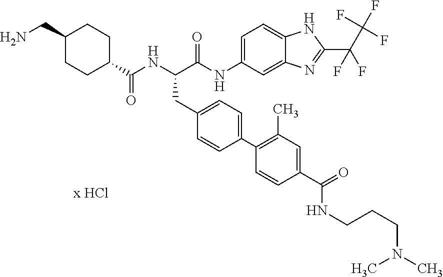 [SMILES: Cc1cc(C(=O)NCCCN(C)C)ccc1-c1ccc(C[C@H](NC(=O)[C@H]2CC[C@H](CN)CC2)C(=O)Nc2ccc3[nH]c(C(F)(F)C(F)(F)F)nc3c2)cc1]

A solution of 42 mg (0.05 mmol) of tert-butyl [(trans-4-{[(2S)-3-(4′-{[3-(dimethylamino)propyl]-carbamoyl}-2′-methylbiphenyl-4-yl)-1-oxo-1-{[2-(pentafluoroethyl)-1H-benzimidazol-5-yl]amino}-propan-2-yl]carbamoyl}cyclohexyl)methyl]carbamate in 1 ml of 1,4-dioxane was admixed with 0.25 ml (2.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 34 mg (79% of theory) of the title compound were obtained.

¹H NMR (400 MHz, DMSO-d₆): δ=0.78-1.00 (m, 2H), 1.09-1.35 (m, 2H), 1.41-1.65 (m, 2H), 1.68-1.83 (m, 3H), 1.87-1.98 (m, 2H), 2.10-2.25 (m, 4H), 2.63 (t, 2H), 2.75 (d, 6H), 2.91-3.02 (m, 1H), 3.03-3.18 (m, 3H), 3.35 (q, 2H), 4.77 (d, 1H), 7.17-7.31 (m, 3H), 7.33-7.54 (m, 3H), 7.63-7.98 (m, 6H), 8.16-8.42 (m, 2H), 8.70 (t, 1H), 10.19 (br. s, 1H), 10.44 (s, 1H).

LC-MS (Method 1): Rₜ=0.66 min; MS (ESIneg): m/z=754 [M−H—HCl]⁻.

### Example 82

### 4′-[(2S)-2-({[trans-4-(Aminomethyl)cyclohexyl]carbonyl}amino)-3-oxo-3-{[2-(pentafluoroethyl)-1H-benzimidazol-5-yl]amino}propyl]-2-methyl-N-(1-methylpiperidin-4-yl)biphenyl-4-carboxamide hydrochloride

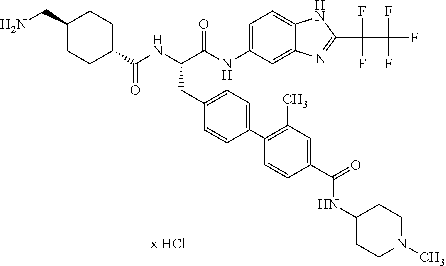 [SMILES: Cc1cc(C(=O)NC2CCN(C)CC2)ccc1-c1ccc(C[C@H](NC(=O)[C@H]2CC[C@H](CN)CC2)C(=O)Nc2ccc3[nH]c(C(F)(F)C(F)(F)F)nc3c2)cc1]

A solution of 22 mg (0.03 mmol) of tert-butyl [(trans-4-{[(2S)-3-{2′-methyl-4′-[(1-methylpiperidin-4-yl)carbamoyl]biphenyl-4-yl}-1-oxo-1-{[2-(pentafluoroethyl)-1H-benzimidazol-5-yl]amino}propan-2-yl]carbamoyl}cyclohexyl)methyl]carbamate in 2 ml of 1,4-dioxane was admixed with 0.5 ml (2.00 mmol) of 4M hydrogen chloride in 1,4-dioxane and stirred at RT for 3 h. The solvent was removed on a rotary evaporator, and the residue was stirred in acetonitrile and filtered. The solid formed was washed with acetonitrile and dried under high vacuum. 20 mg (93% of theory) of the title compound were obtained.

¹H NMR (400 MHz, DMSO-d₆): δ=0.83-1.02 (m, 2H), 1.11-1.35 (m, 2H), 1.41-1.64 (m, 2H), 1.68-2.05 (m, 7H), 2.12-2.25 (m, 4H), 2.64 (br. s., 2H), 2.73 (m, 3H), 2.91-3.19 (m, 4H), 3.36-3.48 (m, 3H), 3.95-4.09 (m, 1H), 4.71-4.84 (m, 1H), 7.18-7.29 (m, 3H), 7.34-7.51 (m, 1H), 7.63-7.87 (m, 6H), 8.17-8.29 (m, 2H), 8.41-8.57 (m, 1H), 10.11-10.44 (m, 2H), 13.83 (br. s, 1H).

LC-MS (Method 1): Rₜ=0.65 min; MS (ESIneg): m/z=766 [M−H—HCl]⁻.

### Example 83

### 4′-[(2S)-2-({[trans-4-(Aminomethyl)cyclohexyl]carbonyl}amino)-3-{[4-(3-chloro-4H-1,2,4-triazol-5-yl)phenyl]amino}-3-oxopropyl]-2-methyl-N-[(3S)-pyrrolidin-3-yl]biphenyl-4-carboxamide hydrochloride

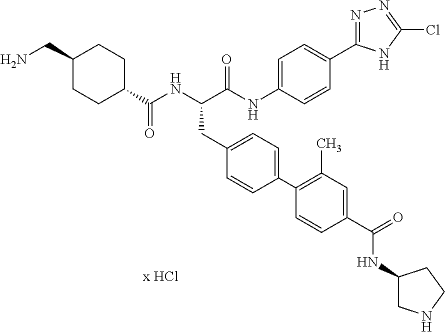 [SMILES: Cc1cc(C(=O)N[C@H]2CCNC2)ccc1-c1ccc(C[C@H](NC(=O)[C@H]2CC[C@H](CN)CC2)C(=O)Nc2ccc(-c3nnc(Cl)[nH]3)cc2)cc1]

A solution of 48 mg (0.05 mmol) of tert-butyl (3S)-3-[({4′-[(2S)-2-{[(trans-4-{[(tert-butoxycarbonyl)amino]methyl}cyclohexyl)carbonyl]amino}-3-[4-(3-chloro-4H-1,2,4-triazol-5-yl)phenyl]amino}-3-oxopropyl]-2-methylbiphenyl-4-yl}carbonyl)amino]pyrrolidine-1-carboxylate in 2 ml of 1,4-dioxane was admixed with 1 ml (4.00 mmol) of 4M hydrogen chloride in 1,4-dioxane and stirred at RT for 16 h. The mixture was separated by means of preparative HPLC (eluent: methanol/water gradient, 0.01% trifluoroacetic acid). The product-containing fractions were combined, admixed with 5 ml of a 1M hydrochloride solution and concentrated on a rotary evaporator, and the residue was dried under high vacuum. 14 mg (35% of theory) of the title compound were obtained.

¹H NMR (400 MHz, DMSO-d₆): δ=0.82-1.00 (m, 2H), 1.08-1.34 (m, 2H), 1.39-1.61 (m, 2H), 1.68-1.85 (m, 3H), 1.93-2.06 (m, 1H), 2.10-2.20 (m, 2H), 2.23 (s, 3H), 2.63 (br. s., 2H), 2.90-3.02 (m, 1H), 3.10-3.29 (m, 3H), 3.37-3.42 (m, 2H), 4.49-4.61 (m, 1H), 4.65-4.83 (m, 1H), 7.22-7.29 (m, 2H), 7.40 (d, 2H), 7.78 (d, 2H), 7.85 (br. s., 3H), 7.93 (d, 2H), 8.25-8.33 (m, 1H), 8.53 (br. s, 3H), 8.72-8.84 (m, 1H), 9.15 (br. s, 1H), 9.39 (br. s, 1H), 10.53 (s, 1H), 14.79 (br. s, 1H).

LC-MS (Method 1): Rₜ=0.58 min; MS (ESIneg): m/z=681 [M−H—HCl]⁻.

### Example 84

### 4′-[(2S)-2-({[trans-4-(Aminomethyl)cyclohexyl]carbonyl}amino)-3-{[4-(1H-imidazol-4-yl)phenyl]amino}-3-oxopropyl]-2-methyl-N-(piperidin-4-yl)biphenyl-4-carboxamide hydrochloride

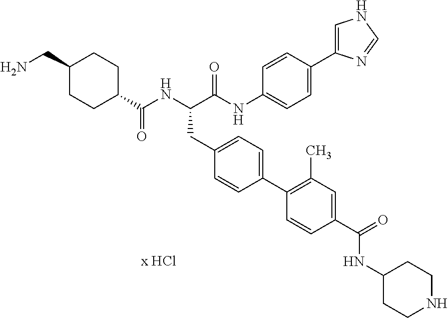 [SMILES: Cc1cc(C(=O)NC2CCNCC2)ccc1-c1ccc(C[C@H](NC(=O)[C@H]2CC[C@H](CN)CC2)C(=O)Nc2ccc(-c3c[nH]cn3)cc2)cc1]

A solution of 134 mg (0.16 mmol) of tert-butyl 4-[({4′-[(2S)-2-{[(trans-4-{[(tert-butoxycarbonyl)amino]methyl}cyclohexyl)carbonyl]amino}-3-{[4-(1H-imidazol-4-yl)phenyl]-amino}-3-oxopropyl]-2-methylbiphenyl-4-yl}carbonyl)amino]piperidine-1-carboxylate in 6 ml of tetrahydrofuran was admixed with 1.25 ml (5 mmol) of 4M hydrogen chloride in 1,4-dioxane and stirred at RT for 16 h. The solid formed was washed with acetonitrile and dried under high vacuum. 93 mg (73% of theory, 90% purity) of the title compound were obtained.

¹H NMR (400 MHz, DMSO-d₆): δ=0.81-1.00 (m, 2H), 1.12-1.34 (m, 2H), 1.43-1.60 (m, 2H), 1.69-1.89 (m, 5H), 1.96 (m, 2H), 2.15 (t, 1H), 2.24 (s, 3H), 2.61 (m, 2H), 2.89-3.17 (m, 4H), 3.25-3.34 (m, 2H), 3.95-4.19 (m, 1H), 4.65-4.84 (m, 1H), 7.13-7.33 (m, 3H), 7.41 (d, 2H), 7.70-8.10 (m, 11H), 8.20-8.40 (m, 1H), 8.53 (d, 1H), 8.85-9.26 (m, 3H), 10.56 (br. s., 1H), 14.82 (br. s, 1H).

LC-MS (Method 1): Rₜ=0.50 min; MS (ESIneg): m/z=660 [M−H—HCl]⁻.

### Example 85

### 4′-[(2S)-2-({[trans-4-(Aminomethyl)cyclohexyl]carbonyl}amino)-3-{[2-(heptafluoropropyl)-1H-benzimidazol-6-yl]amino}-3-oxopropyl]-2-methyl-N-(piperidin-4-yl)biphenyl-4-carboxamide hydrochloride

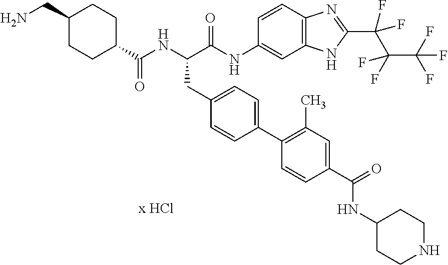 [SMILES: Cc1cc(C(=O)NC2CCNCC2)ccc1-c1ccc(C[C@H](NC(=O)[C@H]2CC[C@H](CN)CC2)C(=O)Nc2ccc3nc(C(F)(F)C(F)(F)C(F)(F)F)[nH]c3c2)cc1]

A solution of 62 mg (0.06 mmol) of tert-butyl 4-[({4′-[(2S)-2-{[(trans-4-{[(tert-butoxycarbonyl)amino]methyl}cyclohexyl)carbonyl]amino}-3-{[2-(heptafluoropropyl)-1H-benzimidazol-6-yl]amino}-3-oxopropyl]-2-methylbiphenyl-4-yl}carbonyl)amino]piperidine-1-carboxylate in 2 ml of dioxane was admixed with 0.8 ml (3 mmol) of 4M hydrogen chloride in 1,4-dioxane and stirred at RT for 16 h and the solvent was removed. The solid formed was washed with acetonitrile and dried under high vacuum. 49 mg (91% of theory) of the title compound were obtained.

¹H NMR (400 MHz, DMSO-d₆): δ=0.82-0.99 (m, 2H), 1.09-1.35 (m, 2H), 1.58 (m, 2H), 1.68-1.88 (m, 5H), 1.97 (m, 2H), 2.10-2.26 (m, 4H), 2.63 (t, 2H), 2.90-3.06 (m, 3H), 3.09-3.19 (m, 1H), 3.31 (m, 2H), 4.01-4.13 (m, 1H), 4.78 (m, 1H), 7.17-7.31 (m, 3H), 7.41 (d, 2H), 7.47 (d, 1H), 7.73 (dd, 2H), 7.79 (s, 1H), 7.93 (br. s., 3H), 8.24 (d, 1H), 8.30 (d, 1H), 8.52 (d, 1H), 8.96 (br. s., 2H), 10.47 (br. s., 1H).

LC-MS (Method 1): Rₜ=0.66 min; MS (ESIneg): m/z=802 [M−H—HCl]⁻.

### Example 86

### 4′-[(2S)-2-({[trans-4-(Aminomethyl)cyclohexyl]carbonyl}amino)-3-{[2-(difluoromethyl)-1H-benzimidazol-6-yl]amino}-3-oxopropyl]-2-methyl-N-(piperidin-4-yl)biphenyl-4-carboxamide hydrochloride

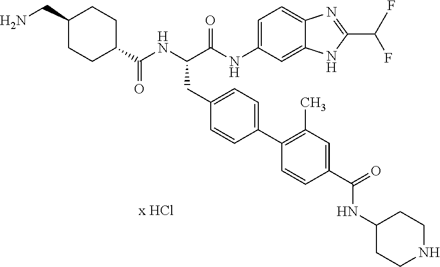 [SMILES: Cc1cc(C(=O)NC2CCNCC2)ccc1-c1ccc(C[C@H](NC(=O)[C@H]2CC[C@H](CN)CC2)C(=O)Nc2ccc3nc(C(F)F)[nH]c3c2)cc1]

A solution of 127 mg (0.14 mmol) of tert-butyl 4-[({4′-[(2S)-2-{[(trans-4-{[(tert-butoxycarbonyl)amino]methyl}cyclohexyl)carbonyl]amino}-3-{[2-(difluoromethyl)-1H-benzimidazol-6-yl]amino}-3-oxopropyl]-2-methylbiphenyl-4-yl}carbonyl)amino]piperidine-1-carboxylate in 3 ml of 1,4-dioxane was admixed with 0.5 ml (2 mmol) of 4M hydrogen chloride in 1,4-dioxane and stirred at RT for 3 h. The solid formed was filtered off, washed with dioxane and dried under high vacuum. 107 mg (98% of theory) of the title compound were obtained.

¹H NMR (400 MHz, DMSO-d₆): δ=0.84-1.00 (m, 2H), 1.08-1.34 (m, 2H), 1.41-1.62 (m, 2H), 1.78 (m, 5H), 1.91-2.02 (m, 2H), 2.11-2.20 (m, 1H), 2.23 (s, 3H), 2.58-2.68 (m, 2H), 2.93-3.05 (m, 3H), 3.09-3.19 (m, 1H), 3.24-3.35 (m, 2H), 3.99-4.15 (m, 1H), 4.66-4.84 (m, 1H), 7.19-7.28 (m, 4H), 7.35-7.44 (m, 3H), 7.61 (d, 1H), 7.73 (d, 1H), 7.79 (s, 1H), 7.86 (br. s., 3H), 8.14 (s, 1H), 8.27 (d, 1H), 8.51 (d, 1H), 8.80 (br. s, 2H), 10.25-10.37 (m, 1H).

LC-MS (Method 1): Rₜ=0.57 min; MS (ESIneg): m/z=685 [M−H—HCl]⁻.

### Example 87

### 4′-[(2S)-2-({[trans-4-(Aminomethyl)cyclohexyl]carbonyl}amino)-3-({4-[3-(heptafluoropropyl)-1H-1,2,4-triazol-5-yl]phenyl}amino)-3-oxopropyl]-N-[4-(dimethylamino)cyclohexyl]-2-methylbiphenyl-4-carboxamide hydrochloride

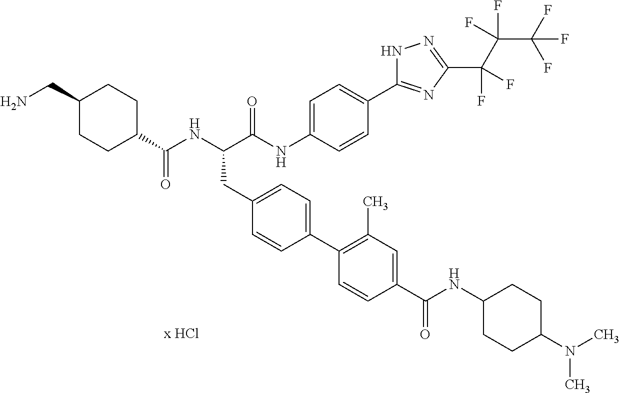

A solution of 75 mg (0.07 mmol) of tert-butyl [(trans-4-{[(2S)-3-(4′-{[4-(dimethylamino)-cyclohexyl]carbamoyl}-2′-methylbiphenyl-4-yl)-1-({4-[3-(heptafluoropropyl)-1H-1,2,4-triazol-5-yl]phenyl}amino)-1-oxopropan-2-yl]carbamoyl}cyclohexyl)methyl]carbamate trifluoroacetate in 3 ml of 1,4-dioxane was admixed with 0.2 ml (1 mmol) of 4M hydrogen chloride in 1,4-dioxane and stirred at RT for 5 d. The solid formed was filtered off, washed with dioxane and dried under high vacuum. 54 mg (83% of theory) of the title compound were obtained.

¹H NMR (400 MHz, DMSO-d₆): δ=0.85-1.02 (m, 2H), 1.25 (d, 2H), 1.38-1.67 (m, 5H), 1.69-1.89 (m, 4H), 1.92-2.02 (m, 2H), 2.05-2.19 (m, 2H), 2.24 (s, 3H), 2.63 (t, 2H), 2.71 (d, 6H), 2.98 (m, 1H), 3.14 (m, 2H), 3.72-3.88 (m, 1H), 4.03-4.13 (m, 1H), 4.75 (m, 1H), 7.20-7.29 (m, 3H), 7.37-7.44 (m, 2H), 7.67-7.77 (m, 2H), 7.79-7.90 (m, 5H), 8.02 (d, 2H), 8.31 (t, 2H), 10.25-10.43 (m, 1H), 10.57 (br. s., 1H), 15.46 (br. s., 1H).

LC-MS (Method 1): Rₜ=0.76 min; MS (ESIneg): m/z=871 [M−H—HCl]⁻.

### Example 88

### 4′-[(2S)-2-({[trans-4-(Aminomethyl)cyclohexyl]carbonyl}amino)-3-({4-[3-(heptafluoropropyl)-1H-1,2,4-triazol-5-yl]phenyl}amino)-3-oxopropyl]-2-methyl-N-(2-oxopiperidin-3-yl)biphenyl-4-carboxamide hydrochloride

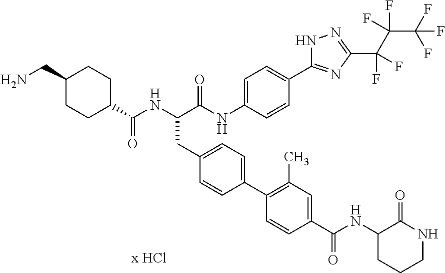

A solution of 50 mg (0.05 mmol) of tert-butyl [(trans-4-{[(2S)-1-({4-[3-(heptafluoropropyl)-1H-1,2,4-triazol-5-yl]phenyl}amino)-3-{2′-methyl-4′-[(2-oxopiperidin-3-yl)carbamoyl]biphenyl-4-yl}-1-oxopropan-2-yl]carbamoyl}cyclohexyl)methyl]carbamate in 2 ml of 1,4-dioxane was admixed with 0.2 ml (1 mmol) of 4M hydrogen chloride in 1,4-dioxane and stirred at RT for 5 d. The solid formed was filtered off, washed with dioxane and dried under high vacuum. 43 mg (98% of theory) of the title compound were obtained.

¹H NMR (400 MHz, DMSO-d₆): δ=0.93 (m, 2H), 1.09-1.33 (m, 2H), 1.41-1.62 (m, 2H), 1.68-1.89 (m, 6H), 1.95-2.05 (m, 1H), 2.15 (t, 1H), 2.24 (s, 3H), 2.64 (m, 2H), 2.90-3.01 (m, 1H), 3.08-3.29 (m, 3H), 4.34-4.42 (m, 1H), 4.64-4.83 (m, 1H), 7.17-7.30 (m, 3H), 7.40 (d, 2H), 7.65 (s, 1H), 7.70-7.85 (m, 7H), 8.02 (d, 2H), 8.27 (d, 1H), 8.58 (d, 1H), 10.54 (s, 1H), 15.40 (br. s, 1H).

LC-MS (Method 1): Rₜ=0.86 min; MS (ESIneg): m/z=844 [M−H—HCl]⁻.

### Example 89

### 4′-[(2S)-2-({[trans-4-(Aminomethyl)cyclohexyl]carbonyl}amino)-3-{[3-fluoro-4-(2H-tetrazol-5-yl)phenyl]amino}-3-oxopropyl]-N-(1-isopropylpiperidin-4-yl)-2-methylbiphenyl-4-carboxamide hydrochloride

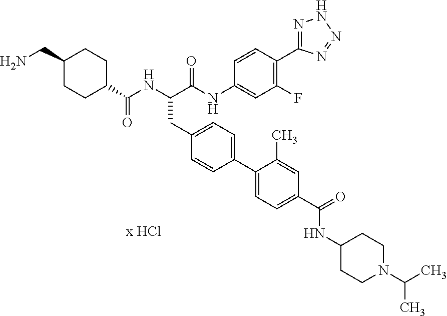

A suspension of 45 mg (0.082 mmol) of tert-butyl [(trans-4-{[(2S)-1-{[3-fluoro-4-(2H-tetrazol-5-yl)phenyl]amino}-3-{4′-[(1-isopropylpiperidin-4-yl)carbamoyl]-2′-methylbiphenyl-4-yl}-1-oxopropan-2-yl]carbamoyl}cyclohexyl)methyl]carbamate in 3.3 ml of dichloromethane was admixed with 0.14 ml (0.54 mmol) of 4M hydrogen chloride in dioxane and stirred at 35° C. overnight. Subsequently, the mixture was admixed with acetonitrile, and the residue was filtered off and dried under reduced pressure. This gave 34 mg (77% of theory) of the title compound.

¹H NMR (300 MHz, DMSO-d₆): δ=0.82-1.00 (m, 2H), 1.13-1.22 (m, 1H), 1.26 (br. s., 3H), 1.28 (br. s., 3H), 1.39-1.53 (m, 1H), 1.53-1.63 (m, 1H), 1.67-1.84 (m, 4H), 1.92-2.04 (m, 4H), 2.08-2.17 (m, 1H), 2.20 (s, 3H), 2.59-2.68 (m, 3H), 2.87-3.03 (m, 2H), 3.04-3.15 (m, 3H), 3.42-3.48 (m, 2H), 3.99-4.12 (m, 1H), 4.64-4.78 (m, 1H), 7.19-7.29 (m, 3H), 7.39 (d, 2H), 7.50-7.56 (m, 1H), 7.68-7.89 (m, 8H), 7.96-8.07 (m, 1H), 8.28-8.35 (m, 1H), 8.50-8.57 (m, 1H), 9.81-9.93 (m, 1H), 10.76 (s, 1H)

LC-MS (Method 4): Rₜ=0.71 min; MS (ESIpos): m/z=724.4 [M+H—HCl]⁺.

### Example 90

### 4′-[(2S)-2-({[trans-4-(Aminomethyl)cyclohexyl]carbonyl}amino)-3-{[3-fluoro-4-(2H-tetrazol-5-yl)phenyl]amino}-3-oxopropyl]-N-(5,5-difluoropiperidin-3-yl)-2-methylbiphenyl-4-carboxamide hydrochloride

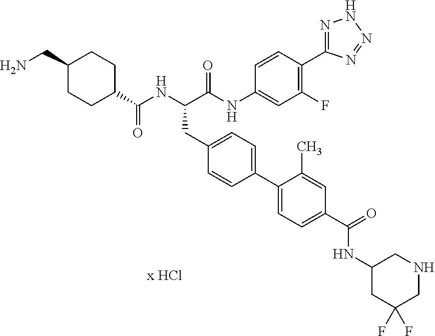

A suspension of 29 mg (0.032 mmol) of tert-butyl [(trans-4-{[(2S)-1-{[3-fluoro-4-(2H-tetrazol-5-yl)phenyl]amino}-3-4′-[(1-isopropylpiperidin-4-yl)carbamoyl-2′-methylbiphenyl-4-yl}-1-oxopropan-2-yl]carbamoyl}cyclohexyl)methyl]carbamate in 2.0 ml of dichloromethane was admixed with 0.08 ml (0.32 mmol) of 4M hydrogen chloride in dioxane and stirred at 35° C. overnight. Subsequently, the mixture was admixed with acetonitrile, and the residue was filtered off and dried under reduced pressure. This gave 14 mg (52% of theory) of the title compound.

¹H NMR (300 MHz, DMSO-d₆): δ=0.79-1.01 (m, 2H), 1.13-1.30 (m, 3H), 1.39-1.52 (m, 1H), 1.52-1.62 (m, 1H), 1.67-1.82 (m, 3H), 2.09-2.19 (m, 1H), 2.21 (s, 3H), 2.57-2.67 (m, 2H), 2.95-3.07 (m, 2H), 3.08-3.17 (m, 2H), 3.52-3.61 (m, 1H), 3.62-3.76 (m, 2H), 4.31-4.51 (m, 1H), 4.66-4.78 (m, 1H), 7.26 (m, 3H), 7.38 (d, 2H), 7.51-7.58 (m, 1H), 7.71-7.77 (m, 1H), 7.82 (m, 5H), 8.02 (t, 1H), 8.29-8.37 (m, 1H), 8.78 (d, 1H), 10.82 (s, 1H).

LC-MS (Method 4): Rₜ=0.70 min; MS (ESIpos): m/z=718.4 [M+H—HCl]⁺.

### Example 91

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-[2-(pyrrolidin-1-yl)ethyl]biphenyl-4-carboxamide hydrochloride

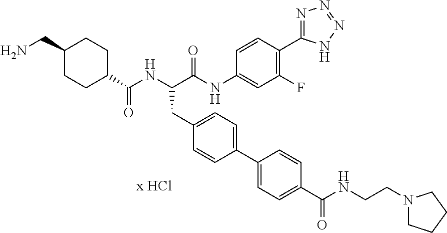

A suspension of 61 mg (0.08 mmol) of tert-butyl [(trans-4-{[(2S)-1-oxo-3-(4′-{[2-(pyrrolidin-1-yl)ethyl]carbamoyl}biphenyl-4-yl)-1-{[4-(1H-tetrazol-5-yl)phenyl]amino}propan-2-yl]carbamoyl}cyclohexyl)methyl]carbamate in 3.4 ml of dichloromethane was admixed with 0.2 ml (0.8 mmol) of 4M hydrogen chloride in dioxane and stirred at 35° C. overnight. Subsequently, acetonitrile was added. The residue was filtered off, dried under reduced pressure and purified by chromatography via HPLC (Method 7). This gave 27 mg (45% of theory) of the title compound.

¹H NMR (300 MHz, DMSO-d₆): δ=0.77-0.97 (m, 2H), 1.09-1.34 (m, 2H), 1.34-1.49 (m, 1H), 1.53-1.62 (m, 1H), 1.64-1.80 (m, 4H), 2.06-2.18 (m, 1H), 2.52-2.58 (m, 4H), 2.58-2.67 (m, 5H), 2.90 (dd, 2H), 3.07 (dd, 1H), 3.34-3.42 (m, 2H), 4.62-4.75 (m, 1H), 7.38 (d, 2H), 7.57 (d, 2H), 7.62 (d, 2H), 7.71 (d, 2H), 7.87 (m, 4H), 8.11 (d, 1H), 8.17 (s, 1H), 8.42-8.50 (m, 1H), 10.10 (s, 1H).

LC-MS (Method 4): Rₜ=0.63 min; MS (ESIpos): m/z=664.5 [M+H—HCl]⁺.

### Example 92

### 4′-[(2S)-2-({[trans-4-(Aminomethyl)cyclohexyl]carbonyl}amino)-3-{[3-fluoro-4-(2H-tetrazol-5-yl)phenyl]amino}-3-oxopropyl]-N-(3-hydroxycyclopentyl)-2-methylbiphenyl-4-carboxamide hydrochloride

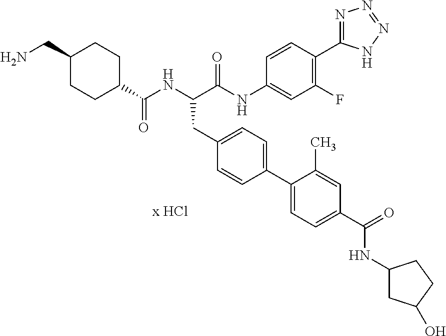

A suspension of 45 mg (0.078 mmol) of tert-butyl [(trans-4-{[(2S)-1-{[3-fluoro-4-(2H-tetrazol-5-yl)phenyl]amino}-3-{4′-[(3-hydroxycyclopentyl)carbamoyl]-2′-methylbiphenyl-4-yl}-1-oxopropan-2-yl]carbamoyl}cyclohexyl)methyl]carbamate in 3.5 ml of dichloromethane was admixed with 0.14 ml (0.78 mmol) of 4M hydrogen chloride in dioxane and stirred at 35° C. overnight. Subsequently, acetonitrile was added. The residue was filtered off and dried under vacuum. This gave 24 mg (56% of theory) of the title compound.

¹H NMR (300 MHz, DMSO-d₆): δ=0.82-0.99 (m, 2H), 1.08-1.21 (m, 1H), 1.22-1.30 (m, 2H), 1.43-1.62 (m, 4H), 1.66-1.80 (m, 6H), 1.81-1.93 (m, 1H), 2.09-2.18 (m, 2H), 2.20 (s, 3H), 2.61 (m, 2H), 2.85-3.00 (m, 1H), 3.08-3.18 (m, 1H), 4.06-4.15 (m, 1H), 4.15-4.27 (m, 1H), 4.67-4.78 (m, 1H), 7.20 (d, 1H), 7.25 (d, 2H), 7.39 (d, 2H), 7.54 (d, 1H), 7.69 (d, 1H), 7.75 (s, 1H), 7.77-7.91 (m, 4H), 8.02 (t, 1H), 8.26-8.35 (m, 2H), 10.81 (s, 1H).

LC-MS (Method 4): Rₜ=0.77 min; MS (ESIpos): m/z=684.6 [M+H—HCl]⁺.

### Example 93

### 4′-{(2S)-2-({[trans-4-(Aminomethyl)cyclohexyl]carbonyl}amino)-3-[(3-chloro-1H-indazol-6-yl)amino]-3-oxopropyl}-2-methyl-N-[(3S)-2-oxopiperidin-3-yl]biphenyl-4-carboxamide hydrochloride

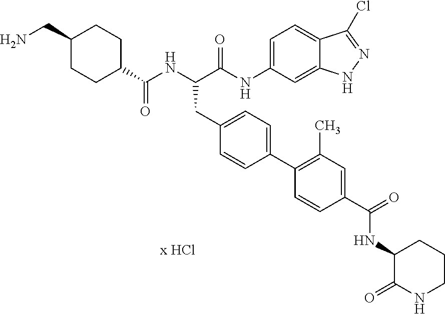

A suspension of 14 mg (0.018 mmol) of tert-butyl [(trans-4-{[(2S)-1-[(3-chloro-1H-indazol-6-yl)amino]-3-(2′-methyl-4′-{[(3S)-2-oxopiperidin-3-yl]carbamoyl}biphenyl-4-yl)-1-oxopropan-2-yl]carbamoyl}cyclohexyl)methyl]carbamate in 1.0 ml of dichloromethane was admixed with 0.045 ml (0.18 mmol) of 4M hydrogen chloride in dioxane and stirred at RT overnight. Subsequently, acetonitrile was added. The residue was filtered off and dried under vacuum. This gave 9 mg (70% of theory) of the title compound.

¹H NMR (300 MHz, DMSO-d₆): δ=0.83-1.01 (m, 2H), 1.07-1.35 (m, 3H), 1.38-1.50 (m, 1H), 1.52-1.62 (m, 1H), 1.66-1.88 (m, 7H), 1.96-2.04 (m, 1H), 2.08-2.17 (m, 1H), 2.21 (s, 3H), 2.24-2.28 (m, 1H), 2.59-2.67 (m, 2H), 2.88-3.00 (m, 1H), 3.08-3.20 (m, 3H), 4.31-4.43 (m, 1H), 4.70-4.81 (m, 1H), 7.25 (m, 4H), 7.37 (d, 2H), 7.57 (d, 1H), 7.63 (s, 1H), 7.66-7.82 (m, 6H), 8.13 (s, 1H), 8.25 (d, 1H), 8.57 (d, 1H), 10.41 (s, 1H), 13.13 (s, 1H).

LC-MS (Method 4): Rₜ=0.84 min; MS (ESIpos): m/z=684.4 [M+H—HCl]⁺.

### Example 94

### 4′-{(2S)-2-({[trans-4-(Aminomethyl)cyclohexyl]carbonyl}amino)-3-[(3-chloro-1H-indazol-6-yl)amino]-3-oxopropyl}-N-[4-(diethylamino)cyclohexyl]-2-methylbiphenyl-4-carboxamide hydrochloride

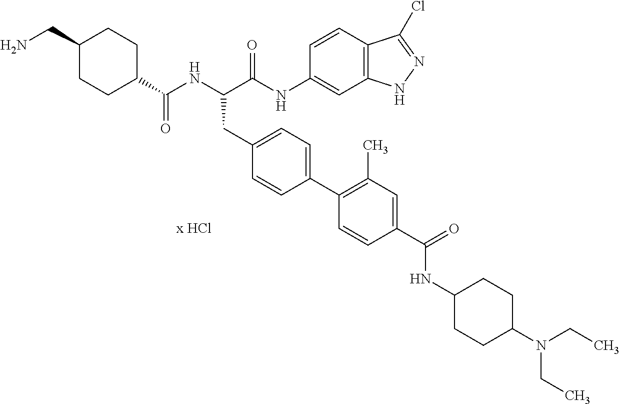

A suspension of 14 mg (0.017 mmol) of tert-butyl [(trans-4-{[(2S)-1-[(3-chloro-1H-indazol-6-yl)amino]-3-(4′-{[4-(diethylamino)cyclohexyl]carbamoyl}-2′-methylbiphenyl-4-yl)-1-oxopropan-2-yl]carbamoyl}cyclohexyl)methyl]carbamate in 1.0 ml of dichloromethane was admixed with 0.04 ml (0.17 mmol) of 4M hydrogen chloride in dioxane and stirred at RT overnight. Subsequently, the reaction mixture was concentrated and the residue was purified by chromatography via HPLC (Method 8). This gave 3 mg (20% of theory) of the title compound.

¹H NMR (300 MHz, DMSO-d₆): δ=0.91-1.00 (m, 6H), 1.11-1.25 (m, 2H), 1.26-1.42 (m, 4H), 1.48-1.62 (m, 2H), 1.66-1.80 (m, 4H), 1.84-1.92 (m, 1H), 2.08-2.15 (m, 1H), 2.19 (s, 3H), 2.55-2.61 (m, 3H), 2.93 (dd, 1H), 3.11 (dd, 1H), 4.66-4.81 (m, 1H), 7.16-7.27 (m, 5H), 7.37 (d, 2H), 7.57 (d, 1H), 7.64-7.71 (m, 1H), 7.71-7.75 (m, 1H), 8.13-8.16 (m, 1H), 8.26-8.38 (m, 2H), 10.43 (s, 1H).

LC-MS (Method 4): Rₜ=0.78 min; MS (ESIpos): m/z=740.4 [M+H—HCl]⁺.

### Example 95

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-cyclopropyl-3-methylbiphenyl-4-carboxamide hydrochloride

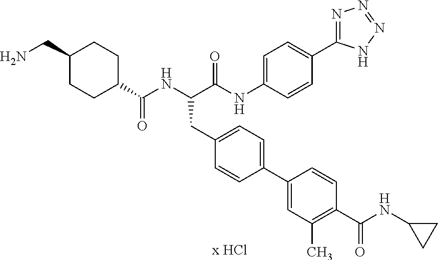

A suspension of 88 mg (0.12 mmol) of tert-butyl [(trans-4-{[(2S)-3-[4′-(cyclopropylcarbamoyl)-3′-methylbiphenyl-4-yl]-1-oxo-1-{[4-(1H-tetrazol-5-yl)phenyl]amino}propan-2-yl]carbamoyl}-cyclohexyl)methyl]carbamate in 3.6 ml of dichloromethane was admixed with 0.37 ml (1.46 mmol) of 4M hydrogen chloride in dioxane and stirred at RT for 48 h. Subsequently, the mixture was admixed with acetonitrile, and the residue was filtered off and dried under reduced pressure. This gave 62 mg (73% of theory) of the title compound.

¹H NMR (400 MHz, DMSO-d₆): δ=0.45-0.53 (m, 2H), 0.61-0.69 (m, 2H), 0.81-0.96 (m, 2H), 1.08-1.30 (m, 2H), 1.38-1.49 (m, 1H), 1.53-1.61 (m, 1H), 1.67-1.78 (m, 3H), 2.07-2.17 (m, 1H), 2.34 (s, 3H), 2.56-2.62 (m, 2H), 2.75-2.84 (m, 1H), 2.91 (dd, 1H), 3.08 (dd, 1H), 4.64-4.74 (m, 1H), 7.30 (d, 1H), 7.37 (d, 2H), 7.41-7.45 (m, 1H), 7.46-7.53 (m, 2H), 7.57 (d, 2H), 7.66-7.77 (m, 3H), 7.80 (d, 2H), 7.97 (d, 2H), 8.19 (d, 1H), 8.24 (d, 1H), 10.49 (s, 1H).

LC-MS (Method 4): Rₜ=0.78 min; MS (ESIpos): m/z=621.5 [M+H—HCl]⁺.

### Example 9

### 4′-[(2S)-2-({[trans-4-(Aminomethyl)cyclohexyl]carbonyl}amino)-3-{[3-fluoro-4-(2H-tetrazol-5-yl)phenyl]amino}-3-oxopropyl]-N-isopropyl-2-methylbiphenyl-4-carboxamide hydrochloride

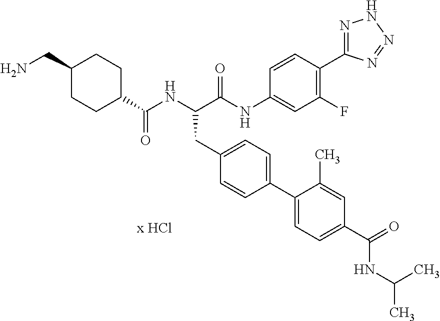

A suspension of 54 mg (0.073 mmol) of tert-butyl {[trans-4-({(2S)-1-{[3-fluoro-4-(2H-tetrazol-5-yl)phenyl]amino}-3-[4′-(isopropylcarbamoyl)-2′-methylbiphenyl-4-yl]-1-oxopropan-2-yl}-carbamoyl)cyclohexyl]methyl}carbamate in 3.6 ml of dichloromethane was admixed with 0.18 ml (0.73 mmol) of 4M hydrogen chloride in dioxane and stirred at 35° C. overnight. Subsequently, the mixture was admixed with acetonitrile, and the residue was filtered off and dried under reduced pressure. This gave 30 mg (59% of theory) of the title compound.

¹H NMR (300 MHz, DMSO-d₆): δ=0.79-1.01 (m, 2H), 1.15 (s, 3H), 1.17 (s, 3H), 1.21-1.32 (m, 2H), 1.39-1.63 (m, 3H), 1.65-1.82 (m, 3H), 2.06-2.16 (m, 1H), 2.20 (s, 3H), 2.58-2.68 (m, 3H), 2.87-3.02 (m, 1H), 3.06-3.17 (m, 1H), 4.01-4.18 (m, 1H), 4.65-4.78 (m, 1H), 7.17-7.30 (m, 3H), 7.38 (d, 2H), 7.48-7.57 (m, 1H), 7.65-7.93 (m, 8H), 8.01 (t, 1H), 8.19 (d, 1H), 8.30 (d, 1H), 10.76 (s, 1H).

LC-MS (Method 4): Rₜ=0.86 min; MS (ESIpos): m/z=642.6 [M+H—HCl]⁺.

### Example 97

### 4′-{(2S)-2-({[trans-4-(Aminomethyl)cyclohexyl]carbonyl}amino)-3-oxo-3-[(2-oxo-2,3-dihydro-1H-benzimidazol-5-yl)amino]propyl}-N-[(3R,5S)-5-(hydroxymethyl)-2-oxopyrrolidin-3-yl]-2-methylbiphenyl-4-carboxamide hydrochloride

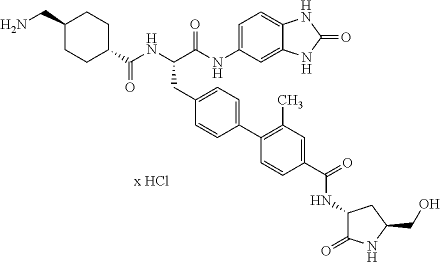 [SMILES: Cc1cc(C(=O)N[C@@H]2C[C@@H](CO)NC2=O)ccc1-c1ccc(C[C@H](NC(=O)[C@H]2CC[C@H](CN)CC2)C(=O)Nc2ccc3[nH]c(=O)[nH]c3c2)cc1]

A solution of 80 mg (0.10 mmol) of tert-butyl {[trans-4-({(2S)-3-(4′-{[(3R,5S)-5-(hydroxymethyl)-2-oxopyrrolidin-3-yl]carbamoyl}-2′-methylbiphenyl-4-yl)-1-oxo-1-[(2-oxo-2,3-dihydro-1H-benzimidazol-5-yl)amino]propan-2-yl}carbamoyl)cyclohexyl]methyl}carbamate in 2 ml of 1,4-dioxane was admixed with 0.05 ml (0.20 mmol) of 4M hydrogen chloride in 1,4-dioxane and stirred at RT for 16 h. Another 0.05 ml (0.20 mmol) of 4M hydrogen chloride in 1,4-dioxane was added, and the mixture was stirred at RT for 18 h. The solvent was removed on a rotary evaporator. After 3 days, 2 ml of 1,4-dioxane and 0.05 ml (0.20 mmol) of 4M hydrogen chloride in 1,4-dioxane were added, and the mixture was stirred at RT for 48 h. Another 0.13 ml (0.50 mmol) of 4M hydrogen chloride in 1,4-dioxane was added, and the mixture was stirred at RT for 16 h. The solid formed was filtered. The residue was washed with acetonitrile and dried under high vacuum. 61 mg (76% of theory, 92% purity) of the title compound were obtained.

¹H NMR (400 MHz, DMSO-d₆) δ ppm 0.80-1.04 (m, 3H), 1.08-1.33 (m, 3H), 1.39-1.60 (m, 3H), 1.66-1.82 (m, 4H), 2.01-2.17 (m, 2H), 2.24 (s, 4H), 2.59-2.68 (m, 2H), 2.86-2.97 (m, 1H), 3.04-3.14 (m, 1H), 3.39 (d, 1H), 4.59-4.77 (m, 2H), 6.84 (d, 1H), 6.99-7.07 (m, 1H), 7.20-7.30 (m, 4H), 7.37 (s, 1H), 7.64-7.82 (m, 6H), 7.91 (s, 1H), 8.16 (d, 1H), 8.62 (d, 1H), 10.02 (s, 1H), 10.51 (s, 1H), 10.58 (s, 1H).

LC-MS (Method 1): Rₜ=0.55 min; MS (ESIneg): m/z=680 [M−H—HCl]⁻.

### Example 98

### 3-{(5-[4-({(2S)-2-({[trans-4-(Aminomethyl)cyclohexyl]carbonyl}amino)-3-[2′-methyl-4′-(piperidin-4-ylcarbamoyl)biphenyl-4-yl]propanoyl}amino)phenyl]-1H-1,2,4-triazol-3-yl}-2,2,3,3-tetrafluoropropanoic acid hydrochloride

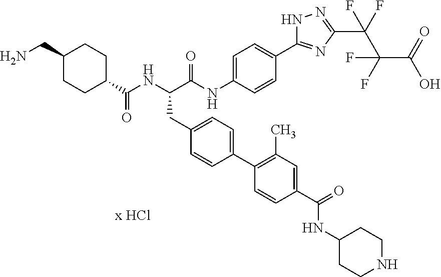 [SMILES: Cc1cc(C(=O)NC2CCNCC2)ccc1-c1ccc(C[C@H](NC(=O)[C@H]2CC[C@H](CN)CC2)C(=O)Nc2ccc(-c3nc(C(F)(F)C(F)(F)C(=O)O)n[nH]3)cc2)cc1]

A solution of 57 mg (0.06 mmol) of 3-[5-(4-{[(2S)-2-{[(trans-4-{[(tert-butoxycarbonyl)amino]-methyl}cyclohexyl)carbonyl]amino}-3-(4′-{[1-(tert-butoxycarbonyl)piperidin-4-yl]carbamoyl}-2′-methylbiphenyl-4-yl)propanoyl]amino}phenyl)-1H-1,2,4-triazol-3-yl]-2,2,3,3-tetrafluoropropanoic acid in 2 ml of 1,4-dioxane was admixed with 0.02 ml (0.85 mmol) of 4M hydrogen chloride in 1,4-dioxane and stirred at RT for 16 h. The solid formed was filtered, washed with acetonitrile and acetonitrile and dried under high vacuum. 51 mg (100% of theory) of the title compound were obtained.

¹H NMR (400 MHz, DMSO-d₆) δ ppm 0.83-1.01 (m, 2H), 1.11-1.34 (m, 2H), 1.39-1.52 (m, 1H), 1.54-1.63 (m, 1H), 1.68-1.83 (m, 6H), 1.92-2.03 (m, 2H), 2.10-2.20 (m, 1H), 2.23 (s, 3H), 2.60-2.70 (m, 2H), 2.90-3.08 (m, 2H), 3.10-3.18 (m, 1H), 3.27-3.37 (m, 2H), 4.00-4.15 (m, 1H), 4.67-4.79 (m, 1H), 7.26 (m, 3H), 7.38 (d, 2H), 7.67-7.84 (m, 7H), 7.96 (d, 2H), 8.27 (d, 1H), 8.47 (d, 1H), 8.58 (br. s, 1H), 8.71 (br. s, 1H), 10.47 (s, 1H), 15.11 (br. s, 1H).

LC-MS (Method 1): Rₜ=0.56 min; MS (ESIneg): m/z=805 [M−H—HCl]⁻.

### Example 99

### 4′-{(2S)-2-({[trans-4-(Aminomethyl)cyclohexyl]carbonyl}amino)-3-[(7-fluoro-2-oxo-2,3-dihydro-1,3-benzoxazol-5-yl)amino]-3-oxopropyl}-2-methyl-N-[(3S)-2-oxopyrrolidin-3-yl]biphenyl-4-carboxamide hydrochloride

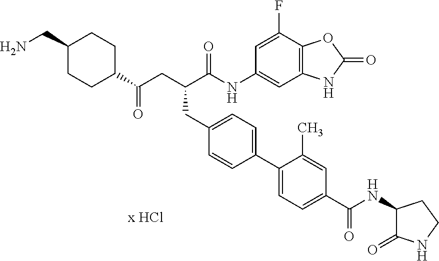

A solution of 32 mg (0.042 mmol) of tert-butyl [(trans-4-{[(2S)-1-[(7-fluoro-2-oxo-2,3-dihydro-1,3-benzoxazol-5-yl)amino]-3-(2′-methyl-4′-{[(3S)-2-oxopyrrolidin-3-yl]carbamoyl}biphenyl-4-yl)-1-oxopropan-2-yl]carbamoyl}cyclohexyl)methyl]carbamate in 1.5 ml of dichloromethane was admixed with 0.05 ml (0.21 mmol) of 4M hydrogen chloride in 1,4-dioxane and stirred at RT overnight and at 40° C. for 2 h. After addition of a further 0.02 ml (0.08 mmol) of 4M hydrogen chloride in 1,4-dioxane and stirring at RT for 2 h, acetonitrile was added, the precipitate was filtered off and washed with a little acetonitrile, and the residue was dried under high vacuum. This gave 22 mg (73% of theory) of the title compound.

¹H NMR (300 MHz, DMSO-d₆): δ=ppm 0.77-1.00 (m, 2H), 1.07-1.34 (m, 2H), 1.38-1.51 (m, 1H), 1.51-1.61 (m, 1H), 1.65-1.82 (m, 3H), 1.94-2.04 (m, 1H), 2.09-2.18 (m, 1H), 2.22 (s, 3H), 2.28-2.40 (m, 1H), 2.57-2.68 (m, 2H), 2.92 (dd, 1H), 3.09 (dd, 1H), 3.19-3.28 (m, 2H), 4.49-4.61 (m, 1H), 4.61-4.72 (m, 1H), 7.26 (m, 6H), 7.37 (d, 2H), 7.67-7.88 (m, 7H), 8.24 (d, 1H), 8.66 (d, 1H), 10.32-10.46 (m, 1H), 11.91-12.00 (m, 1H).

LC-MS (Method 5): Rₜ=0.76 min; MS (ESIpos): m/z=672.4 [M+H]⁺.

### Example 100

### 3-[5-(4-{[(2S)-2-({[trans-4-(Aminomethyl)cyclohexyl]carbonyl}amino)-3-{2′-methyl-4′-[(1-methylpiperidin-4-yl)carbamoyl]biphenyl-4-yl}propanoyl]amino}phenyl)-1H-1,2,4-triazol-3-yl]-2,2,3,3-tetrafluoropropanoic acid hydrochloride

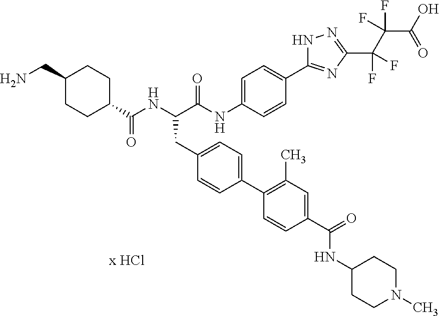

To a solution of 44 mg (47 μmol) of 3-[5-(4-{[(2S)-2-{[(trans-4-{[(tert-butoxycarbonyl)-amino]methyl}cyclohexyl)carbonyl]amino}-3-{2′-methyl-4′-[(1-methylpiperidin-4-yl)carbamoyl]-biphenyl-4-yl}propanoyl]amino}phenyl)-1H-1,2,4-triazol-3-yl]-2,2,3,3-tetrafluoropropanoic acid in 1.5 ml of dioxane was added 0.18 ml (0.71 mmol) of 4M hydrogen chloride in dioxane. The mixture was stirred at RT for 48 h. The precipitated solid was filtered off with suction, washed with acetonitrile and dried under high vacuum. 42 mg (97% of theory) of the title compound were obtained.

¹H NMR (400 MHz, DMSO-d₆): δ=ppm 0.82-1.01 (m, 2H), 1.11-1.33 (m, 2H), 1.43-1.53 (m, 1H), 1.56-1.64 (m, 1H), 1.69-1.82 (m, 4H), 1.86 (m, 2H), 2.00 (m, 2H), 2.10-2.19 (m, 1H), 2.22 (s, 3H), 2.63 (d, 2H), 2.71-2.80 (m, 4H), 2.97 (m, 2H), 3.03-3.17 (m, 4H), 3.26-3.32 (m, 2H), 3.44 (m, 4H), 3.96-4.09 (m, 1H), 4.74 (m, 1H), 7.17-7.29 (m, 3H), 7.39 (d, 2H), 7.67-7.85 (m, 8H), 7.97 (d, 2H), 8.27 (d, 1H), 8.51 (d, 1H), 10.01-10.19 (m, 1H), 10.47 (br. s., 1H), 15.07 (s, 1H).

LC-MS (Method 1): Rₜ=0.58 min; MS (ESIneg): m/z=819.4 [M−H—HCl]⁺.

Alternative Preparation:

To a solution of 7.4 g (8 mmol) of 3-[5-(4-{[(2)-2-{[(trans-4-{[(tert-butoxycarbonyl)amino]-methyl}cyclohexyl)carbonyl]amino}-3-{2′-methyl-4′-[(1-methylpiperidin-4-yl)carbamoyl]-biphenyl-4-yl}propanoyl]amino}phenyl)-1H-1,2,4-triazol-3-yl]-2,2,3,3-tetrafluoropropanoic acid in 80 ml of dioxane were added 30 ml (120 mmol) of 4M hydrogen chloride in dioxane. The mixture was stirred at RT for 3.5 h and treated regularly in an ultrasound bath. 2 ml (8 mmol) of 4M hydrogen chloride in dioxane were added and then stirred at RT for 8 h and treated regularly in an ultrasound bath. The solid was filtered off with suction, washed repeatedly with diethyl ether and dried under high vacuum. 7.1 g (99% of theory) of the title compound were obtained.

¹H NMR (400 MHz, DMSO-d₆): δ=ppm 0.76-1.02 (m, 2H), 1.12-1.35 (m, 2H), 1.39-1.64 (m, 3H), 1.67-1.93 (m, 4H), 2.00 (m, 2H), 2.10-2.20 (m, 1H), 2.23 (s, 3H), 2.57-2.68 (m, 2H), 2.74 (m, 3H), 2.89-3.01 (m, 1H), 3.03-3.18 (m, 3H), 3.41-3.47 (m, 2H), 3.95-4.09 (m, 1H), 4.62-4.83 (m, 1H), 7.07-7.31 (m, 3H), 7.39 (d, 2H), 7.63-7.87 (m, 6H), 7.98 (d, 2H), 8.26 (d, 1H), 8.50 (d, 1H), 10.12 (br. s, 1H), 10.47 (s, 1H), 15.12 (br. s, 1H).

LC-MS (Method 12): Rₜ=1.59 min; MS (ESIpos): m/z=821.4 [M+H—HCl]⁺.

### Example 101

### 3-[5-(4-{[(2S)-2-({[trans-4-(Aminomethyl)cyclohexyl]carbonyl}amino)-3-(4′-{[4-(dimethyl-amino)cyclohexyl]carbamoyl}-2′-methylbiphenyl-4-yl)propanoyl]amino}phenyl)-1H-1,2,4-triazol-3-yl]-2,2,3,3-tetrafluoropropanoic acid hydrochloride

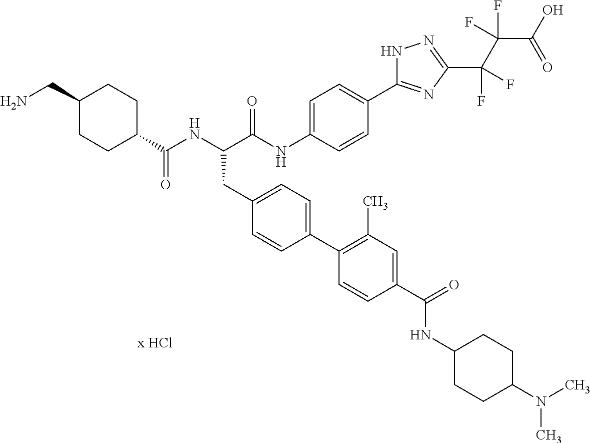

To a solution of 13 mg (13 μmol) of 3-[5-(4-{[(2S)-2-{[(trans-4-{[(tert-butoxycarbonyl)amino]-methyl}cyclohexyl)carbonyl]amino}-3-(4′-{[4-(dimethylamino)cyclohexyl]carbamoyl}-2′-methylbiphenyl-4-yl)propanoyl]amino}phenyl)-1H-1,2,4-triazol-3-yl]-2,2,3,3-tetrafluoropropanoic acid in 0.6 ml of dioxane was added 0.05 ml (0.2 mmol) of 4M hydrogen chloride in dioxane. The mixture was stirred at RT for 16 h. The precipitated solid was filtered off with suction, washed with acetonitrile and dried under high vacuum. 8 mg (63% of theory) of the title compound were obtained.

¹H NMR (400 MHz, DMSO-d₆): δ=ppm 0.83-0.99 (m, 2H), 1.13-1.32 (m, 2H), 1.36-1.51 (m, 3H), 1.52-1.64 (m, 3H), 1.69-1.87 (m, 3H), 1.92-2.01 (m, 2H), 2.01-2.09 (m, 2H), 2.11-2.19 (m, 1H), 2.22 (s, 3H), 2.24-2.29 (m, 1H), 2.59-2.65 (m, 2H), 2.73 (m, 6H), 2.88-2.99 (m, 2H), 3.09-3.17 (m, 3H), 3.76-3.87 (m, 2H), 4.67-4.79 (m, 1H), 7.26 (m, 3H), 7.39 (d, 2H), 7.76 (m, 6H), 7.97 (d, 2H), 8.24-8.34 (m, 2H), 9.89-10.01 (m, 1H), 10.43-10.51 (m, 1H), 15.02-15.18 (m, 1H).

LC-MS (Method 1): Rₜ=0.58 min; MS (ESIpos): m/z=849.5 [M+H—HCl]⁺.

### Example 102

### 4′-{(2S)-2-({[trans-4-(Aminomethyl)cyclohexyl]carbonyl}amino)-3-[(7-chloro-2-oxo-2,3-dihydro-1,3-benzoxazol-5-yl)amino]-3-oxopropyl}-N-cyclobutyl-2-methylbiphenyl-4-carboxamide hydrochloride

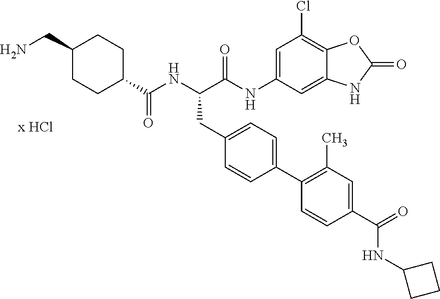 [SMILES: Cc1cc(C(=O)NC2CCC2)ccc1-c1ccc(C[C@H](NC(=O)[C@H]2CC[C@H](CN)CC2)C(=O)Nc2cc(Cl)c3oc(=O)[nH]c3c2)cc1]

A suspension of tert-butyl {[trans-4-({(2S)-1-[(7-chloro-2-oxo-2,3-dihydro-1,3-benzoxazol-5-yl)amino]-3-[4′-(cyclobutylcarbamoyl)-2′-methylbiphenyl-4-yl]-1-oxopropan-2-yl}carbamoyl)-cyclohexyl]methyl}carbamate (27 mg, 0.036 mmol) in dioxane (1.5 ml) was admixed with 4M hydrogen chloride in 1,4-dioxane (0.13 ml, 0.53 mmol) and stirred at RT for 8 days. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 16 mg (65% of theory) of the title compound.

¹H NMR (400 MHz, DMSO-d₆): δ=ppm 0.85-1.00 (m, 2H), 1.12-1.34 (m, 3H), 1.47 (br. s, 1H), 1.59 (d, 1H), 1.64-1.82 (m, 5H), 2.01-2.16 (m, 3H), 2.22 (s, 4H), 2.60-2.69 (m, 2H), 2.94 (dd, 1H), 3.09 (dd, 1H), 4.35-4.51 (m, 1H), 4.61-4.72 (m, 1H), 7.21 (d, 1H), 7.25 (d, 2H), 7.34-7.39 (m, 3H), 7.43 (d, 1H), 7.66-7.78 (m, 5H), 8.22 (d, 1H), 8.58 (d, 1H), 10.31 (s, 1H), 11.92 (s, 1H).

LC-MS (Method 1): Rₜ=0.80 min; MS (ESIneg): m/z=656 [M−H—HCl]⁻.

### Example 103

### 3-[5-(4-{[2-({[trans-4-(Aminomethyl)cyclohexyl]carbonyl}amino)-3-{4′-[(trans-4-hydroxycyclohexyl)carbamoyl]-2′-methylbiphenyl-4-yl}propanoyl]amino}phenyl)-4H-1,2,4-triazol-3-yl]-2,2,3,3-tetrafluoropropanoic acid hydrochloride (enantiomer mixture)

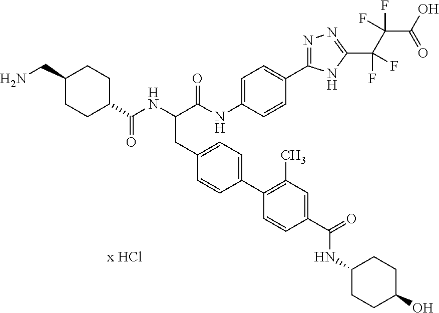 [SMILES: Cc1cc(C(=O)N[C@H]2CC[C@H](O)CC2)ccc1-c1ccc(CC(NC(=O)[C@H]2CC[C@H](CN)CC2)C(=O)Nc2ccc(-c3nnc(C(F)(F)C(F)(F)C(=O)O)[nH]3)cc2)cc1]

To a solution of 7 g (6.8 mol) of 3-[5-(4-{[(2S)-2-{[(trans-4-{[(tert-butoxycarbonyl)amino]-methyl}cyclohexyl)carbonyl]amino}-3-{4′-[(trans-4-{[tert-butyl(dimethyl)silyl]oxy}cyclohexyl)-carbamoyl]-2′-methylbiphenyl-4-yl}propanoyl]amino}phenyl)-4H-1,2,4-triazol-3-yl]-2,2,3,3-tetrafluoropropanoic acid in 215 ml of dioxane was added 17 ml (68 mmol) of 4M hydrogen chloride in dioxane. The mixture was stirred at RT for 7 h, partly in an ultrasound bath. 8.5 ml (34 mmol) of 4M hydrogen chloride in dioxane were added, and the mixture was stirred at RT for 18 h. 200 ml of acetonitrile were added and the precipitated solid was filtered off. The solid was washed with acetonitrile and diethyl ether and dried under high vacuum. 6 g (98% of theory) of the title compound were obtained.

¹H NMR (400 MHz, DMSO-d₆): δ=ppm 0.82-0.99 (m, 2H), 1.13-1.30 (m, 4H), 1.30-1.52 (m, 3H), 1.53-1.61 (m, 1H), 1.69-1.90 (m, 7H), 2.09-2.18 (m, 1H), 2.22 (s, 3H), 2.58-2.68 (m, 2H), 2.91-3.01 (m, 1H), 3.10-3.17 (m, 1H), 3.66-3.79 (m, 1H), 4.64-4.82 (m, 1H), 7.20 (d, 1H), 7.25 (d, 2H), 7.39 (d, 2H), 7.66-7.84 (m, 7H), 7.97 (d, 2H), 8.13 (d, 1H), 8.24 (d, 1H), 10.50 (s, 1H), 15.13 (br. s, 1H).

LC-MS (Method 1): Rₜ=0.67 min; MS (ESIpos): m/z=822.2 [M+H—HCl]⁺.

### Example 104

### 3-[5-(4-{[(2S)-2-({[trans-4-(Aminomethyl)cyclohexyl]carbonyl}amino)-3-{4′-[(trans-4-hydroxycyclohexyl)carbamoyl]-2′-methylbiphenyl-4-yl}propanoyl]amino}phenyl)-4H-1,2,4-triazol-3-yl]-2,2,3,3-tetrafluoropropanoic acid hydrochloride (enantiomer 1)

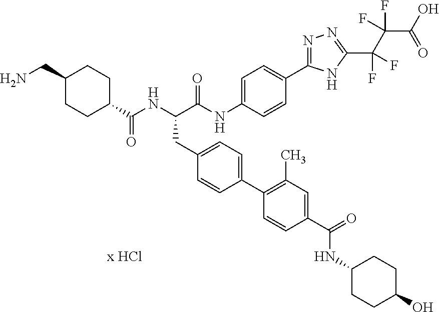 [SMILES: Cc1cc(C(=O)N[C@H]2CC[C@H](O)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

3-[5-(4-{[(2S)-2-({[trans-4-(Aminomethyl)cyclohexyl]carbonyl}amino)-3-{4′-[(trans-4-hydroxycyclohexyl)carbamoyl]-2′-methylbiphenyl-4-yl}propanoyl]amino}phenyl)-4H-1,2,4-triazol-3-yl]-2,2,3,3-tetrafluoropropanoic acid (enantiomer 1) (610 mg, 0.74 mmol) was stirred with 2 ml of 4M hydrogen chloride in dioxane at RT for 15 min. Subsequently, the mixture was concentrated fully and the residue was dried under high vacuum. 636 mg (99% of theory) of the title compound were obtained.

Chiral analytical HPLC: Rₜ=8.23 min; >97% ee.

Specific rotation: [α]=46.9° (c=0.420 g/100 ml, methanol, 20° C., 589 nm).

Analysis: column: Daicel Chiralpak ID 5μ 20 mm×250 mm; eluent: 65% isohexane, 35% ethanol+5 g/L (−)-camphorsulphonic acid; flow rate: 1 ml/min; UV detection: 260 nm.

### Example 105

### 3-[5-(4-{[(2R)-2-({[trans-4-(Aminomethyl)cyclohexyl]carbonyl}amino)-3-{4′-[(trans-4-hydroxycyclohexyl)carbamoyl]-2′-methylbiphenyl-4-yl}propanoyl]amino}phenyl)-4H-1,2,4-triazol-3-yl]-2,2,3,3-tetrafluoropropanoic acid (enantiomer 2)

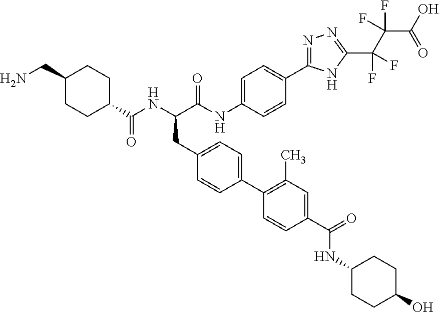 [SMILES: Cc1cc(C(=O)N[C@H]2CC[C@H](O)CC2)ccc1-c1ccc(C[C@@H](NC(=O)[C@H]2CC[C@H](CN)CC2)C(=O)Nc2ccc(-c3nnc(C(F)(F)C(F)(F)C(=O)O)[nH]3)cc2)cc1]

In analogy to the synthesis of Example 169, using methyl 3-[5-(4-{[(2R)-2-{[(trans-4-{[(tert-butoxycarbonyl)amino]methyl}cyclohexyl)carbonyl]-amino}-3-{4′-[(trans-4-{[tert-butyl(dimethyl)silyl]oxy}cyclohexyl)carbamoyl]-2′-methylbiphenyl-4-yl}propanoyl]-amino}phenyl)-4H-1,2,4-triazol-3-yl]-2,2,3,3-tetrafluoropropanoate (enantiomer 2) (32 mg, 0.03 mmol), 15 mg (54% of theory) of the title compound were obtained.

Chiral analytical HPLC: Rₜ=7.21 min; >97% ee.

Specific rotation: [α]=−54.9° (c=0.241 g/100 ml, DMSO, 20° C., 589 nm).

Analysis: column: Daicel Chiralpak ID 5 μm 20 mm×250 mm; eluent: 65% isohexane, 35% ethanol+5 g/L (−)-camphorsulphonic acid; flow rate: 1 ml/min; UV detection: 260 nm.

### Example 106

### 3-[5-(4-{[(2S)-2-({[trans-4-(Aminomethyl)cyclohexyl]carbonyl}amino)-3-(4′-{[(3R,5S)-5-(hydroxymethyl)-2-oxopyrrolidin-3-yl]carbamoyl}-2′-methylbiphenyl-4-yl)propanoyl]amino}-phenyl)-1H-1,2,4-triazol-3-yl]-2,2,3,3-tetrafluoropropanoic acid hydrochloride

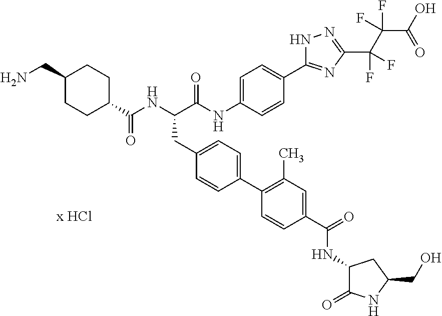 [SMILES: Cc1cc(C(=O)N[C@@H]2C[C@@H](CO)NC2=O)ccc1-c1ccc(C[C@H](NC(=O)[C@H]2CC[C@H](CN)CC2)C(=O)Nc2ccc(-c3nc(C(F)(F)C(F)(F)C(=O)O)n[nH]3)cc2)cc1]

To a solution of 20 mg (21 μmol) of 3-[5-(4-{[(2S)-2-{[(trans-4-{[(tert-butoxycarbonyl)amino]methyl}cyclohexyl)carbonyl]amino}-3-(4′-{[(3R,5S)-5-(hydroxymethyl)-2-oxopyrrolidin-3-yl]carbamoyl}-2′-methylbiphenyl-4-yl)propanoyl]amino}phenyl)-1H-1,2,4-triazol-3-yl]-2,2,3,3-tetrafluoropropanoic acid in 0.72 ml of dioxane were added 27 μl (107 μmol) of 4M hydrogen chloride in dioxane. The mixture was stirred at RT for 18 h. The solvent was removed and the solid obtained was dried under high vacuum. 19 mg (97% of theory) of the title compound were obtained.

¹H NMR (400 MHz, DMSO-d₆): δ=ppm 0.78-1.03 (m, 2H), 1.10-1.36 (m, 3H), 1.40-1.65 (m, 2H), 1.68-1.84 (m, 3H), 2.01-2.28 (m, 6H), 2.59-2.69 (m, 2H), 2.88-2.98 (m, 1H), 3.06-3.18 (m, 1H), 3.39 (d, 2H), 4.57-4.70 (m, 1H), 4.71-4.85 (m, 1H), 7.28 (m, 3H), 7.39 (d, 2H), 7.67-7.83 (m, 6H), 7.85-7.93 (m, 2H), 7.98 (d, 2H), 8.27 (d, 1H), 8.63 (d, 1H), 8.86 (d, 1H), 10.49 (s, 1H), 15.15 (br. s, 1H).

LC-MS (Method 1): Rₜ=0.61 min; MS (ESIpos): m/z=837.6 [M+H—HCl]⁺.

### Example 107

### 3-[5-(4-{[(2S)-2-({[trans-4-(Aminomethyl)cyclohexyl]carbonyl}amino)-3-{4′-[(3,3-dimethylpiperidin-4-yl)carbamoyl]-2′-methylbiphenyl-4-yl}propanoyl]amino}phenyl)-4H-1,2,4-triazol-3-yl]-2,2,3,3-tetrafluoropropanoic acid hydrochloride

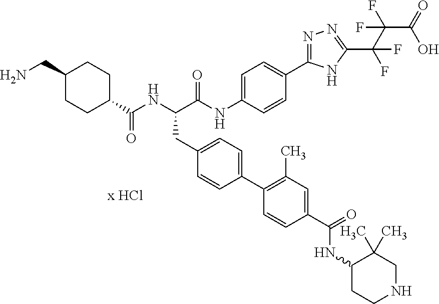 [SMILES: Cc1cc(C(=O)NC2CCNCC2(C)C)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

To a solution of 52 mg (50 μmol) of 3-[5-(4-{[(2S)-2-{[(trans-4-{[(tert-butoxycarbonyl)amino]methyl}cyclohexyl)carbonyl]amino}-3-(4′-{[1-(tert-butoxycarbonyl)-3,3-dimethylpiperidin-4-yl]carbamoyl}-2′-methylbiphenyl-4-yl)propanoyl]amino}phenyl)-4H-1,2,4-triazol-3-yl]-2,2,3,3-tetrafluoropropanoic acid in 1 ml of dioxane were added 189 μl (76 μmol) of 4M hydrogen chloride in dioxane. The mixture was stirred at RT for 18 h. Acetonitrile was added and the solid obtained was filtered, washed with acetonitrile and dried under high vacuum. 43 mg (94% of theory) of the title compound were obtained.

¹H NMR (400 MHz, DMSO-d₆): δ=ppm 0.84-0.99 (m, 5H), 1.06 (s, 3H), 1.15-1.33 (m, 2H), 1.40-1.63 (m, 2H), 1.74 (m, 3H), 1.89-2.02 (m, 1H), 2.11-2.18 (m, 1H), 2.24 (s, 3H), 2.56-2.71 (m, 3H), 2.83-3.02 (m, 3H), 3.04-3.20 (m, 1H), 3.25 (m, 1H), 4.08-4.20 (m, 1H), 4.64-4.82 (m, 1H), 7.15-7.32 (m, 3H), 7.41 (d, 2H), 7.63-7.93 (m, 6H), 7.99 (d, 2H), 8.17 (d, 1H), 8.30 (d, 1H), 8.57 (br. s, 1H), 9.12 (br. s, 1H), 10.55 (br. s., 1H), 15.22 (br. s, 1H).

LC-MS (Method 1): Rₜ=0.59 min; MS (ESIpos): m/z=835.5 [M+H—HCl]⁺.

### Example 108

### 3-[5-(4-{[(2S)-2-({[trans-4-(Aminomethyl)cyclohexyl]carbonyl}amino)-3-(2′-methyl-4′-{[(1-methylpiperidin-4-yl)methyl]carbamoyl}biphenyl-4-yl)propanoyl]amino}phenyl)-4H-1,2,4-triazol-3-yl]-2,2,3,3-tetrafluoropropanoic acid hydrochloride

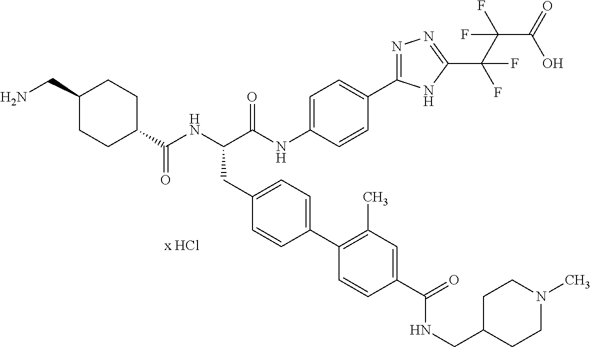 [SMILES: Cc1cc(C(=O)NCC2CCN(C)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

To a solution of 63 mg (67 μmol) of 3-[5-(4-{[(2S)-2-{[(trans-4-{[(tert-Butoxycarbonyl)amino]methyl}cyclohexyl)carbonyl]amino}-3-(2′-methyl-4′-{[(1-methylpiperidin-4-yl)methyl]carbamoyl}biphenyl-4-yl)propanoyl]amino}phenyl)-4H-1,2,4-triazol-3-yl]-2,2,3,3-tetrafluoropropanoic acid in 1 ml of dioxane were added 252 μl (1 mmol) of 4M hydrogen chloride in dioxane. The mixture was stirred at RT for 18 h. Acetonitrile was added and the solid obtained was filtered, washed with acetonitrile and dried under high vacuum. 31 mg (51% of theory) of the title compound were obtained.

¹H NMR (400 MHz, DMSO-d₆): δ=ppm 0.81-1.02 (m, 2H), 1.07-1.34 (m, 2H), 1.45 (m, 4H), 1.64-1.90 (m, 6H), 2.12-2.19 (m, 1H), 2.23 (s, 3H), 2.59-2.67 (m, 2H), 2.71 (d, 2H), 2.81-3.02 (m, 3H), 3.07-3.23 (m, 4H), 3.39 (d, 3H), 4.67-4.84 (m, 1H), 7.17-7.30 (m, 3H), 7.40 (d, 2H), 7.65-7.90 (m, 6H), 7.98 (d, 2H), 8.28 (d, 1H), 8.50-8.66 (m, 1H), 9.92 (br. s, 1H), 10.51 (br. s., 1H), 15.18 (br. s, 1H).

LC-MS (Method 1): Rₜ=0.57 min; MS (ESIpos): m/z=835.5 [M+H—HCl]⁺.

### Example 109

### 4′-[(2S)-2-({[trans-4-(Aminomethyl)cyclohexyl]carbonyl}amino)-3-oxo-3-({4-[3-(1,1,2,2-tetrafluoro-3-hydroxypropyl)-1H-1,2,4-triazol-5-yl]phenyl}amino)propyl]-2-methyl-N-(1-methylpiperidin-4-yl)biphenyl-4-carboxamide hydrochloride

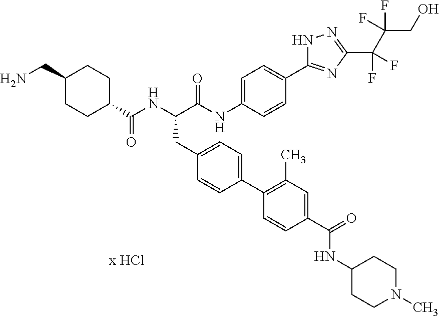 [SMILES: Cc1cc(C(=O)NC2CCN(C)CC2)ccc1-c1ccc(C[C@H](NC(=O)[C@H]2CC[C@H](CN)CC2)C(=O)Nc2ccc(-c3nc(C(F)(F)C(F)(F)CO)n[nH]3)cc2)cc1]

To a solution of 55.8 mg (62 μmol) of tert-butyl [(trans-4-{[(2S)-3-{2′-methyl-4′-[(1-methylpiperidin-4-yl)carbamoyl]biphenyl-4-yl}-1-oxo-1-({4-[3-(1,1,2,2-tetrafluoro-3-hydroxypropyl)-1H-1,2,4-triazol-5-yl]phenyl}amino)propan-2-yl]carbamoyl}cyclohexyl)-methyl]carbamate in 2 ml of dioxane were added 154 μl (0.62 mmol) of 4M hydrogen chloride in dioxane. The mixture was stirred at RT for 18 h. Acetonitrile was added and the solid obtained was filtered, washed with acetonitrile and dried under high vacuum. 46 mg (78% of theory) of the title compound were obtained.

¹H NMR (400 MHz, DMSO-d₆): δ=ppm 0.78-1.02 (m, 2H), 1.11-1.37 (m, 2H), 1.42-1.63 (m, 2H), 1.74 (m, 3H), 1.85-2.05 (m, 4H), 2.10-2.20 (m, 1H), 2.24 (s, 3H), 2.63 (m, 2H), 2.73 (m, 3H), 2.89-3.19 (m, 4H), 3.43 (m, 2H), 4.03 (m, 3H), 4.66-4.80 (m, 1H), 7.20-7.30 (m, 3H), 7.40 (d, 2H), 7.71-7.90 (m, 7H), 8.00 (d, 2H), 8.27 (d, 1H), 8.52 (d, 1H), 10.29 (br. s, 1H), 10.51 (br. s., 1H), 15.11 (br. s, 1H).

LC-MS (Method 1): Rₜ=0.59 min; MS (ESIneg): m/z=805.2 [M−H—HCl]⁻.

### Example 110

### 4′-[(2S)-2-({[trans-4-(Aminomethyl)cyclohexyl]carbonyl}amino)-3-oxo-3-({4-[3-(1,1,2,2-tetrafluoro-3-hydroxypropyl)-1H-1,2,4-triazol-5-yl]phenyl}amino)propyl]-N-(trans-4-hydroxycyclohexyl)-2-methylbiphenyl-4-carboxamide hydrochloride

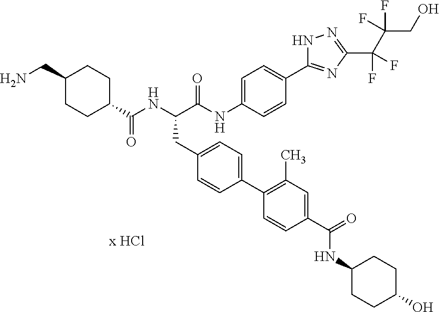 [SMILES: Cc1cc(C(=O)N[C@H]2CC[C@H](O)CC2)ccc1-c1ccc(C[C@H](NC(=O)[C@H]2CC[C@H](CN)CC2)C(=O)Nc2ccc(-c3nc(C(F)(F)C(F)(F)CO)n[nH]3)cc2)cc1]

To a solution of 57.9 mg (64 μmol) of tert-butyl [(trans-4-{[(2S)-3-{4′-[(trans-4-hydroxycyclohexyl)carbamoyl]-2′-methylbiphenyl-4-yl}-1-oxo-1-({4-[3-(1,1,2,2-tetrafluoro-3-hydroxypropyl)-1H-1,2,4-triazol-5-yl]phenyl}amino)propan-2-yl]carbamoyl}cyclohexyl)-methyl]carbamate in 2 ml of dioxane were added 159 μl (0.64 mmol) of 4M hydrogen chloride in dioxane. The mixture was stirred at RT for 18 h. Acetonitrile was added and the solid obtained was filtered, washed with acetonitrile and dried under high vacuum. The residue was admixed once again with 2 ml of dioxane and 159 μl (0.64 mmol) of 4M hydrogen chloride in dioxane. The mixture was stirred at RT for 18 h. Acetonitrile was added and the solid obtained was filtered, washed with acetonitrile and dried under high vacuum. 31 mg (55% of theory) of the title compound were obtained.

¹H NMR (400 MHz, DMSO-d₆): δ=ppm 0.79-1.02 (m, 2H), 1.10-1.62 (m, 9H), 1.66-1.90 (m, 7H), 2.08-2.19 (m, 1H), 2.22 (s, 3H), 2.63 (m, 2H), 2.88-3.00 (m, 1H), 3.08-3.19 (m, 1H), 3.32-3.45 (m, 1H), 3.67-3.79 (m, 1H), 4.03 (t, 2H), 4.66-4.82 (m, 1H), 7.14-7.30 (m, 3H), 7.39 (d, 2H), 7.65-7.87 (m, 7H), 7.99 (d, 2H), 8.14 (d, 1H), 8.25 (d, 1H), 10.49 (br. s., 1H), 15.07 (br. s, 1H).

LC-MS (Method 1): Rₜ=0.70 min; MS (ESIpos): m/z=808.4 [M+H—HCl]⁺.

### Example 111

### 3-[3-(4-{[(2S)-2-({[trans-4-(Aminomethyl)cyclohexyl]carbonyl}amino)-3-(3′-fluoro-4′-{[(3R)-2-oxopiperidin-3-yl]carbamoyl}biphenyl-4-yl)propanoyl]amino}phenyl)-1H-1,2,4-triazol-5-yl]-2,2,3,3-tetrafluoropropanoic acid hydrochloride

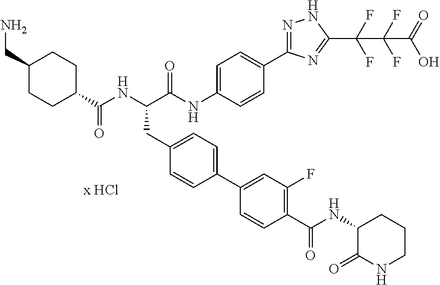 [SMILES: NC[C@H]1CC[C@H](C(=O)N[C@@H](Cc2ccc(-c3ccc(C(=O)N[C@@H]4CCCNC4=O)c(F)c3)cc2)C(=O)Nc2ccc(-c3n[nH]c(C(F)(F)C(F)(F)C(=O)O)n3)cc2)CC1]

To a solution of 52 mg (56 μmol) of 3-[3-(4-{[(2S)-2-{[(trans-4-{[(tert-butoxycarbonyl)amino]methyl}cyclohexyl)carbonyl]amino}-3-(3′-fluoro-4′-{[(3R)-2-oxopiperidin-3-yl]carbamoyl}biphenyl-4-yl)propanoyl]amino}phenyl)-1H-1,2,4-triazol-5-yl]-2,2,3,3-tetrafluoropropanoic acid in 2 ml of dioxane were added 70 μl (0.28 mmol) of 4M hydrogen chloride in dioxane. The mixture was stirred at RT for 18 h. The solvent was removed and the residue was dried under high vacuum. 50 mg (99% of theory) of the title compound were obtained.

¹H NMR (400 MHz, DMSO-d₆): δ=ppm 0.76-1.00 (m, 2H), 1.10-1.33 (m, 3H), 1.39-1.51 (m, 1H), 1.55-1.64 (m, 1H), 1.67-1.90 (m, 6H), 2.05-2.22 (m, 2H), 2.59-2.77 (m, 3H), 2.87-2.98 (m, 1H), 3.08-3.22 (m, 2H), 4.25-4.40 (m, 1H), 4.65-4.77 (m, 1H), 7.42 (d, 2H), 7.56-7.86 (m, 10H), 7.97 (d, 2H), 8.21 (d, 1H), 8.35 (d, 1H), 10.49 (s, 1H), 15.01 (br. s, 1H).

LC-MS (Method 1): Rₜ=0.67 min; MS (ESIpos): m/z=825.4 [M+H—HCl]⁺.

### Example 112

### 3-[3-(4-{[(2S)-2-({[trans-4-(Aminomethyl)cyclohexyl]carbonyl}amino)-3-{3′-fluoro-4′-[(trans-4-hydroxycyclohexyl)carbamoyl]biphenyl-4-yl}propanoyl]amino}phenyl)-1H-1,2,4-triazol-5-yl]-2,2,3,3-tetrafluoropropanoic acid hydrochloride

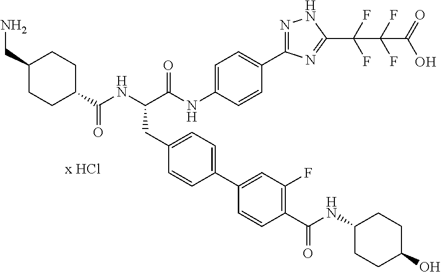 [SMILES: NC[C@H]1CC[C@H](C(=O)N[C@@H](Cc2ccc(-c3ccc(C(=O)N[C@H]4CC[C@H](O)CC4)c(F)c3)cc2)C(=O)Nc2ccc(-c3n[nH]c(C(F)(F)C(F)(F)C(=O)O)n3)cc2)CC1]

To a solution of 17 mg (18 μmol) of 3-[3-(4-{[(2S)-2-{[(trans-4-{[(tert-butoxycarbonyl)amino]methyl}cyclohexyl)carbonyl]amino}-3-{3′-fluoro-4′-[(trans-4-hydroxycyclohexyl)carbamoyl]biphenyl-4-yl}propanoyl]amino}phenyl)-1H-1,2,4-triazol-5-yl]-2,2,3,3-tetrafluoropropanoic acid in 1 ml of dioxane were added 23 μl (0.09 mmol) of 4M hydrogen chloride in dioxane. The mixture was stirred at RT for 18 h. Another 23 μl (0.09 mmol) of 4M hydrogen chloride in dioxane were added and the mixture was stirred at RT for 3 h. The solvent was removed and the residue was dried under high vacuum. 16 mg (99% of theory) of the title compound were obtained.

¹H NMR (400 MHz, DMSO-d₆): δ=ppm 0.74-1.00 (m, 2H), 1.11-1.51 (m, 8H), 1.55-1.66 (m, 1H), 1.68-1.90 (m, 6H), 2.07-2.21 (m, 1H), 2.58-2.69 (m, 2H), 2.87-2.98 (m, 1H), 3.07-3.17 (m, 1H), 3.63-3.76 (m, 2H), 4.67-4.78 (m, 1H), 7.42 (d, 2H), 7.69 (m, 7H), 7.79 (d, 2H), 7.96 (d, 2H), 8.09 (d, 1H), 8.21 (d, 1H), 10.48 (s, 1H), 15.03 (br. s, 1H).

LC-MS (Method 1): Rₜ=0.67 min; MS (ESIpos): m/z=826.5 [M+H—HCl]⁺.

### Example 113

### 3-[5-(4-{[(2S)-2-({[trans-4-(Aminomethyl)cyclohexyl]carbonyl}amino)-3-{2′-methyl-4′-[methyl(1-methylpiperidin-4-yl)carbamoyl]biphenyl-4-yl}propanoyl]amino}phenyl)-4H-1,2,4-triazol-3-yl]-2,2,3,3-tetrafluoropropanoic acid hydrochloride

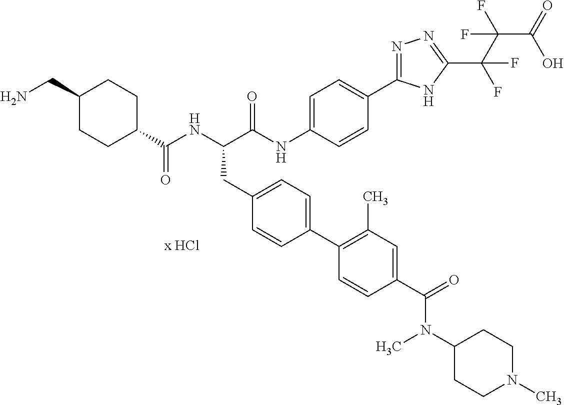 [SMILES: Cc1cc(C(=O)N(C)C2CCN(C)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

To a solution of 23 mg (24 μmol) of 3-[5-(4-{[(2S)-2-{[(trans-4-{[(tert-butoxycarbonyl)amino]methyl}cyclohexyl)carbonyl]amino}-3-{2′-methyl-4′-[methyl(1-methylpiperidin-4-yl)carbamoyl]biphenyl-4-yl}propanoyl]amino}phenyl)-4H-1,2,4-triazol-3-yl]-2,2,3,3-tetrafluoropropanoic acid in 2 ml of dioxane were added 92 μl (0.37 mmol) of 4M hydrogen chloride in dioxane. The mixture was stirred at RT for 18 h. Acetonitrile was added and the solid obtained was filtered, washed with acetonitrile and dried under high vacuum. 23 mg (98% of theory) of the title compound were obtained.

¹H NMR (400 MHz, DMSO-d₆): δ=ppm 0.76-1.03 (m, 2H), 1.10-1.32 (m, 3H), 1.39-1.52 (m, 1H), 1.55-1.62 (m, 1H), 1.68-1.93 (m, 5H), 2.06-2.19 (m, 2H), 2.21 (s, 3H), 2.59-2.76 (m, 5H), 2.81 (s, 3H), 2.90-3.00 (m, 2H), 3.07-3.17 (m, 2H), 3.41-3.54 (m, 3H), 4.66-4.81 (m, 1H), 7.13-7.34 (m, 5H), 7.39 (d, 2H), 7.67-7.82 (m, 5H), 7.97 (d, 2H), 8.25 (d, 1H), 10.11 (br. s, 1H), 10.47 (s, 1H), 15.12 (br. s, 1H).

LC-MS (Method 1): Rₜ=0.54 min; MS (ESIpos): m/z=835.4 [M+H—HCl]⁺.

### Example 114

### 3-{5-[4-({(2S)-2-({[trans-4-(Aminomethyl)cyclohexyl]carbonyl}amino)-3-[4′-{(2-[4-(dimethylamino)piperidin-1-yl]-2-oxoethyl}carbamoyl)-2′-methylbiphenyl-4-yl]propanoyl}-amino)phenyl]-4H-1,2,4-triazol-3-yl}-2,2,3,3-tetrafluoropropanoic acid hydrochloride

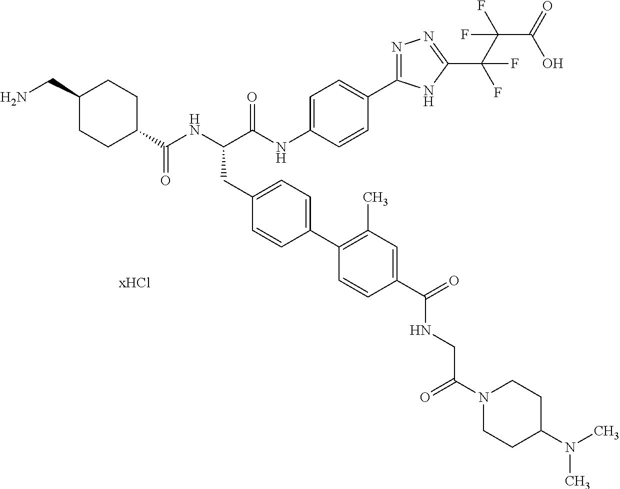 [SMILES: Cc1cc(C(=O)NCC(=O)N2CCC(N(C)C)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

To a solution of 67 mg (67 μmol) of 3-{5-[4-({(2S)-2-{[(trans-4-{[(tert-butoxycarbonyl)amino]methyl}cyclohexyl)carbonyl]amino}-3-[4′-({2-[4-(dimethylamino)-piperidin-1-yl]-2-oxoethyl}carbamoyl)-2′-methylbiphenyl-4-yl]propanoyl}amino)phenyl]-4H-1,2,4-triazol-3-yl}-2,2,3,3-tetrafluoropropanoic acid in 1 ml of dioxane were added 252 μl (1 mmol) of 4M hydrogen chloride in dioxane. The mixture was stirred at RT for 18 h. Acetonitrile was added and the solid obtained was filtered, washed with acetonitrile and dried under high vacuum. 64 mg (91% of theory) of the title compound were obtained.

¹H NMR (400 MHz, DMSO-d₆): δ=ppm 0.86-1.01 (m, 2H), 1.12-1.38 (m, 2H), 1.41-1.53 (m, 1H), 1.60 (s, 3H), 1.70-1.83 (m, 3H), 2.01-2.20 (m, 1H), 2.24 (s, 3H), 2.64 (m, 2H), 2.72 (d, 6H), 2.91-3.18 (m, 4H), 3.34-3.43 (m, 1H), 4.04-4.25 (m, 4H), 4.42-4.57 (m, 2H), 4.69-4.81 (m, 1H), 7.28 (m, 3H), 7.40 (d, 2H), 7.80 (m, 7H), 7.98 (d, 2H), 8.26 (d, 1H), 8.54 (t, 1H), 10.38-10.61 (m, 2H), 15.01-15.23 (m, 1H).

LC-MS (Method 1): Rₜ=0.57 min; MS (ESIpos): m/z=892.6 [M+H—HCl]⁺.

### Example 115

### 3-{5-[4-({(2S)-2-({[trans-4-(Aminomethyl)cyclohexyl]carbonyl}amino)-3-[2′-methyl-4′-(octahydrocyclopenta[b]pyrrol-4-ylcarbamoyl)biphenyl-4-yl]propanoyl}amino)phenyl]-4H-1,2,4-triazol-3-yl}-2,2,3,3-tetrafluoropropanoic acid hydrochloride

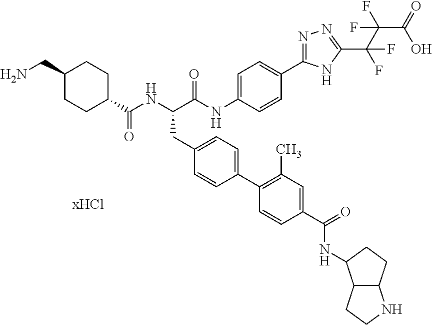 [SMILES: Cc1cc(C(=O)NC2CCC3NCCC32)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

To a solution of 26 mg (26 μmol) of 3-{5-[4-({(2S)-2-({[trans-4-(aminomethyl)cyclohexyl]-carbonyl}amino)-3-[2′-methyl-4′-(octahydrocyclopenta[b]pyrrol-4-ylcarbamoyl)biphenyl-4-yl]propanoyl}amino)phenyl]-4H-1,2,4-triazol-3-yl}-2,2,3,3-tetrafluoropropanoic acid in 1 ml of dioxane were added 96 μl (0.38 mmol) of 4M hydrogen chloride in dioxane. The mixture was stirred at RT for 18 h. Acetonitrile was added and the solid obtained was filtered, washed with acetonitrile and dried under high vacuum. 24 mg (99% of theory) of the title compound were obtained.

¹H NMR (400 MHz, DMSO-d₆): δ=ppm 0.79-1.02 (m, 2H), 1.12-1.32 (m, 3H), 1.41-1.54 (m, 1H), 1.76 (m, 9H), 2.09-2.20 (m, 1H), 2.23 (s, 3H), 2.59-2.69 (m, 2H), 2.70-2.81 (m, 1H), 2.91-3.19 (m, 4H), 3.97-4.11 (m, 1H), 4.16-4.32 (m, 1H), 4.68-4.83 (m, 1H), 7.26 (m, 3H), 7.39 (d, 2H), 7.78 (m, 6H), 7.97 (d, 2H), 8.25 (d, 1H), 8.51 (d, 1H), 8.84 (br. s, 1H), 9.19 (br. s, 1H), 10.44 (s, 1H), 14.98-15.19 (m, 1H).

LC-MS (Method 1): Rₜ=0.59 min; MS (ESIpos): m/z=833.3 [M+H—HCl]⁺.

### Example 116

### 3-{5-[4-({(2S)-2-({[trans-4-(Aminomethyl)cyclohexyl]carbonyl}amino)-3-[4′-(isopropylcarbamoyl)-2′-methylbiphenyl-4-yl]propanoyl}amino)phenyl]-4H-1,2,4-triazol-3-yl}-2,2,3,3-tetrafluoropropanoic acid hydrochloride

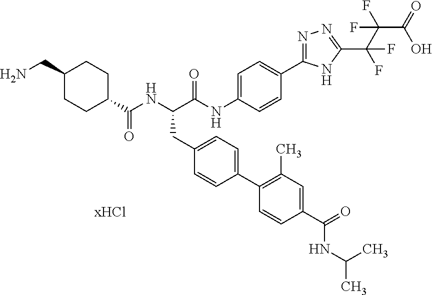 [SMILES: Cc1cc(C(=O)NC(C)C)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

To a solution of 23 mg (26 μmol) of 3-{5-[4-({(2S)-2-{[(trans-4-{[(tert-butoxycarbonyl)-amino]methyl}cyclohexyl)carbonyl]amino}-3-[4′-(isopropylcarbamoyl)-2′-methylbiphenyl-4-yl]propanoyl}amino)phenyl]-4H-1,2,4-triazol-3-yl}-2,2,3,3-tetrafluoropropanoic acid in 1 ml of dioxane were added 99 μl (0.48 mmol) of 4M hydrogen chloride in dioxane. The mixture was stirred at RT for 18 h. The solid obtained was filtered off, washed with acetonitrile and dried under high vacuum. 21 mg (81% of theory) of the title compound were obtained.

¹H NMR (400 MHz, DMSO-d₆): δ=ppm 0.75-1.02 (m, 2H), 1.17 (d, 6H), 1.22-1.33 (m, 1H), 1.40-1.61 (m, 2H), 1.65-1.84 (m, 3H), 2.09-2.19 (m, 1H), 2.23 (s, 3H), 2.60-2.68 (m, 2H), 2.75-2.81 (m, 1H), 2.90-3.01 (m, 1H), 3.10-3.20 (m, 1H), 3.36 (d, 1H), 4.04-4.16 (m, 1H), 4.71-4.80 (m, 1H), 7.26 (m, 3H), 7.39 (d, 2H), 7.64-7.83 (m, 7H), 7.97 (d, 2H), 8.19 (d, 1H), 8.28 (d, 1H), 10.51 (br. s, 1H), 15.16 (br. s, 1H).

LC-MS (Method 1): Rₜ=0.73 min; MS (ESIpos): m/z=766.4 [M+H—HCl]⁺.

### Example 117

### 4′-[(2S)-2-({[trans-4-(Aminomethyl)cyclohexyl]carbonyl}amino)-3-oxo-3-{[4-(2H-tetrazol-5-yl)phenyl]amino}propyl]-2,6-dimethoxy-N-(1-methylpiperidin-4-yl)biphenyl-4-carboxamide hydrochloride

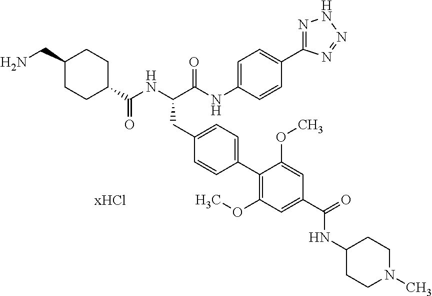 [SMILES: COc1cc(C(=O)NC2CCN(C)CC2)cc(OC)c1-c1ccc(C[C@H](NC(=O)[C@H]2CC[C@H](CN)CC2)C(=O)Nc2ccc(-c3nn[nH]n3)cc2)cc1]

tert-Butyl [(trans-4-{[(2S)-3-{2′,6′-dimethoxy-4′-[(1-methylpiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]-carbamate (60 mg, 0.07 mmol) was initially charged in 1.0 ml of dioxane, 0.36 ml (1.46 mmol) of 4M hydrogen chloride in dioxane were added and the mixture was stirred at RT for 1 h and left to stand overnight. The reaction mixture was concentrated, then admixed with 1.0 ml of 4M hydrogen chloride in dioxane and stirred at RT for 1 h and left to stand overnight. The reaction mixture was diluted with dioxane, and the solid present was filtered off and washed three times with diethyl ether. The residue was dried under high vacuum. 49 mg (81% of theory) of the title compound were obtained.

¹H NMR (400 MHz, DMSO-d₆): δ=ppm 10.52 (br. s., 1H), 10.35-10.22 (m, 1H), 8.60-8.51 (m, 1H), 8.31-8.22 (m, 1H), 8.02 (d, 2H), 7.88-7.72 (m, 4H), 7.31 (d, 2H), 7.22 (s, 2H), 7.12 (d, 2H), 4.78-4.68 (m, 1H), 4.12-3.98 (m, 1H), 3.70 (s, 6H), 3.17-3.03 (m, 3H), 2.99-2.86 (m, 1H), 2.80-2.60 (m, 5H), 2.23-2.10 (m, 1H), 2.09-1.84 (m, 4H), 1.83-1.69 (m, 3H), 1.68-1.57 (m, 1H), 1.56-1.42 (m, 1H), 1.35-1.17 (m, 2H), 1.02-0.85 (m, 2H).

LC-MS (Method 1): Rₜ=0.52 min; MS (ESIpos): m/z=724 [M+H—HCl]⁺.

### Example 118

### 4′-[(2S)-2-({[trans-4-(Aminomethyl)cyclohexyl]carbonyl}amino)-3-oxo-3-{[4-(2H-tetrazol-5-yl)phenyl]amino}propyl]-3,5-difluoro-N-(1-methylpiperidin-4-yl)biphenyl-4-carboxamide hydrochloride

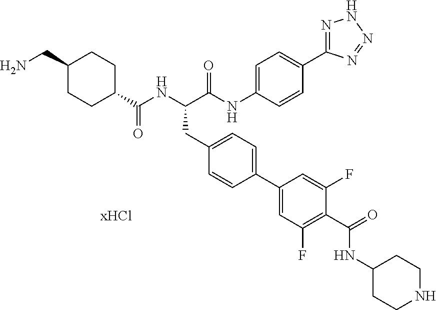 [SMILES: NC[C@H]1CC[C@H](C(=O)N[C@@H](Cc2ccc(-c3cc(F)c(C(=O)NC4CCNCC4)c(F)c3)cc2)C(=O)Nc2ccc(-c3nn[nH]n3)cc2)CC1]

tert-Butyl 4-[({4′-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]-3,5-difluorobiphenyl-4-yl}carbonyl)amino]piperidine-1-carboxylate (42 mg, 0.05 mmol) was initially charged in 0.5 ml of dioxane, 0.48 ml (1.91 mmol) of 4M hydrogen chloride in dioxane were added and the mixture was stirred at RT for 1 h. The reaction mixture was diluted with dioxane, and the solid present was filtered off, washed three times with diethyl ether and dried under high vacuum. 30 mg (75% of theory) of the title compound were obtained.

¹H NMR (400 MHz, DMSO-d₆): δ=ppm 10.57 (br. s., 1H), 8.91 (d, 1H), 8.80-8.70 (m, 1H), 8.69-8.59 (m, 1H), 8.28 (d, 1H), 8.02 (d, 2H), 7.87-7.70 (m, 8H), 7.54 (d, 2H), 7.44 (d, 2H), 4.77-4.68 (m, 1H), 4.10-4.00 (m, 1H), 3.29-3.24 (m, 2H), 3.17-3.10 (m, 1H), 3.08-2.99 (m, 2H), 2.98-2.91 (m, 1H), 2.69-2.59 (m, 2H), 2.19-2.11 (m, 1H), 2.05-1.94 (m, 2H), 1.82-1.64 (m, 4H), 1.63-1.57 (m, 1H), 1.52-1.42 (m, 1H), 1.32-1.14 (m, 2H), 0.98-0.85 (m, 2H).

LC-MS (Method 1): Rₜ=0.53 min; MS (ESIpos): m/z=686 [M+H—HCl]⁺.

### Example 119

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-ethoxy-N-(piperidin-4-yl)biphenyl-4-carboxamide hydrochloride

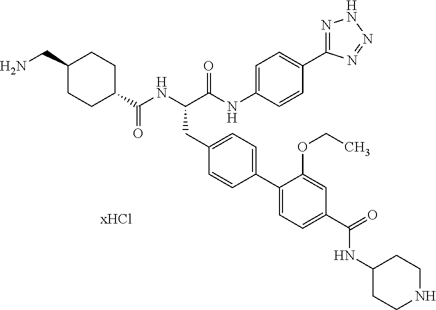 [SMILES: CCOc1cc(C(=O)NC2CCNCC2)ccc1-c1ccc(C[C@H](NC(=O)[C@H]2CC[C@H](CN)CC2)C(=O)Nc2ccc(-c3nn[nH]n3)cc2)cc1]

tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2-ethoxybiphenyl-4-yl}carbonyl)-amino]piperidine-1-carboxylate (62 mg, 0.07 mmol) was initially charged in 1.0 ml of dioxane, 0.69 ml (2.77 mmol) of 4M hydrogen chloride in dioxane were added and the mixture was stirred at RT for 1 h. The reaction mixture was diluted with dioxane, and the solid present was filtered off, washed with diethyl ether and dried under high vacuum. 50 mg (85% of theory) of the title compound were obtained.

¹H NMR (400 MHz, DMSO-d₆): δ=ppm 16.80 (br. s., 1H), 10.51 (s, 1H), 8.75-8.53 (m, 2H), 8.49 (d, 1H), 8.30-8.23 (m, 1H), 8.01 (d, 2H), 7.86-7.68 (m, 5H), 7.57-7.43 (m, 4H), 7.41-7.31 (m, 3H), 4.77-4.68 (m, 1H), 4.15-4.02 (m, 3H), 3.16-2.87 (m, 4H), 2.69-2.59 (m, 2H), 2.23-2.11 (m, 1H), 2.04-1.93 (m, 2H), 1.84-1.69 (m, 5H), 1.69-1.59 (m, 1H), 1.55-1.41 (m, 1H), 1.35-1.15 (m, 5H), 1.01-0.85 (m, 2H).

LC-MS (Method 1): Rₜ=0.54 min; MS (ESIpos): m/z=694 [M+H—HCl]⁺.

### Example 120

### 4′-[(2S)-2-({[trans-4-(Aminomethyl)cyclohexyl]carbonyl}amino)-3-oxo-3-{[4-(2H-tetrazol-5-yl)phenyl]amino}propyl]-N-(piperidin-4-yl)-3-(trifluoromethyl)biphenyl-4-carboxamide hydrochloride

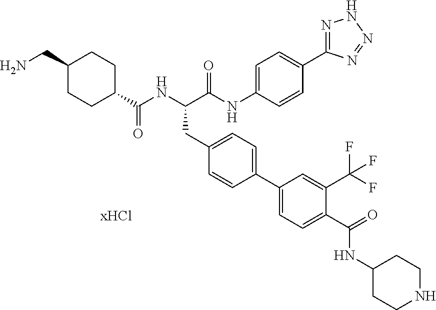 [SMILES: NC[C@H]1CC[C@H](C(=O)N[C@@H](Cc2ccc(-c3ccc(C(=O)NC4CCNCC4)c(C(F)(F)F)c3)cc2)C(=O)Nc2ccc(-c3nn[nH]n3)cc2)CC1]

tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-3-(trifluoromethyl)biphenyl-4-yl}carbonyl)amino]piperidine-1-carboxylate (131 mg, 0.14 mmol) was initially charged in 1.5 ml of dioxane, 1.43 ml (5.73 mmol) of 4M hydrogen chloride in dioxane were added and the mixture was stirred at RT for 1 h. The reaction mixture was diluted with dioxane, and the solid present was filtered off, washed with diethyl ether and dried under high vacuum. 112 mg (95% of theory) of the title compound were obtained.

¹H NMR (400 MHz, DMSO-d₆): δ=ppm 10.61 (br. s., 1H), 8.93-8.82 (m, 1H), 8.81-8.66 (m, 2H), 8.29 (d, 1H), 8.07-7.94 (m, 4H), 7.93-7.78 (m, 5H), 7.71 (d, 2H), 7.59 (d, 1H), 7.47 (d, 1H), 4.79-4.68 (m, 1H), 4.10-3.98 (m, 1H), 3.19-3.11 (m, 1H), 3.08-2.92 (m, 3H), 2.67-2.58 (m, 2H), 2.21-2.10 (m, 1H), 2.05-1.93 (m, 2H), 1.83-1.55 (m, 6H), 1.54-1.42 (m, 1H), 1.34-1.13 (m, 2H), 1.00-0.84 (m, 2H).

LC-MS (Method 1): Rₜ=0.53 min; MS (ESIpos): m/z=718 [M+H—HCl]⁺.

### Example 121

### 4′-[(2S)-2-({[trans-4-(Aminomethyl)cyclohexyl]carbonyl}amino)-3-oxo-3-{[4-(2H-tetrazol-5-yl)phenyl]amino}propyl]-3-fluoro-2-methoxy-N-(piperidin-4-yl)biphenyl-4-carboxamide hydrochloride

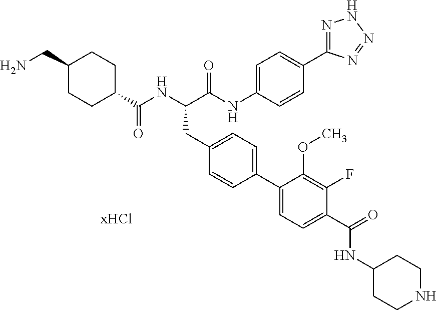 [SMILES: COc1c(-c2ccc(C[C@H](NC(=O)[C@H]3CC[C@H](CN)CC3)C(=O)Nc3ccc(-c4nn[nH]n4)cc3)cc2)ccc(C(=O)NC2CCNCC2)c1F]

tert-Butyl 4-[({4′-[(2S)-2-{[(trans-4-{[(tert-butoxycarbonyl)amino]methyl}cyclohexyl)carbonyl]-amino}-3-oxo-3-({[4-(2H-tetrazol-5-yl)phenyl]amino}propyl]-3-fluoro-2-methoxybiphenyl-4-yl}carbonyl)amino]piperidine-1-carboxylate (30 mg, 0.03 mmol) was initially charged in 0.8 ml of dioxane, 0.17 ml of 4M hydrogen chloride in dioxane was added and the mixture was stirred at RT overnight. The suspension obtained was filtered, and the solid was washed with water and dried under high vacuum. 24 mg (83% of theory) of the title compound were obtained.

¹H NMR (400 MHz, DMSO-d₆): δ=ppm 16.84-16.68 (m, 1H), 10.54-10.46 (m, 1H), 8.73-8.64 (m, 1H), 8.58-8.51 (m, 1H), 8.30-8.23 (m, 1H), 8.05-7.96 (m, 1H), 7.86-7.67 (m, 4H), 7.47-7.36 (m, 3H), 7.33-7.25 (m, 1H), 7.23-7.16 (m, 1H), 4.79-4.70 (m, 1H), 4.10-3.99 (m, 1H), 3.61-3.55 (m, 3H), 3.17-3.09 (m, 1H), 3.08-2.88 (m, 2H), 2.69-2.59 (m, 2H), 2.21-2.09 (m, 1H), 2.04-1.95 (m, 1H), 1.81-1.66 (m, 4H), 1.65-1.57 (m, 1H), 1.53-1.42 (m, 1H), 1.32-1.12 (m, 2H), 0.99-0.84 (m, 2H).

LC-MS (Method 1): Rₜ=0.51 min; MS (ESIpos): m/z=698 [M+H—HCl]⁺.

### Example 122

### 4′-[(2S)-2-({[trans-4-(Aminomethyl)cyclohexyl]carbonyl}amino)-3-oxo-3-{[4-(2H-tetrazol-5-yl)phenyl]amino}propyl]-3-chloro-N-(piperidin-4-yl)biphenyl-4-carboxamide hydrochloride

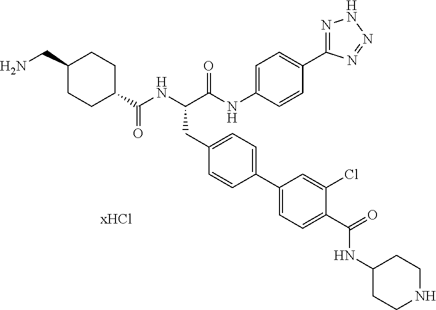 [SMILES: NC[C@H]1CC[C@H](C(=O)N[C@@H](Cc2ccc(-c3ccc(C(=O)NC4CCNCC4)c(Cl)c3)cc2)C(=O)Nc2ccc(-c3nn[nH]n3)cc2)CC1]

tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-3-chlorobiphenyl-4-yl}carbonyl)-amino]piperidine-1-carboxylate (110 mg, 0.13 mmol) was initially charged in 1.0 ml of dioxane, 0.62 ml of 4M hydrogen chloride in dioxane were added and the mixture was stirred in an ultrasound bath at RT for 45 min. The reaction mixture was diluted with 5 ml of dioxane and filtered. The filter residue was washed twice with 2 ml each time of dioxane and three times with 2 ml each time of diethyl ether, and dried under high vacuum. 94 mg (94% of theory) of the title compound were obtained.

¹H NMR (400 MHz, DMSO-d₆): δ=ppm 16.86 (br. s., 1H), 10.58 (br. s., 1H), 8.89-8.77 (m, 1H), 8.76-8.59 (m, 2H), 8.34-8.22 (m, 1H), 8.06-8.00 (m, 2H), 7.92-7.74 (m, 6H), 7.72-7.61 (m, 3H), 7.53-7.39 (m, 3H), 4.78-4.67 (m, 1H), 4.11-3.98 (m, 1H), 3.30-3.21 (m, 2H), 3.18-3.09 (m, 1H), 3.09-2.90 (m, 3H), 2.70-2.59 (m, 2H), 2.22-2.10 (m, 1H), 2.05-1.95 (m, 2H), 1.85-1.65 (m, 5H), 1.65-1.56 (m, 1H), 1.55-1.43 (m, 1H), 1.33-1.12 (m, 2H), 1.02-0.84 (m, 2H).

LC-MS (Method 1): Rₜ=0.51 min; MS (ESIpos): m/z=684 [M+H—HCl]⁺.

### Example 123

### 4′-[(2S)-2-({[trans-4-(Aminomethyl)cyclohexyl]carbonyl}amino)-3-oxo-3-{[4-(2H-tetrazol-5-yl)phenyl]amino}propyl]-2,3-dimethyl-N-(piperidin-4-yl)biphenyl-4-carboxamide hydrochloride

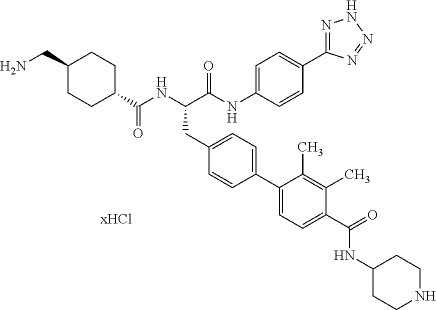 [SMILES: Cc1c(C(=O)NC2CCNCC2)ccc(-c2ccc(C[C@H](NC(=O)[C@H]3CC[C@H](CN)CC3)C(=O)Nc3ccc(-c4nn[nH]n4)cc3)cc2)c1C]

tert-Butyl 4-[({4′-[(2S)-2-{[(trans-4-{[(tert-butoxycarbonyl)amino]methyl}cyclohexyl)carbonyl]-amino}-3-oxo-3-{[4-(2H-tetrazol-5-yl)phenyl]amino}propyl]-2,3-dimethylbiphenyl-4-yl}-carbonyl)amino]piperidine-1-carboxylate (72 mg, 0.08 mmol) was initially charged in 2.0 ml of dioxane, 0.41 ml of 4M hydrogen chloride in dioxane was added and the mixture was left to stand at RT overnight. The reaction mixture was concentrated, and the residue was stirred with diethyl ether and filtered. The filter residue was dried under high vacuum and separated by means of preparative HPLC (eluent: acetonitrile/water with 0.1% trifluoroacetic acid (gradient)). The product-containing fractions were concentrated and lyophilized. The crude product thus obtained was taken up in 1.0 ml of dioxane, admixed with 0.5 ml of 4M hydrogen chloride in dioxane and stirred at RT for 15 min. The reaction mixture was concentrated, and the residue was admixed with dioxane and concentrated again. The residue was stirred with diethyl ether and filtered off, and the filter residue was dried under high vacuum. 20 mg (30% of theory) of the title compound were obtained.

¹H NMR (400 MHz, DMSO-d₆): δ=ppm 16.80 (br. s., 1H), 10.50 (s, 1H), 8.80-8.67 (m, 1H), 8.60-8.46 (m, 1H), 8.42 (d, 1H), 8.26 (d, 1H), 8.01 (d, 2H), 7.89-7.68 (m, 5H), 7.38 (d, 2H), 7.19-7.08 (m, 3H), 7.01 (d, 1H), 4.81-4.70 (m, 1H), 4.10-3.98 (m, 1H), 3.29-3.20 (m, 2H), 3.18-3.08 (m, 1H), 3.07-2.90 (m, 3H), 2.69-2.59 (m, 2H), 2.24 (s, 3H), 2.20-2.11 (m, 1H), 2.07 (s, 3H), 2.04-1.95 (m, 2H), 1.82-1.63 (m, 5H), 1.62-1.54 (m, 1H), 1.53-1.41 (m, 1H), 1.35-1.13 (m, 2H), 1.01-0.83 (m, 2H).

LC-MS (Method 1): Rₜ=0.53 min; MS (ESIpos): m/z=678 [M+H—HCl]⁺.

### Example 124

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-3-methyl-N-(piperidin-4-yl)biphenyl-4-carboxamide hydrochloride

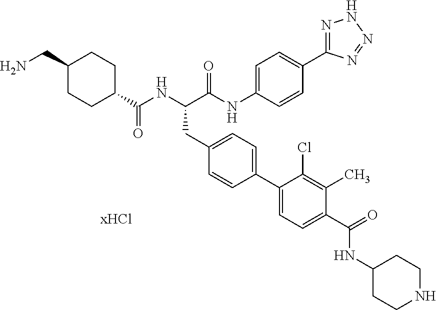 [SMILES: Cc1c(C(=O)NC2CCNCC2)ccc(-c2ccc(C[C@H](NC(=O)[C@H]3CC[C@H](CN)CC3)C(=O)Nc3ccc(-c4nn[nH]n4)cc3)cc2)c1Cl]

tert-Butyl 4-[({4′-[(2S)-2-{[(trans-4-{[(tert-butoxycarbonyl)amino]methyl}cyclohexyl)carbonyl]-amino}-3-oxo-3-({[4-(2H-tetrazol-5-yl)phenyl]amino}propyl]-2-chloro-3-methylbiphenyl-4-yl}carbonyl)amino]piperidine-1-carboxylate (98 mg, 0.11 mmol) was initially charged in 2.0 ml of dioxane, 0.54 ml of 4M hydrogen chloride in dioxane was added and the mixture was left to stand at RT overnight. The reaction mixture was concentrated, and the residue was stirred with diethyl ether and filtered. The filter residue was dried under high vacuum and separated by means of preparative HPLC (eluent: acetonitrile/water with 0.1% trifluoroacetic acid (gradient)). The product-containing fractions were concentrated and lyophilized. The crude product thus obtained was taken up in 1.0 ml of dioxane, admixed with 0.5 ml of 4M hydrogen chloride in dioxane and stirred at RT for 15 min. The reaction mixture was concentrated, and the residue was admixed with dioxane and concentrated again. The residue was stirred with diethyl ether and filtered off, and the filter residue was dried under high vacuum. 27 mg (29% of theory) of the title compound were obtained.

¹H NMR (400 MHz, DMSO-d₆): δ=ppm 10.50 (s, 1H), 8.74-8.65 (m, 1H), 8.61-8.43 (m, 2H), 8.26 (d, 1H), 8.01 (d, 2H), 7.86-7.67 (m, 5H), 7.40 (d, 2H), 7.33-7.25 (m, 3H), 7.21 (d, 1H), 4.81-4.70 (m, 1H), 4.11-3.98 (m, 1H), 3.29-3.22 (m, 2H), 3.19-3.10 (m, 1H), 3.09-2.90 (m, 3H), 2.69-2.59 (m, 2H), 2.38 (s, 3H), 2.21-2.10 (m, 1H), 2.05-1.96 (m, 2H), 1.83-1.53 (m, 6H), 1.53-1.40 (m, 1H), 1.35-1.13 (m, 2H), 1.01-0.84 (m, 2H).

LC-MS (Method 1): Rₜ=0.55 min; MS (ESIpos): m/z=698 [M+H—HCl]⁺.

### Example 125

### 4′-[(2S)-2-({[trans-4-(Aminomethyl)cyclohexyl]carbonyl}amino)-3-oxo-3-{[4-(2H-tetrazol-5-yl)phenyl]amino}propyl]-2-chloro-3-methyl-N-(1-methylpiperidin-4-yl)biphenyl-4-carboxamide hydrochloride

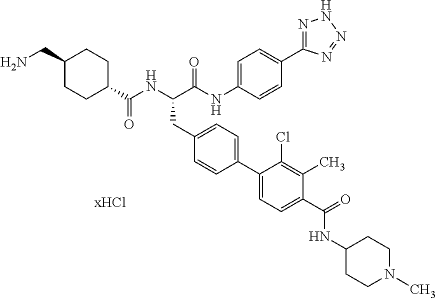 [SMILES: Cc1c(C(=O)NC2CCN(C)CC2)ccc(-c2ccc(C[C@H](NC(=O)[C@H]3CC[C@H](CN)CC3)C(=O)Nc3ccc(-c4nn[nH]n4)cc3)cc2)c1Cl]

tert-Butyl [(trans-4-({[(2S)-3-{2′-chloro-3′-methyl-4′-[(1-methylpiperidin-4-yl)carbamoyl]biphenyl-4-yl}-1-oxo-1-{[4-(2H-tetrazol-5-yl)phenyl]amino}propan-2-yl]carbamoyl}cyclohexyl)methyl]-carbamate (18 mg, 0.02 mmol) was initially charged in 1.0 ml of dioxane, 0.05 ml of 4M hydrogen chloride in dioxane was added and the mixture was stirred in an ultrasound bath at RT for 90 min. The reaction mixture was concentrated, and the residue was taken up in dioxane, admixed again with 4M hydrogen chloride in dioxane and stirred at RT for 3 h. The mixture was concentrated, and the residue was stirred in acetonitrile and filtered. The filter residue was dried under high vacuum. 9 mg (48% of theory) of the title compound were obtained.

¹H NMR (400 MHz, DMSO-d₆): δ=ppm 16.75 (br. s., 1H), 10.51 (s, 1H), 9.94 (br. s., 1H), 8.58 (d, 1H), 8.26 (d, 1H), 8.01 (d, 2H), 7.87-7.68 (m, 5H), 7.40 (d, 2H), 7.33-7.25 (m, 3H), 7.21 (d, 1H), 4.81-4.70 (m, 1H), 4.04-3.91 (m, 1H), 3.48-3.39 (m, 2H), 3.20-3.04 (m, 3H), 3.01-2.90 (m, 1H), 2.74 (s, 3H), 2.68-2.60 (m, 2H), 2.38 (s, 3H), 2.20-2.11 (m, 1H), 2.11-2.00 (m, 2H), 1.74 (br. s., 5H), 1.64-1.54 (m, 1H), 1.53-1.40 (m, 1H), 1.34-1.13 (m, 3H), 1.01-0.83 (m, 2H).

LC-MS (Method 1): Rₜ=0.55 min; MS (ESIpos): m/z=712 [M+H—HCl]⁺.

### Example 126

### 4′-{(2S)-2-({[trans-4-(Aminomethyl)cyclohexyl]carbonyl}amino)-3-[(4-{3-[3-(dimethylamino)-1,1,2,2-tetrafluoro-3-oxopropyl]-1H-1,2,4-triazol-5-yl}phenyl)amino]-3-oxopropyl}-2-methyl-N-(piperidin-4-yl)biphenyl-4-carboxamide hydrochloride

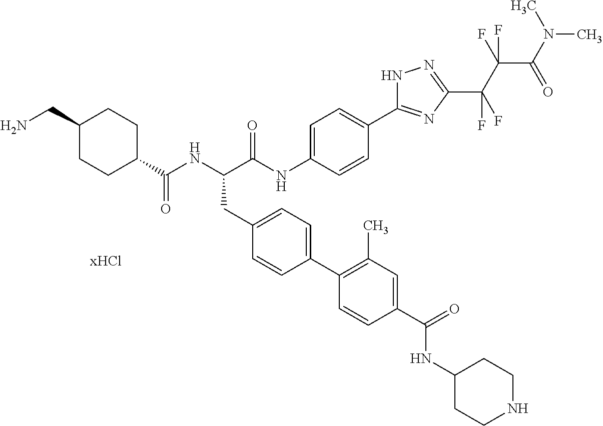 [SMILES: Cc1cc(C(=O)NC2CCNCC2)ccc1-c1ccc(C[C@H](NC(=O)[C@H]2CC[C@H](CN)CC2)C(=O)Nc2ccc(-c3nc(C(F)(F)C(F)(F)C(=O)N(C)C)n[nH]3)cc2)cc1]

To a solution of 83 mg (80 μmol) of tert-butyl 4-{[(4′-{(2S)-2-{[(trans-4-{[(tert-butoxycarbonyl)amino]methyl}cyclohexyl)carbonyl]amino}-3-[(4-{3-[3-(dimethylamino)-1,1,2,2-tetrafluoro-3-oxopropyl]-1H-1,2,4-triazol-5-yl}phenyl)amino]-3-oxopropyl}-2-methylbiphenyl-4-yl)carbonyl]amino}piperidine-1-carboxylate in 2 ml of dioxane were added 60 μl (0.24 mmol) of 4M hydrogen chloride in dioxane. The mixture was stirred at RT for 18 h. Acetonitrile was added and the solid obtained was filtered, washed with acetonitrile and dried under high vacuum. 59 mg (81% of theory) of the title compound were obtained.

¹H NMR (400 MHz, DMSO-d₆): δ=ppm 0.80-1.02 (m, 2H), 1.05-1.34 (m, 2H), 1.42-1.62 (m, 2H), 1.68-1.85 (m, 5H), 1.97 (m, 2H), 2.16 (t, 1H), 2.23 (s, 3H), 2.57-2.69 (m, 2H), 2.94 (s, 3H), 2.96-3.06 (m, 3H), 3.08-3.18 (m, 4H), 3.31 (d, 2H), 3.57 (s, 1H), 4.07 (m, 1H), 4.75 (m, 1H), 7.16-7.28 (m, 3H), 7.41 (d, 2H), 7.69-7.94 (m, 7H), 8.00 (d, 2H), 8.29 (d, 1H), 8.49 (d, 1H), 8.92 (br. s., 2H), 10.55 (br. s., 1H), 15.09 (br. s, 1H).

LC-MS (Method 1): Rₜ=0.63 min; MS (ESIpos): m/z=834.5 [M+H—HCl]⁺.

### Example 127

### 4′-[(2S)-2-({[trans-4-(Aminomethyl)cyclohexyl]carbonyl}amino)-3-({4-[3-(3-amino-1,1,2,2-tetrafluoro-3-oxopropyl)-1H-1,2,4-triazol-5-yl]phenyl}amino)-3-oxopropyl]-2-methyl-N-(piperidin-4-yl)biphenyl-4-carboxamide hydrochloride

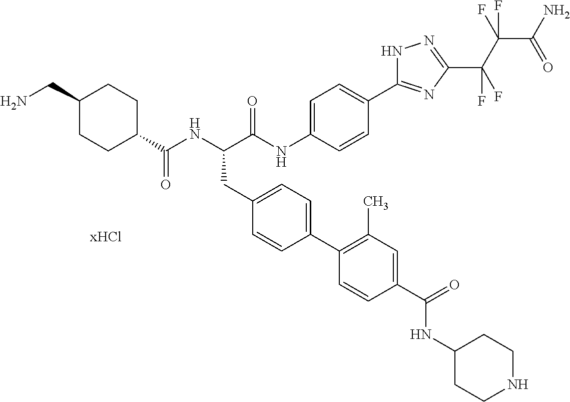 [SMILES: Cc1cc(C(=O)NC2CCNCC2)ccc1-c1ccc(C[C@H](NC(=O)[C@H]2CC[C@H](CN)CC2)C(=O)Nc2ccc(-c3nc(C(F)(F)C(F)(F)C(N)=O)n[nH]3)cc2)cc1]

To a solution of 22 mg (22 μmol) of tert-butyl 4-[({4′-[(2S)-3-({4-[3-(3-amino-1,1,2,2-tetrafluoro-3-oxopropyl)-1H-1,2,4-triazol-5-yl]phenyl}amino)-2-{[(trans-4-{[(tert-butoxycarbonyl)amino]-methyl}cyclohexyl)carbonyl]amino}-3-oxopropyl]-2-methylbiphenyl-4-yl}carbonyl)amino]-piperidine-1-carboxylate in 1 ml of dioxane were added 17 μl (0.07 mmol) of 4M hydrogen chloride in dioxane. The mixture was stirred at RT for 18 h. Acetonitrile was added and the solid obtained was filtered, washed with acetonitrile and dried under high vacuum. 14 mg (69% of theory) of the title compound were obtained.

¹H NMR (400 MHz, DMSO-d₆): δ=ppm 0.83-1.00 (m, 2H), 1.08-1.32 (m, 3H), 1.43-1.61 (m, 2H), 1.66-1.84 (m, 5H), 1.88-2.00 (m, 2H), 2.08-2.20 (m, 1H), 2.23 (s, 3H), 2.59-2.69 (m, 2H), 2.88-3.18 (m, 4H), 3.99-4.18 (m, 1H), 4.65-4.86 (m, 1H), 7.18-7.32 (m, 3H), 7.40 (d, 2H), 7.68-7.89 (m, 7H), 7.99 (d, 2H), 8.30 (d, 2H), 8.50 (d, 2H), 8.70-8.93 (m, 2H), 10.52 (s, 1H), 15.14 (br. s, 1H).

LC-MS (Method 1): Rₜ=0.57 min; MS (ESIneg): m/z=804.3 [M−H—HCl]⁻.

### Example 128

### 4′-{(2S)-2-({[trans-4-(Aminomethyl)cyclohexyl]carbonyl}amino)-3-oxo-3-[(4-{3-[1,1,2,2-tetrafluoro-3-(methylamino)-3-oxopropyl]-1H-1,2,4-triazol-5-yl}phenyl)amino]propyl}-2-methyl-N-(piperidin-4-yl)biphenyl-4-carboxamide hydrochloride

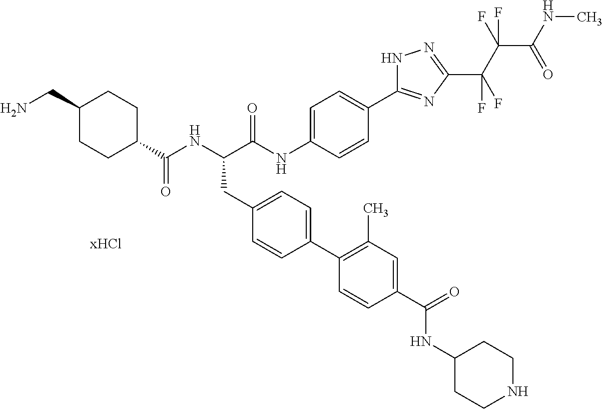 [SMILES: CNC(=O)C(F)(F)C(F)(F)c1n[nH]c(-c2ccc(NC(=O)[C@H](Cc3ccc(-c4ccc(C(=O)NC5CCNCC5)cc4C)cc3)NC(=O)[C@H]3CC[C@H](CN)CC3)cc2)n1]

To a solution of 41 mg (40 μmol) of tert-butyl 4-{[(4′-{(2S)-2-{[(trans-4-{[(tert-butoxycarbonyl)amino]methyl}cyclohexyl)carbonyl]amino}-3-oxo-3-[(4-{3-[1,2,2-tetrafluoro-3-(methylamino)-3-oxopropyl]-1H-1,2,4-triazol-5-yl}phenyl)amino]propyl}-2-methylbiphenyl-4-yl)carbonyl]amino}piperidine-1-carboxylate in 1 ml of dioxane were added 30 μl (0.12 mmol) of 4M hydrogen chloride in dioxane. The mixture was stirred at RT for 18 h. Acetonitrile was added and the solid obtained was filtered, washed with acetonitrile and dried under high vacuum. 22 mg (54% of theory) of the title compound were obtained.

¹H NMR (400 MHz, DMSO-d₆): δ=ppm 0.82-1.04 (m, 2H), 1.10-1.33 (m, 2H), 1.43-1.63 (m, 2H), 1.67-1.86 (m, 5H), 1.89-2.01 (m, 2H), 2.10-2.21 (m, 1H), 2.23 (s, 3H), 2.58-2.67 (m, 2H), 2.68-2.73 (m, 4H), 2.90-3.07 (m, 3H), 3.09-3.18 (m, 1H), 3.25-3.35 (m, 2H), 4.00-4.14 (m, 1H), 4.68-4.81 (m, 1H), 6.73 (d, 1H), 7.19-7.30 (m, 3H), 7.40 (d, 2H), 7.67-7.76 (m, 2H), 7.78-7.94 (m, 6H), 7.99 (d, 2H), 8.28 (d, 1H), 8.48 (d, 1H), 8.74-8.90 (m, 2H), 8.97-9.09 (m, 1H), 10.48-10.59 (m, 1H), 15.05-15.24 (m, 1H).

LC-MS (Method 1): Rₜ=0.59 min; MS (ESIneg): m/z=818.3 [M−H—HCl]⁻.

### Example 129

### Methyl 3-{5-[4-({(2S)-2-({[trans-4-(aminomethyl)cyclohexyl]carbonyl}amino)-3-[2′-methyl-4′-(piperidin-4-ylcarbamoyl)biphenyl-4-yl]propanoyl}amino)phenyl]-1H-1,2,4-triazol-3-yl}-2,2,3,3-tetrafluoropropanoate hydrochloride

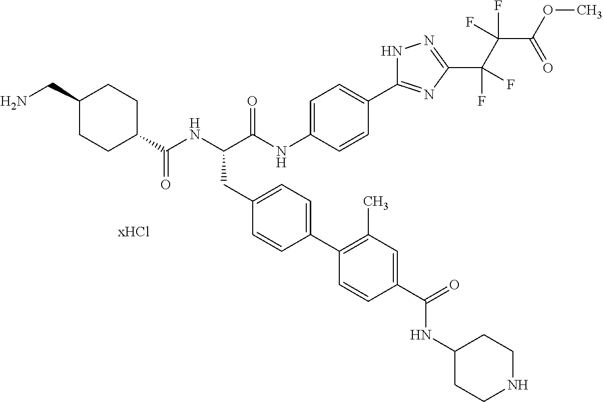 [SMILES: COC(=O)C(F)(F)C(F)(F)c1n[nH]c(-c2ccc(NC(=O)[C@H](Cc3ccc(-c4ccc(C(=O)NC5CCNCC5)cc4C)cc3)NC(=O)[C@H]3CC[C@H](CN)CC3)cc2)n1]

To a solution of 37 mg (36 μmol) of tert-butyl 4-[({4′-[(2S)-2-{[(trans-4-{[(tert-butoxycarbonyl)amino]methyl}cyclohexyl)carbonyl]amino}-3-oxo-3-({4-[3-(1, 1,2,2-tetrafluoro-3-methoxy-3-oxopropyl)-1H-1,2,4-triazol-5-yl]phenyl}amino)propyl]-2-methylbiphenyl-4-yl}carbonyl)amino]piperidine-1-carboxylate in 1 ml of dioxane were added 91 μl (0.36 mmol) of 4M hydrogen chloride in dioxane. The mixture was then stirred at RT for another 18 h. The solid obtained was filtered off, washed with acetonitrile and dried under high vacuum. 31 mg (85% of theory) of the title compound were obtained.

¹H NMR (400 MHz, DMSO-d₆): δ=ppm 0.83-1.00 (m, 2H), 1.10-1.35 (m, 2H), 1.43-1.63 (m, 2H), 1.76 (d, 5H), 1.92-2.04 (m, 2H), 2.10-2.20 (m, 1H), 2.23 (s, 3H), 2.58-2.68 (m, 2H), 2.90-3.09 (m, 3H), 3.10-3.18 (m, 1H), 3.95 (s, 3H), 4.01-4.16 (m, 1H), 4.67-4.83 (m, 1H), 7.17-7.30 (m, 3H), 7.40 (d, 2H), 7.68-7.88 (m, 7H), 7.98 (d, 2H), 8.27 (d, 1H), 8.47 (d, 1H), 8.63-8.83 (m, 2H), 10.52 (s, 1H), 15.26 (br. s, 1H).

LC-MS (Method 1): Rₜ=0.65 min; MS (ESIneg): m/z=819.5 [M−H—HCl]⁻.

### Example 130

### 3-[5-(4-{[(2S)-2-({[trans-4-(Aminomethyl)cyclohexyl]carbonyl}amino)-3-{2′-methyl-4′-[(1-methylpiperidin-4-yl)carbamoyl]biphenyl-4-yl}propanoyl]amino}phenyl)-1H-1,2,4-triazol-3-yl]-2,2,3,3-tetrafluoropropanoic acid

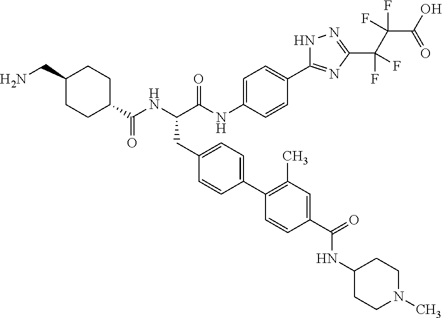 [SMILES: Cc1cc(C(=O)NC2CCN(C)CC2)ccc1-c1ccc(C[C@H](NC(=O)[C@H]2CC[C@H](CN)CC2)C(=O)Nc2ccc(-c3nc(C(F)(F)C(F)(F)C(=O)O)n[nH]3)cc2)cc1]

To a suspension of 2 g (2.25 μmol) of 3-[5-(4-{[(2S)-2-({[trans-4-(aminomethyl)cyclohexyl]-carbonyl}amino)-3-{2′-methyl-4′-[(1-methylpiperidin-4-yl)carbamoyl]biphenyl-4-yl}propanoyl]-amino}phenyl)-1H-1,2,4-triazol-3-yl]-2,2,3,3-tetrafluoropropanoic acid hydrochloride in 150 ml of water were added, in accordance with the chloride content of the reactant, 378.6 mg (0.5 mmol) of sodium hydrogencarbonate. The mixture was stirred at RT for 18 h. The solid obtained was repeatedly centrifuged and washed with water. The residue was dried under reduced pressure. 1.85 g (85% of theory) of the title compound were obtained.

¹H NMR (400 MHz, DMSO-d₆): δ=ppm 0.77-1.02 (m, 2H), 1.17-1.35 (m, 2H), 1.39-1.52 (m, 1H), 1.56-1.88 (m, 8H), 2.14 (t, 1H), 2.21 (s, 3H), 2.28-2.34 (m, 2H), 2.62-2.69 (m, 3H), 2.88-2.98 (m, 2H), 3.06-3.17 (m, 2H), 3.67-3.88 (m, 1H), 4.68-4.81 (m, 1H), 7.10-7.28 (m, 3H), 7.39 (d, 2H), 7.51-7.79 (m, 6H), 7.94 (d, 2H), 8.15-8.33 (m, 2H), 10.32 (s, 1H).

LC-MS (Method 1): Rₜ=0.57 min; MS (ESIpos): m/z=821.3 [M+H]⁺.

### Example 131

### 3-[5-(4-{[2-({[trans-4-(Aminomethyl)cyclohexyl]carbonyl}amino)-3-{4′-[(trans-4-hydroxycyclohexyl)carbamoyl]-2′-methylbiphenyl-4-yl}propanoyl]amino}phenyl)-4H-1,2,4-triazol-3-yl]-2,2,3,3-tetrafluoropropanoic acid (enantiomer mixture)

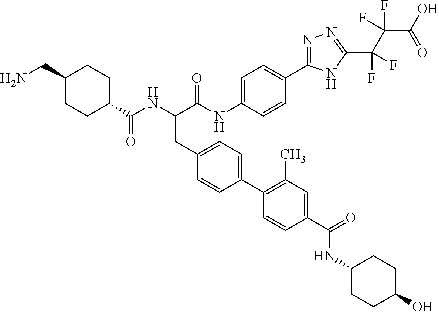 [SMILES: Cc1cc(C(=O)N[C@H]2CC[C@H](O)CC2)ccc1-c1ccc(CC(NC(=O)[C@H]2CC[C@H](CN)CC2)C(=O)Nc2ccc(-c3nnc(C(F)(F)C(F)(F)C(=O)O)[nH]3)cc2)cc1]

To a suspension of 530 mg (0.62 μmol) of 3-[5-(4-{[(2S)-2-({[trans-4-(aminomethyl)-cyclohexyl]carbonyl}amino)-3-{4′-[(trans-4-hydroxycyclohexyl)carbamoyl]-2′-methylbiphenyl-4-yl}propanoyl]amino}phenyl)-1H-1,2,4-triazol-3-yl]-2,2,3,3-tetrafluoropropanoic acid hydrochloride in 100 ml of water were added, in accordance with the chloride content of the reactant, 51.9 mg (0.62 mmol) of sodium hydrogencarbonate. The mixture was stirred at RT for 18 h. The solid obtained was repeatedly centrifuged and washed with water. The residue was dried under reduced pressure. 385 mg (72% of theory) of the title compound were obtained. It was established by analytical HPLC on a chiral column that the product was an enantiomer mixture.

¹H NMR (400 MHz, DMSO-d₆): δ=ppm 0.82-1.00 (m, 2H), 1.11-1.50 (m, 7H), 1.53-1.62 (m, 1H), 1.65-1.91 (m, 7H), 2.09-2.19 (m, 1H), 2.21 (s, 3H), 2.59-2.69 (m, 2H), 2.88-3.00 (m, 1H), 3.07-3.18 (m, 1H), 3.66-3.80 (m, 1H), 4.54 (d, 1H), 4.68-4.80 (m, 1H), 7.16-7.29 (m, 3H), 7.37 (d, 2H), 7.56-7.80 (m, 7H), 7.94 (d, 2H), 8.08-8.26 (m, 2H), 10.38 (s, 1H), 14.64 (br. s, 1H).

LC-MS (Method 1): Rₜ=0.64 min; MS (ESIpos): m/z=822.6 [M+H]⁺.

### Example 132

### 3-{5-[4-({(2S)-2-({[trans-4-(Aminomethyl)cyclohexyl]carbonyl}amino)-3-[4′-{[4-(dimethyl-amino)cyclohexyl]carbamoyl}-2′-(trifluoromethyl)biphenyl-4-yl]propanoyl}amino)phenyl]-1H-1,2,4-triazol-3-yl}-2,2,3,3-tetrafluoropropanoic acid hydrochloride

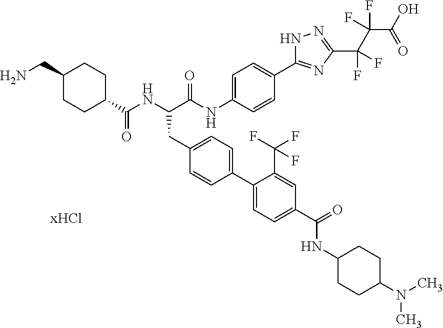

A solution of 30 mg (0.03 mmol) of 3-{5-[4-({(2S)-2-{[(trans-4-{[(tert-butoxycarbonyl)amino]-methyl}cyclohexyl)carbonyl]amino}-3-[4′-{[4-(dimethylamino)cyclohexyl]carbamoyl}-2′-(trifluoromethyl)biphenyl-4-yl]propanoyl}amino)phenyl]-1H-1,2,4-triazol-3-yl}-2,2,3,3-tetrafluoropropanoic acid in 1.5 ml of dichloromethane was admixed with 0.04 ml (0.15 mmol) of 4M hydrogen chloride in 1,4-dioxane and stirred at RT overnight. The reaction mixture was concentrated and purified by chromatography via HPLC (Method 7). 9 mg (31% of theory) of the title compound were obtained.

¹H NMR (300 MHz, DMSO-d₆): δ=ppm 0.80-1.00 (m, 2H), 1.10-1.48 (m, 8H), 1.52-1.64 (m, 1H), 1.67-1.80 (m, 3H), 1.80-1.98 (m, 4H), 2.21 (s, 6H), 2.58-2.65 (m, 2H), 2.89-3.01 (m, 1H), 3.08-3.13 (m, 1H), 3.68-3.84 (m, 1H), 4.66-4.82 (m, 1H), 7.17-7.27 (m, 2H), 7.34-7.43 (m, 3H), 7.74 (d, 2H), 7.93 (d, 2H), 8.10-8.16 (m, 1H), 8.19-8.29 (m, 3H), 8.51-8.60 (m, 1H), 10.27-10.41 (m, 1H).

LC-MS (Method 4): Rₜ=0.73 min; MS (ESIpos): m/z=903.6 [M+H—HCl]⁺

### Example 133

### 4′-{(2S)-2-({[trans-4-(Aminomethyl)cyclohexyl]carbonyl}amino)-3-oxo-3-[(2-oxo-2,3-dihydro-1H-benzimidazol-5-yl)amino]propyl}-2-methyl-N-[(2S)-1,1,1-trifluoropropan-2-yl]biphenyl-4-carboxamide

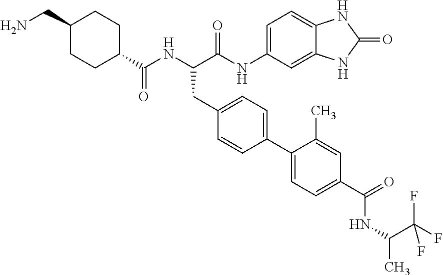

A solution of 126 mg (0.16 mmol) of tert-butyl {[trans-4-({(2S)-3-{2′-methyl-4′-([(2S)-1,1,1-trifluoropropan-2-yl]carbamoyl}biphenyl-4-yl)-1-oxo-1-[(2-oxo-2,3-dihydro-1H-benzimidazol-5-yl)amino]propan-2-yl}carbamoyl)cyclohexyl]methyl}carbamate in 3 ml of dichloromethane was admixed with 0.16 ml (0.66 mmol) of 4M hydrogen chloride in 1,4-dioxane and stirred at RT overnight. The reaction mixture was admixed with acetonitrile, and the precipitate was filtered off with suction and purified by chromatography via HPLC (Method 11). 17 mg (15% of theory) of the title compound were obtained.

¹H NMR (400 MHz, DMSO-d₆): δ=ppm 0.71-0.87 (m, 2H), 1.04-1.31 (m, 4H), 1.36 (d, 3H), 1.48-1.59 (m, 1H), 1.63-1.78 (m, 3H), 2.05-2.15 (m, 1H), 2.23 (s, 3H), 2.33 (d, 2H), 2.86-2.95 (m, 1H), 3.04-3.11 (m, 1H), 4.63-4.74 (m, 1H), 4.79-4.93 (m, 1H), 6.82 (d, 1H), 6.97-7.03 (m, 1H), 7.25 (m, 4H), 7.36 (d, 2H), 7.41 (d, 1H), 7.71-7.77 (m, 1H), 7.80 (s, 1H), 8.03-8.09 (m, 1H), 8.78-8.84 (m, 1H), 9.92 (s, 1H).

LC-MS (Method 5): Rₜ=1.07 min; MS (ESIpos): m/z=666.0 [M+H]⁺

### Example 134

### 4′-{(2S)-2-({[trans-4-(Aminomethyl)cyclohexyl]carbonyl}amino)-3-[(4-fluoro-1H-indazol-6-yl)amino]-3-oxopropyl}-2-chloro-N-isopropylbiphenyl-4-carboxamide hydrochloride

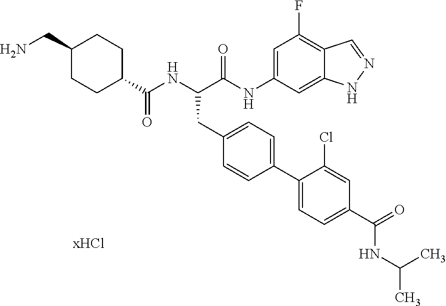

A solution of 112 mg (0.15 mmol) of tert-butyl {[trans-4-({(2S)-3-[2′-chloro-4′-(isopropylcarbamoyl)biphenyl-4-yl]-1-[(4-fluoro-1H-indazol-6-yl)amino]-1-oxopropan-2-yl}carbamoyl)cyclohexyl]methyl}carbamate in 6 ml of dichloromethane was admixed with 0.3 ml (1.2 mmol) of 4M hydrogen chloride in 1,4-dioxane and stirred at RT overnight. The reaction mixture was concentrated to dryness and purified by chromatography via HPLC (Method 8). 33 mg (31% of theory) of the title compound were obtained.

¹H NMR (300 MHz, DMSO-d₆): δ=ppm 0.79-0.98 (m, 2H), 1.17 (d, 6H), 1.22-1.46 (m, 2H), 1.51-1.63 (m, 1H), 1.64-1.83 (m, 3H), 2.07-2.21 (m, 1H), 2.91-3.01 (m, 1H), 3.09-3.16 (m, 1H), 4.03-4.16 (m, 1H), 4.65-4.78 (m, 1H), 6.99-7.09 (m, 1H), 7.38 (d, 5H), 7.82-7.90 (m, 2H), 7.96-8.02 (m, 1H), 8.07 (s, 1H), 8.28-8.34 (m, 1H), 8.34-8.40 (m, 1H), 8.40-8.44 (m, 1H), 10.42-10.48 (m, 1H).

LC-MS (Method 4): Rₜ=0.95 min; MS (ESIpos): m/z=633.5 [M+H—HCl]⁺

### Example 135

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-N-isopropyl-2-methylbiphenyl-4-carboxamide hydrochloride

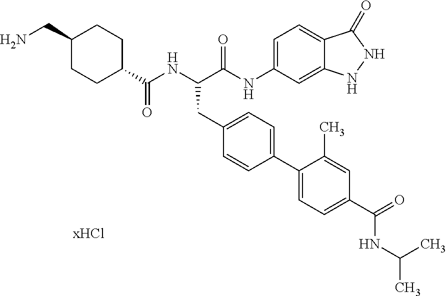

A solution of 100 mg (0.14 mmol) of tert-butyl {[trans-4-({(2S)-3-[4′-(isopropylcarbamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in 3 ml of dichloromethane was admixed with 0.14 ml (0.56 mmol) of 4M hydrogen chloride in 1,4-dioxane and stirred at RT overnight. The reaction mixture was admixed with acetonitrile, and the residue was filtered off with suction, washed with acetonitrile and dried under reduced pressure. 85 mg (90% of theory) of the title compound were obtained.

¹H NMR (400 MHz, DMSO-d₆): δ=ppm 0.86-1.00 (m, 2H), 1.18 (d, 6H), 1.21-1.36 (m, 2H), 1.40-1.52 (m, 1H), 1.54-1.63 (m, 1H), 1.68-1.83 (m, 3H), 2.10-2.19 (m, 1H), 2.23 (s, 3H), 2.60-2.66 (m, 2H), 2.95 (dd, 1H), 3.13 (dd, 1H), 4.07-4.16 (m, 1H), 4.71-4.82 (m, 1H), 6.99-7.05 (m, 1H), 7.21 (d, 1H), 7.27 (d, 2H), 7.36-7.43 (m, 2H), 7.51-7.57 (m, 1H), 7.68-7.73 (m, 1H), 7.77 (br. s., 3H), 7.86-7.89 (m, 1H), 8.18-8.27 (m, 2H), 10.29-10.32 (m, 1H), 11.06-11.27 (m, 1H).

LC-MS (Method 4): Rₜ=0.83 min; MS (ESIpos): m/z=611.5 [M+H—HCl]⁺

### Example 136

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-(1-cyclopropylethyl)-2-methylbiphenyl-4-carboxamide hydrochloride

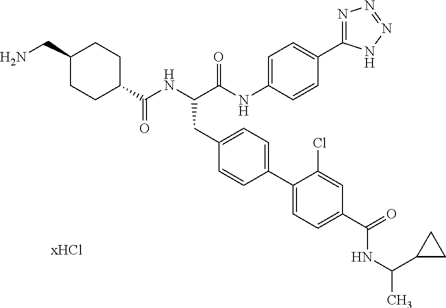

A solution of 140 mg (0.18 mmol) of tert-butyl [(trans-4-{[(2S)-3-{2′-chloro-4′-[(1-cyclopropylethyl)carbamoyl]biphenyl-4-yl}-1-oxo-1-{[4-(1H-tetrazol-5-yl)phenyl]amino}propan-2-yl]carbamoyl}cyclohexyl)methyl]carbamate in 6 ml of dichloromethane was admixed with 0.23 ml (0.9 mmol) of 4M hydrogen chloride in 1,4-dioxane and stirred at RT overnight. The reaction mixture was admixed with acetonitrile, and the residue was filtered off with suction, washed with acetonitrile and dried under reduced pressure. The residue was purified by chromatography via HPLC (Method 8). 36 mg (28% of theory) of the title compound were obtained.

¹H NMR (400 MHz, DMSO-d₆): δ=ppm 0.13-0.22 (m, 1H), 0.24-0.32 (m, 1H), 0.34-0.41 (m, 1H), 0.42-0.50 (m, 1H), 0.85-1.05 (m, 3H), 1.12-1.19 (m, 1H), 1.22 (d, 3H), 1.25-1.33 (m, 1H), 1.36-1.50 (m, 1H), 1.54-1.63 (m, 1H), 1.67-1.82 (m, 3H), 2.11-2.20 (m, 1H), 2.60-2.66 (m, 2H), 2.94 (dd, 1H), 3.13 (dd, 1H), 3.47-3.53 (m, 1H), 4.68-4.81 (m, 1H), 7.39 (m, 4H), 7.44-7.48 (m, 1H), 7.59 (d, 2H), 7.89 (m, 3H), 7.98-8.02 (m, 1H), 8.13-8.19 (m, 1H), 8.45-8.51 (m, 1H), 10.13 (s, 1H).

LC-MS (Method 4): Rₜ=0.99 min; MS (ESIpos): m/z=669.5 [M+H—HCl]⁺

### Example 137

### 4′-{(2S)-2-({[trans-4-(Aminomethyl)cyclohexyl]carbonyl}amino)-3-oxo-3-[(2-oxo-2,3-dihydro-1H-benzimidazol-5-yl)amino]propyl}-2-chloro-N-isopropylbiphenyl-4-carboxamide hydrochloride

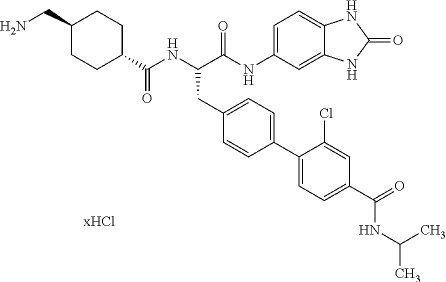

A solution of 154 mg (0.21 mmol) of tert-butyl {[trans-4-({(2S)-3-[2′-chloro-4′-(isopropyl-carbamoyl)biphenyl-4-yl]-1-oxo-1-[(2-oxo-2,3-dihydro-1H-benzimidazol-5-yl)amino]propan-2-yl}carbamoyl)cyclohexyl]methyl}carbamate in 7 ml of dichloromethane was admixed with 0.41 ml (1.7 mmol) of 4M hydrogen chloride in 1,4-dioxane and stirred at RT overnight. The reaction mixture was concentrated to dryness and purified by chromatography via HPLC (Method 8). 70 mg (49% of theory) of the title compound were obtained.

¹H NMR (300 MHz, DMSO-d₆): δ=ppm 0.76-0.98 (m, 2H), 1.06-1.13 (m, 1H), 1.17 (d, 6H), 1.21-1.28 (m, 1H), 1.30-1.44 (m, 1H), 1.49-1.60 (m, 1H), 1.65-1.80 (m, 3H), 2.07-2.18 (m, 1H), 2.54 (d, 2H), 2.91 (dd, 1H), 3.09 (dd, 1H), 4.01-4.15 (m, 1H), 4.62-4.74 (m, 1H), 6.84 (d, 1H), 6.99-7.05 (m, 1H), 7.32-7.46 (m, 5H), 7.84-7.89 (m, 1H), 8.00 (d, 1H), 8.18-8.24 (m, 1H), 8.41 (s, 1H), 10.01-10.06 (m, 1H).

LC-MS (Method 4): Rₜ=0.82 min; MS (ESIpos): m/z=631.6 [M+H—HCl]⁺

### Example 138

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-cyclobutyl-2-methylbiphenyl-4-carboxamide hydrochloride

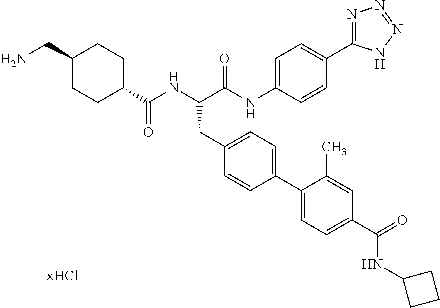

A solution of 32 mg (0.04 mmol) of tert-butyl [(trans-4-{[(2S)-3-[4′-(cyclobutylcarbamoyl)-2′-methylbiphenyl-4-yl]-1-oxo-1-{[4-(1H-tetrazol-5-yl)phenyl]amino}propan-2-yl]carbamoyl}-cyclohexyl)methyl]carbamate in 2 ml of dichloromethane was admixed with 0.07 ml (0.03 mmol) of 4M hydrogen chloride in 1,4-dioxane and stirred at RT overnight. The reaction mixture was concentrated to dryness and purified by chromatography via HPLC (Method 8). 10 mg (33% of theory) of the title compound were obtained.

¹H NMR (300 MHz, DMSO-d₆): δ=ppm 0.80-1.02 (m, 2H), 1.08-1.35 (m, 2H), 1.36-1.49 (m, 1H), 1.52-1.63 (m, 1H), 1.65-1.85 (m, 5H), 2.00-2.11 (m, 2H), 2.11-2.21 (m, 2H), 2.23 (s, 3H), 2.60-2.67 (m, 2H), 2.93 (dd, 1H), 3.12 (dd, 1H), 4.35-4.49 (m, 1H), 4.67-4.81 (m, 1H), 7.21-7.29 (m, 3H), 7.34-7.42 (m, 2H), 7.55-7.62 (m, 2H), 7.65-7.71 (m, 1H), 7.72-7.77 (m, 1H), 7.85-7.93 (m, 2H), 8.13-8.17 (m, 1H), 8.54-8.59 (m, 1H), 10.05-10.13 (m, 1H).

LC-MS (Method 4): Rₜ=0.93 min; MS (ESIpos): m/z=635.5 [M+H—HCl]⁺

### Example 139

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-(1-hydroxypropan-2-yl)-2-methylbiphenyl-4-carboxamide hydrochloride

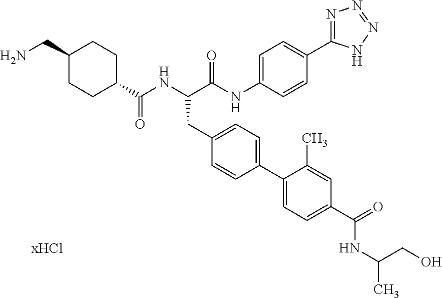

A solution of 30 mg (0.04 mmol) of tert-butyl [(trans-4-{[(2S)-3-{4′-[(1-hydroxypropan-2-yl)carbamoyl]-2′-methylbiphenyl-4-yl}-1-oxo-1-{[4-(1H-tetrazol-5-yl)phenyl]amino}propan-2-yl]carbamoyl}cyclohexyl)methyl]carbamate in 2 ml of dichloromethane was admixed with 0.05 ml (0.02 mmol) of 4M hydrogen chloride in 1,4-dioxane and stirred at RT overnight. The reaction mixture was concentrated to dryness and purified by chromatography via HPLC (Method 7). 3 mg (9% of theory) of the title compound were obtained.

¹H NMR (300 MHz, DMSO-d₆): δ=ppm 0.80-1.01 (m, 2H), 1.13 (d, 3H), 1.18-1.36 (m, 2H), 1.37-1.51 (m, 1H), 1.52-1.64 (m, 1H), 1.65-1.84 (m, 3H), 2.07-2.18 (m, 1H), 2.23 (s, 3H), 2.60-2.68 (m, 2H), 2.93 (dd, 1H), 3.13 (dd, 1H), 3.45-3.49 (m, 1H), 3.95-4.08 (m, 1H), 4.67-4.81 (m, 2H), 7.18-7.30 (m, 3H), 7.36 (d, 2H), 7.58 (d, 2H), 7.66-7.72 (m, 1H), 7.74-7.78 (m, 1H), 7.89 (d, 2H), 8.00-8.07 (m, 1H), 8.10-8.17 (m, 1H), 10.06-10.12 (m, 1H).

LC-MS (Method 4): Rₜ=0.75 min; MS (ESIpos): m/z=639.6 [M+H—HCl]⁺

### Example 140

### 4′-{(2S)-2-({[trans-4-(Aminomethyl)cyclohexyl]carbonyl}amino)-3-[(1-methyl-2-oxo-2,3-dihydro-1H-benzimidazol-5-yl)amino]-3-oxopropyl}-N-isopropyl-2-methylbiphenyl-4-carboxamide hydrochloride

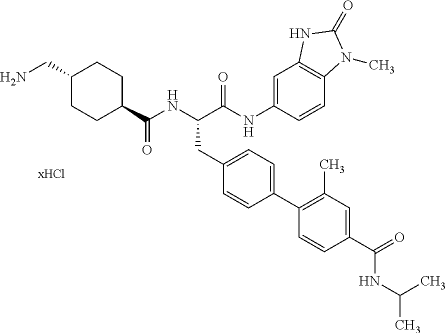 [SMILES: Cc1cc(C(=O)NC(C)C)ccc1-c1ccc(C[C@H](NC(=O)[C@H]2CC[C@H](CN)CC2)C(=O)Nc2ccc3c(c2)[nH]c(=O)n3C)cc1]

A solution of tert-butyl {[trans-4-({(2S)-3-[4′-(isopropylcarbamoyl)-2′-methylbiphenyl-4-yl]-1-[(1-methyl-2-oxo-2,3-dihydro-1H-benzimidazol-5-yl)amino]-1-oxopropan-2-yl}carbamoyl)-cyclohexyl]methyl}carbamate (51.1 mg, 0.07 mmol) in dioxane (3 ml) was admixed with 4M hydrogen chloride in 1,4-dioxane (0.26 ml, 1.06 mmol) and stirred at RT for 3 days. The solvent was removed on a rotary evaporator and the residue was dissolved in DMSO/acetonitrile. The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 28.6 mg (47% of theory) of the title compound.

¹H NMR (400 MHz, DMSO-d₆): δ=ppm 0.92 (d, 2H), 1.16 (s, 3H), 1.18 (s, 3H), 1.24 (s, 2H), 1.39-1.63 (m, 2H), 1.73 (d, 3H), 2.14 (br. s., 1H), 2.23 (s, 3H), 2.63 (t, 2H), 2.88-2.99 (m, 1H), 3.09 (d, 1H), 3.25 (s, 3H), 4.11 (m, 1H), 4.65-4.75 (m, 1H), 7.00 (d, 1H), 7.12 (dd, 1H), 7.21 (d, 1H), 7.25 (d, 2H), 7.37 (d, 2H), 7.50 (d, 1H), 7.71 (d, 1H), 7.76 (s, 1H), 7.80 (br. s., 3H), 8.18 (m, 2H), 10.06 (s, 1H), 10.80 (s, 1H).

LC-MS (Method 1): Rₜ=0.68 min; MS (ESIneg): m/z=623 [M−H—HCl]⁻.

### Example 141

### 4′-[(2S)-2-({[trans-4-(Aminomethyl)cyclohexyl]carbonyl}amino)-3-(1H-benzotriazol-5-ylamino)-3-oxopropyl]-N-cyclopropyl-2-methylbiphenyl-4-carboxamide hydrochloride

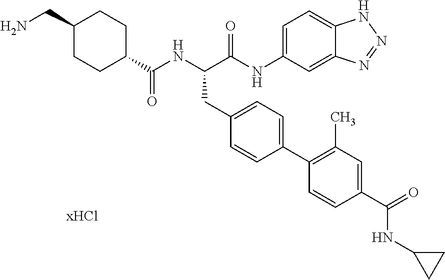 [SMILES: Cc1cc(C(=O)NC2CC2)ccc1-c1ccc(C[C@H](NC(=O)[C@H]2CC[C@H](CN)CC2)C(=O)Nc2ccc3[nH]nnc3c2)cc1]

A solution of tert-butyl {[trans-4-({(2S)-1-(1H-benzotriazol-5-ylamino)-3-[4′-(cyclopropyl-carbamoyl)-2′-methylbiphenyl-4-yl]-1-oxopropan-2-yl}carbamoyl)cyclohexyl]methyl}carbamate (43.4 mg, 0.063 mmol) in dioxane (2.5 ml) was admixed with 4M hydrogen chloride in 1,4-dioxane (0.24 ml, 0.94 mmol) and stirred at RT for 7 days. The solvent was removed on a rotary evaporator and the residue was dissolved in DMSO/acetonitrile. The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 28.6 mg (68% of theory) of the title compound.

¹H NMR (400 MHz, DMSO-d₆): δ=ppm 0.54-0.62 (m, 2H), 0.66-0.74 (m, 2H), 0.83-1.01 (m, 2H), 1.11-1.35 (m, 2H), 1.42-1.52 (m, 1H), 1.53-1.64 (m, 1H), 1.68-1.83 (m, 3H), 2.11-2.19 (m, 1H), 2.20 (s, 3H), 2.58-2.70 (m, 2H), 2.81-2.91 (m, 1H), 2.92-3.02 (m, 1H), 3.08-3.17 (m, 1H), 3.96 (s, 1H), 4.76-4.79 (m, 1H), 7.20 (d, 1H), 7.25 (d, 2H), 7.39 (d, 2H), 7.42-7.48 (m, 1H), 7.63-7.69 (m, 1H), 7.73 (s, 1H), 7.81 (br. s., 3H), 7.90 (d, 1H), 8.26-8.31 (m, 1H), 8.35 (s, 1H), 8.43 (d, 1H), 10.51 (s, 1H).

LC-MS (Method 1): Rₜ=0.65 min; MS (ESIneg): m/z=592 [M−H—HCl]⁻.

### Example 142

### 4′-[(2S)-2-({[trans-4-(Aminomethyl)cyclohexyl]carbonyl}amino)-3-(1H-benzimidazol-6-ylamino)-3-oxopropyl]-N-isopropyl-2-methylbiphenyl-4-carboxamide hydrochloride

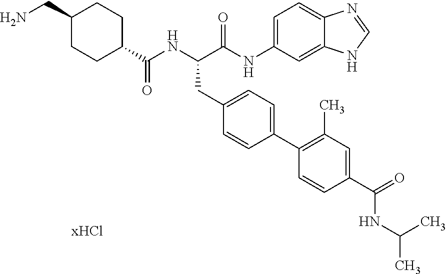 [SMILES: Cc1cc(C(=O)NC(C)C)ccc1-c1ccc(C[C@H](NC(=O)[C@H]2CC[C@H](CN)CC2)C(=O)Nc2ccc3nc[nH]c3c2)cc1]

A solution of tert-butyl {[trans-4-({(2S)-1-(1H-benzimidazol-6-ylamino)-3-[4′-(isopropyl-carbamoyl)-2′-methylbiphenyl-4-yl]-1-oxopropan-2-yl}carbamoyl)cyclohexyl]methyl}carbamate (66 mg, 0.095 mmol) in dioxane (3 ml) was admixed with 4M hydrogen chloride in 1,4-dioxane (0.36 ml, 1.425 mmol) and stirred at RT overnight. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 53.1 mg (89% of theory) of the title compound.

¹H NMR (400 MHz, DMSO-d₆): δ=ppm 0.80-1.03 (m, 2H), 1.17 (m, 7H), 1.28 (m, 1H), 1.42-1.53 (m, 1H), 1.58 (m, 1H), 1.76 (m, 3H), 2.10-2.19 (m, 1H), 2.22 (s, 3H), 2.63 (m, 2H), 2.95-3.03 (m, 1H), 3.16 (dd, 1H), 4.09 (m, 1H), 4.69-4.84 (m, 1H), 7.21 (d, 1H), 7.25 (d, 2H), 7.41 (d, 2H), 7.66 (dd, 1H), 7.71 (d, 1H), 7.76 (s, 1H), 7.80 (d, 1H), 7.88 (br. s., 3H), 8.20 (d, 1H), 8.30 (d, 1H), 8.39 (m, 1H), 9.49 (s, 1H), 10.69 (s, 1H).

LC-MS (Method 1): Rₜ=0.59 min; MS (ESIneg): m/z=593 [M−H—HCl]⁻.

### Example 143

### 4′-[(2S)-2-({[trans-4-(Aminomethyl)cyclohexyl]carbonyl}amino)-3-(1H-indazol-6-ylamino)-3-oxopropyl]-2-methyl-N-(2-oxopiperidin-3-yl)biphenyl-4-carboxamide hydrochloride

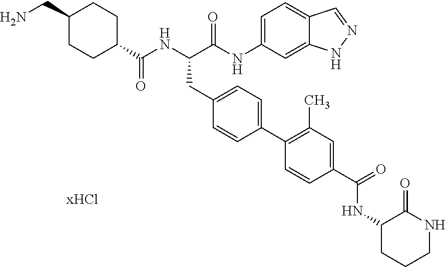

A solution of tert-butyl [(trans-4-{[(2S)-1-(1H-indazol-6-ylamino)-3-{2′-methyl-4′-[(2-oxopiperidin-3-yl)carbamoyl]biphenyl-4-yl}-1-oxopropan-2-yl]carbamoyl}cyclohexyl)methyl]-carbamate (42.7 mg, 0.057 mmol) in dioxane (2 ml) was admixed with 4M hydrogen chloride in 1,4-dioxane (0.21 ml, 0.85 mmol) and stirred at RT for 2 days. The precipitated solid was filtered off and washed with acetonitrile. The solid was dried under high vacuum. This gave 40 mg (100% of theory) of the title compound.

¹H NMR (400 MHz, DMSO-d₆): δ=ppm 0.82-1.01 (m, 2H), 1.10-1.34 (m, 2H), 1.54 (m, 2H), 1.68-1.91 (m, 6H), 2.00 (m, 1H), 2.10-2.20 (m, 1H), 2.23 (s, 3H), 2.63 (m, 2H), 2.97 (dd, 1H), 3.09-3.23 (m, 3H), 4.35-4.42 (m, 1H), 4.77 (m, 1H), 7.15 (d, 1H), 7.24 (d, 1H), 7.27 (d, 2H), 7.41 (d, 2H), 7.62-7.69 (m, 2H), 7.72 (d, 1H), 7.79 (s, 1H), 7.84 (br. s., 3H), 7.98 (m, 1H), 8.14 (s, 1H), 8.27 (d, 1H), 8.59 (d, 1H), 10.37 (s, 1H), 12.97 (br. s, 1H).

LC-MS (Method 1): Rₜ=0.67 min; MS (ESIneg): m/z=648 [M−H—HCl]⁻.

### Example 144

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-[4-(dimethylamino)-2,2-dimethylcyclohexyl]-2-methylbiphenyl-4-carboxamide hydrochloride

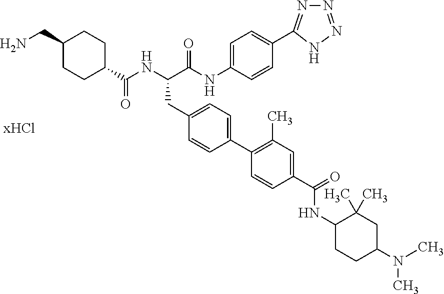 [SMILES: Cc1cc(C(=O)NC2CCC(N(C)C)CC2(C)C)ccc1-c1ccc(C[C@H](NC(=O)[C@H]2CC[C@H](CN)CC2)C(=O)Nc2ccc(-c3nnn[nH]3)cc2)cc1]

A solution of tert-butyl [(trans-4-{[(2S)-3-(4′-{[4-(dimethylamino)-2,2-dimethylcyclohexyl]-carbamoyl}-2′-methylbiphenyl-4-yl)-1-oxo-1-{[4-(1H-tetrazol-5-yl)phenyl]amino}propan-2-yl]carbamoyl}cyclohexyl)methyl]carbamate (38.5 mg, 0.046 mmol) in dioxane (1.5 ml) was admixed with 4M hydrogen chloride in 1,4-dioxane (0.17 ml, 0.69 mmol) and stirred at RT overnight. The precipitated solid was filtered off and washed with dioxane. The solid was dried under high vacuum. This gave 32.1 mg (85% of theory) of the title compound.

¹H NMR (400 MHz, DMSO-d₆): δ=ppm 0.86-1.00 (m, 6H), 1.06-1.97 (m, 15H), 2.11-2.20 (m, 1H), 2.21-2.29 (m, 3H), 2.59-2.76 (m, 7H), 2.90-3.03 (m, 1H), 3.06-3.21 (m, 1H), 3.85-4.02 (m, 1H), 4.68-4.85 (m, 1H), 7.13-7.30 (m, 3H), 7.40 (d, 2H), 7.57-7.91 (m, 8H), 8.02 (d, 3H), 8.29 (br. s., 1H), 10.01-10.30 (m, 1H), 10.42-10.65 (m, 1H).

LC-MS (Method 1): Rₜ=0.61 min; MS (ESIneg): m/z=732 [M−H—HCl]⁻.

### Example 145

### 4′-[(2S)-2-({[trans-4-(Aminomethyl)cyclohexyl]carbonyl}amino)-3-(1H-indazol-6-ylamino)-3-oxopropyl]-2-methyl-N-[(3R)-2-oxopyrrolidin-3-yl]biphenyl-4-carboxamide hydrochloride

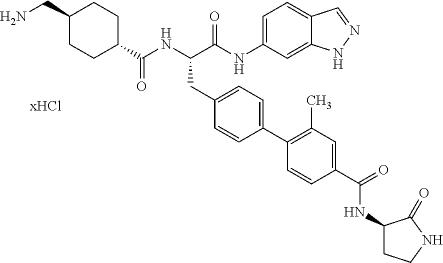 [SMILES: Cc1cc(C(=O)N[C@@H]2CCNC2=O)ccc1-c1ccc(C[C@H](NC(=O)[C@H]2CC[C@H](CN)CC2)C(=O)Nc2ccc3cn[nH]c3c2)cc1]

A solution of tert-butyl [(trans-4-{[(2S)-3-(4′-{[4-(dimethylamino)-2,2-dimethylcyclohexyl]-carbamoyl}-2′-methylbiphenyl-4-yl)-1-oxo-1-{[4-(1H-tetrazol-5-yl)phenyl]amino}propan-2-yl]carbamoyl}cyclohexyl)methyl]carbamate (90 mg, 0.046 mmol) in dioxane (2 ml) was admixed with 4M hydrogen chloride in 1,4-dioxane (0.15 ml, 0.61 mmol) and stirred at RT overnight. The precipitated solid was filtered off and washed with diethyl ether. The solid was dried under high vacuum. This gave 46.6 mg (53% of theory) of the title compound.

¹H NMR (400 MHz, DMSO-d₆): δ=ppm 0.82-1.02 (m, 2H), 1.07-1.37 (m, 3H), 1.41-1.51 (m, 1H), 1.54-1.62 (m, 1H), 1.66-1.84 (m, 3H), 1.93-2.07 (m, 1H), 2.12-2.20 (m, 1H), 2.23 (s, 3H), 2.30-2.41 (m, 1H), 2.59-2.69 (m, 2H), 2.90-3.02 (m, 1H), 3.10-3.16 (m, 1H), 3.20-3.29 (m, 2H), 4.50-4.63 (m, 1H), 4.71-4.84 (m, 1H), 7.13 (d, 1H), 7.25 (d, 1H), 7.28 (d, 2H), 7.40 (d, 2H), 7.67 (d, 1H), 7.70-7.83 (m, 5H), 7.84-7.89 (m, 1H), 7.98 (s, 1H), 8.13 (s, 1H), 8.25 (d, 1H), 8.66 (d, 1H), 10.23-10.43 (m, 1H).

LC-MS (Method 1): Rₜ=0.65 min; MS (ESIneg): m/z=634 [M−H—HCl]⁻.

### Example 146

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-{4-[benzyl(methyl)amino]cyclohexyl}-2-methylbiphenyl-4-carboxamide hydrochloride

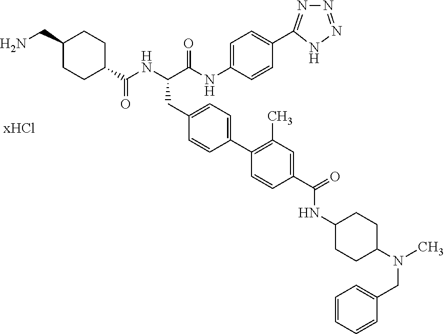 [SMILES: Cc1cc(C(=O)NC2CCC(N(C)Cc3ccccc3)CC2)ccc1-c1ccc(C[C@H](NC(=O)[C@H]2CC[C@H](CN)CC2)C(=O)Nc2ccc(-c3nnn[nH]3)cc2)cc1]

A solution of tert-butyl [(trans-4-{[(2S)-3-[4′-({4-[benzyl(methyl)amino]cyclohexyl}-carbamoyl)-2′-methylbiphenyl-4-yl]-1-oxo-1-{[4-(1H-tetrazol-5-yl)phenyl]amino}propan-2-yl]carbamoyl}cyclohexyl)methyl]carbamate (40 mg, 0.045 mmol) in dioxane (1 ml) was admixed with 4M hydrogen chloride in 1,4-dioxane (0.17 ml, 0.68 mmol) and stirred at RT overnight. The precipitated solid was filtered off and washed with dioxane. The solid was dried under high vacuum. This gave 35.7 mg (85% of theory) of the title compound.

¹H NMR (400 MHz, DMSO-d₆): δ=ppm 0.83-1.03 (m, 2H), 1.12-1.52 (m, 4H), 1.53-1.64 (m, 2H), 1.76 (m, 4H), 1.90-2.06 (m, 4H), 2.11-2.29 (m, 5H), 2.62 (br. s., 5H), 2.87-3.03 (m, 1H), 3.07-3.21 (m, 2H), 3.78-4.27 (m, 2H), 4.40-4.53 (m, 1H), 4.69-4.81 (m, 1H), 7.17-7.31 (m, 3H), 7.40 (d, 2H), 7.48 (m, 3H), 7.64 (m, 2H), 7.67-7.90 (m, 7H), 8.02 (d, 2H), 8.23-8.35 (m, 2H), 10.03-10.31 (m, 1H), 10.56 (br. s, 1H).

LC-MS (Method 1): Rₜ=0.60 min; MS (ESIneg): m/z=780 [M−H—HCl]⁻.

### Example 147

### 4′-[(2S)-2-({[trans-4-(Aminomethyl)cyclohexyl]carbonyl}amino)-3-oxo-3-{[4-(1H-tetrazol-5-yl)phenyl]amino}propyl]-2-methyl-N-[4-(methylamino)cyclohexyl]biphenyl-4-carboxamide hydrochloride

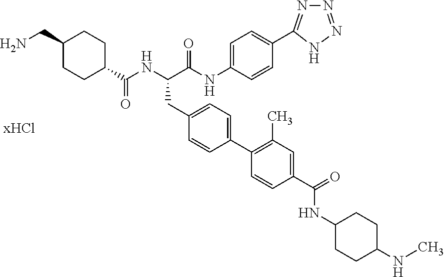 [SMILES: CNC1CCC(NC(=O)c2ccc(-c3ccc(C[C@H](NC(=O)[C@H]4CC[C@H](CN)CC4)C(=O)Nc4ccc(-c5nnn[nH]5)cc4)cc3)c(C)c2)CC1]

A solution of tert-butyl [(trans-4-{[(2S)-3-{2′-methyl-4′-{[4-(methylamino)cyclohexyl]-carbamoyl}biphenyl-4-yl)-1-oxo-1-{[4-(1H-tetrazol-5-yl)phenyl]amino}propan-2-yl]carbamoyl}-cyclohexyl)methyl]carbamate (55.9 mg, 0.071 mmol) in dioxane (2 ml) was admixed with 4M hydrogen chloride in 1,4-dioxane (0.27 ml, 1.06 mmol) and stirred at RT for 4 h. The precipitated solid was filtered off and washed with dioxane. The solid was dried under high vacuum. This gave 51.9 mg (84% of theory) of the title compound.

¹H NMR (400 MHz, DMSO-d₆): δ=ppm 0.84-1.01 (m, 2H), 1.12-1.31 (m, 3H), 1.35-1.53 (m, 4H), 1.55-1.67 (m, 2H), 1.69-1.86 (m, 5H), 1.89-2.00 (m, 2H), 2.06-2.20 (m, 2H), 2.22 (m, 3H), 2.60-2.70 (m, 2H), 2.87-3.22 (m, 4H), 3.68-3.99 (m, 1H), 4.70-4.82 (m, 1H), 7.11-7.32 (m, 3H), 7.41 (d, 2H), 7.59-7.88 (m, 7H), 7.95-8.09 (m, 2H), 8.17-8.37 (m, 2H), 8.51-8.80 (m, 2H), 10.52 (br. s, 1H), 16.81 (br. s, 1H).

LC-MS (Method 1): Rₜ=0.52 min; MS (ESIneg): m/z=690 [M−H—HCl]⁻.

### Example 148

### 4-[5-(4-{[(2S)-2-({[trans-4-(Aminomethyl)cyclohexyl]carbonyl}amino)-3-(4′-{[4-(dimethylamino)cyclohexyl]carbamoyl}-2′-methylbiphenyl-4-yl)propanoyl]amino}phenyl)-4H-1,2,4-triazol-3-yl]-2,2,3,3,4,4-hexafluorobutanoic acid hydrochloride

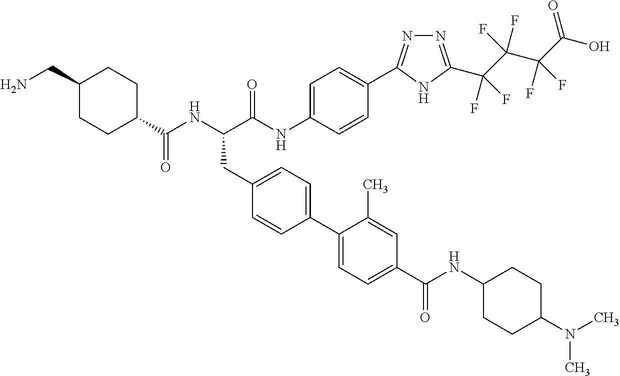 [SMILES: Cc1cc(C(=O)NC2CCC(N(C)C)CC2)ccc1-c1ccc(C[C@H](NC(=O)[C@H]2CC[C@H](CN)CC2)C(=O)Nc2ccc(-c3nnc(C(F)(F)C(F)(F)C(F)(F)C(=O)O)[nH]3)cc2)cc1]

A solution of 4-[5-(4-{[(2S)-2-{[(trans-4-{[(tert-butoxycarbonyl)amino]methyl}cyclohexyl)-carbonyl]amino}-3-(4′-{[4-(dimethylamino)cyclohexyl]carbamoyl}-2′-methylbiphenyl-4-yl)propanoyl]amino}phenyl)-4H-1,2,4-triazol-3-yl]-2,2,3,3,4,4-hexafluorobutanoic acid (11.2 mg, 0.011 mmol) in dioxane (1 ml) was admixed with 4M hydrogen chloride in 1,4-dioxane (0.04 ml, 0.17 mmol) and stirred at RT overnight. The precipitated solid was filtered off and washed with dioxane. The solid was dried under high vacuum. This gave 8.4 mg (74% of theory) of the title compound.

¹H NMR (400 MHz, DMSO-d₆): δ=ppm 0.84-1.03 (m, 2H), 1.13-1.28 (m, 1H), 1.38-1.51 (m, 3H), 1.53-1.65 (m, 3H), 1.68-1.84 (m, 3H), 1.92-2.00 (m, 2H), 2.02-2.09 (m, 2H), 2.10-2.19 (m, 1H), 2.22 (s, 3H), 2.60-2.68 (m, 2H), 2.72 (m, 7H), 2.90-3.02 (m, 1H), 3.07-3.22 (m, 2H), 4.66-4.82 (m, 1H), 7.12-7.30 (m, 3H), 7.35-7.43 (m, 2H), 7.67-7.73 (m, 1H), 7.79 (m, 6H), 7.94-8.05 (m, 2H), 8.17-8.38 (m, 2H), 10.07 (br. s, 1H), 10.49 (br. s, 1H), 15.18 (br. s, 1H).

LC-MS (Method 1): Rₜ=0.62 min; MS (ESIneg): m/z=897 [M−H—HCl]⁻.

### Example 149

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-(3,3-dimethylpiperidin-4-yl)-2-methylbiphenyl-4-carboxamide hydrochloride

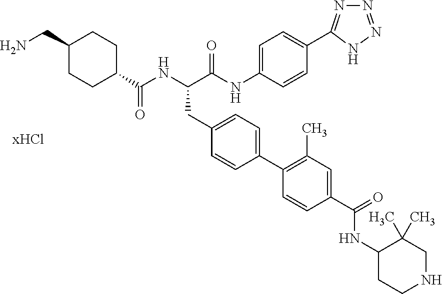 [SMILES: Cc1cc(C(=O)NC2CCNCC2(C)C)ccc1-c1ccc(C[C@H](NC(=O)[C@H]2CC[C@H](CN)CC2)C(=O)Nc2ccc(-c3nnn[nH]3)cc2)cc1]

A solution of tert-butyl 4-[({4′-[(2S)-2-{[(trans-4-{[(tert-butoxycarbonyl)amino]methyl}-cyclohexyl)carbonyl]amino}-3-oxo-3-{[4-(1H-tetrazol-5-yl)phenyl]amino}propyl]-2-methylbiphenyl-4-yl}carbonyl)amino]-3,3-dimethylpiperidine-1-carboxylate (75 mg, 0.084 mmol) in dioxane (3 ml) was admixed with 4M hydrogen chloride in 1,4-dioxane (0.31 ml, 1.26 mmol) and stirred at RT overnight. The precipitated solid was filtered off and washed with dioxane. The solid was dried under high vacuum. This gave 71 mg (quant.) of the title compound.

¹H NMR (400 MHz, DMSO-d₆): δ=ppm 0.88-0.98 (m, 5H), 1.06 (s, 3H), 1.12-1.35 (m, 2H), 1.43-1.53 (m, 1H), 1.54-1.63 (m, 1H), 1.74 (m, 4H), 1.87-2.04 (m, 1H), 2.11-2.20 (m, 1H), 2.23 (s, 3H), 2.59-2.69 (m, 2H), 2.84-3.18 (m, 6H), 4.10-4.22 (m, 1H), 4.70-4.83 (m, 1H), 7.25 (m, 3H), 7.40 (d, 2H), 7.69-7.91 (m, 7H), 8.02 (d, 2H), 8.16 (d, 1H), 8.30 (d, 1H), 8.52 (br. s, 1H), 9.05 (br. s, 1H), 10.51-10.62 (m, 1H).

LC-MS (Method 1): Rₜ=0.54 min; MS (ESIneg): m/z=690 [M−H—HCl]⁻.

### Example 150

### 4′-[(2S)-2-({[trans-4-(Aminomethyl)cyclohexyl]carbonyl}amino)-3-oxo-3-{[4-(1H-tetrazol-5-yl)phenyl]amino}propyl]-N-{4-[ethyl(methyl)amino]cyclohexyl}-2-methylbiphenyl-4-carboxamide hydrochloride

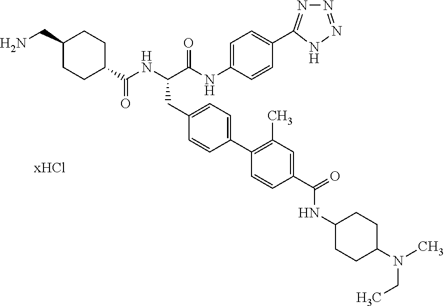 [SMILES: CCN(C)C1CCC(NC(=O)c2ccc(-c3ccc(C[C@H](NC(=O)[C@H]4CC[C@H](CN)CC4)C(=O)Nc4ccc(-c5nnn[nH]5)cc4)cc3)c(C)c2)CC1]

A solution of tert-butyl [(trans-4-{[(2S)-3-[4′-({4-[ethyl(methyl)amino]cyclohexyl)-carbamoyl}-2′-methylbiphenyl-4-yl]-1-oxo-1-{[4-(1H-tetrazol-5-yl)phenyl]amino}propan-2-yl]carbamoyl}cyclohexyl)methyl]carbamate (52 mg, 0.064 mmol) in dioxane (2 ml) was admixed with 4M hydrogen chloride in 1,4-dioxane (0.24 ml, 0.95 mmol) and stirred at RT overnight. The precipitated solid was filtered off and washed with dioxane. The solid was dried under high vacuum. This gave 45 mg (86% of theory) of the title compound.

¹H NMR (400 MHz, DMSO-d₆): δ=ppm 0.82-1.01 (m, 2H), 1.22 (m, 5H), 1.39-1.53 (m, 2H), 1.54-1.69 (m, 3H), 1.69-1.90 (m, 5H), 1.93-2.20 (m, 4H), 2.23 (m, 3H), 2.58-2.71 (m, 5H), 2.91-3.09 (m, 2H), 3.11-3.27 (m, 3H), 3.72-4.17 (m, 1H), 4.68-4.83 (m, 1H), 7.24 (m, 3H), 7.40 (d, 2H), 7.66-7.76 (m, 2H), 7.83 (m, 5H), 8.06 (m, 2H), 8.24-8.38 (m, 2H), 10.02 (br. s, 1H), 10.56 (br. s., 1H).

LC-MS (Method 1): Rₜ=0.54 min; MS (ESIneg): m/z=718 [M−H—HCl]⁻.

### Example 151

### Methyl 5-[({4′-[(2S)-2-({[trans-4-(aminomethyl)cyclohexyl]carbonyl}amino)-3-oxo-3-{[4-(1H-tetrazol-5-yl)phenyl]amino}propyl]-2-methylbiphenyl-4-yl}carbonyl)amino]-6-oxopiperidine-2-carboxylate hydrochloride

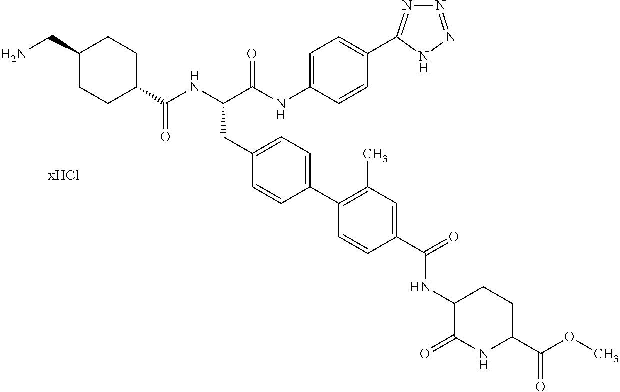 [SMILES: COC(=O)C1CCC(NC(=O)c2ccc(-c3ccc(C[C@H](NC(=O)[C@H]4CC[C@H](CN)CC4)C(=O)Nc4ccc(-c5nnn[nH]5)cc4)cc3)c(C)c2)C(=O)N1]

A solution of methyl 5-[({4′-[(2S)-2-{[(trans-4-{[(tert-butoxycarbonyl)amino]methyl}-cyclohexyl)carbonyl]amino}-3-oxo-3-{[4-(1H-tetrazol-5-yl)phenyl]amino}propyl]-2-methylbiphenyl-4-yl}carbonyl)amino]-6-oxopiperidine-2-carboxylate (35 mg, 0.042 mmol) in dioxane (1.5 ml) was admixed with 4M hydrogen chloride in 1,4-dioxane (0.16 ml, 0.628 mmol) and stirred at RT for 3 days. The precipitated solid was filtered off and washed with dioxane. The solid was dried under high vacuum. This gave 30 mg (84% of theory) of the title compound.

¹H NMR (400 MHz, DMSO-d₆): δ=ppm 0.84-1.02 (m, 2H), 1.10-1.35 (m, 2H), 1.41-1.51 (m, 1H), 1.58 (d, 1H), 1.68-1.83 (m, 4H), 1.85-2.02 (m, 2H), 2.10-2.20 (m, 2H), 2.23 (s, 3H), 2.60-2.69 (m, 2H), 2.89-3.02 (m, 1H), 3.09-3.19 (m, 1H), 4.07-4.20 (m, 1H), 4.34-4.60 (m, 1H), 4.70-4.83 (m, 1H), 7.29 (s, 3H), 7.36-7.44 (m, 2H), 7.68-7.87 (m, 8H), 8.01 (d, 2H), 8.26 (d, 1H), 8.58-8.70 (m, 1H), 10.51 (br. s, 1H).

LC-MS (Method 1): Rₜ=0.67 min; MS (ESIneg): m/z=734 [M−H—HCl]⁻.

### Example 152

### 3-{(5-[4-({(2S)-2-({[trans-4-(Aminomethyl)cyclohexyl]carbonyl}amino)-3-[2′-methyl-4′-(piperidin-4-ylcarbamoyl)biphenyl-4-yl]propanoyl}amino)phenyl]-4H-1,2,4-triazol-3-yl}propanoic acid hydrochloride

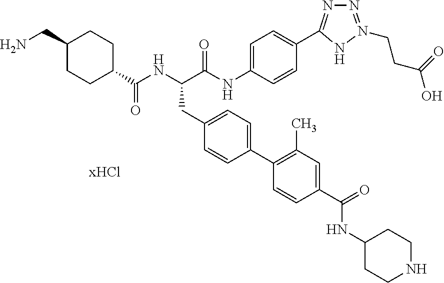

A solution of tert-butyl 4-[({4′-[(2S)-2-{[(trans-4-{[(tert-butoxycarbonyl)amino]methyl}-cyclohexyl)carbonyl]amino}-3-({4-[5-(3-tert-butoxy-3-oxopropyl)-4H-1,2,4-triazol-3-yl]phenyl}-amino)-3-oxopropyl]-2-methylbiphenyl-4-yl}carbonyl)amino]piperidine-1-carboxylate (69 mg, 0.07 mmol) in dioxane (2 ml) was admixed with 4M hydrogen chloride in 1,4-dioxane (0.26 ml, 1.04 mmol) and stirred at RT overnight. The precipitated solid was filtered off and washed with dioxane. The solid was dried under high vacuum. This gave 56 mg (100% of theory) of the title compound.

¹H NMR (400 MHz, DMSO-d₆): δ=ppm 0.83-1.01 (m, 2H), 1.11-1.34 (m, 2H), 1.41-1.52 (m, 1H), 1.54-1.63 (m, 1H), 1.78 (d, 5H), 1.91-2.01 (m, 2H), 2.08 (s, 1H), 2.11-2.19 (m, 1H), 2.24 (s, 3H), 2.58-2.67 (m, 2H), 2.72-2.81 (m, 2H), 2.90-3.07 (m, 5H), 3.09-3.18 (m, 1H), 3.26-3.36 (m, 2H), 4.70-4.79 (m, 1H), 7.20-7.29 (m, 3H), 7.40 (d, 2H), 7.73 (d, 3H), 7.79 (s, 1H), 7.85 (br. s., 3H), 7.95 (d, 2H), 8.27 (d, 1H), 8.49 (d, 1H), 8.71-8.98 (m, 2H), 10.43 (br. s, 1H).

LC-MS (Method 1): Rₜ=0.53 min; MS (ESIneg): m/z=733 [M−H—HCl]⁻.

### Example 153

### 3-[5-(4-{[(2S)-2-({[trans-4-(Aminomethyl)cyclohexyl]carbonyl}amino)-3-{2′-methyl-4′-[(3S)-pyrrolidin-3-ylcarbamoyl]biphenyl-4-yl}propanoyl]amino}phenyl)-4H-1,2,4-triazol-3-yl]-2,2,3,3-tetrafluoropropanoic acid hydrochloride

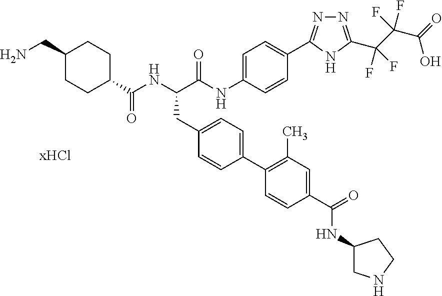 [SMILES: Cc1cc(C(=O)N[C@H]2CCNC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

A solution of 3-[5-(4-{[(2S)-2-{[(trans-4-{[(tert-butoxycarbonyl)amino]methyl}cyclohexyl)-carbonyl]amino}-3-(4′-{[(3S)-1-(tert-butoxycarbonyl)pyrrolidin-3-yl]carbamoyl-2′-methyl-biphenyl-4-yl)propanoyl]amino}phenyl)-4H-1,2,4-triazol-3-yl]-2,2,3,3-tetrafluoropropanoic acid (59.9 mg, 0.060 mmol) in dioxane (2 ml) was admixed with 4M hydrogen chloride in 1,4-dioxane (0.23 ml, 0.91 mmol) and stirred at RT overnight. The precipitated solid was filtered off and washed with dioxane. The solid was dried under high vacuum. This gave 52 mg (98% of theory) of the title compound.

¹H NMR (400 MHz, DMSO-d₆): δ=ppm 0.79-1.03 (m, 2H), 1.11-1.36 (m, 2H), 1.41-1.54 (m, 1H), 1.55-1.64 (m, 1H), 1.76 (br. s., 3H), 1.94-2.06 (m, 1H), 2.17 (d, 1H), 2.22 (s, 3H), 2.59-2.69 (m, 2H), 2.91-3.01 (m, 1H), 3.09-3.46 (m, 6H), 4.56 (d, 1H), 4.74 (d, 1H), 7.16-7.31 (m, 3H), 7.39 (d, 2H), 7.70-7.86 (m, 7H), 7.97 (d, 2H), 8.28 (d, 1H), 8.73 (d, 1H), 9.03 (br. s, 1H), 9.29 (br. s, 1H), 10.47 (br. s., 1H), 15.12 (br. s, 1H).

LC-MS (Method 1): Rₜ=0.55 min; MS (ESIneg): m/z=791 [M−H—HCl]⁻.

### Example 154

### 3-(5-[4-({(2S)-2-({[trans-4-(Aminomethyl)cyclohexyl]carbonyl}amino)-3-[4′-(3-azabicyclo[3.1.0]hex-6-ylcarbamoyl)-2′-methylbiphenyl-4-yl]propanoyl}amino)phenyl]-4H-1,2,4-triazol-3-yl}-2,2,3,3-tetrafluoropropanoic acid hydrochloride

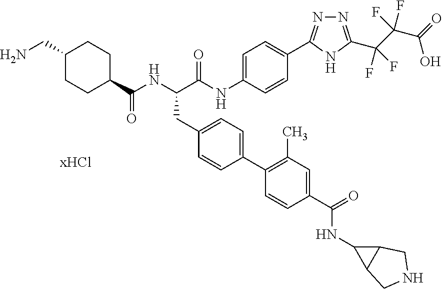 [SMILES: Cc1cc(C(=O)NC2C3CNCC32)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

A solution of 3-[5-(4-{[(2S)-2-{[(trans-4-{[(tert-butoxycarbonyl)amino]methyl}cyclohexyl)-carbonyl]amino}-3-(4′-{[3-(tert-butoxycarbonyl)-3-azabicyclo[3.1.0]hex-6-yl]carbamoyl}-2′-methylbiphenyl-4-yl)propanoyl]amino}phenyl)-4H-1,2,4-triazol-3-yl]-2,2,3,3-tetrafluoropropanoic acid (59.1 mg, 0.059 mmol) in dioxane (2 ml) was admixed with 4M hydrogen chloride in 1,4-dioxane (0.22 ml, 0.88 mmol) and stirred at RT overnight. The precipitated solid was filtered off and washed with dioxane. The solid was dried under high vacuum. This gave 50 mg (97% of theory) of the title compound.

¹H NMR (400 MHz, DMSO-d₆): δ=ppm 0.92 (br. s., 2H), 1.12-1.35 (m, 2H), 1.41-1.52 (m, 1H), 1.54-1.64 (m, 1H), 1.68-1.83 (m, 3H), 2.01 (br. s., 2H), 2.11-2.20 (m, 1H), 2.22 (s, 3H), 2.59-2.69 (m, 2H), 2.91-3.00 (m, 1H), 3.03 (br. s., 1H), 3.13 (m, 1H), 3.30-3.46 (m, 4H), 4.68-4.79 (m, 1H), 7.18-7.30 (m, 3H), 7.39 (d, 2H), 7.68 (d, 1H), 7.71-7.85 (m, 6H), 7.97 (d, 2H), 8.27 (d, 1H), 8.58 (d, 1H), 8.83 (br. s, 1H), 9.38 (br. s, 1H), 10.48 (br. s., 1H), 15.10 (br. s, 1H).

LC-MS (Method 1): Rₜ=0.55 min; MS (ESIneg): m/z=803 [M−H—HCl]⁻.

### Example 155

### 3-[5-(4-{[(2S)-2-({[trans-4-(Aminomethyl)cyclohexyl]carbonyl}amino)-3-{2′-methyl-4′-[(2-oxopiperidin-3-yl)carbamoyl]biphenyl-4-yl}propanoyl]amino}phenyl)-4H-1,2,4-triazol-3-yl]-2,2,3,3-tetrafluoropropanoic acid hydrochloride

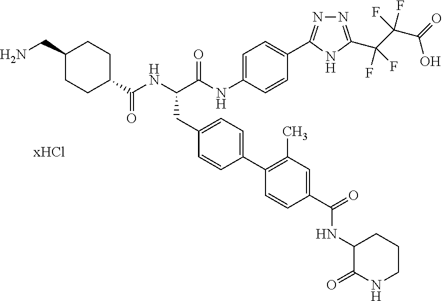 [SMILES: Cc1cc(C(=O)NC2CCCNC2=O)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

A solution of 3-[5-(4-{[(2S)-2-{[(trans-4-{[(tert-butoxycarbonyl)amino]methyl}cyclohexyl)-carbonyl]amino}-3-{2′-methyl-4′-[(2-oxopiperidin-3-yl)carbamoyl]biphenyl-4-yl}propanoyl]-amino}phenyl)-4H-1,2,4-triazol-3-yl]-2,2,3,3-tetrafluoropropanoic acid (33.7 mg, 0.037 mmol) in dioxane (1 ml) was admixed with 4M hydrogen chloride in 1,4-dioxane (0.14 ml, 0.55 mmol) and stirred at RT overnight. The precipitated solid was filtered off and washed with dioxane. The solid was dried under high vacuum. This gave 32 mg (93% of theory) of the title compound.

¹H NMR (400 MHz, DMSO-d₆): δ=ppm 0.84-1.01 (m, 2H), 1.10-1.34 (m, 2H), 1.38-1.51 (m, 1H), 1.58 (d, 1H), 1.78 (m, 6H), 1.95-2.05 (m, 1H), 2.10-2.20 (m, 1H), 2.24 (s, 3H), 2.64 (br. s., 2H), 2.90-3.00 (m, 1H), 3.08-3.22 (m, 3H), 4.33-4.43 (m, 1H), 4.69-4.81 (m, 1H), 7.18-7.32 (m, 3H), 7.40 (d, 2H), 7.55-7.86 (m, 8H), 7.97 (d, 2H), 8.24 (d, 1H), 8.59 (d, 1H), 10.48 (br. s, 1H), 15.13 (br. s, 1H).

LC-MS (Method 1): Rₜ=0.69 min; MS (ESIneg): m/z=819 [M−H—HCl]⁻.

### Example 156

### 4′-{(2S)-2-({[trans-4-(Aminomethyl)cyclohexyl]carbonyl}amino)-3-[(7-chloro-2-oxo-2,3-dihydro-1,3-benzoxazol-5-yl)amino]-3-oxopropyl}-N-isopropyl-2-methylbiphenyl-4-carboxamide hydrochloride

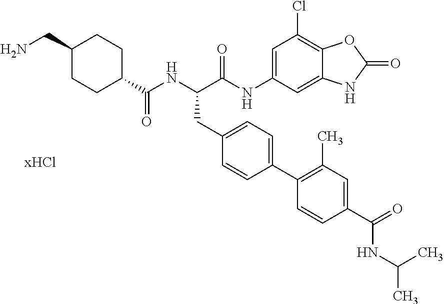 [SMILES: Cc1cc(C(=O)NC(C)C)ccc1-c1ccc(C[C@H](NC(=O)[C@H]2CC[C@H](CN)CC2)C(=O)Nc2cc(Cl)c3oc(=O)[nH]c3c2)cc1]

A solution of tert-butyl {[trans-4-({(2S)-1-[(7-chloro-2-oxo-2,3-dihydro-1,3-benzoxazol-5-yl)amino]-3-[4′-(isopropylcarbamoyl)-2′-methylbiphenyl-4-yl]-1-oxopropan-2-yl}carbamoyl)-cyclohexyl]methyl}carbamate (21.8 mg, 0.029 mmol) in dioxane (1.5 ml) was admixed with 4M hydrogen chloride in 1,4-dioxane (0.11 ml, 0.438 mmol) and stirred at RT for 3 days. Subsequently, additional 4M hydrogen chloride in 1,4-dioxane (0.11 ml, 0.438 mmol) was added and the mixture was stirred at RT for a further 10 days. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 7.3 mg (37% of theory) of the title compound.

¹H NMR (400 MHz, DMSO-d₆): δ=ppm 0.82-1.01 (m, 2H), 1.16 (s, 3H), 1.18 (s, 3H), 1.21-1.34 (m, 2H), 1.39-1.51 (m, 1H), 1.53-1.62 (m, 1H), 1.65-1.82 (m, 3H), 2.11-2.18 (m, 1H), 2.21 (s, 3H), 2.59-2.70 (m, 2H), 2.89-2.99 (m, 1H), 3.05-3.13 (m, 1H), 4.06-4.17 (m, 1H), 4.59-4.73 (m, 1H), 7.21 (d, 1H), 7.25 (d, 2H), 7.36 (d, 3H), 7.41-7.46 (m, 1H), 7.64-7.81 (m, 5H), 8.13-8.26 (m, 2H), 10.25-10.36 (m, 1H), 11.93 (br. s, 1H).

LC-MS (Method 1): Rₜ=0.79 min; MS (ESIneg): m/z=644 [M−H—HCl]⁻.

### Example 157

### 3-{5-[4-({(2S)-2-({[trans-4-(Aminomethyl)cyclohexyl]carbonyl}amino)-3-[4′-(isopropyl-carbamoyl)-2′-methylbiphenyl-4-yl]propanoyl}amino)phenyl]-4H-1,2,4-triazol-3-yl}propanoic acid hydrochloride

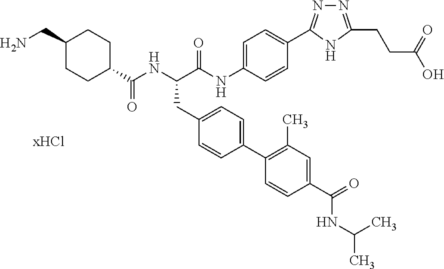 [SMILES: Cc1cc(C(=O)NC(C)C)ccc1-c1ccc(C[C@H](NC(=O)[C@H]2CC[C@H](CN)CC2)C(=O)Nc2ccc(-c3nnc(CCC(=O)O)[nH]3)cc2)cc1]

A solution of tert-butyl 3-{5-[4-({(2S)-2-{[(trans-4-{[(tert-butoxycarbonyl)amino]methyl}-cyclohexyl)carbonyl]amino}-3-[4′-(isopropylcarbamoyl)-2′-methylbiphenyl-4-yl]propanoyl}-amino)phenyl]-4H-1,2,4-triazol-3-yl}propanoate (58.1 mg, 0.068 mmol) in dioxane (2.5 ml) was admixed with 4M hydrogen chloride in 1,4-dioxane (0.26 ml, 1.025 mmol) and stirred at RT for 3 days. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 32 mg (61% of theory) of the title compound.

¹H NMR (400 MHz, DMSO-d₆): δ=ppm 0.82-1.00 (m, 2H), 1.16 (s, 3H), 1.18 (s, 3H), 1.21-1.33 (m, 1H), 1.42-1.51 (m, 1H), 1.52-1.62 (m, 1H), 1.76 (s, 4H), 2.10-2.19 (m, 1H), 2.23 (s, 3H), 2.58-2.69 (m, 2H), 2.74-2.84 (m, 2H), 2.89-3.06 (m, 3H), 3.09-3.18 (m, 1H), 4.03-4.18 (m, 1H), 4.69-4.80 (m, 1H), 7.22 (d, 1H), 7.26 (d, 2H), 7.40 (d, 2H), 7.71 (d, 1H), 7.76 (m, 3H), 7.85 (br. s, 3H), 7.93-8.01 (m, 2H), 8.19 (d, 1H), 8.22-8.28 (m, 1H), 10.44 (br. s, 1H).

LC-MS (Method 1): Rₜ=0.69 min; MS (ESIneg): m/z=692 [M−H—HCl]⁻.

### Example 158

### 4′-{(2S)-2-({[trans-4-(Aminomethyl)cyclohexyl]carbonyl}amino)-3-[(7-chloro-2-oxo-2,3-dihydro-1H-benzimidazol-5-yl)amino]-3-oxopropyl}-N-isopropyl-2-methylbiphenyl-4-carboxamide hydrochloride

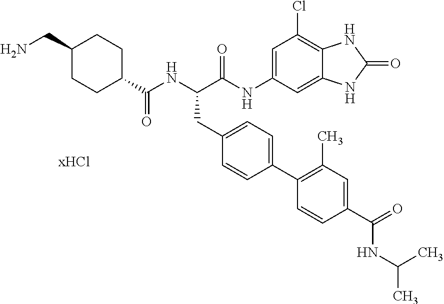 [SMILES: Cc1cc(C(=O)NC(C)C)ccc1-c1ccc(C[C@H](NC(=O)[C@H]2CC[C@H](CN)CC2)C(=O)Nc2cc(Cl)c3[nH]c(=O)[nH]c3c2)cc1]

A solution of tert-butyl {[trans-4-({(2S)-1-[(7-chloro-2-oxo-2,3-dihydro-1H-benzimidazol-5-yl)amino]-3-[4′-(isopropylcarbamoyl)-2′-methylbiphenyl-4-yl]-1-oxopropan-2-yl}carbamoyl)-cyclohexyl]methyl}carbamate (75 mg, 0.101 mmol) in dioxane (2 ml) was admixed with 4M hydrogen chloride in 1,4-dioxane (0.38 ml, 1.51 mmol) and stirred at RT overnight. The precipitated solid was filtered off and washed with dioxane/acetonitrile. The solid was dried under high vacuum. This gave 7.3 mg (37% of theory) of the title compound.

¹H NMR (400 MHz, DMSO-d₆): δ=ppm 0.82-1.00 (m, 2H), 1.16 (s, 3H), 1.18 (s, 3H), 1.20-1.34 (m, 2H), 1.40-1.50 (m, 1H), 1.54-1.62 (m, 1H), 1.68-1.82 (m, 3H), 2.09-2.19 (m, 1H), 2.22 (s, 3H), 2.59-2.70 (m, 2H), 2.87-2.98 (m, 1H), 3.03-3.14 (m, 1H), 4.04-4.16 (m, 1H), 4.61-4.72 (m, 1H), 7.21 (d, 1H), 7.23-7.27 (m, 3H), 7.29 (d, 1H), 7.36 (d, 2H), 7.64-7.82 (m, 5H), 8.18 (d, 2H), 10.11 (s, 1H), 10.87 (d, 1H), 11.05 (s, 1H).

LC-MS (Method 1): Rₜ=0.71 min; MS (ESIneg): m/z=643 [M−H—HCl]⁻.

### Example 159

### 4′-{(2S)-2-({[trans-4-(Aminomethyl)cyclohexyl]carbonyl}amino)-3-[(4-chloro-1H-indazol-6-yl)amino]-3-oxopropyl}-N-isopropyl-2-methylbiphenyl-4-carboxamide hydrochloride

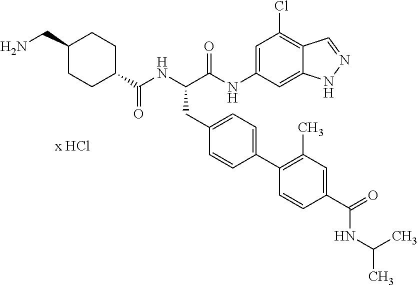 [SMILES: Cc1cc(C(=O)NC(C)C)ccc1-c1ccc(C[C@H](NC(=O)[C@H]2CC[C@H](CN)CC2)C(=O)Nc2cc(Cl)c3cn[nH]c3c2)cc1]

A solution of tert-butyl {[trans-4-({(2S)-1-[(4-chloro-1H-indazol-6-yl)amino]-3-[4′-(isopropyl-carbamoyl)-2′-methylbiphenyl-4-yl]-1-oxopropan-2-yl}carbamoyl)cyclohexyl]methyl}carbamate (21 mg, 0.023 mmol) in dioxane (1 ml) was admixed with 4M hydrogen chloride in 1,4-dioxane (0.09 ml, 0.34 mmol) and stirred at RT overnight. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 12 mg (79% of theory) of the title compound.

¹H NMR (400 MHz, DMSO-d₆): δ=ppm 0.85-1.00 (m, 2H), 1.16 (s, 3H), 1.18 (s, 3H), 1.20-1.35 (m, 2H), 1.42-1.50 (m, 1H), 1.56-1.64 (m, 1H), 1.69-1.81 (m, 3H), 2.11-2.17 (m, 1H), 2.20 (s, 3H), 2.60-2.70 (m, 2H), 2.97 (dd, 1H), 3.11 (dd, 1H), 4.04-4.17 (m, 1H), 4.66-4.78 (m, 1H), 7.20 (d, 1H), 7.25 (d, 2H), 7.29-7.32 (m, 1H), 7.37 (d, 2H), 7.63-7.73 (m, 4H), 7.73-7.78 (m, 1H), 8.02 (d, 2H), 8.18 (d, 1H), 8.24 (d, 1H), 10.36 (br. s, 1H).

LC-MS (Method 1): Rₜ=0.77 min; MS (ESIneg): m/z=627 [M−H—HCl]⁻.

### Example 160

### 6-({(2S)-2-({[trans-4-(Aminomethyl)cyclohexyl]carbonyl}amino)-3-[4′-(cyclobutylcarbamoyl)-2′-methylbiphenyl-4-yl]propanoyl}amino)-1H-indole-2-carboxylic acid hydrochloride

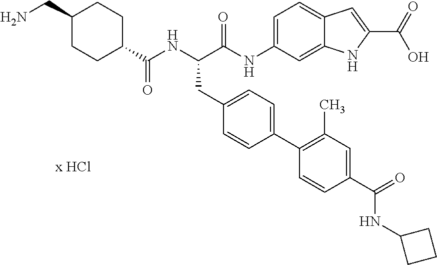 [SMILES: Cc1cc(C(=O)NC2CCC2)ccc1-c1ccc(C[C@H](NC(=O)[C@H]2CC[C@H](CN)CC2)C(=O)Nc2ccc3cc(C(=O)O)[nH]c3c2)cc1]

A solution of 6-({(2S)-2-{[(trans-4-{[(tert-butoxycarbonyl)amino]methyl}cyclohexyl)-carbonyl]amino}-3-[4′-(cyclobutylcarbamoyl)-2′-methylbiphenyl-4-yl]propanoyl}amino)-1H-indole-2-carboxylic acid (23 mg, 0.031 mmol) in dioxane (1.2 ml) was admixed with 4M hydrogen chloride in 1,4-dioxane (0.12 ml, 0.46 mmol) and stirred at RT overnight. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 16 mg (75% of theory) of the title compound.

¹H NMR (400 MHz, DMSO-d₆): δ=ppm 0.84-1.01 (m, 2H), 1.12-1.35 (m, 2H), 1.42-1.52 (m, 1H), 1.54-1.62 (m, 1H), 1.63-1.83 (m, 5H), 2.01-2.28 (m, 8H), 2.64 (br. s., 2H), 2.95 (dd, 1H), 3.13 (dd, 1H), 4.37-4.49 (m, 1H), 4.70-4.83 (m, 1H), 7.03 (d, 1H), 7.16 (dd, 1H), 7.22 (d, 1H), 7.26 (d, 2H), 7.39 (d, 2H), 7.55 (d, 1H), 7.67-7.81 (m, 5H), 7.98 (s, 1H), 8.17 (d, 1H), 8.58 (d, 1H), 10.17 (s, 1H), 11.66 (s, 1H).

LC-MS (Method 1): Rₜ=0.77 min; MS (ESIneg): m/z=648 [M−H—HCl]⁻.

### Example 161

### 6-({(2S)-2-({[trans-4-(Aminomethyl)cyclohexyl]carbonyl}amino)-3-[4′-(isopropylcarbamoyl)-2′-methylbiphenyl-4-yl]propanoyl}amino)-1H-indazole-4-carboxylic acid hydrochloride

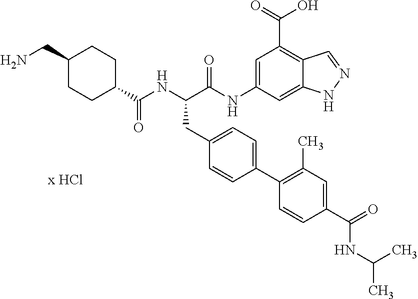 [SMILES: Cc1cc(C(=O)NC(C)C)ccc1-c1ccc(C[C@H](NC(=O)[C@H]2CC[C@H](CN)CC2)C(=O)Nc2cc(C(=O)O)c3cn[nH]c3c2)cc1]

A solution of 6-({(2S)-2-{[(trans-4-{[(tert-butoxycarbonyl)amino]methyl}cyclohexyl)-carbonyl]amino}-3-[4′-(isopropylcarbamoyl)-2′-methylbiphenyl-4-yl]propanoyl}amino)-1H-indazole-4-carboxylic acid (18 mg, 0.024 mmol) in dioxane (1 ml) was admixed with 4M hydrogen chloride in 1,4-dioxane (0.09 ml, 0.37 mmol) and stirred at RT overnight. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 3.3 mg (20% of theory) of the title compound.

¹H NMR (400 MHz, DMSO-d₆): δ=ppm 0.79-0.97 (m, 2H), 1.16 (s, 3H), 1.18 (s, 3H), 1.21-1.32 (m, 2H), 1.35-1.49 (m, 1H), 1.56-1.65 (m, 1H), 1.67-1.82 (m, 3H), 2.23 (br. M, 1H), 2.23 (s, 3H), 2.56-2.61 (m, 2H), 3.00-3.09 (m, 1H), 3.12-3.19 (m, 1H), 4.05-4.17 (m, 1H), 4.64-4.75 (m, 1H), 7.23 (d, 1H), 7.26 (d, 2H), 7.43 (d, 2H), 7.70 (d, 1H), 7.73-7.78 (m, 1H), 7.91 (br. s, 1H), 8.18 (d, 1H), 8.26-8.31 (m, 1H), 8.35-8.42 (m, 1H), 8.69 (br. s, 1H), 10.64 (br. s, 1H), 12.84 (br. s, 1H).

LC-MS (Method 1): Rₜ=0.68 min; MS (ESIneg): m/z=637 [M−H—HCl]⁻.

### Example 162

### 4′-{(2S)-2-({[trans-4-(Aminomethyl)cyclohexyl]carbonyl}amino)-3-oxo-3-[(3-oxo-2,3-dihydro-1H-indazol-6-yl)amino]propyl}-N-cyclobutyl-2-methylbiphenyl-4-carboxamide hydrochloride

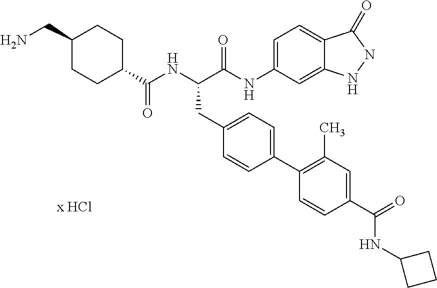 [SMILES: Cc1cc(C(=O)NC2CCC2)ccc1-c1ccc(C[C@H](NC(=O)[C@H]2CC[C@H](CN)CC2)C(=O)Nc2ccc3c(=O)[nH][nH]c3c2)cc1]

A solution of tert-butyl {[trans-4-({(2S)-3-[4′-(cyclobutylcarbamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}-carbamate (24 mg, 0.033 mmol) in dioxane (2 ml) was admixed with 4M hydrogen chloride in 1,4-dioxane (0.12 ml, 0.5 mmol) and stirred at RT overnight. The precipitated solid was filtered off and washed with dioxane/acetonitrile. The solid was dried under high vacuum. This gave 21 mg (89% of theory) of the title compound.

¹H NMR (400 MHz, DMSO-d₆): δ=ppm 0.82-1.00 (m, 2H), 1.10-1.34 (m, 2H), 1.41-1.52 (m, 1H), 1.57 (d, 1H), 1.64-1.82 (m, 5H), 2.01-2.20 (m, 4H), 2.23 (s, 3H), 2.59-2.68 (m, 2H), 2.95 (dd, 1H), 3.14 (dd, 1H), 4.37-4.49 (m, 1H), 4.70-4.80 (m, 1H), 7.03 (d, 1H), 7.22 (d, 1H), 7.26 (d, 2H), 7.39 (d, 2H), 7.55 (d, 1H), 7.71 (dd, 1H), 7.74-7.93 (m, 5H), 8.24 (d, 1H), 8.59 (d, 1H), 10.33 (br. s, 1H), 11.25 (br. s, 1H).

LC-MS (Method 1): Rₜ=0.66 min; MS (ESIneg): m/z=621 [M−H—HCl]⁻.

### Example 163

### trans-4-(Aminomethyl)-N-[(2S)-1-(1H-indazol-6-ylamino)-3-{2′-methyl-4′-[(6-oxohexahydropyrrolo[3,4-b]pyrrol-1(2H)-yl)carbonyl]biphenyl-4-yl}-1-oxopropan-2-yl]cyclohexanecarboxamide hydrochloride

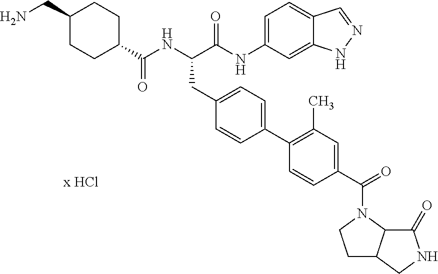 [SMILES: Cc1cc(C(=O)N2CCC3CNC(=O)C32)ccc1-c1ccc(C[C@H](NC(=O)[C@H]2CC[C@H](CN)CC2)C(=O)Nc2ccc3cn[nH]c3c2)cc1]

A solution of tert-butyl [(trans-4-{[(2S)-1-(1H-indazol-6-ylamino)-3-{2′-methyl-4′-[(6-oxohexahydropyrrolo[3,4-b]pyrrol-1 (2H)-yl)carbonyl]biphenyl-4-yl}-1-oxopropan-2-yl]-carbamoyl}cyclohexyl)methyl]carbamate (53.2 mg, 0.07 mmol) in dioxane (2 ml) was admixed with 4M hydrogen chloride in 1,4-dioxane (0.26 ml, 1.05 mmol) and stirred at RT overnight. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 40.9 mg (80% of theory) of the title compound.

¹H NMR (400 MHz, DMSO-d₆): δ=ppm 0.85-1.00 (m, 2H), 1.15 (m, 1H), 1.21-1.35 (m, 1H), 1.48 (br. s., 1H), 1.57 (d, 1H), 1.66-1.82 (m, 4H), 2.02-2.34 (m, 5H), 2.57-2.69 (m, 2H), 2.89-3.06 (m, 3H), 3.10-3.16 (m, 1H), 3.22-3.51 (m, 2H), 3.87 (br. s., 1H), 4.50 (m, 1H), 4.78 (m, 1H), 7.17 (d, 2H), 7.25 (d, 2H), 7.40 (d, 2H), 7.67 (d, 1H), 7.71-8.02 (m, 6H), 8.14 (s, 1H), 8.26 (d, 1H), 10.36 (s, 1H).

LC-MS (Method 1): Rₜ=0.64 min; MS (ESIneg): m/z=660 [M−H—HCl]⁻.

### Example 164

### 4′-[(2S)-2-({[trans-4-(Aminomethyl)cyclohexyl]carbonyl}amino)-3-(1H-indazol-6-ylamino)-3-oxopropyl]-N-(3-hydroxycyclobutyl)-2-methylbiphenyl-4-carboxamide hydrochloride

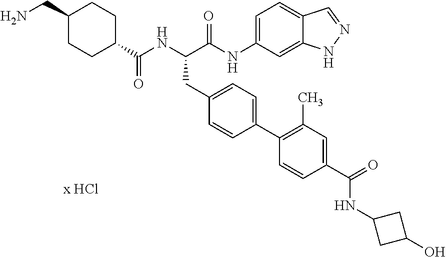 [SMILES: Cc1cc(C(=O)NC2CC(O)C2)ccc1-c1ccc(C[C@H](NC(=O)[C@H]2CC[C@H](CN)CC2)C(=O)Nc2ccc3cn[nH]c3c2)cc1]

A solution of tert-butyl [(trans-4-{[(2S)-3-{4′-[(3-hydroxycyclobutyl)carbamoyl]-2′-methylbiphenyl-4-yl}-1-(1H-indazol-6-ylamino)-1-oxopropan-2-yl]carbamoyl}cyclohexyl)methyl]-carbamate (85.2 mg, 0.12 mmol) in dioxane (3 ml) was admixed with 4M hydrogen chloride in 1,4-dioxane (0.44 ml, 1.8 mmol) and stirred at RT overnight. The solvent was removed on a rotary evaporator and the residue was dissolved in DMSO/acetonitrile (about 1 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 50.2 mg (63% of theory) of the title compound.

¹H NMR (400 MHz, DMSO-d₆): δ=ppm 0.85-1.00 (m, 2H), 1.12-1.33 (m, 2H), 1.49 (br. s., 1H), 1.58 (d, 1H), 1.69-1.82 (m, 3H), 1.93 (m, 2H), 2.11-2.35 (m, 5H), 2.53-2.58 (m, 1H), 2.62 (m, 2H), 2.98 (dd, 1H), 3.14 (dd, 1H), 3.82-3.94 (m, 2H), 4.78 (td, 1H), 7.18 (d, 1H), 7.21 (d, 1H), 7.26 (d, 2H), 7.41 (d, 2H), 7.67 (d, 1H), 7.72 (d, 1H), 7.78 (s, 1H), 7.94 (br. s., 3H), 7.98 (s, 1H), 8.14 (s, 1H), 8.28 (d, 1H), 8.52-8.61 (m, 1H), 10.39 (s, 1H).

LC-MS (Method 1): Rₜ=0.63 min; MS (ESIneg): m/z=621 [M−H—HCl]⁻.

### Example 165

### 4′-({(2S)-2-({[trans-4-(Aminomethyl)cyclohexyl]carbonyl}amino)-3-oxo-3-[(4-{5-[1,1,2,2-tetrafluoro-3-(methylamino)-3-oxopropyl]-4H-1,2,4-triazol-3-yl}phenyl)amino]propyl}-N-isopropyl-2-methylbiphenyl-4-carboxamide hydrochloride

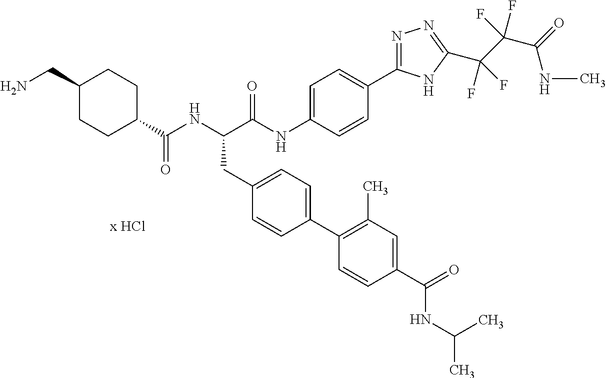 [SMILES: CNC(=O)C(F)(F)C(F)(F)c1nnc(-c2ccc(NC(=O)[C@H](Cc3ccc(-c4ccc(C(=O)NC(C)C)cc4C)cc3)NC(=O)[C@H]3CC[C@H](CN)CC3)cc2)[nH]1]

A solution of tert-butyl {[trans-4-({(2S)-3-[4′-(isopropylcarbamoyl)-2′-methylbiphenyl-4-yl]-1-oxo-1-[(4-{5-[1,1,2,2-tetrafluoro-3-(methylamino)-3-oxopropyl]-4H-1,2,4-triazol-3-yl}phenyl)-amino]propan-2-yl}carbamoyl)cyclohexyl]methyl}carbamate (81.9 mg, 0.093 mmol) in dioxane (3.5 ml) was admixed with 4M hydrogen chloride in 1,4-dioxane (0.35 ml, 1.40 mmol) and stirred at RT overnight. The precipitated solid was filtered off and washed with dioxane/acetonitrile. The solid was dried under high vacuum. This gave 75.6 mg (98% of theory) of the title compound.

¹H NMR (400 MHz, DMSO-d₆): δ=ppm 0.80-1.03 (m, 2H), 1.12-1.34 (m, 1H), 1.16 (s, 3H), 1.18 (s, 3H), 1.47 (br. s, 1H), 1.58 (d, 1H), 1.74 (d, 3H), 2.09-2.20 (m, 1H), 2.23 (s, 3H), 2.64 (m, 2H), 2.71 (d, 3H), 2.96 (dd, 1H), 3.15 (dd, 1H), 4.05-4.18 (m, 1H), 4.69-4.81 (m, 1H), 7.22 (d, 1H), 7.26 (d, 2H), 7.40 (d, 2H), 7.69 (dd, 1H), 7.73-7.86 (m, 6H), 7.98 (d, 2H), 8.19 (d, 1H), 8.25 (d, 1H), 9.05 (d, 1H), 10.49 (br. s., 1H), 15.11 (br. s, 1H).

LC-MS (Method 1): Rₜ=0.77 min; MS (ESIneg): m/z=777 [M−H—HCl]⁻.

### Example 166

### 3-[5-(4-{[(2S)-2-({[trans-4-(Aminomethyl)cyclohexyl]carbonyl}amino)-3-(2′-methyl-4′-{[(3R)-2-oxopiperidin-3-yl]carbamoyl}biphenyl-4-yl)propanoyl]amino}phenyl)-4H-1,2,4-triazol-3-yl]-2,2,3,3-tetrafluoropropanoic acid hydrochloride

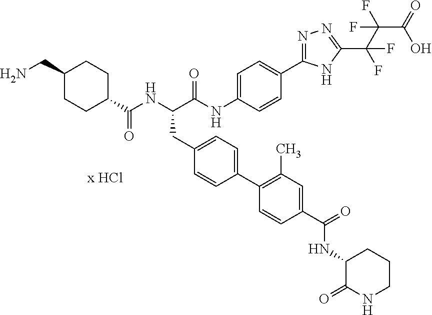 [SMILES: Cc1cc(C(=O)N[C@@H]2CCCNC2=O)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

To a solution of 112 mg (121 μmol) of 3-[5-(4-{[(2S)-2-{[(trans-4-{[(tert-butoxycarbonyl)amino]methyl}cyclohexyl)carbonyl]amino}-3-(2′-methyl-4′-{[(3R)-2-oxopiperidin-3-yl]carbamoyl}biphenyl-4-yl)propanoyl]amino}phenyl)-4H-1,2,4-triazol-3-yl]-2,2,3,3-tetrafluoropropanoic acid in 3.5 ml of dioxane were added 152 μl (0.61 mmol) of 4M hydrogen chloride in dioxane. The mixture was stirred at RT for 18 h. Acetonitrile was added and the solid obtained was filtered, washed with acetonitrile and dried under high vacuum. The residue in 3.5 ml of dioxane was admixed with 152 μl (0.61 mmol) of 4M hydrogen chloride in dioxane. The mixture was stirred at RT for 18 h. Acetonitrile was added and the solid obtained was filtered, washed with acetonitrile and dried under high vacuum. 89 mg (81% of theory) of the title compound were obtained.

¹H NMR (400 MHz, DMSO-d₆): δ=ppm 0.80-1.01 (m, 2H), 1.12-1.34 (m, 2H), 1.39-1.52 (m, 1H), 1.55-1.62 (m, 1H), 1.68-1.89 (m, 4H), 1.96-2.06 (m, 1H), 2.10-2.19 (m, 1H), 2.24 (s, 3H), 2.58-2.69 (m, 2H), 2.91-2.99 (m, 1H), 3.08-3.22 (m, 3H), 3.38 (m, 2H), 4.30-4.43 (m, 1H), 4.30-4.42 (m, 1H), 4.67-4.81 (m, 1H), 7.10-7.33 (m, 3H), 7.40 (d, 2H), 7.57-7.85 (m, 7H), 7.98 (d, 2H), 8.23 (d, 1H), 8.55 (d, 1H), 10.45 (s, 1H), 15.11 (br. s, 1H).

LC-MS (Method 12): Rₜ=1.57 min; MS (ESIneg): m/z=819.2 [M−H—HCl]⁻.

### Example 167

### 4′-[(2S)-2-({[trans-4-(Aminomethyl)cyclohexyl]carbonyl}amino)-3-(1H-indazol-6-ylamino)-3-oxopropyl]-N-cyclopropyl-2-methylbiphenyl-4-carboxamide hydrochloride

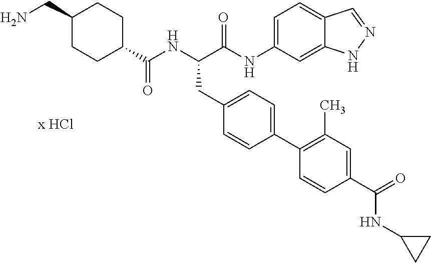 [SMILES: Cc1cc(C(=O)NC2CC2)ccc1-c1ccc(C[C@H](NC(=O)[C@H]2CC[C@H](CN)CC2)C(=O)Nc2ccc3cn[nH]c3c2)cc1]

A solution of tert-butyl [(trans-4-{[(2S)-3-[4′-(cyclopropylcarbamoyl)-2′-methylbiphenyl-4-yl]-1-(1H-indazol-6-ylamino)-1-oxopropan-2-yl]carbamoyl}cyclohexyl)methyl]carbamate (48 mg, 0.069 mmol) in dioxane (2 ml) was admixed with 4M hydrogen chloride in 1,4-dioxane (0.26 ml, 1.04 mmol) and stirred at RT for 3 days. The precipitated solid was filtered off and washed with acetonitrile. The solid was dried under high vacuum. This gave 46.9 mg (94% of theory) of the title compound.

¹H NMR (400 MHz, DMSO-d₆): δ=ppm 0.53-0.61 (m, 2H), 0.66-0.74 (m, 2H), 0.82-1.00 (m, 2H), 1.08-1.34 (m, 2H), 1.47 (br. s., 1H), 1.57 (d, 1H), 1.67-1.83 (m, 3H), 2.16 (m, 1H), 2.21 (s, 3H), 2.63 (t, 2H), 2.85 (m, 1H), 2.96 (dd, 1H), 3.13 (dd, 1H), 4.70-4.84 (m, 1H), 7.15 (d, 1H), 7.21 (d, 1H), 7.26 (d, 2H), 7.40 (d, 2H), 7.67 (m, 2H), 7.73 (s, 1H), 7.85 (br. s., 3H), 7.98 (s, 1H), 8.14 (s, 1H), 8.27 (d, 1H), 8.43 (d, 1H), 10.36 (br. s., 1H), 13.01 (br. s, 1H).

LC-MS (Method 1): Rₜ=0.71 min; MS (ESIneg): m/z=591 [M−H—HCl]⁻.

### Example 168

### 3-[5-(4-{[(2S)-2-({[trans-4-(Aminomethyl)cyclohexyl]carbonyl}amino)-3-{4′-[(trans-4-hydroxycyclohexyl)carbamoyl]-2′-methylbiphenyl-4-yl}propanoyl]amino}phenyl)-4H-1,2,4-triazol-3-yl]-2,2,3,3-tetrafluoropropanoic acid (enantiomer 1)

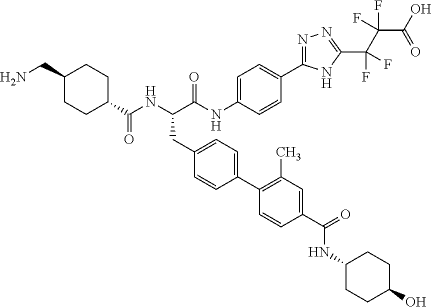 [SMILES: Cc1cc(C(=O)N[C@H]2CC[C@H](O)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]

Lithium hydroxide (734 mg, 30.64 mmol) was dissolved in 12.0 ml of water while heating and diluted with 120.0 ml of THF. Subsequently, methyl 3-[5-(4-{[(2S)-2-({[trans-4-(aminomethyl)cyclohexyl]carbonyl}amino)-3-(2′-methyl-4′-{[trans-4-(2,2,2-trifluoroacetoxy)-cyclohexyl]carbamoyl}biphenyl-4-yl)propanoyl]amino}phenyl)-4H-1,2,4-triazol-3-yl]-2,2,3,3-tetrafluoropropanoate trifluoroacetate (enantiomer 1) (3.21 g, 3.06 mmol) was added and the mixture was stirred at RT for 45 min. Subsequently, THF was distilled off, and the aqueous residue was diluted with 25 ml of water and acidified with 4M hydrochloric acid while cooling with ice until the pH was 3. The suspension obtained was diluted with 25 ml of water and stirred at RT for 30 min. Subsequently, the solid was filtered off, washed twice with 50 ml each time of water and dried at 50° C. under high vacuum. 2.41 g (95% of theory) of the title compound were obtained.

1H NMR (400 MHz, DMSO-d6): δ=ppm 14.3-15.3 (br. s., 1H), 10.36 (br. s., 1H), 8.28-8.19 (m, 1H), 8.18-8.11 (m, 1H), 7.98-7.90 (m, 2H), 7.81-7.65 (m, 7H), 7.38 (d, 2H), 7.28-7.18 (m, 3H), 4.79-4.68 (m, 1H), 4.58-4.52 (m, 1H), 3.80-3.65 (m, 1H), 3.45-3.36 (m, 2H, partly concealed by water signal), 3.18-3.08 (m, 1H), 3.01-2.89 (m, 1H), 2.67-2.60 (m, 2H), 2.22 (s, 3H), 2.20-2.11 (m, 1H), 1.90-1.66 (m, 7H), 1.64-1.54 (m, 1H), 1.50-1.12 (m, 7H), 1.00-0.83 (m, 2H)

LC-MS (Method 1): Rt=0.64 min; MS (ESIneg): m/z=821 [M−H]⁻.

### Example 169

### 4′-[(2R)-2-({[trans-4-(Aminomethyl)cyclohexyl]carbonyl}amino)-3-(1H-indazol-6-ylamino)-3-oxopropyl]-N-isopropyl-2-methylbiphenyl-4-carboxamide hydrochloride (enantiomer 1)

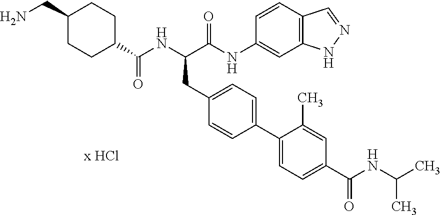

Enantiomer separation of 16 mg of the enantiomer mixture from Example 21 gave 4.3 mg of the title compound (enantiomer 1).

Chiral analytical HPLC: Rₜ=6.16 min; chiral HPLC: Rₜ=10.0-13.5 min; 98% ee.

Separation method (system: Agilent: Prep 1200, 2×Prep Pump, DLA, MWD, Prep FC): column: Chiralpak IB 5 μm 250 mm×20 mm; eluent A: water+0.1% formic acid (99%), eluent B: acetonitrile, gradient: 0-10 min 1-99% B, 10-12 min 100% B; temperature: RT; flow rate: 15 ml/min; UV detection: 254 nm.

Analysis (system: Agilent 1290 UPLC-MS: Binary Solvent Manager, Sample Manager, Column Manager, PDA, MSD 6550): column: Chiralpak IB 3μ 100 mm×4.6 mm; eluent A: water+0.1% formic acid, eluent B: acetonitrile, gradient: 0-7 min 20-40% B, 7-10 min 40% B; temperature: 25° C.; flow rate: 1 ml/min; UV detection: 254 nm.

Specific optical rotation (P2000 polarimeter): [α]=−40.8+/−1.160 (1.53 mg/ml DMSO-D₆, 20° C., 589 nm).

### Example 170

### 4′-[(2S)-2-({[trans-4-(Aminomethyl)cyclohexyl]carbonyl}amino)-3-(1H-indazol-6-ylamino)-3-oxopropyl]-N-isopropyl-2-methylbiphenyl-4-carboxamide hydrochloride (enantiomer 2)

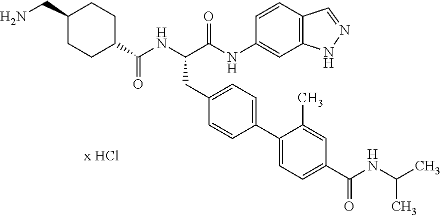

Enantiomer separation of 16 mg of the enantiomer mixture from Example 21 gave 4.6 mg of the title compound (enantiomer 2).

Chiral analytical HPLC: Rₜ=7.48 min; chiral HPLC: Rₜ=16.5-19 min; 98% ee.

Separation method (system: Agilent: Prep 1200, 2×Prep Pump, DLA, MWD, Prep FC): column: Chiralpak IB 5 μm 250 mm×20 mm; eluent A: water+0.1% formic acid (99%), eluent B: acetonitrile, gradient: 0-10 min 1-99% B, 10-12 min 100% B; temperature: RT; flow rate: 15 ml/min; UV detection: 254 nm.

Analysis (system: Agilent 1290 UPLC-MS: Binary Solvent Manager, Sample Manager, Column Manager, PDA, MSD 6550): column: Chiralpak IB 3μ 100 mm×4.6 mm; eluent A: water+0.1% formic acid, eluent B: acetonitrile, gradient: 0-7 min 20-40% B, 7-10 min 40% B; temperature: 25° C.; flow rate: 1 ml/min; UV detection: 254 nm.

Specific optical rotation (P2000 polarimeter): [α]=+38.30+/−1.060 (1.58 mg/ml DMSO-D₆, 20° C., 589 nm).

### B) Assessment of Physiological Efficacy

The suitability of the inventive compounds for treatment of thromboembolic or hyperfibrinolytic disorders can be demonstrated in the following assay systems:

### a) Test Descriptions (In Vitro)

### a. 1) Measurement of FXIa Inhibition

The factor XIa inhibition of the inventive substances is determined using a biochemical test system which utilizes the reaction of a peptidic factor XIa substrate to determine the enzymatic activity of human factor XIa. Here, factor XIa cleaves from the peptic factor XIa substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). Subsequently, the following are added successively: 20 μl of assay buffer (50 mmol/l Tris buffer pH 7.4; 100 mmol/l sodium chloride; 5 mmol/l calcium chloride; 0.1% bovine serum albumin) and 20 μl of factor XIa from Kordia (0.45 nM in assay buffer). After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nM). The measured emissions of the test mixtures with test substance are compared to those of control mixtures without test substance (only dimethyl sulphoxide instead of test substance in dimethyl sulphoxide), and the IC₅₀ values are calculated from the concentration/activity relationships. Activity data from this test are listed in Table A below:

TABLE A

| Example No. | IC₅₀ [nM] | Example No. | IC₅₀ [nM] |
|---|---|---|---|
| 1 | 2 | 2 | 4.9 |
| 3 | 11 | 4 | 11.9 |
| 5 | 16 | 6 | 17 |
| 7 | 1.7 | 8 | 2.5 |
| 9 | 1.7 | 10 | 4.4 |
| 11 | 2.5 | 12 | 2.7 |
| 13 | 4.2 | 14 | 1.4 |
| 15 | 2.9 | 16 | 3.9 |
| 17 | 6.1 | 18 | 3.7 |
| 19 | 2.9 | 20 | 1.8 |
| 21 | 1.8 | 22 | 15 |
| 23 | 27.5 | 24 | 18 |
| 25 | 14 | 26 | 2.4 |
| 27 | 2.8 | 28 | 14 |
| 29 | 3 | 30 | 3.2 |
| 31 | 30 | 32 | 3.4 |
| 33 | 0.78 | 34 | 1.4 |
| 35 | 2.3 | 36 | 1.7 |
| 37 | 8.5 | 38 | 14 |
| 39 | 9.8 | 40 | 3.6 |
| 41 | 6.0 | 42 | 3.6 |
| 43 | 3.3 | 44 | 5.7 |
| 45 | 6.2 | 46 | 3.6 |
| 47 | 16 | 48 | 2.7 |
| 49 | 2.1 | 50 | 8.3 |
| 51 | 11 | 52 | 14 |
| 53 | 16 | 54 | 16 |
| 55 | 41 | 56 | 48 |
| 57 | 8.4 | 58 | 41 |
| 59 | 34 | 60 | 45 |
| 61 | 3.2 | 62 | 2.9 |
| 63 | 2.1 | 64 | 1.6 |
| 65 | 26 | 66 | 29 |
| 67 | 4.6 | 68 | 8.3 |
| 69 | 2.4 | 70 | 1.6 |
| 71 | 7.4 | 72 | — |
| 73 | 3.4 | 74 | 1.3 |
| 75 | 2.5 | 76 | 25 |
| 77 | 8.4 | 78 | 12 |
| 79 | 12 | 80 | 17 |
| 81 | 22 | 82 | 21 |
| 83 | 2.5 | 84 | 18 |
| 85 | 16 | 86 | 13 |
| 87 | 2.8 | 88 | 2.1 |
| 89 | 4.3 | 90 | 1.6 |
| 91 | 7.2 | 92 | 1.8 |
| 93 | 2.4 | 94 | 9.2 |
| 95 | 2.4 | 96 | 1.3 |
| 97 | 3.2 | 98 | 1.0 |
| 99 | 4.7 | 100 | 2.2 |
| 101 | 1.1 | 102 | 7.9 |
| 103 | 1.1 | 104 | 0.4 |
| 105 | 65 | 106 | 1.1 |
| 107 | 1.4 | 108 | 1.9 |
| 109 | 3.0 | 110 | 1.8 |
| 111 | 1.0 | 112 | 2.0 |
| 113 | 4.9 | 114 | 2.6 |
| 115 | 2.3 | 116 | 2.0 |
| 117 | 8.9 | 118 | 2.9 |
| 119 | 2.1 | 120 | 6.7 |
| 121 | 1.0 | 122 | 5.2 |
| 123 | 2.5 | 124 | 1.8 |
| 125 | 3.0 | 126 | 1.6 |
| 127 | 1.5 | 128 | 1.7 |
| 129 | 1.8 | 132 | 1.6 |
| 133 | 6.7 | 134 | 8.9 |
| 135 | 2.2 | 136 | 1.2 |
| 137 | 11 | 138 | 0.84 |
| 139 | 4.1 | 140 | 13 |
| 141 | 3.9 | 142 | 15 |
| 143 | 2.3 | 144 | 1.8 |
| 145 | 3.2 | 146 | 1.8 |
| 147 | 1.7 | 148 | 1.0 |
| 149 | 1.8 | 150 | 2.5 |
| 151 | 0.97 | 152 | 5.3 |
| 153 | 1.8 | 154 | 2.8 |
| 155 | 1.0 | 156 | 7.1 |
| 157 | 3.6 | 158 | 3.8 |
| 159 | 3.5 | 160 | 5.2 |
| 161 | 6.1 | 162 | 1.8 |
| 163 | 10 | 164 | 7.6 |
| 165 | 1.4 | 166 | 0.8 |
| 167 | 6.0 | 168 | 0.5 |
| 169 | 2800 | 170 | 5.6 |

### a.2) Determination of the Selectivity

To demonstrate the selectivity of the substances with respect to FXIa inhibition, the test substances are examined for their inhibition of other human serin proteases, such as factor Xa, trypsin and plasmin. To determine the enzymatic activity of factor Xa (1.3 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of sodium chloride, 0.1% BSA [bovine serum albumin], 5 mmol/l of calcium chloride, pH 7.4) and incubated for 15 min with test substance in various concentrations in dimethyl sulphoxide and also with dimethyl sulphoxide without test substance. The enzymatic reaction is then started by addition of the appropriate substrates (5 μmol/l of Boc-Ile-Glu-Gly-Arg-AMC from Bachem for factor Xa and trypsin, 50 μmol/l of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulphoxide instead of test substance in dimethyl sulphoxide) and the IC₅₀ values are calculated from the concentration/activity relationships.

### a.3) Thrombin Generation Assay (Thrombogram)

The effect of the test substances on the thrombogram (thrombin generation assay according to Hemker) is determined in vitro in human plasma (Octaplas® from Octapharma).

In the thrombin generation assay according to Hemker, the activity of thrombin in coagulating plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 pM or 0.1 pM recombinant tissue factor, 24 μM phospholipids in HEPES) are used. In addition, a thrombin calibrator from Thrombinoscope is used whose amidolytic activity is required for calculating the thrombin activity in a sample containing an unknown amount of thrombin. The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM HEPES, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nM filter pair and a dispenser.

Using the Thrombinoscope software, the thrombogram is calculated and represented graphically. The following parameters are calculated: lag time, time to peak, peak, ETP (endogenous thrombin potential) and start tail.

### a.4) Determination of Anticoagulatory Activity

The anticoagulatory activity of the test substances is determined in vitro in human and animal plasma (for example mouse, rat, rabbit, pig and dog plasma). To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The urothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

The activated partial thromboolastin time (aPTT) is determined in the presence of varying concentrations of test substance or the corresponding solvent using a commercial test kit (C. K. Prest from Diagnostica Stago). The test compounds are incubated with the plasma and the PTT reagent (cephalin, kaolin) at 37° C. for 3 minutes. Coagulation is then started by addition of a 25 mM aqueous calcium chloride solution, and the time when coagulation occurs is determined. The concentration of test substance which brings about a 1.5-fold extension of the aPTT is determined. Activity data from this test are listed in Table B below:

TABLE B

| Example No. | aPTT [μmol/l] | Example No. | aPTT [μmol/l] |
|---|---|---|---|
| 1 | 0.1 | 2 | 0.14 |
| 3 | 0.2 | 4 | 0.21 |
| 5 | 0.21 | 6 | 0.21 |
| 8 | 0.09 | 9 | 0.09 |
| 10 | 0.13 | 11 | 0.08 |
| 12 | 0.1 | 13 | 0.44 |
| 14 | 0.49 | 15 | 0.09 |
| 16 | 0.23 | 17 | 0.35 |
| 18 | 0.16 | 22 | 0.06 |
| 23 | 0.28 | 24 | 0.31 |
| 25 | 0.14 | 26 | 0.19 |
| 27 | 0.24 | 28 | 0.33 |
| 29 | 0.14 | 30 | 0.12 |
| 31 | 0.22 | 32 | 0.09 |
| 33 | 0.11 | 34 | 0.11 |
| 35 | 0.11 | 36 | 0.07 |
| 37 | 0.12 | 38 | 0.42 |
| 39 | 0.1 | 40 | 0.11 |
| 41 | 0.18 | 42 | 0.1 |
| 43 | 0.06 | 44 | 0.11 |
| 45 | 0.14 | 46 | 0.22 |
| 47 | 0.51 | 48 | 0.1 |
| 49 | 0.08 | 50 | 0.16 |
| 51 | 0.44 | 52 | 0.2 |
| 53 | 0.16 | 54 | 0.24 |
| 55 | 0.37 | 56 | 0.44 |
| 57 | 0.44 | 58 | 0.51 |
| 59 | 0.51 | 60 | 0.63 |
| 61 | 0.07 | 62 | 0.53 |
| 63 | 0.01 | 64 | 0.08 |
| 65 | 0.48 | 66 | 0.32 |
| 67 | 0.12 | 68 | 0.15 |
| 69 | 0.22 | 70 | 0.08 |
| 71 | 0.1 | 72 | 0.15 |
| 73 | 0.22 | 74 | 0.09 |
| 75 | 0.06 | 76 | 0.37 |
| 77 | 0.22 | 78 | 0.38 |
| 79 | 0.21 | 80 | 0.41 |
| 81 | 0.41 | 82 | 0.39 |
| 83 | 0.09 | 84 | 0.3 |
| 85 | 0.12 | 86 | 0.14 |
| 87 | 0.47 | 88 | 0.31 |
| 89 | 0.03 | 90 | 0.06 |
| 91 | 0.14 | 92 | 0.15 |
| 93 | 0.21 | 94 | 0.23 |
| 95 | 0.33 | 96 | 0.5 |
| 97 | 0.06 | 98 | 0.06 |
| 99 | 0.27 | 100 | 0.1 |
| 101 | 0.07 | 102 | — |
| 103 | 0.1 | 104 | 0.05 |
| 106 | 0.11 | 107 | 0.1 |
| 108 | 0.07 | 109 | 0.11 |
| 110 | 0.11 | 111 | 0.13 |
| 112 | 0.17 | 113 | 0.07 |
| 114 | 0.14 | 115 | 0.09 |
| 116 | 0.57 | 117 | 0.18 |
| 118 | 0.11 | 119 | 0.08 |
| 120 | 0.14 | 121 | 0.007 |
| 122 | 0.16 | 123 | 0.02 |
| 124 | 0.1 | 125 | 0.09 |
| 126 | 0.14 | 127 | 0.14 |
| 128 | 0.14 | 129 | 0.09 |
| 130 | 0.12 | 131 | 0.2 |
| 132 | 0.05 | 133 | 0.1 |
| 134 | 0.44 | 135 | 0.24 |
| 136 | 0.29 | 137 | 0.07 |
| 138 | 0.25 | 139 | 0.17 |
| 140 | 0.25 | 141 | 0.30 |
| 142 | 0.33 | 143 | 0.07 |
| 144 | 0.06 | 145 | 0.07 |
| 146 | 0.07 | 147 | 0.01 |
| 148 | 0.07 | 149 | 0.04 |
| 150 | 0.07 | 151 | 0.1 |
| 152 | 0.7 | 153 | 0.08 |
| 154 | 0.09 | 155 | 0.16 |
| 157 | 0.33 | 158 | 0.1 |
| 159 | 0.36 | 162 | 0.15 |
| 163 | 0.07 | 164 | 0.13 |
| 166 | 0.09 | 167 | 0.12 |

### a.5) Determination Offibrinolvtic Activity

Antifibrinolytic activity in vitro is assessed in human, platelet-free plasma. Tissue factor (TF) (1 pM) and tissue plasminogen activator (tPA) (40 nM) are pipetted into plasma together with 12.5 mM aqueous calcium chloride solution and substance. On occurrence of clotting, the subsequent clot lysis is determined photometrically over a period of 30 minutes.

### a.6) Measurement of Plasmin Inhibition

The plasmin inhibition of the inventive substances is determined using a biochemical test system which utilizes the reaction of a peptidic plasmin substrate to determine the enzymatic activity of human plasmin. Here, plasmin cleaves from the peptic plasmin substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions are placed into the wells of white microtitre plates from Greiner (384 wells). Subsequently, the following are added successively: 20 μl of assay buffer (50 mmol/Tris buffer pH 7.4; 100 mmol/l sodium chloride; 5 mmol/calcium chloride; 0.1% bovine serum albumin) and 20 μl of plasmin from Kordia (0.3 μg/ml in assay buffer) are then added successively. After 15 min of incubation, the enzyme reaction is started by addition of 20 μl of the plasmin substrate MeOSuc-Ala-Phe-Lys-AMC dissolved in assay buffer (150 μM in assay buffer) from Bachem, the mixture is incubated at room temperature (22° C.) for 30 min and fluorescence is then measured (excitation: 360 nm, emission: 460 nM). The measured emissions of the test batches with test substance are compared to those of control batches without test substance (only dimethyl sulphoxide instead of test substance in dimethyl sulphoxide), and the IC₅₀ values are calculated from the concentration/activity relationships. Activity data from this test are listed in Table C below:

TABLE C

| Example No. | IC₅₀ [nM] | Example No. | IC₅₀ [nM] |
|---|---|---|---|
| 7 | 1.9 | 20 | 5.7 |
| 21 | 1.1 | 96 | 1.4 |
| 102 | 0.74 | 116 | 4.4 |
| 133 | 1.6 | 134 | 0.64 |
| 135 | 0.96 | 136 | 1.5 |
| 137 | 2.2 | 138 | 2.3 |
| 139 | 13 | 140 | 1.5 |
| 141 | 1.6 | 142 | 2.1 |
| 156 | 0.58 | 157 | 1.7 |
| 158 | 0.43 | 159 | 0.32 |
| 160 | 1.3 | 161 | 6.7 |
| 162 | 1.1 | 163 | 8.8 |
| 164 | 13 | 165 | 1.9 |
| 167 | 1.9 | 169 | 270 |
| 170 | 1.3 | | |

### b) Determination of Antithrombotic Activity (In Vivo)

b.1) Arterial Thrombosis Model (Iron(I) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined.

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is furthermore maintained by intravenous administration of the same preparations (bolus: continuous infusion) via the right auricular vein.

The right carotid artery is exposed and the tissue injury is then caused by wrapping a piece of filter paper (10 mm×10 mm) on a Parafilm® strip (25 mm×12 mm) around the carotid artery without disturbing the blood flow. The filter paper contains 100 μl of a 13% strength solution of iron(II) chloride (Sigma) in water. After 5 min, the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3-mm-long incision (blade Art. Number 10-150-10, Martin, Tuttlingen, Germany) is made parallel to the longitudinal axis of the ear. Care is taken here not to damage any visible vessels. Any blood that extravasates is taken up in 15 second intervals using accurately weighed filter paper pieces, without touching the wound directly. Bleeding time is calculated as the time from making the incision to the point in time where no more blood can be detected on the filter paper. The volume of the extravasated blood is calculated after weighing of the filter paper pieces.

### b) Determination of Fibrinolytic Activity (In Vivo)

### c.1) Hyperfibrinolytic Rats

The determination of antifibrinolytic activity in vivo is conducted in hyperfibrinolytic rats. After anaesthetization and catheterization of the animals, hyperfibrinolysis is triggered by infusion of tissue plasminogen activator (tPA) (8 mg/kg/h). 10 minutes after commencement of tPA infusion, the substances are administered as an i.v. bolus. After a further 15 minutes, tPA infusion is ended and a transsection of the tail is conducted. Subaqual bleeding (in physiological saline at 37° C.) is observed over 30 minutes and the bleed time is determined.

### C) Working Examples of Pharmaceutical Formulations

The inventive substances can be converted to pharmaceutical preparations, for example, as follows:

### Tablet:

### Composition:

100 mg of the compound of Example 1, 50 mg of lactose (monohydrate), 50 mg of maize starch, 10 mg of polyvinylpyrrolidone (PVP) and 2 mg of magnesium stearate.

Tablet weight 212 mg, diameter 8 mm, radius of curvature 12 mm.

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tablet press (see above for format of the tablet).

### Oral Suspension:

### Composition:

1000 mg of the compound of Example 1, 1000 mg of ethanol (96%), 400 mg of Rhodigel and 99 g of water.

A single dose of 100 mg of the inventive compound corresponds to 10 ml of oral suspension.

### Production:

The Rhodigel is suspended in ethanol, and the compound of Example 1 is added to the suspension. The water is added while stirring. The mixture is stirred for about 6 h until swelling of the Rhodigel is complete.

### Solution for Oral Administration:

### Composition:

500 mg of the inventive compound, 2.5 g of polysorbate and 97 g of polyethylene glycol 400. A single dose of 100 mg of the inventive compound corresponds to 20 g of oral solution.

### Production:

The inventive compound is suspended in the mixture of polyethylene glycol and polysorbate while stirring. The stirring operation is continued until dissolution of the inventive compound is complete.

i.v. Solution:

The inventive compound is dissolved in a concentration below the saturation solubility in a physiologically acceptable solvent (e.g. isotonic saline, glucose solution 5% and/or polyethylene glycol 400/water 30% m/m). The solution is subjected to sterile filtration and dispensed into sterile and pyrogen-free injection vessels.

1. A compound of the formula 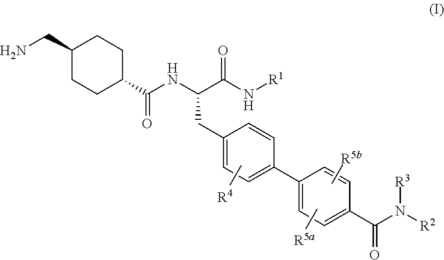
in which
R¹ is a group of the formula 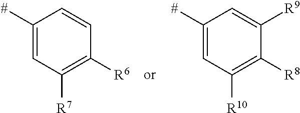
where # is the attachment site to the nitrogen atom,
R⁶ is 5-membered heteroaryl,
where heteroaryl may be substituted by a substituent selected from the group consisting of oxo, chlorine, cyano, hydroxyl and C₁-C₃-alkyl,
in which alkyl may be substituted by 1 to 3 substituents selected independently from the group consisting of hydroxyl, amino, hydroxycarbonyl and methoxy, or
in which alkyl may be substituted by 1 to 7 fluorine substituents, or
in which alkyl is substituted by a substituent selected from the group consisting of hydroxyl, amino, hydroxycarbonyl, methoxy, C₁-C₄-alkoxycarbonyl, aminocarbonyl and C₁-C₃-alkylaminocarbonyl, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
where the heterocycle may be substituted by 1 to 2 substituents selected independently from the group consisting of oxo, chlorine, cyano, hydroxyl, hydroxycarbonyl, C₁-C₃-alkyl, pyrazolyl and pyridyl,
in which alkyl may be substituted by 1 to 3 substituents selected independently from the group consisting of hydroxyl, amino, hydroxycarbonyl and methoxy, or
in which alkyl may be substituted by 1 to 7 fluorine substituents, or
in which alkyl is substituted by a substituent from the group consisting of hydroxyl, amino, hydroxycarbonyl, methoxy, C₁-C₄-alkoxycarbonyl, aminocarbonyl and C₁-C₃-alkylaminocarbonyl, and in which alkyl is additionally substituted by 1 to 6 fluorine substituents,
R¹⁰ is hydrogen, fluorine, chlorine or hydroxycarbonyl,
R² is hydrogen, C₁-C₆-alkyl, C₃-C₆-cycloalkyl, or 4- to 8-membered heterocyclyl bonded via a carbon atom,
where alkyl may be substituted by 1 to 2 substituents selected independently from the group consisting of fluorine, hydroxyl, amino, hydroxycarbonyl, C₁-C₃-alkylamino, difluoromethyl, trifluoromethyl, —(OCH₂CH₂)ₙ—OCH₃, —(OCH₂CH₂)ₘ—OH, trimethylaminium, pyrrolidinyl, C₃-C₆-cycloalkyl, 4- to 8-membered heterocyclyl bonded via a carbon atom, and 4- to 6-membered heterocyclylcarbonyl,
in which n is a number from 1 to 6,
in which m is a number from 1 to 6,
in which heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl, and
in which heterocyclylcarbonyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl, and
where cycloalkyl may be substituted by 1 to 3 independently selected from the group consisting of oxo, fluorine, hydroxyl, amino, C₁-C₄-alkyl and C₁-C₃-alkylamino,
in which alkyl and alkylamino may be substituted by 1 to 5 fluorine substituents or one phenyl substituent, and
where heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
where alkyl and alkylamino may be substituted by 1 to 5 substituents selected independently from the group consisting of hydroxyl and fluorine, and
where heterocyclyl may additionally be substituted by 1 to 4 substituents independently selected from the group consisting of fluorine and methyl,
R³ is hydrogen or C₁-C₃-alkyl, or
R² and R³ together with the nitrogen atom to which they are bonded form a 4- to 8-membered heterocycle,
where the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R⁴ is hydrogen, fluorine, chlorine, methyl or methoxy,
R^{5a} is hydrogen, fluorine, chlorine, C₁-C₄-alkyl, methoxy, ethoxy or trifluoromethyl,
R^{5b} is hydrogen, fluorine, methyl or methoxy,
or one of the salts thereof, solvates thereof or solvates of the salts thereof.

2. The compound of claim 1, characterized in that
R¹ is a group of the formula 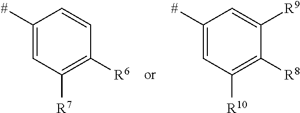
where # is the attachment site to the nitrogen atom,
R⁶ is 5-membered heteroaryl,
where heteroaryl may be substituted by a substituent selected from the group consisting of C₁-C₃-alkyl,
in which alkyl may be substituted by a hydroxycarbonyl substituent, or
in which alkyl is substituted by a hydroxycarbonyl substituent and in which alkyl is additionally substituted by 1 to 6 fluorine substituents,
R⁷ is hydrogen,
R⁸ and R⁹ together with the carbon atoms to which they are bonded form a 5-membered heterocycle,
where the heterocycle may be substituted by 1 to 2 substituents selected independently from the group consisting of oxo, hydroxyl, methyl, ethyl and n-propyl,
in which methyl, ethyl and n-propyl may be substituted by a hydroxycarbonyl substituent, or
in which ethyl and n-propyl may be substituted by 4 to 7 fluorine substituents, or
in which ethyl and n-propyl are substituted by a hydroxycarbonyl substituent and in which ethyl and n-propyl are additionally substituted by 4 to 6 fluorine substituents,
R¹⁰ is hydrogen, fluorine or chlorine,
R² is hydrogen, C₁-C₆-alkyl, C₃-C₆-cycloalkyl, or 4- to 8-membered heterocyclyl bonded via a carbon atom,
where alkyl may be substituted by 1 to 2 substituents selected independently from the group consisting of amino, C₁-C₃-alkylamino and trifluoromethyl, and
where cycloalkyl may be substituted by 1 to 2 substituents selected independently from the group consisting of hydroxyl, amino, methyl and C₁-C₃-alkylamino, and
where heterocyclyl may be substituted by 1 to 2 substituents selected independently from the group consisting of oxo, fluorine, hydroxycarbonyl, C₁-C₄-alkyl, C₁-C₃-alkylamino, 2,2,2-trifluoroeth-1-yl and C₁-C₄-alkoxycarbonyl,
in which alkyl may be substituted by a hydroxyl substituent, and
where heterocyclyl may additionally be substituted by 1 to 2 substituents independently selected from the group consisting of fluorine and methyl,
R³ is hydrogen, or
R² and R³ together with the nitrogen atom to which they are bonded form a 4- to 6-membered heterocycle,
R⁴ is hydrogen or fluorine,
R^{5a} is hydrogen, fluorine, chlorine, methyl, methoxy or trifluoromethyl,
R^{5b} is hydrogen, fluorine, methyl or methoxy,
or one of the salts thereof, solvates thereof or solvates of the salts thereof.

3. The compound of claim 1, characterized in that
R¹ is a group of the formula 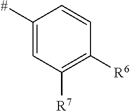
where # is the attachment site to the nitrogen atom,
R⁶ is triazolyl,
where triazolyl may be substituted by a substituent selected from the group consisting of ethyl and n-propyl,
in which ethyl and n-propyl are substituted by a hydroxycarbonyl substituent and in which ethyl and n-propyl are additionally substituted by 4 to 6 fluorine substituents,
R⁷ is hydrogen,
R² is ethyl, isopropyl, cyclopropyl, cyclobutyl, cyclohexyl, or heterocyclyl bonded via a carbon atom, selected from the group of pyrrolidinyl and piperidinyl,
where ethyl is substituted by a trifluoromethyl substituent, and
where cyclohexyl is substituted by a substituent selected from the group consisting of hydroxyl, amino and C₁-C₃-alkylamino, and
where pyrrolidinyl and piperidinyl may be substituted by 1 to 2 substituents independently selected from the group consisting of oxo, fluorine, C₁-C₄-alkyl and 2,2,2-trifluoroeth-1-yl,
R³ is hydrogen, or
R² and R³ together with the nitrogen atom to which they are bonded form a pyrrolidinyl or piperazinyl,
R⁴ is hydrogen or fluorine,
R^{5a} is hydrogen, fluorine, chlorine, methyl, methoxy or trifluoromethyl,
R^{5b} is hydrogen,
or one of the salts thereof, solvates thereof or solvates of the salts thereof.

4. The compound of claim 1, characterized in that
R¹ is a group of the formula 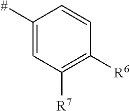
where # is the attachment site to the nitrogen atom,
R⁶ is triazolyl,
where triazolyl is substituted by a substituent selected from the group consisting of ethyl and n-propyl,
in which ethyl and n-propyl are substituted by a hydroxycarbonyl substituent and in which ethyl and n-propyl are additionally substituted by 4 to 6 fluorine substituents,
R⁷ is hydrogen,
R² is cyclohexyl, or heterocyclyl bonded via a carbon atom, selected from the group of pyrrolidinyl and piperidinyl,
where cyclohexyl is substituted by a substituent selected from the group consisting of hydroxyl, amino and C₁-C₃-alkylamino, and
where pyrrolidinyl and piperidinyl may be substituted by 1 to 2 substituents independently selected from the group consisting of oxo and methyl,
R³ is hydrogen,
R⁴ is hydrogen or fluorine,
R^{5a} is fluorine or methyl,
R^{5b} is hydrogen,
or one of the salts thereof, solvates thereof or solvates of the salts thereof.

5. The compound of claim 1, characterized in that
R¹ is a group of the formula 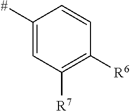
where # is the attachment site to the nitrogen atom,
R⁶ is triazolyl,
where triazolyl is substituted by a substituent selected from the group consisting of ethyl and n-propyl,
in which ethyl and n-propyl are substituted by a hydroxycarbonyl substituent and in which ethyl and n-propyl are additionally substituted by 4 to 6 fluorine substituents,
R⁷ is hydrogen,
R² is cyclohexyl,
where cyclohexyl is substituted by a substituent selected from the group consisting of hydroxyl, amino and C₁-C₃-alkylamino,
R³ is hydrogen,
R⁴ is hydrogen or fluorine,
R^{5a} is fluorine or methyl,
R^{5b} is hydrogen,
or one of the salts thereof, solvates thereof or solvates of the salts thereof.

6. The compound of claim 1, characterized in that
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl or 1H-indazol-6-yl,
where the 5-membered heterocycle in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by 1 to 2 substituents selected independently from the group consisting of oxo, methyl, ethyl and n-propyl,
in which ethyl and n-propyl may be substituted by 4 to 7 fluorine substituents, or
in which ethyl and n-propyl are substituted by a hydroxycarbonyl substituent and in which ethyl and n-propyl are additionally substituted by 4 to 6 fluorine substituents, and
where the benzyl ring in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by a substituent selected from the group consisting of fluorine and chlorine,
R² is ethyl, isopropyl, cyclopropyl, cyclobutyl, cyclohexyl, or heterocyclyl bonded via a carbon atom, selected from the group of pyrrolidinyl and piperidinyl,
where ethyl is substituted by a trifluoromethyl substituent, and
where cyclohexyl is substituted by a substituent selected from the group consisting of hydroxyl, amino and C₁-C₃-alkylamino, and
where pyrrolidinyl and piperidinyl may be substituted by 1 to 2 substituents independently selected from the group consisting of oxo and methyl,
R³ is hydrogen,
R⁴ is hydrogen or fluorine,
R^{5a} is fluorine, chlorine or methyl,
R^{5b} is hydrogen,
or one of the salts thereof, solvates thereof or solvates of the salts thereof.

7. The compound 3-[5-(4-{[(2S)-2-({[trans-4-(Aminomethyl)cyclohexyl]carbonyl}-amino)-3-{4′-[(trans-4-hydroxycyclohexyl)carbamoyl]-2′-methylbiphenyl-4-yl}propanoyl]-amino}phenyl)-4H-1,2,4-triazol-3-yl]-2,2,3,3-tetrafluoropropanoic acid (enantiomer 1) according to claim 1, having the following formula 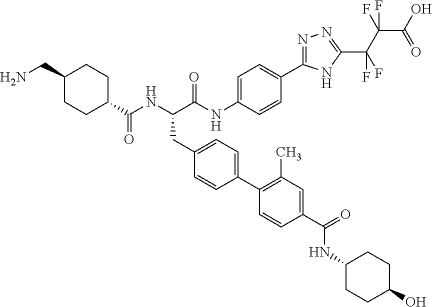 [SMILES: Cc1cc(C(=O)N[C@H]2CC[C@H](O)CC2)ccc1-c1ccc(C[C@H](NC(=O)[C@H]2CC[C@H](CN)CC2)C(=O)Nc2ccc(-c3nnc(C(F)(F)C(F)(F)C(=O)O)[nH]3)cc2)cc1]
or one of the salts thereof, solvates thereof or solvates of the salts thereof.

8. A method of making the compound of claim 1 of the formula (I) or one of the salts thereof, solvates thereof or solvates of the salts thereof, characterized in that a compound of the formula 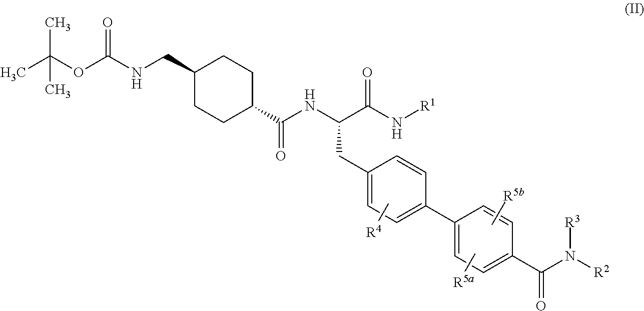
in which R¹, R², R³, R⁴, R^{5a} and R^{5b} are each as defined in claim 1, is reacted with an acid.

9. A method for treatment and/or prophylaxis of diseases using the compound of claim 1.

10. A method of making a medicament for treatment and/or prophylaxis of diseases using the compound of claim 1.

11. A method of making a medicament for the treatment and/or prophylaxis of thrombotic or thromboembolic disorders using the compound of claim 1.

12. A medicament comprising the compound of claim 1 in combination with an inert, nontoxic, pharmaceutically suitable excipient.

13. A method for treatment and/or prophylaxis of thrombotic or thromboembolic disorders using the medicament of claim 12.

14. A method for treating thrombotic or thromboembolic disorders in man and animals by administration of a therapeutically effective amount of the compound of claim 1.

15. A compound 2,2,3,3-Tetrafluoro-3-[5-(4-nitrophenyl)-1H-1,2,4-triazol-3-yl]propanoic acid having the following formula 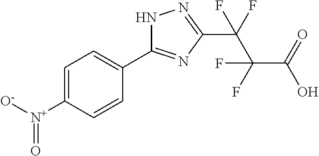 [SMILES: O=C(O)C(F)(F)C(F)(F)c1n[nH]c(-c2ccc([N+](=O)[O-])cc2)n1]
or
3-[5-(4-aminophenyl)-1H-1,2,4-triazol-3-yl]-2,2,3,3-tetrafluoropropanoic acid hydrochloride having the following formula 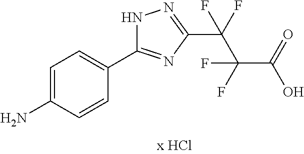 [SMILES: Nc1ccc(-c2nc(C(F)(F)C(F)(F)C(=O)O)n[nH]2)cc1]
or
methyl 2,2,3,3-tetrafluoro-3-[5-(4-nitrophenyl)-1H-1,2,4-triazol-3-yl]propanoate having the following formula 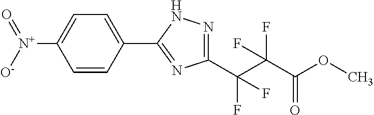 [SMILES: COC(=O)C(F)(F)C(F)(F)c1n[nH]c(-c2ccc([N+](=O)[O-])cc2)n1]
or
methyl 3-[5-(4-aminophenyl)-1H-1,2,4-triazol-3-yl]-2,2,3,3-tetrafluoropropanoate having the following formula 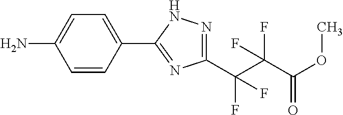 [SMILES: COC(=O)C(F)(F)C(F)(F)c1n[nH]c(-c2ccc(N)cc2)n1]
or
3-[5-(4-aminophenyl)-1H-1,2,4-triazol-3-yl]-2,2,3,3-tetrafluoro-N,N-dimethylpropanamide having the following formula 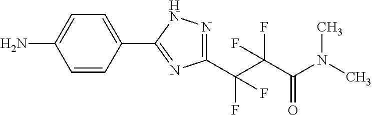 [SMILES: CN(C)C(=O)C(F)(F)C(F)(F)c1n[nH]c(-c2ccc(N)cc2)n1]
or
3-[5-(4-aminophenyl)-1H-1,2,4-triazol-3-yl]-2,2,3,3-tetrafluoropropanamide having the following formula 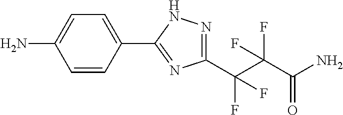 [SMILES: NC(=O)C(F)(F)C(F)(F)c1n[nH]c(-c2ccc(N)cc2)n1]
or
3-[5-(4-aminophenyl)-1H-1,2,4-triazol-3-yl]-2,2,3,3-tetrafluoro-N-methylpropanamide having the following formula 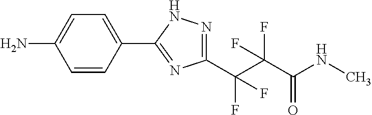 [SMILES: CNC(=O)C(F)(F)C(F)(F)c1n[nH]c(-c2ccc(N)cc2)n1]
or
3-[5-(4-aminophenyl)-1H-1,2,4-triazol-3-yl]-2,2,3,3-tetrafluoropropanoic acid having the following formula 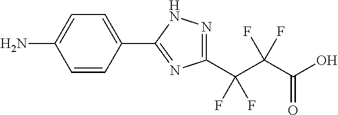 [SMILES: Nc1ccc(-c2nc(C(F)(F)C(F)(F)C(=O)O)n[nH]2)cc1]
or
2,2,3,3,4,4-hexafluoro-4-[5-(4-nitrophenyl)-4H-1,2,4-triazol-3-yl]butanoic acid having the following formula 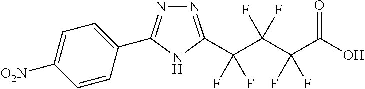 [SMILES: O=C(O)C(F)(F)C(F)(F)C(F)(F)c1nnc(-c2ccc([N+](=O)[O-])cc2)[nH]1]
or
4-[5-(4-aminophenyl)-4H-1,2,4-triazol-3-yl]-2,2,3,3,4,4-hexafluorobutanoic acid hydrochloride having the following formula 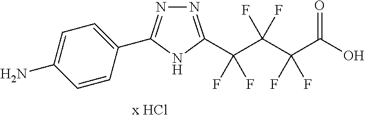 [SMILES: Nc1ccc(-c2nnc(C(F)(F)C(F)(F)C(F)(F)C(=O)O)[nH]2)cc1]
or one of the salts, the solvates or the solvates of the salts of these compounds.

16. A method for treating thrombotic or thromboembolic disorders in man and animals by administration of a therapeutically effective amount of the medicament of claim 12.

17. A method for treating thrombotic or thromboembolic disorders in man and animals by administration of a therapeutically effective amount of the medicament of claim 10.
